(19) United States
(12) Patent Application Publication
(10) Pub. No.: US 2024/0336905 A1
(43) Pub. Date: Oct. 10, 2024

(54) CLASS II, TYPE V CRISPR SYSTEMS

(71) Applicant: Metagenomi, Inc., Emeryville, CA (US)

(72) Inventors: Brian C. THOMAS, Berkeley, CA (US); Christopher BROWN, Albany, CA (US); Cindy CASTELLE, San Francisco, CA (US); Lisa ALEXANDER, Albany, CA (US); Liliana GONZALEZ-OSORIO, Hercules, CA (US); Paula MATHEUS CARNEVALI, Pinole, CA (US); Dom CASTANZO, Oakland, CA (US)

(21) Appl. No.: 18/597,260

(22) Filed: Mar. 6, 2024

Related U.S. Application Data
(63) Continuation of application No. PCT/US2022/075992, filed on Sep. 6, 2022.
(60) Provisional application No. 63/241,928, filed on Sep. 8, 2021.

(51) Int. Cl. C12N 9/22; C12N 15/11; C12N 15/90

(52) U.S. Cl. CPC C12N 9/22; C12N 15/11; C12N 15/907; C12N 2310/20; C12N 2800/80

(57) ABSTRACT

Described herein are methods, compositions, and systems derived from uncultivated microorganisms useful for gene editing.

### RELATED APPLICATIONS

This application is related to PCT Patent Application No. PCT/US2021/021259 and to PCT Patent Application No. PCT/US2022/031849, each of which is incorporated herein by this reference in its entirety.

### CROSS-REFERENCE

This application is a continuation of International Application No. PCT/US2022/075992, filed on Sep. 6, 2022, which claims the benefit of U.S. Provisional Application No. 63/241,928, entitled “CLASS II, TYPE V CRISPR SYSTEMS”, filed on Sep. 8, 2021, each of which is incorporated herein by reference in its entirety.

### BACKGROUND

Cas enzymes along with their associated Clustered Regularly Interspaced Short Palindromic Repeats (CRISPR) guide ribonucleic acids (RNAs) appear to be a pervasive (˜45% of bacteria, ˜84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SEQUENCE LISTING

The instant application contains a Sequence Listing which has been submitted electronically in XML format and is hereby incorporated by reference in its entirety. Said XML copy, created on Mar. 1, 2024, is named 55921-732301_SL.xml and is 1,115,136 bytes in size.

### SUMMARY

In some aspects, the present disclosure provides for an engineered nuclease system comprising: an endonuclease having at least 75% sequence identity to any one of SEQ ID NOs: 1-325, 420-431, 476-624, or 629 or a variant thereof; and an engineered guide RNA, wherein said engineered guide RNA is configured to form a complex with said endonuclease and said engineered guide RNA comprises a spacer sequence configured to hybridize to a target nucleic acid sequence.

In some embodiments, said guide RNA comprises a sequence with at least 80% sequence identity to the non-degenerate nucleotides of any one of SEQ ID NOs: 410-419, 432, 434, 436, 438, 440, 442, 444, 446, 448, 450, 452, 454, 456, 458, 460, 462, 464, 466, 468, 470, 472, and 474. In some embodiments, said endonuclease has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 30-33, 39, 48, 56, 57, 61, 83, 92, 100, 110, 124, 136, 145, 148, 424, 425, 429, 476, or 629. In some embodiments, said guide RNA comprises a sequence with at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to the non-degenerate nucleotides of any one of SEQ ID NOs: 414-419, 432, 434, 436, 438, 440, 442, 444, 446, 448, 450, 452, 454, 456, 458, 460, 462, 464, 466, 468, 470, 472, and 474.

In some aspects, the present disclosure provides for an engineered nuclease system comprising: an engineered guide RNA comprising a sequence with at least 80% sequence identity to the non-degenerate nucleotides of any one of SEQ ID NOs: 410-419, 432, 434, 436, 438, 440, 442, 444, 446, 448, 450, 452, 454, 456, 458, 460, 462, 464, 466, 468, 470, 472, and 474, and a class 2, type V Cas endonuclease configured to bind to said engineered guide RNA. In some embodiments, the engineered nuclease system further comprises a DNA repair template comprising a double-stranded DNA segment flanked by one or two single-stranded DNA segments. In some embodiments, said single-stranded DNA segments are conjugated to the 5′ ends of said double-stranded DNA segment. In some embodiments, said single stranded DNA segments are conjugated to the 3′ ends of said double-stranded DNA segment. In some embodiments, said single-stranded DNA segments have a length from 4 to 10 nucleotide bases.

In some embodiments, said single-stranded DNA segments have a nucleotide sequence complementary to a sequence within said spacer sequence. In some embodiments, said double-stranded DNA sequence comprises a barcode, an open reading frame, an enhancer, a promoter, a protein-coding sequence, a miRNA coding sequence, an RNA coding sequence, or a transgene.

In some embodiments, said double-stranded DNA sequence is flanked by a nuclease cut site. In some embodiments, said nuclease cut site comprises a spacer and a PAM sequence. In some embodiments, said PAM comprises a sequence of any one of SEQ ID NOs: 433, 435, 437, 439, 441, 443, 445, 447, 449, 451, 453, 455, 457, 459, 461, 463, 465, 467, 469, 471, 473, and 475. In some embodiments, said system further comprises a source of Mg²⁺. In some embodiments, said guide RNA comprises a hairpin comprising at least 8, at least 10, or at least 12 base-paired ribonucleotides. In some embodiments, said hairpin comprises 10 base-paired ribonucleotides. In some embodiments, said endonuclease comprises a sequence at least 75%, 80%, or 90% identical to any one of SEQ ID NOs: 1, 6, 15, 30, 151, 292, or 319, or a variant thereof; and said guide RNA structure comprises a sequence at least 80%, or 90% identical to the non-degenerate nucleotides of any one of SEQ ID NOs: 410-419. In some embodiments, said endonuclease comprises a sequence at least about 75%, at least about 80%, at least about 85%, at least about at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 30-33, 39, 48, 56, 57, 61, 83, 92, 100, 110, 124, 136, 145, 148, 424, 425, 429, 476, or 629; and said guide RNA structure comprises a sequence at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to the non-degenerate nucleotides of any one of SEQ ID NOs: 414-419, 432, 434, 436, 438, 440, 442, 444, 446, 448, 450, 452, 454, 456, 458, 460, 462, 464, 466, 468, 470, 472, and 474. In some embodiments, said sequence identity is determined by a BLASTP, CLUSTALW, MUSCLE, MAFFT algorithm, or a CLUSTALW algorithm with the Smith-Waterman homology search algorithm parameters. In some embodiments, said sequence identity is determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, present disclosure provides for an engineered guide ribonucleic acid (RNA) polynucleotide comprising: a DNA-targeting segment comprising a nucleotide sequence that is complementary to a target sequence in a target DNA molecule; and a protein-binding segment comprising two complementary stretches of nucleotides that hybridize to form a double-stranded RNA (dsRNA) duplex, wherein said two complementary stretches of nucleotides are covalently linked to one another with intervening nucleotides, and wherein said engineered guide ribonucleic acid polynucleotide is capable of forming a complex with a type 2, class V Cas endonuclease. In some embodiments, said type 2, class V Cas endonuclease is derived from an uncultivated organism. In some embodiments, said Cas endonuclease has at least 75% sequence identity to any one of SEQ ID NOs: 1-325, 420-431, 476-624, or 629, and targeting said complex to said target sequence of said target DNA molecule. In some embodiments, said DNA-targeting segment is positioned 3′ of both of said two complementary stretches of nucleotides. In some embodiments, said protein binding segment comprises a sequence having at least 70%, at least 80%, or at least 90% identity to the non-degenerate nucleotides of SEQ ID NO: 410-419. In some embodiments, said double-stranded RNA (dsRNA) duplex comprises at least 5, at least 8, at least 10, or at least 12 ribonucleotides.

In some aspects, the present disclosure provides for a deoxyribonucleic acid polynucleotide encoding any of the engineered guide RNAs disclosed herein.

In some aspects, the present disclosure provides for a nucleic acid comprising an engineered nucleic acid sequence optimized for expression in an organism, wherein said nucleic acid encodes a class 2, type V Cas endonuclease, and wherein said endonuclease is derived from an uncultivated microorganism, wherein the organism is not said uncultivated organism. In some embodiments, said endonuclease comprises a variant having at least 70% or at least 80% sequence identity to any one of SEQ ID NOs: 1-325, 420-431, 476-624, or 629. In some embodiments, said endonuclease comprises a sequence encoding one or more nuclear localization sequences (NLSs) proximal to an N- or C-terminus of said endonuclease. In some embodiments, said NLS comprises a sequence selected from SEQ ID NOs: 630-645. In some embodiments, said NLS comprises SEQ ID NO: 631. In some embodiments, said NLS is proximal to said N-terminus of said endonuclease. In some embodiments, said NLS comprises SEQ ID NO: 630. In some embodiments, said NLS is proximal to said C-terminus of said endonuclease. In some embodiments, said organism is prokaryotic, bacterial, eukaryotic, fungal, plant, mammalian, rodent, or human.

In some aspects, the present disclosure provides for an engineered vector comprising a nucleic acid sequence encoding a class 2, type V Cas endonuclease, wherein said endonuclease is derived from an uncultivated microorganism.

In some aspects, the present disclosure provides for an engineered vector comprising any nucleic acid disclosed herein. In some embodiments, the vector is a plasmid, a minicircle, a CELiD, an adeno-associated virus (AAV) derived virion, a lentivirus, or an adenovirus.

In some aspects the present disclosure provides for a cell comprising any engineered vector disclosed herein.

In some aspects, the present disclosure provides for a method of manufacturing an endonuclease, comprising cultivating any cell disclosed herein.

In some aspects, the present disclosure provides for a method for binding, cleaving, marking, or modifying a double-stranded deoxyribonucleic acid polynucleotide, comprising: contacting said double-stranded deoxyribonucleic acid polynucleotide with a class 2, type V Cas endonuclease in complex with an engineered guide RNA configured to bind to said endonuclease and said double-stranded deoxyribonucleic acid polynucleotide; wherein said double-stranded deoxyribonucleic acid polynucleotide comprises a protospacer adjacent motif (PAM); and wherein said guide RNA structure comprises a sequence at least 80%, or 90% identical to the non-degenerate nucleotides of any one of SEQ ID NOs: 410-419. In some embodiments, said double-stranded deoxyribonucleic acid polynucleotide comprises a first strand comprising a sequence complementary to a sequence of said engineered guide RNA and a second strand comprising said PAM. In some embodiments, said PAM is directly adjacent to the 5′ end of said sequence complementary to said sequence of said engineered guide RNA. In some embodiments, said PAM comprises a sequence of any one of SEQ ID NOs: 433, 435, 437, 439, 441, 443, 445, 447, 449, 451, 453, 455, 457, 459, 461, 463, 465, 467, 469, 471, 473, and 475. In some embodiments, said class 2, type V Cas endonuclease is derived from an uncultivated microorganism. In some embodiments, said class 2, type V Cas endonuclease further comprises a PAM interacting domain. In some embodiments, said double-stranded deoxyribonucleic acid polynucleotide is a eukaryotic, plant, fungal, mammalian, rodent, or human double-stranded deoxyribonucleic acid polynucleotide.

In some aspects, the present disclosure provides a method of modifying a target nucleic acid locus, said method comprising delivering to said target nucleic acid locus said engineered nuclease system of any one of claims 1-29, wherein said endonuclease is configured to form a complex with said engineered guide ribonucleic acid structure, and wherein said complex is configured such that upon binding of said complex to said target nucleic acid locus, said complex modifies said target nucleic acid locus. In some embodiments, modifying said target nucleic acid locus comprises binding, nicking, cleaving, or marking said target nucleic acid locus. In some embodiments, said target nucleic acid locus comprises deoxyribonucleic acid (DNA) or ribonucleic acid (RNA). In some embodiments, said target nucleic acid comprises genomic DNA, viral DNA, viral RNA, or bacterial DNA. In some embodiments, said target nucleic acid locus is in vitro. In some embodiments, said target nucleic acid locus is within a cell. In some embodiments, said cell is a prokaryotic cell, a bacterial cell, a eukaryotic cell, a fungal cell, a plant cell, an animal cell, a mammalian cell, a rodent cell, a primate cell, a human cell, or a primary cell. In some embodiments, said cell is a primary cell. In some embodiments, said primary cell is a T cell. In some embodiments, said primary cell is a hematopoietic stem cell (HSC). In some embodiments, delivering said engineered nuclease system to said target nucleic acid locus comprises delivering any nucleic acid as disclosed herein or any vector as disclosed herein. In some embodiments, delivering said engineered nuclease system to said target nucleic acid locus comprises delivering a nucleic acid comprising an open reading frame encoding said endonuclease. In some embodiments, said nucleic acid comprises a promoter to which said open reading frame encoding said endonuclease is operably linked. In some embodiments, delivering said engineered nuclease system to said target nucleic acid locus comprises delivering a capped mRNA containing said open reading frame encoding said endonuclease. In some embodiments, delivering said engineered nuclease system to said target nucleic acid locus comprises delivering a translated polypeptide. In some embodiments, delivering said engineered nuclease system to said target nucleic acid locus comprises delivering a deoxyribonucleic acid (DNA) encoding said engineered guide RNA operably linked to a ribonucleic acid (RNA) pol III promoter. In some embodiments, said endonuclease induces a single-stranded break or a double-stranded break at or proximal to said target locus. In some embodiments, said endonuclease induces a staggered single stranded break within or 3′ to said target locus.

In some aspects, the present disclosure provides a host cell comprising an open reading frame encoding a heterologous endonuclease having at least 75% sequence identity to any one of SEQ ID NOs: 1-325, 420-431, 476-624, or 629 or a variant thereof. In some embodiments, said endonuclease has at least 75% sequence identity to any one of SEQ ID NOs: 1, 6, 15, 30, 151, 292, or 319, or a variant thereof. In some embodiments, said endonuclease has at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 30-33, 39, 48, 56, 57, 61, 83, 92, 100, 110, 124, 136, 145, 148, 424, 425, 429, 476, or 629. In some embodiments, said host cell is an E. coli cell. In some embodiments, said E. coli cell is a λDE3 lysogen or said E. coli cell is a BL21(DE3) strain. In some embodiments, said E. coli cell has an ompT lon genotype. In some embodiments, said open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof. In some embodiments, said open reading frame comprises a sequence encoding an affinity tag linked in-frame to a sequence encoding said endonuclease. In some embodiments, said affinity tag is an immobilized metal affinity chromatography (IMAC) tag. In some embodiments, said IMAC tag is a polyhistidine tag. In some embodiments, said affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, said affinity tag is linked in-frame to said sequence encoding said endonuclease via a linker sequence encoding a protease cleavage site. In some embodiments, said protease cleavage site is a tobacco etch virus (TEV) protease cleavage site, a PreScission® protease (PSP) cleavage site, a Thrombin cleavage site, a Factor Xa cleavage site, an enterokinase cleavage site, or any combination thereof. In some embodiments, said open reading frame is codon-optimized for expression in said host cell. In some embodiments, said open reading frame is provided on a vector. In some embodiments, said open reading frame is integrated into a genome of said host cell.

In some aspects, the present disclosure provides a culture comprising any host cell disclosed herein in compatible liquid medium.

In some aspects, the present disclosure provides a method of producing an endonuclease, comprising cultivating any host cell disclosed herein in compatible growth medium. In some embodiments, the method further comprises inducing expression of said endonuclease by addition of an additional chemical agent or an increased amount of a nutrient. In some embodiments, the method further comprises isolating said host cell after said cultivation and lysing said host cell to produce a protein extract. In some embodiments, the method further comprises subjecting said protein extract to IMAC, or ion-affinity chromatography. In some embodiments, the method further comprises cleaving said IMAC affinity tag by contacting a protease corresponding to said protease cleavage site to said endonuclease. In some embodiments, the method further comprises performing subtractive IMAC affinity chromatography to remove said affinity tag from a composition comprising said endonuclease.

In some aspects, the present disclosure provides a method of disrupting a locus in a cell, comprising contacting to said cell a composition comprising: a class 2, type V Cas endonuclease having at least 75% identity to any one of SEQ ID NOs: 1-325, 420-431, 476-624, or 629 or a variant thereof; and an engineered guide RNA, wherein said engineered guide RNA is configured to form a complex with said endonuclease and said engineered guide RNA comprises a spacer sequence configured to hybridize to a region of said locus, wherein said class 2, type V Cas endonuclease has at least equivalent cleavage activity to spCas9 in said cell. In some embodiments, said cleavage activity is measured in vitro by introducing said endonucleases alongside compatible guide RNAs to cells comprising said target nucleic acid and detecting cleavage of said target nucleic acid sequence in said cells. In some embodiments, said composition comprises 20 picomoles (pmol) or less of said class 2, type V Cas endonuclease. In some embodiments, said composition comprises 1 pmol or less of said class 2, type V Cas endonuclease.

In some aspects, the present disclosure provides for a method of disrupting an albumin locus in a cell, comprising contacting to said cell a composition comprising: an endonuclease having at least 75% identity to any one of SEQ ID NOs: 1-325, 420-431, 476-624, or 629 or a variant thereof; and an engineered guide RNA, wherein said engineered guide RNA is configured to form a complex with said endonuclease, and said engineered guide RNA comprises a spacer sequence configured to hybridize to a region of said locus, wherein said engineered guide RNA is configured to hybridize to the any one of the target sequences in Table 6. In some embodiments, said engineered guide RNA comprises a sequence having at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to at least 18 non-degenerate nucleotides of any one of SEQ ID NOs: 414-419432, 434, 436, 438, 440, 442, 444, 446, 448, 450, 452, 454, 456, 458, 460, 462, 464, 466, 468, 470, 472, and 474. In some embodiments, said engineered guide RNA comprises the modified nucleotides of any of the single guide RNA (sgRNA) sequences in Table 6. In some embodiments, said endonuclease has at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 30-33, 39, 48, 56, 57, 61, 83, 92, 100, 110, 124, 136, 145, 148, 424, 425, 429, 476, or 629. In some embodiments, said endonuclease has at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 57. In some embodiments, said region is 5′ to a PAM sequence comprising any one of SEQ ID NOs: 433, 435, 437, 439, 441, 443, 445, 447, 449, 451, 453, 455, 457, 459, 461, 463, 465, 467, 469, 471, 473, and 475.

In some aspects, the present disclosure provides for an isolated RNA molecule comprising a sequence at least about 80%, at least about 85%, at least about 90%, at least about 91%, at least about 92%, at least about 93%, at least about 94%, at least about 95%, at least about 96%, at least about 97%, at least about 98%, at least about 99%, or 100% sequence identity to any sequence in Table 6. In some embodiments, the isolated RNA molecule further comprises the pattern of chemical modifications recited in any of the guide RNAs recited in Table 6.

In some aspects, the present disclosure provides for a use of any RNA molecule disclosed herein for modifying an albumin locus of a cell.

In some aspects, the present disclosure provides for an engineered nuclease system comprising, an endonuclease configured to be selective for a protospacer adjacent motif (PAM) comprising any one of SEQ ID NOs: 433, 435, 437, 439, 441, 443, 445, 447, 449, 451, 453, 455, 457, 459, 461, 463, 465, 467, 469, 471, 473, and 475; and an engineered guide RNA, wherein said engineered guide RNA is configured to form a complex with said endonuclease, and said engineered guide RNA comprises a spacer sequence configured to hybridize to a target nucleic acid sequence. In some embodiments, said endonuclease is a class 2, type V Cas endonuclease. In some embodiments, said endonuclease is not a Cas12a nuclease. In some embodiments, said endonuclease is derived from an uncultivated organism. In some embodiments, said endonuclease further comprises a PAM interacting domain configured to interact with said PAM. In some embodiments, said endonuclease has at least 75% sequence identity to any one of SEQ ID NOs: 1-325, 420-431, 476-624, or 629 or a variant thereof. In some embodiments, said endonuclease has at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 30-33, 39, 48, 56, 57, 61, 83, 92, 100, 110, 124, 136, 145, 148, 424, 425, 429, 476, or 629.

In some aspects, the present disclosure provides an engineered nuclease system comprising: an endonuclease having at least 75% sequence identity to any one of SEQ ID NOs: 1-325, 420-431, 476-624, or 629 or a variant thereof; and a DNA methyltransferase. In some embodiments, said endonuclease has at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 30-33, 39, 48, 56, 57, 61, 83, 92, 100, 110, 124, 136, 145, 148, 424, 425, 429, 476, or 629. In some embodiments, said DNA methyltransferase binds non-covalently to said endonuclease. In some embodiments, said DNA methyltransferase is fused to said endonuclease in a single polypeptide. In some embodiments, said DNA methyltransferase comprises Dmnt3A or Dnmt3L. In some embodiments, said KRAB domain binds non-covalently to said endonuclease or said DNA methyltransferase.

In some embodiments, said KRAB domain is covalently linked to said endonuclease or said DNA methyltransferase. In some embodiments, said KRAB domain is fused to said endonuclease or said DNA methyltransferase in a single polypeptide. In some embodiments, said endonuclease is a nickase or is catalytically dead. In some embodiments, the engineered nuclease system further comprises an engineered guide RNA structure configured to form a complex with said endonuclease, and wherein said engineered guide RNA structure comprises a spacer sequence configured to hybridize to a target nucleic acid sequence. In some embodiments, said target nucleic acid sequence is comprised in or proximal to a promoter of a target genome. In some embodiments, said engineered guide RNA structure comprises one or more: (a) 2′-O-methylnucleotide(s); (b) 2′-fluoronucleotide(s); or (c) phosphorothioate bond(s). In some embodiments, said engineered guide RNA structure comprises the pattern of chemically modified nucleotides of any of the single guide RNAs in Table 6.

In some aspects, the present disclosure provides for a method of modifying a target nucleic acid locus, said method comprising delivering to said target nucleic acid locus any engineered nuclease system disclosed herein, wherein said endonuclease is configured to forma complex with said engineered guide RNA structure, and wherein said complex is configured that upon binding of said complex to said target nucleic acid locus, said DNA methyltransferase modifies said target nucleic acid locus.

In some aspects, the present disclosure provides for a use any engineered nuclease system disclosed herein for modifying a nucleic acid locus. In some embodiments, modifying said nucleic acid locus comprises methylating or demethylating a nucleotide of said nucleic acid locus.

In some aspects, the present disclosure provides for an engineered nuclease system comprising: (a) an endonuclease comprising a RuvC domain, wherein the endonuclease is derived from an uncultivated microorganism, and wherein the endonuclease is not a Cas12a endonuclease; and (b) an engineered guide RNA, wherein the engineered guide RNA is configured to form a complex with the endonuclease and the engineered guide RNA comprises a spacer sequence configured to hybridize to a target nucleic acid sequence. In some aspects, the present disclosure provides an engineered nuclease system comprising: (a) an endonuclease having at least 75% sequence identity to any one of SEQ ID NOs: 1-325, 420-431, 476-624, or 629 or a variant thereof; and (b) an engineered guide RNA, wherein the engineered guide RNA is configured to form a complex with the endonuclease and the engineered guide RNA comprises a spacer sequence configured to hybridize to a target nucleic acid sequence. In some embodiments, the endonuclease comprises a RuvCI, II, or III domain. In some embodiments, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to a RuvCI, II, or III domain of any one of SEQ ID NOs: 1-325, 420-431, 476-624, or 629 or a variant thereof. In some embodiments, the RuvCI domain comprises a D catalytic residue. In some embodiments the RuvCII domain comprises an E catalytic residue. In some embodiments the RuvCIII domain comprises a D catalytic residue. In some embodiments, the RuvC domain does not have nuclease activity. In some embodiments, the endonuclease further comprises a WED II domain having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to a WED II domain of any one of SEQ ID NOs: 1-325, 420-431, 476-624, or 629 or a variant thereof. In some embodiments, the guide RNA comprises a sequence with at least 80% sequence identity to the non-degenerate nucleotides of any one of SEQ ID NOs: 410-419. In some aspects, the present disclosure provides an engineered nuclease system comprising: (a) an engineered guide RNA comprising a sequence with at least 80% sequence identity to the non-degenerate nucleotides of any one of SEQ ID NOs: 410-419, and (b) a class 2, type V Cas endonuclease configured to bind to the engineered guide RNA. In some embodiments, the guide RNA comprises a sequence complementary to a eukaryotic, fungal, plant, mammalian, or human genomic polynucleotide sequence. In some embodiments, the guide RNA is 30-250 nucleotides in length. In some embodiments, the endonuclease comprises one or more nuclear localization sequences (NLSs) proximal to an N- or C-terminus of the endonuclease. In some embodiments, the NLS comprises a sequence at least 80% identical to a sequence from the group consisting of SEQ ID NO: 630-645.

In some embodiments, the engineered nuclease system further comprises a single- or double-stranded DNA repair template comprising from 5′ to 3′: a first homology arm comprising a sequence of at least 20 nucleotides 5′ to the target deoxyribonucleic acid sequence, a synthetic DNA sequence of at least 10 nucleotides, and a second homology arm comprising a sequence of at least 20 nucleotides 3′ to the target sequence. In some embodiments, the first or second homology arm comprises a sequence of at least 40, 80, 120, 150, 200, 300, 500, or 1,000 nucleotides. In some embodiments, the first and second homology arms are homologous to a genomic sequence of a prokaryote, bacteria, fungus, or eukaryote. In some embodiments, the single- or double-stranded DNA repair template comprises a transgene donor. In some embodiments, the engineered nuclease system further comprises a DNA repair template comprising a double-stranded DNA segment flanked by one or two single-stranded DNA segments. In some embodiments, the single-stranded DNA segments are conjugated to the 5′ ends of the double-stranded DNA segment. In some embodiments, the single stranded DNA segments are conjugated to the 3′ ends of the double-stranded DNA segment. In some embodiments, the single-stranded DNA segments have a length from 4 to 10 nucleotide bases. In some embodiments, the single-stranded DNA segments have a nucleotide sequence complementary to a sequence within the spacer sequence. In some embodiments, the double-stranded DNA sequence comprises a barcode, an open reading frame, an enhancer, a promoter, a protein-coding sequence, a miRNA coding sequence, an RNA coding sequence, or a transgene.

In some embodiments, the double-stranded DNA sequence is flanked by a nuclease cut site. In some embodiments, the nuclease cut site comprises a spacer and a PAM sequence. In some embodiments, the system further comprises a source of Mg²⁺. In some embodiments, the guide RNA comprises a hairpin comprising at least 8, at least 10, or at least 12 base-paired ribonucleotides. In some embodiments, the hairpin comprises 10 base-paired ribonucleotides. In some embodiments, a) the endonuclease comprises a sequence at least 75%, 80%, or 90% identical to any one of SEQ ID NOs: 1, 6, 15, 30, 151, 292, or 319, or a variant thereof; and b) the guide RNA structure comprises a sequence at least 80%, or 90% identical to the non-degenerate nucleotides of any one of SEQ ID NOs: 410-419. In some embodiments, the sequence identity is determined by a BLASTP, CLUSTALW, MUSCLE, MAFFT algorithm, or a CLUSTALW algorithm with the Smith-Waterman homology search algorithm parameters. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides an engineered guide RNA comprising: a) a DNA-targeting segment comprising a nucleotide sequence that is complementary to a target sequence in a target DNA molecule; and b) a protein-binding segment comprising two complementary stretches of nucleotides that hybridize to form a double-stranded RNA (dsRNA) duplex, wherein the two complementary stretches of nucleotides are covalently linked to one another with intervening nucleotides, and wherein the engineered guide ribonucleic acid polynucleotide is capable of forming a complex with an endonuclease having at least 75% sequence identity to any one of SEQ ID NOs: 1-325, 420-431, 476-624, or 629, and targeting the complex to the target sequence of the target DNA molecule. In some embodiments, the DNA-targeting segment is positioned 3′ of both of the two complementary stretches of nucleotides. In some embodiments, the protein binding segment comprises a sequence having at least 70%, at least 80%, or at least 90% identity to the non-degenerate nucleotides of SEQ ID NO: 410-419. In some embodiments, the double-stranded RNA (dsRNA) duplex comprises at least 5, at least 8, at least 10, or at least 12 ribonucleotides.

In some aspects, the present disclosure provides a deoxyribonucleic acid polynucleotide encoding an engineered guide ribonucleic acid polynucleotide described herein.

In some aspects, the present disclosure provides a nucleic acid comprising an engineered nucleic acid sequence optimized for expression in an organism, wherein the nucleic acid encodes a class 2, type V Cas endonuclease, and wherein the endonuclease is derived from an uncultivated microorganism, wherein the organism is not the uncultivated organism. In some embodiments, the endonuclease comprises a variant having at least 70% or at least 80% sequence identity to any one of SEQ ID NOs: 1-325, 420-431, 476-624, or 629. In some embodiments, the endonuclease comprises a sequence encoding one or more nuclear localization sequences (NLSs) proximal to an N- or C-terminus of the endonuclease. In some embodiments, the NLS comprises a sequence selected from SEQ ID NOs: 630-645. In some embodiments, the NLS comprises SEQ ID NO: 631. In some embodiments, the NLS is proximal to the N-terminus of the endonuclease. In some embodiments, the NLS comprises SEQ ID NO: 630. In some embodiments, the NLS is proximal to the C-terminus of the endonuclease. In some embodiments, the organism is prokaryotic, bacterial, eukaryotic, fungal, plant, mammalian, rodent, or human.

In some aspects, the present disclosure provides an engineered vector comprising a nucleic acid sequence encoding a class 2, type V Cas endonuclease, wherein the endonuclease is derived from an uncultivated microorganism.

In some aspects, the present disclosure provides an engineered vector comprising a nucleic acid described herein.

In some aspects, the present disclosure provides an engineered vector comprising a deoxyribonucleic acid polynucleotide described herein. In some embodiments, the vector is a plasmid, a minicircle, a CELiD, an adeno-associated virus (AAV) derived virion, a lentivirus, or an adenovirus.

In some aspects, the present disclosure provides a cell comprising a vector described herein.

In some aspects, the present disclosure provides a method of manufacturing an endonuclease, comprising cultivating any of the host cells described herein.

In some aspects, the present disclosure provides a method for binding, cleaving, marking, or modifying a double-stranded deoxyribonucleic acid polynucleotide, comprising: (a) contacting the double-stranded deoxyribonucleic acid polynucleotide with a class 2, type V Cas endonuclease in complex with an engineered guide RNA configured to bind to the endonuclease and the double-stranded deoxyribonucleic acid polynucleotide; wherein the double-stranded deoxyribonucleic acid polynucleotide comprises a protospacer adjacent motif (PAM); and wherein the guide RNA structure comprises a sequence at least 80%, or 90% identical to the non-degenerate nucleotides of any one of SEQ ID NOs: 410-419. In some embodiments, the double-stranded deoxyribonucleic acid polynucleotide comprises a first strand comprising a sequence complementary to a sequence of the engineered guide RNA and a second strand comprising the PAM. In some embodiments, the PAM is directly adjacent to the 5′ end of the sequence complementary to the sequence of the engineered guide RNA. In some embodiments, the class 2, type V Cas endonuclease is derived from an uncultivated microorganism. In some embodiments, the double-stranded deoxyribonucleic acid polynucleotide is a eukaryotic, plant, fungal, mammalian, rodent, or human double-stranded deoxyribonucleic acid polynucleotide.

In some aspects, the present disclosure provides a method of modifying a target nucleic acid locus, the method comprising delivering to the target nucleic acid locus the engineered nuclease system described herein, wherein the endonuclease is configured to form a complex with the engineered guide ribonucleic acid structure, and wherein the complex is configured such that upon binding of the complex to the target nucleic acid locus, the complex modifies the target nucleic acid locus. In some embodiments, modifying the target nucleic acid locus comprises binding, nicking, cleaving, or marking the target nucleic acid locus. In some embodiments, the target nucleic acid locus comprises deoxyribonucleic acid (DNA) or ribonucleic acid (RNA). In some embodiments, the target nucleic acid comprises genomic DNA, viral DNA, viral RNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, a human cell, or a primary cell. In some embodiments, the cell is a primary cell. In some embodiments, the primary cell is a T cell. In some embodiments, the primary cell is a hematopoietic stem cell (HSC). In some embodiments, delivering the engineered nuclease system to the target nucleic acid locus comprises delivering a nucleic acid described herein or a vector described herein. In some embodiments, delivering the engineered nuclease system to the target nucleic acid locus comprises delivering a nucleic acid comprising an open reading frame encoding the endonuclease. In some embodiments, the nucleic acid comprises a promoter to which the open reading frame encoding the endonuclease is operably linked. In some embodiments, delivering the engineered nuclease system to the target nucleic acid locus comprises delivering a capped mRNA containing the open reading frame encoding the endonuclease. In some embodiments, delivering the engineered nuclease system to the target nucleic acid locus comprises delivering a translated polypeptide. In some embodiments, delivering the engineered nuclease system to the target nucleic acid locus comprises delivering a deoxyribonucleic acid (DNA) encoding the engineered guide RNA operably linked to a ribonucleic acid (RNA) pol III promoter. In some embodiments, the endonuclease induces a single-stranded break or a double-stranded break at or proximal to the target locus. In some embodiments, the endonuclease induces a staggered single stranded break within or 3′ to the target locus.

In some aspects, the present disclosure provides a host cell comprising an open reading frame encoding a heterologous endonuclease having at least 75% sequence identity to any one of SEQ ID NOs: 1-325, 420-431, 476-624, or 629 or a variant thereof. In some embodiments, the endonuclease has at least 75% sequence identity to any one of SEQ ID NOs: 1, 6, 15, 30, 151, 292, or 319, or a variant thereof. In some embodiments, the host cell is an E. coli cell or a mammalian cell. In some embodiments, the host cell is an E. coli cell. In some embodiments, the E. coli cell is a λDE3 lysogen or the E. coli cell is a BL21(DE3) strain. In some embodiments, the E. coli cell has an ompT lon genotype. In some embodiments, the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof. In some embodiments, the open reading frame comprises a sequence encoding an affinity tag linked in-frame to a sequence encoding the endonuclease. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, the affinity tag is linked in-frame to the sequence encoding the endonuclease via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof. In some embodiments, open reading frame is codon-optimized for expression in the host cell. In some embodiments, the open reading frame is provided on a vector. In some embodiments, the open reading frame is integrated into a genome of the host cell.

In some aspects, the present disclosure provides a culture comprising any of the host cells described herein in compatible liquid medium.

In some aspects, the present disclosure provides a method of producing an endonuclease, comprising cultivating any of the host cells described herein in compatible growth medium. In some embodiments, the method further comprises inducing expression of the endonuclease by addition of an additional chemical agent or an increased amount of a nutrient. In some embodiments, an additional chemical agent or an increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract. In some embodiments, the method further comprises subjecting the protein extract to IMAC, or ion-affinity chromatography. In some embodiments, the open reading frame comprises a sequence encoding an IMAC affinity tag linked in-frame to a sequence encoding the endonuclease. In some embodiments, the IMAC affinity tag is linked in-frame to the sequence encoding the endonuclease via a linker sequence encoding protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving the IMAC affinity tag by contacting a protease corresponding to the protease cleavage site to the endonuclease. In some embodiments, the method further comprises performing subtractive IMAC affinity chromatography to remove the affinity tag from a composition comprising the endonuclease.

In some aspects, the present disclosure provides a method of disrupting a locus in a cell, comprising contacting to the cell a composition comprising: (a) a class 2, type V Cas endonuclease having at least 75% identity to any one of SEQ ID NOs: 1-325, 420-431, 476-624, or 629 or a variant thereof; and (b) an engineered guide RNA, wherein the engineered guide RNA is configured to form a complex with the endonuclease and the engineered guide RNA comprises a spacer sequence configured to hybridize to a region of the locus, wherein the class 2, type V Cas endonuclease has at least equivalent cleavage activity to spCas9 in the cell. In some embodiments, the cleavage activity is measured in vitro by introducing the endonucleases alongside compatible guide RNAs to cells comprising the target nucleic acid and detecting cleavage of the target nucleic acid sequence in the cells. In some embodiments, the composition comprises 20 pmoles or less of the class 2, type V Cas endonuclease. In some embodiments, the composition comprises 1 pmol or less of the class 2, type V Cas endonuclease.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts typical organizations of CRISPR/Cas loci of different classes and types that were previously described before this disclosure.

FIGS. 2A-2D depict an overview of the MG119 Family. FIG. 2A depicts a multiple alignment of MG119 effectors representatives showing domains compositions and conservation of the RuvC catalytic residues critical for function for a double stranded DNA cleavage activity. FIG. 2B depicts a representation of a CRISPR-containing contig with genomic context surrounding the CRISPR array and the Cas effector (example of MG119-1). FIG. 2C depicts folding of the Direct repeat of MG119-1. FIG. 2D depicts a single guide RNA designed for MG119-1.

FIGS. 3A-3C depict an overview of the MG90 Family. FIG. 3A depicts a multiple alignment of MG90 effectors representatives showing domains compositions and conservation of the RuvC catalytic residues critical for function for a double stranded DNA cleavage activity. FIG. 3B depicts a representation of a CRISPR-containing contig with genomic context surrounding the CRISPR array and the Cas effector (example of MG90-5). FIG. 3C depicts folding of the Direct repeat of MG90-5.

FIGS. 4A-4C depict an overview of the MG126 Family. FIG. 4A depicts a multiple alignment of MG126 effectors representatives showing domains compositions and conservation of the RuvC catalytic residues critical for function for a double stranded DNA cleavage activity. FIG. 4B depicts a representation of a CRISPR-containing contig with genomic context surrounding the CRISPR array and the Cas effector (example of MG126-4). FIG. 4C depicts folding of the Direct repeat of MG126-4.

FIGS. 5A-5C depict an overview of the MG118 Family. FIG. 5A depicts a multiple alignment of MG118 effectors representatives showing domains compositions and conservation of the RuvC catalytic residues critical for function for a double stranded DNA cleavage activity. FIG. 5B depicts a representation of a CRISPR-containing contig with genomic context surrounding the CRISPR array and the Cas effector (example of MG118-1). FIG. 5C depicts folding of the Direct repeat of MG118-1.

FIGS. 6A-6C depict an overview of the MG122 Family. FIG. 6A depicts a multiple alignment of MG122 effectors representatives showing domains compositions and conservation of the RuvC catalytic residues critical for function for a double stranded DNA cleavage activity.

FIG. 6B depicts a representation of a CRISPR-containing contig with genomic context surrounding the CRISPR array and the Cas effector (example of MG122-4). FIG. 6C depicts folding of the Direct repeat of MG122-4.

FIGS. 7A-7C depict an overview of the MG120 Family. FIG. 7A depicts a multiple alignment of MG120 effectors representatives showing domains compositions and conservation of the RuvC catalytic residues critical for function for a double stranded DNA cleavage activity. FIG. 7B depicts a representation of a CRISPR-containing contig with genomic context surrounding the CRISPR array and the Cas effector (example of MG120-1). FIG. 7C depicts folding of the Direct repeat of MG120-1.

FIGS. 8A-8D depict an overview of the MG91 Family. FIG. 8A depicts a representation of a CRISPR-containing contig with genomic context surrounding the CRISPR array and the Cas effector (example of MG91B-24). FIG. 8B depicts folding of the Direct repeats of MG91B-24. FIG. 8C depicts a representation of a CRISPR-containing contig with genomic context surrounding the CRISPR array and the Cas effector (example of MG91C-10). FIG. 8D depicts folding of the Direct repeats of MG91C-10.

FIG. 9 depicts in vitro activity of MG119-2 using the TXTL assay. MG119-2 was tested for dsDNA cleavage with two intergenic sequences from the MG119-2 contig, minimal array (MA) sequences containing repeats in the forward or reverse orientation, and a PAM library target plasmid. Positive intergenic enrichment was observed in lane 1 as an amplified cleavage product with intergenic (IG) sequence 1 and the minimal array with repeats in the forward orientation. Lanes 3 and 7 are the negative controls where IGs were omitted, and lane 4 is a third negative control where both the arrays and IGs were omitted.

FIG. 10A depicts a SeqLogo of the MG119-2 PAM (5′-nTnn-3′) determined via next-generation sequencing (NGS) of the cleavage products obtained from the in vitro cleavage assay. FIG. 10B depicts a histogram of the cutsite (23 bd away from the PAM).

FIGS. 11A and 11B depict examples of active MG119 nuclease and their sgRNA designs. FIG. 11A depicts predicted folding for single guide RNA sequences without spacers. The blue circle represents the first 5′ nucleotide of the tracrRNA and the red circle represents the 3′ nucleotide of the repeat. TracrRNA and repeat sequences are looped with a GAAA tetraloop. The repeat anti-repeat fold is on the 3′ end of each structure. Depicted are three different RNA structures of active guides within the same family. From left to right: the MG119-28 guide has four hairpins, three smaller ones on the 5′ end and a very long hairpin with two bulges next to the repeat anti-repeat fold. The MG119-83 sgRNA has three small hairpins and the repeat anti-repeat has two bulges. The MG119-118 has four hairpins, the second hairpin from the 5′ end branches into three hairpins while the third hairpin and the repeat anti-repeat have one bulge. This guide also has some pairing nucleotides between the 5′ end of the tracr and the 3′ end of the repeat. FIG. 11B depicts in vitro cleavage assay amplification products on 2% agarose gels. Low molecular weight DNA ladders (NEB) are in lanes 1, 7, and 11. Other lane contents from left to right: (2) MG119-28 nuclease only, MG119-28 nuclease plus (3) sgRNA1 with U67 spacer, (4) sgRNA1 with U40 spacer, (5) sgRNA2 with U67 spacer, and (6) sgRNA2 with U40 spacer; (8) MG119-83 nuclease only, MG119-83 nuclease plus (9) sgRNA1 with U67 spacer and (10) sgRNA1 with U40 spacer; (12) MG119-118 nuclease only, MG119-118 nuclease plus (13) sgRNA1 with U67 spacer and (14) sgRNA1 with U40 spacer. Resulting amplicon products are 188 bp with a U67 spacer carrying guide or 205 bp with a U40 spacer carrying guide.

FIG. 12 depicts sequence logos of protospacer adjacent motifs (PAMs) for active MG119 nucleases.

FIGS. 13A-13F depict example SDS-PAGE gels of protein purification steps and size exclusion chromatography (SEC) A280 traces. FIG. 13A depicts MG119-28Δ purification with samples recovered (1) post-sonication lysis, (2) post-clarification centrifugation, (3) Ni-NTA gravity column flow-through, (4) eluate from Ni-NTA resin, (5) concentrated sample. FIG. 13B depicts S200i 10/300 GL column SEC A280 trace. Peak fractions were pooled and concentrated. FIGS. 13C and 13D depict MBP-tagged/cleaved MG119-28Δ purification with samples recovered (1) post-sonication lysis, (2) post-clarification centrifugation, (3) Ni-NTA gravity column flow-through, (4) eluate from Ni-NTA resin, (5) concentrated protein, (6) concentrated protein cleaved overnight with TEV protease, (7) and centrifuged (21,000×g, 4° C., 10 min) to pellet aggregates, (8) Amylose column flow-through, (9) centrifuged flow-through (21,000×g, 4° C., 10 min) to pellet aggregate, and (10) concentrated flow-through. FIG. 13E depicts S200i 10/300 GL column SEC A280 trace. FIG. 13F depicts data demonstrating that of the five MG119 candidates expressed in both the pMGB and pMGBA expression vectors, all showed higher yields in the pMGBΔ vector.

FIGS. 14A and 14B depict an example of in vitro cleavage efficiency with purified protein. FIG. 14A depicts an agarose gel showing RNP:substrate ratio titration and increasing substrate cleavage at higher ratios. FIG. 14B depicts the percent of substrate cleaved determined for each lane using densitometry. Cleavage fractions were plotted in Prism8, and the slope of the linear range of cleavage was used to calculate protein active fraction. This assay used MG119-28 expressed in the pMGBΔ backbone.

FIGS. 15A and 15B depict examples of in vitro cleavage and editing efficiency of mouse Hepa1-6 cells DNA. FIG. 15A depicts percent cleavage of MG119-28 with four chemically modified guides targeting the mouse albumin gene at intron 1 (Table 6). Two concentrations of nuclease were tested 15.6 nM (black bars) and 7.8 nM (white bars). Cleavage was normalized to the non-targeting control. MG119-28 can cleave Hepa 1-6 gDNA up to an average of 60% with sgRNA4 at 15.6 nM RNP and up to 33% at 7.8 nM RNP. FIG. 15B depicts percent INDEL generated by MG119-28 in Hepa 1-6 cells normalized to apo reactions. Each condition was performed in triplicate. An average of 25.12% of the sequenced reads were edited with sgRNA3. sgRNA3 is consistently active in vitro and in cells as shown here. The next best guide in cells is sgRNA4 with an average of 4.11% editing. The edits observed are largely a deletion between 4-24 bp.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG122

SEQ ID NOs: 1-5 show the full-length peptide sequences of MG122 nucleases.

### MG120

SEQ ID NOs: 6-14 show the full-length peptide sequences of MG120 nucleases.

SEQ ID NOs: 333-335 and 355-357 show nucleotide sequences of MG120 tracrRNAs derived from the same loci as a MG120 Cas effector.

SEQ ID NOs: 374-375 and 389-390 show nucleotide sequences of MG120 minimal arrays.

### MG118

SEQ ID NO: 15 shows the full-length peptide sequence of an MG118 nuclease.

SEQ ID NO: 376 shows a nucleotide sequence of an MG118 minimal array.

SEQ ID NO: 391 shows a nucleotide sequence of an MG118 minimal array.

SEQ ID NOs: 400-401 show nucleotide sequences of MG118 target CRISPR repeats.

SEQ ID NOs: 410-411 show nucleotide sequences of MG118 crRNAs.

### MG90

SEQ ID NOs: 16-29 show the full-length peptide sequences of MG90 nucleases.

SEQ ID NOs: 346-347 and 368-369 show nucleotide sequences of MG90 tracrRNAs derived from the same loci as a MG90 Cas effector.

SEQ ID NOs: 383-384 and 398-399 show nucleotide sequences of MG90 minimal arrays.

SEQ ID NOs: 402-403 show nucleotide sequences of MG90 target CRISPR repeats.

SEQ ID NOs: 412-413 show nucleotide sequences of MG90 sgRNAs.

### MG119

SEQ ID NOs: 30-150, 420-431, 476-624, and 629 show the full-length peptide sequences of MG119 nucleases.

SEQ ID NOs: 326-332, 336-345, 348-354, and 358-367 show nucleotide sequences of MG119 tracrRNAs derived from the same loci as a MG119 Cas effector.

SEQ ID NOs: 370-373, 377-382, 385-388, and 392-397 show nucleotide sequences of MG119 minimal arrays.

SEQ ID NOs: 404-409 show nucleotide sequences of MG119 target CRISPR repeats.

SEQ ID NOs: 414-419, 432, 434, 436, 438, 440, 442, 444, 446, 448, 450, 452, 454, 456, 458, 460, 462, 464, 466, 468, 470, 472, and 474 show nucleotide sequences of MG119 sgRNAs.

SEQ ID NOs: 433, 435, 437, 439, 441, 443, 445, 447, 449, 451, 453, 455, 457, 459, 461, 463, 465, 467, 469, 471, 473, and 475 show nucleotide sequences of MG119 PAMs.

### MG91B

SEQ ID NOs: 151-291 show the full-length peptide sequences of MG91B nucleases.

### MG91C

SEQ ID NOs: 292-318 show the full-length peptide sequences of MG91C nucleases.

### MG91A

SEQ ID NO: 319 shows the full-length peptide sequence of an MG91A nuclease.

### MG126

SEQ ID NOs: 320-325 show the full-length peptide sequences of MG126 nucleases.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, fems, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [uS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels, and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [RI 10]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Il.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88 (which is entirely incorporated by reference herein).

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic necessary elements to promote transcriptional expression of an operably linked polynucleotide. Eukaryotic basal promoters typically, though not necessarily, contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner known to be attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” can generally be used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

As used herein, the term “Cas12a” generally refers to a family of Cas endonucleases that are class 2, Type V-A Cas endonucleases and that (a) use a relatively small guide RNA (about 42-44 nucleotides) that is processed by the nuclease itself following transcription from the CRISPR array, and (b) cleave DNA to leave staggered cut sites. Further features of this family of enzymes can be found, e.g. in Zetsche B, Heidenreich M, Mohanraju P, et al. Nat Biotechnol 2017; 35:31-34, and Zetsche B, Gootenberg J S, Abudayyeh 00, et al. Cell 2015; 163:759-771, which are incorporated by reference herein.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence” or “spacer sequence.” A nucleic acid-targeting segment may comprise a sub-segment that may be referred to as a “protein binding segment” or “protein binding sequence” or “Cas protein binding segment”.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HMMER hmmalign with default parameters.

The term “optimally aligned” in the context of two or more nucleic acids or polypeptide sequences, generally refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

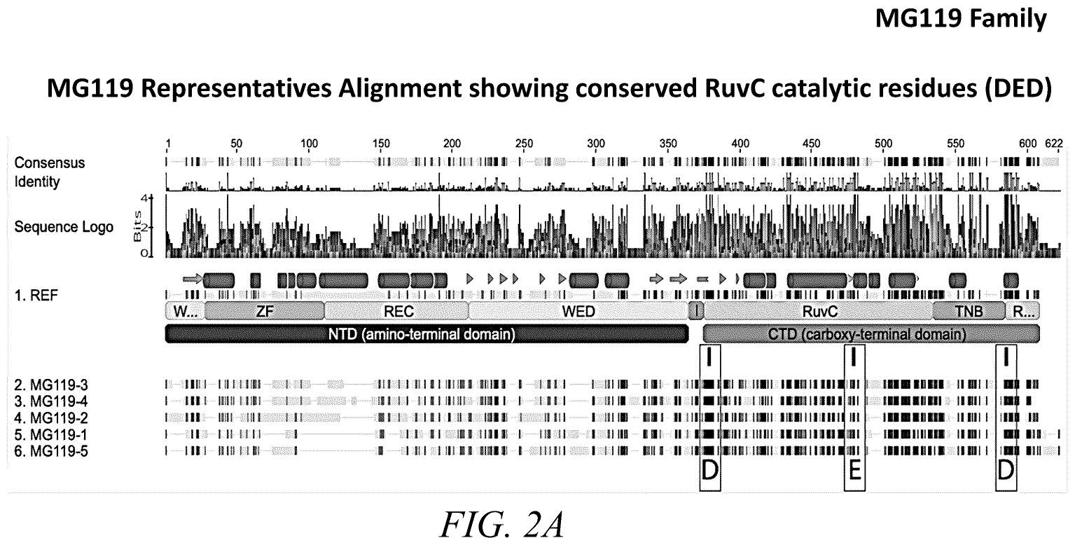

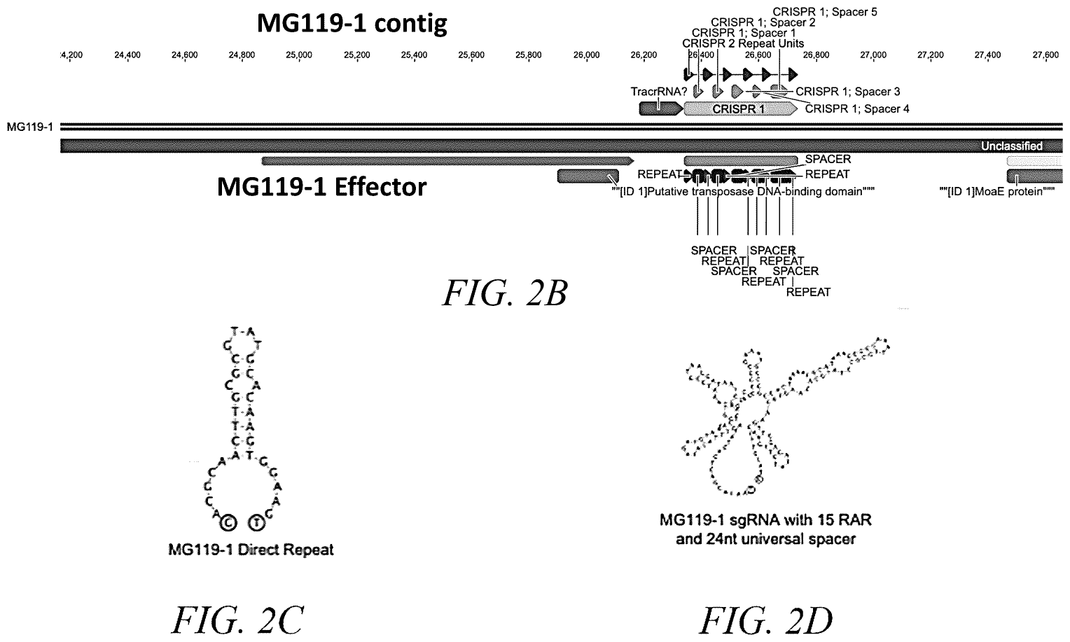

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to any one of the endonuclease protein sequences described herein (e.g. MG90, MG91A, MG91B, MG91C, MG118, MG119, MG120, MG122, or MG126 family endonucleases described herein, or any other family nuclease described herein). In some embodiments, such conservatively substituted variants are functional variants. Such functional variants can encompass sequences with substitutions such that the activity of one or more critical active site residues or guide RNA binding residues of the endonuclease are not disrupted. In some embodiments, a functional variant of any of the proteins described herein lacks substitution of at least one of the conserved or functional residues called out in FIGS. 2A, 3A, 4A, 5A, or 6A. In some embodiments, a functional variant of any of the proteins described herein lacks substitution of all of the conserved or functional residues called out in FIGS. 2A, 3A, 4A, 5A, or 6A.

Also included in the current disclosure are variants of any of the enzymes described herein with substitution of one or more catalytic residues to decrease or eliminate activity of the enzyme (e.g. decreased-activity variants). In some embodiments, a decreased activity variant as a protein described herein comprises a disrupting substitution of at least one, at least two, or all three catalytic residues called out in FIGS. 2A, 3A, 4A, 5A, or 6A.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another: 1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

### Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches containing large numbers of microbial species may offer the potential to drastically increase the number of new CRISPR/Cas systems known and speed the discovery of new oligonucleotide editing functionalities. A recent example of the fruitfulness of such an approach is demonstrated by the 2016 discovery of CasX/CasY CRISPR systems from metagenomic analysis of natural microbial communities.

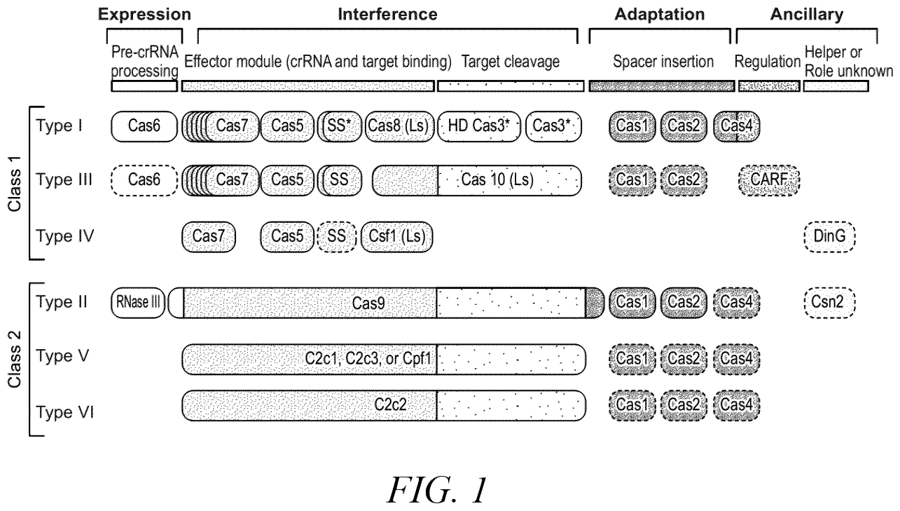

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types, and 16 subtypes based on shared functional characteristics and evolutionary similarity (see FIG. 1).

Class I CRISPR-Cas systems have large, multi-subunit effector complexes, and comprise Types I, III, and IV. Class II CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Cas II nucleases are known as DNA nucleases. Type 2 effectors generally exhibit a structure consisting of a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g. Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are again known as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

CRISPR-Cas systems have emerged in recent years as the gene editing technology of choice due to their targetability and ease of use. The most commonly used systems are the Class 2 Type II SpCas9 and the Class 2 Type V-A Cas12a (previously Cpf1). The Type V-A systems in particular are becoming more widely used since their reported specificity in cells is higher than other nucleases, with fewer or no off-target effects. The V-A systems are also advantageous in that the guide RNA is small (42-44 nucleotides compared with approximately 100 nt for SpCas9) and is processed by the nuclease itself following transcription from the CRISPR array, simplifying multiplexed applications with multiple gene edits. Furthermore, the V-A systems have staggered cut sites, which may facilitate directed repair pathways, such as microhomology-dependent targeted integration (MITI).

The most commonly used Type V-A enzymes require a 5′ protospacer adjacent motif (PAM) next to the chosen target site: 5′-TTTV-3′ for Lachnospiraceae bacterium ND2006 LbCas12a and Acidaminococcus sp. AsCas12a; and 5′-TTV-3′ for Francisella novicida FnCas12a. Recent exploration of orthologs has revealed proteins with less restrictive PAM sequences that are also active in mammalian cell culture, for example YTV, YYN or TTN.

However, these enzymes do not fully encompass Type V biodiversity and targetability, and may not represent all possible activities and PAM sequence requirements. Here, thousands of genomic fragments were mined from numerous metagenomes for Type V nucleases. The known diversity of V enzymes may have been expanded and novel systems may have been developed into highly targetable, compact, and precise gene editing agents.

### MG Enzymes

Type V CRISPR systems are quickly being adopted for use in a variety of genome editing applications. These programmable nucleases are part of adaptive microbial immune systems, the natural diversity of which has been largely unexplored. Novel families of Type V CRISPR enzymes were identified through a large-scale analysis of metagenomes collected from a variety of complex environments, and representatives of these were developed systems into gene-editing platforms. The majority of these systems come from uncultivated organisms, some of which encode a divergent Type V effector within the same CRISPR operon.

In some aspects, the present disclosure provides for novel Type V candidates. These candidates may represent one or more novel subtypes and some sub-families may have been identified. These nucleases are less than about 900 amino acids in length. These novel subtypes may be found in the same CRISPR locus as known Type V effectors. RuvC catalytic residues may have been identified for the novel Type V candidates, and these novel Type V candidates may not require tracrRNA.

In some aspects, the present disclosure provides for smaller Type V effectors. Such effectors may be small putative effectors. These effectors may simplify delivery and may extend therapeutic applications.

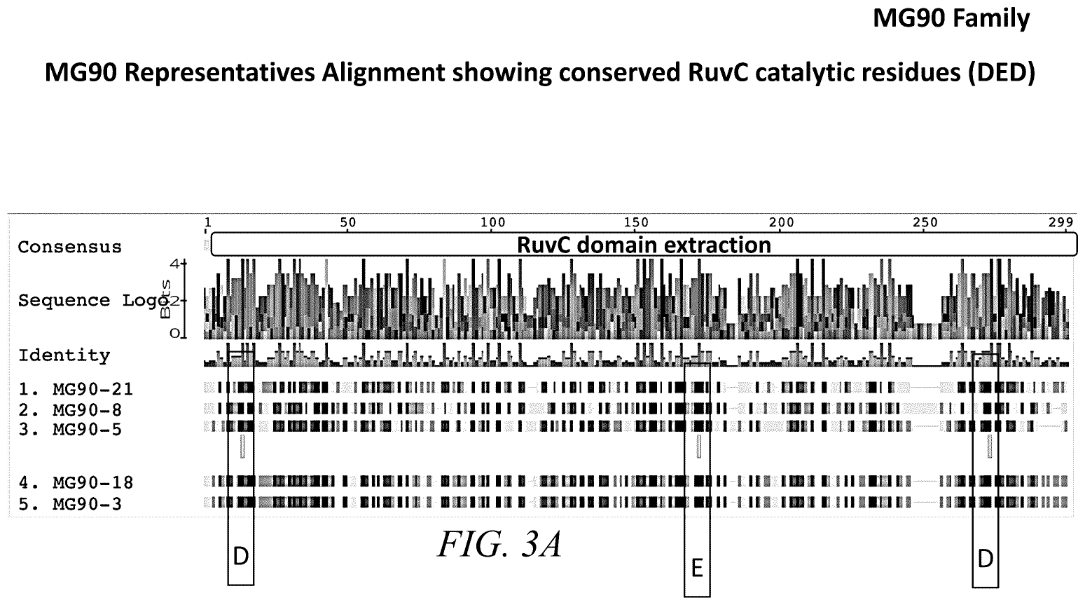

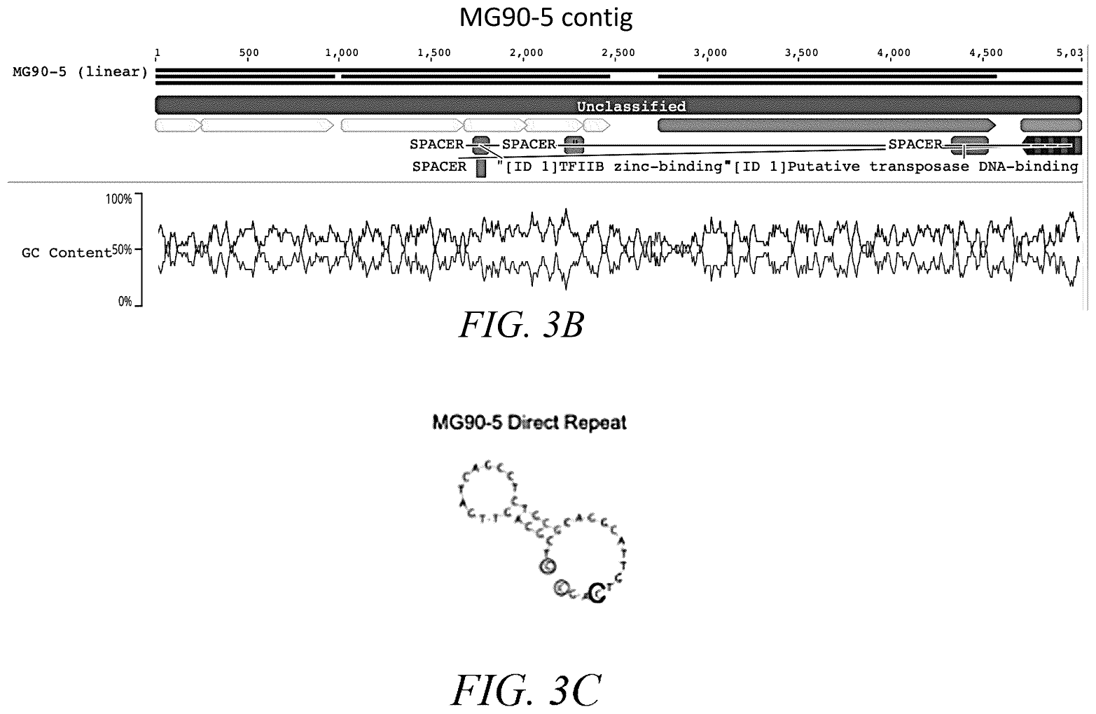

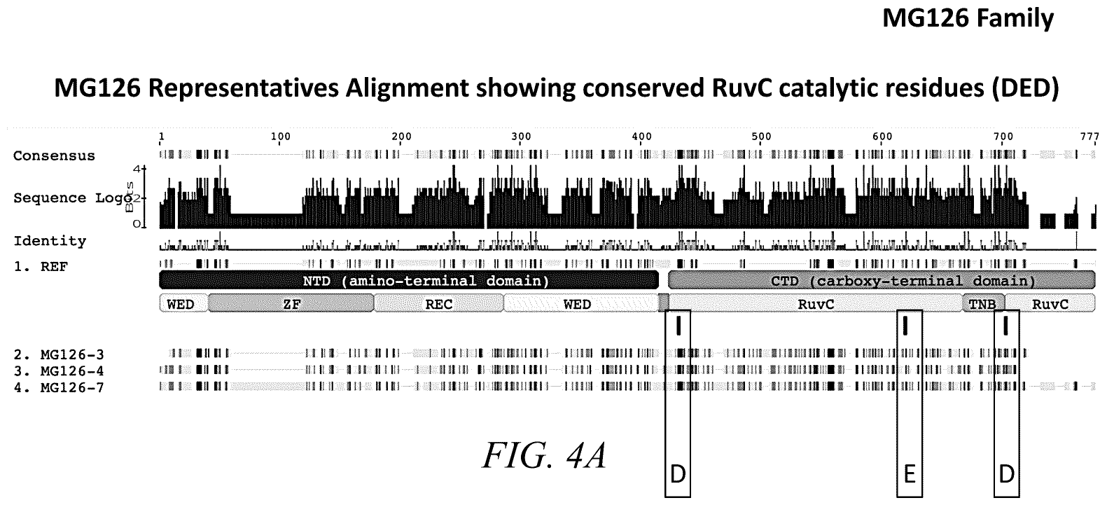

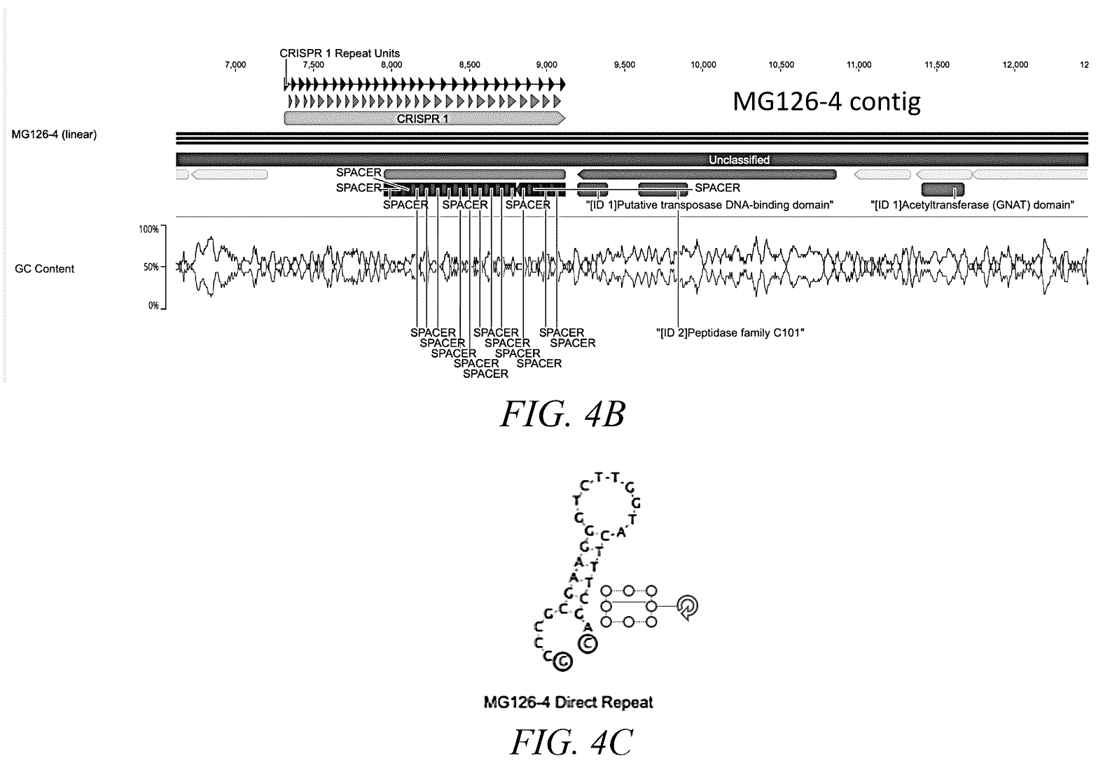

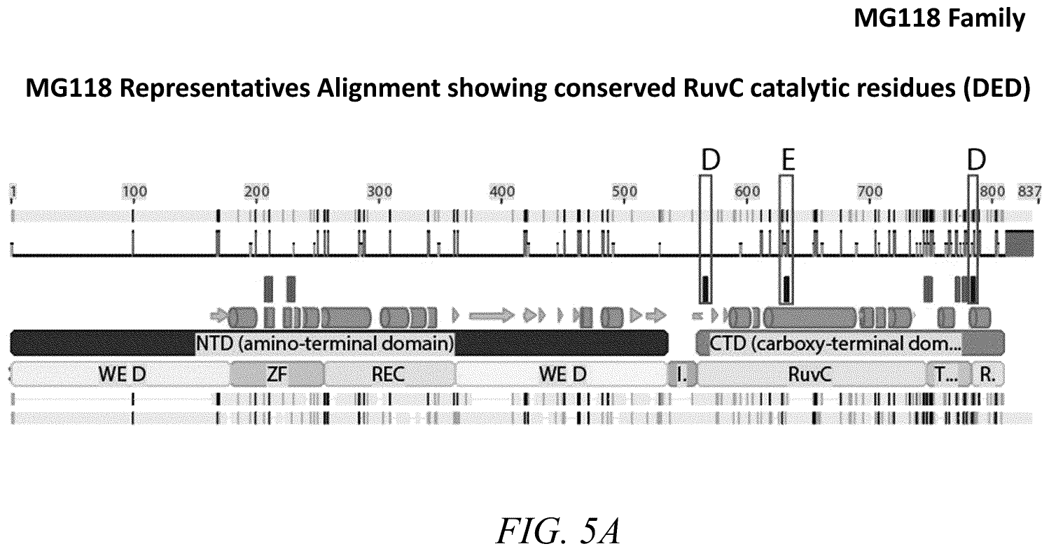

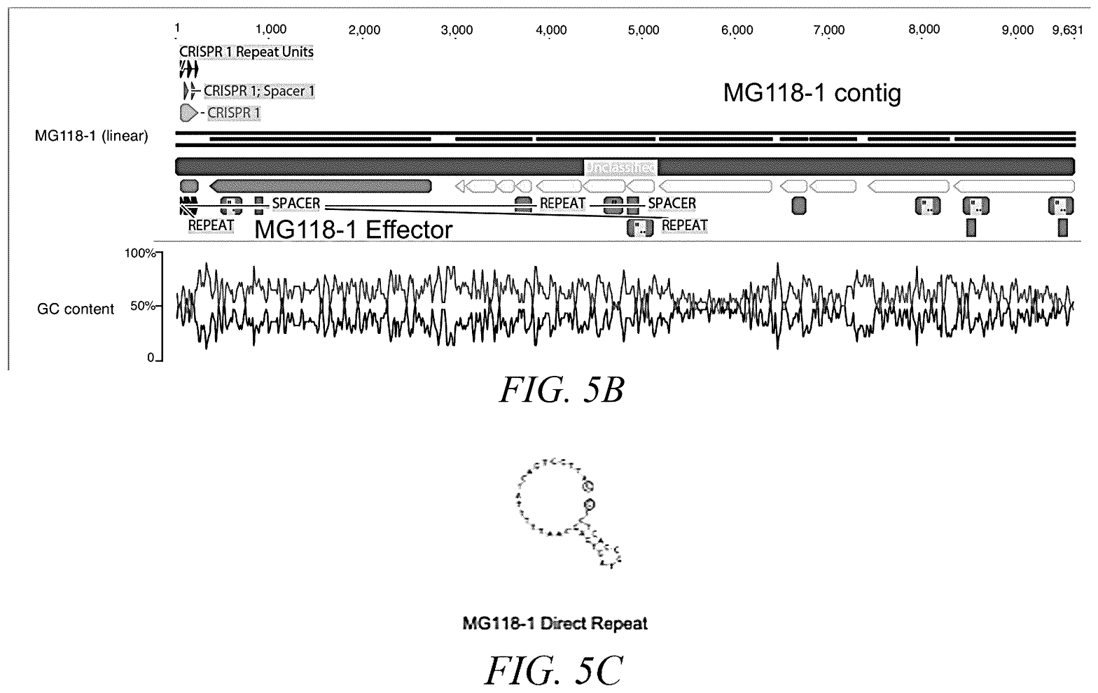

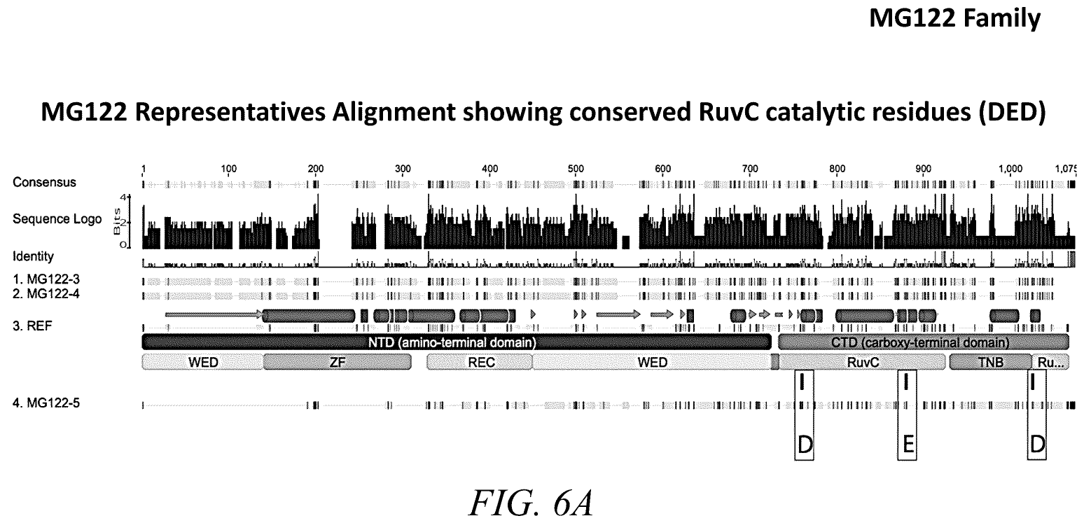

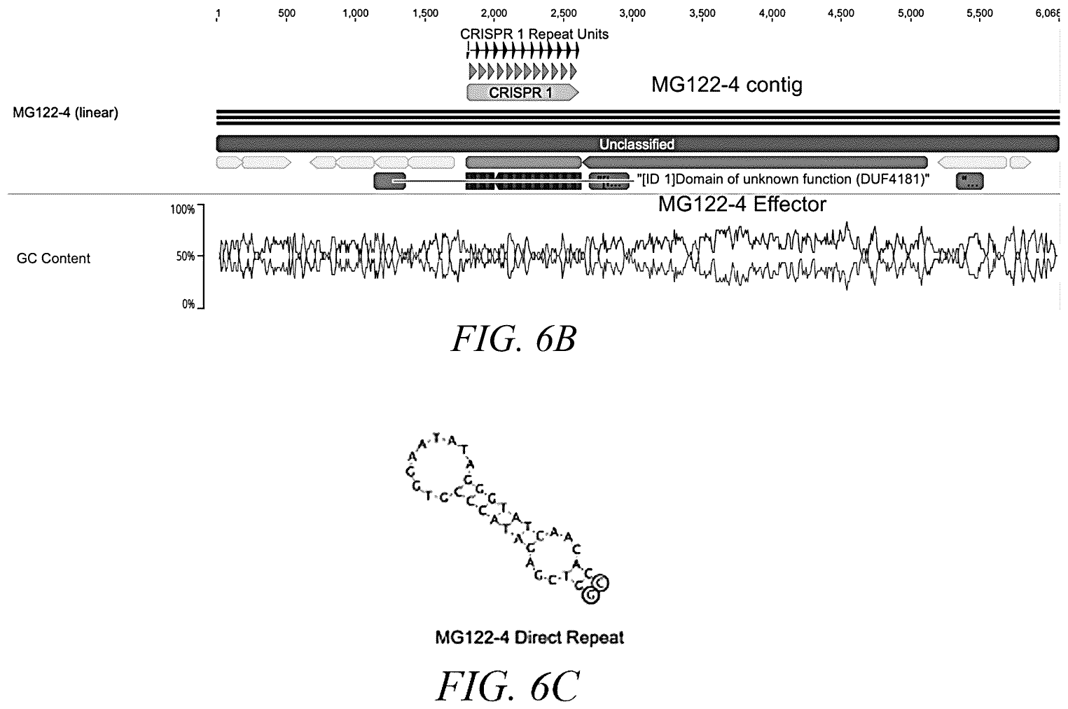

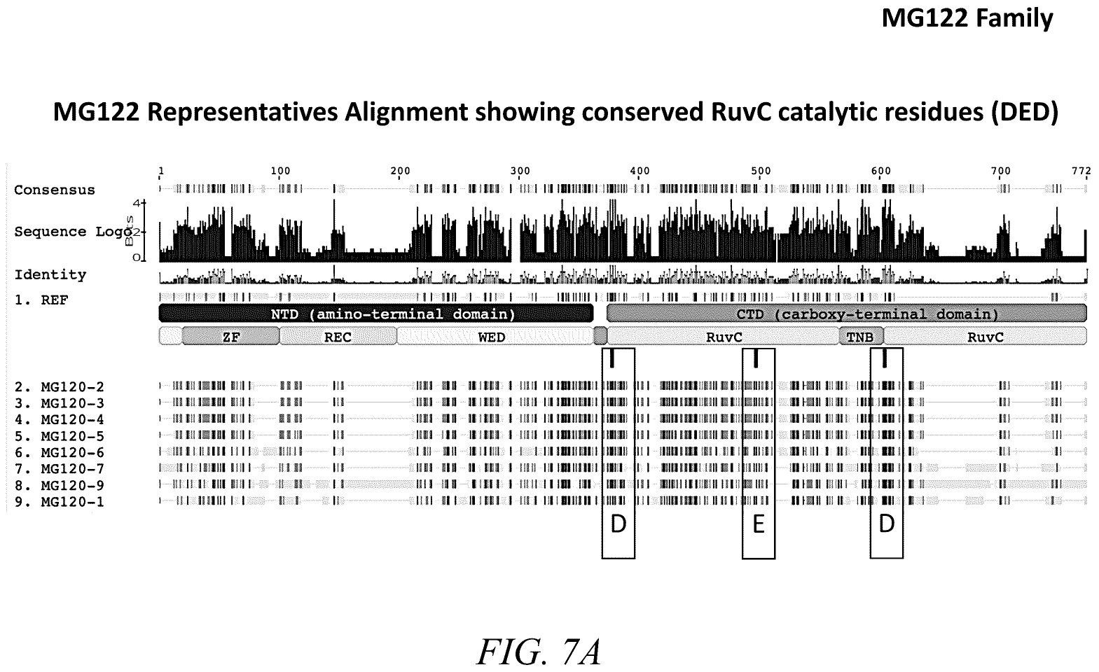

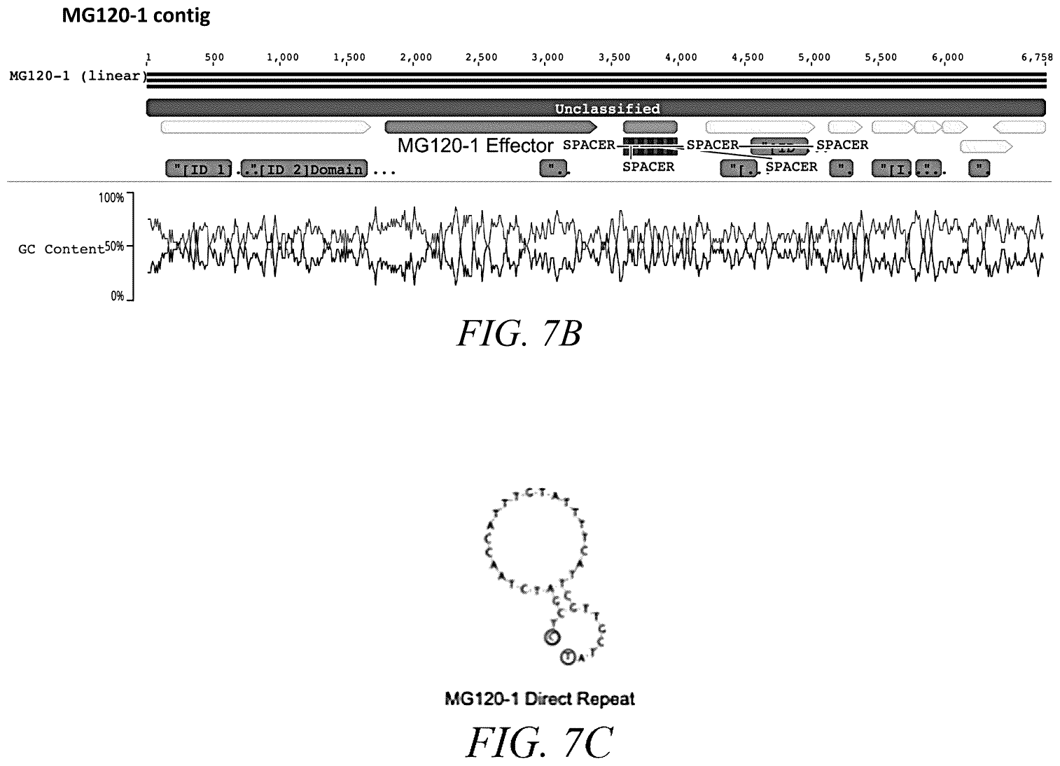

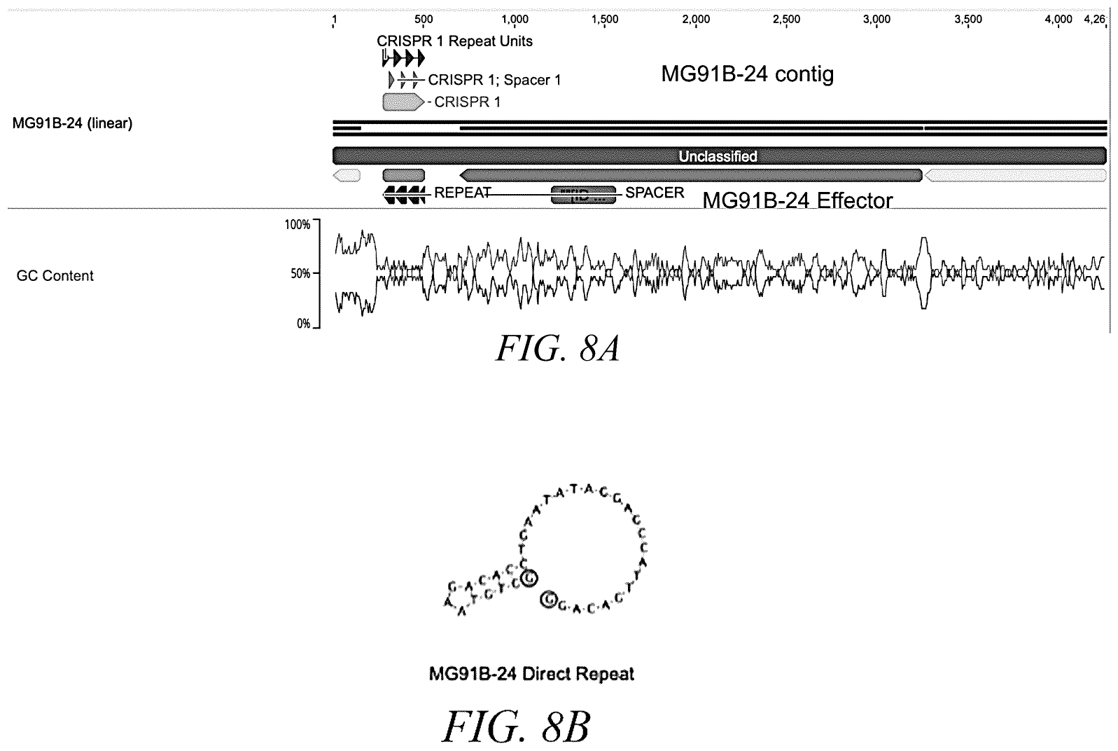

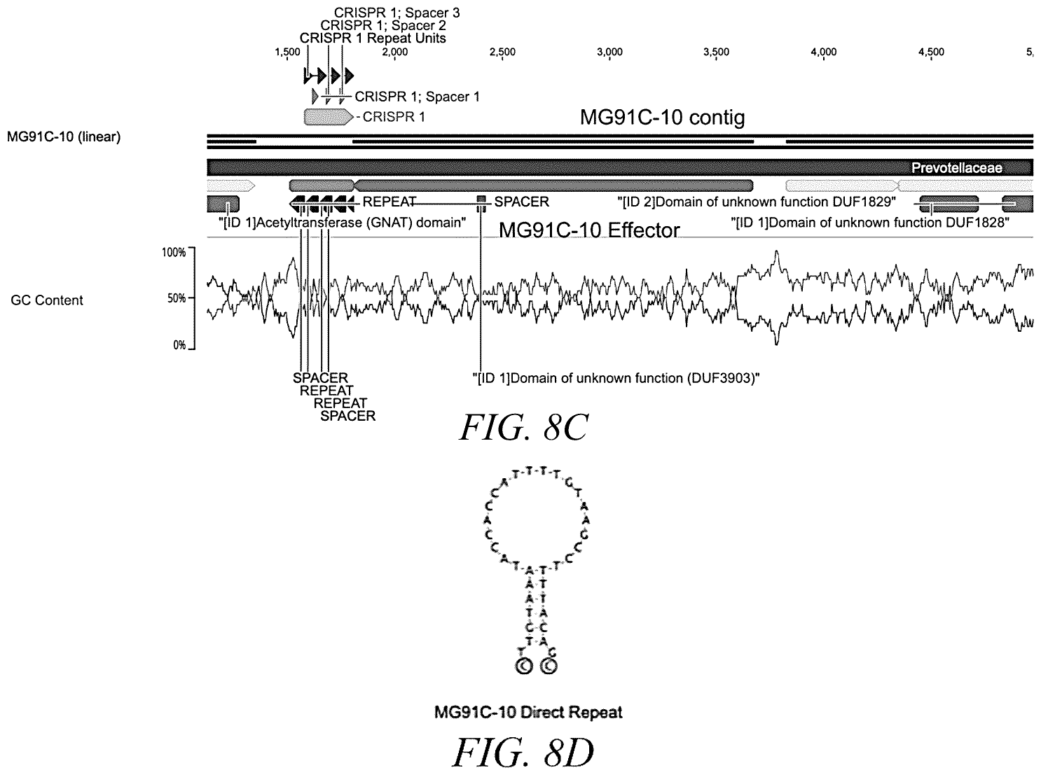

In some aspects, the present disclosure provides for a novel type V effector. Such an effector may be MG90 as described herein (see FIGS. 3A-3C). Such an effector may be MG91 as described herein (see FIGS. 8A-8B). Such an effector may be MG118 as described herein (see FIGS. 5A-5C). Such an effector may be MG119 as described herein (see FIGS. 2A-2D). Such an effector may be MG120 as described herein (see FIGS. 7A-7C). Such an effector may be MG122 as described herein (see FIGS. 6A-6C). Such an effector may be MG126 as described herein (see FIGS. 4A-4C).

In one aspect, the present disclosure provides for an engineered nuclease system discovered through metagenomic sequencing. In some cases, the metagenomic sequencing is conducted on samples. In some cases, the samples may be collected from a variety of environments. Such environments may be a human microbiome, an animal microbiome, environments with high temperatures, environments with low temperatures. Such environments may include sediment.

In one aspect, the present disclosure provides for an engineered nuclease system comprising an endonuclease. In some cases, the endonuclease is a Cas endonuclease. In some cases, the endonuclease is a class 2, type V Cas endonuclease. In some cases, the endonuclease is a class 2, type V Cas endonuclease of a novel sub-type. In some cases, the endonuclease is derived from an uncultivated microorganism. The endonuclease may comprise a RuvC domain. In some cases, the engineered nuclease system comprises an engineered guide RNA. In some cases, the engineered guide RNA is configured to form a complex with the endonuclease. In some cases, the engineered guide RNA comprises a spacer sequence. In some cases, the spacer sequence is configured to hybridize to a target nucleic acid sequence.

In one aspect, the present disclosure provides for an engineered nuclease system comprising an endonuclease. In some cases, the endonuclease has at least about 70% sequence identity to any one of SEQ ID NOs: 1-325, 420-431, 476-624, or 629. In some cases, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-325, 420-431, 476-624, or 629.

In some cases, the endonuclease comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-325, 420-431, 476-624, or 629. In some cases, the endonuclease may be substantially identical to any one of SEQ ID NOs: 1-325, 420-431, 476-624, or 629.

In some cases, the engineered nuclease system comprises an engineered guide RNA. In some cases, the engineered guide RNA is configured to form a complex with the endonuclease. In some cases, the engineered guide RNA comprises a spacer sequence. In some cases, the spacer sequence is configured to hybridize to a target nucleic acid sequence. In some cases, the endonuclease is configured to bind to a protospacer adjacent motif (PAM) sequence.

In some cases, the endonuclease is not a Cpf1 or Cms1 endonuclease.

In some cases, the guide RNA comprises a sequence with at least 80% sequence identity to the first 19 nucleotides or the non-degenerate nucleotides of SEQ ID NO: 410-419. In some cases, the guide RNA comprises a sequence with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 410-419. In some cases, the guide RNA comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 410-419. In some cases, the guide RNA comprises a sequence which is substantially identical to the first 19 nucleotides or the non-degenerate nucleotides of SEQ ID NO: 410-419.

In some cases, the guide RNA comprises a sequence with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to the first 19 nucleotides or the non-degenerate nucleotides of SEQ ID NO: 410-419. In some cases, the endonuclease is configured to bind to the engineered guide RNA. In some cases, the Cas endonuclease is configured to bind to the engineered guide RNA. In some cases, the class 2 Cas endonuclease is configured to bind to the engineered guide RNA. In some cases, the class 2, type V Cas endonuclease is configured to bind to the engineered guide RNA. In some cases, the class 2, type V, novel subtype Cas endonuclease is configured to bind to the engineered guide RNA.

In some cases, the guide RNA comprises a sequence complementary to a eukaryotic, fungal, plant, mammalian, or human genomic polynucleotide sequence. In some cases, the guide RNA comprises a sequence complementary to a eukaryotic genomic polynucleotide sequence. In some cases, the guide RNA comprises a sequence complementary to a fungal genomic polynucleotide sequence. In some cases, the guide RNA comprises a sequence complementary to a plant genomic polynucleotide sequence. In some cases, the guide RNA comprises a sequence complementary to a mammalian genomic polynucleotide sequence. In some cases, the guide RNA comprises a sequence complementary to a human genomic polynucleotide sequence.

In some cases, the guide RNA is 30-250 nucleotides in length. In some cases, the guide RNA is 42-44 nucleotides in length. In some cases, the guide RNA is 42 nucleotides in length. In some cases, the guide RNA is 43 nucleotides in length. In some cases, the guide RNA is 44 nucleotides in length. In some cases, the guide RNA is 85-245 nucleotides in length. In some cases, the guide RNA is more than 90 nucleotides in length. In some cases, the guide RNA is less than 245 nucleotides in length.

In some cases, the endonuclease may comprise a variant having one or more nuclear localization sequences (NLSs). The NLS may be proximal to the N- or C-terminus of the endonuclease. The NLS may be appended N-terminal or C-terminal to any one of SEQ ID NOs: 630-645, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 630-645. In some cases, the NLS may comprise a sequence substantially identical to any one of SEQ ID NOs: 630-645.

TABLE 1
Example NLS Sequences that may be used with
Cas Effectors according to the disclosure.

| Source | NLS amino acid sequence | SEQ ID NO: |
|---|---|---|
| SV40 | PKKKRKV | 630 |
| nucleoplasmin | KRPAATKKAGQAKKKK | 631 |
| bipartite NLS | | |
| c-myc NLS | PAAKRVKLD | 632 |
| c-myc NLS | RQRRNELKRSP | 633 |
| hRNPA1 M9 NLS | NQSSNFGPMKGGNFGGRSSGPYGGGGQYFAKPRNQGGY | 634 |
| Importin-alpha IBB | RMRIZFKNKGKDTAELRRRRVEVSVELRKAKKDEQILKRRNV | 635 |
| domain | | |
| Myoma T protein | VSRKRPRP | 636 |
| Myoma T protein | PPKKARED | 637 |
| p53 | PQPKKKPL | 638 |
| mouse c-ab1 IV | SALIKKKKKMAP | 639 |
| influenza virus NS1 | DRLRR | 640 |
| influenza virus NS1 | PKQKKRK | 641 |
| Hepatitis virus delta | RKLKKKIKKL | 642 |
| antigen | | |
| mouse Mx1 protein | REKKKELKRR | 643 |
| human poly(ADP- | KRKGDEVDGVDEVAKKKSKK | 644 |
| ribose) polymerase | | |
| steroid hormone | RKCLQAGMNLEARKTKK | 645 |
| receptors (human) | | |
| glucocorticoid | | |

In some cases, the engineered nuclease system further comprises a single- or double stranded DNA repair template. In some cases, the engineered nuclease system further comprises a single-stranded DNA repair template. In some cases, the engineered nuclease system further comprises a double-stranded DNA repair template. In some cases, the single- or double-stranded DNA repair template may comprise from 5′ to 3′: a first homology arm comprising a sequence of at least 20 nucleotides 5′ to said target deoxyribonucleic acid sequence, a synthetic DNA sequence of at least 10 nucleotides, and a second homology arm comprising a sequence of at least 20 nucleotides 3′ to said target sequence.

In some cases, the first homology arm comprises a sequence of at least 40, at least 50, at least 60, at least 70, at least 80, at least 90, at least 100, at least 110, at least 120, at least 130, at least 140, at least 150, at least 175, at least 200, at least 250, at least 300, at least 400, at least 500, at least 750, or at least 1000 nucleotides. In some cases, the second homology arm comprises a sequence of at least 40, at least 50, at least 60, at least 70, at least 80, at least 90, at least 100, at least 110, at least 120, at least 130, at least 140, at least 150, at least 175, at least 200, at least 250, at least 300, at least 400, at least 500, at least 750, or at least 1000 nucleotides.

In some cases, the first and second homology arms are homologous to a genomic sequence of a prokaryote. In some cases, the first and second homology arms are homologous to a genomic sequence of a bacteria. In some cases, the first and second homology arms are homologous to a genomic sequence of a fungus. In some cases, the first and second homology arms are homologous to a genomic sequence of a eukaryote.

In some cases, the engineered nuclease system further comprises a DNA repair template. The DNA repair template may comprise a double-stranded DNA segment. The double-stranded DNA segment may be flanked by one single-stranded DNA segment. The double-stranded DNA segment may be flanked by two single-stranded DNA segments. In some cases, the single-stranded DNA segments are conjugated to the 5′ ends of the double-stranded DNA segment. In some cases, the single stranded DNA segments are conjugated to the 3′ ends of the double-stranded DNA segment.

In some cases, the single-stranded DNA segments have a length from 1 to 15 nucleotide bases. In some cases, the single-stranded DNA segments have a length from 4 to 10 nucleotide bases. In some cases, the single-stranded DNA segments have a length of 4 nucleotide bases. In some cases, the single-stranded DNA segments have a length of 5 nucleotide bases. In some cases, the single-stranded DNA segments have a length of 6 nucleotide bases. In some cases, the single-stranded DNA segments have a length of 7 nucleotide bases. In some cases, the single-stranded DNA segments have a length of 8 nucleotide bases. In some cases, the single-stranded DNA segments have a length of 9 nucleotide bases. In some cases, the single-stranded DNA segments have a length of 10 nucleotide bases.

In some cases, the single-stranded DNA segments have a nucleotide sequence complementary to a sequence within the spacer sequence. In some cases, the double-stranded DNA sequence comprises a barcode, an open reading frame, an enhancer, a promoter, a protein-coding sequence, a miRNA coding sequence, an RNA coding sequence, or a transgene.

In some cases, the engineered nuclease system further comprises a source of Mg²⁺.

In some cases, the guide RNA comprises a hairpin comprising at least 8 base-paired ribonucleotides. In some cases, the guide RNA comprises a hairpin comprising at least 9 base-paired ribonucleotides. In some cases, the guide RNA comprises a hairpin comprising at least 10 base-paired ribonucleotides. In some cases, the guide RNA comprises a hairpin comprising at least 11 base-paired ribonucleotides. In some cases, the guide RNA comprises a hairpin comprising at least 12 base-paired ribonucleotides.

In some cases, the endonuclease comprises a sequence at least 70% identical to a variant of any one of SEQ ID NOs: 1, 6, 15, 30, 151, 292, or 319, or a variant thereof. In some cases, the endonuclease comprises a sequence at least 75% identical to a variant of any one of SEQ ID NOs: 1, 6, 15, 30, 151, 292, or 319, or a variant thereof. In some cases, the endonuclease comprises a sequence at least 80% identical to a variant of any one of SEQ ID NOs: 1, 6, 15, 30, 151, 292, or 319, or a variant thereof. In some cases, the endonuclease comprises a sequence at least 85% identical to a variant of any one of SEQ ID NOs: 1, 6, 15, 30, 151, 292, or 319, or a variant thereof. In some cases, the endonuclease comprises a sequence at least 90% identical to a variant of any one of SEQ ID NOs: 1, 6, 15, 30, 151, 292, or 319, or a variant thereof. In some cases, the endonuclease comprises a sequence at least 95% identical to a variant of any one of SEQ ID NOs: 1, 6, 15, 30, 151, 292, or 319, or a variant thereof.

In some cases, sequence may be determined by a BLASTP, CLUSTALW, MUSCLE, or MAFFT algorithm, or a CLUSTALW algorithm with the Smith-Waterman homology search algorithm parameters. The sequence identity may be determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In one aspect, the present disclosure provides an engineered guide RNA comprising a DNA-targeting segment. In some cases, the DNA-targeting segment comprises a nucleotide sequence that is complementary to a target sequence. In some cases, the target sequence is in a target DNA molecule. In some cases, the engineered guide RNA comprises a protein-binding segment. In some cases, the protein-binding segment comprises two complementary stretches of nucleotides. In some cases, the two complementary stretches of nucleotides hybridize to form a double-stranded RNA (dsRNA) duplex. In some cases, the two complementary stretches of nucleotides are covalently linked to one another with intervening nucleotides. In some cases, the engineered guide ribonucleic acid polynucleotide is capable of forming a complex with an endonuclease. In some cases, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-325, 420-431, 476-624, or 629. In some cases, the complex targets the target sequence of the target DNA molecule. In some cases, the DNA-targeting segment is positioned 3′ of both of the two complementary stretches of nucleotides.

In some cases, the double-stranded RNA (dsRNA) duplex comprises at least 8 ribonucleotides. In some cases, the double-stranded RNA (dsRNA) duplex comprises at least 9 ribonucleotides. In some cases, the double-stranded RNA (dsRNA) duplex comprises at least 10 ribonucleotides. In some cases, the double-stranded RNA (dsRNA) duplex comprises at least 11 ribonucleotides. In some cases, the double-stranded RNA (dsRNA) duplex comprises at least 12 ribonucleotides.

In some cases, the deoxyribonucleic acid polynucleotide encodes the engineered guide ribonucleic acid polynucleotide.

In one aspect, the present disclosure provides a nucleic acid comprising an engineered nucleic acid sequence. In some cases, the engineered nucleic acid sequence is optimized for expression in an organism. In some cases, the nucleic acid encodes an endonuclease. In some cases, the endonuclease is a Cas endonuclease. In some cases, the endonuclease is a class 2 endonuclease. In some cases, the endonuclease is a class2, type V Cas endonuclease. In some cases, the endonuclease is a class2, type V, novel subtype Cas endonuclease. In some cases, the endonuclease is derived from an uncultivated microorganism. In some cases, the organism is not the uncultivated organism.

In some cases, the endonuclease comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-325, 420-431, 476-624, or 629.

In some cases, the endonuclease may comprise a variant having one or more nuclear localization sequences (NLSs). The NLS may be proximal to the N- or C-terminus of the endonuclease. The NLS may be appended N-terminal or C-terminal to any one of SEQ ID NOs: 630-645, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 630-645.

In some cases, the organism is prokaryotic. In some cases, the organism is bacterial. In some cases, the organism is eukaryotic. In some cases, the organism is fungal. In some cases, the organism is a plant. In some cases, the organism is mammalian. In some cases, the organism is a rodent. In some cases, the organism is human.

In one aspect, the present disclosure provides an engineered vector. In some cases, the engineered vector comprises a nucleic acid sequence encoding an endonuclease. In some cases, the endonuclease is a Cas endonuclease. In some cases, the endonuclease is a class 2 Cas endonuclease. In some cases, the endonuclease is a class 2, type V Cas endonuclease. In some cases, the endonuclease is a class2, type V, novel subtype Cas endonuclease. In some cases, the endonuclease is derived from an uncultivated microorganism.

In some cases, the engineered vector comprises a nucleic acid described herein. In some cases, the nucleic acid described herein is a deoxyribonucleic acid polynucleotide described herein. In some cases, the vector is a plasmid, a minicircle, a CELiD, an adeno-associated virus (AAV) derived virion, or a lentivirus.

In one aspect, the present disclosure provides a cell comprising a vector described herein.

In one aspect, the present disclosure provides a method of manufacturing an endonuclease. In some cases, the method comprises cultivating the cell.

In one aspect, the present disclosure provides a method for binding, cleaving, marking, or modifying a double-stranded deoxyribonucleic acid polynucleotide. The method may comprise contacting the double-stranded deoxyribonucleic acid polynucleotide with an endonuclease. In some cases, the endonuclease is a Cas endonuclease. In some cases, the endonuclease is a class 2 Cas endonuclease. In some cases, the endonuclease is a class 2, type V Cas endonuclease. In some cases, the endonuclease is a class2, type V, novel subtype Cas endonuclease. In some cases, the endonuclease is in complex with an engineered guide RNA. In some cases, the engineered guide RNA is configured to bind to the endonuclease. In some cases, the engineered guide RNA is configured to bind to the double-stranded deoxyribonucleic acid polynucleotide. In some cases, the engineered guide RNA is configured to bind to the endonuclease and to the double-stranded deoxyribonucleic acid polynucleotide. In some cases, the double-stranded deoxyribonucleic acid polynucleotide comprises a protospacer adjacent motif (PAM).

In some cases, the double-stranded deoxyribonucleic acid polynucleotide comprises a first strand comprising a sequence complementary to a sequence of the engineered guide RNA and a second strand comprising the PAM. In some cases, the PAM is directly adjacent to the 5′ end of the sequence complementary to the sequence of the engineered guide RNA. In some cases, the endonuclease is not a Cpf1 endonuclease or a Cms1 endonuclease. In some cases, the endonuclease is derived from an uncultivated microorganism. In some cases, the double-stranded deoxyribonucleic acid polynucleotide is a eukaryotic, plant, fungal, mammalian, rodent, or human double-stranded deoxyribonucleic acid polynucleotide.

In one aspect, the present disclosure provides a method of modifying a target nucleic acid locus. The method may comprise delivering to the target nucleic acid locus the engineered nuclease system described herein. In some cases, the endonuclease is configured to form a complex with the engineered guide ribonucleic acid structure. In some cases, the complex is configured such that upon binding of the complex to the target nucleic acid locus, the complex modifies the target nucleic acid locus.

In some cases, modifying the target nucleic acid locus comprises binding, nicking, cleaving, or marking said target nucleic acid locus. In some cases, the target nucleic acid locus comprises deoxyribonucleic acid (DNA) or ribonucleic acid (RNA). In some cases, the target nucleic acid comprises genomic DNA, viral DNA, viral RNA, or bacterial DNA. In some cases, the target nucleic acid locus is in vitro. In some cases, the target nucleic acid locus is within a cell. In some cases, the cell is a prokaryotic cell, a bacterial cell, a eukaryotic cell, a fungal cell, a plant cell, an animal cell, a mammalian cell, a rodent cell, a primate cell, or a human cell.

In some cases, delivery of the engineered nuclease system to the target nucleic acid locus comprises delivering the nucleic acid described herein or the vector described herein. In some cases, delivery of engineered nuclease system to the target nucleic acid locus comprises delivering a nucleic acid comprising an open reading frame encoding the endonuclease. In some cases, the nucleic acid comprises a promoter. In some cases, the open reading frame encoding the endonuclease is operably linked to the promoter.

In some cases, delivery of the engineered nuclease system to the target nucleic acid locus comprises delivering a capped mRNA containing the open reading frame encoding the endonuclease. In some cases, delivery of the engineered nuclease system to the target nucleic acid locus comprises delivering a translated polypeptide. In some cases, delivery of the engineered nuclease system to the target nucleic acid locus comprises delivering a deoxyribonucleic acid (DNA) encoding the engineered guide RNA operably linked to a ribonucleic acid (RNA) pol III promoter.

In some cases, the endonuclease induces a single-stranded break or a double-stranded break at or proximal to the target locus. In some cases, the endonuclease induces a staggered single stranded break within or 3′ to said target locus.

In some cases, effector repeat motifs are used to inform guide design of MG nucleases. For example, the processed gRNA in Type V systems consists of the last 20-22 nucleotides of a CRISPR repeat. This sequence may be synthesized into a crRNA (along with a spacer) and tested in vitro, along with the synthesized nucleases, for cleavage on a library of possible targets. Using this method, the PAM may be determined. In some cases, Type V enzymes may use a “universal” gRNA. In some cases, Type V enzymes may need a unique gRNA.

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

### EXAMPLES

In accordance with IUPAC conventions, the following abbreviations are used throughout the examples:
A=adenine
C=cytosine
G=guanine
T=thymine
R=adenine or guanine
Y=cytosine or thymine
S=guanine or cytosine
W=adenine or thymine
K=guanine or thymine
M=adenine or cytosine
B=C, G, or T
D=A, G, or T
H=A, C, or T
V=A, C, or G

### Example 1—a Method of Metagenomic Analysis for New Proteins

Metagenomic samples were collected from sediment, soil, and animals. Deoxyribonucleic acid (DNA) was extracted with a Zymobiomics DNA mini-prep kit and sequenced on an Illumina HiSeq® 2500. Samples were collected with consent of property owners. Additional raw sequence data from public sources included animal microbiomes, sediment, soil, hot springs, hydrothermal vents, marine, peat bogs, permafrost, and sewage sequences. Metagenomic sequence data was searched using Hidden Markov Models generated based on known Cas protein sequences including class II type V Cas effector proteins to identify new Cas effectors. Novel effector proteins identified by the search were aligned to known proteins to identify potential active sites. This metagenomic workflow resulted in the delineation of the MG90, MG91A, MG91B, MG91C, MG118, MG119, MG120, MG122, and MG126 families described herein.

### Example 2—Discovery of MG90, MG91A, MG91B, MG91C, MG118, MG119, MG120, MG122, and MG126 Families of CRISPR Systems

Analysis of the data from the metagenomic analysis of Example 1 revealed new clusters of previously undescribed putative CRISPR systems comprising 9 families (MG90, MG91A, MG91B, MG91C, MG118, MG119, MG120, MG122, and MG126). The corresponding protein and nucleic acid sequences for these new enzymes and their exemplary subdomains are presented as SEQ ID NOs: 1-325, 420-431, 476-624, or 629.

### Example 3—Template DNA for Transcription and Translation

E. coli codon optimized sequences of all MG VU and CasPhi nucleases were ordered (Twist Biosciences) in a plasmid with a T7 promoter. Linear templates were amplified from the plasmids by PCR to include the T7 and nuclease sequence. Minimal array linear templates were amplified from sequences composed of a T7 promoter, native repeat, universal spacer, and native repeat, flanked by adapter sequences for amplification. The universal spacer matches the spacer in an 8N target library, where there are 8N mixed bases adjacent to the spacer for PAM determination. Three intergenic sequences near the ORF or CRISPR array were identified from the metagenomic contigs and ordered as gBlocks with flanking adapter sequences for amplification (Integrated DNA Technologies).

### Example 4—In vitro transcription of crRNA, Minimal Arrays, and sgRNA

RNA was produced by in vitro transcription using HiScribe™ T7 High Yield RNA Synthesis Kit and purified using the Monarch® RNA Cleanup Kit (New England Biolabs Inc.). Templates for T7 transcription varied. For crRNA, DNA oligos were designed with a T7 promoter, trimmed native repeat, and universal spacer. For minimal arrays the same templates as described above were used. For sgRNA, DNA ultramers were designed with a T7 promoter, trimmed tracrRNA, GAAA tetraloop, trimmed native repeat, and universal spacer Minimal array templates were amplified with adapter primers. The crRNA and sgRNA templates were ordered as reverse complements and annealed with a primer with the T7 promoter sequence in 1X IDT duplex buffer at 95° C. for two minutes followed by cooling to 22° C. at 0.1° C./second to produce a hybrid ds/ssDNA substrate suitable for transcription. After transcription, but prior to cleaning, each reaction was treated with DNAse I and incubated at 37° C. for 15 minutes. All transcription products were verified for yield and purity via RNA TapeStation or via a denaturing urea PAGE

### Example 5—TXTL Expression

Nucleases, intergenic sequences, and minimal arrays were expressed in transcription-translation reaction mixtures using myTXTL®Sigma 70 Master Mix Kit (Arbor Biosciences). The final reaction mixtures contained 5 nM nuclease DNA template. 12 nM intergenic DNA template, 15 nM minimal array DNA template, 0.1 nM pTXTL-P70a-T7map, and 1X of myXTL®Signa 70 Master Mix. The reactions were incubated at 29° C. for 16 hours then stored at 4° C.

### Example 6—PURExpress Expressions

10 nM of nuclease PC R templates were expressed at 37° C. for 3 hours with PURExpress® In Vitro Protein Synthesis Kit (New England Biolabs Inc.) for cleavage with in vitro transcribed RNA, These reactions were used to test in vitro cleavage with 50 nM sgRNA or minimal array RNA following the same procedure as described in the cleavage reactions section.

### Example 7—E. coli Expressions

Plasmids encoding the effector, intergenic sequence from the genomic contig, native repeat, and universal spacer sequences with a T7 promoter were transformed into B 21 DE3 or T7 Express lysY/Iq and cultured at 37° C. in 60 mL terrific broth media supplemented with 100 μg/mL of ampicillin. Expression was induced with 0.4 mM IPTG after cultures reached OD_{600 nm} of 0.5 and incubated at 16° C. overnight. 25 mL of cells were pelleted by centrifugation and resuspended in 1.5 mL of lysis buffer (20 mM Tris-HCl, 500 mM NaCl, 1 mM TCEP, 5% glycerol, 10 mM MgCl2 pH 7.5 with Pierce Protease Inhibitor, (Thermo Scientific™). Cells were then lysed by sonication, Supernatant and cell debris were separated by centrifugation.

### Example 8—Cleavage Reactions

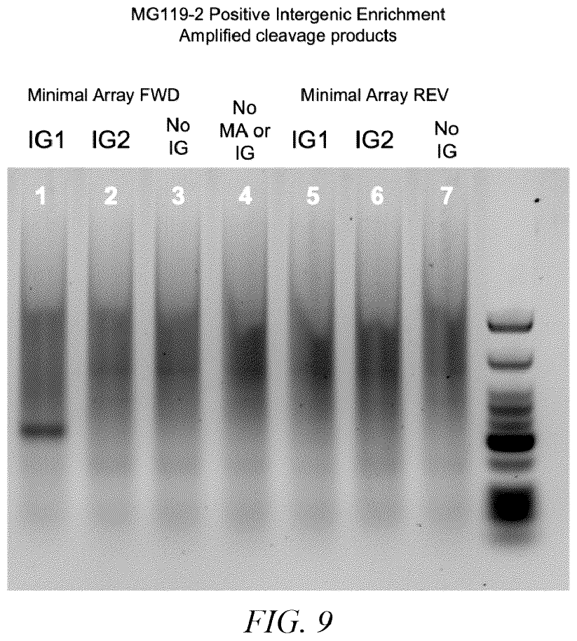

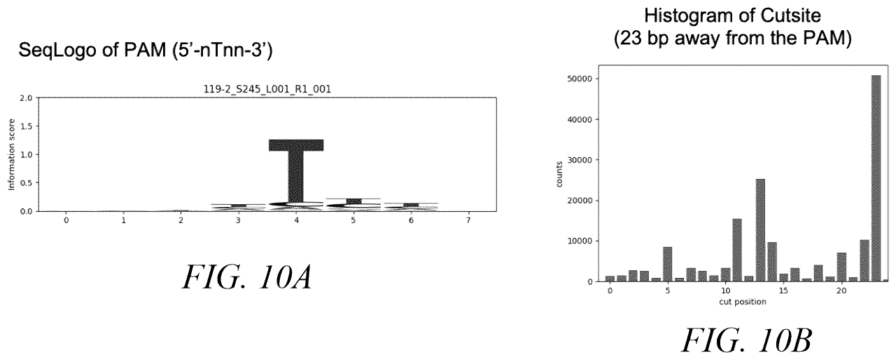

Plasmid library DNA cleavage reactions were carried out by mixing 5 nM of the target library, a 5-fold dilution of the TXTL or PURExpress expressions, 10 nM Tris-HC 10 nM MgCl₂, and 100 mM NaCl at 37° C. for 2 hours. For reactions with E. coli expressions, 10 μL of the clarified lysate was added. Reactions were stopped and cleaned with HighPrep™ PCR clean up beads (MAGBIO Genomics, Inc.) and eluted in Tris EDTA pH 8.0 buffer. 3 nM of the cleavage product ends were blunted with 3.33 μM dNTPs, 1X T4 DNA ligase buffer, and 0.167 U/μL of Klenow Fragment (New England Biolabs Inc.) at 25° C. for 15 minutes. 1.5 nM of the cleavage products were ligated with 150 nM adapters, 1X T4 DNA ligase buffer (New England Biolabs Inc.) 20 U/μL T4 DNA ligase (New England Biolabs Inc.) at room temperature for 20 minutes. The ligated products were amplified by PCR with NGS primers and sequenced by NGS to obtain the PAM. The in vitro activity of MG119-2 is depicted in FIG. 9, while the PAM determination for MG119-2 is depicted in FIG. 10.

### Example 9—RNAseq Library Prep of Intergenic Enrichment from TXTL and E. coli Lysates

RNA is extracted from TXTL and cell lysate expressions following the Quick-RNA™ Miniprep Kit (Zymo Research) and eluted in 30-50 μL of water. The total concentration of the transcripts were measured on a Nanodrop, Tapestation, and Qubit.

100 ng-1ug of total RNA from each sample were prepped for RNA sequencing using the NEBNext Small RNA Library Prep Set for Illumina (New England Biolabs Inc.). Amplicons between 150-300 bp were quantified by Tapestation and Qubit and pooled to a final concentration of 4 nM A final concentration of 12.5 pM was loaded into a MiSeq V3 kit and sequenced in a Miseq system (Illumina) for 176 total cycles. The RNAseq reads were used to identify the tracr sequence of the genes.

### Example 10—Predicted RNA Folding

Predicted RNA folding of the active single RNA sequence was computed at 37° C. using the method of Andronescu 2007. The shading of the bases corresponds to the probability of base pairing of that base.

### Example 11—In Vitro Cleavage Efficiency (Prophetic)

The protein is expressed in E. coli protease deficient B strain under T7 inducible promoter, the cells are lysed using sonication, and the His-tagged protein of interest is purified using HisTrap FF (GE Lifescience) Ni-NTA affinity chromatography on the AKTA Avant FPLC (GE Lifescience). Purity is determined using densitometry in ImageLab software (Bio-Rad) of the protein bands resolved on SDS-PAGE and InstantBlue Ultrafast (Sigma-Aldrich) coomassie stained acrylamide gels (Bio-Rad). The protein is desalted in a storage buffer composed of 50 mM Tris-HCl, 300 mM NaCl, 1 mM TCEP, 5% glycerol; pH 7.5 and stored at −80° C.

A target DNA is constructed that contains a spacer sequence and the PAM determined via NGS. In the case of degenerate bases in the PAM a single representative PAM is chosen for testing. The target DNA is 2200 bp of linear DNA derived from a plasmid via PCR amplification. The PAM and spacer are located 700 bp from one end. Successful cleavage results in fragments of 700 and 1500 bp.

The target DNA, in vitro transcribed single RNA, and purified recombinant protein are combined in cleavage buffer (10 mM Tris, 100 mM NaCl, 10 mM MgCl2) with an excess of protein and RNA and incubated for 5′ to 3 hours, usually 1 hr. The reaction is stopped via addition of RNAse A and incubation at 60°. The reaction is resolved on a 1.2% TAE agarose gel and the fraction of cleaved target DNA is quantified in ImageLab software.

### Example 12—Activity in E. coli (Prophetic)

For testing of nuclease activity in bacterial cells, strains are constructed with genome sequences containing the target spacer and corresponding PAM sequence specific to the enzyme of interest. Engineered strains are then transformed with the nuclease of interest and transformants are then subsequently made chemocompetent and transformed with 50 ng of single guides either specific to the target sequence, on target, or non specific to the target, off target. After heat shock, transformations are recovered in SOC for 2 hrs at 37° C., and nuclease efficiency is determined by a 5-fold dilution series grown on induction media. Colonies are quantified from the dilution series in triplicate.

### Example 13—Activity in Mammalian Cells (Prophetic)

To show targeting and cleavage activity in mammalian cells, the protein sequences are cloned into 2 mammalian expression vectors, one with a C-terminal SV40 NLS and a 2A-GFP tag and one with no GFP tag and 2 NLS sequences, one on the N-terminus and one on the C-terminus. Alternative NLS sequences that can also be used. The DNA sequence for the protein can be the native sequence, the E. coli codon optimized sequence, or the mammalian codon optimized sequence. The single guide RNA sequence with a gene target of interest is also cloned into a mammalian expression vector. The two plasmids are cotransfected into HEK293T cells. 72 hr after co-transfection of the expression plasmid and a sgRNA targeting plasmid into HEK293T cells, the DNA is extracted and used for the preparation of an NGS-library. Percent NHEJ is measured via indels in the sequencing of the target site to demonstrate the targeting efficiency of the enzyme in mammalian cells. At least 10 different target sites are chosen for testing each protein's activity.

### Example 14—Characterization of Compact Type V Nucleases in the MG119 Family

### In Silico Identification of Novel Compact Type V Nucleases in the MG119 Family

Discovery of predicted proteins related to nuclease sequences in the MG119 family of compact type V nucleases was based on homology searches. Searches were performed using HMMER software (http://hmmer.org/). Type V nuclease sequence hits were retained if they met the following criteria: (i) the hmmsearch e-value was ≤10⁻⁵′, (ii) the genes encoding the nuclease were within 1 kb from a CRISPR array, and (iii) the amino acid sequence length ranged between 350 and 700 aa. MMSeqs2 (https://github.com/soedinglab/MMseqs2) was used to cluster sequences at 100% amino acid identity, with coverage mode 1 and 80% coverage of the target sequence (parameters—cov-mode 1—c 0.8—min-seq-id 1.0). Sequence representatives were chosen to build a multiple sequence alignment using MAFFT (https://mafft.cbrc.jp/alignment/software/) with the Needleman-Wunsch algorithm for global alignment, and FastTree (https://doi.org/10.1371/journal.pone.0009490) was used to build a phylogenetic tree. Careful examination of individual clades on the phylogenetic tree, including the nuclease gene's genomic context, led to the identification of several novel compact type V nuclease sequences in the MG119 family (SEQ ID NOs: 476-624 and 629).

In Vitro Characterization to Identify Putative tracrRNAs

To identify putative tracrRNA sequences, e.g., for nuclease MG119-2, adjacent intergenic sequences and a minimal array were expressed in transcription-translation reaction mixtures using myTXTL®Sigma 70 Master Mix Kit (Arbor Biosciences). The final reaction mixtures contained 5 nM nuclease DNA template, 12 nM intergenic DNA template, 15 nM minimal array DNA template, 0.1 nM pTXTL-P70a-T7map, and 1X of myTXTL®Sigma 70 Master Mix. The reactions were incubated at 29° C. for 16 hours, then stored at 4° C.

Ribonucleoprotein complexes were tested via in vitro cleavage reactions. Plasmid DNA library cleavage reactions were carried out by mixing 5 nM of the target plasmid DNA library representing all possible 8N PAMs, a 5-fold dilution of the TXTL expressions, 10 nM Tris-HCl, 10 nM MgCl₂ and, 100 mM NaCl at 37° C. for 2 hours. Reactions were stopped and cleaned with HighPrep™ PCR clean up beads (MAGBIO Genomics, Inc.) and eluted in Tris EDTA pH 8.0 buffer.

To obtain the PAM sequences, 3 nM of the cleavage product ends were blunted with 3.33 pM dNTPs, 1X T4 DNA ligase buffer, and 0.167 U/μL of Klenow Fragment (New England Biolabs Inc.) at 25° C. for 15 minutes. 1.5 nM of the cleavage products were ligated with 150 nM adapters, 1X T4 DNA ligase buffer (New England Biolabs Inc.), and 20 U/μL T4 DNA ligase (New England Biolabs Inc.) at room temperature for 20 minutes. The ligated products were amplified by PCR with NGS primers and sequenced by NGS.

To obtain the sequence of the tracrRNA and the crRNA, RNA was extracted from TXTL lysate following the Quick-RNA™ Miniprep Kit (Zymo Research) and eluted in 30-50 μL of water. 100 ng-1 pg of total RNA from each sample were prepped for RNA sequencing using the NEBNext Small RNA Library Prep Set for Illumina (New England Biolabs Inc.). Amplicons between 150-300 bp were quantified by Tapestation and Qubit and pooled to a final concentration of 4 nM. A final concentration of 12.5 pM was loaded into a MiSeq V3 kit and sequenced in a Miseq system (Illumina) for 176 total cycles. The RNAseq reads were used to identify the tracr sequence of the genes by mapping back to the original sequences.

In Silico Search for Novel tracrRNA Sequences

To identify additional non-coding regions containing potential tracrRNAs, the sequence of the active tracrRNA was mapped to other contigs containing nucleases in the same nuclease family (e.g. MG119-1 and MG119-3). The newly identified sequences were used to generate covariance models to predict additional tracrRNAs. Covariance models were built from a multiple sequence alignment (MSA) of the active and predicted tracrRNA sequences. The secondary structure of the MSA was obtained with RNAalifold (Vienna Package), and the covariance models were built with Infernal packages (http://eddylab.org/infemal/). Other contigs containing candidate nucleases were searched using the covariance models with the Infernal command ‘cmsearch’. TracrRNA candidates were tested in vitro (see below), and in an iterative process, sequences from active candidates were used to improve the covariance models and search for additional tracrRNAs in the intergenic regions associated with other nuclease candidates.

sgRNA Design

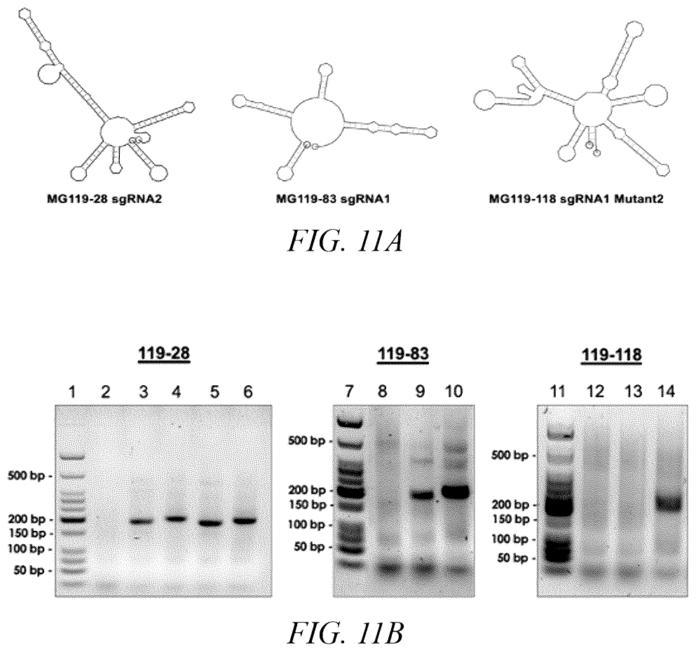

Predicted tracrRNAs obtained from the covariance models and their associated CRISPR repeat sequence were modified to generate sgRNAs (FIG. 11A) as follows: the 3′ end of the predicted tracrRNA sequence as well as the 5′ end of the repeat sequence were trimmed, and then connected with a GAAA tetraloop.

### In Vitro Cleavage Reactions to Confirm Nuclease Activity and Enable PAM Determination

5 nM of nuclease amplified DNA templates and 25 nM sgRNA amplified DNA templates (including one of the spacer sequences listed in Table 2) were expressed at 37° C. for 3 hours with PURExpress® In Vitro Protein Synthesis Kit (New England Biolabs Inc.). Plasmid library DNA cleavage reactions were carried out by mixing 5 nM of the target library representing all possible 8N PAMs, a 5-fold dilution of PURExpress expressions, 10 mM Tris-HCl pH 7.9, 10 mM MgCl₂, 100 μg/mL BSA, and 50 mM NaCl (NEB 2.1 Buffer, NEB Inc.) at 37° C. for 2 hours. Reactions were stopped and cleaned with HighPrep™ PCR clean up beads (MAGBIO Genomics, Inc.) and eluted in Tris EDTA pH 8.0 buffer. 3 nM of the cleavage product ends were blunted with 3.33 μM dNTPs, 1X T4 DNA ligase buffer, and 0.167 U/μL of Klenow Fragment (New England Biolabs Inc.) at 25° C. for 15 minutes. 1.5 nM of the cleavage products were ligated with 150 nM adapters, 1X T4 DNA ligase buffer (New England Biolabs Inc.), and 20 U/μL T4 DNA ligase (New England Biolabs Inc.) at room temperature for 20 minutes. The ligated products were amplified by PCR with NGS primers and sequenced by NGS to obtain the PAM. Active proteins that successfully cleaved the PAM library yielded a band around 188 or 205 bp in an agarose gel, depending on which target site was encoded in the sgRNA (FIG. 11B).

TABLE 2
Spacer sequences for tested guides

| Code | Sequence |
|---|---|
| U67 spacer | GTCGAGGCTTGCGACGTGGT |
| U40 spacer | TGGAGATATCTTGAACCTTG |

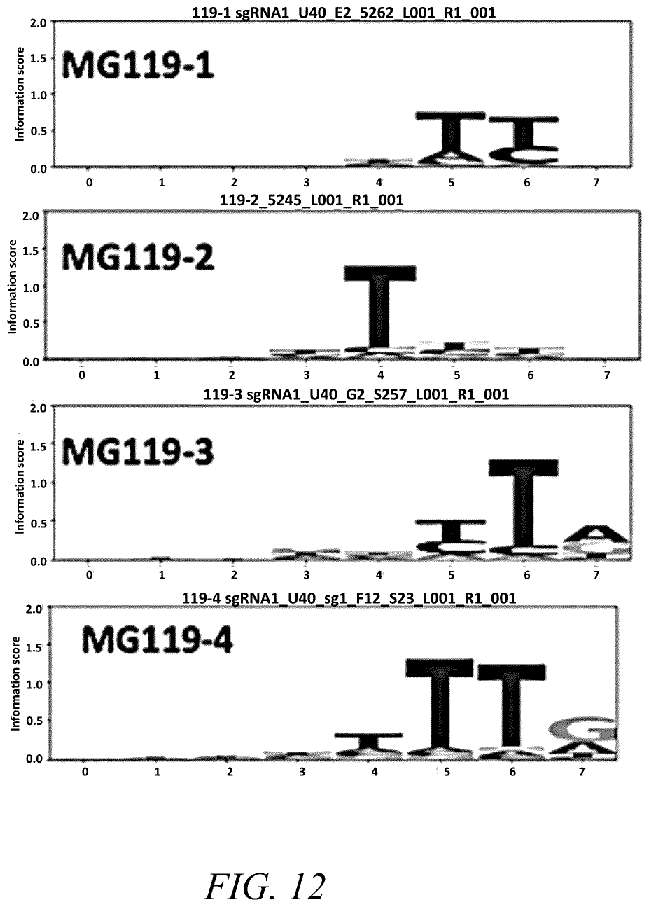

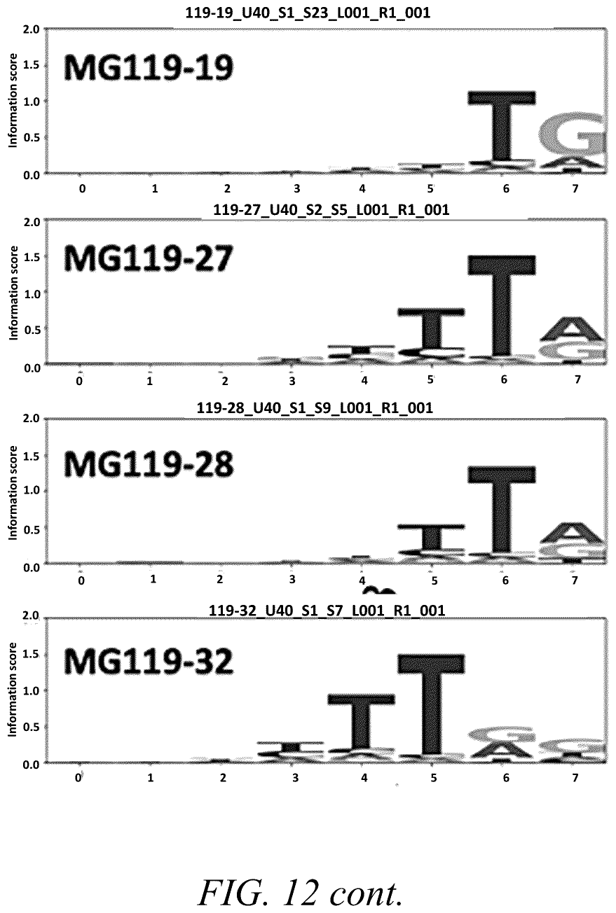

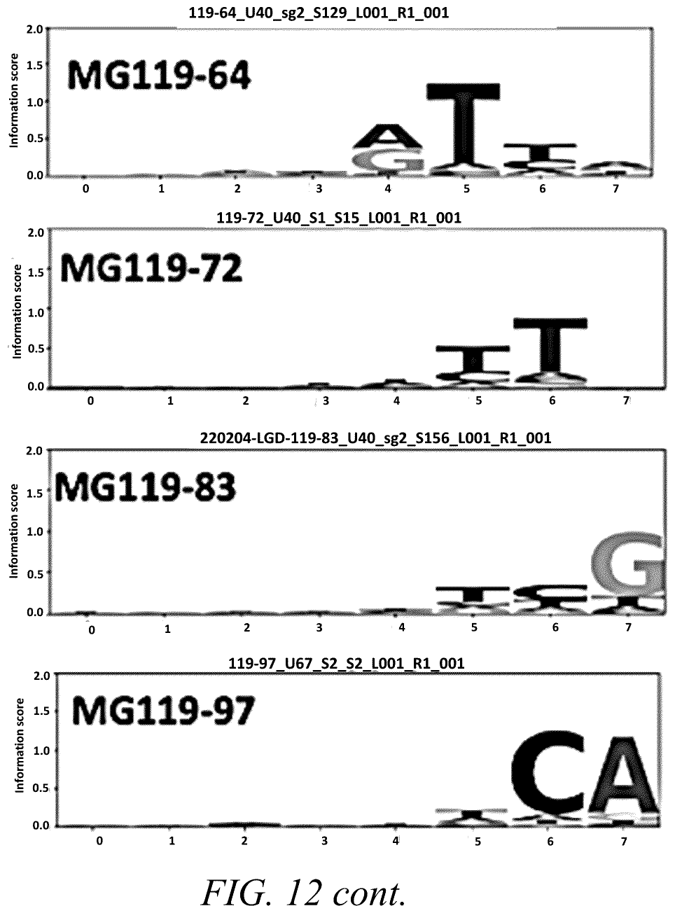

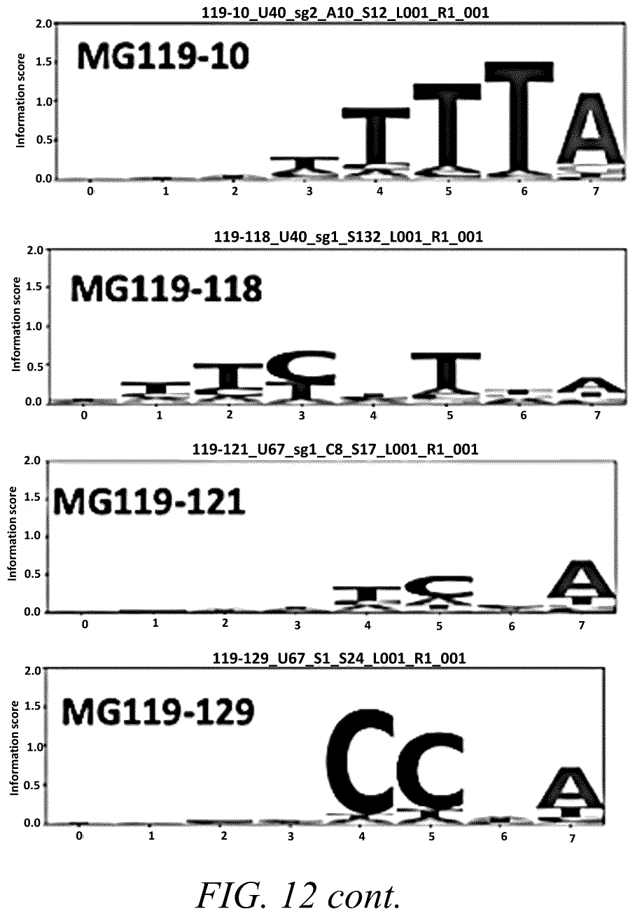

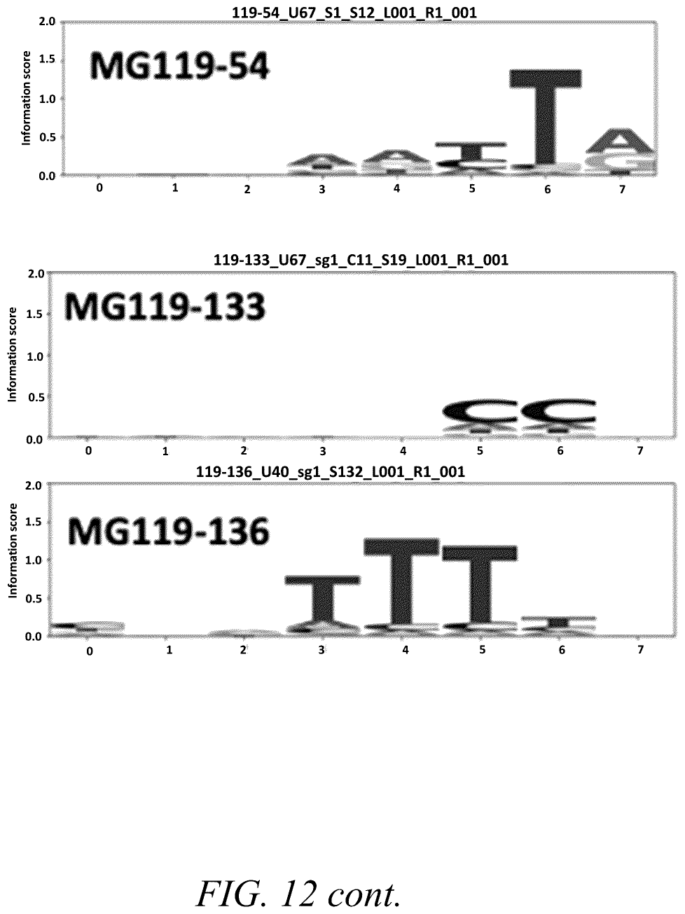

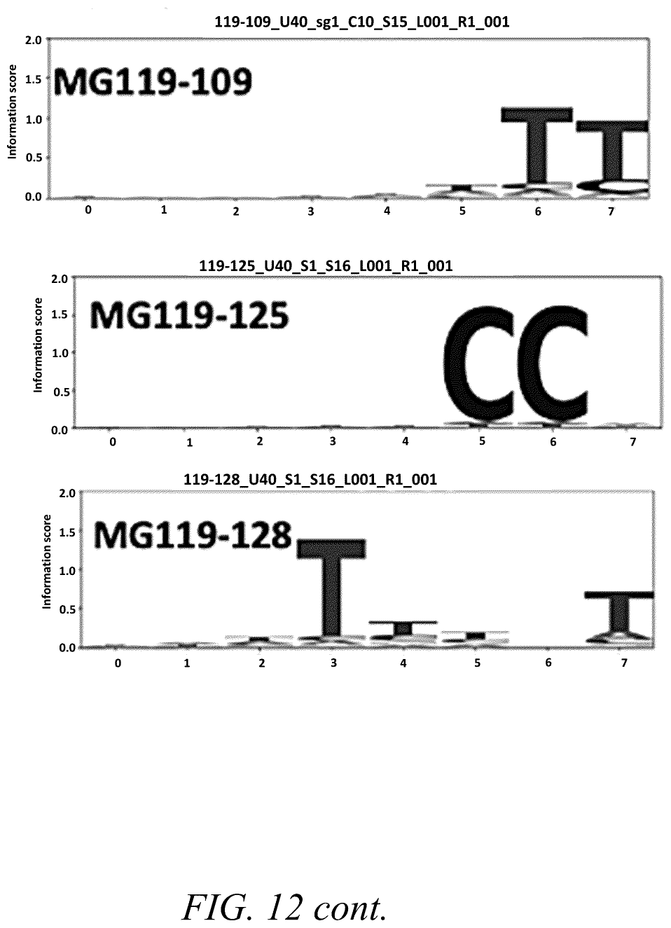

The PAMs recognized by MGT19 nucleases are shown as sequence logos made with Seqlog maker (FIG. 12). The preferred cut position on target strand of the protospacer sequence complementary to the U40 spacer is listed in Table 3.

TABLE 3
MG119 nucleases preferred cutsites in the protospacer sequence

| Nuclease | sgRNA | Cutsite |
|---|---|---|
| 119-1 | MG119-1_sgRNA1 | 20 & 23 |
| 119-2 | MG119-2_sgRNA1_Mutant1 | 22 |
| 119-3 | MG119-3_sgRNA1_Mutant1 | 22-23 |
| 119-4 | MG119-4_sgRNA1 | 22-23 |
| 119-10 | MG119-10_sgRNA1 | 22-23 |
| 119-19 | MG119-19_sgRNA1 | 23 |
| 119-27 | MG119-27_sgRNA2_Mutant2 | 22-23 |
| 119-28 | MG119-28_sgRNA2 | 22-23 |
| 119-32 | MG119-32_sgRNA1 | 23 |
| 119-54 | MG119-54_sgRNA1 | 22 |
| 119-64 | MG119-64_sgRNA2 | 20 |
| 119-72 | MG119-72_sgRNA1 | 23 |
| 119-83 | MG119-83_sgRNA1 | 23 |
| 119-97 | MG119-97_sgRNA1_Mutant1 | 22 |
| 119-109 | MG119-109_sgRNA1 | 24-25 |
| 119-118 | MG119-118_sgRNA1_Mutant2 | 23 |
| 119-121 | MG119-121_sgRNA1_Mutant1 | 20 & 22 |
| 119-125 | MG119-125_sgRNA1 | 22-23 |
| 119-128 | MG119-128_sgRNA2_Mutant1 | 22 |
| 119-129 | MG119-129_sgRNA1_Mutant1 | 22-23 |
| 119-133 | MG119-133_sgRNA1_Mutant1 | 22 |
| 119-136 | MG119-136_sgRNA1_Mutant2 | 23 |
| 119-137 | MG119-137_sgRNA1 | 22-23 |

### Protein Expression and Purification

Isolating pure and functional proteins is essential for extensive in vitro analysis of biochemical properties and mechanistic studies. The expression and purification of MG119 candidates was optimized to obtain proteins of sufficient quantity and quality for such characterizations. All constructs were expressed in K col (NEBExpress I^{q} Competent E. coli, NEB C3037I). Constructs were expressed in either the pMGB expression vector (MBP-fused), the pMGBΔ expression vector (no fusion protein), or both.

### Protein Expression

Protein expression protocols for pMGB and pMGBΔ constructs are identical. Cultures were grown at 37° C. in 2×YT media (1.6% tryptone, 1% yeast extract, 0.5% NaCl) or TB media (Teknova T0690) with 100 μg/L Carbenicillin. At OD600≈0.8−1.2, cultures were induced with 0.5 mM IPTG (GoldBio 12481) and incubated at 18° C. overnight or 24° C. for 4-6 hrs, depending on construct. Cultures were then harvested by centrifugation at 6,000×g for 10 min, and pellets were resuspended in Nickel_A Buffer (50 mM Tris pH 7.5, 750 mM NaCl, 10 mM MgCl₂, 20 mM imidazole, 0.5 mM EDTA, 5% glycerol, 0.5 mM TCEP)+protease inhibitors (Pierce Protease Inhibitor Tablets, EDTA-free, ThermoFisher A32965) and stored at −80° C.

Protein Purification—pMGBΔ Expression Vector

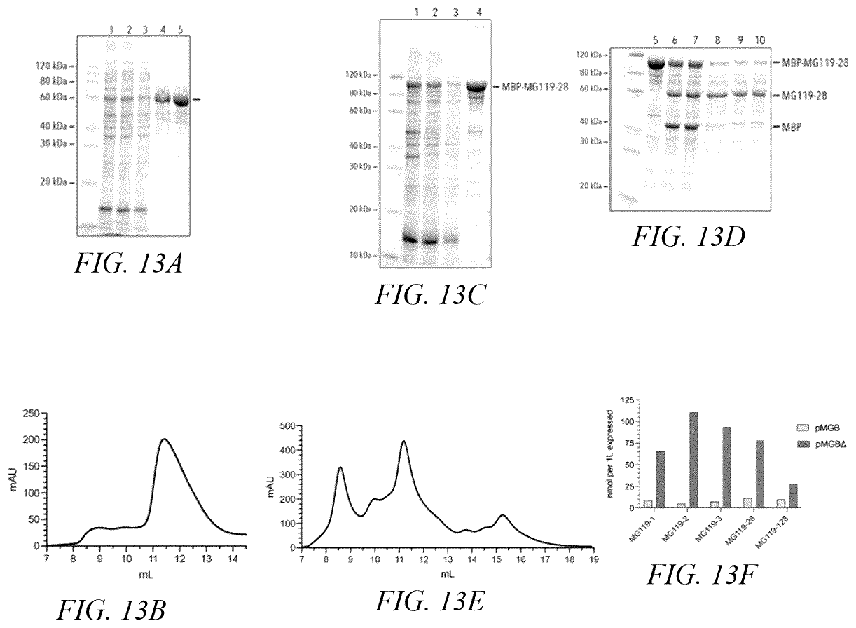

Proteins expressed in this vector have the following sequence architecture: 6×His-(GS)2-PSP-nucleoplasmin bipartite NLS-(GGS)1-(GS)1-MG119-X-(GGS)3-SV40 NLS (Table 5). Proteins expressed in this vector are denoted MG119-XΔ. Cell pellets were thawed and the volume supplemented to 120 mL with Cf=0.5% n-Octyl-β-D-glucoside detergent (P212121, CI-00234). Samples were sonicated in an ice-water bath at 75% amplitude for a total processing time of 3 min using a 15 s on/45 s off cycle. Lysates were clarified by centrifugation at 30,000×g for 25 min, and supernatants batch bound to 5 mL Ni-NTA resin (HisPur Ni-NTA Resin, ThermoFisher 88223) for ≥20 min. Samples were loaded onto a gravity column and washed with 30 CV Nickel_A Buffer, then eluted in 4 CV Nickel_B Buffer (Nickel_A Buffer+250 mM imidazole) before concentrating in a 50 kDa MWCO concentrator (Amicon Ultra-15, MilliporeSigma UFC9050). Samples were taken throughout the purification process and run on an SDS-PAGE protein gel (BioRad #4568126), which was imaged on a ChemiDoc in the stain-free channel following 5 min UV activation (FIG. 13A). ΔMBP constructs were then loaded onto an S200i 10/300 GL column (Cytiva 28-9909-44) and run into Nickel_A buffer (FIG. 13B). Peak fractions were pooled and concentrated in a 50 kDa MWCO concentrator. Purification of proteins expressed in the pMGBΔ vector typically yielded 25-125 nmol protein per L expression culture (FIG. 13F).

Protein Purification—pMGB Expression Vector

Proteins expressed in this vector have the following sequence architecture: 6×His-(GS)1-MBP-(GS)1-TEV-nucleoplasmin bipartite NLS-(GGGGS)3-(GS)1-MG119-X-(GGS)3-SV40 NLS (Table 5). MBP-fused constructs were purified identically to pMGBΔ proteins through lysis, clarification, affinity purification, and elution in Nickel_B (FIG. 13C). Following protein concentration in a 50 kDa MWCO concentrator, TEV protease (GenScript Z03030) was added to each sample (Cf=1 UI/μL) and incubated at 4° C. overnight, gently rotating end-over-end. Samples were centrifuged (21,000×g, 4° C., 10 min) to pellet aggregates, and the supernatant was then batch-bound to 3 mL amylose resin (NEB E8021L) for 30 min at 4° C., then loaded onto a gravity column. The flow-through was collected and concentrated in a 50 kDa MWCO concentrator (FIG. 13D). Again, samples were centrifuged (21,000×g, 4° C., 10 min) to pellet aggregates before loading on an S200i 10/300 GL column and run into Nickel_A buffer (FIG. 13E). Peak fractions were pooled and concentrated in a 50 kDa MWCO concentrator. Samples were taken throughout the purification process and run on an SDS-PAGE protein gel (BioRad #4568126), which was imaged on a ChemiDoc in the stain-free channel following 5 min UV activation (FIG. 13D).

A select few MG119 candidates were purified both from the pMGB and pMGBΔ expression vectors. A comparison of final protein yield, normalized to the initial expression culture volume, shows a trend of higher expression yields from the pMGBΔ vector (FIG. 13E). Purification of proteins expressed in the pMGBΔ vector typically yielded 2-15 nmol protein per L expression culture (FIG. 13E). Protein purification yields are shown in Table 4.

TABLE 4
Protein purification yields

| | Expression | Expression | Yield |
|---|---|---|---|
| Nuclease | Vector | Media | (nmol) |
| MG119-1 | pMGB | TB | 8.8 |
| MG119-1 | pMGBΔ | TB | 105.2 |
| MG119-2 | pMGB | 2xYT | 5.0 |
| MG119-2 | pMGBΔ | 2xYT | 95.0 |
| MG119-3 | pMGB | 2xYT | 7.6 |
| MG119-3 | pMGBΔ | 2xYT | 78.1 |
| MG119-27 | pMGB | 2xYT | 11.9 |
| MG119-28 | pMGB | 2xYT | 11.6 |
| MG119-28 | pMGBΔ | 2xYT | 102.2 |
| MG119-32 | pMGB | 2xYT | 4.3 |
| MG119-54 | pMGB | 2xYT | 5.6 |
| MG119-64 | pMGB | 2xYT | 2.1 |
| MG119-97 | pMGB | 2xYT | 4.3 |
| MG119-109 | pMGB | 2xYT | 4.1 |
| MG119-121 | pMGB | 2xYT | 8.2 |
| MG119-128 | pMGB | 2xYT | 9.9 |
| MG119-128 | pMGBΔ | 2xYT | 37.0 |
| MG119-129 | pMGB | 2xYT | 2.8 |
| MG119-136 | pMGB | 2xYT | 14.3 |
| MG119-137 | pMGB | 2xYT | 10.4 |

TABLE 5
Sequence element glossary

| | Element amino |
|---|---|
| Element name | acid sequence |
| 6xHis | HHHHHH |
| (GS)ₙ | GS |
| (GGS)ₙ | GGS |
| (GGGGS)ₙ | GGGGS |
| PSP | LEVQFQGP |
| TEV | ENLYFQG |
| Nucleoplasmin bipartite NLS | KRPAATKKAGQAKKKK |
| SV40 NLS | PKKKRKV |

In Vitro Cleavage Efficiency with Purified Protein

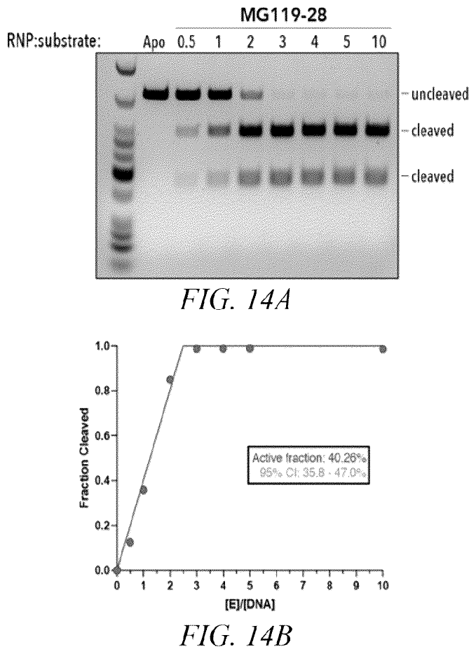

The active fraction of protein aliquots was determined in a linear DNA substrate cleavage assay. Effector proteins were preincubated with a 2-fold molar excess of sgRNA for 20 min at room temperature to form the ribonucleoprotein complex (RNP). Reactions were set up using 25 nM DNA substrate and a titration of RNP from 0.25X to 10X molar excess over substrate. The reaction buffer composition was 10 mM Tris pH 7.5, 10 mM MgCl₂, and 100 mM NaCl. The DNA substrate is 522 bp long. Successful cleavage results in fragments of 172 and 350 bp. The reaction was incubated at 37° C. for 60 min, then incubated at 75° C. for 10 min. RNase (NEB T3018) was added to each reaction (Cf=0.33 μg/μL), and samples were incubated at 37° C. for 10 min. Proteinase K (NEB P8107) was added to each reaction (Cf=60 units/mL), and samples were incubated at 55° C. for 15 min. The entirety of each reaction was then run on a 1.5% agarose gel with GelGreen dye (Biotium, #41005) (FIG. 14A) and imaged on a ChemiDoc in the GelGreen channel. Percent cleaved substrate was calculated for each lane through densitometry analysis using BioRad's Image Lab software (Version 6.1.0 build 7). Active fraction was determined by the slope of the linear range of cleavage (FIG. 14B).

In Vitro Cleavage of Purified Hepa1-6 Genomic DNA with Purified Protein

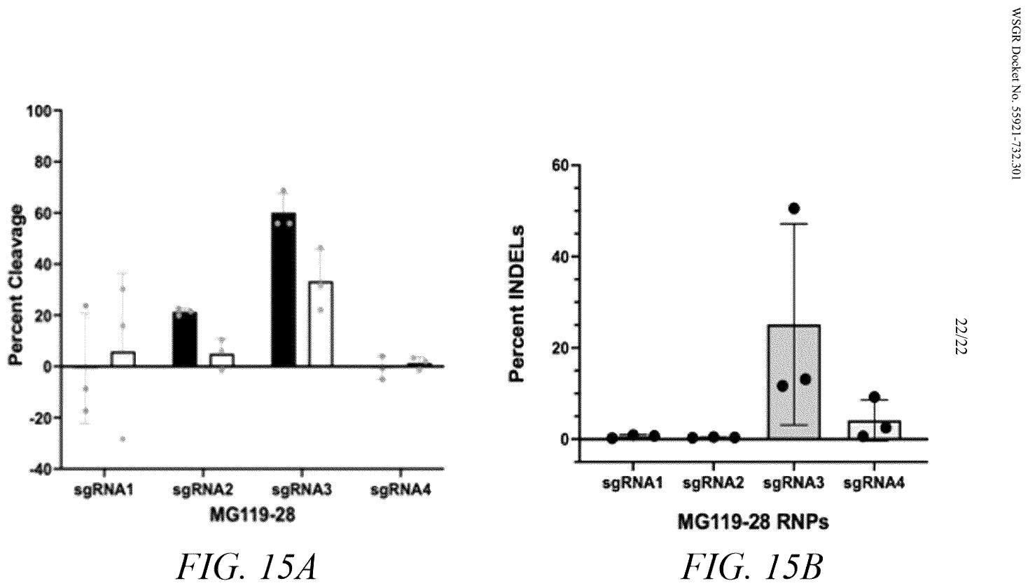

To assess cleavage of purified mouse Hepa1-6 genomic DNA (gDNA), the mouse albumin gene was targeted at intron 1 (Table 6). gDNA was extracted from Hepa1-6 cell pellets with 8 million cells following the Purelink™ Genomic DNA Mini kit (Invitrogen) and eluted in 10 mM TrisHCl at pH 8. sgRNAs were ordered from Integrated DNA technologies (IDT) at 2 nmol then resuspended in 10 mM Tris EDTA Buffer at 20 μM (Table 6). Ribonucleoproteins (RNPs) were made by pre-incubating nucleases with targeting or non-targeting guides at a 1:2 molar ratio for 30 minutes at room temperature in 1X effector buffer (100 mM NaCl, 10 mM MgCl₂, 10 mM Tris HCl, at pH 7.5). All reactions were done in replicates of three including negative controls with no sgRNA. Following RNP formation, RNP was added to a digest reaction containing 20 ng/μL of the purified gDNA in 1X effector buffer and incubated at 37° C. for 1 hour. The nuclease was tested at two final concentrations, 7.8 and 15.6 nM. These concentrations were normalized by dividing the targeted concentrations with the active fractions for each nuclease. Following incubation, these reactions were immediately moved to 4° C., diluted 30X in water, then prepared for qPCR in a mastermix containing 1X PrimeTime® Gene Expression Master Mix, 10 μM forward primer, 10 μM reverse primer, and 5 μM 5′-FAM and ZEN/Iowa Black fluorescence quencher Taqman probe (IDT) (Table 7). The AriaMx Real-Time PCR System (Agilent) was used with the following cycles 1) at 95° C. for 15 minutes, 2) at 95° C. for 5 seconds, and 3) at 60° C. for 1 min, where steps 2-3 were repeated 40X. Cq values were used to calculate the gDNA percent cleavage of each reaction following the Percent Cleavage Equation (below). All were normalized to the non-targeting control reactions. FIG. 15A illustrates an example of an average 60% gDNA cleavage by MG119-28 and sgRNA3 and 21% cleavage with sgRNA2 at the higher concentration of protein used.

$$Percent Cleavage Equation \% Cleavage = 100 - ( {2}^{- ( Cq ( experimental ) - Cq ( non targeting control ) )} \times 100 )$$

TABLE 6
Targeting sequences in mouse albumin intron 1
and chemically modified sgRNAs (IDT)

| sgRNA Name | Sequence (5′-3′) | Mouse Albumin Target (5′-3′) |
|---|---|---|
| 119-28 | mU*mU*mG*rArArArUrArAr | AAGATTGATGAAGACAACT |
| sgRNA1_Mouse_Alb | ArArUrGrArArUrUrUrCrArAr | A |
| | ArCrCrCrCrUrUrCrGrGrGrGrG | |
| | rArGrGrGrCrGrCrGrUrUrGrGr | |
| | ArGrCrGrCrCrUrUrArGrUrUrU | |
| | rGrArGrGrUrGrCrArGrArArUr | |
| | CrArArArArArArArCrUrGrCrG | |
| | rArCrGrArUrGrGrArGrGrUrCr | |
| | GrUrUrUrCrArGrUrCrUrCrUrG | |
| | rUrArCrArCrUrCrArArArArAr | |
| | ArUrUrCrArCrUrUrGrArGrAr | |
| | ArArUrCrArArGrUrGrArArUr | |
| | ArUrCrCrArArCrArArGrArUrU | |
| | rGrArUrGrArArGrArCrArA*m | |
| | C*mU*mA | |
| 119-28 | mU*mU*mG*rArArArUrArAr | GGTCAGTGAAGAGAAGAA |
| sgRNA2_Mouse_Alb | ArArUrGrArArUrUrUrCrArAr | CA |
| | ArCrCrCrCrUrUrCrGrGrGrGrG | |
| | rArGrGrGrCrGrCrGrUrUrGrGr | |
| | ArGrCrGrCrCrUrUrArGrUrUrU | |
| | rGrArGrGrUrGrCrArGrArArUr | |
| | CrArArArArArArArCrUrGrCrG | |
| | rArCrGrArUrGrGrArGrGrUrCr | |
| | GrUrUrUrCrArGrUrCrUrCrUrG | |
| | rUrArCrArCrUrCrArArArArAr | |
| | ArUrUrCrArCrUrUrGrArGrAr | |
| | ArArUrCrArArGrUrGrArArUr | |
| | ArUrCrCrArArCrGrGrUrCrArG | |
| | rUrGrArArGrArGrArArGrA*m | |
| | A*mC*mA | |
| 119-28 | mU*mU*mG*rArArArUrArAr | AGTGTAGCAGAGAGGAACC |
| sgRNA3_Mouse_Alb | ArArUrGrArArUrUrUrCrArAr | A |
| | ArCrCrCrCrUrUrCrGrGrGrGrG | |
| | rArGrGrGrCrGrCrGrUrUrGrGr | |
| | ArGrCrGrCrCrUrUrArGrUrUrU | |
| | rGrArGrGrUrGrCrArGrArArUr | |
| | CrArArArArArArArCrUrGrCrG | |
| | rArCrGrArUrGrGrArGrGrUrCr | |
| | GrUrUrUrCrArGrUrCrUrCrUrG | |
| | rUrArCrArCrUrCrArArArArAr | |
| | ArUrUrCrArCrUrUrGrArGrAr | |
| | ArArUrCrArArGrUrGrArArUr | |
| | ArUrCrCrArArCrArGrUrGrUrA | |
| | rGrCrArGrArGrArGrGrArA*m | |
| | C*mC*mA | |
| 119-28 | mU*mU*mG*rArArArUrArAr | TCTGTGGAAACAGGGAGAG |
| sgRNA4_Mouse_Alb | ArArUrGrArArUrUrUrCrArAr | A |
| | ArCrCrCrCrUrUrCrGrGrGrGrG | |
| | rArGrGrGrCrGrCrGrUrUrGrGr | |
| | ArGrCrGrCrCrUrUrArGrUrUrU | |
| | rGrArGrGrUrGrCrArGrArArUr | |
| | CrArArArArArArArCrUrGrCrG | |
| | rArCrGrArUrGrGrArGrGrUrCr | |
| | GrUrUrUrCrArGrUrCrUrCrUrG | |
| | rUrArCrArCrUrCrArArArArAr | |
| | ArUrUrCrArCrUrUrGrArGrAr | |
| | ArArUrCrArArGrUrGrArArUr | |
| | ArUrCrCrArArCrUrCrUrGrUrG | |
| | rGrArArArCrArGrGrGrArG*m | |
| | A*mG*mA | |

TABLE 7
DNA oligos used for qPCR

| Oligo Names | Oligo sequences |
|---|---|
| 611F_HE | TGCACAGATATAAACACTTAACGGG |
| 869R_HE | GGGCGATCTCACTCTTGTCT |
| 680_HE Taqman Probe | 5′-FAM-AGCAGAGAGGAACCATTGCCACCT |
| | TCAG |

In Vivo Cleavage of Genomic DNA in Hepa 1-6 Cells with Purified Protein

In cell editing was demonstrated with RNP complexes of nucleases and guides targeting the mouse albumin gene at intron 1 (Table 6). Hepa1-6 cells were thawed, washed, and resuspended in Dulbecco's Modified Eagle Medium (DMEM, 1000 FBS, and 1% Pen-strep). Cells were seeded at a density of 4×10⁶ cells per 15 cm dish in 30 mL of media at 37° C. After two days when the cells reached 70-80% confluency, the cells were split. Cells were trypsinized with 0.25% trypsin, then incubated at 37° C. for 30 seconds. DMEM was added then split into 3 mL and further diluted with 27 mL of media. Split cells were incubated for another two days. Prior to nucleofection, the media was aspirated from the plates, and cells were washed with 1X phosphate buffer saline (PBS, Gibco™) pH 7.2 before trypsinizing. Trypsin was neutralized and cells were resuspended with DMEM. Cells in the cell suspension were counted with a Countess 3 FL (Invitrogen) to calculate the volume of cells to pellet. Each treatment downstream required a total of 100,000 cells. Cells were centrifuged at 300×g for 7 minutes in a sorvall X Pro Series Centrifuge (Thermo Fisher), then washed in PBS pH 7.2 before resuspending in Nucleofector™ Solution from the Amaxa™ 4D-Nucleofector™ Kit (Lonza).

RNP complexes were individually prepared by incubating 120 pmol of the nucleases with 120 pmol of the guides for 90 min at room temperature. 20 μL of the prepared cells were added to the RNPs. Nucleofections were done as recommended by the Amaxa™ 4D-Nucleofector™ Protocol in a 4D-Nucleofector™ System (Lonza). The nucleofected cells were transferred from the nucleofection cassettes to the 24 well plates, each well containing 500 μL of media. Following a two day incubation, gDNA from all treatments was extracted with QuickExtract (Lucigen) using the following cycles 1) at 65° C. for 15 min, 2) at 68° C. for 15 min, and 3) at 98° C. for 10 min and then held at 4° C. until use. The targeting window of 317 bp was amplified from the resulting extracted gDNA with Phusion Flash High-Fidelity PCR Master Mix (Thermo Fisher) using the following cycles 1) at 98° C. for 10 sec, 2) at 98° C. for 1 sec, 3) at 63° C. for 5 sec, 4) at 72° C. for 15 sec, and 5) at 72° C. for 1 min repeating steps 2-5 for 30 cycles then held at 4° C. Amplicons were visualized on 2% agarose gels before cleaning and concentrating with HighPrep Magnetic Beads (MagBio Genomics Inc.) with 1.8X bead volume to sample. Samples were eluted in water. INDELs were sequenced by NGS on a MiSeq with a v3 reagent kit (600-cycles; Table 8) and 5% phiX for 2×301 bp paired-end reads, with a minimum of 20,000 reads per sample. INDEL analysis was performed with a modified CRISPResso2 program (Clement et al., 2019; https://doi.org/10.1038/s41587-019-0032-3), and results are shown in Table 9 and FIG. 15B.

TABLE 8
Oligos used for NGS PCR1

| Oligo Name | Oligo Sequence (5′-3′) |
|---|---|
| 611F_NGS | GCTCTTCCGATCTNNNNNTGCACAGATATAAACACTTAA |
| | CGGG |
| 927R_NGS | GCTCTTCCGATCTNNNNNTTCAGCATTATAACTTACAGG |
| | CCT |

TABLE 9
Percent INDELs normalized to Apo conditions

| | Replicate1 | Replicate2 | Replicate3 | Average |
|---|---|---|---|---|
| RNP | % INDEL | % INDEL | % INDEL | % INDEL |
| 119-28 | 0.70 | 0.23 | 0.87 | 0.60 |
| sgRNA1_Mouse_Alb | | | | |
| 119-28 | 0.32 | 0.48 | 0.38 | 0.39 |
| sgRNA2_Mouse_Alb | | | | |
| 119-28 | 50.57 | 13.12 | 11.68 | 25.12 |
| sgRNA3_Mouse_Alb | | | | |
| 119-28 | 9.23 | 2.52 | 0.60 | 4.12 |
| sgRNA4_Mouse_Alb | | | | |

### Example 15—Buffer Optimization for MG119 Protein Purification (Prophetic)

Thus far, MG119 proteins have been purified in Nickel_A buffer. Nickel_A buffer is incompatible with downstream in vivo assays due to its high salinity, and rapid dilution into low-salt solutions induces protein precipitation. To optimize buffers for protein stability and downstream assay compatibility, MG119 nucleases are purified initially in high-salt buffers (750 mM NaCl) and gradually washed into a Nickel_A buffer variant with 200 mM NaCl and the zwitterionic amino acids L-arginine (50 mM) and L-glutamate (50 mM). On an empirical basis, various stabilizing sugars (ribose, sorbitol, mannitol, xylitol) are also added to the buffers to enhance protein stability in low salt buffers.

### Example 16—Fluorescence-Based Measurement of Nuclease Activity (Prophetic)

### Novel Cell Line Engineering

Current assays used to measure in vivo (i.e., in mammalian cell lines) nuclease activity require extensive data analysis and turnaround times of up to a week. To expedite evaluation of in vivo nuclease activity, an immortalized mammalian cell line is engineered to provide immediate data on editing of genomic DNA. K562 mammalian cells, grown in IMDM (Gibco #12440053)+10% FBS (Corning™ Regular Fetal Bovine Serum, MT35011CV), are used for this assay. K562 mammalian cells are transfected with 12 pmol Cas9 protein (IDT #1081058), 60 pmol sgRNA (Mali et al. Science, 2013 Feb. 15; 339(6121):823-6), and 1200 ng plasmid (pUC backbone) containing an expression sequence for an mMBP-(GGS)3-eGFP protein. Genomic integration of this construct results in constitutive expression under the synthetic MND promoter. Cells are left to grow for 6 days, passaging every 3 days. Monogenic cell lines are isolated from single cells by sorting individual GFP-expressing cells into a 96-well plate using a Sony MA900 Cell Sorter.

### Fluorescence-Based In Vivo Nuclease Activity Screen

Appropriate sgRNAs are designed to direct nuclease cleavage along the mMBP and eGFP genes, such that indel formation produces a frameshift mutation resulting in loss of fluorescence. MG119 RNP complexes are formed by combining 100 pmol protein and 200 pmol sgRNA and incubating at room temperature for ≥20 min in a final volume of 5 μL. K562 cells are washed in 1×PBS and resuspended in Nucleofector Solution (SF Cell Line 96-well Nucleofector™ Solution) with approximately 200,000 cells per well. Cells and RNP are combined in a Lonza 96-well nucleofection plate (SF Cell Line 96-well Nucleofector™ Kit, V4SC-2096) in a final volume of 25 μL, nucleofected (K562 cells, FF-120), and recovered in IMDM+10% FBS media. Cells are left to recover for 2-3 days at 37° C. To analyze, cells are washed twice with 1×PBS, then stained with 1× PBS+LIVE/DEAD Fixable Near-IR Dead Cell Stain Kit dye (ThermoFisher L10119) for 20 min at room temperature. Cells are washed once more with 1×PBS before being resuspended in 1×PBS and loaded into an Attune NxT, Acoustic Focusing Flow Cytometer (model AFC2) for fluorescence analysis. Positive unedited controls (nucleofected without RNP) and negative controls (non-fluorescent K562 cells) are used to establish positive and negative fluorescence gates, and cell populations are analyzed for loss-of-fluorescence in the GFP channel to evaluate in vivo nuclease activity.

### Example 17—Use for Epigenome Editing (Prophetic)

Epigenome editing is a gene modulation technique that comprises turning genes on or off constitutively or temporarily. Such techniques may use catalytically dead Cas9 (dCas9) fused to 3 proteins: Dnmt3A, Dnmt3L, and KRAB (e.g., as described in Nuñez et al. Cell 2021, 184(9), 2503-2519, which is incorporated herein by this reference in its entirety). Dnmt3A and Dnmt3L are DNA methyltransferases. The KRAB domain mediates histone methylation. The methylation of DNA and histones in the promoter region mediates constitutive gene repression. dCas9 and a guide RNA may recruit the DNA and histone methylation complex to the promoter region, requiring no nuclease activity. Together, Dnmt3A, Dnmt3L, and KRAB are 579 aa, and dCas9 is 1,368 aa. The fusion protein consists of 1,947 aa or 5,841 nucleotides, exceeding the adeno-associated virus vector (AAV) packaging limit (4.7 Kb). Therefore, there is a need to create more compact epigenome editors. Compact Type V nucleases from the MG119 family represent great candidates for use as the dead nuclease partners in technologies for epigenome editing. Due to their small size, ranging from 350 to 700 aa, when fused to DNA and histone methylation complexes, the size of the fusion proteins may range from, for example, about 929 to about 1,279 aa, or about 2787 to about 3837 nucleotides, allowing easy packaging in AAVs.

To test MG119 fusion proteins as epigenome editors, HEK293T cells expressing GFP under a chimeric promoter (GAPDH-Srnpn) are generated by lentiviral transduction. MG119 family guide RNAs targeting the chimeric promoter are designed. Guides are ordered from IDT, modifying the 5′ and 3′ nucleotides with 3 2′-O-methyl substituents and 3 phosphorothioate bonds for stability. Dead versions of MG119 nucleases are fused to DNA and histone methylation complexes (MG119 epigenome editors). The fusion proteins are cloned in mammalian expression plasmids under the CMV promoter. GFP expressing HEK293T cells are transfected with chemically synthesized guides and plasmids expressing MG119 epigenome editors. Transfected cells are analyzed by flow cytometry. Successful MG119 epigenome editors are determined by the loss of GFP fluorescence in transfected cells. MG119 epigenome editors are then used to target genes of therapeutic interest.

TABLE 10
Protein and nucleic acid sequences referred to herein

| Cat. | SEQ ID NO: | Description | Type |
|---|---|---|---|
| MG122 effectors | 1 | MG122-1 Effector | protein |
| MG122 effectors | 2 | MG122-2 Effector | protein |
| MG122 effectors | 3 | MG122-3 Effector | protein |
| MG122 effectors | 4 | MG122-4 Effector | protein |
| MG122 effectors | 5 | MG122-5 Effector | protein |
| MG120 effectors | 6 | MG120-1 Effector | protein |
| MG120 effectors | 7 | MG120-2 Effector | protein |
| MG120 effectors | 8 | MG120-3 Effector | protein |
| MG120 effectors | 9 | MG120-4 Effector | protein |
| MG120 effectors | 10 | MG120-5 Effector | protein |
| MG120 effectors | 11 | MG120-6 Effector | protein |
| MG120 effectors | 12 | MG120-7 Effector | protein |
| MG120 effectors | 13 | MG120-8 Effector | protein |
| MG120 effectors | 14 | MG120-9 Effector | protein |
| MG118 Effectors | 15 | MG118-1 Effector | protein |
| MG90 Effectors | 16 | MG90-3 Effector | protein |
| MG90 Effectors | 17 | MG90-5 Effector | protein |
| MG90 Effectors | 18 | MG90-6 Effector | protein |
| MG90 Effectors | 19 | MG90-7 Effector | protein |
| MG90 Effectors | 20 | MG90-8 Effector | protein |
| MG90 Effectors | 21 | MG90-16 Effector | protein |
| MG90 Effectors | 22 | MG90-17 Effector | protein |
| MG90 Effectors | 23 | MG90-18 Effector | protein |
| MG90 Effectors | 24 | MG90-19 Effector | protein |
| MG90 Effectors | 25 | MG90-20 Effector | protein |
| MG90 Effectors | 26 | MG90-21 Effector | protein |
| MG90 Effectors | 27 | MG90-22 Effector | protein |
| MG90 Effectors | 28 | MG90-23 Effector | protein |
| MG90 Effectors | 29 | MG90-24 Effector | protein |
| MG119 Effectors | 30 | MG119-1 Effector | protein |
| MG119 Effectors | 31 | MG119-2 Effector | protein |
| MG119 Effectors | 32 | MG119-3 Effector | protein |
| MG119 Effectors | 33 | MG119-4 Effector | protein |
| MG119 Effectors | 34 | MG119-5 Effector | protein |
| MG119 Effectors | 35 | MG119-6 Effector | protein |
| MG119 Effectors | 36 | MG119-7 Effector | protein |
| MG119 Effectors | 37 | MG119-8 Effector | protein |
| MG119 Effectors | 38 | MG119-9 Effector | protein |
| MG119 Effectors | 39 | MG119-10 Effector | protein |
| MG119 Effectors | 40 | MG119-11 Effector | protein |
| MG119 Effectors | 41 | MG119-12 Effector | protein |
| MG119 Effectors | 42 | MG119-13 Effector | protein |
| MG119 Effectors | 43 | MG119-14 Effector | protein |
| MG119 Effectors | 44 | MG119-15 Effector | protein |
| MG119 Effectors | 45 | MG119-16 Effector | protein |
| MG119 Effectors | 46 | MG119-17 Effector | protein |
| MG119 Effectors | 47 | MG119-18 Effector | protein |
| MG119 Effectors | 48 | MG119-19 Effector | protein |
| MG119 Effectors | 49 | MG119-20 Effector | protein |
| MG119 Effectors | 50 | MG119-21 Effector | protein |
| MG119 Effectors | 51 | MG119-22 Effector | protein |
| MG119 Effectors | 52 | MG119-23 Effector | protein |
| MG119 Effectors | 53 | MG119-24 Effector | protein |
| MG119 Effectors | 54 | MG119-25 Effector | protein |
| MG119 Effectors | 55 | MG119-26 Effector | protein |
| MG119 Effectors | 56 | MG119-27 Effector | protein |
| MG119 Effectors | 57 | MG119-28 Effector | protein |
| MG119 Effectors | 58 | MG119-29 Effector | protein |
| MG119 Effectors | 59 | MG119-30 Effector | protein |
| MG119 Effectors | 60 | MG119-31 Effector | protein |
| MG119 Effectors | 61 | MG119-32 Effector | protein |
| MG119 Effectors | 62 | MG119-33 Effector | protein |
| MG119 Effectors | 63 | MG119-34 Effector | protein |
| MG119 Effectors | 64 | MG119-35 Effector | protein |
| MG119 Effectors | 65 | MG119-36 Effector | protein |
| MG119 Effectors | 66 | MG119-37 Effector | protein |
| MG119 Effectors | 67 | MG119-38 Effector | protein |
| MG119 Effectors | 68 | MG119-39 Effector | protein |
| MG119 Effectors | 69 | MG119-40 Effector | protein |
| MG119 Effectors | 70 | MG119-41 Effector | protein |
| MG119 Effectors | 71 | MG119-42 Effector | protein |
| MG119 Effectors | 72 | MG119-43 Effector | protein |
| MG119 Effectors | 73 | MG119-44 Effector | protein |
| MG119 Effectors | 74 | MG119-45 Effector | protein |
| MG119 Effectors | 75 | MG119-46 Effector | protein |
| MG119 Effectors | 76 | MG119-47 Effector | protein |
| MG119 Effectors | 77 | MG119-48 Effector | protein |
| MG119 Effectors | 78 | MG119-49 Effector | protein |
| MG119 Effectors | 79 | MG119-50 Effector | protein |
| MG119 Effectors | 80 | MG119-51 Effector | protein |
| MG119 Effectors | 81 | MG119-52 Effector | protein |
| MG119 Effectors | 82 | MG119-53 Effector | protein |
| MG119 Effectors | 83 | MG119-54 Effector | protein |
| MG119 Effectors | 84 | MG119-55 Effector | protein |
| MG119 Effectors | 85 | MG119-56 Effector | protein |
| MG119 Effectors | 86 | MG119-57 Effector | protein |
| MG119 Effectors | 87 | MG119-58 Effector | protein |
| MG119 Effectors | 88 | MG119-59 Effector | protein |
| MG119 Effectors | 89 | MG119-61 Effector | protein |
| MG119 Effectors | 90 | MG119-62 Effector | protein |
| MG119 Effectors | 91 | MG119-63 Effector | protein |
| MG119 Effectors | 92 | MG119-64 Effector | protein |
| MG119 Effectors | 93 | MG119-65 Effector | protein |
| MG119 Effectors | 94 | MG119-66 Effector | protein |
| MG119 Effectors | 95 | MG119-67 Effector | protein |
| MG119 Effectors | 96 | MG119-68 Effector | protein |
| MG119 Effectors | 97 | MG119-69 Effector | protein |
| MG119 Effectors | 98 | MG119-70 Effector | protein |
| MG119 Effectors | 99 | MG119-71 Effector | protein |
| MG119 Effectors | 100 | MG119-72 Effector | protein |
| MG119 Effectors | 101 | MG119-73 Effector | protein |
| MG119 Effectors | 102 | MG119-74 Effector | protein |
| MG119 Effectors | 103 | MG119-75 Effector | protein |
| MG119 Effectors | 104 | MG119-76 Effector | protein |
| MG119 Effectors | 105 | MG119-77 Effector | protein |
| MG119 Effectors | 106 | MG119-78 Effector | protein |
| MG119 Effectors | 107 | MG119-79 Effector | protein |
| MG119 Effectors | 108 | MG119-80 Effector | protein |
| MG119 Effectors | 109 | MG119-81 Effector | protein |
| MG119 Effectors | 110 | MG119-83 Effector | protein |
| MG119 Effectors | 111 | MG119-84 Effector | protein |
| MG119 Effectors | 112 | MG119-85 Effector | protein |
| MG119 Effectors | 113 | MG119-86 Effector | protein |
| MG119 Effectors | 114 | MG119-87 Effector | protein |
| MG119 Effectors | 115 | MG119-88 Effector | protein |
| MG119 Effectors | 116 | MG119-89 Effector | protein |
| MG119 Effectors | 117 | MG119-90 Effector | protein |
| MG119 Effectors | 118 | MG119-91 Effector | protein |
| MG119 Effectors | 119 | MG119-92 Effector | protein |
| MG119 Effectors | 120 | MG119-93 Effector | protein |
| MG119 Effectors | 121 | MG119-94 Effector | protein |
| MG119 Effectors | 122 | MG119-95 Effector | protein |
| MG119 Effectors | 123 | MG119-96 Effector | protein |
| MG119 Effectors | 124 | MG119-97 Effector | protein |
| MG119 Effectors | 125 | MG119-98 Effector | protein |
| MG119 Effectors | 126 | MG119-99 Effector | protein |
| MG119 Effectors | 127 | MG119-100 Effector | protein |
| MG119 Effectors | 128 | MG119-101 Effector | protein |
| MG119 Effectors | 129 | MG119-102 Effector | protein |
| MG119 Effectors | 130 | MG119-103 Effector | protein |
| MG119 Effectors | 131 | MG119-104 Effector | protein |
| MG119 Effectors | 132 | MG119-105 Effector | protein |
| MG119 Effectors | 133 | MG119-106 Effector | protein |
| MG119 Effectors | 134 | MG119-107 Effector | protein |
| MG119 Effectors | 135 | MG119-108 Effector | protein |
| MG119 Effectors | 136 | MG119-109 Effector | protein |
| MG119 Effectors | 137 | MG119-110 Effector | protein |
| MG119 Effectors | 138 | MG119-111 Effector | protein |
| MG119 Effectors | 139 | MG119-112 Effector | protein |
| MG119 Effectors | 140 | MG119-113 Effector | protein |
| MG119 Effectors | 141 | MG119-114 Effector | protein |
| MG119 Effectors | 142 | MG119-115 Effector | protein |
| MG119 Effectors | 143 | MG119-116 Effector | protein |
| MG119 Effectors | 144 | MG119-117 Effector | protein |
| MG119 Effectors | 145 | MG119-118 Effector | protein |
| MG119 Effectors | 146 | MG119-119 Effector | protein |
| MG119 Effectors | 147 | MG119-120 Effector | protein |
| MG119 Effectors | 148 | MG119-121 Effector | protein |
| MG119 Effectors | 149 | MG119-122 Effector | protein |
| MG119 Effectors | 150 | MG119-123 Effector | protein |
| MG91B Effectors | 151 | MG91B-1 Effector | protein |
| MG91B Effectors | 152 | MG91B-2 Effector | protein |
| MG91B Effectors | 153 | MG91B-3 Effector | protein |
| MG91B Effectors | 154 | MG91B-4 Effector | protein |
| MG91B Effectors | 155 | MG91B-5 Effector | protein |
| MG91B Effectors | 156 | MG91B-6 Effector | protein |
| MG91B Effectors | 157 | MG91B-7 Effector | protein |
| MG91B Effectors | 158 | MG91B-8 Effector | protein |
| MG91B Effectors | 159 | MG91B-9 Effector | protein |
| MG91B Effectors | 160 | MG91B-10 Effector | protein |
| MG91B Effectors | 161 | MG91B-11 Effector | protein |
| MG91B Effectors | 162 | MG91B-12 Effector | protein |
| MG91B Effectors | 163 | MG91B-13 Effector | protein |
| MG91B Effectors | 164 | MG91B-14 Effector | protein |
| MG91B Effectors | 165 | MG91B-15 Effector | protein |
| MG91B Effectors | 166 | MG91B-16 Effector | protein |
| MG91B Effectors | 167 | MG91B-17 Effector | protein |
| MG91B Effectors | 168 | MG91B-18 Effector | protein |
| MG91B Effectors | 169 | MG91B-19 Effector | protein |
| MG91B Effectors | 170 | MG91B-20 Effector | protein |
| MG91B Effectors | 171 | MG91B-21 Effector | protein |
| MG91B Effectors | 172 | MG91B-22 Effector | protein |
| MG91B Effectors | 173 | MG91B-23 Effector | protein |
| MG91B Effectors | 174 | MG91B-24 Effector | protein |
| MG91B Effectors | 175 | MG91B-25 Effector | protein |
| MG91B Effectors | 176 | MG91B-26 Effector | protein |
| MG91B Effectors | 177 | MG91B-27 Effector | protein |
| MG91B Effectors | 178 | MG91B-28 Effector | protein |
| MG91B Effectors | 179 | MG91B-29 Effector | protein |
| MG91B Effectors | 180 | MG91B-30 Effector | protein |
| MG91B Effectors | 181 | MG91B-31 Effector | protein |
| MG91B Effectors | 182 | MG91B-32 Effector | protein |
| MG91B Effectors | 183 | MG91B-33 Effector | protein |
| MG91B Effectors | 184 | MG91B-34 Effector | protein |
| MG91B Effectors | 185 | MG91B-35 Effector | protein |
| MG91B Effectors | 186 | MG91B-36 Effector | protein |
| MG91B Effectors | 187 | MG91B-37 Effector | protein |
| MG91B Effectors | 188 | MG91B-38 Effector | protein |
| MG91B Effectors | 189 | MG91B-39 Effector | protein |
| MG91B Effectors | 190 | MG91B-40 Effector | protein |
| MG91B Effectors | 191 | MG91B-41 Effector | protein |
| MG91B Effectors | 192 | MG91B-42 Effector | protein |
| MG91B Effectors | 193 | MG91B-43 Effector | protein |
| MG91B Effectors | 194 | MG91B-44 Effector | protein |
| MG91B Effectors | 195 | MG91B-45 Effector | protein |
| MG91B Effectors | 196 | MG91B-46 Effector | protein |
| MG91B Effectors | 197 | MG91B-47 Effector | protein |
| MG91B Effectors | 198 | MG91B-48 Effector | protein |
| MG91B Effectors | 199 | MG91B-49 Effector | protein |
| MG91B Effectors | 200 | MG91B-50 Effector | protein |
| MG91B Effectors | 201 | MG91B-51 Effector | protein |
| MG91B Effectors | 202 | MG91B-52 Effector | protein |
| MG91B Effectors | 203 | MG91B-53 Effector | protein |
| MG91B Effectors | 204 | MG91B-54 Effector | protein |
| MG91B Effectors | 205 | MG91B-55 Effector | protein |
| MG91B Effectors | 206 | MG91B-56 Effector | protein |
| MG91B Effectors | 207 | MG91B-57 Effector | protein |
| MG91B Effectors | 208 | MG91B-58 Effector | protein |
| MG91B Effectors | 209 | MG91B-59 Effector | protein |
| MG91B Effectors | 210 | MG91B-60 Effector | protein |
| MG91B Effectors | 211 | MG91B-61 Effector | protein |
| MG91B Effectors | 212 | MG91B-62 Effector | protein |
| MG91B Effectors | 213 | MG91B-63 Effector | protein |
| MG91B Effectors | 214 | MG91B-64 Effector | protein |
| MG91B Effectors | 215 | MG91B-65 Effector | protein |
| MG91B Effectors | 216 | MG91B-66 Effector | protein |
| MG91B Effectors | 217 | MG91B-67 Effector | protein |
| MG91B Effectors | 218 | MG91B-68 Effector | protein |
| MG91B Effectors | 219 | MG91B-69 Effector | protein |
| MG91B Effectors | 220 | MG91B-70 Effector | protein |
| MG91B Effectors | 221 | MG91B-71 Effector | protein |
| MG91B Effectors | 222 | MG91B-72 Effector | protein |
| MG91B Effectors | 223 | MG91B-73 Effector | protein |
| MG91B Effectors | 224 | MG91B-74 Effector | protein |
| MG91B Effectors | 225 | MG91B-75 Effector | protein |
| MG91B Effectors | 226 | MG91B-76 Effector | protein |
| MG91B Effectors | 227 | MG91B-77 Effector | protein |
| MG91B Effectors | 228 | MG91B-78 Effector | protein |
| MG91B Effectors | 229 | MG91B-79 Effector | protein |
| MG91B Effectors | 230 | MG91B-80 Effector | protein |
| MG91B Effectors | 231 | MG91B-81 Effector | protein |
| MG91B Effectors | 232 | MG91B-82 Effector | protein |
| MG91B Effectors | 233 | MG91B-83 Effector | protein |
| MG91B Effectors | 234 | MG91B-84 Effector | protein |
| MG91B Effectors | 235 | MG91B-85 Effector | protein |
| MG91B Effectors | 236 | MG91B-86 Effector | protein |
| MG91B Effectors | 237 | MG91B-87 Effector | protein |
| MG91B Effectors | 238 | MG91B-88 Effector | protein |
| MG91B Effectors | 239 | MG91B-89 Effector | protein |
| MG91B Effectors | 240 | MG91B-90 Effector | protein |
| MG91B Effectors | 241 | MG91B-91 Effector | protein |
| MG91B Effectors | 242 | MG91B-92 Effector | protein |
| MG91B Effectors | 243 | MG91B-93 Effector | protein |
| MG91B Effectors | 244 | MG91B-94 Effector | protein |
| MG91B Effectors | 245 | MG91B-95 Effector | protein |
| MG91B Effectors | 246 | MG91B-96 Effector | protein |
| MG91B Effectors | 247 | MG91B-97 Effector | protein |
| MG91B Effectors | 248 | MG91B-98 Effector | protein |
| MG91B Effectors | 249 | MG91B-99 Effector | protein |
| MG91B Effectors | 250 | MG91B-100 Effector | protein |
| MG91B Effectors | 251 | MG91B-101 Effector | protein |
| MG91B Effectors | 252 | MG91B-102 Effector | protein |
| MG91B Effectors | 253 | MG91B-103 Effector | protein |
| MG91B Effectors | 254 | MG91B-104 Effector | protein |
| MG91B Effectors | 255 | MG91B-105 Effector | protein |
| MG91B Effectors | 256 | MG91B-106 Effector | protein |
| MG91B Effectors | 257 | MG91B-107 Effector | protein |
| MG91B Effectors | 258 | MG91B-108 Effector | protein |
| MG91B Effectors | 259 | MG91B-109 Effector | protein |
| MG91B Effectors | 260 | MG91B-110 Effector | protein |
| MG91B Effectors | 261 | MG91B-111 Effector | protein |
| MG91B Effectors | 262 | MG91B-112 Effector | protein |
| MG91B Effectors | 263 | MG91B-113 Effector | protein |
| MG91B Effectors | 264 | MG91B-114 Effector | protein |
| MG91B Effectors | 265 | MG91B-115 Effector | protein |
| MG91B Effectors | 266 | MG91B-116 Effector | protein |
| MG91B Effectors | 267 | MG91B-117 Effector | protein |
| MG91B Effectors | 268 | MG91B-118 Effector | protein |
| MG91B Effectors | 269 | MG91B-119 Effector | protein |
| MG91B Effectors | 270 | MG91B-120 Effector | protein |
| MG91B Effectors | 271 | MG91B-121 Effector | protein |
| MG91B Effectors | 272 | MG91B-122 Effector | protein |
| MG91B Effectors | 273 | MG91B-123 Effector | protein |
| MG91B Effectors | 274 | MG91B-124 Effector | protein |
| MG91B Effectors | 275 | MG91B-125 Effector | protein |
| MG91B Effectors | 276 | MG91B-126 Effector | protein |
| MG91B Effectors | 277 | MG91B-127 Effector | protein |
| MG91B Effectors | 278 | MG91B-128 Effector | protein |
| MG91B Effectors | 279 | MG91B-129 Effector | protein |
| MG91B Effectors | 280 | MG91B-130 Effector | protein |
| MG91B Effectors | 281 | MG91B-131 Effector | protein |
| MG91B Effectors | 282 | MG91B-132 Effector | protein |
| MG91B Effectors | 283 | MG91B-133 Effector | protein |
| MG91B Effectors | 284 | MG91B-134 Effector | protein |
| MG91B Effectors | 285 | MG91B-135 Effector | protein |
| MG91B Effectors | 286 | MG91B-136 Effector | protein |
| MG91B Effectors | 287 | MG91B-137 Effector | protein |
| MG91B Effectors | 288 | MG91B-138 Effector | protein |
| MG91B Effectors | 289 | MG91B-139 Effector | protein |
| MG91B Effectors | 290 | MG91B-140 Effector | protein |
| MG91B Effectors | 291 | MG91B-141 Effector | protein |
| MG91C Effectors | 292 | MG91C-1 Effector | protein |
| MG91C Effectors | 293 | MG91C-2 Effector | protein |
| MG91C Effectors | 294 | MG91C-3 Effector | protein |
| MG91C Effectors | 295 | MG91C-4 Effector | protein |
| MG91C Effectors | 296 | MG91C-5 Effector | protein |
| MG91C Effectors | 297 | MG91C-6 Effector | protein |
| MG91C Effectors | 298 | MG91C-7 Effector | protein |
| MG91C Effectors | 299 | MG91C-8 Effector | protein |
| MG91C Effectors | 300 | MG91C-9 Effector | protein |
| MG91C Effectors | 301 | MG91C-10 Effector | protein |
| MG91C Effectors | 302 | MG91C-11 Effector | protein |
| MG91C Effectors | 303 | MG91C-12 Effector | protein |
| MG91C Effectors | 304 | MG91C-13 Effector | protein |
| MG91C Effectors | 305 | MG91C-14 Effector | protein |
| MG91C Effectors | 306 | MG91C-15 Effector | protein |
| MG91C Effectors | 307 | MG91C-16 Effector | protein |
| MG91C Effectors | 308 | MG91C-17 Effector | protein |
| MG91C Effectors | 309 | MG91C-18 Effector | protein |
| MG91C Effectors | 310 | MG91C-19 Effector | protein |
| MG91C Effectors | 311 | MG91C-20 Effector | protein |
| MG91C Effectors | 312 | MG91C-21 Effector | protein |
| MG91C Effectors | 313 | MG91C-22 Effector | protein |
| MG91C Effectors | 314 | MG91C-23 Effector | protein |
| MG91C Effectors | 315 | MG91C-24 Effector | protein |
| MG91C Effectors | 316 | MG91C-25 Effector | protein |
| MG91C Effectors | 317 | MG91C-26 Effector | protein |
| MG91C Effectors | 318 | MG91C-27 Effector | protein |
| MG91A Effectors | 319 | MG91A-1 Effector | protein |
| MG126 Effectors | 320 | MG126-3 Effector | protein |
| MG126 Effectors | 321 | MG126-4 Effector | protein |
| MG126 Effectors | 322 | MG126-5 Effector | protein |
| MG126 Effectors | 323 | MG126-6 Effector | protein |
| MG126 Effectors | 324 | MG126-7 Effector | protein |
| MG126 Effectors | 325 | MG126-8 Effector | protein |
| MG119-3 effector intergenic | 326 | MG119-3_IG1 | nucleotide |
| region encoding potential | | | |
| tracrRNA | | | |
| MG119-3 effector intergenic | 327 | MG119-3_IG2 | nucleotide |
| region encoding potential | | | |
| tracrRNA | | | |
| MG119-3 effector intergenic | 328 | MG119-3_IG3 | nucleotide |
| region encoding potential | | | |
| tracrRNA | | | |
| MG119-4 effector intergenic | 329 | MG119-4_IG1 | nucleotide |
| region encoding potential | | | |
| tracrRNA | | | |
| MG119-4 effector intergenic | 330 | MG119-4_IG2 | nucleotide |
| region encoding potential | | | |
| tracrRNA | | | |
| MG119-4 effector intergenic | 331 | MG119-4_IG3 | nucleotide |
| region encoding potential | | | |
| tracrRNA | | | |
| MG119-4 effector intergenic | 332 | MG119-4_IG4 | nucleotide |
| region encoding potential | | | |
| tracrRNA | | | |
| MG120-1 effector intergenic | 333 | MG120-1_IG1 | nucleotide |
| region encoding potential | | | |
| tracrRNA | | | |
| MG120-1 effector intergenic | 334 | MG120-1_IG2 | nucleotide |
| region encoding potential | | | |
| tracrRNA | | | |
| MG120-1 effector intergenic | 335 | MG120-1_IG3 | nucleotide |
| region encoding potential | | | |
| tracrRNA | | | |
| MG119-1 effector intergenic | 336 | MG119-1_IG1 | nucleotide |
| region encoding potential | | | |
| tracrRNA | | | |
| MG119-1 effector intergenic | 337 | MG119-1_IG2 | nucleotide |
| region encoding potential | | | |
| tracrRNA | | | |
| MG119-1 effector intergenic | 338 | MG119-1_IG3 | nucleotide |
| region encoding potential | | | |
| tracrRNA | | | |
| MG119-1 effector intergenic | 339 | MG119-1_IG4 | nucleotide |
| region encoding potential | | | |
| tracrRNA | | | |
| MG119-1 effector intergenic | 340 | MG119-1_IG5 | nucleotide |
| region encoding potential | | | |
| tracrRNA | | | |
| MG119-2 effector intergenic | 341 | MG119-2_IG1 | nucleotide |
| region encoding potential | | | |
| tracrRNA | | | |
| MG119-2 effector intergenic | 342 | MG119-2_IG2 | nucleotide |
| region encoding potential | | | |
| tracrRNA | | | |
| MG119-5 effector intergenic | 343 | MG119-5_IG1 | nucleotide |
| region encoding potential | | | |
| tracrRNA | | | |
| MG119-5 effector intergenic | 344 | MG119-5_IG2 | nucleotide |
| region encoding potential | | | |
| tracrRNA | | | |
| MG119-5 effector intergenic | 345 | MG119-5_IG3 | nucleotide |
| region encoding potential | | | |
| tracrRNA | | | |
| MG90-3 effector intergenic | 346 | MG90-3_IG1 | nucleotide |
| region encoding potential | | | |
| tracrRNA | | | |
| MG90-3 effector intergenic | 347 | MG90-3_IG2 | nucleotide |
| region encoding potential | | | |
| tracrRNA | | | |
| MG119-3 effector intergenic | 348 | MG119-3_IG1_adapters | nucleotide |
| region encoding potential | | | |
| tracrRNA plus adapters | | | |
| MG119-3 effector intergenic | 349 | MG119-3_IG2_adapters | nucleotide |
| region encoding potential | | | |
| tracrRNA plus adapters | | | |
| MG119-3 effector intergenic | 350 | MG119-3_IG3_adapters | nucleotide |
| region encoding potential | | | |
| tracrRNA plus adapters | | | |
| MG119-4 effector intergenic | 351 | MG119-4_IG1_adapters | nucleotide |
| region encoding potential | | | |
| tracrRNA plus adapters | | | |
| MG119-4 effector intergenic | 352 | MG119-4_IG2_adapters | nucleotide |
| region encoding potential | | | |
| tracrRNA plus adapters | | | |
| MG119-4 effector intergenic | 353 | MG119-4_IG3_adapters | nucleotide |
| region encoding potential | | | |
| tracrRNA plus adapters | | | |
| MG119-4 effector intergenic | 354 | MG119-4_IG4_adapters | nucleotide |
| region encoding potential | | | |
| tracrRNA plus adapters | | | |
| MG120-1 effector intergenic | 355 | MG120-1_IG1_adapters | nucleotide |
| region encoding potential | | | |
| tracrRNA plus adapters | | | |
| MG120-1 effector intergenic | 356 | MG120-1_IG2_adapters | nucleotide |
| region encoding potential | | | |
| tracrRNA plus adapters | | | |
| MG120-1 effector intergenic | 357 | MG120-1_IG3_adapters | nucleotide |
| region encoding potential | | | |
| tracrRNA plus adapters | | | |
| MG119-1 effector intergenic | 358 | MG119-1_IG1_adapters | nucleotide |
| region encoding potential | | | |
| tracrRNA plus adapters | | | |
| MG119-1 effector intergenic | 359 | MG119-1_IG2_adapters | nucleotide |
| region encoding potential | | | |
| tracrRNA plus adapters | | | |
| MG119-1 effector intergenic | 360 | MG119-1_IG3_adapters | nucleotide |
| region encoding potential | | | |
| tracrRNA plus adapters | | | |
| MG119-1 effector intergenic | 361 | MG119-1_IG4_adapters | nucleotide |
| region encoding potential | | | |
| tracrRNA plus adapters | | | |
| MG119-1 effector intergenic | 362 | MG119-1_IG5_adapters | nucleotide |
| region encoding potential | | | |
| tracrRNA plus adapters | | | |
| MG119-2 effector intergenic | 363 | MG119-2_IG1_adapters | nucleotide |
| region encoding potential | | | |
| tracrRNA plus adapters | | | |
| MG119-2 effector intergenic | 364 | MG119-2_IG2_adapters | nucleotide |
| region encoding potential | | | |
| tracrRNA plus adapters | | | |
| MG119-5 effector intergenic | 365 | MG119-5_IG1_adapters | nucleotide |
| region encoding potential | | | |
| tracrRNA plus adapters | | | |
| MG119-5 effector intergenic | 366 | MG119-5_IG2_adapters | nucleotide |
| region encoding potential | | | |
| tracrRNA plus adapters | | | |
| MG119-5 effector intergenic | 367 | MG119-5_IG3_adapters | nucleotide |
| region encoding potential | | | |
| tracrRNA plus adapters | | | |
| MG90-3 effector intergenic | 368 | MG90-3_IG1_adapters | nucleotide |
| region encoding potential | | | |
| tracrRNA plus adapters | | | |
| MG90-3 effector intergenic | 369 | MG90-3_IG2_adapters | nucleotide |
| region encoding potential | | | |
| tracrRNA plus adapters | | | |
| MG119-3 minimal array with T7 | 370 | 119-3_5U40_31_F | nucleotide |
| promoter, two repeats in the | | | |
| forward orientation, and one | | | |
| spacer | | | |
| MG119-3 minimal array with T7 | 371 | 119-3_5U40_31_R | nucleotide |
| promoter, two repeats in the | | | |
| reverse orientation, and one | | | |
| spacer | | | |
| MG119-4 minimal array with T7 | 372 | 119-4_5U40_31_F | nucleotide |
| promoter, two repeats in the | | | |
| forward orientation, and one | | | |
| spacer | | | |
| MG119-4 minimal array with T7 | 373 | 119-4_5U40_31_R | nucleotide |
| promoter, two repeats in the | | | |
| reverse orientation, and one | | | |
| spacer | | | |
| MG120-1 minimal array with T7 | 374 | 120-1_5U40_37_F | nucleotide |
| promoter, two repeats in the | | | |
| forward orientation, and one | | | |
| spacer | | | |
| MG120-1 minimal array with T7 | 375 | 120-1_5U40_37_R | nucleotide |
| promoter, two repeats in the | | | |
| reverse orientation, and one | | | |
| spacer | | | |
| MG118-1 minimal array with T7 | 376 | 118-1_5U40_38_R | nucleotide |
| promoter, two repeats in the | | | |
| reverse orientation, and one | | | |
| spacer | | | |
| MG119-1 minimal array with T7 | 377 | 119-1_5U67_32_F | nucleotide |
| promoter, two repeats in the | | | |
| forward orientation, and one | | | |
| spacer | | | |
| MG119-1 minimal array with T7 | 378 | 119-1_5U67_32_R | nucleotide |
| promoter, two repeats in the | | | |
| reverse orientation, and one | | | |
| spacer | | | |
| MG119-2 minimal array with T7 | 379 | 119-2_5U40_28_F | nucleotide |
| promoter, two repeats in the | | | |
| forward orientation, and one | | | |
| spacer | | | |
| MG119-2 minimal array with T7 | 380 | 119-2_5U40_28_R | nucleotide |
| promoter, two repeats in the | | | |
| reverse orientation, and one | | | |
| spacer | | | |
| MG119-5 minimal array with T7 | 381 | 119-5_5U40_31_F | nucleotide |
| promoter, two repeats in the | | | |
| forward orientation, and one | | | |
| spacer | | | |
| MG119-5 minimal array with T7 | 382 | 119-5_5U40_31_R | nucleotide |
| promoter, two repeats in the | | | |
| reverse orientation, and one | | | |
| spacer | | | |
| MG90-3 minimal array with T7 | 383 | 90-3_5U67_37_F | nucleotide |
| promoter, two repeats in the | | | |
| forward orientation, and one | | | |
| spacer | | | |
| MG90-3 minimal array with T7 | 384 | 90-3_5U67_37_R | nucleotide |
| promoter, two repeats in the | | | |
| reverse orientation, and one | | | |
| spacer | | | |
| MG119-3 minimal array with T7 | 385 | 119-3_5U40_31_F_adapters | nucleotide |
| promoter, two repeats in the | | | |
| forward orientation, and one | | | |
| spacer plus adapters | | | |
| MG119-3 minimal array with T7 | 386 | 119-3_5U40_31_R_adapters | nucleotide |
| promoter, two repeats in the | | | |
| reverse orientation, and one | | | |
| spacer plus adapters | | | |
| MG119-4 minimal array with T7 | 387 | 119-4_5U40_31_F_adapters | nucleotide |
| promoter, two repeats in the | | | |
| forward orientation, and one | | | |
| spacer plus adapters | | | |
| MG119-4 minimal array with T7 | 388 | 119-4_5U40_31_R_adapters | nucleotide |
| promoter, two repeats in the | | | |
| reverse orientation, and one | | | |
| spacer plus adapters | | | |
| MG120-1 minimal array with T7 | 389 | 120-1_5U40_37_F_adapters | nucleotide |
| promoter, two repeats in the | | | |
| forward orientation, and one | | | |
| spacer plus adapters | | | |
| MG120-1 minimal array with T7 | 390 | 120-1_5U40_37_R_adapters | nucleotide |
| promoter, two repeats in the | | | |
| reverse orientation, and one | | | |
| spacer plus adapters | | | |
| MG118-1 minimal array with T7 | 391 | 118-1_5U40_38_R_adapters | nucleotide |
| promoter, two repeats in the | | | |
| reverse orientation, and one | | | |
| spacer plus adapters | | | |
| MG119-1 minimal array with T7 | 392 | 119-1_5U67_32_F_adapters | nucleotide |
| promoter, two repeats in the | | | |
| forward orientation, and one | | | |
| spacer plus adapters | | | |
| MG119-1 minimal array with T7 | 393 | 119-1_5U67_32_R_adapters | nucleotide |
| promoter, two repeats in the | | | |
| reverse orientation, and one | | | |
| spacer plus adapters | | | |
| MG119-2 minimal array with T7 | 394 | 119-2_5U40_28_F_adapters | nucleotide |
| promoter, two repeats in the | | | |
| forward orientation, and one | | | |
| spacer plus adapters | | | |
| MG119-2 minimal array with T7 | 395 | 119-2_5U40_28_R_adapters | nucleotide |
| promoter, two repeats in the | | | |
| reverse orientation, and one | | | |
| spacer plus adapters | | | |
| MG119-5 minimal array with T7 | 396 | 119-5_5U40_31_F_adapters | nucleotide |
| promoter, two repeats in the | | | |
| forward orientation, and one | | | |
| spacer plus adapters | | | |
| MG119-5 minimal array with T7 | 397 | 119-5_5U40_31_R_adapters | nucleotide |
| promoter, two repeats in the | | | |
| reverse orientation, and one | | | |
| spacer plus adapters | | | |
| MG90-3 minimal array with T7 | 398 | 90-3_5U67_37_F_adapters | nucleotide |
| promoter, two repeats in the | | | |
| forward orientation, and one | | | |
| spacer plus adapters | | | |
| MG90-3 minimal array with T7 | 399 | 90-3_5U67_37_R_adapters | nucleotide |
| promoter, two repeats in the | | | |
| reverse orientation, and one | | | |
| spacer plus adapters | | | |
| MG118-1 crRNA with trimmed | 400 | MG118-1_U40_18nt_target | nucleotide |
| repeat and 18 nt universal | | | |
| spacer, target sequence | | | |
| MG118-1 crRNA with trimmed | 401 | MG118-1_U67_18nt_target | nucleotide |
| repeat and 18 nt universal | | | |
| spacer, target sequence | | | |
| MG90-3 sgRNA with 10 RAR | 402 | 90-3_sgRNA_10bp_RAR_U40_24_target | nucleotide |
| and 24 nt universal spacer, target | | | |
| sequence | | | |
| MG90-3 sgRNA with 16 RAR | 403 | 90-3_sgRNA_16bp_RAR_U40_24_target | nucleotide |
| and 24 nt universal spacer, target | | | |
| sequence | | | |
| MG119-1 sgRNA with 10 RAR | 404 | 119-1_sgRNA_10bp_RAR_U40_24_target | nucleotide |
| and 24 nt universal spacer, target | | | |
| sequence | | | |
| MG119-1 sgRNA with 15 RAR | 405 | 119-1_sgRNA_15bp_RAR_U40_24_target | nucleotide |
| and 24 nt universal spacer, target | | | |
| sequence | | | |
| MG119-2 sgRNA with 10 RAR | 406 | 119-2_sgRNA_10bp_RAR_U40_24_target | nucleotide |
| and 24 nt universal spacer, target | | | |
| sequence | | | |
| MG119-2 sgRNA with 16 RAR | 407 | 119-2_sgRNA_16bp_RAR_U40_24_target | nucleotide |
| and 24 nt universal spacer, target | | | |
| sequence | | | |
| MG119-5 sgRNA with 9 RAR | 408 | 119-5_sgRNA_9bp_RAR_U40_24_target | nucleotide |
| and 24 nt universal spacer, target | | | |
| sequence | | | |
| MG119-5 sgRNA with 16 RAR | 409 | 119-5_sgRNA_16bp_RAR_U40_24_target | nucleotide |
| and 24 nt universal spacer, target | | | |
| sequence | | | |
| MG118-1 crRNA with trimmed | 410 | MG118-1_U40_18nt | nucleotide |
| repeat and 18 nt universal spacer | | | |
| MG118-1 crRNA with trimmed | 411 | MG118-1_U67_18nt | nucleotide |
| repeat and 18 nt universal spacer | | | |
| MG90-3 sgRNA with 10 RAR | 412 | 90-3_sgRNA_10bp_RAR_U40_24 | nucleotide |
| and 24 nt universal spacer | | | |
| MG90-3 sgRNA with 16 RAR | 413 | 90-3_sgRNA_16bp_RAR_U40_24 | nucleotide |
| and 24 nt universal spacer | | | |
| MG119-1 sgRNA with 10 RAR | 414 | 119-1_sgRNA_10bp_RAR_U40_24 | nucleotide |
| and 24 nt universal spacer | | | |
| MG119-1 sgRNA with 15 RAR | 415 | 119-1_sgRNA_15bp_RAR_U40_24 | nucleotide |
| and 24 nt universal spacer | | | |
| MG119-2 sgRNA with 10 RAR | 416 | 119-2_sgRNA_10bp_RAR_U40_24 | nucleotide |
| and 24 nt universal spacer | | | |
| MG119-2 sgRNA with 16 RAR | 417 | 119-2_sgRNA_16bp_RAR_U40_24 | nucleotide |
| and 24 nt universal spacer | | | |
| MG119-5 sgRNA with 9 RAR | 418 | 119-5_sgRNA_9bp_RAR_U40_24 | nucleotide |
| and 24 nt universal spacer | | | |
| MG119-5 sgRNA with 16 RAR | 419 | 119-5_sgRNA_16bp_RAR_U40_24 | nucleotide |
| and 24 nt universal spacer | | | |
| MG119 Effectors | 420 | MG119-124 Effector | protein |
| MG119 Effectors | 421 | MG119-125 Effector | protein |
| MG119 Effectors | 422 | MG119-126 Effector | protein |
| MG119 Effectors | 423 | MG119-127 Effector | protein |
| MG119 Effectors | 424 | MG119-128 Effector | protein |
| MG119 Effectors | 425 | MG119-129 Effector | protein |
| MG119 Effectors | 426 | MG119-130 Effector | protein |
| MG119 Effectors | 427 | MG119-131 Effector | protein |
| MG119 Effectors | 428 | MG119-132 Effector | protein |
| MG119 Effectors | 429 | MG119-133 Effector | protein |
| MG119 Effectors | 430 | MG119-134 Effector | protein |
| MG119 Effectors | 431 | MG119-135 Effector | protein |
| MG119-1 effectors sgRNA1 | 432 | MG119-1_sgRNA1 | Nucleotide |
| MG119-1 effectors PAM (5′) | 433 | MG119-1 PAM (5′) | Nucleotide |
| MG119-2 effectors sgRNA1 | 434 | MG119-2_sgRNA1_Mutant1 | Nucleotide |
| MG119-2 effectors PAM (5′) | 435 | MG119-2 PAM (5′) | Nucleotide |
| MG119-3 effectors sgRNA1 | 436 | MG119-3_sgRNA1_Mutant1 | Nucleotide |
| MG119-3 effectors PAM (5′) | 437 | MG119-3 PAM (5′) | Nucleotide |
| MG119-4 effectors sgRNA1 | 438 | MG119-4_sgRNA1 | Nucleotide |
| MG119-4 effectors PAM (5′) | 439 | MG119-4 PAM (5′) | Nucleotide |
| MG119-10 effectors sgRNA1 | 440 | MG119-10_sgRNA1 | Nucleotide |
| MG119-10 effectors PAM (5′) | 441 | MG119-10 PAM (5′) | Nucleotide |
| MG119-19 effectors sgRNA1 | 442 | MG119-19_sgRNA1 | Nucleotide |
| MG119-19 effectors PAM (5′) | 443 | MG119-19 PAM (5′) | Nucleotide |
| MG119-27 effectors sgRNA2 | 444 | MG119-27_sgRNA2_Mutant2 | Nucleotide |
| MG119-27 effectors PAM (5′) | 445 | MG119-27 PAM (5′) | Nucleotide |
| MG119-28 effectors sgRNA2 | 446 | MG119-28_sgRNA2 | Nucleotide |
| MG119-28 effectors PAM (5′) | 447 | MG119-28 PAM (5′) | Nucleotide |
| MG119-32 effectors sgRNA1 | 448 | MG119-32_sgRNA1 | Nucleotide |
| MG119-32 effectors PAM (5′) | 449 | MG119-32 PAM (5′) | Nucleotide |
| MG119-54 effectors sgRNA1 | 450 | MG119-54_sgRNA1 | Nucleotide |
| MG119-54 effectors PAM (5′) | 451 | MG119-54 PAM (5′) | Nucleotide |
| MG119-64 effectors sgRNA2 | 452 | MG119-64_sgRNA2 | Nucleotide |
| MG119-64 effectors PAM (5′) | 453 | MG119-64 PAM (5′) | Nucleotide |
| MG119-72 effectors sgRNA1 | 454 | MG119-72_sgRNA1 | Nucleotide |
| MG119-72 effectors PAM (5′) | 455 | MG119-72 PAM (5′) | Nucleotide |
| MG119-83 effectors sgRNA1 | 456 | MG119-83_sgRNA1 | Nucleotide |
| MG119-83 effectors PAM (5′) | 457 | MG119-83 PAM (5′) | Nucleotide |
| MG119-97 effectors sgRNA1 | 458 | MG119-97_sgRNA1_Mutant1 | Nucleotide |
| MG119-97 effectors PAM (5′) | 459 | MG119-97_PAM (5′) | Nucleotide |
| MG119-109 effectors sgRNA1 | 460 | MG119-109_sgRNA1 | Nucleotide |
| MG119-109 effectors PAM (5′) | 461 | MG119-109 PAM (5′) | Nucleotide |
| MG119-118 effectors sgRNA1 | 462 | MG119-118_sgRNA1_Mutant2 | Nucleotide |
| MG119-118 effectors PAM (5′) | 463 | MG119-118 PAM (5′) | Nucleotide |
| MG119-121 effectors sgRNA1 | 464 | MG119-121_sgRNA1_Mutant1 | Nucleotide |
| MG119-121 effectors PAM (5′) | 465 | MG119-121 PAM (5′) | Nucleotide |
| MG119-125 effectors sgRNA1 | 466 | MG119-125_sgRNA1 | Nucleotide |
| MG119-125 effectors PAM (5′) | 467 | MG119-125 PAM (5′) | Nucleotide |
| MG119-128 effectors sgRNA1 | 468 | MG119-128_sgRNA2_Mutant1 | Nucleotide |
| MG119-128 effectors PAM (5′) | 469 | MG119-128 PAM (5′) | Nucleotide |
| MG119-129 effectors sgRNA1 | 470 | MG119-129_sgRNA1_Mutant1 | Nucleotide |
| MG119-129 effectors PAM (5′) | 471 | MG119-129 PAM (5′) | Nucleotide |
| MG119-133 effectors sgRNA1 | 472 | MG119-133_sgRNA1_Mutant1 | Nucleotide |
| MG119-133 effectors PAM (5′) | 473 | MG119-133 PAM (5′) | Nucleotide |
| MG119-136 effectors sgRNA1 | 474 | MG119-136_sgRNA1_Mutant2 | Nucleotide |
| MG119-136 effectors PAM (5′) | 475 | MG119-136 PAM (5′) | Nucleotide |
| MG119-136 active effectors | 476 | MG119-136 effector | Protein |
| MG119-139 effectors | 477 | MG119-139 effector | Protein |
| MG119-140 effectors | 478 | MG119-140 effector | Protein |
| MG119-141 effectors | 479 | MG119-141 effector | Protein |
| MG119-142 effectors | 480 | MG119-142 effector | Protein |
| MG119-143 effectors | 481 | MG119-143 effector | Protein |
| MG119-144 effectors | 482 | MG119-144 effector | Protein |
| MG119-145 effectors | 483 | MG119-145 effector | Protein |
| MG119-146 effectors | 484 | MG119-146 effector | Protein |
| MG119-147 effectors | 485 | MG119-147 effector | Protein |
| MG119-148 effectors | 486 | MG119-148 effector | Protein |
| MG119-149 effectors | 487 | MG119-149 effector | Protein |
| MG119-150 effectors | 488 | MG119-150 effector | Protein |
| MG119-151 effectors | 489 | MG119-151 effector | Protein |
| MG119-152 effectors | 490 | MG119-152 effector | Protein |
| MG119-153 effectors | 491 | MG119-153 effector | Protein |
| MG119-154 effectors | 492 | MG119-154 effector | Protein |
| MG119-155 effectors | 493 | MG119-155 effector | Protein |
| MG119-156 effectors | 494 | MG119-156 effector | Protein |
| MG119-157 effectors | 495 | MG119-157 effector | Protein |
| MG119-158 effectors | 496 | MG119-158 effector | Protein |
| MG119-159 effectors | 497 | MG119-159 effector | Protein |
| MG119-160 effectors | 498 | MG119-160 effector | Protein |
| MG119-161 effectors | 499 | MG119-161 effector | Protein |
| MG119-162 effectors | 500 | MG119-162 effector | Protein |
| MG119-163 effectors | 501 | MG119-163 effector | Protein |
| MG119-164 effectors | 502 | MG119-164 effector | Protein |
| MG119-165 effectors | 503 | MG119-165 effector | Protein |
| MG119-166 effectors | 504 | MG119-166 effector | Protein |
| MG119-167 effectors | 505 | MG119-167 effector | Protein |
| MG119-168 effectors | 506 | MG119-168 effector | Protein |
| MG119-169 effectors | 507 | MG119-169 effector | Protein |
| MG119-170 effectors | 508 | MG119-170 effector | Protein |
| MG119-171 effectors | 509 | MG119-171 effector | Protein |
| MG119-172 effectors | 510 | MG119-172 effector | Protein |
| MG119-173 effectors | 511 | MG119-173 effector | Protein |
| MG119-174 effectors | 512 | MG119-174 effector | Protein |
| MG119-175 effectors | 513 | MG119-175 effector | Protein |
| MG119-176 effectors | 514 | MG119-176 effector | Protein |
| MG119-177 effectors | 515 | MG119-177 effector | Protein |
| MG119-178 effectors | 516 | MG119-178 effector | Protein |
| MG119-179 effectors | 517 | MG119-179 effector | Protein |
| MG119-180 effectors | 518 | MG119-180 effector | Protein |
| MG119-181 effectors | 519 | MG119-181 effector | Protein |
| MG119-182 effectors | 520 | MG119-182 effector | Protein |
| MG119-183 effectors | 521 | MG119-183 effector | Protein |
| MG119-184 effectors | 522 | MG119-184 effector | Protein |
| MG119-185 effectors | 523 | MG119-185 effector | Protein |
| MG119-186 effectors | 524 | MG119-186 effector | Protein |
| MG119-187 effectors | 525 | MG119-187 effector | Protein |
| MG119-188 effectors | 526 | MG119-188 effector | Protein |
| MG119-189 effectors | 527 | MG119-189 effector | Protein |
| MG119-190 effectors | 528 | MG119-190 effector | Protein |
| MG119-191 effectors | 529 | MG119-191 effector | Protein |
| MG119-192 effectors | 530 | MG119-192 effector | Protein |
| MG119-193 effectors | 531 | MG119-193 effector | Protein |
| MG119-194 effectors | 532 | MG119-194 effector | Protein |
| MG119-195 effectors | 533 | MG119-195 effector | Protein |
| MG119-196 effectors | 534 | MG119-196 effector | Protein |
| MG119-197 effectors | 535 | MG119-197 effector | Protein |
| MG119-198 effectors | 536 | MG119-198 effector | Protein |
| MG119-199 effectors | 537 | MG119-199 effector | Protein |
| MG119-200 effectors | 538 | MG119-200 effector | Protein |
| MG119-201 effectors | 539 | MG119-201 effector | Protein |
| MG119-202 effectors | 540 | MG119-202 effector | Protein |
| MG119-203 effectors | 541 | MG119-203 effector | Protein |
| MG119-204 effectors | 542 | MG119-204 effector | Protein |
| MG119-205 effectors | 543 | MG119-205 effector | Protein |
| MG119-206 effectors | 544 | MG119-206 effector | Protein |
| MG119-207 effectors | 545 | MG119-207 effector | Protein |
| MG119-208 effectors | 546 | MG119-208 effector | Protein |
| MG119-209 effectors | 547 | MG119-209 effector | Protein |
| MG119-210 effectors | 548 | MG119-210 effector | Protein |
| MG119-211 effectors | 549 | MG119-211 effector | Protein |
| MG119-212 effectors | 550 | MG119-212 effector | Protein |
| MG119-213 effectors | 551 | MG119-213 effector | Protein |
| MG119-214 effectors | 552 | MG119-214 effector | Protein |
| MG119-215 effectors | 553 | MG119-215 effector | Protein |
| MG119-216 effectors | 554 | MG119-216 effector | Protein |
| MG119-217 effectors | 555 | MG119-217 effector | Protein |
| MG119-218 effectors | 556 | MG119-218 effector | Protein |
| MG119-219 effectors | 557 | MG119-219 effector | Protein |
| MG119-220 effectors | 558 | MG119-220 effector | Protein |
| MG119-221 effectors | 559 | MG119-221 effector | Protein |
| MG119-222 effectors | 560 | MG119-222 effector | Protein |
| MG119-223 effectors | 561 | MG119-223 effector | Protein |
| MG119-224 effectors | 562 | MG119-224 effector | Protein |
| MG119-225 effectors | 563 | MG119-225 effector | Protein |
| MG119-226 effectors | 564 | MG119-226 effector | Protein |
| MG119-227 effectors | 565 | MG119-227 effector | Protein |
| MG119-228 effectors | 566 | MG119-228 effector | Protein |
| MG119-229 effectors | 567 | MG119-229 effector | Protein |
| MG119-230 effectors | 568 | MG119-230 effector | Protein |
| MG119-231 effectors | 569 | MG119-231 effector | Protein |
| MG119-232 effectors | 570 | MG119-232 effector | Protein |
| MG119-233 effectors | 571 | MG119-233 effector | Protein |
| MG119-234 effectors | 572 | MG119-234 effector | Protein |
| MG119-235 effectors | 573 | MG119-235 effector | Protein |
| MG119-236 effectors | 574 | MG119-236 effector | Protein |
| MG119-237 effectors | 575 | MG119-237 effector | Protein |
| MG119-238 effectors | 576 | MG119-238 effector | Protein |
| MG119-239 effectors | 577 | MG119-239 effector | Protein |
| MG119-240 effectors | 578 | MG119-240 effector | Protein |
| MG119-241 effectors | 579 | MG119-241 effector | Protein |
| MG119-242 effectors | 580 | MG119-242 effector | Protein |
| MG119-243 effectors | 581 | MG119-243 effector | Protein |
| MG119-244 effectors | 582 | MG119-244 effector | Protein |
| MG119-245 effectors | 583 | MG119-245 effector | Protein |
| MG119-246 effectors | 584 | MG119-246 effector | Protein |
| MG119-247 effectors | 585 | MG119-247 effector | Protein |
| MG119-248 effectors | 586 | MG119-248 effector | Protein |
| MG119-249 effectors | 587 | MG119-249 effector | Protein |
| MG119-250 effectors | 588 | MG119-250 effector | Protein |
| MG119-251 effectors | 589 | MG119-251 effector | Protein |
| MG119-252 effectors | 590 | MG119-252 effector | Protein |
| MG119-253 effectors | 591 | MG119-253 effector | Protein |
| MG119-254 effectors | 592 | MG119-254 effector | Protein |
| MG119-255 effectors | 593 | MG119-255 effector | Protein |
| MG119-256 effectors | 594 | MG119-256 effector | Protein |
| MG119-257 effectors | 595 | MG119-257 effector | Protein |
| MG119-258 effectors | 596 | MG119-258 effector | Protein |
| MG119-259 effectors | 597 | MG119-259 effector | Protein |
| MG119-260 effectors | 598 | MG119-260 effector | Protein |
| MG119-261 effectors | 599 | MG119-261 effector | Protein |
| MG119-262 effectors | 600 | MG119-262 effector | Protein |
| MG119-263 effectors | 601 | MG119-263 effector | Protein |
| MG119-264 effectors | 602 | MG119-264 effector | Protein |
| MG119-265 effectors | 603 | MG119-265 effector | Protein |
| MG119-266 effectors | 604 | MG119-266 effector | Protein |
| MG119-267 effectors | 605 | MG119-267 effector | Protein |
| MG119-268 effectors | 606 | MG119-268 effector | Protein |
| MG119-269 effectors | 607 | MG119-269 effector | Protein |
| MG119-270 effectors | 608 | MG119-270 effector | Protein |
| MG119-271 effectors | 609 | MG119-271 effector | Protein |
| MG119-272 effectors | 610 | MG119-272 effector | Protein |
| MG119-273 effectors | 611 | MG119-273 effector | Protein |
| MG119-274 effectors | 612 | MG119-274 effector | Protein |
| MG119-275 effectors | 613 | MG119-275 effector | Protein |
| MG119-276 effectors | 614 | MG119-276 effector | Protein |
| MG119-277 effectors | 615 | MG119-277 effector | Protein |
| MG119-278 effectors | 616 | MG119-278 effector | Protein |
| MG119-279 effectors | 617 | MG119-279 effector | Protein |
| MG119-280 effectors | 618 | MG119-280 effector | Protein |
| MG119-281 effectors | 619 | MG119-281 effector | Protein |
| MG119-282 effectors | 620 | MG119-282 effector | Protein |
| MG119-283 effectors | 621 | MG119-283 effector | Protein |
| MG119-284 effectors | 622 | MG119-284 effector | Protein |
| MG119-285 effectors | 623 | MG119-285 effector | Protein |
| MG119-286 effectors | 624 | MG119-286 effector | Protein |
| sgRNAs | 625 | 119-28 sgRNA1_Mouse_Alb | Nucleotide |
| | | | (RNA) |
| sgRNAs | 626 | 119-28 sgRNA2_Mouse Alb | Nucleotide |
| | | | (RNA) |
| sgRNAs | 627 | 119-28 sgRNA3_Mouse_Alb | Nucleotide |
| | | | (RNA) |
| sgRNAs | 628 | 119-28 sgRNA4_Mouse_Alb | Nucleotide |
| | | | (RNA) |
| MG119 effectors | 629 | MG119-137 Effector | protein |

TABLE 11
Protein and nucleic acid sequences referred to herein

| | SEQ | | | | Other | |
|---|---|---|---|---|---|---|
| Cat. | ID NO: | Description | Type | Organism | Information | Sequence |
| MG119 | 629 | MG119-137 | protein | unknown | uncultivated | MSKDKYVITRKIKLLPVGG |
| effectors | | Effector | | | organism | ENEVDRVYDFIRNGQYSQY |
| | | | | | | QALNLLMGQLASKYYDCK |
| | | | | | | KDLSSAEFKDAQKSILSNSN |
| | | | | | | PNLCDIEFVKGCDTKSAVV |
| | | | | | | QKVRQDFSTAIKNGLPRGE |
| | | | | | | RNITNYKRTVPLITRGRDLV |
| | | | | | | FVHGYENYTEFLDNLYTDR |
| | | | | | | NLKVFIKWVNKIQFKIVFG |
| | | | | | | NPYKSAELRSVVQNIFEERY |
| | | | | | | KINGSSICIDDDDIILNLSLT |
| | | | | | | MPKEIKELDESKVVGVDLG |
| | | | | | | IAIPAVCALNTNSYSRKSIGS |
| | | | | | | ADDFLRVRTKIRAQRRRLQ |
| | | | | | | KSLSQTSGGHGRKKKLRAL |
| | | | | | | DKFSEYEKHWVQNYNHYV |
| | | | | | | SKQVVDFAIKNNAKYINLE |
| | | | | | | DLEGYGEEEKNKFILSNWS |
| | | | | | | YYQLQQYIAYKAEKYGIEV |
| | | | | | | RKINPYHTSQVCSCCGHWE |
| | | | | | | SGQRVNQKTFICKNPECEN |
| | | | | | | FGEEVNADFNAARNIALST |
| | | | | | | NWSDIDEKKNKKNKKK |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

The invention claimed is:

1.-140. (canceled)

141. An engineered nuclease system comprising:
(a) an endonuclease or a nucleic acid encoding the endonuclease, wherein the endonuclease is a class 2, type V endonuclease, wherein the endonuclease comprises a RuvCI domain, RuvCII domain, or RuvCIII domain comprising an amino acid sequence having at least 75% sequence identity to RuvCI domain, RuvCII domain, or RuvCIII domain, respectively, of SEQ ID NO: 57; and
(b) an engineered guide ribonucleic acid or a nucleic acid encoding the engineered guide ribonucleic acid, wherein the engineered guide ribonucleic acid is configured to form a complex with the endonuclease, wherein the engineered guide ribonucleic acid comprises a spacer sequence configured to hybridize to a target nucleic acid sequence.

142. The engineered nuclease system of claim 141, wherein the RuvCI domain, the RuvCII domain, or the RuvCIII domain of the endonuclease comprises an amino acid sequence having at least 90% sequence identity to the RuvCI domain, the RuvCII domain, or the RuvCIII domain, respectively, of SEQ ID NO: 57.

143. The engineered nuclease system of claim 142, wherein the RuvCI domain, the RuvCII, or the RuvCIII domain of the endonuclease comprises the amino acid sequence of the RuvCI domain, the RuvCII domain, or the RuvCIII domain, respectively, of SEQ ID NO: 57.

144. The engineered nuclease system of claim 141, wherein the endonuclease comprises a WED II domain comprising an amino acid sequence having at least 75% sequence identity to the WED II domain of SEQ ID NO: 57.

145. The engineered nuclease system of claim 144, wherein the WED II domain comprises an amino acid sequence having at least 90% sequence identity to the WED II domain of SEQ ID NO: 57.

146. The engineered nuclease system of claim 145, wherein the WED II domain comprises an amino acid sequence having the amino acid sequence of the WED II domain of SEQ ID NO: 57.

147. The engineered nuclease of claim 141, wherein the endonuclease is configured to be selective for a protospacer adjacent motif sequence comprising 5′-TTR-3′.

148. The engineered nuclease system of claim 141, wherein the endonuclease comprises an amino acid sequence having at least 90% sequence identity to SEQ ID NO: 57.

149. The engineered nuclease system of claim 148, wherein the endonuclease comprises the amino acid sequence of SEQ ID NO: 57.

150. The engineered nuclease system of claim 141, wherein the engineered guide ribonucleic acid comprises a sequence having at least 80% sequence identity to non-degenerate nucleotides of SEQ ID NO: 446.

151. The engineered nuclease system of claim 150, wherein the engineered guide ribonucleic acid comprises a nucleotide sequence having at least 90% sequence identity to non-degenerate nucleotides of SEQ ID NO: 446.

152. The engineered nuclease system of claim 151, wherein the engineered guide ribonucleic acid comprises the nucleotide sequence of SEQ ID NO: 446.

153. The engineered nuclease system of claim 148, wherein the endonuclease comprises the amino acid sequence of SEQ ID NO: 57 and wherein the engineered guide ribonucleic acid comprises the nucleotide sequence of SEQ ID NO: 446.

154. A method of modifying a target nucleic acid locus, the method comprising delivering to the target nucleic acid locus:
(a) an endonuclease or a nucleic acid encoding the endonuclease, wherein the endonuclease is a class 2, type V endonuclease, wherein the endonuclease comprises a RuvCI domain, RuvCII domain, or RuvCIII domain, comprising an amino acid sequence having at least 75% sequence identity to RuvCI domain, RuvCII domain, or RuvCIII domain, respectively, of SEQ ID NO: 57; and
(b) an engineered guide ribonucleic acid or a nucleic acid encoding the engineered guide ribonucleic acid, wherein the engineered guide ribonucleic acid is configured to form a complex with the endonuclease, and the engineered guide ribonucleic acid comprises a spacer sequence configured to hybridize to a eukaryotic target nucleic acid sequence,
wherein the complex modifies the target nucleic acid locus.

155. The method of claim 154, wherein the RuvCI domain, the RuvCII domain, or the RuvCIII domain comprises an amino acid sequence having at least 90% sequence identity to the RuvCI domain, the RuvCII domain, or the RuvCIII domain, respectively, of SEQ ID NO: 57.

156. The method of claim 154, wherein the endonuclease comprises a WED II domain comprising an amino acid sequence having at least 75% sequence identity to the WED II domain of SEQ ID NO: 57.

157. The method of claim 154, wherein the endonuclease comprises an amino acid sequence having at least 90% sequence identity to SEQ ID NO: 57.

158. The method of claim 157, wherein the endonuclease comprises the amino acid sequence of SEQ ID NO: 57.

159. The method of claim 154, wherein the engineered guide ribonucleic acid comprises a sequence having at least 80% sequence identity to non-degenerate nucleotides of SEQ ID NO: 446.

160. The method of claim 159, wherein the engineered guide ribonucleic acid comprises the nucleotide sequence of SEQ ID NO: 446.


SEQUENCE LISTING (628 sequences)

SEQ ID NO: 1 - protein, 924 aa, unidentified
  REGION 1..924: note = uncultivated organism
  REGION 1..924: note = MG122-1 Effector
MKLNHLSIAQRMEGAENIHELKENKKTISCGCAAMVDTIESADYIIRLHKAAIAAQNWTVQELGKIMFLPATFISGEGIAEKVTIFDLLSMNKASHKAFASKFSNNGEGFTGTRPDGTPFRTTKSFLGYLSREITSNENSPFCTDMQKRIITSLPEEFQGTASVFIRESIIKIAGSKIEAAEQKKMGPYDNGHAATWCMTDGELSEYIAETEKSAETKRDDEKKTPKKQDNSRQDNTWKRSVRFALADLDTITASITGFAGISSTELAILKEQLLITGPDPHAVDNGRVPGPVVWPAGRKVKVGNEVVTRNVKRPEWDGVFKAVYLNALDRFKTAVIENLPNITRGGLPIPVNIDYSERDERIAAGKKYDSKLIPIHLPMNKSCKVAIPEIFIKTRSGHSTDGYFPHNFTEMVPVTKAICCTLRLPDGCTPETLSSVNSGLIAQISYETVTTAVRMRPKADIPREKILGIDVNSATFKYNTSAGRLQAGQDMVDWHEAIAYFQGICPNDFLFNMGKYCTFEHIHTRICAEEYMASCKARLFSKASKQVDDILAQFRSGEYGIAMMPTLALLPTVFKPDTREVLPKDPMFNLFRALIERKGADGKTPYYSENARDTISHTRRWRHEIALALSAKLEYRDKAITWQADHDPVAEPFTNTEEAASILDRMTAAVNSAKQIEEHILSRSLFSYETVSRFTIYAVEDIDFDCLKDKSVSFMSLASTLRQIPALASGCSVAAQEASGNVRVKLDGGTGAEEAVTEIRKWFAGTSHRGQWSLETAEAVSAGEIRMKCSITRKGRIDRAVATVPNLLTKVMRLPEVKNTVERLCVKNATPFVSVDPRSTSLTCSACGARTTKEACKKGEPFCKETGRPFRLGERFVCHKCGREINANWNAAINVAGRASFIMANAKNKKKLAEPSDNSLPGM

SEQ ID NO: 2 - protein, 913 aa, unidentified
  REGION 1..913: note = uncultivated organism
  REGION 1..913: note = MG122-2 Effector
MKHLTVKDRTKGKSGSIFKIDTTTETVSCGCPAMVDSVESAEYLARLYTAAINAQNYAISMVRDMLFVPAKFVAPDGHAEKVTPFDLFAMNGPTARAFFERFATVETVGGEKTSYFAGVRPDRKKFRTVGSFMQYLSREIFSDTSSPFGKALKTKIIESMPGEFRSRSVSFVNESVIKVAIPKVACAIRKKVEQYTGSCVEPWCLSGEEAHKASGKSSGKRADHTWKRIARDVCDSLVDIDPLDKVTLYAQLNAVGPRCHEFINRKNPVVGHWPENATVRIPGLARKMPLDRPSWNGQFRRVYGQAIEKFMDEVAARLPKITGGSLVVLGEVDGDGRESRIRRGDKFDCVFNVSHVPYDKEKFVPSVKLYLQTRTRHTKDGYFPSNFDETLGTNKVVQCSLRFPSEFDVQSLTRRSSKLIAQLSYERVSDKVRFAGPCELSDKDVVGIDANSSTFKINTTIPPKRKCSDIPDWHEAFLAFRESNRDHFVFNAKQYADGLQTDMQDGLLRSTLMERFAVQVDSIVESAKSGKYGLCLFPTLGIPLMLFENNLYLAGENPGKDPLSALFTFMANRKDANGNPFYGPAAREAIGHTRAWRRATAAALAEKLRYITESRVWQETHDPVNSPFGDTGRAAELSERCSAAMRTARQIEEHVLSKTLFSKNTVSKFKLFALEELDFDGMRSKDGGFVSLGATLRSMLGDMYKVGPCREASGYAEIELPYDDKPAAFTKRLKELFKERKGKCNWEIVKVSYSLKIVGKFEKNNCTRVETAKTYNVAKVKFRLTRKGFCAMNAGLVPAALTKVLHLPELQHTMERLCAKNATSYISVNPKYTSCTCSKCQAKTGKIMCGKGESYCKEMHVPYRRGEYFHCYQCGTEMNANFNAALNVRGSGIRAIGSIGKKKTESGGRRMKK

SEQ ID NO: 3 - protein, 801 aa, unidentified
  REGION 1..801: note = uncultivated organism
  REGION 1..801: note = MG122-3 Effector
MQKTSTSVVYTNVTKKGKPITRTRTIKCPVAFVSKKDAEWVMQLGVATKITMNHVLGILRDKFFDTMTIIAGGKEQVITLFDLFNSKSGLEGPFVYNGNTTCESIVENPVRELLNPKCPLIGVTTKEIIHFMETQLPDGVYVPGKFVKESVYKQVQGTVRGAVTKKLNQEKGKAKKDKTWQKCCRKAAEVHTKTDDAAKELSLQLRIVGVQIYAMNNAGTPQIDEWPKECDVPKPEINEAFIAAYKDAIALFKTAFVEAFPEIRSQSLLYCPMFKLDWGTRDTAKFYDIQGKITGIPNIENKKRYSVKVYIRTVSGHSDNYFPKDLQESLGNESVLGIKFPEVITHEDITSVDGVRATKCNTLGYLSVTCPHMKTKFNADGISTDDGVGIDLNVASFFMNTTIKMDETDGAVDWPEAVNEFRSQNPTAYPFTMPYPRSVKDLNLIADNVNEKDRLLRVIPLVGLRDGKPSDEKHGWKASPDPLSMLFTWMLKRTDNNGNPFYSDEQRAHIGHTRDYRKLIRSDTANRLHYFHEQSKWDLSHDCKVNPFSESEQGQALLAERRSIQNRIEKEFVRLVVTGLFSVVKPERIAYLKMENVDLSQIKDNNKPKSLYMTAHDVWGMDDGKLSSKKNKVEFITEVQYDLRAITSTEYWNVISVTHDGDTVVVNAEPTDKWREESARYWIDAYTHRALHFSSVKHLIEELCIKRCIHFVLIDPAHTSQLCHVCRSVEHINFKGSDTEKLSREECLSKHVNFRYGRTFVCGNPECYMCGKEQDSDDNGAWNVLLPQIVIKRKRVKKSKS

SEQ ID NO: 4 - protein, 827 aa, unidentified
  REGION 1..827: note = uncultivated organism
  REGION 1..827: note = MG122-4 Effector
MKKVKIFSSKSVTRTTTGPDNNPLYRKLTRKFPFAFQDVEDAKWVIRISSGVKDSMNRVLGILRDHFFDPIAATVGGEERVITLFDLFDPGCKLETPFVVDGKTTNKVLSGNYTVREIFNVKCPLVPVDRDALVAEFCRQVTSGEHDSLPYLTVFANEGIFAKVCTPVRVAVNRKLNRVKVANARLRDRTWPTCCREAASAHTTNDTEAAVLSRQVEVVGVQIHALNNGGVPVIKKWPRACKVTVGKTSIPVNEPELDEDFMRVYREAVELFKAAFKKAFPVIRRGVLLEGASISIDREGRDTSLYYDTPGVLSGIPSRPGTQGIRVRFRTVSGRGTGYYGEDILDRITGNPKFGMHIPEDATPEGLCYGEGGLARSGCNAFGHVFTEIPLVETARVPDAENPEEFLDIPEEGVGIDVNVGDFLMNTTVALSETTGAVDWCEAIAAFRAARPDWYPFTRAAHRVVREMHKLADEASRDGRLVRVGLLVGLRDGKPADAAHGWKPAPDPLCTLFHWMIRRTGNDGTPFYTREQLAHIGHTRDYRSLVRKELANYLHYFYEQSKWELKHDPKVEPFCETDAGRKLLKERWAIGDLVEKETARLITTGLLDIAADDRIQYLRMEDVDLSAIRQDRSAKSLFKTAKDEWGMCDGKLSAKNGKVEFVTGCGFNLAAVKDTEYWHVESVTREGDTVKVQASPTAKRVDEEARYWTDYHTKKTLHLSSLKKMVEELCIKRGIHFAVMDPTCTSKICHVCRSVGHINHRGSDTEKFGPEECMRLHLNYRRGTTFICGNPECGMCGVIQDAGDNAAINVRMPNIFVKTKRVSKKAA

SEQ ID NO: 5 - protein, 619 aa, unidentified
  REGION 1..619: note = uncultivated organism
  REGION 1..619: note = MG122-5 Effector
MNTDIETNANIEINKNKETKDMKIQEQKEIDTANVVDNTIKKFRENLRMSYSADSHKLTYYDGMLNQPVLAYNKEKGGFYFLAALSEHEAKDCQLDMTSQRLFKNYFTSALSDNYRQTNKHTLPDGTFKIVGIDLGLRQPFVGVVYEVRVELGQIVSHKFLRYIKANASENAGIKVEILHNLFDQIKALKSLLYETNKYFVTKKEEEIPDSYRPSFDRAAIAYGDILGIDMDMYRAYVHNFNNINELRHDKNWIISTLIKNLNSSPQKKCGKCGNIDNPTSEQCSKCGEPSRIVEKKCGKCGNIIQMEFMGDMIYRCPKCNNSITGIKDLRTEYTRIKKGEKKSYCKKNNYTDWTIFLKKKIILEYISLLRSFSTIGLKTSDTIGIRKNFAKTLYRWHKGLVRKCYLLSADIINVVRKEGAKVLFLEDLDIKTSIADNREENKLKALWGWGEIKKHLGHQARKHKIAVVAVNPHLTSLVHYETGLKCIETEGRMVTTIKDGKKICIDRDENAAHNIAIRGLYRHANLREFSVDQIDENVYRLNYSQYDGKQKKGALFHHLRTLKCAFVRDNDGVLIKKDLTPEEEKKGEINKKRTYIILHGDKWFLRHEIDEILERELR

SEQ ID NO: 6 - protein, 529 aa, unidentified
  REGION 1..529: note = uncultivated organism
  REGION 1..529: note = MG120-1 Effector
MSEHFITSIPLYVNEYAYSVLTKRFNVAHHIYNCFLGECKKRRDKYLKSDLYKEAGTKYKEFAAKKVGDKKEINELFKSAITESGFYLRSNSHGRDNSLEQFAASTIQRTWLGDHIDSHCRNKLMERAFKAAQRSLKKELNKRTGGYERPKLRFKRFGRDYICSLEGKNSISPLKFEFKNEIYQVVWRGLSLPVYFDENDDTIKHAINNIENISYIRLKRKPVRGKWQYYADLIIDGKRLLKEKNKLGEGVVGGDLGPGTLACVSNEFVILRSTASNVLESNAYLKLEKQRRIIQRQIDRQRREANPDNFNENGTIKKGSKWIVSKRQKHNENQLADLQRRIKEMRRDNHGAIANELRSHGDEFRLDKDSYKGWQRSRYGSSLLKHAPGMLVELIRDKFENTGGKIDEINTYDSKSSQTCPNCGDISKKQLDERIHDCKCGFIMQRDIVSAIVNKFWNTEKKTLDVDKSKRFIKDKRSVLQAGIEKILQSVREGHLLPSTFGTIPELERIACQFDDQISSDACCLQKTT

SEQ ID NO: 7 - protein, 477 aa, unidentified
  REGION 1..477: note = uncultivated organism
  REGION 1..477: note = MG120-2 Effector
MKSKTSTYTVEFELITTFQDRYIINKKMKVAKSIYNACLNECLKRLKAVKADKEYHTLVKTESSNQRNSRLKEIERYYGYSEYDLHSWVNQVKYHFEGHLGINEVQKLATRAFKAVEKFHYGKAKKVYFKSFKDDMSVENKSNKTGLRIKDLKIIWGNLSIPFKVKKNDKYAQLALLDKTKYVRVLKREIRGKERFFVQLIQEGLPPKKHQISTVENRVGIDIGTSTVAVVSDNEVFMNELATIKTPYKKLRKLDRAMDRSRRSTNPQNYNDNGTVKKGYKKWNYSNRYLKLKYKRKELFRKIASIRKQNHEQTANFILSLGLDVRVETMRFQSLQKRAKKTTRNKKNGKINKKKRFGKSIANYAPAMLLEIINRKLKYQNLDLKKIDTYSVKASQLDHTTGECTKKFLSNRWNNIDDKLIQRDLYSAFLIQNTTNTLDVVDIDLCNLKFDNFVKLHDIEIQRLKNLNNKKLNWFVA

SEQ ID NO: 8 - protein, 477 aa, unidentified
  REGION 1..477: note = uncultivated organism
  REGION 1..477: note = MG120-3 Effector
MKSKTSTYTVEFELITTFQDRYIINKKMKVAKSIYNACLNECLKRLKAVKADKEYHTLVKTESSNQRNSRLKEIERYYGYSEYDLHSWVNQVKYHFEGHLGINEVQKLATRAFKAVEKFHYGKAKKVYFKSFKDDMSVENKSNKTGLRIKDLKIIWGNLSIPFKVKKNDKYAQLALLDKTKYVRVLKREIRGKERFFVQLIQEGLPPKKHQVSTAENRVGIDIGTSTVAVVSDNEVFMNELATIKTPYKKLRKLDRAMDRSRRSTNPQNYNDNGTVKKGYKKWNYSNRYLKLKYKRKELFRKIASIRKQNHEQTANFILSLGLDVRVETMRFQSLQKRAKKTTRNKKNGKINKKKRFGKSIANYAPAMLLEIINRKLKYQNLDLKKIDTYSVKASQLDHTTGECTKKFLSNRWNNIDDKLIQRDLYSAFLIQNTTNTLDVVDIDLCNLKFDNFVKLHDIEIQRLKNLNNKKLNWFVA

SEQ ID NO: 9 - protein, 477 aa, unidentified
  REGION 1..477: note = uncultivated organism
  REGION 1..477: note = MG120-4 Effector
MKSKTSTYTVEFELITTFQDRYIINKKMKVAKSIYNACLNECLKRLKAVKADKEYHTLVKTESSKQRNSRLKEIERYYGYSEYDLHSWVNQVKYHFEGHLGINEVQKLATRAFKAVEKFHYGKAKKVYFKSFKDDMSVENKSNKTGLRIKDLKIIWGNLSIPFKVKKNDKYAQLALLDKTKYVRVLKREIRGKERFFVQLIQEGLPPKKHQISTVENRVGIDIGTSTVAVVSDNEVFMNELATIKTPYKKLRKLDRAMDRSRRSTNPQNYNDNGTVKKGYKKWNYSNRYLKLKYKRKELFRKIASIRKQNHEQTANFILSLGLDVRVETMRFQSLQKRAKKTTRNKKNGKINKKKRFGKSIANYAPAMLLEIINRKLKYQNLDLKKIDTYSVKASQLDHTTGECTKKFLSNRWNNIDDKLIQRDLYSAFLIQNTTKTLDVVDIDLCNLKFDNFVKLHDIEIQRLKNLNNKKLNWFVA

SEQ ID NO: 10 - protein, 475 aa, unidentified
  REGION 1..475: note = uncultivated organism
  REGION 1..475: note = MG120-5 Effector
MVNKTSTYTVEFELITTFQHKYIINKKMKVAKSIYNACLNECLKRLKAVKANKEYHALVKTQSSKERNQCLKEIELSYKYSEYDLHSWVTQIKQYFEGHLGINEVQKLATRAFNTVEKLHYGKAKNIYFKSFTDNMSVENKSNKTGLRIKNSKIIWGKLSIPFKIKKNDKYAQLALLDKTKYVRVLKREIRGKERFFVQLIQEGLPPKKHQTSTVENRIGMDIGTSTISIVSDNKVSIKELANIKTPYKKIRRINRAMARSKYFANPQNFNDNGKKTLKKWNYSNKYLKLKSKRKELFRKLSIIRKQTHEETANEILSLGLDIRVETMHFHSLQRRAKKITYNQNNGKINKKKRFGKSISNYAPSMLLTIINRKLKYQNCELKQIDTYSVKASQFNHTTGEFTKKLLSDRWKNIDNKLIQRDLYSAFLIQNTTDDLKTIDIDLCNLKFDNFVKLHDIEIQNIKKSQNKLLHWFVA

SEQ ID NO: 11 - protein, 517 aa, unidentified
  REGION 1..517: note = uncultivated organism
  REGION 1..517: note = MG120-6 Effector
MIRKGHLVRLQTPSFVVSVKLLLPEQVQNHLEKSFHIANSAYNEALSFGLRRFDAMKQNSSYQELLELRRELLAKSDKEQKTKEFKAQKKENDNKLAEIRKDYSLTEFGLSQHLSQQRRKPDSAYQHLNSGELQVIASHVMKALEKVLFYQIEPHKVRFRSKYDADMSFRNRVNSTGTRLVESNKTSIAYRLYIHKASTFVDIPIKAFNTYQQMSLLRSERIKYVQIIRKTIRGKKVYYLQIVCEGTPPAKVNRGQGVVGIDPGISTVAYAAPNKVALVDLVPRNITQKEKFLKALDRRIERSRRVNNPTCFNEDGTIKKGSHFKHLSNRALRLVNRRKKSYRSLSEERKKLQGQLINQLVSNASVIKMEELNVKGLQKRAKDLQINPKTNRPFSKKRFGKSIFRAAPSAFREALKTRAVQLGIEIIMVSPKNVKPSQYNHILQTFEKKPLSTRIFDLSPEWTGLQRDLYSAFLIGHIENNQYQQTELEQDFPTFYQQMRDFLQQTPQTSRLAWYFS

SEQ ID NO: 12 - protein, 528 aa, unidentified
  REGION 1..528: note = uncultivated organism
  REGION 1..528: note = MG120-7 Effector
MPPCTSTSRSKSTSSTPSFVCELLLRVSPAQEKALLARLEAARQLYNACLGEALKRVGLEQQSKLWQKAWKAGDKELRRALIRKACLKYGFTDAALQHYAVKIRRSCWIGDHLDVHVAQKLGTRAFRAAQRVLFGQARKVRFKGKNQMDTVEGKSNGAGIRWRNGRMLWRGLVLPAIIDQEDPVIRHALSCRVKYVRLVRRKINGRNRFYTQLICEGTPYQKPENALAEGTVGIDAGPSIIARVNSKEARLDRFCEELAKKEAEIRRLQRRLDRSRRANNPENYNPDGTVRKGPKQWKKTNTYLKTEARLAELWRRLAAHRRSLIGKMVNETLRMGNVFKFEKLSYRALQRMFGKSVNIRAPGMFFQELARKAESAGGEVLEFPTRLKVKGTETCVGLSSYCVCGEHRKKPLSERWHICSCGVRCQRDLYSAYLACFVKQIGQNDFELDAGTAARQWPGAEPLLQAASGRIPNPQMSGSSGQPRTGTEGVAAEGGTAKTEARDVVAPPPGGGESPGEVAVVPLRTPGL

SEQ ID NO: 13 - protein, 425 aa, unidentified
  REGION 1..425: note = uncultivated organism
  REGION 1..425: note = MG120-8 Effector
MAKKENSKSFILTFETGLTKRDRDLCEKKLRIGRSMYNAFLGEALKRYNGYQKDDRYLTAVKLYRDAKEHNDKEEMKALSEVMNQVKSEYRWGGKYCLNDMRKEQSRHFKGAIGSMIAVSLSHRAYAAVEKMEKNTDVTKVSFVRQSDDFSIEGSTNDNGIIYRNGRIYFDISKDVWVDIKPARKNDDWYYDEALKSRVKFCRLLSRTIRGKKRYYVQLCMEGTPPNRREYGDKRIEILDVKISHAELKKEGEKPQIIELAPGCKNPEEKIAELDRKMDASRRATNPDNYNEDGTTKNGPLKWSYSNNYKKLRAKRAEIYRSMKERRKVSHEELTNRILAEGTDITIKHLSYKEQQRRSKKDTEINPDTGRPYSKARQGKNIANRAPSEFVSILSRKLNYIDKAFADESKENRKDEKNENKIIRI

SEQ ID NO: 14 - protein, 670 aa, unidentified
  REGION 1..670: note = uncultivated organism
  REGION 1..670: note = MG120-9 Effector
MPKPEGGTHVCEIPLQRSAHKEKILGVRFGCGYQAFNAALDEALRRIKLMRQSKDWQKARKLPKGNSDGSKEQKAQAKARGEAFNTACLKFGFTEFSLLEIWTSKPMSPESRLQGALKRKQRRLRELLARQSEIEALPETDKDRTKLLKENEQARSKAVSDLTLLAITPLESKPVGKLGGSWLVKHLGVHVCQAATSKAWVAIQEYSFGKKGKPKFRSKYDPPMSVDGKSGASLRWNKGRVEWGGLILEPLIKRDQHGRPVDPVIAYSLHKKIKYVRIVRKASLRGYDYAVQLVCDGPSYNYQREVGTATVGLDLGPSGIAIVAHEGSTVVKAELCKLAPEIIRNSEKIRRLQRKLDRQLRANNLDCYRPNGTFIKGSKAHNRSNRQLGTKLQLSLIQTKQAAHRKSLHGRRVNELVSLGVNATTEKLNYKVWQGHWGRSIRDRAPGLLIEMYRKKCERYGGKLGECSTKTTKMSRTCHCRTVKFKTLSQRWHSCTCGVGPVQRDIYSAFLASCVKQDVVRGDQLDVESLKKLWDNGADKLLKAASSEAVKAGLVRPYRKCEKLTPHGVDSPSNGSEHSSEDPDSKLEGISPEHVAEDEIQDVVPTACGKGEPGGVFEPNRNATDLSTHRERRGGETRQPQSSQQLRLFEGDSKQSLSQSTTQVQEIVEL

SEQ ID NO: 15 - protein, 786 aa, unidentified
  REGION 1..786: note = uncultivated organism
  REGION 1..786: note = MG118-1 Effector
MKQKDDFTIIDRLLLSNFPLTSYYPAKAKDKREALRVFERCGEQASIDYLKTRIGAKSPIKQLASKYLPNAKFLNSTLKVAARKLKEQGEQKALEYLQANVSEDSYDLKSPVKCEIVATSKKLEDFAIYNASRDIQCYIYNLSKDEFLSCVPGTSKESHQKWFSAHSALKQYETLGVQCLNLIFSNAISTYKGVEKKVAEANAKKIKKIEERKARGLVVEEFEPKKVYGEDNKLLEVPGINPVIYCYQQISPKVYVSGQVDWAANSSELQIYRGDIEGYKQYDRKPDDPIVTLVTQKSRLEISDGSPGHIPEKDKDTLTLNKHRRLHKYWSSEVEKSKNAILVVIRMFDGWVLVDLRGLLRQCRYRKLIAPNQKITVKELLSMVTGDPVVDVERKVCRFIFKPEIIRVKKKNTIKASKKVAENLAKQQTTLLSIDLGVTNVVAASVYSVVDGKMERLMEPFALTDEQKDNFTRCDEKRDRINANIRKEAIAFLTLEEQAEIRKLDTFGPEEAKIAICKKLGIDDVSSVPWDSMGANTTILSDIYLANGGDLEKVYFTPKPKKNGKGKKVPDPIKSKDFYWSRKYRPRLSTETREHLNEKIWELQRANKDYQKLAIRKKELSRQVVNYILAKVENRTQCGKVIIALEDIDVKMFHRKDARISGWDGFFVAKSSKQWLIKEMHKAFSDLSQNRGYIVIEVRPAYTSQTCPKCGYCHRDNRDKNNREKFSCKKCGFTAHADFDVATYNIAKVAVTGERLKGPEKKNKKISSKEVSSGYMLQELPSLLEI

SEQ ID NO: 16 - protein, 640 aa, unidentified
  REGION 1..640: note = uncultivated organism
  REGION 1..640: note = MG90-3 Effector
MSFGPSKIVFKMGAKLPLDDGPAVVDRIISGAHKYYNKLIEFGRRQLLEKEELRREFLPGLAEAADRIEEARQAHEALADDIRRRNQEARRRQATPEDLALRASSANRLAEARRTRKEILARVKDDLRYLQIQEQRHLRDNRTLAALYNEADCNWGTRLQTGRAFESAMKIMNPATNKMMERGVDNPPKFKRWTGDGLVTVQTQNGLSLEDLLAGKNGQFRLEKLPPKNGNLHRALAWLRVGSDGDGEAIWLKVPVQWREDFDRLPPGSMIKQASLVRRRGPHRRFASPDNVLGVWRPYDRFELQMTIETSGIRDTSKLATAGLCGVDLGWRLKEDGSLRVAYWVGDDGQEGEVLVPADRWLICNELEIICKDNFNQHVAILSRWLGAQASLPETLTEKTSTLLSWKSIGRLCRLVDTWQPVPGDEDLIKVLRAWRVQEAHLLDYERGRARMMANWRKDHYRQLAAFLAKQYRTISVEDCDWRKLNKLPPVESNDPVNLTARRNIRIAAVGQLRDCLRQCGANTILSETENTTKECHVCHSIEEFDHRKLFHTCSQCGNTWDQDRNAALNLIARAEKALREMAEIETETVPVEGVGAASSVLGSRGSQDQASEEVVAVPPKKGGRWQRRKAKSSRRAAAT

SEQ ID NO: 17 - protein, 611 aa, unidentified
  REGION 1..611: note = uncultivated organism
  REGION 1..611: note = MG90-5 Effector
MPFNNKASESGLDTRVYAYGCNLKKDVADVLMIQLRLANKYYNRLVDCKKDQLHAERETLYKLSPALKQLAQEIEKKDDEKGELVRQAKRMNVAARASGAADDPLRAELAGVEAELRDLYDREKALAKPLYRQPEFVDERARLQAVFEDACKQVRKESRTPNEHGDVLGWGTAGAWYDAFKAACKAPMVLFKRAKKSGDTRLYVAACKLLMRGPKYRRFSGEGRVGVQITGGGVPVPDVLDGKSTFVQIDPADPSTSSRPKRRNGRIRIGSGSTTRVPLWADFRFRMHKGHALPDDGIVKRAYLIAKKIGPRTKWEFQVSVAGRPKDWPDRNVTDGVVAVDVGWRDMGSHVRVACWLGDDGRYGEITIPAERVVRYQKVKDLQSIRDKNFDAIQAALMAWKKAAKDDVPEWFSERFESLHSWSSQARLVSATRYWEQNRFDGDESMFEEVRVWWRQDTHLYDWQESQRKGEERWRRDHYRKIAAELSRRYRKAVVEDVDWGVFQRAQDPLKAKEINEERRAAREERRRIAGVSYLIGDLKNRLVEVFEAPPEWTTQECHHCGCIDHFDAENQLRHTCSGCGEEWDQDYNACYNLLRLEREAASGSGVEDGQ

SEQ ID NO: 18 - protein, 762 aa, unidentified
  REGION 1..762: note = uncultivated organism
  REGION 1..762: note = MG90-6 Effector
MKLKTSSIPTQIWSFGTPPPKFWSPGPSTSEERPRYRRLGEPPIPLAQFTGEPFVREVLFRGNRFYNDRIALERTRYAEYVRIRRSFSPALAACEDAYVAADVAAQKAVQDIKDARAKQYRQSKKKSDEVPAALIEALRVARAQQKETKTQLKRARAHFEAQMKPTRLEYDTRCQTRKLALASKSGITSPHYKGRVNREVLREMVAETQWPQAWKDLQSMEAAKVRQQKVVRKQRPVGAGIGGLIEKATNQAVKMSKFGPPGFSRFECDGRVGGQINPPCSMRDLFTGKNSKIRLRPQAPKLKRKHNLNHGKWKFDNQFFILALNIGTEVRNQAKWIEVPVRIHRPLPPDGKITQAWLLVRRQGPKLRYSVQLSIESESFLLPKKMNNGRERPIGVGRVAVNFGWRMVPGGVRAAYWTSETGERDELVLGSAEQEIFYRLTHPDVLRKHADEHFTEVRRGVRRSLRSGGGARLSKTPTVAETGNRLTHWDRWARKDEDSQRRQFTSLLWQWAKFRIGKARLDALWHEWKGYRLAHTEARPDGLDLFAHRNVASEWAKEHGLTKASERLAWWCHLWVRKEIHLAKYEADLRFGSHNQRDAIYRTLALKLSTQFGELITDNTKFSDLARREKVGDEGTEAWEKARQNRQAVGAGRFRAIFRDVFGQRGIVVSAVDNSVICSRCRQKMNVVAGESEVQCKQCGPIDVDHNNCLNMLRRADDGERITRDQKEDAPPEQPSTPGPENEQSTGPADSYTHDSLWDAAE

SEQ ID NO: 19 - protein, 585 aa, unidentified
  REGION 1..585: note = uncultivated organism
  REGION 1..585: note = MG90-7 Effector
MSGIRILNWCYGAYSPEDRAPVFEQLRLAHRFRNRLCELEINCRNEIEAILCERSEEYVSACRNVSDCEGQIEEIYKAVKLRGQQERKKPKVSDDEAEAIKVLKSQRLKLWELRRKARTAAFARSDVEYSISAIRNRHNDLRKQARVADGLYWGTAATVIDSAKSFSSGAPPRFTAYRGQGTLSVQFINGMPAERLLSGEDRRARLIWAERNRYLLWMRIGSQDDRSPTWAKIPVVMHRPFPTDAILKWLHLDLRTCASDEHWTARFALQTASTVRFPIRPLVAVHCGWRTLDDGSVRVAVWRDTEGHTGDIRLTNKDRLSLEIPEGLQSVRDKHFNEAISFLKQQFKSADIVPEWFSVRTTTMLHWKKQSRLAGLVCYWRDHRFCGDEIAFDTLESWRKRDKHLWLWQQNQRRKWRIRRNQLYRSFAKDLAAKYGRVSVMAAEWAKMRKVPEVEEVERQSTRLRRLSAGAAVATLEGYIKEAFGRDCVVAIDIKNVSRQCASCGQLQTEFTKRVGDCDKCGTRRDIDDNGARNQIARAGLTLKSLEPLADVMRQRLTAATSKPAPSERHQRRLAARKRLAEAKQ

SEQ ID NO: 20 - protein, 589 aa, unidentified
  REGION 1..589: note = uncultivated organism
  REGION 1..589: note = MG90-8 Effector
MFGHTSAPSRIFRFQARLPTAGLEVIEEQLTLAHRYRNALVALERRRRAMVHHALVQAMPTGVAWEATVAMLEAMLESHRDVLARQHAKKRRREISAEAKAIAAILKQALREARGHHRALRRLAAGKGLDADDHTHQMLHHCQLTAQDASEALHQVDEAIHAERKRLRANCGLYWGNYLAVEQGMQAARSGPPPRFRRYDRQGKLAVQIQHGMSPEELHSLQDPRLQLEAPKVDDLGRIYRRHRRVLCRFRIGSVGREPLWAELPVIMTRPLPYDSRVKWAYLKRERVATHLYWYLELVLSRDKGWPKPDAAQTGIVGIDPGYRRRPHGLRVMYWLGDDHQGSELVLPEEWLQKWSWVFELAALRKRRMNAIVADLVRWVKDHGRPAWWPEDTRTLARWCSPAQLAALTLQWRREHADEAPDMLARLEAWRKMDKRQYEEQEHLRRHLIDARDDLYRKTALALRRRYQTAVIEDTDWEALARREEAEEVRRLPAIVGRYRQIAAPSRLTQILREAFAEVLDAPSGYSTQRCSICGRLMGITRRDGTYVPPDNASLEIRCPWDHTLHDQDANAARNFRDAGPALRKRAAR

SEQ ID NO: 21 - protein, 625 aa, unidentified
  REGION 1..625: note = uncultivated organism
  REGION 1..625: note = MG90-16 Effector
MVQRSTFTGHNCVYVYGTPSKVDIPDYAMEQLRLANRFWNGLVEIQHAHEQAVAELWASHPELAKLNDEIAQLVEWGRELKEKADAARVKGRRRYITDADLRNELKQQREQLIAAKKSYRESKSLFYNQLKPQFTTLKEKRVADIKALRQAFASQGLYWGTYNAVSQSFDTAAKRVSSLRASGRSSALRFHRFDGSGTWTVQLQQEAGDPPRTWAALVGGQSKWKNDVRIAPEELYEGGMRKHPRTLLSIRCGRQGKDALYLTIPITVHRPIPPEAEVTQIQVSRKRVGTHYELSAAFSLRMPTPAKTKNPGVVGINFGWRSKRSDELLRTAVWYGIGRDTQLELREWMTEWVTQERLAKGKMVGEIRVPDRWRRVMARMHEIRSQRDLNLNAIRGEVVKWLTTHKRSANQLASAWAQAHPNASKPLWNKPSDVGLWRSAGRLAALCLFWRTHRQSGDETIFAVFEAWRKQDKHLYDWEAYNTRQLIVQRREVYRCVAEQLTSIYGHVVVDDANFQTLTMKETRLEEADSEQADLARSQARDASPGLFREIVKHTASRLGTVLTVEPANGITQVHSVCNTWVDADAAQKIMLWCPVCETSFDQDANAAMNLVRLATGQVAQLVKS

SEQ ID NO: 22 - protein, 625 aa, unidentified
  REGION 1..625: note = uncultivated organism
  REGION 1..625: note = MG90-17 Effector
MVQRSTFTGHNCVYVYGTPSKVDIPDYAMEQLRLANRFWNGLVEIQHAHEQAVAELWASHPELAKLNDEIAQLVEWGRELKEKADAARVKGRRRYITDADLRNELKQQREQLIAAKKSYRESKSLFYNQLKPQFTTLKEKRVADIKALRQAFASQGLYWGTYNAVSQSFDVAAKRVSSLRSSGRSSALRFHRFEGSGTWTVQLQQEAGDPPRTWAALVGGQSKWKNDVRIAPEELDEGGMRKHPRTLLSIRCGRQGKDALYLTIPITVHRPIPPEAEVTQIQVSRKRVGTHYELSAAFSLRMPTPAKTKNPGVVGINFGWRSKRSDELLRTAVWYGIGRDTQLELREWMTEWVTQERLAKGKMVGEIRVPDRWRRVMARMHEIRSQRDLNLNAIRGEVVKWLTTHKRSANQLASAWAQAHPNASKPLWNKPSDVGLWRSAGRLAALCLFWRTHRQSGDETIFAVFEAWRKQDKHLYDWEAYNTRQLIVQRREVYRCVAEQLTSIYGHVVVDDANFQTLTMKETRLEEADSEQADLARSQARDASPGLFREIVKHTASRLGTVLTVEPANGITQVHSVCNTWVDADAAQKIMLWCPVCETSFDQDANAAMNLVRLATGQVAQLVKS

SEQ ID NO: 23 - protein, 640 aa, unidentified; identical to SEQ ID NO: 16
  REGION 1..640: note = uncultivated organism
  REGION 1..640: note = MG90-18 Effector

SEQ ID NO: 24 - protein, 634 aa, unidentified
  REGION 1..634: note = uncultivated organism
  REGION 1..634: note = MG90-19 Effector
MAFGNKSKPTRKYNYGCRGFLSGPREINPDGTKALRQRFDGHDLAVTQMKLAHQFQNKLVEIERARREAIDPILVRHFPALTTLHNAAQEQQERLQELRDELSQSNSQNRQLMSDPELVEQINAQKRVVSQAWEFYSDQRDAAFADPKVKADLKDNDKSFEQRKADARNAAVEGGLYWATSLQVVGRVKRTGPPPKFKSWRGEEVISVQFQRKPDKTSPKEPVLDSKGNPKIHPRSKKPTFAHVGGSSLRTCDVFTPNTNCWIERTYQPPLTAPHPKYVVIHFRVSSDEKGKPVMASIPAVMSRPLPEDGEVKWVHLSRRKIGTHYRWDVQFDIARDAWTYHPAGQDRAQEGTVAVALGWRLIDGEIRVTEWVGDDGVTGTVRIPKELVEGWSYLDTLQSIRDTLFEAERAELVDWFVNYPNPLPEEWTERAQTLIQWRSADRFMWLIWWWKDHRIPGDEEIFQRMWGRIQLNPTTGRNQYTGGRLQSKHLCDWRAHKRDKIKNWRKDFFRKVAIDLSYQYKDVVIAEIDWNKLAENPEVENGNDIVNKRYRALSSCAQLRDEITRYMNEVTESARNIVTTCCQCGESCDHPKSGRWIRCESCGGEKRDRAVNAATNLLNRALGASGAKVPARV

SEQ ID NO: 25 - protein, 643 aa, unidentified
  REGION 1..643: note = uncultivated organism
  REGION 1..643: note = MG90-20 Effector
METRILEFRAALPLHRYPLHKAGLLGADLAERNSEKPCFLEACYEYRRQLVNIELYRRQARRAVLLKLYPRLVALSDQIKAMTVELTRLRGQLSSARARVRARITLPAVEKQIKLQRAALAKLRTEYNKAHERATALSSLEGAQASLKSNRNKNKPAKQETLHESIAKYQEIADSYTPSELDSSRKLADEYYAIAKDVDARKSALYGSSDLHWGTKNAISEGVEASRSGPPPQQRRGEGEVMFSVQLQAARRGGETRSANWQALLTDNAWSSQVRLRAIPAAEVAGFSKSRPGQAIRNGVKSQHQHMLQIRVTADSEKGWVEMPFHFRPDRWPADAKILYVRIRRRKLRARYQWLVQFVVTSEQFSRQVGTGEVGINLGSRLLKNGLRAAYAVDDTGHSETLELPHEDFVRHWRYTEELQAIRDREFTAMLTSLRDWLKRHKKLLPGWLREEAKTLHLQKAKRVARLVMRWRDNRFDGDEKIFADVNYWRSRKNGHLLEWQNSEMGRTNGRRKSLYQKWAADLRKRYGTVYIGKINYRELREVEEPEDEAGSSEVRKLAARAAIGSLKVILQMSGMKVVERDPSYTTQLCHVCGHVNDFDAASRLEVVCESCASCYDQDYNAAHNLLTGRRPKSAGKELQAVV

SEQ ID NO: 26 - protein, 705 aa, unidentified
  REGION 1..705: note = uncultivated organism
  REGION 1..705: note = MG90-21 Effector
MMGAQVRRGGVVTTQSKTKKRGGRKKRSKEELREAQRARMRKRNKAKKKAKEKDKEKGIKYKDTGVRVYKYPANRTLPLGFENEHRFLGQLAEQGDRVNKDLISAEMRRRAHEEGLLLEGVEFVDDHFFHSQRMRNDTVEIDHSMRDLDMKFMLKTELKDEILEADAKYAAIKKALHAVHEEQRKRNSKNRDDTLTPEERAEISALHKKLKNAWEDRQKICKEARELDSYDKKAFDDAKSDAAKEGRERFPELHWNTRNTRHEALRQHRKGPPPKFKSWDGSGIIGGQTYQSREKQAPEVIFSGDLGQGHARIVRYPTGVWVAGRDRAAKRGDAILYLRVRSEGKKPVWARIPFFMKREIPEGVRITHAKVVRKKEGRRLKWSVQLSLADVGPRPDRAKEGTVAIDVGWRYDNGSIVVATFSGSDGREGKVALPCWWLREAKQVENLQSLVKKDFNEAVDNLIAYFKVEGRGCIPKGLRKEFKNLHQFRSPKKLSKLVKKWRDQDEDSCLDWLWHWRGRQLHLGPDWRDPLRGQLQGSRRDLYRRFARDMGRRYKNCVIEEMNLAHMQERNKAGDNKKQDRINKNKRHAALSVLKKSLQNVFFEDIDTLNPAYTSQTCWSCGKRNRSLGSAQVFTCSCGETWNRDVNASRNLLAASGAKVNWDRPALEPDEVWTYSEKPNKRRRKTIAKKRALEMKRKNEGGEEA

SEQ ID NO: 27 - protein, 698 aa, unidentified
  REGION 1..698: note = uncultivated organism
  REGION 1..698: note = MG90-22 Effector
MEPVKKSNARKTSTSETKVYSYGAFLPKESEQIKLINDQLYFAHKYRNKLVEIERKRRNRFRNLRKLMSPELRQLENDLLLTEEKIIELRKSFGGRAENSLDPKFPKKNRQLTPQAVEIKDLKLKKKDLSAKIKSIHTQLNLDYFKEADSKFSLLKKERLEQKAKELNKDSLGPNDVNRHNVVNNLYKEMIDGGNRFWAIKAKISKSAEASNKRARSNCKCSSGTYVSIEEAAKQSFSNSKFEPKFKSFDGSGKIGMQLTQNKGLSIKDALSGSSPVLKIDLHPEVYLRQNKKKNKVLATARIKLFGTEKTGKFVDIPFIMHRQMPEDATIKWVFLVVSKIGYRSIYNIQFTIESNSFTQPSAIRPDDVAINLGWKVNDQTDDITVATSFDGKNYNELILPSKMRANIVYKETLISHADKHFDSVKKDVSKWLKNSNLDECITKYFTNLPQWRSHKKLLFVSKELAKVFLPDNTWYDLWKKWKTHCKENKPWKNCSDKDDLFTTLDNTIQWCKDNSIDDPNVQMAFYLKTWAEKEIHLINWARGIESKLRKHRKEIYRCFAKKLSSTYGKVIVENWDKSKTAETPDVENDNRTKQEENANAVRQFVGVSVLTDALKQKFGKDFCEENAKNISKEHFKCGGELINQKELSDVHCKKCNKSVNVNYNAAAHLFDRHGERSGAVKLPGTARKTRKSPELLA

SEQ ID NO: 28 - protein, 685 aa, unidentified
  REGION 1..685: note = uncultivated organism
  REGION 1..685: note = MG90-23 Effector
MLVYKWGIGPLPLEARQIIDREVRAAHRYRNRLVEIERSRRAAYRDLRNSLSPELAHLHAAYAAADRAVVEGRRLLSGVPRAERARHPVAAEVRRLSAARSGAWKAYAEARDRTTADVFGAADQKYREAKQSVTAAVGFACAVARFARGADPGPIGPHVLAAITAAVRERALADPAVGEPWKAKTRSQMEHEALAKRARAECGCAVGTYLAVEAAAEKSFAECAGDPPFSRLECERVGLQVRGGGLSADDVVGAAVGQVRVEFPADMTARANGTRYAVVHLHLSGRGDQATWLHLPVVWHRDMAPEARVRWAYVVARRVGLVWHYELQLTCDSVDRARAQSAGARGTIALNLGWRALKNGDLRVATPWSGSASRAEDRLVLPRSFREGSDLADRLLSYADEHFLAVRDALAGWFKGGERSLFSPEQVAAWSLDTVHAWRSHGRLARVALDLRRDWLEARGVDVPALWKAWRLERSPKLDLFGPLDEIKAWLAGRGVVSADQVLAVYLDWWRAKDRHLVNWARNNDLRLRRSRRDRYRCYARDLAARYERVVIEQWNKSETAETPDPEADTRTEQEVRGNSNRVLACVSELVDALEAAFGEANVHRAPSERITVEHHGCGGESSDPLPQIPVTCFACGQVYDQDLNAAKHLYDRHSGEPSGGVNVGGGARGAKKSRRVEGFGRAAE

SEQ ID NO: 29 - protein, 680 aa, unidentified
  REGION 1..680: note = uncultivated organism
  REGION 1..680: note = MG90-24 Effector
MTTTKLYSYGAHLAHCPAKVGALLRKSVEYYNALVALENLRRAMFLGIEEDASSDVAEAMKVVNEADAAVELAFGAISEHRKTTRARSIPVDLRSAKEAAIERKKAAKITLREAKAKHRAQFDKPNDEFLRRKTAATSDASLSELRELSGEDLRKKIASIREKLGPIAPRTAEKLNAEILGTMVGESEWSEHWKVVQFLERVIADHEKEARRISGLPHGTYSLVDDAFEAAKRTQKVGDLRRKWIQREGDLPVDDSGRVGVQLPSGLSIADLCAGNHARFKLCVETPEERAVKGLGDRSARWERRNIRAIATIAIDSSDSGWEYLDIPISYHRSLPPNATVKWVWLFVQRHGMRLHYDLQITLVIPDDVDLGQSRRAEVCTINPGWRHFDDGSIRVAYVVGSDGHWEDLRLPATEGKRWSVSEQMKFSDSLRSASDSLFNEARKVLVGMLPRLPESIQLQCTFIAQWKDHTKLQRAVIATLAESWTSNSCQNAFEKWVAQRKARGLDLMPTLDEAVEFSPTDPERWYLYCWLRKDRHLYQMAIDISRKAARDRNQMFSIWAKRLSERYQRVALAEGGLKKLAKKTDEVASSQLRAKSRVATGTFVAKVKEKFGKPNVIADKGVNASRTCVKCGHLHEEIDLSRDHMYTCSQCGYEQDQDETNCRNRLTWLRDRRGAEKTE

SEQ ID NO: 30 - protein, 433 aa, unidentified
  REGION 1..433: note = uncultivated organism
  REGION 1..433: note = MG119-1 Effector
MVYTTKFYLIYDDDNNPIDYAAFCKEMFGIQRDIRTFKNLASSEYFSYMMRRTRWKDEHGAYPTDEELLGDKIGNILYRLACDEIPSFYRINANCVSRDVMKYFKTRWKDIISGKMSIPAYKSNQPINIHNKYVVPIDVDGDIGITLSLLSSDRKKALDLETGMFRFKVWHKCESSIPILHRCLTGEYKIGESSLQYDKSKRMWELALSYHFEKREHELDPERILGIDLGICIPVVVAVSDSYKRLFVRGNEVTAFREKTEAMRRQMSRARVFAGEGSVGHGRNIRAKPVDRIGHRIANFHNTKNDAWSREIVRFAVKNQCGTIQMEDLSGISSGDAPRFLKNWTYFDLQQKIKYKAEAEGIRVVMVAPKFTSQRCSRCGYISSENRPTQEQFVCQSCGYTANADYNAARNLAIKNIDQIIKKECEPKANRKS

SEQ ID NO: 31 - protein, 493 aa, unidentified
  REGION 1..493: note = uncultivated organism
  REGION 1..493: note = MG119-2 Effector
MGKTDIKEEKIFIGSKKVKITIVEDDPDERNRKYKLIHDEMYKQYLALNMCMSLLATQNTLRMYNSGAENRLLSQIAKLDKKISEKQQIADKDANTEKEQTRKQKAIEDIVKLEEEKNKLREEFDKKATNRTDIDVKFHEQYTSDLYQIIRNEFDFASSITAGLVARKAKDDFKNSFSMGYAKGERSLISYKRSFPLMIRGNEDACYDSNKRKWLDFYYDGDDILIRWLDGIIFKAILGNNPNRNMEIKHTLHKIITGEYRVSQSSIEFDDKGHMYMNVSYRFKKNSKTEIIPGRTLGVDLGIATPAYVCLSDTTYVRSGIGSANDFFRIRQQMKSRRRSLYKSLSLVKGGKGRKKKLQAINSLRDKERNFAKTYNHQLSNKIVKFAKDNGCEFINLEHLTKNGFEDAILSSWSYYELQNMIEYKAEREGIKVRYVDPAYTSQTCSKCGHVDKENRLTQAKFKCVNCGLELNADHNASINIARSDNFVDKKKK

SEQ ID NO: 32 - protein, 486 aa, unidentified
  REGION 1..486: note = uncultivated organism
  REGION 1..486: note = MG119-3 Effector
MATEYICITRKIEVHLHKHGGSEEAVQRLKDEYQIWDTINDNLYKAANRIISHCFFNDAYEYRLKIHSPRFKQIENLLRYSKKNKLSEDDIKELKAERKLLYSDFKKQRLDFLQGYGGKGSEQNSTYQVVTHEFLDVIPSDILTCLNQNIAGVYKQYSLDVEMGRRTIPNFKKGIPVPFAIKKHQQLRLRKRDDGSIYVLLPKGLEWDLSFGRDKSNNREIVERVLSGQYDVGNSSIQESKNGKRFLLLVVKIPKESRVLNKERVVGVDLGIATPLYAALNDNEYVGLSIGSRDQFLKVRMRMAAQKRELQRNLCHTTNGGHGRTQKLQALERLEGKERNWVHLQNHIFSKSIIEYALKNDAGVIQMERLTGFGHDKNDEVDVEYKFILRYWSFFELQSMIEYKAKAADIEVRYINPYHTSQTCSFCGHYEKGQRINQSTFICKNPDCTKGKGKQKSDGSYEGINADWNAARNIALSNKIVDRKKK

SEQ ID NO: 33 - protein, 490 aa, unidentified
  REGION 1..490: note = uncultivated organism
  REGION 1..490: note = MG119-4 Effector
MPIITRKIELKIVKDGLTDEEYDQQWKYLYQINNTIYLAANRISTHCLFNDEYEMRLRLPYKSRYDKINKGLENIKTELEKLNTKKKTSDKEKRDRLLNKQRQLVDERNELDEDVKNKKKDFFQCSKQNSTYQLASKEFLKYIPAEILTDLNRYVQNNHNNNKNKVKSGERALSTYKKGMGIPFSIKPESGLRLFVKEEGIYLKWFKGILFRLEFGKDTSNNRCIVERLIESDKQQKKNKGEDYVANNSSIKLIKKGNDKSTRIFLLLSIDIPAKKQVLDKGVVLGVDLGIKCPLYLAINKNDNFKMQIGDIEHFHNQRTMFQKRFKSLQKLICTQGGHGRKKKLEPLEKLKEKERNWVHTQNHVYSREVIKQALKQNARTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 34 - protein, 462 aa, unidentified
  REGION 1..462: note = uncultivated organism
  REGION 1..462: note = MG119-5 Effector
MTTYTAKIQIVKPLDTDWNTLGKILNELQYETWKFSNKAMQMLWEFDGIGYEYFNKYKKYLNLKEYPFIKDDGKKITTIDSHINNQLKELVKHMNSAGVQATIKNVIDKWKADKKDVQYGRKSVANYKRDLPIELHNKQMFTHDKKPNITIDSRGKYIIEIKLISQKYAMELGRKGTETGFKLLLAVKGNYLKSIVDRVISGEYKLSMSKIVRDKRKKKWFLNLAYTFTPKKKQLNPNRIMGIDMGVNIPAMLAISDSPYYRQAVGSKEEVESFRRQIEARRRSIQRQRKWCGEGSIGHGVKTRLKPLEKLSGKIARFKDTKNHCWSRYIINEAIKNNCGTIQMEDLTGIAKDKAFLKNWTYDDLRQKITYKAEEAGIKVVIIDPYKTSSRCHKCGHIHSSKNKDDWRPKQEKFICESCGFKTNADWNAAKNIATKGIEKIIKKQMEKQDQEYKHGLKYVVN

SEQ ID NO: 35 - protein, 457 aa, unidentified
  REGION 1..457: note = uncultivated organism
  REGION 1..457: note = MG119-6 Effector
MAKASSIITRKIELIPDIEGMTQKESNDRCYGLIRGYDSNLYKVANLLVSQLYGVESLFQMLCLQNNEYMDCKRKLCNKSLSDDERRKVEERFEAVKEAITKRVRELAPQPAQTFAYRAATNSEYSKFFPSDILNSLKQDIYKHYNASKRDQQMGERSLATYKKGMPIPFMLEKHNVIKEIDEDYYLFWFEDTRFRLNFGRDRSNNRAIVYNCINTQKYKLNKMADIQIKNKKLFLIISVEIPKEQREAIKGKVMGVDLGVVHPAYAAVNEGPERSLVGNGEAFQKQRDVFRRRFRELQRCQLTQGGHGRKHKTKATERLRGKERNWVQTENHRISREVVNLAIKWKVETIQMEHLKGFGKNSDGDPSKEHKRILGRWSYFELQKDIEYKAAKVGINVKYVNPAYTSQTCHHCGERGDRSERDFFVCTNENCICYNNRIDADWNAAVNIAKSKDFIK

SEQ ID NO: 36 - protein, 315 aa, unidentified
  REGION 1..315: note = uncultivated organism
  REGION 1..315: note = MG119-7 Effector
MSIPFPWNHTIRIEVVEKENKEETEKRNRRDNEEYDFYLIWYNGIRFRLNFGKDRSNNYQIVKRCFKLDENCNDNYQLKTSSIKMVNGTKEMYLLLVVDIPKEKHDLNNKIVLGVDLGINTPAYVATNVTEDRKAIGSREHFLNSRMAFSRRFHSMQKLQGTANGKGRAKKLEPLERLRGKERNWVHTQNHLYSREVVNFAIKVKAATIQMEDLSGYGKNEEGTVKEEKKFLLGKWSYFELQTMIMDKALKAGITVNFIKPAYTSQACSWCGERGIRDGTTFYCQNPNCKQHEKKNINADHNAARNIARSTEIVK

SEQ ID NO: 37 - protein, 464 aa, unidentified
  REGION 1..464: note = uncultivated organism
  REGION 1..464: note = MG119-8 Effector
MIITRKIELYVHADSKDKKDLDWQMLRYLDNTIYKMANDVISHQFLNQNLSSRLLVHNKNYKPLKKEIDIIEENLNSELSKENKTKLLLNKKKLYSKLTILKKEVDERTKDILNTSIQNTTYQVLSDKYSLVHSQIRTCLNSIVQKNYSNDIYDVLSGKKSLATYKKGMPIPFQKPEFKKNKFDIYFTFPGKINFGLHFGKDKSNNKIIVDNIISGKYKSCDSSIQIKDKKIILLLAVDIPENKIKLDKKITVGVDLGINTPAYAALSNNVCGLAIGKRDEFLNVRLRLQKSRRDMQKNLSFTIGGRGRNKKLQALDRYSEKERNFVKTQNHIISRSIIDFAIENSAGVINLEDISGITKDNTFLNRNWSYFELQTNIEYKAKQNGIEVKYINPAYTSQICSFCGHHSSTQRDKERFTCENDNCTNKDKHGNNKTINADYNSARNIAKSTNFQEKGTNKSKKDL

SEQ ID NO: 38 - protein, 472 aa, unidentified
  REGION 1..472: note = uncultivated organism
  REGION 1..472: note = MG119-9 Effector
MPIITRKIELKIVKDGLTDEEYDQQWKYLYQINNTIYQAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKNKGEDYVANNSSIKLVKYGKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRCGEKINADFNAARNIAMSKKIVKDN

SEQ ID NO: 39 - protein, 471 aa, unidentified
  REGION 1..471: note = uncultivated organism
  REGION 1..471: note = MG119-10 Effector
MPIITRKIELKIVKDGLTDEEYDQQWKYLYQINNTIYLAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKKKGEDYVANNSSIKLVKYGKSTRIFLLLSIDIPAKKQVLDKDVVLGVDLGIKCPLYLAINKNDNFKMQIGDIEHFHNQRTMFQKRFKSLQKLICTQGGHGRKKKLEPLEKLKGKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 40 - protein, 453 aa, unidentified
  REGION 1..453: note = uncultivated organism
  REGION 1..453: note = MG119-11 Effector
MSKITRKIEIIPDIEGLTHDESNKKCYGAFYSFDKNLYKVANLLVSQLYGLDNLLSLMRLQNDEYVKCQYKLSLKSTTDAEKENLKKRMEKIDAELVSIKNGMAPKHPQTFAYRAVTNCVYAKNIPSDILNTLKQDVYKHFNDTKKEQFRGERSLTTYKRGMPVPFSIEKKHAIVCDGDNYYLPWFEDTRFRLNFGRDKSNNRAIIDNCIKTKRYKLCAAAKMQFKDKKLFLLVTVDIPATETTTIKGKVMGVDLGVVNPAYVAVNDGPERSRIGNGEAFQKQRDVFRRRFRELQRSQLTQGGHGRKHKTKATETLRGKERNWVQTENHRISREIVNLASRWKVEYIQMESLKGYGKNQEGEVEDKHKRLLGRWSYFELQKDIEYKAAMVGIQVKYINPAYTSQTCHVCGQRGNRIERDTFICTNPECTCYNQAQDADMNAAINIAKSKDVVK

SEQ ID NO: 41 - protein, 483 aa, unidentified
  REGION 1..483: note = uncultivated organism
  REGION 1..483: note = MG119-12 Effector
MPVITRKIKLNLCTEGLSEDERKAQWKMLYRINDNLYRAANNISSKLYLDEHVSSMVRLKNAEYTSLISDLMKAKKAEDEAVITDLETKIESLKSEMTAQEEAICSYATEMATRTLAGKFASELDLDIYGQILAEVKSVVFKNFNSDSKEVREGKRSIRTYKKGMPIPFPWNKTIRLEAVKKELSGKHDEDEYDFYLNWYKSSRTDKKAIRFRLHFGKDKSNNLLIVKRCLHIDKTSSENYQLQTSSIQMKKGPEGAELYLLLVVNIPQEQHVLNKKIVVGVDLGINVPAYVATNCTEERKAIGDRDHFLNTRTAFSRRFHSFQRLKGTAGGKGRKKKLEPLEQLREKERNWVHTQNHHISRDVINFALQARAATIQMEDLSGYGKDEEGNVKPENKFLQSRWSYFELQSMIKYKAAKCGIKVNLIAPAYTSQTCSWCGQMGLRESTSFVCQNPECKQYGKDIHADYNAARNIARSNKIVKNE

SEQ ID NO: 42 - protein, 453 aa, unidentified
  REGION 1..453: note = uncultivated organism
  REGION 1..453: note = MG119-13 Effector
MSKITRKIEIVPDVEGLTHEESNKKCYNTFYNFDRKLYKVANLLVSQLYGLDNLLPLMRLQNEEYVDCQRKLSRKSITDAVKDEIKKRMEDIDAKLLSVKNEIAPMHAQSYSYRAVTSSECAKDIPSDILDSLKQDVYKHFNENKKEQIRGERSLTTYKKGMPIPFNLKKKHVIISVGDNYYLPWFEDTRFRLNFGRDRSNNRAIIDNCIKAKKYKLCPTAKIQLKDRKLFLLVTADIPETENVPVKGKVMGVDLGVVNPAYVAVNDGPERSRIGNGDAFQKQRDVFRRRFRELQRSQLTQGGHGRKHKTKAIEVLRGKERNWVQTENHRISREIVNLAVRWKVETIQMESLKGFGKNQEGEVENNHKRLLGRWSYFELQKDIEYKAAMAGVAVQYVNPAYTSQTCHVCGQRGNRIERDTFICTNPKCTCYNQAQDADMNAAINIAKSTDVVK

SEQ ID NO: 43 - protein, 486 aa, unidentified; identical to SEQ ID NO: 32
  REGION 1..486: note = uncultivated organism
  REGION 1..486: note = MG119-14 Effector

SEQ ID NO: 44 - protein, 469 aa, unidentified
  REGION 1..469: note = uncultivated organism
  REGION 1..469: note = MG119-15 Effector
MPTITRKIELHLCTEELSDEQQKAQRLLLYHINDNLYKAANNVSSKLYLDEHVSSMVRLKHDEYLSLLRELARAEKKADDELATQLREKLVAAEREMTDQELAICKYATEMSTQSLSYRFVTELETKIFAKILDCLKQGVYATFNSDSRDVKRGERAIRNYKKGMPIPFAWDKSVRIEYEEKEKDFFLRWYNDIRFKFHFGRDRSNNRLIVSRCMKLDKDYEGDYQLCNSSIQIVKRDGSTKYFLLLVVKIPQEHVELNKKIVVGVDLGINYPAFAATNCTEERMSIGDREHFLNTRMQFQRRFKSLQRLKGTTGGKGRNKKLEPLERLRKAEHNWVHTQNHLFSLKVVNFAVQAHAATIHLEDLSGFGKDDDGNADERKEFVLRNWSYYELQNMIKYKAKKFGIQVEKIRPAYTSQTCSWCGQRGFRQGITFICENPECKKCGEKENADYNAARNIANSKDIIKDKDE

SEQ ID NO: 45 - protein, 396 aa, unidentified
  REGION 1..396: note = uncultivated organism
  REGION 1..396: note = MG119-16 Effector
MPKITRKIEIVPDIEGLTHEESNKKCYKTFYNFDRKLYKVANLLVSQLYGLDNLLSLMRLQNDEYVKCQSKLSLKSTTDTTKEDIKKRMKEIDAKLVSIKNDIAPKHPQTYSYRAITSSEYAKDVPSDILNTLKQDVYQHFNENKKEQIRGERSLTTYKKGMPIPFSLEKKHVIVCDGDNYYLPWFEDARFRLNFGRDRSNNRAIIDNCIKTKKYKMCAAAKIQLKDRKLFLLVTVDIPETENVPVKGKVMGVDLGVVNPAYVAVNDGPERSRIGNGDAFQKQRDVFRRRFRELQRLQLTQGGHGRKHKTKATEALRGKERNWVQTENHRISREIVNLAVRWKVETIQMESLKGFGKNQEGEVENNHRRLLGRGLISNFRKTSSIRLPWQVLQYNM

SEQ ID NO: 46 - protein, 489 aa, unidentified
  REGION 1..489: note = uncultivated organism
  REGION 1..489: note = MG119-17 Effector
MPIITRKIELKIVKDGLTDEEYDQQWKYLYQINNTIYLAANRISTHCLFNDEYEMRLRLPYKSRYDKINKGLENIKTELEKLNTKEKTSDKEKRDRLLNKQRQLVDERNELDEDVKNKKKDFFQCSKQNSTYQLASKEFLKYIPAEILTDLNRYVQNNHSNNKNKVKSGERALSTYKKGMGIPFSIKSESGLRLFVKEEGIYLKWFKGILFKLEFGKDSSNNRCIVERLIESYKQQKKKGEDYVANNSSIKLIKKGNDKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 47 - protein, 475 aa, unidentified
  REGION 1..475: note = uncultivated organism
  REGION 1..475: note = MG119-18 Effector
MPTITRKIELKLCTEGLSEEERKAQWKLLYHINDNLYRSANNISSKLYLDSHVGSMVRLKHKDYLETLTALSKAQKKDDTALITELEARLSLFKSEMSDQEIAICKYATEMSERTLADHFASELELDIYSQILTQVKDVVQKYFKDDSKEVSQGLRSIRTYKKGMPIPFPWRNNIWLEAVEAKKQRGNDKKYDYFIKWYGGIRFQLMFGKDRSNNEQIVRRCLKMDENCNDNYQMRTSSIQMKKGAHGTELFLLLVVDIPQEQHKLNNKIVVGVDLGINVPAYVATNVTYDRCAIGSSEGFLAKRNEFRNRFRSLQKLKGTAGGRGRTKKLEPLERLRDKERNWVHTQNHLYSRDVVNFAIKVKAATIQMEDLKGYGKDENGEVVEEKKFLLGRWSYYELQKLIQYKAEKAGMKVNYINPRYTSQTCSCCGVRDPEARNGVHFICRNPACEMYGKEEHADYNAAVNIARSKDINK

SEQ ID NO: 48 - protein, 453 aa, unidentified
  REGION 1..453: note = uncultivated organism
  REGION 1..453: note = MG119-19 Effector
MSKITRKIELIPDIENLTHEESNQRCYKVFYNIDNKLYKVANLLVCQLFGLDNLLSLMRLQNDEYVKCQSKLASKSISEETKRDIKKRMEAIDKELLARKDEIAPKHPQTFAYRAIKDSDYAKDLPSDIFNTLKQDVFKHFNETKKEQLRGERSLTTYKRGIPVPFNLMKKNVIVSDGDNYYLTWFEETRFRLNFGKDRSNNRDIIDNCLKTNKYKLCTAAKIQLKNKKLFLLVTVDIPETKNKIIKGKVMGVDLGVVHPAYVAVNDGPERSLIGDGDAFQKQRDVFRRRFRELQRCQLTQGGHGRKHKTKATENLKGKERNWVQTENHRISREIINLAIRWRVETIQMENLKGFGKDSDGEVGTKHQRLLGRWSYFELQKDIEYKAAMAGIKVVYVNPAYTSQTCHVCGERGDRTERDTFICTNTGCDCYGKPQDADMNAAINIARSKDMIK

SEQ ID NO: 49 - protein, 462 aa, unidentified
  REGION 1..462: note = uncultivated organism
  REGION 1..462: note = MG119-20 Effector
MPIITRKIELKPDIDGLSNEESKNKCYELLRELDNTIYKAANLLVGQMFGMEEFVTMMRLQNDEYVTICKQMKLKKTTEDQRKQLKVRLDEIDAQINVQRQEMAPKGMRTFAGRAVKRSEYANSVPGEILECLKEVVFRHYSDVTKERRKGERTLSTYKKGMPIPFRFWGAQRLVQSEDKFYLQWYNGTRFLLDFGKDRSNNKLIVQRCLGIAEDEISYKTMSSLQLKKNKIFLLLNVEIPKTKFSPIEGKVMGVDLGIVWPAYVAVNNGPEHEHIGDGKNIQNMRNMFWRRFRELQRCQLTQEGHGRKHRTKALEHLHEKERNWIKTENHRISHRIVELAKRWQVETIQMENLKGFGKDKNGNVDDQHKRVLERWSYFELQKDVEYKADIAGIKVRYVNPAYTSLRCSKCGKVGVRPERELFFCTNVECDCYNRSIKADENAARNIAKSNDIIENKDNVEN

SEQ ID NO: 50 - protein, 344 aa, unidentified
  REGION 1..344: note = uncultivated organism
  REGION 1..344: note = MG119-21 Effector
MPTITRKIELKLYTEGLSEDERKAQWGLLYHINDNLYKAANNISSKLYLDDHVASMVRLKHDEYLSLVKALKKAKRQKSPDEDLIAELDKQVSVAEKEMTDQELAICKYATEMSTQSLSYRFATEIEVNIFAKILDCLKQGVYATFNSDAKDVKRGERAIRNYKKGMPIPFAWDDSLKIETEDKEFFLRWYNGLRFRLEFGKDRSNNRLIVKRCLKMDSDFDGEYKLCNSSIQFVKRDGGTKLFLLLVVNIPQENVELNKKIVVGVDLGINSPAYVATNITMERQQIGRRDTFLNARMAFQRRYKSLQKLQNTAGGRGRKKKLEPLERLKDAERNWVRTQKPPF

SEQ ID NO: 51 - protein, 478 aa, unidentified
  REGION 1..478: note = uncultivated organism
  REGION 1..478: note = MG119-22 Effector
MPTITRKIELDLCKDGLSEEQQKEQRLLLYHINDNLYKAANNVSSKLYLDEHVSSIVRLKHAEYLDLLKELAKAEKKNNADLIAELKKKLAAAEKEMTDQELAICKYATEMSTDTLSYRFANEIETKVFGQILATMKQGVHANFQNESKDVKRGKRSIRTYKKGMAIPFPWNDSIRLEYQKKESKKGLGEDGEYEFYLYWFKGIKFQLRFGKDRSNNRLILKRCFKLDENCDDDYHIKTSSIKMVKGSKGTEYYLFLVMDIPQEKHVLNKKIVVGVDLGINFPAYVATNITEERKAIGDRDHFLKARMAIIRRKQSFQRLTGTTGGRGRTKKLEPLERLKEKERNWVHTQNHLFSRDVIKFALQVKAATIQMEDLSGYGRDDEGNVEESKKFLLGKWSYYELQSMIKYKAQKAGIKVNFVNPAYTSQTCSECGKIQPQARNSVHYICENPECKNHGKDIQADYNAARNIARSKNIVKS

SEQ ID NO: 52 - protein, 467 aa, unidentified
  REGION 1..467: note = uncultivated organism
  REGION 1..467: note = MG119-23 Effector
MLTITRKIELHLCTEGLTDEQQKEQRLLLYRINDNLYKAANNVSSKLYLDEHVSSMVRLKHDEYLSLLRELARAEKKADDELTTELREKLAAAEREMTNQELAICKYATEMSTDTLAYRFANEIETQVFGQILACLKQNVHSTFNKDAVDVKRGERAIRNFKKGMPIPFPWSKSIKIEEEKNDFYLRWYNDIRFKFHFGRDRSNNRLIVSRCLKLDKDYEGEYQLCNSSIQIVKRDGSTKYFLLLVVKIPQEHVELNKKIVVGVDLGINYPAYVATNCTEERMSIGDREHFLNTRMQFQRRYKSLQRLKGTAGGKGRSKKLEPLERLRNAERNWVHTQNHLFSLKVVNFAVQTHAATIHLEDLSGFGKDDDGNADERNEFVLRNWSYYELQSMIEYKAKKYGIKVEKIRPAYTSQTCSWCGQRGFRQGVTFICENPECKKCGEKENADYNAARNIANSNDVIKEKNE

SEQ ID NO: 53 - protein, 484 aa, unidentified
  REGION 1..484: note = uncultivated organism
  REGION 1..484: note = MG119-24 Effector
MPTITRKIELRLCSEGLSEEERKAQWSLLYHINDNLYRSANNISSKLYLDEHVSSLVRLKHKEYKKLIKDLAKAKKQKIADAKAIAEMEDKLKSYEHEMTEQELAICKYADEMSSLSLAYGFATELELKIYAQILTQIQSKVHSDFQNDQKEVREGKRSIRTYKKGLPIPFPWNDTIKIEVVKKNNTEEQAKKSRRDNEEYDFYLNWYNGMRFRLHFGKDRSNNYQIVKRCFKLDEYCLDNYQLKTSSIQLVRKNKKTDFYLLLVVDIPQEKYSLNNQIVVGVDLGINVPAYVATNVTEDRKSIGNREHFLNARMAIKRKFHSLQRLEGTAEGRGRKKKLEPLERLREKERNWVHTQNHLFSRDVIKFALQAKAATIQMEDLSGYGKDDEGNVEEGKKFLLGNWSYYELQSMIKYKAQMVGMKVNFVAPAYTSQTCCCCGVKDEKNRNSTSFVCHNPQCTMYMKDIHADYNAARNIAKSKEIVK

SEQ ID NO: 54 - protein, 326 aa, unidentified
  REGION 1..326: note = uncultivated organism
  REGION 1..326: note = MG119-25 Effector
MERGIRTIPNFKKGIPVPFSIKPNGEIALKKRDDGTIYVRFPKGLEWDLNFGRDRSNNREIVERVLSGQYGVGNSSIQESKNKKQFLLLVVKIPKENRVLDKERIVGVDLGVNTPLYAALNDNEYGGMGIGSREQFLKVRERMNAQKRELQRSLRHSTNGGHGRSQKLQALDRLEGKERNWVHLQNHIFSKSIIEYALKNDAGVIQMERLTGFGRDNNEEVQNEYKYILRYWSYFELQTMIEYKAKAAGIEVRYINPYHTSQTCSFCGHYEKGQRINQSTFICKNPDCTKGKGKQKSDGAYEGINADWNAARNIARSNEFVGKKKK

SEQ ID NO: 55 - protein, 469 aa, unidentified
  REGION 1..469: note = uncultivated organism
  REGION 1..469: note = MG119-26 Effector
MPIITRKIELKIVRLTDEEYDQQWKYLYQINNTIYQAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKNKGEDYVANNSSIKLVKYGKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKHNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 56 - protein, 481 aa, unidentified
  REGION 1..481: note = uncultivated organism
  REGION 1..481: note = MG119-27 Effector
MATEYTCITRKIEVHLHKHGDDEESVSRYKIEYQKWDEINDNLFKAANRVVSHHFFNDAYEYRLKLQSKRFRDIEKQLRTSRKNNLVDEEITALRAERDKLSAEFKKQRLEFLGVSEQHSTYRVITNEFIDVIPSDVLTCLNQNISLTYKNYAFDVERGIRTIPNFKKGIPVPFSIKGHGQLLLKKREDGSVYIRFPLGLEWDLRFGRDRSNNREIVERILSGEYDAGNSSIQETRNGKRFLLLLVKIPKEKHVLNPNRVVGVDLGINIPLYAALNDNEYGGMSIGSREQFLKMRMRMFAQKRELQSNLRHSTNGGHGRGQKLQALERVEGKERKWVRLQNHIFSKSIIDYALKNGAGVIQMERLTGFGRDKNDEVDEESKFILRYWSFFELQTMIEYKAKAAGIEIRYVNPYHTSQTCSFCGHYEKGQRINQATFICKNPDCIKGRGERKADGTFEGINADWNAARNISLSREFMNRKKK

SEQ ID NO: 57 - protein, 488 aa, unidentified
  REGION 1..488: note = uncultivated organism
  REGION 1..488: note = MG119-28 Effector
MATEYTCITRKIEVHLHRHGEYEEAKQRLIDDYRVWDTINDNLYKAANRIVSHCFFNDAYEYRLKIHSPRFQEIEKLLKYPKRNKLTDEDIKQLKAERKQLFADFKKQRHTFLRGGVAEGANPEQNSTYKVISNEFLEVIPSEILTNLNQNISSTYKNYSLDVERGIRTIPNYKRGIPVPFSIKQRGELMLKSRDDGSIYVRFPLGLEWDLSFGRDRSNNREIVERVLSGQYDVGNSSIQESKNRKRFLLLVVKIPKENHNLNPDRIVGVDLGINIPLYAALNDNDYGGMGIGSREQFLNMRMRMDAKKRELQRNLRQSTNGGHGRKQKLQALERFEGKERNWVHLQNHIFSKSIIEYAVKNNAGAIQMERLTGFGRDKNDEVDSNFKFILRYWSFFELQTMIEYKANAAGIEVRYVDPYHTSQTCSFCGHYEKGQRLNQSTFVCKNPDCEKGKGKKLSDGTYQGINADWNAARNIALSDKIVDRKKK

SEQ ID NO: 58 - protein, 493 aa, unidentified
  REGION 1..493: note = uncultivated organism
  REGION 1..493: note = MG119-29 Effector
MIHLHFQNRTIIMTIITRKIELKHCKENLSEEERISQKKLLLHINDNLYRAANNISSKLYLDEHVSSLVRLKHKEYKDLVRDLAKAKKQKSHDEASIAEMEARLKGYEDEMTSQERAICKYTDEMSPLSLAYGFATELELDIYAQILTQIQSKVHKDFTNDNEDVRDGKRSIRTYKKGMPIPFPWNNAIRIEAIEKAKTEEKEVKNRRNREDYDFYLNWYNGIRFRLHFGKDKSNNYQIVKRCLKLDENCRDNYQLKTSSIKMENGTKKMYLLLVVDIPQEKYKLNNNIVLGVDLGINVPAYVATNITEDCKAIGNREHFLNARMAISRRFHSLQRLQGTAEGRGRKKKLEPLEHMREKERNWVRTQNHLYSRDIVNFAIKVKAATIHMEDLSGYGKDDEGNVEEDKKFLLGKWSYFELQTMIKNKAQKAGIRVNMVKPAYTSQTCSWCGERGIRDGLSFFCQNPECKQYAKKDIHADYNAARNIARSKDIVK

SEQ ID NO: 59 - protein, 308 aa, unidentified
  REGION 1..308: note = uncultivated organism
  REGION 1..308: note = MG119-30 Effector
MEIPFSIRENKRLKLFIKEKGIYLKWFKEILFRLEFGKDASNNRCIVERLIESDKQQKNKGEDYVANNSSIKLVKNEKNTRIFLLLSIDIPAKKQVLDKEVVLGVDLGIKCPLYLAINKNDNFKMQIGDIEHFHNQRTMFHKRFKSLQKLMCTQGGHGRKKKLEPLEKLKEKERNWVHTQNHVYSREVIKQALKQNAGIIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 60 - protein, 453 aa, unidentified
  REGION 1..453: note = uncultivated organism
  REGION 1..453: note = MG119-31 Effector
MSIVTRKIELIPDIENLTHEESNQRCYKLLYNIDKKLYKLANLLVCQLFGLDNLLSLMRLQNDEYVKFQSKLASKSISKETQKNIKERMKEIDKELLARKAEIAPKSPLTFAYRAIKGSLYAKDLPSDIFNTLKQDVFKHFNETKKEQLRGERSLATYKRGIPIPFSLMKKNVIVSEGDNYYLTWFEETRFKLNFGKDRSNNRAVIDNCLKTNKYKLCTAAKIQLKNKKLFFLVTVDIPETKNTIIKGKVMGVDLGVVHPAYVAVNDGPERSLIGDGDAFQKQRDVFRRRFKELQRCQLTQGGHGRKHKTKATEILRGKERNWVQTENHRISRKIVNLAIRWKVESIQMENLKGFGKDSEGEVETKHKRLLGRWSYFELQKDIEYKAQMAGIKVVYVNPAYTSQTCHVCGKKGDRTERDTFICLNTECSCYGKPQDADMNAAINIARSKNIVK

SEQ ID NO: 61 - protein, 488 aa, unidentified
  REGION 1..488: note = uncultivated organism
  REGION 1..488: note = MG119-32 Effector
MATEYTCITRKIEVHLHRHGEGEEAIERMKREYQIWDDINNNLYKAANRIVSHCFFNDAYEYRLKIHSPRFKAIEKALAYAKRDKLTPDEVKQLKAERSALFKQFKSQRQTFLRGGEEEGSNPEQNSTYQVITKEFKDYIPSEVLTNLNQNIAGVYKQFSLEIQNGNRSIQNFKKGIPVPFAIKSHGVLMIKKREDGTIYVRFPLGLEWDLAFGRDRSNNREIVERILSGAYDVGNSSIQEAKNGKRFLLLVVKIPKEINKLDKNRVVGIDLGINIPISAALNDNAYGGMSVGSREQFLKVRMRMAAQKRELQRSLRSTTNGGHGRKQKLQALDRLEGKERNWVHLQNHIFSKTIIDYAIKNGAGVIQMENLSNFGHNKFDEVNEGYKFILRYWSFFELQTMIEYKANAAGIDVRYVNPYHTSQTCSFCGHYEKGQRINQSTFVCKNPECTKGKGKQRGDGTFEGINADWNAARNIALSNDIVAKKKK

SEQ ID NO: 62 - protein, 469 aa, unidentified
  REGION 1..469: note = uncultivated organism
  REGION 1..469: note = MG119-33 Effector
MPTITRKIELKLCTEGLSDQERKDQWNLLYHINDNLYRAANNISSKLYLDDHVGSMVRLKHAEYLSLLRALEKAKKQKAPDEELIAELSQQVATAEQEMDEQAKAICQYATEMSTQTLSYRFATEMETNIFGQILTCLRQGVFSTFNSDARDVKRGERSIRTYKKGMPIPFPWNDSLRIELENGEFFLRWYNGMRFRFDFGKDRSNNRLIVQRCMKMDEDYEGEYKLCNSSIQMVKREGKPKFFLLMVVNIPQEQVELNKKIVVGVDLGINAPAYVATNITPERKQIGDREHFLNERMAFQRRFKSLQRLKGTTGGRGRAKKLEPLERLRKAEQNWVHTQNHLFSREVIDFAVKARAATIHIEDLSGFGKDQDGNADEKKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 63 - protein, 471 aa, unidentified
  REGION 1..471: note = uncultivated organism
  REGION 1..471: note = MG119-34 Effector
MPIITRKIELKIVKDGLTDEEYDQQWKYLYQINNTIYLAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKNKGEDYVANNSSIKLVKYGKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 64 - protein, 483 aa, unidentified
  REGION 1..483: note = uncultivated organism
  REGION 1..483: note = MG119-35 Effector
MPLITRKIEVQIYSKGVSAEDYNSQWAFLRQINNYLYLAANLLASHNYFEEILSDRLKVTDTEYYKKRIKMSRQSGSGKEESKKELREIDKEVRNKINELFKGTTKRTELYRIVAEKFGEVIPTKILANLNKSVTDCYVGEEKDIRKGERSLRNYKKGLPIPFSWGKKEPTFPYIVKNEETGNSDILFQFFPDLSKKFCKRQNPGSAGSPLTFRMHFGKDKSNNRAIVERIIADKDKAGDDRYKLSQSSLQIVGKDKNTKFFLLLCVDIPAQKNHLDPDKVLGIDLGINYPVYYATNKEDNIKGHIGDRDSFLKQRLAFQRRYKALQSVTCTSGGRGRKKKLQPLESLRSKERAWTKHMNHLYSREIIKAAQKLGTGTIHMEELTGIGKDEDGNVKKKRQFILRNWSYFELQTMIQYKAKMAGITVKKIPPAYTSQTCSCCGEKGERKEQAHFKCLNPLCSMFDKDVNADYNAARNIAKYQAD

SEQ ID NO: 65 - protein, 468 aa, unidentified
  REGION 1..468: note = uncultivated organism
  REGION 1..468: note = MG119-36 Effector
MPTITRKIELTLCTDGLSDEERKAQWGLLYHINDNLYKAANNISSKLYLDEHVSSMVRLKHDEYLSLQKELAKAERQKMPDVDVIEELRERLSAVEQEMSDQELAICKYATEMSTNTLAYRFATEIETNIFGQIMARLENNAQAVFLTDAPDVKRGERAIRNYKKGMPIPFPWNNSIKIECEGGEFYLRWYNGLRFHFNFGKDRSGNRLIVQRCLKLDKEYDGEYKLCNSSIQMVKRDGSTKFFLLMVVNIPQEYVELNKHIVVGVDLGINVPAYVATNITPERKAIGDREHFLNTRMAFQRRYKSLQRLKTTAGGKGRTKKLEPLERLRQAEHNWVHTQNHLFSREVVNFALQTHAATIHLEDLSGFGKDSDGNADERKEFVLRNWSYYELQNMITYKAAKYGIRVEKIRPAFTSRTCSCCGHEGFREGVTFICENPECQQFGEKVHADYNAARNIANSKDVIKKNE

SEQ ID NO: 66 - protein, 469 aa, unidentified
  REGION 1..469: note = uncultivated organism
  REGION 1..469: note = MG119-37 Effector
MPTITRKIELVLCTEGLSDEERKAQRGLLYHINDNLYKAAKNISSKLYLDDHVGSMVRLKHDAYLSLLRNWEKAKKQKTPDEELIEELGKQVAEAEKEMTDQELAICKYATEMSTQSLSYRFATELETNIFAKILDCLKQGVFATFNSDARDVKRGERAIRNYKKGMPIPFAWSDSLRIEAEKNDFYLRWYNGLRFYLNFGKDRSNNRMIVRRCLNLDEDFEGEYKLCNSSIQMVKRDGAPKIFLLLVVNIPQEHVELNKKIVVGVDLGVNVPAYVATNITDERKAIGDREHFLNTRMAFQRRYKSLQRLRGTAGGKGRAKKLEPLERLRKAEHNWVHTQNHLFSREVVNFAVQARAATIHMEDLSGFGKDSDGNADEKKEFVLRNWSYYELQTMIAYKAAKYGIKVEKIRPAYTSKTCSWCGQQGFRQGVTFICENPDCKQCGEKVHADYNAARNIANSKDVIKDKNE

SEQ ID NO: 67 - protein, 497 aa, unidentified
  REGION 1..497: note = uncultivated organism
  REGION 1..497: note = MG119-38 Effector
MPTITRKIELKIVKDRLTDEKERLTDEEYDQQWKYLYQINNTIYQAANRISTHCLFNDEYEMRLRLSYKSRYDKINKGLENIKTELEKLNTKKKTSDKEKRDRLINEQRQLVDERNELDEDVKNKKKDFFQCSKQNSTYQLASKEFLKYIPAEILTDLNRYVQNNHNNNKNKVKSGERALSTYKKGMGIPFSIKPESGLRLFVKEEGIYLKWFKGILFKLEFGKDTSNNRCIVERLIESDKQQKKNKGEDYVANNSSIKLIKKGNDKSTRIFLLLSIDIPAKKQVLDKDVVLGVDLGIKCPLYLAINKDDNFKMQIGDIEHFHNQRTMFQKRFKSLQKLMCTQGGHGRKKKLEPLEKLKEKERDWVHTQNHVYSREVIKQALKHNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 68 - protein, 135 aa, unidentified
  REGION 1..135: note = uncultivated organism
  REGION 1..135: note = MG119-39 Effector
MQSAIGYERKNHLFSRDVVRFAVKARAASIHMEDLSGFGKDNDGNADDKKEFVLRNWSYYELQTMIKYKAAKYGIKVEKIRPAFTSQTCSWCGQPGFRRGEIFICENPACEKCGEKENADYNAARNIANSKDIIK

SEQ ID NO: 69 - protein, 479 aa, unidentified
  REGION 1..479: note = uncultivated organism
  REGION 1..479: note = MG119-40 Effector
MPIITRKIELKIVKDGLTDEEYDQQWKYLYQINNTIYLAANRISTHCLFNDEYEMRLRLRYMSQCNKIDKKLEKLNPDKKTSDKEEQYRLLNERKELDEDFKNKKQDFQRIKRNSTYQLVSKEFLKYIPAEILTDLNQYVQNNYNNNKREVKSGERALSTYKKGMGIPFSIKPESGLRLFIKENGIYLKWFKLNNDRKEKEILFRLEFGKDASNNRCIVERLIESDRQQKKKGEDYIANNSSIKLVKNGKNTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRCGEKINADFNAARNIAMSKKIVER

SEQ ID NO: 70 - protein, 468 aa, unidentified
  REGION 1..468: note = uncultivated organism
  REGION 1..468: note = MG119-41 Effector
MPSITRKIELTLCTEGLSDEKRKEQWGLLYHINDNLYKAANNISSKLYLDDHVSSMVRLKHADYLSLLRELARAEKQKTPNIDLIAELREKVSTAEKEMTDQELAICNYATEMSTKSLSYRFATEIETDIFAKILDCLKQGVYAKFTSDTRDVKNGERAIRNYKKGMPIPFAWNDSLRIEEENGEFYLRWYNGLRFLLYFGKDRSNNRMIVKRCLKMDEEFDGEYKLCNSSIQIVKREGRTKLFLLLVVNIPQENVELNKNIVVGVDLGLNYPAYVATNCTEEHKYIGDRDHFLKIRMQFQSRFRSLQRLRGTAGGKGRAKKLEPLERLRQAECNWVHTQNHLFSRDVVNFAVQTRAATIHMEDLSGFGKDPDGNAEEKKEFVLRNWSYYELQNMIKYKAKIYGIRVEKVRPAYTSKNCSWCGHLGFRESTTFICENPECKQCGKKIHADYNAARNIANSKDIIKENE

SEQ ID NO: 71 - protein, 501 aa, unidentified
  REGION 1..501: note = uncultivated organism
  REGION 1..501: note = MG119-42 Effector
MPIITRKIELKIVKDGLTDEKYKLTDEEYDQQWKYLYQINNTIYQAANRISTHCLFNDEYEMRLRLHMPRYKEIDEGLENIKTELEKLNTKKKASDKEKRDRLLNEQRQLVDERKELDKDVKNKKKDFLQCSKQNSTYQLVSKEFLKYIPAEILTDLNQYVQNNYNNNKRKVKSGERALSTYKKGMGIPFSIKPESGLRLFIKENGIYLKWFKLNNDRKEKEILFRLEFGKDASNNRCIVERLIESDRQQKKKGEDYIANNSSIKLVKNGKNTRIFLLLSIDIPAKKQVLDKDVVLGVDLGIKCPLYLAINKDDNFKMQIGDIEHFHNQRTMFQKRFKSLQKLMCTQGGHGRKKKLEPLEKLKEKERNWVHTQNHVYSREVIKQALKQNAGTIHIESLKDFGKGKDGYVKDEYKYLLRYWSYYELQSMIEYKAKLEGIEVKYIDPAYTSQTCSYCGERGERKKQEEFVCTNPQCKRCGEKINADFNAARNIAMSKKIVKDN

SEQ ID NO: 72 - protein, 472 aa, unidentified
  REGION 1..472: note = uncultivated organism
  REGION 1..472: note = MG119-43 Effector
MPIITRKIELKIVKDGLTDEEYDQQWKYLYQINNTIYLAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKNKGEDYVANNSSIKLVKYGKSTRIFLLLSIDIPAKKQVLDKKVVLGVDLGIKCPLYLAINKNDNFKMQIGDIEHFHNQRTMFQKRFKSLQKLICTQGGHGRKKKLEPLEKLKEKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 73 - protein, 470 aa, unidentified
  REGION 1..470: note = uncultivated organism
  REGION 1..470: note = MG119-44 Effector
MPIITRKIELKIVRLTDEEYDQQWKYLYQINNTIYQAANRISTHCLFNDEYEMRLRLRYMSQCNKIDKKLEKLNPDKKTSDKEEQYRLLNERKELDEDFKNKKQDFQRIKRNSTYQLVSKEFLNYIPAEILTDLNQYVQNNHNNNKNKVKSGERALSTYKKGMGIPFSIKPESGLRLFVKEEGIYLKWFKGILFRLEFGKDASNNRCIVERLIESDKQQKKKGEDYVANNSSIKLVKNGKNTRIFLLLSIDIPAKKQVLDKEVVLGVDLGIKCPLYLAINKDDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 74 - protein, 486 aa, unidentified
  REGION 1..486: note = uncultivated organism
  REGION 1..486: note = MG119-45 Effector
MPIITRKIELKIVRLTDEEYDQQWKYLYQINNTIYQAANRISTHCLFNDEYEMRLRLSYKSRYDKINKGLENIKTELEKLNTKKKTSDKEKRDRLINEQRQLVDERNELDEDVKNKKKDFFQCSKQNSTYQLASKEFLKYIPAEILTDLNRYVQNNHNNNKNKVKSGERALSTYKKGMGIPFSIKSESGLRLFVKEEGIYLKWFKGILFKLEFGKDSSNNRCIVERLIESYKQQRKKGEDYVANNSSIKLIKKGNDKSTRIFLLLSIDIPAKKQVLDKDVVLGVDLGIKCPLYLVINKDDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRCGEKINADFNAARNIAMSKKIVER

SEQ ID NO: 75 - protein, 453 aa, unidentified
  REGION 1..453: note = uncultivated organism
  REGION 1..453: note = MG119-46 Effector
MSKITRKIEIIPDIDGITHEESNKKCYNTFYKFDRKLYKVANLLVSQLYGLDNLLSLMRLQNDEYVKCQSKLSFKSITDATKEEIKKRMQEIDAELVSMKNDIAPKHPQTYSYRAVISSEYAKDIPSDILNNLKQDVYQHFNENKKELIRGERSLATYKKGMPIPFSLEKGHVIICDGDNYYLPWFEDIRFRLNFGRDRSNNRAIIDNCIKTKKYKLCASAKIQLKERKLFLLITVDIPKANSVPVKGKVMGVDLGVVNPAYVAVNDGPERSRIGNGETFQKQRDVFRRRFRELQRSQLTQGGHGRKHKTKATEILRGKERNWVQTENHRISREIVNLASRWKVETIQMESLKGFGKNQEGEVENNHKRLLGRWSYFELQKDIEYKAAMAGIKVQYVNPAYTSQTCHVCGQRGNRIEREIQGSNGWYQGTICKSCIYQPNLSCLWSTRKQNRT

SEQ ID NO: 76 - protein, 469 aa, unidentified
  REGION 1..469: note = uncultivated organism
  REGION 1..469: note = MG119-47 Effector
MPTITRKIELHLCTDGLSEEQRKEQWGVLYHINDNLYKAANNISSKLYLDEHVSSMVRMKHAEYLSLVKELARAEKQKTVDEETIEELRDKLATAEKEMTDQEKAICKYATEMPTNTLAYKFANEIETDIFGQILACLDNNAHAVFVDDAPTVRKGERAIRNYKRGMPIPFPWNKAIRIDVSEGELYLKWYHGIRFHLFFGKDKSNNRLIVNRCLKLDKDYEGDYKLCNSSIQIVKREGAVKLFLLLVVNIPQEHVELNKKIVVGVDLGVNVPAYVATNITEERKAIGDREHFLNTRMQFQRRYKSLQRLKATTGGKGRAKKLEPLERLRKAEHNWVHTQNHLFSREVVNFAVQTHAATIHLEDLSGFGKDNDGNADEKKEFVLRNWSYYELQNMIAYKAAKYGIKVEKVKPAYTSKTCSCCGQIGFRQGVTFICENPACKQCGEKVHADYNAARNIANSKDVKKDKNE

SEQ ID NO: 77 - protein, 495 aa, unidentified
  REGION 1..495: note = uncultivated organism
  REGION 1..495: note = MG119-48 Effector
MPTITRKIELKIVKDRLTDEKERLTDEKYDQQWKYLYQINNTIYQAANRISTHCLFNDEYEMRLKLHMPRYKDIEKKLEEIEKKQKGLNTKKKASDKEERDRLLNEKQQLVNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDKQQKGKGEDYVANNSSIKLIKKGNDKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 78 - protein, 410 aa, unidentified
  REGION 1..410: note = uncultivated organism
  REGION 1..410: note = MG119-49 Effector
MVRMKHAEYLSLLSELERARKKTDEELVEVLSNRVAAAEKEMTDQELAICQYATEMSTDTLAYRFANEIEIKVFGQILACLKQGIHSNFKKDAVDVKRGERAIRNYKKGMPIPFPWSKSIRIESEGSEFYLRWYNGIRFRFTFGKDRSNNRLIVRRCLKMDDDFEGEYKLCNSSIQLAKRDGAPKLFLLLVINIPQEHVELNKKIVVGVDLGINAPAYVATNITEERKQIGDRDHFLKTRLTFQRRYKSLQRLKGTSGGKGRTKKLEPLERLRKAERNWVHTQNHLFSRDVVDFAVQTRAATIHMEDLSGFGKDDDGNADEKKEFVLRNWSYYELQNMITYKAAKNGIRVEKIRPAYTSQTCSCCGEKDPRNRDGISFICHNPNCEQYDKEVHADYNAARNIAKSNDIVE

SEQ ID NO: 79 - protein, 471 aa, unidentified
  REGION 1..471: note = uncultivated organism
  REGION 1..471: note = MG119-50 Effector
MPTITRKIKLELCTKGLSEEERKAQWNLLYHINDNLYRSANNISSKLYLDEHVSSLVWLKHKEHQTLKADLAKAKKQKIQDEKTIAELESRLKSCESEMSDQELAICKYTDEMSSKTLSYKFATELELNIYAQILNQVQSKVYADFQNDQKDVRDGKRAIRTYKKGMPIPFPWRNNIRMEPVKKGREYEFYIKWYNDIRFQLIFGKDRSNNRLILQRCFKLDENCVEDYQMRTSSIKMVKGANGTELFLYLVVDIPQEKHTLNNKIVVGVDLGINVPAYVATNVTDDRKAIGDREHFLNTRMAISKRFHSFQRLKGTTGGRGKTKKLEPLERLKEKERNWVHTQNHLFSRDVITFALHVKAATIQMEDLSGYGKDDEGNVVEEKKFLLGKWSYYELQDMIKYKAKKVGMRVNFIKPAYSSQTCSWCGERGERNSTSFVCTNSECSHYGEDLHADYNAARNIARSKNIIRYE

SEQ ID NO: 80 - protein, 488 aa, unidentified
  REGION 1..488: note = uncultivated organism
  REGION 1..488: note = MG119-51 Effector
MATEYTCITRKIEVHLHKHGDGEEAEQRRAEEFRIWNEINDNLYKAANRIVSHCFFNDAYEYRLKIQSPRYNEIQKSLRYTKRNKLTDDEIKSLKAERKSLFDEFKKQRQTFLRGGVSEGANPEQNSTYKVVSNEFLDVIPSHILTCLNQNVSSTYKCYSKEVEFGNRTIPNFKKGIPVPFPIKHGNILLLKKREDGSIFVDFPKGLEWDLSFGRDRSNNREIVERILSGQYDAGTSSLQEGKNGKIFLLLVVKIPKQSNALDPNRVVGIDLGINIPLYAALNDNEYGGMSIGSREQFLKMRMRMTAQKRELQRNLHYSTNGGRGRSHKLQALERLEGKERNWVHLQNHIFSKNIIEFAVKNNAGVIQMERLTGFGHDRNDEVDDGFKFILRYWSFFELQSMIEYKAEAAGIEVRYIDPYHTSQTCSFCGHYEKGQRIDQATFICKNPECEKGKGKKRSDGTYIGINADWNAARNIALSDKFVDKKKK

SEQ ID NO: 81 - protein, 496 aa, unidentified
  REGION 1..496: note = uncultivated organism
  REGION 1..496: note = MG119-52 Effector
MPTITRKIELKIVKDRLTDEKERLTDEKYDQQWKYLYQINNTIYQAANRISTHCLFNDEYEMRLKLHMPRYKDIEKKLEEIEKKQKGLNTKKKASDKEERDRLLNEKQQLVNERKEIDEDVKNKKKDFLQCSKQNSTYQLVSKEFLKYIPAEILTDLNRYVQNNHNNNKNKVKSGERALSTYKKGMGIPFSIKPESGLRLFVKEEGIYLKWFKGILFKLEFGKDTSNNRCIVERLIESDKQQKKNKGEDYVANNSSIKLIKKGNDKSTRIFLLLSIDIPVKKQVLDKDVVLGVDLGIKCPLYLAINKDDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCERRGEKINADFNAARNIAMSKKIVKDN

SEQ ID NO: 82 - protein, 485 aa, unidentified
  REGION 1..485: note = uncultivated organism
  REGION 1..485: note = MG119-53 Effector
MPIITRKIELKIVKDGLTDEEYDQQWKYLYQINNTIYQAANRISTHCLFNDEYEMRLKLHMPRYKDIEKKLEEIEKKQKGLNTKKKASDKEERDRLLNEKQQLVNERKEIDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDKQQKGKGEDYVANNSSIKLVKNGKNTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 83 - protein, 497 aa, unidentified
  REGION 1..497: note = uncultivated organism
  REGION 1..497: note = MG119-54 Effector
MAEYTVITRKIEVHLHSDDNSEESKELLQEKYRAWNEINDHLYEAANRIVSHRFFNDEYEMRLRVHSPRYKEIKDILRKAQKDKLADSYVKELKAERKQLESEFREQRLTFLRGGDPDGTGSEQNSTYDVATNEFSDSIPSNILTCLNQNIIKTYNSYKTEIKFGERTIPNFKKGLPVPFPIKYNGNLQICKRDDGTIFVRLPGKKGQKWHLEWDLDFGRDRSNNREIVERILNGTYDVGNSSIQETRSRKRFLLLVVKIPKERNVSLDPNRVAGIDLGINIPLYAALNDNEYARSRIGSRDQFLDVRNRMEAQLHKLQANLRNSTAGGRGRGHKLQALERFKEKERNWVRLQNHIFSKGAIEFAVKNNAGVIQMERLTGFGRDGNGEVKNDRKFLLRNWSYFELQQLIEYKALAAGIEVRYVDPYHTSQICSFCGHYEPDQRSDQPHFICKNPECEKGKGKKLKDGSYEGINADWNAARNIAKSEKIVDRRKNTKK

SEQ ID NO: 84 - protein, 485 aa, unidentified
  REGION 1..485: note = uncultivated organism
  REGION 1..485: note = MG119-55 Effector
MPIITRKIELKIVKDGLTDEEYDQQWKYLYQINNTIYQAANRISTHCLFNDEYEMRLKLHMPRYKDIEKKLEEIEKKQKGLNTKKKASDKEERDRLLNEKQQLVNERKEIDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDKQQKGKGEDYVANNSSIKLVKNGKNTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNARTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 85 - protein, 467 aa, unidentified
  REGION 1..467: note = uncultivated organism
  REGION 1..467: note = MG119-56 Effector
MPTITRKIELTLCTEGLADEQRKAQWQLLYHINDNLYKAANNVSSKLYLDEHVSSMVRMKHSEYLSLVKELAKAEKQKKTDEDLIAGLRDRLTAVEREMTDQELAICKYATEMSTKTLAYKFATEIETNVFGQILACLKQAAQSNYNSDARDVKRGERAIRNYKKGMPIPFPWNDNLRIEPDGDEFFLRWYNGLRFHLIFGKDRSNNRMIVKRCLKMDKDFEGEYKLCNSSIQMAKSDGKPKLFLLLVVNIPQEHVELNKNIVVGVDLGVNVPAYVATNITEERKTIGDREHFLNTRMQFQRRYKSLQRLKATASGKGRTKKLEPLERLRKAEHNWVHTQNHLFSREVVNFAVQTRAATIHMEDLSGFGKDNDGNADEQKEFVLRNWSYYELQNMIAYKAAKYGIKVEKVRPAYTSKTCSWCGQQGFRQGVTFICENPACKQCGEKVHADYNAARNIANSKDVLNNS

SEQ ID NO: 86 - protein, 474 aa, unidentified
  REGION 1..474: note = uncultivated organism
  REGION 1..474: note = MG119-57 Effector
MAAITRKIELYIDKRNLTEDEYKAQWQYIRQIDNTLFLAANRISSHCLLNDELEIRLKLQMPEYCEIEKSLRNSKKNNLSKEEVSELKLRKKELDAIVKRQKDEFLRTSQNSTYQLVAREFANIPTDILTNLNNDIVSKYGKSRLDIINGVKSLSTYRKGLPIPFSVNKKFPFVFIDGEYYLEWFKGKTKEITGKPLQFKLHFGKDKSNNRTIVERIVESARLGKKKGEAYVVNNSSIQLVEKGNSSKIFLLLSLDIPTAKRTLDPNLVMGIDLGINYPIYYATNGNAYIKGHIGDRDSFLNERMVFQRRFKELQRLQCTQGGRGRQKKLAPLEKLRDKERNWVKTKNHIFSREIIKCALKIGAGIIHLEKLKNIGKDKDGNIEDSKKYILRNWSYHELQERIEYKAAMEGIMVKYVDPAYTSQTCSCCGNKGERISQSVFKCLCPECSEYGKEVNADYNAARNIAKSEIFVKK

SEQ ID NO: 87 - protein, 478 aa, unidentified
  REGION 1..478: note = uncultivated organism
  REGION 1..478: note = MG119-58 Effector
MPTITRKIELRLCTEGLSDEERKAQWMLLYHINDNLYRSANNISSKLYLDEHVSSMVRLKHAEYQSTAAELLKAKKNNADEGTISTLEDKVETLKTEMSAQGIAICNYATEMATRTLAGKFASELELNIYGQILAEVKNVVHTNFTNDAKEVREGKRSIRTYKKGMPIPFPWNKSIKIEPVKASSQNEGQDDYEFYLKWYNGLKFILHFGKDRSNNRQILKRCFGLDNLCNERYQMRTSSIQMKKGSNGMELYLLLVLSIPKEQHSLNKKVVVGVDLGINVPAYVATNCTEERRAIGDREQFLNTRMAIIRRKHSFQRLKGTAGGRGRKKKLDPLERLRETERNWVHTQNHLYSRDIIKFALETKAATIQMEKLKGFGRDDNGNVIEEKKFLLGKWSYYELQNMIKYKAGKVGIKVNFIAPAYTSQTCSCCGVRDDRNRKSTSFICHNPDCQMYGKEIHADYNAARNIARSKNVIKDE

SEQ ID NO: 88 - protein, 466 aa, unidentified
  REGION 1..466: note = uncultivated organism
  REGION 1..466: note = MG119-59 Effector
MPTITRKIELTLCTDGLSDDLRKDQWQLLYHINDNLYKAANNISSKLYLDEHVASMVRLKHAEYLGLIKELAKARKRADDEAVRDLCSKLAVAEQEMNEQAKAICDYATEMSTQTLSYNFAKEIETNIFGQILTCLRQGVLLNFNSDARDVKRGERAIRNYKKGMPIPFPWNDTIKIVSEGDEFYLRWFSGLRFHLNFGKDRSNNRMIVRRCLKMEQDFDEEYKISNSSIQVAKRDGKQKLFLLLVVQIPQEQVVLNKKIVVGVDLGVNVPAYVATNITEERKAIGDREHFLNTRMQFQRRYKSLQRLKTTEGGRGRAKKLEPLERLRKAEHNWVHTQNHLFSREVVNFALQTQAATINMEDLSGFGKDNDGNADECKEFVLRNWSYYELQNMIVYKASKYGIRVQKIRPAYTSKTCSWCGHMGFREGVTFICENPDCKQFGEKVHADYNAARNIANSKEIIKNDE

SEQ ID NO: 89 - protein, 483 aa, unidentified
  REGION 1..483: note = uncultivated organism
  REGION 1..483: note = MG119-61 Effector
MPTITRKIRLHLCTDGLSEEERKAQWKMLYRINDNLYRAANNISSKLYLDEHISSMVRLKHAEYTSLKTELLKAKKADDEETVAELEARINVLNAELSAQEEAICSYATEMATRTLAGKFASELDLNIYGQILAEVKSVVFKNFNSDSKDVREGKRSIRTYKKGMPIPFPWNKTIRLEAVKKESSSKHDEDEYEVYLNWYKSSRTEKKAIRFRLDFGKDKSNNQQIVKRCLNLDNTSSESYQLQTSSIQMKKGSEGAELYLLLVVNIPQDQHVLNKKIVVGVDLGINVPAYVATNCTEERKSIGDREHFLNARIAFHRRFHSFQKLKGTTGGRGRKKKLEPLERLREKERNWVHTQNHLISRDVINFALQTKAATIQMEDLSGYGKDEEGNVKPENKFLQSRWSYFELQSMIKYKAAKCGIKVNLINPSYTSQTCSWCGQMGVRESTSFVCQNPECKKYGKDIHADYNAARNIARSNKTVKNE

SEQ ID NO: 90 - protein, 490 aa, unidentified
  REGION 1..490: note = uncultivated organism
  REGION 1..490: note = MG119-62 Effector
MATEYTCITRKIEVHLHKHGDGEEAENRRAEEFRMWNDINDNLYKAANRIVSHCFFNDAYEYRLKIQSPRYREIQRKLRYSKRDKLSDAEIKELKAERNELDKEFRKQYQAFMLGGSSEGYKSTIPQNSTERIVNNEFGDIIPSNVLSCLNQNVFQTYKQYRTDVEFGKRTISNFKKGMPVPFSIKANKNLMLKKREDGSIFVYFPKGLEWDLSFGRDRSNNREIVERILSGQYDVGTSSLQEAKNGKVFLLLVVKIPKQSDKLDPNRVVGIDLGINIPLYAALNDNEYGGMSIGSREQFLKMRMRMVAQKRELQRNLRHSANGGRGRSHKLQALERLEGKERNWVHLQNHIFSKNIIEFAVKNNAGVIQMERLTGFGHDRNDEVDDGFKFILRYWSFFELQSMIEYKAEAAGIEVRYIDPYHTSQTCSFCGHYEKGQRIDQATFVCKNPECEKGKGKKRSDGTYTGINADWNAARNIALSDKFVDKKKK

SEQ ID NO: 91 - protein, 473 aa, unidentified
  REGION 1..473: note = uncultivated organism
  REGION 1..473: note = MG119-63 Effector
MPIITRKIELKIVKDGLTDEEYDQQWKYLYQINNTIYQAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFVKENGIYLKWFKGILFKLEFGKDASNNRCIVERLIESDKQQKGKGEDYVANNSSIKLIKKGNDKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 92 - protein, 467 aa, unidentified
  REGION 1..467: note = uncultivated organism
  REGION 1..467: note = MG119-64 Effector
MTIITRKIELKLCTDGLSDEERKMQWKLLYRINDNLYKAANNISSKLYLDEHVSSMVRLKHAEYLLLLKEIDKANRKKEPDVDVINGLNQKLNAIEVEMSDQAKAICKYATEMATKSLAYRFAQEFDLDIFGQILSCVQQNTHATFTKDMLDVSRGERSIRTYKKGMPIPFPWNKSIRITNKGEEFYLRWYKGIRFHLFFGKDRSNNRAIVKRCLKLDTDYQGEYKICNSSIQIVKKDDGVKLFLLLVVDIPKENRKLNNRIVVGVDLGINVPAYVATNITEERKAIGDREHFLNERMAFQRRYKSLQRLKNTAGGKGRKKKLEPLEALRKAEHNWVHTQNHLFSREVVNFAVQAEAATIHMEKLKGFGKDNEGNADERKEFVLRNWSYYELQNMITYKAAKYGIKVEYIRPAYTSRTCSWCGELGFREGVTFICENPNCKKCGEKIHADYNAARNIANSKEIEDNE

SEQ ID NO: 93 - protein, 468 aa, unidentified
  REGION 1..468: note = uncultivated organism
  REGION 1..468: note = MG119-65 Effector
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVDLGINVPAYVATNITEERKAIGDREHFLNSRMAFQRRYKSLQRLRGTAGGKGRAKKLEPLERLRKAEHNWVHTQNHLFSREVVDFAVKSHAATIHMEDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 94 - protein, 383 aa, unidentified
  REGION 1..383: note = uncultivated organism
  REGION 1..383: note = MG119-66 Effector
MSTQEKAICKYTDEMSPLSLAYGFATELELDIYAQILTQIQSKVHKDFTGDSEDVRDGKRSIRSYKKGMPIPFPWNNTIRIEAVEKVKVEGKETKSRRDREDYDFYLNWYNGIKFRLNFGIDRSNNYQIVKRCLNLDENCRDNYQLKTSSIKMVNGTKEMYLLLVVDVPQEKYELNNNIVLGVDLGINVPAYVATNITEDRKAIGNREHFLNARMAISRRFRSLQRLQGTAEGRGRKKKLEPLEPMREKERNWVRTQNHLYSRDIVNFAIRVKAATIHMEDLSGYGKDSEGNVKEEKKFLLGKWSYFELQTMIKNKAQKAGIKINIVKPAYTSQTCSWCGERGLRKGTSFFCQNPECEQYANKDIHADYNAARNIARSKEIVK

SEQ ID NO: 95 - protein, 469 aa, unidentified
  REGION 1..469: note = uncultivated organism
  REGION 1..469: note = MG119-67 Effector
MPTITRKIELSLCTDGLSDEQLKEQRQLLYHINDNLYRAANNVSSKLYLDEHVSSMVRLKHADYLSLLRDLARAEKQKSPDEALISELRSKLAAAQREMTEQELAICRYATEMSTQSLSYRFVTEMETHIFAKILDCLKQGVYATFNSDARDVKRGERSIRNYKKGMPIPFAWSDSVRIEQEADEFYLRWYNGIRFRLVFGKDRSNNRLIVKRCLKLDKDYEGDYKLCNSSVQMVKREGKPKTFLLLVVKIPQEQVALNKKIVLGVDLGINYPVYAATNCTEERMYFGDREHFLNTRMQFQRRYKSLQRLKGTTGGKGRKKKLEPLERLRKAERNWVHTQNHLFSQKTVDFALQTHAATIHLEDLSGFGRDSDGSAEEKKEFVLRNWSYYELQQMITYKAAKYGIKVEKIRPAYTSQTCSWCGQRGFRQGVTFICENPECKKCGEKEQADYNAARNIAKSKDVIKDDDE

SEQ ID NO: 96 - protein, 468 aa, unidentified
  REGION 1..468: note = uncultivated organism
  REGION 1..468: note = MG119-68 Effector
MPTITRKIELTLCTQDLSDEQRKEQWGLLYHINDNLYKAANNISSKLYLDDHVSSMVRLKHADYLSLLKEQARAEKQKTPDVEKLAEVRAKLAAAEQEMSDQELAICKYATEMSTNTLAYNFAKEIETKIFGQILACLENNAHALFVDDSPNVRRGERAIRNYKKGMPIPFPWNKSIRIEAKDSEFYLRWYNGLRFLLTFGKDRSNNRMIVKRCLKLDKDYDGEYKLCNSSIQMVKREGKPKLFLLMVVNIPQEHVELNKNIVVGVDLGINVPAYAATNITEERKAIGDREHFLNTRMTFQRRYKSLQRLKTTAGGKGRTKKLEPLERLRQAEHNWVHTQNHLFSREVVNFAVQTHAATIHLEDLSGFGKDNDGNADERKEFVLRNWSFYELQAMIAYKAAKYGIKVEKIRPAYTSKTCSWCGQQGFRSGVTFICENPECKQCGEKVHADYNAARNIANSKEIVRNNE

SEQ ID NO: 97 - protein, 471 aa, unidentified
  REGION 1..471: note = uncultivated organism
  REGION 1..471: note = MG119-69 Effector
MPVITRKIELKIVKDGLTDEEYDQQWKYLYQINNTIYLAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDKQQKNKGEDYVANNSSIKLVKYGKSTRIFLLLSIDIPAKKQVLDKDVVLGVDLGIKCPLYLAINKNDNFKMQIGDIEHFHNQRTMFQKRFKSLQKLICTQGGHGRKKKLEPLEKLKEKERNWVHTQNHVYSREVIKQALKQNARTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 98 - protein, 451 aa, unidentified
  REGION 1..451: note = uncultivated organism
  REGION 1..451: note = MG119-70 Effector
MSKITRKIEIIPDVEGLTHEESNEKCYKAFYNYDRKLYKVANLLVSQLYGLDNLLSLMRLQNEEYVDSQRKLSFKSTTDTAKEEIKKRMEEIDAELMAIKKKIAPMHPQSYSYRAVNSSEYAKDMPSDILDSLKQDVYKHFNDSKKEQIRGERSLTTYKRGMPIPFNLKKKHSIVCDGGNYYLLWFEDTRFRLNFGRDRSNNRAIIDNCIKKYKLCAAAKIQLKERKLFLLITVDIPKAESVPVKGKVMGVDLGVVNPAYVAVNDGPERSRIGNGEAFQKQRDVFRRRFRELQRSQLTQSGHGRKHKTKATEILRGKERNWVQTENHRISREIVNLASRWKVETIRMESLKGFGKNQEGEVENNHKRLLGRWSYFELQKDIEYKAAMAGIKVQYVNPAYTSQTCHVCGQRGNRIERDTFICTNPECTCYNQVQDADMNAAINIAKSNDVVK

SEQ ID NO: 99 - protein, 473 aa, unidentified
  REGION 1..473: note = uncultivated organism
  REGION 1..473: note = MG119-71 Effector
MPIITRKIELKIVKDGLTDEEYDQQWKYLYQINNTIYLAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRESKRLKLFVKENGIYLKWFKGILFKLEFGKDASNNRCIVERLIESDKQQKGKGEDYVANNSSIKLIKKGNDKSTRIFLLLSIDIPAKKQVLDKDVVLGVDLGIKCPLYLAINKNDNFKMQIGDIEHFHNQRTMFQKRFKSLQKLICTQGGHGRKKKLEPLEKLKEKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 100 - protein, 463 aa, unidentified
  REGION 1..463: note = uncultivated organism
  REGION 1..463: note = MG119-72 Effector
MEQITLTRKIELFVNEEGNKKNECYDLLHYLNNNLYKIANDSVNYLHIKKNLPEFILKHNKEDYEKYVNNLNKMSEIKNFKSTEFIELKNQNRDIINKYSNNEEYKKMIYNDSKETTLVYHNINKRLPKGLSSISASLSKKIENDYKIKEKEYFLGKSTLNIYKRGLPIPFNISGKLVKDPSLFFKFDNGKYYINLFGYNFGLFFGPDRSNNKAIIEHIINDTDKYKLCDSAIQFKDKKIFLLLSFKQPLDNTIKLDDNKVLGVDLGVNAPVYLAINDNDYYKKQIGDIHHFDSHKLQYKKRYTSLIKNLEISNGGRGRKKKLSALDKLKKVEKNWTNTEQHKISNSVIKEALNNNCKYINIENLENYKENLTENKKYIMRIWGYYGIQEKIMYKAKKHNIVVRKINPAYTSQKCHKCGNIEKTARNKQIFTCLNEKCSIFGKEIHADFNGAKNISLSTEFKK

SEQ ID NO: 101 - protein, 471 aa, unidentified
  REGION 1..471: note = uncultivated organism
  REGION 1..471: note = MG119-73 Effector
MPIITRKIELKIVKDGLTDEEYDQQWKYLYQINNTIYLAANRISTHCLFNDEYEMRLKLHMPRYKEIEKELKKLDSDKKTSDKEIRDRLLNERKELDENVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKNKGEDYVANNSSIKLVKYGKSTRIFLLLSIDIPAKKQVLDKEVVLGVDLGIKCPLYLAINKNDNFKMQIGDIEHFHNQRMMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 102 - protein, 474 aa, unidentified
  REGION 1..474: note = uncultivated organism
  REGION 1..474: note = MG119-74 Effector
MPTITRKIELYIDKGNLTDDEYKAQWQYIRQIDNTLFLAANRISSHCLLNDELEIRLKLQMPEYCKIEKLLRNSKKNNLGKKEIAELKLRRKELDAIIKKQKDEFLKTSQNSTYQLVAYEFSNIPTDILTNLNNDIVGKYGKARLDIINGIKSPSTYKRGIPIPFSVNKKLPFVFYDGEYYLEWFKGNKKVATGKELCFKLHFGKENSNNCAIVERVDESEKLGKKKGEAYVVNNSSIQLVEKGSASKIFLLLSLDIPTMKRTLDSNLVMGIDLGINYPIYYATNGNAYIKGHIGDRDSFLNERMVFQRRFKDLQRLQCTQGGRGRQKKLAPLELLREKERNWVRTKNHIFSREIIKCALKVGAGTIHLERLKNIGKDKSGNIEESKKYILRNWSYHELQEMIEYKAAMEGITVKYVNPAYTSQTCSCCGNKGERISQSVFKCLCTECSEYDKEVNADYNAARNIAKSKMFIKE

SEQ ID NO: 103 - protein, 471 aa, unidentified
  REGION 1..471: note = uncultivated organism
  REGION 1..471: note = MG119-75 Effector
MPIITRKIELKIVKDGLTDEEYDQQWKYLYQINNTIYQAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKNKGEDYVANNSSIKLVKYGKSTRIFLLLSIDIPAKKQVLDKEVVLGVDLGIKCPLYLAINKNDNFKMQIGDIEHFHNQRTMFQKRFKSLQKLICTQGGHGRKKKLEPLEKLKEKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 104 - protein, 466 aa, unidentified
  REGION 1..466: note = uncultivated organism
  REGION 1..466: note = MG119-76 Effector
MQTVNNRKVMPKITRKIELLFDRSGLSEEECKEKWRFIYQINDNLYRVANRLVNQLYLADEIDDILRLSDQEYIALRKKLANKKLDEATRISLEEQMSQVMKRVNERRSAILQRPQQSFAYSVVTDSDTEGLTAKILDVLKQDVLSHYKADTKEVLKGEKSISNYKKGMPIPFAFNDSLRLYKEDGFFYLKWYNGIRFLLNFGRDASNNQLIVERCLGISKDEISYKTCSSSIQIKKKGNHSKIFLLLVVDVPVEQYAQKPNMVVGVDLGLNVPIYAASNSTLERKAIGSREAFLNQRGAFQRRFRALQRLQTTKGGRGRLHKLEPLERVREAERNWVRTQNHLFSREVINFAIDVGASTIQMEKLANFGRDAQGEVREDKKYVLRNWSYFELQNLIEYKAKRAGIKVKYINPAFTSQTCSECGQLGERDSIHFKCTNPDCPNCGKDIHADYNGARNIAKSKDYIK

SEQ ID NO: 105 - protein, 467 aa, unidentified
  REGION 1..467: note = uncultivated organism
  REGION 1..467: note = MG119-77 Effector
MPKITRKIEMTLCTEGLSDEQRKEQWGLLYHINDNLYKAANNISTKLYLDEHVSSMVRMKHADYLSLLKELAKAEKKSPDEDLIAELREKLAAAEQEMTDQELAICKYATEMSTQTLAYKFATEIEINVFGQILACLKQAAQSNFKSDAKDVKRGERAIRNYKKGMPIPFPWNDNIRIDADGDEFYLRWYNGLRFHLTFGKDKSNNRMIVKRCLKMDKDFEGEYKLCNSSIQMVKRDGKPKLFLLLVVNIPQEHVELNKNVVVGVDLGVNVPAYVATNITEERKAIGDREHFLNTRMQFQRRYKSLQRLKATAGGKGRTKKLEPLERLRKAEHNWVHTQNHLFSREVVNFAVQTHAATIHMEDLSGFGKDDDGNADEQKEFVLRNWSFYELQNMIAYKAAKYVIKVEKVKPAYTSKTCSWCGQLGFRQGVTFICENPACKQCGEKVHADYNAARNIANSKDIIKKNE

SEQ ID NO: 106 - protein, 463 aa, unidentified
  REGION 1..463: note = uncultivated organism
  REGION 1..463: note = MG119-78 Effector
MPTITRKIELHLCTEGLTDEQRKAQWGLLYHINDNLYKAANNVSSKLYLDEHVASMVRLKHDGYLSLLNEYEKAKKKADEELIAELRNKVAAAEKEMTEQELAICKYATEMSTDTLAYRFANEIEINVFGQILACLKQGIHSTFKKDAADVKRGERAIRNFKKGMPIPFPWSKSIRIENEGSDFYLRWYNGLRFRFDFGKDRSNNRMIVSRCLNLDPDFEGEYKLSNSSLQMVKRDGSTKLFLLMVVNIPQENVELNKKIVVGVDLGINSPAYVATNVTMEHQRIGRRETFLNARMAIQRRYKSLQKLQNTAGGRGRKKKLEPLERLKETERNWVRTQNHLFSRDVVQFAVKARAAIIHIEDLSGYGKDDDGNADEKKEFVLRNWSYYELQTMIKYKAAKYGIKVEKIRPAYTSRTCSWCGHEGFRKGEIFICENPECEKCGEKENADYNAARNIANSKDIVK

SEQ ID NO: 107 - protein, 475 aa, unidentified
  REGION 1..475: note = uncultivated organism
  REGION 1..475: note = MG119-79 Effector
MPIITRKIELKIVKDGLTDEEYDQQWKYLYQINNTIYLAANRISTHCLFNDEYEMRLKLHMPRYKEIEKELKKLDSDKKTSDKEIRDRLLNERKELDEDVKNKKKDFFQCSKQNSTYQLASKEFLKYIPAEILTDLNRYVQNNHNNNKKKVKSGERALSTYKKGMGIPFSIKPQSGLRLFVKEEGIYLKWFKGILFRLEFGKDTSNNRCIVERLIESDKQQKKNKGEDYVANNSSIKLIKKGNDKSTRIFLLLSIDIPAKKQVLDKEVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKHNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 108 - protein, 468 aa, unidentified
  REGION 1..468: note = uncultivated organism
  REGION 1..468: note = MG119-80 Effector
MPTITRKIELNLCTEGLADEEQKAQWNLLYHINDNLYKAANNISSKLYLDDHVSSMVKLKHAEYLTLVRALEKAQKQKTPDETVIEDLRRQVAAAEKDMTDQELAICNYATEMSTQSLSYRFATEIETNIFAQILDCLKQGVYATFNSDAKDVKRGERAIRNYKKGMPIPFPWNKSLKIEHKDEEFYLLWYNGLRFHLNFGKDRSNNRLIVQRCMKMDKDYEGDYKMCNSSIQFVKREGKPKFFLLLVVNIPQEHVELDKKIVVGVDLGINSPAYVATNVTMERQQIGRRETFLNGRMSFQRRYKSLQRLQTTAGGRGRKKKLEPLERLRNAEHNWVHTQNHLFSREVVQFAVKARAATIHMEDLSGFGKDNDGNADERKEFVLRNWSYYELQTMISYKAQKYGIKVEKIRPAYTSRTCSWCGHEGFRKGEIFICENPACEKCGEKENADYNAARNIANSKDIIKNHG

SEQ ID NO: 109 - protein, 494 aa, unidentified
  REGION 1..494: note = uncultivated organism
  REGION 1..494: note = MG119-81 Effector
MPTITRKIELKIVKDRLTDEKERLTDEKYDQQWKYLYQINNTIYQAANRISTHCLFNDEYEMRLRLHMPRYKEIDEGLENIKTELEKLNTKKKASDKEKRDRLLNEQRKLVDERKELDKDVKNKKKDFLQCSKQNSTYQLVSKEFLKYIPAEILTDLNRYVQNNHSNNKNKVKSGERALSTYKKGMGIPFSIKSESGLRLFVKEEGIYLKWFKGILFKLEFGKDSSNNRCIVERLIESYKQQKKKGEDYVANNSSIKLIKKGNDKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIAER

SEQ ID NO: 110 - protein, 454 aa, unidentified
  REGION 1..454: note = uncultivated organism
  REGION 1..454: note = MG119-83 Effector
MPTITRKIELLLDRTGLSKEEVNARWNILHQINDNLYRVANNLINKLYLTDEIEDILRLNNQEYIDLRKQLGKKGLEDEKKAELEERMQEVYAEMNQQRSKILQRPRQSFAYSAVSEDGTEIINAKILDTLKQNVLSHYNADTKEVHRGEKSISNYKKGMPIPFSFDKSVRLYERNGEFFLKWYRDIQFILFFGRDASNNQLIVERCLGISGDGITYKMCSSSIQFKGKKIFLLLVIDVPRETFTPKKDMVVGVDLGLNVPIYAATNLTPERKAIGNRETFLNQRIAFQRRFKALQRLQLTQGGRGRSHKLAPLERLREAERNWVRTQNHLFSKGVIEFAKQVGASTINMERLTHFGKDKEGEVTEEKKYVLRNWSYFELQNLIEYKAKMANIKVRYVSPAYTSQTCSECGQTGERDSIHFKCTNPECKNFGKDIHADYNGAKNIAKSTNVVKE

SEQ ID NO: 111 - protein, 450 aa, unidentified
  REGION 1..450: note = uncultivated organism
  REGION 1..450: note = MG119-84 Effector
MKVTRFQLICCTTDKDGNEIDYKDIFHLLWKLQDETRSIKNKAIQLLWEWYNFSSEYKKQYGDYPSNEDTLGYKGGIRSYLYDRLKDDSSMNTANYSCTLDQSRSQFQDGLKDYLKGDRSIIEYKNNQPLELHNKSIVLERTNDEYIVKLSILSKDGAAKYNMPQRIIFKGDVRDKSTRDILNRCISGDYKVCGSKLIYIKKKKGGGDKNKSGKWYLNLGYSFESEAKALDEDKILGIDLGIVKPFVASVYGDRSRLWVDGGEDSEIEVFRRRCEARRKALLRQRKICGDGSIGHGYKTRTAPANALEDKIARFRDTINHKYSRAIVDYAVKQNCGIIQMENLKGITDGEQPRYLKNWSYFDLKTKIENKAKEKGIKVVYIEPQYTTLRCSKCGYIDKENNPNQATFICQSCGFKANADYNASQNIAIKDIDKIIKAELKKEEKEKAKNK

SEQ ID NO: 112 - protein, 408 aa, unidentified
  REGION 1..408: note = uncultivated organism
  REGION 1..408: note = MG119-85 Effector
MITVRKIKLSIRESDEESRKQKYKFIRDAQYAQYRALNLAMGVLSSEYLKSNKDTNKEDYKNAIKSLTNSNPIFSSIQFGRGVDTLSLVTQRVKKDFKISIKNGLARGERNITSYRRTHPLMTRGRSLRFYYEGDDIVIRWVNKIEFEVVTGSNKMKENIVELRRTLHKVINKEYDVKESSLMFDKNNNLILNLTLDIPNELSYQPIEGRTLGVDLGIATPAYVCLSDDTYVRKGIGSIEDFLKVRTQLQKRRRVLNQSLVMAKGGKGRKKKLKALESFKEKERNFAKTYNHQLSHEIVKFAKNHKCEFINLEKLTKEGFNNRILRNWSYYELQSMIEYKAEREGIKVRYINPAYTSQKCSKCGHVDKANRQTQSRFKCVECDFELHADHNASINIARSEEFVQGTGS

SEQ ID NO: 113 - protein, 449 aa, unidentified
  REGION 1..449: note = uncultivated organism
  REGION 1..449: note = MG119-86 Effector
MTTKVIKYEIIKPIDIDWKLAGEIFNKLQYQSYLLANKTIQMFWDFQNLSFTYKERFGKYLNMKDIPNKYTSIEGDISSQLRNQFINLPSGTFDSVVIKAKNKWNQHKLEILKGSESMVNFKRSFPICLRDRQIKIIKNDENTYILRLSLLSKEYKTELELKKGQIDFLIKAYDNTQKVILERLINNEYKIGASEIFKIKKGKTKWFINITYTFQQEENLNLDKNNIMGIDLGVCNPAYIAFNNSLNRYKIEGNEIIEFRRRIEKRRNELLKQGKYCGEGRIGHGRNTRIKPIEKLRNKVENFKDTTNHKYSKYIIDLALKNNCGVIKMEDLSGIAEGEKKATFLGMWSYYDLQTKIEYKAKEIGIEVIKINPKYTSARCNKCGYIHRSENKDIWRPKQDTFKCMNCDYGHKYFVHADYNAAKNISTPDIEEIINKQLKLQEVRVKFAE

SEQ ID NO: 114 - protein, 438 aa, unidentified
  REGION 1..438: note = uncultivated organism
  REGION 1..438: note = MG119-87 Effector
MNTTKVSRYQLLFDDENNELTFKQSNEVLFQLQKEMMQMANRMVQLYWEFSGYQSDYKKQFDAYPDKEKLLEDLKYSNIRSYIYDVLSKEFNKNNTGNVATLQQLVDKRYKQNRKEVQQGKRSIDSYKATIPISLHKGSIRLLKENGDYYAWLSLLSNEYKKELGIKKGRMRFKLLMGKSYSSMQILDSILSGEFHHTSSSLGYRNGKWFLSLGYQFEAKEPKLRESRVMGIDLGVVKPLVIAYNDMPLHHEIDSNEIQVFRDQMHARRRALGRQTKYCAESNIGHGVKTRTKALEKLKQAEANFRDRINHQYSRYVVNLAVKYGCKTIQMEDLTGISKKDTFLKNWPYYDLQTKIEYKARENGIDVVKINPRFTSQRCSKCGFISPENRPDQATFQCKKCGFGINADRNAATNIAQPGIEKIIQETIKQMSAKGKSS

SEQ ID NO: 115 - protein, 431 aa, unidentified
  REGION 1..431: note = uncultivated organism
  REGION 1..431: note = MG119-88 Effector
MNTTKVSRYQLLFDDEHNELSFQQANEVLFQLQREMMQIANRTVQLYWEFSGYQSDYKKRFDAYPDKEQLLEDLKYSNLRSYTYAVIAKEFYKNNTGNVSLLQQNVDKRYKQNRKDVQMGKRSIDSYKATIPIALQRKSILISKEGANFCVRISLLSNAYRKELGVKNGTLSFKLLFGKSYSAEQILEDIFAGKFKHTSSSISYRKGKWFLNLGYNFESEEPELRPSRVMGIDLGVVKPLVIAYNDTPVHQEIDSNEIQAFRNQMLARRRALGRQTKHCAESNIGHGVKTRTKALEKLKQTEANFRDRINHQYSRYVVDLAVKYKCKTIQMEDLSGISKDDIFLKNWPYYDLQTKIEYKAKEKGIDVVKINPKFTSQRCSKCGYISPENRPDQATFRCKKCSFTLNADWNAALNIAQPGIEEIIRETIKQK

SEQ ID NO: 116 - protein, 442 aa, unidentified
  REGION 1..442: note = uncultivated organism
  REGION 1..442: note = MG119-89 Effector
MTTKVMKYEIIKPINIDWELAGEIFNKLQYQSYLLANKTIQMFWDFQNLSFTYKERFGKYLNMKDIPNKYNSVEGDISNQLRNQFINLPSGTFDSVVIKAKQKWNQHKLEILKGTESMINFKRSFPICLRDRQIKIIKNDENTYILRLSLLSKEYKTELELKKGQIDFLIKAYDNTQKVILERLLNNEYKIGASEIFKIKKGKTKWFINITYSFEQENKINLDKNNMMGIDLGVCNPAYMAFNNSLDRYKIEGSEVIEFRRRIEKRRNELLKQGKYCGEGRCGHGRTTKLKPIEKLRGKVENFKNTTNHKYSKYIIDLALKNNCGIIQMEDLTGIADGEKKATFLGGWTYYDLQTKIEYKAKELGIEVIKIKPKNTSARCNKCGHIHRNEDKDIWRPKQDTFKCMNCDYGHKFFVHADYNAAKNISTLGIEKIIEKQLKNKK

SEQ ID NO: 117 - protein, 431 aa, unidentified
  REGION 1..431: note = uncultivated organism
  REGION 1..431: note = MG119-90 Effector
MNTTKVSRYQLLFDDEHNELSFQQANEVLFQLQREMMQIANRTVQLYWEFSGYQSDYKKRFDAYPDKEQLLEDLKYSNLRSYTYAVIAKEFYKNNTGNVATLLQNVDKRYKQNQKDVRLGKRSIDSYKATIPISLHRKSIQISKSKEDYDIRISLLSNVYRKELGVKSGLIYFKLITGKSYSSKQILESILSGKFHYTASSISYRKGKWFLNLGYNFESEEPELRPSRVMGIDLGVVKPLVIAYNDTPVHQEIDSNEIQAFRDQMLARRRALGRQTKHCAESNIGHGVKTRTKALEKLKQTEANFRDRINHQYSRYVVDLAVKYKCKTIQMEDLSGISKDDIFLKNWPYYDLQTKIEYKAKEKGIDVVKINPKFTSQRCSKCGYISPENRPDQATFQCKKCGFSLNADWNAARNIAQPGIEEIIRETIKQK

SEQ ID NO: 118 - protein, 444 aa, unidentified
  REGION 1..444: note = uncultivated organism
  REGION 1..444: note = MG119-91 Effector
MRIAYLIQQKPNLIQSGEIMTKVTRIYLISKQENVDYKKVCSVLWDLQRQTREIKNKTIQLCWEWLGFSSDYNKRTGEYPKEKDVLNYTLDGFIYDHFKKGYDLYSSNASCVTREASSKFKNAKAEILRGDRSILSYKSNQPLELHNKAISIYNENDKFYVKLKLLNKAGKEKHGVDGDFLFEMKVKEKSIRTIVERCIDGIYKISGSKLIYDNKKKMWCLNLSYSFKSTENKALDKDKILGVDLGVVHPICASVHGDLNRLIIKGGEIEIFRKRVEARRLQLLEQAKYCGDGRIGHGRHTRTLPAFKVEHKIEQFRNTANHKYSRALIDFAVKNGCGTIQMKNLKGVTTDNLFLKNWSYYDLQTKIEYKANEAGIKVVYIEPQFTSQRCSKCGFIHTENRPEQAVFKCQKCGYSANADFNASQNISIKQIDKIISETISANME

SEQ ID NO: 119 - protein, 426 aa, unidentified
  REGION 1..426: note = uncultivated organism
  REGION 1..426: note = MG119-92 Effector
MGKTDNKEEKIFRASNKVKITIVEDDPDERNRKYKLIHDEMYKQYLALNMCMSLLATQNTLRMYNSGAENRLLSQIAKLDKKISEKQQIADKDANTEKEQTRKQKAIEDIAKLEEEKSKLREEFDKKAANRTDIDVKFHEQYTSDLYQIIRNEFDFASSATIASVVRKVKDDFKNSFSMGYAKGERSLISYKRSFPLMIRGNEDACYDNSKRKWLDFYYDGDDILIRWLDGIIFKAILGNNPNRNMEIKHTLHKIITGEYRVSQSSIEFDDKGHMYMNVNYRFKKNSKTEIIPGRTLGVDLGIATPAYVCLSDTTYVRSGIGSANDFFRIRQQMKSRRKSLYKSLSLVKGGKGRKKKLQAINSLRDKERNFAKTYNHQLSNKIVKFAKDNGCEFINLEHLTKNGFEDAILSSWSYYELQNMIEYKA

SEQ ID NO: 120 - protein, 452 aa, unidentified
  REGION 1..452: note = uncultivated organism
  REGION 1..452: note = MG119-93 Effector
MSVKLTKVMKYQIIKPIDIEWELVGKIFNELQYQSWKLANKTIQLFWDFQNLNFTYKERFGEYLKIKDLPNKYTSIEGDISSQLRNQLINLHSGTFDSVIIKAKDKWNQHKQKILKGEESIVNFKKNFPICLRNRQIQIIKNDNNTYTLRLSLLSNEYKKELELKTGQIDFVIKAHDNTQKVILERLINKEYKINASEIFKIKKGKTKWFINLTYSFEQDDKINLDKNNIMGVDLGICTSAYMAFNNSQNRYYIGGSEVIEFRRRVEKRRNELLKQGKYCGEGRIGHGRTTRIKPIEKLRGRVENFKNTTNHKYSKYIINTALKNNCGIIQMEDLTGIAGGERKATFLGMWSYYDLQTKIEYKAKESGIEIKKIKPKHTSARCNKCGHIHKSEDKDIWRPKQDTFKCMNCDYGHKFFIHADYNAAKNIATPGIEEIINKQLKLQEVRVKFAE

SEQ ID NO: 121 - protein, 448 aa, unidentified
  REGION 1..448: note = uncultivated organism
  REGION 1..448: note = MG119-94 Effector
MSVKLTKVMKYQIIKPIDIEWELAGKVFNELQYQSWKLANKTIQLFWDFEKLSYSYKQRFGEYLNIKDLPNKYNSVKGDIRNQLKNQFINLPSDTLDSVLGEVESKWCQHKSKIFKGEEEMINFKKGFPINLSSKQIKIIKNDDNTYIFRLSLLSRAYKSELELKKSQIDFLVIAHDNTQKVILERILNNDYKIGVSKIFKIKKGKTKWFINLTYSFEQDNQINLDKNNIMGIDLGVCNPAYMAFNNSHNRYHIGGSEVIEFRRRVERRRNELLKQGKYCGEGRIGHGRTTRIKPIEKLRGRVENFKNTTNHKYSKYIIDTALKNNCGTIQMEDLTGIAGGERKATFLGMWSYYDLQTKIEYKAKELGIEIKKIKPKHTSARCNKCGHIHRSEDKDIWRPKQDTFKCMNCDYGHKYFIHADYNAAKNIATLDIENIIGEQMKRQNINT

SEQ ID NO: 122 - protein, 419 aa, unidentified
  REGION 1..419: note = uncultivated organism
  REGION 1..419: note = MG119-95 Effector
MTKIVKLQIIKPINQDWKLFGDILFNLRNDTRQIKNKAIQECWEYQGFSSEYKDKFEEYPKPKDVLNYSGLSGYIYNKYKLKFNRNNKANYTTIINEAVDMWKSDIKDVLRGDKSIRNYKSDLPLDLHNKSINLIKESNEYYLDLILIFNKYKKELEFKSGKFLVLIKVNDNNVKSTLDKILNNEYKISASKLIRKKNKWFINLCFSFETIASSSLSVDNIMGVDMGIIYPAYMAFNNSLDRYKIEGYEIENFRKTIESRKNKLYAQGKYCSGGRIGHGIKTRIKPIEFATKRVANFRDTTNHKYSKYIINMALKHNCGTIQIEDLKHISEDNAFLKKWSYYDLQNKITYKAKEKGIKVIKINPRYTSQRCSQCGNIDKDNRKTQKDFLCLICGFKTNADYNASRNIATKDIEDIIENS

SEQ ID NO: 123 - protein, 434 aa, unidentified
  REGION 1..434: note = uncultivated organism
  REGION 1..434: note = MG119-96 Effector
MKFSKVVKIALVSVQKDKEGNEMDYKTICQILWELQRETREIRNKSIQYCWEFSNFSSDYKKEYLEYPKPKDVLNYASIDGYVYDRLKENSSLHTNNFSTSTRDAIKKFKNLTKDILSGKCSIPEYKNNQPLDLHNKSIGIDKIKDCYYITLKLLNKKGKEKYDFGSSSIKFKALVKDNSTKTIISRLLNGEYKIGASKLIYNQKKKQWFLNLTYNFESEVTTELDEDKILGVDLGINCPFCASVYGSKKRLMIPKDEIEHFRNTVEVRRKALLKQGPFCGEGRVGHGRNTRNKPAYDIGNKVARFRDTVNHKYSRELVNYAVKNHCGVIQMEDLTGINKDSDKTFLRNWSYYDLQTKIEYKAKEAGIKVKYINPMYTTQRCCKCGHIESDNVKKHQRFACSKCGYTENADYNASQNIAMPDVEEKVAESLKVS

SEQ ID NO: 124 - protein, 439 aa, unidentified
  REGION 1..439: note = uncultivated organism
  REGION 1..439: note = MG119-97 Effector
MNKIMKYEIIKPVDCDWDLFGNILRKLKNQTRNLLNKTIQFMWEYDNFSADYKKIHNVYPNSLDILGYKGSQAVQNYAKMKLKKEWDFLFNSSNVDATLATALTRWKTDKVDILKGNKSIPSFNKNNTIDLRNSSITVIKENNSYYLNLSLISNKYKKELGRSGQFLTLIKVKDKTQKAILDRILNGEYKVSASKIIYLEKKKKWMINLGYGFEPKKQNLNKDNIMGIDMGIVYPIYIAFNNSLKRYKIEGGEIDRFRKQTEKRKNQLYRQGKYCGDGRVGHGIKTRIKPIEFTQEKVANFRNTTNHKYSKYIIDLAIKNNCGIIQMEDLSGISTNNKFLKNWSYYDLQQKIEYKAKEKGIEIKKIKPQYTSQRCSKCGYIHKDNRPKGDKGQSYFKCLECEFKTNADYNAAKNISIKDIDRIIESKCKELDITYNKNN

SEQ ID NO: 125 - protein, 429 aa, unidentified
  REGION 1..429: note = uncultivated organism
  REGION 1..429: note = MG119-98 Effector
MVKVVKIYLISEQVDEQGKDVDYNTICGVLWDLQWETREIKNKTVQLCWEWSGFSSDYYKKYGEYPKEKNLLDYTMGGFVYDKLKSKYHLYTANLSTTSQNTCGIFRTYKVDFVKGNRSVLSFKADQPLDVHKKSISIDRIDDNYFVKLKLLNKSGIQKYGIRDDFHFRMLVKDNSTKTILERCVGGDYKAAASKIIYDKKKKMWCLNLSYEFDVNTAKDLNKNRILGIDIGIVYPVVASVNGELDRFVIQGGEIETFRRRVENRKKSLLKQTKYCGDGRIGHGRNKRTEPVDIISDQIARFRNTANHKYSRAVIDYAVRKQCGTIQMENLKGITDKSDRFLKNWSYYDLQQKIEYKAKEKGINVVFINPKYTSQRCSRCGYIDSANRPKLPNQSKFLCIKCGFTENADYNASQNIALYNIEKLIDAEA

SEQ ID NO: 126 - protein, 437 aa, unidentified
  REGION 1..437: note = uncultivated organism
  REGION 1..437: note = MG119-99 Effector
MSNILSMPVKVMRYQIVKPVDADWELLGKVLRDLQDDTRKVLNKVIQLCWEWQGFSAEYKQQYGIYPKTSDFSHYKNIDGYCYKKISDFFTRLHTGNLSMTIKRGVQRWKTDLAEVIKGSKSIASFRSNCPIDIHNKSINIFNVEKEYYAKLSLLSKTYKKELSRDLGQITVLINPGDKSGKDILDRCISGEYKISSSQILQKKDTNTSKNNKWLLNLSYSFKSQKKIVDSNKVLGIDMGIIFPVYMAIHGTLIRDKIDGGEIEHFRKEVENRKIKLQRQGKYCGQGRIGHGTKTRVRPIEFAKEKIANFRNTVNHKYSRYIVDFALRNRCGIIQMEKLEGINKDKTFLKSWPYYDLQQKIIYKANEAGITGKFIDPRYTSQRCSKCGYIDEENRPEQKTFICKSCGFETNADYNAALNIANPDIENLIKKELSREA

SEQ ID NO: 127 - protein, 434 aa, unidentified
  REGION 1..434: note = uncultivated organism
  REGION 1..434: note = MG119-100 Effector
MTKVVRVYLIIEQKDKNGDIVEYTKINKLLWDLQKQTRIIKNKAIQYYWEYKNFSRDYCEKNGNYPSVEEILTYKTVDGYINNRLKIDNDLCAINRSSTIKHAIAEYKNAESDIKDGTRSIINYKSDQPLDLHNTSIHIEYIKEEYYLFANMVSREYAKANNFENARIGFKLMLQNNKSAQTIIDRCLNGDYKISESKIIYDRKKKQWCINLAYSFSPSNVQQLDYNKILGVDLGITYPLCASVYGEYNRLAIHKGEIENFRNKVEARRYSMLRQGKNCGDGRIGHGIKCRNKPAYNIEDKIARFRDTTNHKYSRALIEYAIKNNCGTIQMENLAGITDKAERFLKNWTYYDLQTKIQYKAKECGIKIQIINPQYTSQRCSRCGYIHSDNRKTQANFLCLKCGFAANADYNASQNISIKDIDKIIKKELENMRT

SEQ ID NO: 128 - protein, 433 aa, unidentified
  REGION 1..433: note = uncultivated organism
  REGION 1..433: note = MG119-101 Effector
RYQIIKPLDCDWKQFGQILRDINYETRQILNKTIQFAWEYEGFSSDYKKEFDKYPKTKDFLNYASVHGYAYNKLKTEYIKLNSGNLAQTIKRATDKFKSDRLDILKGNKSIPHYKKDCPMDVVKGSIQLYYEDKSYMANLSLISNTFKKELERKSGQFLVLLKVGDNTQKVILDRIINGEYSLSASQIINRKNKWFLNLTYQFETEKKELNNNRILGIDLGIVNVATMQIYDAEKNKYDWLKYNQCVLDGKELIHFRQKIEARKRSLQKQSKVAGGGRVGHGYNTRMKHLLTSNDKIAKFRDTYNHKVSKYIIEFALKNECGIIQMENLAGFKGEGCNLLENWSYYDLQNKIQYKAKEYGIETVFINPKCTSQRCHVCGNIDKNNRDIKKNQAKFKCTKCGYEGNADINAARNISIQHIEEIIADYIENNKVS

SEQ ID NO: 129 - protein, 447 aa, unidentified
  REGION 1..447: note = uncultivated organism
  REGION 1..447: note = MG119-102 Effector
MIGCVEVVKSLVKVVKMYLKSEQMNEQDEKIDYRTINSMLWDLQKETRDIKNKTVQLCWEWNNFSSDYYKKYGEYPKEKDILNYSMGGFVYDKLKSGYNLYSANLTMSSRETIKQFNSYKKDYITGVRSISSFKSNQPLDVHNDAIRLCRDNDDFYVYLSLLNKMGAQKYGIKGSFRFKILVRDNSTYTILDRCINGDYKVAASKLIYDKKKKMWCLNLSYKFETKKENNLDKNKILGIDMGIAAPIVASVYGDLDRFVIHGGEIEAFRRRTEARRRSLLQQTKYCGDGRVGHGVGKRMEPANNIKDKIARFRNTTNHKYSRGVVAYAIKKGCGIIQMEDLKGISDKADKFLKDWSYYDLQQKIEYKAKEASIVVSYVNPQYTSQRCSKCGYIDSQNRPKEPDQSTFKCQNCGYSANADYNASQNIAIRNIEKEISKTIENNSANNK

SEQ ID NO: 130 - protein, 450 aa, unidentified
  REGION 1..450: note = uncultivated organism
  REGION 1..450: note = MG119-103 Effector
MSITCKIKGENMGIKNTKVMRFQIIKPLDCEWDLLGQVLREIQYETRQILNKSIQLSWEYNGFSNDYKKIHGDYPKSKDILNYSNIFGYAYNKLKQEYIKLNTGNLSQTIKRGTDKFKSDMADILKGNKSIPNFKKNCPIDLKSKSIQIYKEEDSYMANISLISNNYKKELERNSGQFLVLLKAGDGTQKAILDKILNGEYKKCASQIIRHKNKWFLNLTYQFVSEKKKLNSNKILGIDLGIVNVVAMQIYNAEKDKYEWIKYNQAMISGEELIHFRQKMEARRRSLSKQSKVAGKGRVGRGYKTRMKPLLNISDKIARFRDTYNHKISRYIVDFALRNECGIIQMEDLAGFKGKGNSLLENWSYFDLQEKVEYKASEHGIEVKFVNPKYTSQRCSRCGYINEENRKRQDRFRCVECDYEANADINAARNIAIPNIDDIIKESLTIEITA

SEQ ID NO: 131 - protein, 478 aa, unidentified
  REGION 1..478: note = uncultivated organism
  REGION 1..478: note = MG119-104 Effector
MSKKEIRSVQKIKVTIVGDTPEEREAKFKIVRDEMYNQWRALNLCMSLLATQNTLQQYNSGAENRLKAQLKKLDANIDKQQKIIDNPKTKEDKLAKAIAMIKTLNDEKTKLEQDYKDKESYRTDIDVKFNEMYVKELYQIITKEFDFFASNTASCVVQKVKNDFGNSLAEYSRGERSLINYKRSFPLMLRGNADSEYESGTLKGLKFYYKDEGIYIRWCGGIEFKVVEGCNLERNLQYKHTLHKLANGEYKVSQSSLQFDKNNHLILNCNYRYNKEVDVDCIENRVLGVDLGIAIPAYVCLSDDSFIRQGFGSVNDFVRVREQFSKRRRVLQQSLTMVKGGRGRKKKLKALDRFESKERNFAKTYNHQLSAKIVKFAKENKCQFIHLEKLTKEGFNSKLLGKWSYYQLQQMIVYKAERVGIGVRFVNPAYTSQKCSKCGHIDKENRKTQAKFECIKCGLRLNADHNASINIARSDEIV

SEQ ID NO: 132 - protein, 443 aa, unidentified
  REGION 1..443: note = uncultivated organism
  REGION 1..443: note = MG119-105 Effector
MVTKVLRIEIKRVINTDKSMFNKVMHDVRYQCWKFANKAITMLWDYQQFDFSYKERFGEYFLKEQDKLPNGYKWVGSDILNVLKKDISMLESNCKDAMIRMVEQKWNNDLKDVLTGNKSIATFKRSLPVELHNKQFMDSKKNVRIFSENGVYSTVILLLSKRVAREMGMSDGKFHLELLVKDNYQKAIVDRVINGEYKLSIIKMQYNEDKKKWFLLLAYTFEAQKKQLDENRVLGIDLGLKIPAMLAISDNPFFCRVIGNSDEIKMFEKQVVNRKKRLQKARVWASEGSIGHGTKTRIKPLNNIGKKISNYKKTMNNCWSREIINVALRENCGTIHMENLRGISGRSKFLKSWTYYQLQQMIINKASEHGIKVIKIDPSFTSQRCYECGYINKGNRKTQEEFKCVICGYKCNADLNAARNIAIKDIDKVILNQLELQKNNLNI

SEQ ID NO: 133 - protein, 404 aa, unidentified
  REGION 1..404: note = uncultivated organism
  REGION 1..404: note = MG119-106 Effector
MKTVRKLKLTIIGNEEERNAGYQFIRDSQYAQYQALNISVAYLGALYLEYKDFNNEEYKSRKRNFKFNKSNPKFEGISFGKGVDTLSLISQYVKRELSNTPLKQLARGERSLTTFKRTFPLMVTGRSLKFNYDKENNVRIKWVNKISFKVVLGSGKIKENKIELQHTLNKIINGEYKVGQSSLYFDKNNNLILNLTLDIPEKDTDEVVKDRVLGVDLGIKYPAYMCLNDDTYKRESLGCAEDFIRVREQMQYRRKRLQHSLKLTNGGKGRNKKLQALNRLRDKEKDFATTYNHAISKKIVEFAKKHKCEYIHLEKLTKDGFPNTILRNWSYYQLQSYVEYKAKREGIKVRFVDPAYTSQTCSRCGHIDKDNRQTQEDFKCIKCGFELNADHNAAINIARSNNFI

SEQ ID NO: 134 - protein, 437 aa, unidentified
  REGION 1..437: note = uncultivated organism
  REGION 1..437: note = MG119-107 Effector
MKISKIVKLRLICDQTDKDGNPVEYKTICQILFDLQRQTRELKNKTIQLCWEWNQFASDYYKKHEEYPKDKEILNYSLSGYIDNKLKIDNDLYSGNCSTTIRTACKEFNNAKLEILKGQKSIISYKQNQPLEIHNKAISLEYSDNHEWFINLALLKTDAVRKYNFSSSKMRFKIIVKDNSTKTILERCLDGIYHISASELMYSQKNKAWFLNLTYSFETSNETKLDEEKILGIDLGVACPVCASVYGSKARLKIPPNEIEAFRRKTEQRKIALLRQGKFCGDGRIGHGRKKRTQPVDKIGNSIARFRDTVNHKYSREVINFAIKMNCGVIQMENLTGITENANRFLKNWTYYDLQTKIKNKAQEAGIKVVLINPEYTSQRCSKCGYIDSENRKTQAQFICLKCGFEENADYNASQNIALKDIEKIIKKTKSKSANGK

SEQ ID NO: 135 - protein, 455 aa, unidentified
  REGION 1..455: note = uncultivated organism
  REGION 1..455: note = MG119-108 Effector
MTTKVVKIEIIKPINDTWDVLGQVLRDIQYQTWKLANKAIQMFWDFSNFNYSYQQRFGESLLGKGKSLPNGYKNVGSDILNECKEFTDKLNSNSKDAIIKMVTDKWNADFNDIINGRKSIANFKRDLPIELHNKQFMNSKKQLMLYVENGDYYIEINLISKQYAKELGKKSTAFKLLLAVKDNYQKAIVDRLISGEYKLGMSKITKTKKGKTKWYLNLVYSFTEKKDDSLDLNKVMGVDLGVNIPAALAIYGDKYFKEFVGDRDEIENFRKQIEARKRKLQRQGKWCGEGRIGHGIKTRIKPLEKLAGKIANFKDTKNHCWSRYIVDVAVKNKCGVIQMEDLSGITEENTFLKNWTYDDLQQKIKYKAEEKGIKVIYIDPAYTSARCSKCGHIHRSYEKKDWRPSQDQFICQVCGHKENADINAARNIAIPNIDKIIKEQLEKQEREQRNQKYVG

SEQ ID NO: 136 - protein, 434 aa, unidentified
  REGION 1..434: note = uncultivated organism
  REGION 1..434: note = MG119-109 Effector
MVKVVKIYLISEQTDKQGNVIDYKDIFKCLWDLQRETREIKNKAVQLCWEWSGYSSEYNRVHGIYPKEKEVLNYTLGGFLYDNLKNGYNLYSGNLSSTLQLVEKAYKASKLDFMRGDKSILSYKANQPLDIHKKAMRLEKTGNEFLLYLSILNKAGIQKYGIDSAFKFKLIIKDNATLSIVERCVDGTYQVATSKLIYDKKKKMWCINLSYNSECKHNEVVDKNRILGIDIGVACPLVASVYDDWDRCTILGGEIEEFRRRVEARKKSISKQTKYCGDGRIGHGINKRTEAVNKIGDKIACFRNTANHKYSRTVVDFAVKKQCGTIQMEDLQGITDETDRFLKNWPYYDLQTKIENKAKEEGIKVVYISPQYTSQRCSKCGYISKENRPEQAKFECKECGFKENADYNASQNIAIKNIDKIIEKDMSKDGAKGK

SEQ ID NO: 137 - protein, 426 aa, unidentified
  REGION 1..426: note = uncultivated organism
  REGION 1..426: note = MG119-110 Effector
MVKVVRLRIIKPIEADWDMLGKVLRECSYEAMLLRNRAMREYYLKNAEAYAYYKQTGKNMEYVLNDKTYTSLESYIYALFTDTCEYSNSELKSSVLRDVAKKWKGVSKDVTTGKMSLPTYKQGCPIEISGRAMTLEYDSETNNFYLKLRLLSKEASKKLELKNGVFKVLVAVEDNSKRTILERCVDGVYSLAGSKIMQVKKKWVINLSYSFEPENNSTLDKDTILGVDMGIVYPVYMAVNNSLAREKIVGGEIEAFRKRVVKRKNELYQQSKYCGDGRINHGTKKRIEPIVKIGAKEADARDTYNHKYSRFVVQTALKHGCGTIQMEDLSGITVDTDSKFLKNWTYYDLQNKIENKAKEVGIAVVKVEPAYTSQRCHKCGFISADNRKTQSDFECLECGYKGNADYNAAKNIATKDIAKIIKETKK

SEQ ID NO: 138 - protein, 464 aa, unidentified
  REGION 1..464: note = uncultivated organism
  REGION 1..464: note = MG119-111 Effector
MKTTRKLKLTIIGDEETRKEQYKIIREEQYQQYKALNLCMTLLNTHNILNSYNTGAENKLNAQIDSIDKKIEQAKKELEKKGLKESKVSKLKETIEFLENDREKLKDEYLNSSKFRSDIDEKMKEMYIKDMYTVVQNQVNFRARDMMSLVTQRARKDFKNSLKNGMAKGERSLTNYKRDFPLMTRGERWLKFEYDKDSDDILINWIHGIKFKVLLGYKKNENSIELRHTLHKVINKEYKICDSSMQFDRNNNLILNLTLDIPDKQNNNYIEKRTLGVDLGIKYPAYVCLNDDTYIRSHIGESLELLKQREQFKDRRKRLQQQLKNVKGGKGRNKKLSALNKLSDNERNFARTYNHMISKRIVEFAKKHRCEFINLEKLTKDGFDNNILSNWSYYELQNMIEYKAKREGIEVRYIDPAYTSQKCSRCGYIDKENRQTQEKFKCLKCEFEINADHNAAINIARALD

SEQ ID NO: 139 - protein, 424 aa, unidentified
  REGION 1..424: note = uncultivated organism
  REGION 1..424: note = MG119-112 Effector
MIKQIKLEISGSKELSCKELYKLLFDLRHETFLFKNRDLQCLFDFTQFEFSFRQKYGEHYKTLHDKTPAGVTPNTEAYREGKRLVSQGKNNYLKSNSNSFDAAHRSVVRKYNSELNDVLSGKRTFSTYKKTTPIELHNKQFYTGNTKTHRFYVENNKHYIAINLVSNEYSKVLNLNGTVVTLEVLVKRNHTYSIFQRILNYEYRLAMSQIKYDEKSKKWFLILAYKFDQIKNNDLVNHRILGVDLGINIPVMLAINDNPYYRKSIGLREEIEQFNKQMIKRKKILQRTARYAGRGKVGQGRKKRTEAMKKIGHNIENFRNTKNHSYAREVVNEAIKNKCGVIQIENLKGILKLNAFLKDWTYYDLMKKIKSKAEEVGIKVILINPQYTSSRCSSCGTIHFNKSKSMYRPEQDRFICQNPSCNFS

SEQ ID NO: 140 - protein, 439 aa, unidentified
  REGION 1..439: note = uncultivated organism
  REGION 1..439: note = MG119-113 Effector
MITKTMKYEIKYNKELYSLLNDIQYVIYRLKNKATSMAWDWQQFSFGYNERFGEYPKEKDIMGKTLSPDIYNVIKDEFGDFVSSSTVDVAIQDAVKKFKNDSKEINRGERSIANYKRDGAFPLRKTQVKKLEKVNSKTFTAKFSLLSNEGKKQRELKSGQIDVTLKTGGGAAVILERVMNGEYKMCDSQIGKTKNKFYFIMTYQLEKDDIVILDENKIMGIDVGYNIPAVIATNFDDWYRQFVGSGKEIRDFETKTLARKRRIQQSRKWCGKGSRGHGTKTRVKAVTKINDSVSNFKQTKNHNWSKYIVDEALKMGCGTIQMEDLSGINEKNTFLKTWSYYQLQQYIEYKAKSYGIKVVKIKPNHTSARCNRCGYIHSGKDADKWRPASENKSMFYCQNCNHKSNADHNAAKNIAMKDIEEIIKHQLYIQENVKEKLKV

SEQ ID NO: 141 - protein, 467 aa, unidentified
  REGION 1..467: note = uncultivated organism
  REGION 1..467: note = MG119-114 Effector
MITVRKLKLTIVGDEETRNQQYKLIRDEQYQQYRALNLCMSLLSTYNILNNWNSGAENKLNSQIEKLNKKVEKNKNDLKKDNLKENRIKKINESIQTLTKEKEKLQQEYLSSSEYRSDIDKKVKEMYIDDLYTVVQSQVNFKSKDMMSLVTQRAKKDFTTALKNGMAKGERSLTNYKRDFPLMTRGERWLKFEYDEDSDDIYINWLHGIRFKVVLGYKKNENSIELRHTLHKVINKEYKICDSSMQFDRNNNLILNLTLDIPLNIKNEHIEGRTLGVDLGIKYPAYVCLSDDTYKRKSIGCAEDFIKFREQIRARRYRLQKQLSMVKGGKGRNKKLQALDRIKDKERNFVKTYNHMISKNVVEFAKNHKCESINLEKLTKYGFPNMILSKWSYYELQNMIEYKAEREGISVKYVDPAYTSQTCSKCGHVDKENRTSQEKFKCIECGFELNADHNAAINIARSNNYVK

SEQ ID NO: 142 - protein, 468 aa, unidentified
  REGION 1..468: note = uncultivated organism
  REGION 1..468: note = MG119-115 Effector
MTTTKKKEKENNNNNNAPKIPTQTVRFGLLFEENAEFSYKDLRSSLWSIQRDIREFKNSAASQYFDYMTRREKIKKETGKYPADADLLNGRKIRNHLYHLAADIIPSLQTGNTTAVSESAYSRFSTDYQDVLAGRKSLASFKQDQPINIKKESIKFISTGGKLYMKLSVFSTEGAKKLGLAGGQVTFEVWRKSKSGHAIINRCISGEYSYGSGMLIYNKDKRMWECNLTYTFAPKKNDLNPDTICGIDIGFIHPIYAAVSDSRERFWLPGSEIDKFRRGVEARKRDFSRSRKFAGEGSVGHGYAKRMEPVNDIKRKIANFRETKNHQYSKKFVEFAVKNRCGVIQIRDLSGITDKTKEAFLKNWSYDDLKDKITYKAKAEGITVISDILPHYTSQRCFKCGHISKDNTNKRKFVCLSCGHEEHVDYNAAQNIAIKGIDEIIERDCIAQGIPYPPENKKEKEMEKEEDD

SEQ ID NO: 143 - protein, 467 aa, unidentified
  REGION 1..467: note = uncultivated organism
  REGION 1..467: note = MG119-116 Effector
MITTRALKLTIVGDEETRNKQYKYIRDEQYEQYKALNLCMSLLNTHYILNSYNSGAENKLKSQLDKLEKNIEKNNLELAKDNIKESKKEKLINQNIKYKEDLKKLQEEFNKASTYRSNIDIDINDMYIKDLYNVVQNQVSFKNKDLMSLVTQKAKKDFKTCLKNGLARGERSLTNYKRSYPLMTRGNRYLKFRYDEESDDIYIDWIQGIIFKVVLGHRKNENTIELRHTLHKVISGEYKICDSTMNFDKANNLILNLVIDIPINENNNYIEGRTLGVDLGIKYPAYICLSDDTYKRMAIGCAEDFIRVREQIRGRRFRLQKQLKMVKGGKGRDKKLNALDRIRDKERNFVNTYNHMVSKNIVDFAFKNRCQYIHLENLTKDGFENAILSKWSYYELKKMIEYKAERKGIKVRYVNPAFTSQTCSKCGHVDKGNRQTQEKFKCTKCGFELNADHNASINIARSKDIIK

SEQ ID NO: 144 - protein, 341 aa, unidentified
  REGION 1..341: note = uncultivated organism
  REGION 1..341: note = MG119-117 Effector
MINVRKLKLVIRETDKELRSQKYKFIRDSQYAQYRALNVAMGYISSAYLKANKDIKSEEYKETIKLLTNSNPIFNGIEVGTGIDTLSAVTRKARKDFSASLKNGLARGERNLTSYRRTYPLITRGRDLKFRYEDNDIVIKWVNKIEFTVITGSNNMKENTIELKHTLHKIINNEYKIKESSLMFDKNNNLILNLTFDIPDKEAHEIVPGRTLGVDLGIATPAYVCLSDNTYIRSGFGSANDFFRIRQQMKSRRRSLYKSLSLVKGGKGRKKKLQAINSLRDKERNFAKTYNHQLSYKIVKFAKDNGCEFINLEHLTKNGFEDAILSSWSYYELQNMIEYKA

SEQ ID NO: 145 - protein, 429 aa, unidentified
  REGION 1..429: note = uncultivated organism
  REGION 1..429: note = MG119-118 Effector
MTKVIKVYLISKVLNANGEEVKYEDVNKILWQLQHETRQLKNKIVQLCWEWNNFQSDYYKENGYYPKDKEVLNYSFAGYVYDKIKNLTILNSANFSLVSQDVIKAFKNSIDDIRKGNKSIINYKTKQPIDLHNKSISLSYDDKKFFVNLSLVNNGFAKENNLKRFRFECIVKDNSTKTILERCMDKIYKISGSKLSYDKKKYMWYISLCYSFENNNKINLDENKILGIDLGVKKALVASVYNDFDRLDISGSEITKFRNTIEKRRKELLRQTKFSGNGKIGHGRNTRCKSIDKISDNISCFRDCANHKYSRAVIDYAIKKGCGIIQMEELSGISEEAKPFLKNWSYFDLQNKIKYKANEVGIKVITVSPSYTSQRCSKCGNIDESNRITQASFKCTSCGYSANADYNASQNLAIRDIDKIISETRANMK

SEQ ID NO: 146 - protein, 426 aa, unidentified
  REGION 1..426: note = uncultivated organism
  REGION 1..426: note = MG119-119 Effector
MTKVVKVYLISKVLNVNGEEVKYKDVNEILQQLQNETRQLKNKIVQLCWEWNNFQSDYCEKNGSYPKDKEVLNYTLSGYIYDKLKDLTIHNTNNLSSIIQDATKAFKNSKADIMKGNKSIINYKANQPIDLHNKNISLSYDDKKFFVNLSLVNNGFAKENNLKRFRFECIVKDNSTKTILERCVDKVYKISGSKLSYDNKKKMWYLNLCYSFENNCKLELDKDRVLGVDLGIVNPIVASVYGDIDRFVIDGDEIIKFRQKTEKYRVKLLRQGKYCGDGRIGHGRKTRCKPIDRIGNRISRFRDTTNHKYSKALVEYAIKKGCGTIQFENLSGISKDKEQFLKHWSYFDLQNKIKYKANEVGINVVFVSPKYTSQRCSKCGNIDENNRITQASFKCTSCGYSANADYNASQNLAIRDIDKIISANHM

SEQ ID NO: 147 - protein, 426 aa, unidentified
  REGION 1..426: note = uncultivated organism
  REGION 1..426: note = MG119-120 Effector
MTKVIKVYLISKVLNANGEEVKYEDVNKILWQLQHETRQLKNKIVQLCWEWNNFQSDYYKENGYYPKDKEVLNYSFAGYVYDKIKNLTILNSANFSLVSQDVIKAFKNSIDDIRKGNKSIINYKTKQPIDLHNKSISLSYDDKKFFVNLSLVNNGFAKENNLKRFRFECIVKDNSTKTILERCVDKVYKISGSKLSYDNKKKMWYLNLCYSFENNCKLELDKDRVLGVDLGIVNPIVASVYGDIDRFVIDGDEIIKFRQKTEKYRVKLLRQGKYCGDGRIGHGRKTRCKPIDRIGNRISRFRDTTNHKYSKALVEYAIKKGCGTIQFENLSGISKDKEQFLKHWSYFDLQNKIKYKANEVGINVVFVSPKYTSQRCSKCGNIDENNRITQASFKCTSCGYSANADYNASQNLAIRDIDKIISANHM

SEQ ID NO: 148 - protein, 455 aa, unidentified
  REGION 1..455: note = uncultivated organism
  REGION 1..455: note = MG119-121 Effector
MITVRKIKVYCDDSTFYSFLRNEQMEQNKALNMAITLLHSANTIKSIDSGAETRILKAIEKNKQVVEKLKQDLETKKTESAKEKIIEKIKLREDLIEQDEKILEEGKVFRKGLDKEFSKIYIDTNNLYQVLSSNTKISYMRTLDLAVQKAKADYSNNFVNIVTGQQSLQNYKSDIPLMIDKKNINLFKEDNNYKIRIMLGYELNVVTGNKQNNELITTLDKIINGEYSICQSSLMFDKANRVIFNLCLDIPDKKGYEPVKDRVLGVDLGIKYPVYMSIPNTYYKKSLGSINDFLRVRQQMQDRRKKLQSELQLTKGGKGRNRKLQALDRLKEKERNFVKTYNHTLSSRIVEYAKKNKCEYINIENLTKDGFPDSILRNWSYYELQTMVTYKAKREGIIVRKVNPAYTSQKCSSCGHIDSDNRQTQEDFRCVKCGFEANADYNASINIANSTEFVR

SEQ ID NO: 149 - protein, 441 aa, unidentified
  REGION 1..441: note = uncultivated organism
  REGION 1..441: note = MG119-122 Effector
MTKVTRIPLICAVTDKDGKEIDYKDIFHLLWDLQRETRMLKNKAIQLCWEWYNFSSDYKKEHGDYPDNESILGYKGGIRSYLYDRLKGESVMYSTNISCALDSARNQFQASLKEYLRGDCSIIEYKSNQPIELYNKAILLDYHDNEYWIGLKLLSRKGEKEKSFPSCLLFKGVVNDKSTRDILNRCMDGVYKISGSKLIYDKKKKWCLNLSYSFEPEMRSLDKDKILGVNVGIYKPFMASVYGDKARLFAEGGRDSEIEVFRRKIEGRRRSILKQGKMCGDGRIGHGYKTRTAPANALEDKIARFRDTMNHKYSRAVVDYAIKNNCGIIQMEDLTGITEGEKPRYLKNWSYYDLQMKIENKAKENGIKFVKIAPQYTSQRCSKCGYIHADNRPDQPTFECLKCGYKENADYNASQNIAIKDIDKIIEASDIPKKKKKSDVA

SEQ ID NO: 150 - protein, 530 aa, unidentified
  REGION 1..530: note = uncultivated organism
  REGION 1..530: note = MG119-123 Effector
MSKKGKKDKKNKKIVLCRKLKLTILGDEQTRKEQFKLIRDDQYQQYKALNLCMDLYQANHALRKYEFKLEVDSRKELNDSIPKKEKLIEKILTLEKQIISLEEKGKLNEKDDEKLEQKKSSLNKKNDELSALEEKILKLKNNIKNSIEYQASLSEKMQEKYIDDIHTVVQNQVNLNYKDTLSLVTQKVKEDFKIALNNGLKEGKRKSTSYKRDFPLMTKGRELKFKYRDENSDDIIIKWVKGITFKVILSNKKNNDKELRHTLHKIINNEVKICDSSLQFDNKNNLILNLAMEMKVERGFTPIEGRVMGVDLGMKIPAIASFNNMNGFKKFGSIDDFLRVRKQIQARRRKLQIALTTVSGGHGRDKKIQALDRFSKSEKNFVNTYNHTISKKIIEYAKVNKCGVINLEFLSLADQEKDLFLTMSESKIKQSRFIKRNWSYFDLQTKIIDKANREGIKVLLVDPYHTSQTCSKCGHYETGQRTSQAHFKCKSCGYEANADFNASQNIAKSTQYIDKKEDSQFFKNKKSKAS

SEQ ID NO: 151 - protein, 452 aa, unidentified
  REGION 1..452: note = uncultivated organism
  REGION 1..452: note = MG91B-1 Effector
MSGEGRYILTLPLKTELYQEHILEKRFRLGEELYNKFLSYEKKKYYEMTKRRDYRDAQSLIKDLTMQINELDKQSDKSKEFKKLSLDLKKKRKEQYDAIYNIKKMFNWSEFGFGMDMKKYRGYYKKNLNSQICNNLAVRCFRAFEKMEEELIKDQYRSDEDKVNPYVHYKRKDSLRSLCGSQNTTGIRFILDDEKKVGLVKWQGLEFIIDLTKCTLYEWKALQSEICYCAVKREKIKGKWKYYIQITLKGHVPDKFNKQTGELKRQLGKGNVGLYFTSTSLTVSTEDGTKTYPLAIKNHEDKKTELLQKMDASRRATNPDNYNDDGTSKEKNEIKGWHFSKAYKKYRAELYEIFRKECEEKKLLQEVLANEVIASGDVFNCNKMDFKFLQKNLGKKIQSASPAMLKTTIERKLSYHDVSINEIGYSKLNPIFEEKNVSKKDLEEVAALIRQF

SEQ ID NO: 152 - protein, 512 aa, unidentified
  REGION 1..512: note = uncultivated organism
  REGION 1..512: note = MG91B-2 Effector
MKKSSTDTCCLTLPLKLEKWQEDRLAKRFEIARQLYNTLVHAELKRLAQVKQTAEYRENERKIRTLDWNDPGDQAKLKGLYKERRKSLKTAGFTDYSFTTDMGKYYKHFRDNIGSSVAVHGIAPQVWAAFDKLLFKKGGKKVHYKKPGEVNSVQGYSRTGKSGGREIMFRGAYIEWKGLKLPLKLSPDNAYETDMLQRRVKLVRLVRRPGKRRDRWYAQLVLEGKPAIKADPVTGKAVHPIGSGPVGLDIGPQTLAYSAAGVAGLVELADRVQNIEQEKRCLQRKMDRSRRATNPDNYAEDGTIRRGVKLTHSKSKRYRRSQQELKYLQRRQAEIRKLQHIQLANHLLSLGDHFYVEKMEWSSLTHRAKKTVISEKTGKPRSKKRFGKSIANKAPAMLIEILRQKCKSLGLPNVVEINPADIKASQYNHLTGEYVKKSLSRRWNEMQDGEHLQRDLYSAFLLQHYDSMSKALDLDSLARDYPAFVQYHHREIERLHALPKTPSSMGIRRSIA

SEQ ID NO: 153 - protein, 511 aa, unidentified
  REGION 1..511: note = uncultivated organism
  REGION 1..511: note = MG91B-3 Effector
MLVSKRSTTDTCCLTLPLRLEKWQADRLAKRFEIARQIYNTLVRAELKKLVRLERISAYKDIQQQLYSLYRDGHQNSREYKVLFQKRRSLLTEAGIREYGFKGDIAKYYKHFHENIGSSVAVHCIASQVWCAFDKYLFHNGKQVHFKRAGDIRSLKGYSVSGKSGGTEIIYRGTFIEWKGLRLPLKLDPDNRYEAEMLTKRVKYVRLLRTRGNHQDRWYAQLVLEGTPAVKHDAKTNALRHPIGHGAVGIDIGPQTIAYVAQDEAALMELANRVQNIEREKLLIQRKMDRSRRATNPDNYAVDGTIKRGVKLTRNKSKRYCTLQHRLAFIQSRQALIRKVQHTELANHLLSLGDRFYVENMEWSSLTHRASETAISEKTGRYKRKKRFGKSISNKAPATLIGILEQKCKSLGLPGVIKVPTKLCASQYNHMTDTREKKPLSQRWNDMPDGHRIQRDLYSAFLLQHVTPQEDGFDRPSLQRDYEKFVSLHDEVIRQLTLAPKTLSSMGIRRT

SEQ ID NO: 154 - protein, 512 aa, unidentified
  REGION 1..512: note = uncultivated organism
  REGION 1..512: note = MG91B-4 Effector
MPTKQKEGGNRCVLTLPLLTEPWQEHIIEKRFKIIEHLKNQLISIELRKLKKLQRTKAYKNLIKQIESVPKSERTVLYKERNKMLKDAGFSQYTFMDDMAEKKSLMQKHFESHINVQIAHKAAIDVWSSFEKFLFGTGQIVHYQKRGTLSSVASKSPKTGMFYYSDENIFKWSGGGTKPKGGIILCLRVASVKTNYEKEMVANNEIKYYRVVRKWMKTKYKYYLQITFAGKPADKHREISTGRVGIDIGTQTIAIASDKSVKLLELADKVNKNHIKKCELQRKLDRSRRYTNPDNFNPDGTVKRGTKGHKLKWNKSKHYLEVAGKIRELERKNADIRKYQHTCLANDILAMGNQIYIEPMNYKGLQKRAKETQVDEKGRYKKKKRFGKSLANKAPAMFVTILKTKAEQHGGEVYEVDIMSYRASQCDHTTGEYTKHGLNDRWRILGNGDKVQRDLYSAFLIKNSNKTHDEIDLDKCSKTYDSFKKLHDKEIENIRNEVILRKRRNISSYGIA

SEQ ID NO: 155 - protein, 534 aa, unidentified
  REGION 1..534: note = uncultivated organism
  REGION 1..534: note = MG91B-5 Effector
MGWKSKGRGGTLVKKSSTDTCCLTLPLKLEKWQSDRLEKRFEIGRQIYNTVVRAELKKLQQVERTPQYREIEQKLKEAKQEEAKELWKTRTQLLSKSGFSEYGFKSDMKYYYQHFKENIGSSVAVHGIASQVWTAFEKVLFSKDGKMVHFKKKGELSSLRGYSQAGKSGGIEIIFRGSYVEWKGLKLPLKLSPDNDYETEMLSKRVKYVRILRKEGKTKPHWYAQLVLEGKPAIKRDPASGAPKHPVGHGVVGIDIGTRTLAYSADSKVDLIELADRVQNIEQEKMLLQRKLDRSRRATNPENYKPDGTIKKGVKLTRNKSKRYRKIQHQLAMLQHRQADIRKRQHNELANYLLTLGDCFFVEDMDYRALTRKAKKTEISEKTGRYKRKKRFGKSIANKAPSMLIALLDQKCKSLNLEGVKKVDTAVKASQYNHQTDECKAKELKERWNIMPDGERIQRDLYSAFLLQHCRRDLPEYNRKKLQETFGNELYRVFDREALQRDYPKFVEYHRETIQRLSALPKTIASMGIRRIIS

SEQ ID NO: 156 - protein, 511 aa, unidentified
  REGION 1..511: note = uncultivated organism
  REGION 1..511: note = MG91B-6 Effector
MKKSNTDTCCLTLPLKLEKWQEDRLAKRFEIARQIYNTMVHAELKKLRNIENSQPYRQIQKQIEALNWKNQNDKARLKSLYNDRNKLLNTIGFSEYGFKADIKYYYKHFNDNIGSSVAVHGIAPQVWAAFEKMLFHKEGKKVHYKKKDDIHSLHGYSVTGKSGGVEIIFRKTYIEWKGLKLPLKRSPDNAYETEMLGRRVKYVRLLRKPGKRKDCWYAQLSLEGKPAIKRDPESGKRRHSIGRGAVGLDIGPQTLAYSACGAADLVELADQVQNIEQMKRRLQRKLDRSRRATNPDNYADNGTIRRGVKLTHNKSNRYRRTQRELRYLQHCQAEIRKRQHIELANHLLALGDCFYVEDMAWPSLTHRAKETAISEKTGRIKRKKRFGKSVANKAPATLIGILEQKCNSLGLPGVMKVPTSLRASQYNHQSGEYTSKPLSQRWNNMPDGERIQRDLYSAFLLQHYDPKAGNFDSALLQQDYPQFVQYHHRAIQRLSTVPKTLSSMGIQRSLR

SEQ ID NO: 157 - protein, 515 aa, unidentified
  REGION 1..515: note = uncultivated organism
  REGION 1..515: note = MG91B-7 Effector
MAEETGRCVLTLPLLTEPWQEHIMEKRFSIVEHLKNQLIAMELRKLKNLQRTKAYRELAEKIEATPKEKRAPLYRERKKMLQDAGFSEYAFKHDMQSPENPMQQHFSMHITSKVAERAASDVWRAFQKYLYSNGNEVHFHKRGTLKSVACQANGSGMYYQDDTFAWHRGKTAKVKPAKDNPQGRKKRDKNKQLHIKVRPPQNAYEREMLKKEVKEIRVVRQWVKSRYKYYLQLTLVGPPVPKERKLGVGRVGIDIGTQTIAISSETSVQLRELAPGVNDNHEKKLRLQRQMDRSKRATNPENYNPDGTIRRPPKGERMRWNFSKRYKKLAGKVRELERKNAAIRKYEHTCLANEILSLGDEIYVEKMSFSGLQRRAKETKKDAKGCYLKKKRFGKSLANKAPATLMQILKTKAALTGQVAEEVNTTAFRASQFDHTSGQYRKPLLSERWKTLSGGERVQRDMYSAFLLMNSDSDKTQSDAELCSLTFENFRRLHDAEVTRIRNEHKRHLRSFGID

SEQ ID NO: 158 - protein, 527 aa, unidentified
  REGION 1..527: note = uncultivated organism
  REGION 1..527: note = MG91B-8 Effector
MNEVFVLELPLQVEKYQEDILDKRFEQLRQLYNYVQGKLLRQYRYFEQTKEFQACKTIKEKRDFFRNHPFHFNGIYGNNNELFNITFDEYGVNGFVSKMAKKQVGANMTYADLGITTTLLDRLSLAIWASWEKKMYDYHTKRISFKKHGELNIFGSRKKTNGSGKKMFSGMELNLERMEVSIKLNGKVGKKAKYITLPILHNPKHVDYEMMALKGGFDSVKVITVVRRLVRGRYKYYLQMSIEGEKPKKDRTLGKGNVGIDIGPTTVAVSGENIASIDKLASKCDSIEAEKARLGRKIDRSRRANNPQNFNEDGTIKRGVRLVWNDSKRYKVLRREMTELQRRQAAIRKQQHIDRANELLKEGDTFIVENNQISGWTRKAKETKVNEKTGKIQKKKRFGKSVANHAPSMFVTILENKVKSLGGQVVKVDTKNAASQYDFTNDSFEKHELNERSVTLSNGDTHQRDMLAAFNLQHLKYDAQEKKQYDREAMTQHYDRFCKLERAEIMRYKNKEKRDDRSTIGAGELNT

SEQ ID NO: 159 - protein, 518 aa, unidentified
  REGION 1..518: note = uncultivated organism
  REGION 1..518: note = MG91B-9 Effector
MDKEVFVIEFPLNVEKWQADILNKRYEHLRTLYNYVQGKLLRQYIYFAQMGEYKNCKTPKERREFFKTHAFHIKGISGRNNITFTEYGIHSLVTKLAQHRIGADCTYRDKGINSMILDHMAAYIWSSWNKKLYDINARRISFKNFGEINTFGVRKINGARFCGLNLHLDIMTLDIKINGKNGQQAKFISLPVRYNLKHAGYEMNALAGGFDSIKVLTVVRRMVRGRYKYYLQMTIEGVKPQKGRHLGNGRVGIDIGPTTIAVSSNSTVSIDKLAAKCDNIEQDIQRIGRKIDRSRRAMNPDNFNADGTVKRGVRLTWVKSERYKALCQEKAELQRKQAAIRKAQHIERVNTLISEGDTFIIESNPIKGWTARAKETKVSEKTGRFQKKKRWGKSVANHAPSMFVTILENKVKSLGGTVIKADVKNAASQYDFTDGTFTKHELDERGVTLSNGDTHQRDMLSAFNLQHLIYDTKELKQYDQQQMEIHYPRFCELEQQEIEKYKQRIKTDDRSTIAASRL

SEQ ID NO: 160 - protein, 524 aa, unidentified
  REGION 1..524: note = uncultivated organism
  REGION 1..524: note = MG91B-10 Effector
MNEVFVLELPLLVERYQADILNKRYEQLRQLYNYVQGKLLRQYRYFEQMKEYKACEKIKEKRDFFRAHPFHINGVLNGNKKQLDITFDKNGINGFVEKLAQKSAGPTITYADLGFTTYILDHLSASIWAAWEKKLYDFKAKRISFKKYGELDSFSSRRKVVGGKVYFSGMELHLDTMKLSIKMNGKQGDNAKFITLKMLHNPKHADYEMWALKGGVDSVKVVKVVRRLVRSQYKYYVQLTIEGEKPQKGRKLGNGNVGIDLGPTTVAVSGENVVSIDKLASKCDNIQEEITRLSRKIDRSRRANNPQNFNEDGTIKRGIKLVWNDSKRYKELRRELAELRRKQAAIRKQQHIERANALLKEGDTFIVENNQISGWTTRAKETKVNEKTGKIQKKKRFGKSVANHAPSMFVSILENKVKSLGGEVVKVDTKNAASQYDFTDNSFTKHELKERNVTLSNGDTHQRDMLAAFNLQHLKYDEAEAKQYDTEAMAANYERFCQLEQEEIQRYKNKEKKDDRSTIGAEEL

SEQ ID NO: 161 - protein, 509 aa, unidentified
  REGION 1..509: note = uncultivated organism
  REGION 1..509: note = MG91B-11 Effector
MARCTTPTCVLTLPLRLEKWQEDRLAKRFEIARKIYNALLGAELKKLRRLEQTPEYQAIKKELAALYSEKDKNADRIRALYKERNKLLKDNSFTEYGFTVDAKFYYKHFRTNIGSCVTVHCIAPQVWSAFEKYLFGNGKRIHFKRPGEIYSLRGYSSKASSGVEIIYRGDHIEWNKLHLPLKLDPGNQYEEEMLTNRVKYVRILRRPGKNRDHWYAQLTLDGLPAIKRDNETGDPVHPIGHGPVGIDIGPQTIAYSSGKEAGLLELADRVQSIEDEKCRIQRKMDRSRRATNPGNYNLDGTIKRGVPLTRNKSKRYLRLQKELASIQHQQAAIRKEQHVALANYLLTLGDRFYIEDMNWQGLARRAKEATIPEEEKPHKRKKRFGKSIANKAPASLVTILKNKCTSLGLPGVIPINTKVRASQYNHLTDSYQKKELSQRWNEMPDGRKIQRDLYSAFLLQHMNSQLDGFDRAALQRDYESFVSLHDQAILRAAAAPKLISSMGVKRTVN

SEQ ID NO: 162 - protein, 614 aa, unidentified
  REGION 1..614: note = uncultivated organism
  REGION 1..614: note = MG91B-12 Effector
MAKENFVLTLPLKVEKCHADILDKRYELLRQIYNMVQQKLVRQYIYFSQQKAFKACENFDQKREFFNKHPFHFKGIKGKNGELSDIKFPYTYESRSSSSGKRPSGGISDYVAKLWSHKVGADKRLVDFGLNASIMEELGLHIMKAWEDRLYKFKAKRVSFKEYGEINTLGCRLKGNFAGFTINLEEGFITFNTNGRHGKFADLIKLPIDFSREKRGYAEDALKGGVDSLRKLVIVRRCIRGRNKYYLQLTISGEKPQKGRTLGIGDVGIDIGPSTIATSSLQGVHFDKLAEKCDNIEHELYLINRKLDRSRRANNPDNYNHDGTIKRGVRLVWKKDKDVKRYASLKQQRKELFRRQAEIRKLQHLEMANAMLSQGDTFIVENNPIDAWVRRAKETKKTKTGKNRSKKRYGKSVANHAPAMFVTILENKVKSLGGVFYKVDPGYAATQFDFTNGVFTKHEVSERSITLSNGRTHQRDMLSAFNLQHARLEVEKAKSRDKEKKVESYDIGQMQADYPIFCRLETAEQERFKNARRNERDALYFVGALAKELRHAPQEETVANRHNGGVGRKHCNTLCPENAKITGNGLVRESVARVSEAPEECYHTGDQPIILDGL

SEQ ID NO: 163 - protein, 508 aa, unidentified
  REGION 1..508: note = uncultivated organism
  REGION 1..508: note = MG91B-13 Effector
MGKGNKNGEDDTSYCVLTMPLYPEPWQSEILETRFQIMEHLENSLTALELRKLKNVERTKKYREVMSAIKEEKDDKNRNKLWKERKKLLKDAGISQFDFIKDISSLQKHFAPHIAQKVGAAAAHSHVWSAFDKVVFGKGQRIHFHKKGDLKSITSGYFGVSMTVKNGYFIWSGGTKDEKDRKVKPITISVKIKQPETYYEKEMLQKQIKYVTIVRKWIKNRYKYYLQFMLVGNPVVKPRVIANGKRVGIDIGPSSAAIVSEKEVKLVELADKVQKNHDKKTQLQRSMDRSRRAMNPQNFNKNGTIKRGIKLIWNESNHYKEMRGQIRELERKDADIRKYQHTCLANYVMSLGTEVYVEQMNFKGLQKRAKETKYDENGRPKRKKRFGKSIANRAPAMFITILNNKLSGIAGEELHKVNTFTFKASQYDHTNDTYKKKLLKDRWAELSNGDKIQRDLYSAFLIMNSDAEMQQADLRKCEETYKRFKEQHDGLIVKMKESNHIRLSSFGF

SEQ ID NO: 164 - protein, 508 aa, unidentified
  REGION 1..508: note = uncultivated organism
  REGION 1..508: note = MG91B-14 Effector
MADKECCVLTLPLQTEIWQEHIIEKRFAIMEHLKNQLIAKELRRLKNLERTKEYRNLIDEINNTPKEKRKALFSKRRKLLKDSGFSEYDFKDDIAGKNSLMQKHFIEHIATQIAHKLASDVWRSFDKFLFGNGRFIHFSKRGSLRSVASQKNGNGMTYKNGYFIWSGGQSKNKIILKIKVTPPKNEYEKEMLSKKIKYLRIVKKWVKTRYKYYLQFALEGKPVKKDRIVNKGNVGIDIGTQTVAISSADSVKLLELADKIDKNHTKKKILQRKMDRSRRYTNPQNYNEDGTIKKPPKGQRFSWYKSKKYIQLAGKVRELERKNADIRKYQHTCLANWILSLGDTVYVEQMNFSGLQRRAKETKIDKNGKYAKKKRYGKSLANKAPSMFLTILESKLNQYGGQLNKINTYEFKASQYDHTDDTFTKHNRSERWHILSNGDKNQRDLYSAFLIMNSDISLEHCNREKCNETYSNFKVLQDKEIERLYSDIRKGNCKNISSFGFQRNAKAM

SEQ ID NO: 165 - protein, 505 aa, unidentified
  REGION 1..505: note = uncultivated organism
  REGION 1..505: note = MG91B-15 Effector
MAKATTETCCLTLPLKLEKWQADRLDKRLEIARQIYNTLLRFELKKLRILINSEEYKDIENKRSETTDTNEKKKLNKKLDDLRKKAGFTEYSFKTDIKDFYKHFKDNIGSSVAVHGIAPQVWQAFDRLLYGNGREVHFKKRGSINSLRGYSVAGKSGGVEIMFRGDHIEWKGLHLPIKLDPNNVYECEMLQKRIKYCRIVKKQGRSKDHWYVQLMLEGKPAVKCDRHTGELLHPIGSGAVGIDIGPQTIAYVADKEVALKELADKVNNIEHKKQILQRKLDRSRRAANPDNYAPDGTIKRGVKLTHNKSKRYISTQRELADLMRKQAETRKRQHNELANHLLSLGDRFYVEDMQWPSLTHRAKKTEISEKTGRYKRKKRFGKSVGNKAPAMLISILDQKLRSRGKPGVTKVPTSVRASQYNHISDDYHKKELGERWNDMQDGRRIQRDLYSAFLLQHIDTDNNNFDKPTLFRDYENFVLLHDQYIEELKKLPKTLASMGIVRKAS

SEQ ID NO: 166 - protein, 521 aa, unidentified
  REGION 1..521: note = uncultivated organism
  REGION 1..521: note = MG91B-16 Effector
MAKEVFVIEFPLKVEKWQSDILNKRYEHLRQLYNYVQGKLLRQYIYFTQMGEYKACKSLKEKKDFFKEHIFHFKGIVGRNKKPLDISFTQYGISGLVSKLGQFSIGGKTYNDIGIGSTILANLAANVWASWDKRLYDIECERISFKKYGELNTFGSGKINKKYFCGFDLHLDRMKLDIKINGRTGKQAKFISLPIRYNLKHVDYEMNALAGGFDSIKVLTVVRRMVRGQYKYYLQMTIEGVKPQKGRHLGKGRVGIDIGPTTIAVSSNSTVSIDKLAAKCDNIEKDIQRINRKLDRSRRTMNPDNFNENGTIKRGVHLIWNNSENYKALRRQKAELQRKQAAVRKMQHIERANSLISEGDTFIIESNPIKGWTARAKETKISEKTGRIQKKKRWGKSVANHAPSMFITILENKVKSLGGTVIKADVKNAASQYDFTDGTFTKHELDERGVTLSNGDTHQRDMLSAFNLQHLIYDAKELKQYDQQRMKSHYPRFCELEQDEIERYRNKVKKDDRCTIAASRL

SEQ ID NO: 167 - protein, 566 aa, unidentified
  REGION 1..566: note = uncultivated organism
  REGION 1..566: note = MG91B-17 Effector
MGWKSKGRGGTLVDNEEKKEDGATCCLTLPLKLEKWQSDHLEKRFEIARQLYNTLVHAELKRIHRIERMPEYREIERQIEATTDKKEKWALKNQKKQFLRKNGINRNTFTTDMKRLYKHFNDNIGSNVAQHGIVSQVLTAFEKVYIENQSGEVHFKKPGDLRSVQGYSQKESGGKEIMFRNGKIIWNQQENDDRKKSPKYQQRKKRQNYKGVKGIAREFPVVFSKNNPYEAEMLTKHVKFVRILRKDGKTKPHWYAQLVLEGTPVAKYDPISGAPKHPVGHGVVGIDIGTRTLAYSTDKEVNLLELADRVQNIEQEKRRLQRKMDRSRRAMNPENYNSDGTFKRGVKLTRNKSKRYRRIQHQLAMIQHHQADIRKQQHNELANYLLTLGDCFFVENMSYCDLVHRANKTEISEKTGRYKRKKRFGKSIANKAPAMLITMLKQKCQSRGLKGVKEVDTHVRASQYNHQTDTCIEKALENRWNVMPDGERIQRDLYSAFLLQHCKQGLPEYNRKELKKEFGEELYRVFDREALQRDYPQFVAYHHMTIQRLSELPHTIPSMGIRRIIS

SEQ ID NO: 168 - protein, 512 aa, unidentified
  REGION 1..512: note = uncultivated organism
  REGION 1..512: note = MG91B-18 Effector
MAKCSTDTCCLTLPLRLEKWQEDRLAKRFEIARQIYNTMVRAELKKLRAIEQSEPYKANQEKIQALDWKTQADKQALKALYKERDKLLRNGGFSEYGFKYDIKNYYMHFNNNIGSNVAVHGIAPQVWAAFEKMLFKKEGKKVHFKKKGDIRSLRGYSVTRKSGGVEIIFRETYIEWKGLKLPLKLSPDNIYETQMLSYRVKYVRLLRKPGKRKDRWYAQLSLEGKPVIKYNPKTGEVRHPIGKGAVGLDIGPQTLAYSAATGADLVELANQVQNVEQEKRRLQRKLDRSRRATNPGNYADDGTIRHGIALTHNKSKRYLRTQQELKYLQHQQAEIRKRQHTELANHLLSLGDCFYVEKMVWTSLTHRAKETEISEKTGKIKRKKRFGKSVANKPPATLIGILQLKSAALGIPGVIEVPTSVKASQYNHQSGTYTPKPLSQRWNNMPDGERIQRDLYSAFLLQHYDPQTNTFDANSLHQDYPQFVQYHHDAVLRLYAMPKTPSSMGIKRRSLR

SEQ ID NO: 169 - protein, 548 aa, unidentified
  REGION 1..548: note = uncultivated organism
  REGION 1..548: note = MG91B-19 Effector
MEWKSTGRGGTLVDNEEKKEDGATCCLTLPLKLEKWQSDHLEKRFEIARQLYNTLVHAELKRIHRIERMPEYREIERQIEATTDKKEKWALKNQKKQFLRKNGINRNTFTTDMKRLYKHFNDNIGSNVAQHGIVSQVLTAFEKVYIENQSGEVHFKKPGDLRSVQGYSQKKSGGVEIMFREGKIIWNQQGNNDRKGVAREFPLKLSPDNSYEAEMLKKHVKFVRILRNEGKTKPHWYAQLVLEGKPAIKRDPASGAPKHPVGHGVVGIDIGTRTLAYSTDSKVDLIELADRVQNTEQEKMLLQRKLDRSRRATNPENYKPDGTIKKGVKLTRNKSKRYRKIQHQLAMLQHRQADIRKRQHNELANYLLTLGDCFFVEDMDYRALTRKAKKTEISEKTGRYKRKKRFGKSIANKAPSMLIALLDQKCKSLNLEGVKKVDTAVKASQYNHQTDECKAKKLKERWNIMPDGERIQRDLYSAFLLQHCRRDLPEYNRKKLQEAFGNELYRVFDREALQRDYPKFVEYHRETIQRLSALPKTIASMGIRRIIS

SEQ ID NO: 170 - protein, 511 aa, unidentified
  REGION 1..511: note = uncultivated organism
  REGION 1..511: note = MG91B-20 Effector
MGKSSTDTCCLTLPLKLEKWQEDRLAKRFEIARQIYNTMVRAELKRLRQVEQSPQYRSNKKAIQALDWKNTADKPKLKTLYRERDKLLKGGGFTEYGFKSDIKNFYKHFAENIGSSVAVHGIAPQVWAAFERVLFKKEGKKVHFKKTGDIRSLRGYSVTGKSGGVEIIFRGSYIEWKGLDLPLKLSPDNAYETEMLSYRVKYVRILRKPGKTRDRWYAQLSLEGKPAIKRNPASGNPVHPVGQGAVGLDIGPQTLAYAAEKEANLVELADRVQNIEREKHLLQRKMDRSRRAANPDNYAENGTIRRGVKLTHNKSKRYLRLQRELCYLQHCQAETRKRQHTELANHLLSLGSCFYVEDMAWPSLTHRAKKTEISDKTGRIKRKKRFGKSIANKAPAALINILRTKCVSLGIPGVVEVPTSLKASQYNHQSKEYTPKPLSQRWNDMPDGSRIQRDLYSAFLLQHYDPQSGNFDQASLEADYPQFVQLHNQSIQRLLTVPKTPSSMGIRRSVS

SEQ ID NO: 171 - protein, 466 aa, unidentified
  REGION 1..466: note = uncultivated organism
  REGION 1..466: note = MG91B-21 Effector
MAVMSVVTIPLKTRIYEEDIINKRMELCRKVYNNMLHDKLKDLRKMEHDKEYIASKSIILSAYKTDDKNEVKKIKNSDEYKKAQEVQKEKYKEYGFSEYAFASDVLKYIKLFNSNITATVATYTISKPMWAAFEKYLFADGRMIHYKSFNSISSVASDGKAGLKIVVNNKKSTRKPESPFGLSLKYGSNRAGKTINLPLIVDKNDFYMQEMICNNIKVVRLVRRKIKTHYKYYVQLTVVTAPSIKFDRCTGEIKNPINDGNVGIFIDTRKITLCHKNGLKQIDLIDKRQIKFEEEIGNINRFLDNSRRISNPENYNEDGTIKKGIVVNGQKRKLHWNNSNNYKKALAKKADIQRRLAEQRKIRAGEITNMILSLGNNIICNNYNFKAAQMRKKETEIDEKGKIKTKKKAGKAISYSAPAMLLTILNNKLVARGYNEIYKVEVDKDMTITDQTRILKAQELYMQLEN

SEQ ID NO: 172 - protein, 519 aa, unidentified
  REGION 1..519: note = uncultivated organism
  REGION 1..519: note = MG91B-22 Effector
MPTKQKEGGNRCVLTLPLLTEPWQEHIIEKRFKIIEHLKNQLISIELRKLKNLQRTKAYKNLIKQIESVPKSERTALYKERNKMLEGKGFSRNVFQDDLGVMQNQFAEHINVQISHRAAWDVWSSFEKFLFGSGQMIHYQKRGTLKSIASKSPKNGMHYFPESNTFKWSGGRQKKIGQKGEQKKVSIKPISLCLRVSPLKTDYEKEMVANNEIKYYRVVRKWMKTKYKYYLQITFAGKPADKHREISTGRVGIDIGTQTIAIASDKSVKLLELADKVNKNHIKKCELQRKLDRSRRYTNPDNFNPDGTVKRGTKGHKLKWNKSKHYLEIAGKIRELERKNADIRKYQHTCLANDILAMGNQIYIEPMNYKGLQKRAKETQVDEKGRYKKKKRFGKSLANKAPAMFVTILKTKAEQHGGEVYEVDIMLYRASQCDHTTGEYTKHGLNDRWRILGNGDKVQRDLYSAFLIKNSNKTHDEIDLDKCSKTYDSFKKLHDKEIENIRNEVILRKRRNISSYGIA

SEQ ID NO: 173 - protein, 536 aa, unidentified
  REGION 1..536: note = uncultivated organism
  REGION 1..536: note = MG91B-23 Effector
MDADEYKKLLKKFHKLSDRHILVAEADMSYPDVQKVVNLSDRIRKAGNELVSLMKKNYEQLMRTKKYRKLLQLYGSTEDKKKRKTYAGQLNEMQEQYNVTWDFCRASMIPIGKKYGIDAIFALTKAEDVWRGVEKCLYGNGRMLHFSKYGDFPCIRAKQINRGIPMSVKNDELKFKLGKLVFGIQVKDRFQTDEVNAVLDYLAQPEVIDNKAVKTLQDEACCISTYRPCYATLVPKFIRGKYRVYLHLVIEGKAKPKYDSSGNPRHKYGRGVVGADIGTQTVAYTSDTETGLKNLSERGNSIQKSERLERLYYRAMDRSRRATNPKNYNADGTIKKGKKKWTYSRHYKKLKAKHAELCCINATNRQLAINEDVNHLRCLGDTFVTEPKNAARLMKRAKETTVNNKGKINRKKRFGKSVKNRCPGGFQAAVENKFKTSGGTYIEVPNNYRASQYDHTADDYIKKKLSDRMYKLADGTLVQRDWYSSFLLYCYNYRTNDIDKKKCAAEFKKCYSMEKDLIEWIKENKIKVLNSGIKIA

SEQ ID NO: 174 - protein, 849 aa, unidentified
  REGION 1..849: note = uncultivated organism
  REGION 1..849: note = MG91B-24 Effector
MKEEDIIKFSDSLKGLGTNKKGVALILRNPIKITEDSAVQGVIIDNKPDRKEGIDRLDYIVYDRENGMHRSPATEGNFDQEVPYHILSRFEDLMDCGKLIPVVFEDSVNPFSTDITFKHNDPLKNDKKAFWNYPLDKEQFEASSSVRFYKSVLAPRNFSESNTFNCKTEEASLYFAVENARRLSKRGSAVRDFRNKDIWTVSLDNADESKPLDVETAEFFATKSIRSSILNMGDTKVFQALFDSKKNAIAFASDYANWMTKAEIAVGKLLGFNIPSNIQDQGMDLLGGDKKYIDCYLQKNFYTEEFQERPFGELLDNTLKWIREYGANPDEENIKAVSESLDKIAFDKYVVEGIHLDYFFQEPITLDDGRKIVGFGKNLGKDNVTWAAKAYTTNADNTAVEKVDFKDLPHDGLKKAIEKLDSPYHPHIDFGDKKLNVYKMDRVAYSLYQRISNVGAKSFTPSEASQAELFFKQLPEEIRQPMAVYLENLVRKNINEKEVSLPEKWFTSAFEELEKIAKDGLPIKYNLKHADYEMTALAGGIDSIKLLTVVRRLVRGKYKYYLQITIKGKKPQKGRELGSGKVGIDLGASTIAVCGDNIVSIDKLAAKCDYIQKDVKKIQRKMDRSRRANNPDNYNENGAIKKGKKLVWKNSNRYKALCKQYAEIKRKQAAVRKIEHIERANNLLKEGNIFITEKNPIKEWAKKSEETEINPKTGKTKSKKKYGKSISVHAPGMFIEILKNKVVSLGGKFIKDNPENAASEYDFTNDTFTDHGFDRTITLSNGDKHQRDTLAAFNIKNRKYDEEEPKQYNREQMQKEYARFCELEREEIEKYKNGTKKDYRRTIAAYKLY

SEQ ID NO: 175 - protein, 486 aa, unidentified
  REGION 1..486: note = uncultivated organism
  REGION 1..486: note = MG91B-25 Effector
MSNFVVEFPLITEPYQEHILNKRFEIGRIIYNTLVNQTKKIYKEMIKTKKYRELQNKLSDDEKSNKLVWKEINNIRKENGFSEYSFHKLVSKSQKYFKKNIDSFTAQKIATRLWLTYDKFFYGNGEKIYFKKFGTLNSLEGKSNSSGIRFKNNKIYWNKLIIPVKINYSNSYEYEALQHKICYNRIIRKYVKNKYKYYVQIVFEGIPPIKINQNTGEVKHHIGNGDVGLDIGTSTIAISSMTDVKIFELADKVKNIEREKLLLQRKMSRSRFSTNPNNYNSDGTIKSQGNRKVKWIKSNHYIKYQKQLRELYRKQTAVRKYQHECLANYIISLGNKFFVEKMNFKALQKCTTKTEKNEKGKFKRKKRFGKSLGNRAPSMLLTIINRKLSYYGTELIEINTYKAKASQFNHIDNTFKKKKLSQRWNDLNGIKVQRDMYSAYLIMNINKDLETFNMKKCNDRFDNFLKLHNKEVNRLKNHKNLSCIAI

SEQ ID NO: 176 - protein, 495 aa, unidentified
  REGION 1..495: note = uncultivated organism
  REGION 1..495: note = MG91B-26 Effector
MSDSSTEKSVFVVTFPLKTEKWQEDRINKMMRLLTVFYNEKQKVLLERWMHIRNSAEYKEHKEEKSKNSLTAYMRNFGFSEFGFKEFFKDKNQTDSPYITHGLNSAILRNLAKSAWSAWSKKLEEKKKNIFIHTDKDVSIIKSDFKSSNNKISGFDVNYEDYSITMSASKPHVHTMFTIPFVVDRNSEYELFALGEIQNDPTKLRNLAIVRKEVRGKYKYYVQFSIAGKPYNKGRQLGSGVVGIDPGPSKIAVVSDTAVRIIPLAKSIERDERETRRLQRKLDRSRRAMNPDNYNEDGTLSKGKHEWVKSHHYEETRSILADRQRKLAAKRKIAHNELANELLQMGNEFHVENNSFRSMQVRAKETTKNANGKNRTKKRFGKSLANRAPSEFLIILENKVKQYENGVYVDIPDSIACTQYDFTSGDFSAHELRERTITTSDGIRHDRDALAAFNMKFVRTEQVVEKKKVKKAVDNFDNESMAEFYPLFCQMENKE

SEQ ID NO: 177 - protein, 510 aa, unidentified
  REGION 1..510: note = uncultivated organism
  REGION 1..510: note = MG91B-27 Effector
MFVVELPLKVEKWQSDVLNKRYELIAKIYNYAQKKLLRQYIYLTQMDEYKKCKTKKEKSNFFKNHPFYFKDITSKITFSEYDVTAFVEKIIKKNVSSDKTYKDFGINTSITECLGKHIWSAWEKKIYDKGKKISFKNKGELNTISIRKKSEYFGGLDVDIEHMILSFNINGKIKAKAKKIILPINVKNLTEYEMYALQGGYDSINVVTIIRKTIRGKYKYYVQFTIEGEKPQKGRTLGKGNVGIDIGPSTVAISSLNGVTIDTLAEKCDNIESKISDIGRKMDRSRRATNPQNFTEDGQIKRIDRKKGEKRVWKCSKRYVKLRNKKHELLRKQAVIRKLQHNIKANEILSLGDTFIVEDNPTSAWAKRAKGVRKNKNGKIQTNKRFGKSIANHAPSMFVTILQNKVDSLKGKFVKVDCKNAASRFDFTNGDFTGHNLDERKITLSNGNTHQRDMLAAFNLQHIDLTNDDLKKYNVDEMVEDYPIFCKLEKEEIGKFINGQKKSRSCTVGY

SEQ ID NO: 178 - protein, 528 aa, unidentified
  REGION 1..528: note = uncultivated organism
  REGION 1..528: note = MG91B-28 Effector
MNEVFVLELPLQVEKYQADILNKRYEQLRQLYNYVQGKLLRQYRYFEQMKEYQACKTFKEKRDFFRSHPFHIDGILGSNKELFDITFDEYGINGFVGKLGKKQVSAGVTYADLGITSTLLDRLSLAVWAAWEKMLYDYHARRVSFKKYGELNIFGSRKKTNGSGKKMFSGMELHLEKMEVAIKLNGKVGKAAKFITLPILHNPKHVDYEMWALKGGFDSVTVLTIVRRLVRSQYKYYVQLTIEGEKPQKGRTLGKGNVGIDLGPTTVAVCGENVVSIDKLASKCDNIQEKITRLSRKIDRSRRANNPQNFNEDGTIKRGIKLVWKKDKDLKRYSKLKRELAELRRKQAAIRKQQHINRANALLKEGDTFIVENNQISSWTTRAKETKVNEKTGRIQKKKRFGKSVANHAPSMFINILENKVKSLGGKVVKVDTKNAASQYDFTDGSFTKHELKERNVTLSNGDMHQRDILAAFNLQHLKYDEAEAKQYDTEAMSADYERFCQLEQEEIQRYKNKEKKDDRGTIGAEKL

SEQ ID NO: 179 - protein, 525 aa, unidentified
  REGION 1..525: note = uncultivated organism
  REGION 1..525: note = MG91B-29 Effector
MNEVFVLELPLQVEKFQVDILNKRYEQLRQLYNYVQGKLLRQYRYFEQMKEYQACKTFKEKKDFFRSHPFQINETLKGNETAFKVSFDEYSINSYIEKLVQKSVGSNMTYSDLGFTTNILDHLSASIWAAWNKKLYDFKAKRISFKKYGELDSFSSRRKVVRGVVYFSGIELHLDTMELAIKMNGKVGKEAKFITLKMLHNPKHAEYEMWALKGGVDSVKVVKVVRRLVRSQYKYYVQLTIEGEKPQKGRTLGRGNVGIDLGPTTVAVSGENVVSIDKLASKCDNIQEEITRLSRKIDRSRRANNPQNFNEDGTIKRGIKLVWNDSNRYKELRRELAELRRKQAAIRKQQHIDRANSLLKEGDTFIVENNQISSWTTRAKETKVNEKAGKIQKKKRFGKSVANHAPSMFVSILENKVKSLGGEVVKVDTKNAASQYDFTDGSFTKHELKERNVTLSNGDTHQRDMLAAFNLQHLKYEEKDKTKQYDTDAMKKDYERFCQLEQEEIQRYKNKEKKDDSSTIGAGEL

SEQ ID NO: 180 - protein, 464 aa, unidentified
  REGION 1..464: note = uncultivated organism
  REGION 1..464: note = MG91B-30 Effector
MAENKIMSVVTLPLKTEKWQEDILQKRFELCRKIYNNMLHYEMKQLNKVLHDKRYIDSKKIITETYKIADDKEKRARKRTEEYKQAVKISNEVLKEYGLTDYGFKNEVLKQRAVYKTNISSTVAQLSVASPLWAAMDKYLFGNGKTLHYKKYNTLNSMASDGKSGIRMVDKDGKTIFSRESAMPIWINMSVPKGKTLLMPVIIDKKDLYKIEMMSRKLKVIRITRKLCKAKYKYYVQFTVEGRPAIRYDKDGNIKNPVGNGKVGIYIDTTSITLALENGDIKTISLQHDNTSTEKIADLQKYMDNSRRATNPDNYNADGTVKKGITIDGKKSKLRWNYSNGYNRARLELKNLYRVDAENKKLERQILANKILSYGDNFAINDYPFQYAAMRKKMFNENTLKDEKHKKAGDKIGNNAPATLVVLLDSKLKAKVGAGVTKVQLKDIDYSMENYRERYAQKILEYLQ

SEQ ID NO: 181 - protein, 456 aa, unidentified
  REGION 1..456: note = uncultivated organism
  REGION 1..456: note = MG91B-31 Effector
MSNAENTSGRYILTLPLKTELYQEHILEKRFRLGEQLYNKFLNFEKKKYFEMTKRKDYRNAQSLIKNFTYQIKSLNNASDISKDTKKMIQDFKKQRKEQYDIIYNIKKMFNWSEFGFGKDMKKYRGYYKKNLNSQICNNLAVRCFRAFEKMEEEFIKDQYRSDEDKVNPCVHYKRKDSLKSLCGSQNTTGIRFILGDERNVGTVKWQGLEFSIDLVKCTLYEWKTLQSEICYCAVKREKIKGKWKYYIQLTLKGHVPDKFNKQTGELKRQLGKGNVGLYFTSTSLTVSTEDGTKTYPLAIKNHEDKKTELLQKMDASRRATNPDNYNDDGTSKEKNEIKGWHFSKAYKKYRAELYEIFRKECEEKKLLQEVLANEVIASGDVFNCNKMDFKFLQRNLGKKILTASPSMLKSVIERKLSYHDVSINEISYSKLNPVLEEKNISKKNLEDMATLISQF

SEQ ID NO: 182 - protein, 485 aa, unidentified
  REGION 1..485: note = uncultivated organism
  REGION 1..485: note = MG91B-32 Effector
MKRFEIARQIYNSLLHAEQKKLRRLEQTPEYREIQRKLQELYREKAEKSRAFREIAKKRTALLKRAGFTEYAFKTDMKAYYKHFKENIGSSVAVHGIASQVWTAFDRKLYGNGKHVHFKRPGEIRSLRGYSAAGKSGGVEILFRGTHIEWKGLCLPLKQKPENAYETEMLSRRVKYVRILRTPGKHRDRWYAQLVLEGRPAVKRNTETGELLHPIGKGPVGIDIGPQTVAYVSQGEASLLELADRVRNIEQEKRKLQRKMDRSRRATNPENYAEDGTIRRGIRLTRNKSGRYRKLQKELAFLQHHQADIRKQQHIALANHLLSLGDRFYVEDMAWPSLTHRAKETVISPKTGRYQRKKRFGKSVANKAPAMFLGILTQKCVSLGLPGVVKVSTSVRASQYNHLTETYQKKPLSQRWNVMPDGRKIQRDLYSAFLLQHMTPQLDGFDPDGLRQDYESFAVLHEEAIHKLRLVPRTLTSMGLQRKCS

SEQ ID NO: 183 - protein, 537 aa, unidentified
  REGION 1..537: note = uncultivated organism
  REGION 1..537: note = MG91B-33 Effector
MIIDGGYKKLLKQFHKHSDRHILVIETDMPYSDILKVVALSDKIRKAGNELVGLMKKNYAQLLRTKRYRKLLFLYGNTEDKDKRKIYANQLNEMQKIYNVTWNYCRTSMIPIGKKYGIDAVFALTKAEDVWRGMEKCLYGNGNTVHFSKHGDLPCIRAKQISRGIPISVRNDKLQFKLGKTSFGIQVNDRFQQDEVNAVLSYLVEPEIMDNKAVNTLIEDAYCIDTYRPCYATLVPKMIRGKYRVYLHLTIEGKTKLKYDKHGNPRYKYGKGIIGADIGTQTVAYTSDTEVGLKNLSERGNSIQTSERKERLLYRAMDRSRRATNPQNFNEDGTVKKGRKTWKYSNHYKKLKTKHSELCRINAVNRQLAINEDANHLRGLGDTLVTEPKNASKLMKRAKETTVNSKGKFNKKKRFGKSIKNRCPSGFQTTVEKKFKTTGGTYIEVPNNYRASQYDHTVDDYIKKKLSDRLYKLQDGTEVQRDWYSSFLLYCYDYKIQNIDKNKCITEFDNCYKKEKALITWIKTHKIKVLNSGIKTA

SEQ ID NO: 184 - protein, 499 aa, unidentified
  REGION 1..499: note = uncultivated organism
  REGION 1..499: note = MG91B-34 Effector
MADNDARCVLTLPLITEPWQEHIIETRFRIMEHLKNQLIAMELRKLRNLRRTREYRALEEDIANTPKGSRGELRRKRRKILRDAGFSEYDFKDDMAGTNSKMQKHFAEHIAAQIAHKAASDVWRAFDKCLFGSGKGIRFHAHNTLSSVACQEIGNGMSYVNGKFVWKGGRCENSICLSVPVKPPDTVYECEMLKKQVKSLRIVRKWMKSKYKYYLQFTLTGEPIKKTREVGFGNVGIDIGVQTVAIVSSSSVFLLELADKINANHSRKKQLQQKMDRSKRYSNPSNYDKNGVIIRSPSGQRLRWEFSKKYRKYAGIVRNLERKNADIRKYQHTCLANAVLPLGNRVFVETMNFSAHQNRAKETKKDSNGRFLKKKRFGKSLANKAPAMFISILKYKLALYGGNLFEVDRYTFKASQYDHTTGECTKHSLSERWRTLGNGDIVQRDLYSAFLLMNSNTSLSSCDATACAHTYTDFKTLSDYEIHRIKSEGIRLISSFGIA

SEQ ID NO: 185 - protein, 512 aa, unidentified
  REGION 1..512: note = uncultivated organism
  REGION 1..512: note = MG91B-35 Effector
MEKTKGSDDTCCLTLPLKLEKWQEDRLARRLELARQIYNTLMHAELKKLRNVQQSPEYRRMQAEMRALDWKKEADKPPLRKLYKERDKLLKKAGFTKFGFKNDMKYYYKHFNQNIGSSVAVHGIASQVWTAFDKMLFQKDGKKVHFKKYGDLHSVQGYSAAKSGGVEIMFRGTCIEWKGLELPLKRKPDNAYETEMLQKHIKFVRILRKPGKNRDRWYAQLTLAGKPAVKRDAVSGRQIHPLGHGPVGLDIGPQTLAYAAETEADLVELADLVQDIELEKRRIQRKMDRSRRAANPDNYAEDGTIKRGVKLTHNKSRRYLRLQKKLSHLQHCQAETRKRQHNELANHLLTLGDCFYIEKMNWTSLTHRAKKTEISEKTGKIKRKKRFGKSIANKAPGMLIEILKRKCLSRGLPKVAELPTTLKASQYNHLTKEYTKKSLSQRWNTMPDGKRIQRDLYSAFLLQHYNPQLQNFDQEALEKEYPQFVLLHDQAIERLSAIPKMPSSMGIRRSVS

SEQ ID NO: 186 - protein, 490 aa, unidentified
  REGION 1..490: note = uncultivated organism
  REGION 1..490: note = MG91B-36 Effector
MSSQGERCVLTLPLLTEPFQDVIIEKRFKIMEHLQNSLIAYELRKLKSLERTRAYRELCERIRNASDKERKTLSRQRSKMLKEAGFSEYQFQDDITPMQKHFVEHIFVHEAHRAAQDVWRAFEKYLFGSGKMVHFKRRGTLSSIANKTSGMYLRDGWFEWNGGQSKQSIRLKIRVMPPRNAYETEMLKRPVRNFRVVRKWMKCRYKYYLQITLEGNPVVKDRPVAEGRVGIDIGTQSVAIASLNGVCLLELADQVNKNHARIKYLQQKMDRSRRATNAGNFAPDGTIRRGIRLTWSESNHYKRLAGTVRRIQRKNADIRKYQHYCLVRDVLMLGNQVYVEKMNFQGLQRRARETTVNERGRFKRKKRFGKSLANKAPAMFISLLEQKLHSSGGNLYRVNTWEFKASQYDHLSRKYNKKKLSQRTHRLGNGDVLQRDLYSAFLLMNADSSLRFPDQKKCDETYPIFKSLHDKEVKRISNDSKYHLTSFGIA

SEQ ID NO: 187 - protein, 512 aa, unidentified
  REGION 1..512: note = uncultivated organism
  REGION 1..512: note = MG91B-37 Effector
MGKSSTDTCCLSLPLKLEKWQEDRLAKRFEIARQIYNTLVHAELKRLSQVKRSQQYREIENKIRALDRNDQEDKKELSLLHKLRDDLLRKSGFNDYGFKADIKYFYKHFGENIGANVAVHGIAPQVWSAFDSVLFKKRGKKVHYKKPGDICSLRGYSVTGKSGGSEIMFRGTYIEWKGLKMPLKLSPDNIYESEMLDHRVKYVRIVRKPGKRKDRWYAQLSLECKPVIKCDPVSGEMRHPVGKGPVGLDIGPQTLAYCALSEAGLVELADQVQDIEREKQRLQRRLDRSRRAANPNNYAADGTIKRGVKLTHNKSKRYCILQRELRYLQQQQAETRKRQHTELANYLLSLGDRFYVEDMNWSSLARRAKKTEISEKTGRYKRKKRFGKSIANKAPGSLIDILKQKCESLGIPGVEKLPTSLLKASQYNHQTGEYTPKPRSQRWNDMPDGERIQRDLYSAFLLQHYDPVSKGYDQAALQRDYPQFLQHHHHSIQRLTAKPKTPSSMGIHQRLR

SEQ ID NO: 188 - protein, 536 aa, unidentified
  REGION 1..536: note = uncultivated organism
  REGION 1..536: note = MG91B-38 Effector
MVNDDYNKVLKQFHKLSNRHILVAETDMSCSDIQKVVKLSDRIRKAGNELIGLMKKNYDQLMRTKRYRGLLKLYGSTKNKQKRKMFADQLNEMQKQYNVTWDYCRKSMIHIGKKYSIDAVFALTKAEDVWHGVEKCLYGNGKTLHFSKHGDLPCIRAKQINRGIPISAKNNQLQFKLGKSVFGIQIKDRFQTDEVNAVLDYLAAPEIADCKAVQTLLEEACCISTYRPCYATLVPKLIRGKYRVYLHLAIEGRAKPKYDRFGNPRHKYGKGIIGADIGTQTVAYTSDTETGLKNLSDRGNSIQTSERLERLYYRAMDRSRRATNPQNYNDDGTVKQGKKTWNYSNHYKKLKAKHAELCRINVANRRLAINEDANHLRSIGDVFITEPKNAAKLMKRASKTTVNNKGKINKKKRFGKSIKNRCPGGFQTAVENKFKASGGTYIEVSNNYRASQYDHTADDYIKKKLSDRMYKLADGTLVQRDWYSSFLLYCYDFRTSDIDKDKCAAKFDKCYSMERELIEWIKTNKIKVLNSGIKIA

SEQ ID NO: 189 - protein, 536 aa, unidentified
  REGION 1..536: note = uncultivated organism
  REGION 1..536: note = MG91B-39 Effector
MDNEEKEKDGATCCLTLPLKLEKWQSDHLEKRFEIARQLYNTLVHAELKRIHRIERMPEYREIERLIEATTDKKEKWALKNQKKQFLRKNGINRNTFTTDMKRLYKHFNDNIGSNVAQHGIVSQVLTAFEKVYIENQSGEVHFKKPGDLRSVQGYSQKKSGGVEIMFREGKIIWNQQGNNDRKGVAREFPLKLSPDNSYEAEMLKKHVKFVRILRKDGKTKPHWYAQLVLEGTPVAKYDPISGAPKHPVGHGVVGIDIGTRTLAYSTDKEVNLLELADRVQNIEQEKRRLQRKMDRSRRAMNPENYNSDGTFKRGVKLTRNKSKRYRRIQHQLAMIQHHQADIRKQQHNELANYLLTLGDCFFVENMSYCDLVHRANKTEISEKTGRYKRKKRFGKSIANKAPAMLITMLKQKCQSRGLKGVKEVDTHVRASQYNHQTDTCIEKALENRWNVMPDGERIQRDLYSAFLLQHCKQGLPEYNRKELKKEFGEELYRVFDREALQRDYPQFVAYHHMTIQRLSELPHTIPSMGIRRIIS

SEQ ID NO: 190 - protein, 615 aa, unidentified
  REGION 1..615: note = uncultivated organism
  REGION 1..615: note = MG91B-40 Effector
MAKEVFVLTLPLKVEKWQADILDKRYELLRQTYNMVQQRLLRQYIYFSQQKAYKACETFVQRRDFFASHPFHFKGIKGKNGESSDIKFPYTYESRSSNTGKRPSGGISDYVSKLWSHRVGQDKTLADFGINSYLLEELGLHIMKAWEKRLYDPQSSHVSFKKYGDVNSFGSRYKKDKKSNRITFPGFAIDLVKGYIVYNTEGKRVKTNPFVLPIDFSREKKEYALESLKGGVDSIRKLLIVRRCIRGRNKYYLQLSIKGEKPQKGRTLGKGNVGIDIGPSTIAVSSLQGVHFDKLANKCDNIEHELHLLNRKIDRSRRANNPQNYNEDGTVKRGVRLVWKKDKDAKRYANLKQIRKELHRRQAAIRKQQHLEMANALLSQGDTFIVENNPVDAWVRRAKETTKTKTGKNRCKKRFGKSVANHAPATFVSILKNKVLSLGGQFHEVDIHNAATQFDFTSQQFTKHELGERSVKLSNGNTHQRDMLSAFNLQHLRAQSEENKDYDIEQMKADYPIFCKLETAEKERIKGERSVERDVLFYIGASTQDMRQQPQKSTVVNRHNGGVGRKHCNTRLGSRKKENAEITGSGLVRESIERVSQVPDEYHHFGIQSVILDGL

SEQ ID NO: 191 - protein, 528 aa, unidentified
  REGION 1..528: note = uncultivated organism
  REGION 1..528: note = MG91B-41 Effector
MEEVFVLELPLQVEKYQADILNKRYEQLRQLYNYVQGKLLRQYRYFEQMKEFQVCKTFKEKRDFFRSHPFHINGIYGNDKELLDITFDEYGINGFVGKMGKKLVNANTTYADLGITSTLLDRLSLAVWAAWDKMLYDYRARRVSFKKHGELNIFGSRKKTNGSGKKMFSGMELHLEKMEVSIKLNGKVGKKAKFITLPILHNPKHADYEMWALKGGFDSVKVITVVRRLVRGQYKYYLQLSIEGEKPQKGRTLGKGNVGIDIGPTTVAVSGENIASIDKLASKCDNIQKEITRLARKIDRSRRANNPQNFNEDGTIKRGVRLVWKKDKDCKRYAALKREMAELQRRQAAIRKQQHIDRANALLKEGDAFIVENNQISGWVRKAKETKVNEKTGKIQKKKRFGKSVANHAPAMFVTILENKVKSLGGEVVKVDTQNAASQYDFTNNSFEKHELKERSVTLSNGDTHQRDMLAAFNLQHLKYDDAEKKQYDTEAMAQHYDRFCELEQAEIQRYKNKEKKDDRSTIGAGDL

SEQ ID NO: 192 - protein, 526 aa, unidentified
  REGION 1..526: note = uncultivated organism
  REGION 1..526: note = MG91B-42 Effector
MKEVFVLEMPLLVEKYQEDILNKRFEQLRQLYNYVQGKLLRQYKYFEQMAEFQICKTSKAKREFIKTHPFHINGVIGRNKDLLDISFDEYGILGLVTKLGQKPLGSGKSYADMGISSTILANLSNNIWSAWNKKLYDYHAKRISFKRYGELNSFGSGKIVQKKDNDVKEYFPGFILHLERMELDIKTSKSGKNETCITLPILHNPKHADYEMWALKDGSTSIKVLTIVRRLVRGRYKYYLQMSIEGEKPQKGRTLGKGNVGIDIGPTTVAVSGENIASIDKLASKCDNIQEEITRLARKIDRSRRANNPQNFNEDGTIKRGVRLVWNDSKRYKALRKEMAELQRKQAAIRKQQHIDRANALLKEGDTFVVENNPIKGWQARAKETKIDEKTGRFKSKKGRGKSIGNHAPAMFVTILENKVKSLGGEVVKVDTHNAASQYDFTNESFTDHGYDRKITLSNGDTHQRDMLAAFNLQHLKYDETEKKQYDTEAMVQHYDRFCELEQEEILRYKNKEKKDDRSTIGAGDL

SEQ ID NO: 193 - protein, 517 aa, unidentified
  REGION 1..517: note = uncultivated organism
  REGION 1..517: note = MG91B-43 Effector
MKKKSTEETFIMELPLKVEKFQADELNKRFELMRRIYNSAQNKLERQHEYLLSFKEYKECNTKKAKGEFINNHPFSFKGVLNKNGEPMNITFTEFGLKSYLADFAKKKVDSQYTYSDFGINTSMLMSMGMAAWSAWKKVIYDPKAKDIHYKKYGDLKSIAVDWEPKHQAGMYLDVCKGVLKLNINGRRGQYAKYLTFPIKFGRKERQLWYEGDALKGGEDSFKGLAIVRRKYNGQFRYFLQLTIRNCTPTKDRTLGKGRVAIDLGIYKIAVYTGDQVYFFPLAPNCREIDEKIAVLQRQMERSKRISNPQFYNADGTYIKAKKHEHRKWNFTKEYLKLRAMKAELQRKQAAIRKQDHIDLANHLLSFGNEFVIEDNDVKAWAERKKEEKKREKDGKNLSKKGLGKPIGSHAPAAFVTLLANKVKALRGTFTKVAVDNGATDYDFINDTFTPHELKERYITLCNGVTHQRDSLAAFNLYYIDTNPEKSRCYDRKAMKRGYKKFQELEKEALKQWKKPV

SEQ ID NO: 194 - protein, 511 aa, unidentified
  REGION 1..511: note = uncultivated organism
  REGION 1..511: note = MG91B-44 Effector
MAARSKTDTCCLTLPLRLEKWQADRLAKRFHIAEQIYNAMLLSWIKKLRVVEQMPEYRAIQSQLQKLYEEDKQKTDAAKELFSRRKEILKKNGFPEYSFKTGIRKYYAHFKENIASNVATHCIASQVWDAFSKKLFGNGGRLHFKRAGDIHSLRGYSKSGESGGVEIIFRGTYIEWNGLRLPLKLDPQNPYEAEMLAKRVKYVRILRTPGKSIDHWYAQLALEGKPTVKRNAATGEPVHPSGRGAVGIDIGPRTIAYVSENEVGLRELADQVENIERDKRRLQRKLDRSRRATNPDNYAPDGTIKRGIKHTHNKSSRYRRMQKELSLLQHRQALIRKRQHTELANHLLSLGDCFYVEDMPWKGLARRTKETTVSPTTGRYRRKKRFGKSISNKAPGTLILILEQKCKSRGVSGVIRVPTNIKASQYNHMTDTYKPKPLSQRWNDMPDGKRIQRDLYSAFLLQHVTPELDGFERTKLIEDYDGFIHLHDKEIHELTLAPKTIASMGISRTRS

SEQ ID NO: 195 - protein, 505 aa, unidentified
  REGION 1..505: note = uncultivated organism
  REGION 1..505: note = MG91B-45 Effector
MARAQTDTCCLTLPLKLEKWQSDRLDKRLEIARQIYNTLLRFELKKLLRLEESKEYTDIQNSLKEITDNTERNKLYKQIDKLRQKAGFSEYAFKTDIKEFYKHFSDNIGSSVAVHGIAAQVWTAFDRLFYGNGKAVYYKRRGELNSVRGYSVAGKSGGIEIIFRGNYIEWKGLKLPIKLDLTNTYENEMLQRRVKYCRLLKKPGRTKERWYVQLMLEGKPAIKVDKNTGELRHPVGRGAVGIDIGPQTIAYVSDSEIDLKELADQVNNIEREKRVLQRKLDRSRRATNPDNYAPDGTIKRGVKLSHNKSKRYIKTQRELAALQRQQADIRKRQHNELANHLLSLGDCFYIEKMEWPALTHRAKQTEISEKTGKDKRKKRFGKSVGNKAPATLTGLLDQKLKSRGLSGIIEVDTKVRASQYNHLTDDYHKKELSERWNFMPDGRRIQRDLYSAFLLQHMNEQQDGFDKTALQRDYERFVIQHDQCIEELKRAPKTLASMGIVRRAS

SEQ ID NO: 196 - protein, 528 aa, unidentified
  REGION 1..528: note = uncultivated organism
  REGION 1..528: note = MG91B-46 Effector
MEEVFVLELPLQVEKYQADILNKRYEQLRQLYNYVQGKLLRQYRYFEQMTEFQVCKTFKEKRDFFRSHPFHINGILGSNKELLDITFDDYGINGFVTKLGLKSIGNGKTYTDIGINSAILEKLSDNIWASWDKMLYDYKAKRISFKRYGELNSFGCRKKLNGSRKKVFSGMELRLDKMELAIKLNSKTGKNAKFITLPILHNPKHADYEMWALKGGYDSVKVITVVRRMVRGQYKYYLQMSIEGEKPQKGRTLGKGTVGIDIGPTTIAVSGENIVSIDKLAAKCDNIEAEIQRLARKIDRSRRANNPDNFNEDGTVKRGVRLVWKKDKDCKRYAALKREMAELHRRQAAIRKQQHIDRANELLKEGDTFIVENNQISSWVRRAKETKVSEKTGRIQKKKRFGKSVANHAPSMFVTILENKVKSLGGEVVKVDTQNAASQYDFTNNSFEKHELNERSVTLSDGKKHQRDMLAAFNLQHLKYGESEKKRYDTEAMNQHYERFCELEHEEIMRYKNKEKKDDRSTIGAGDL

SEQ ID NO: 197 - protein, 603 aa, unidentified
  REGION 1..603: note = uncultivated organism
  REGION 1..603: note = MG91B-47 Effector
MAKENFVLTLPLRVEKRHADILDKRYELLRQMYNMVQQKLVRQYIYFSQQKAYKACENFEQKREFFNTHPFHFKGIKGKNGELCDIKFPYTYESRSSSSGKRPSGGISDYVSKLWSHKVGADKKLVDFGLNGSIMEELGLHVMKAWEDRLYKFKARRVSFKDYGEINSFGCRYHKGNFAGFTINLEEGFITFNANGRRGQFAEIITLPIDFSREKKGYAEEALKGGVDSLRKLIIVRRCIRGRNKYYLQLTISGEKPKKGRTLGMGNVGIDVGPSTIAISSLQGVHFDKLADKCDNIEHKLYLINRKLDRSRRANNPDNYNPDGTIKRGVRLVWKKDKDVKRYASLKQQRKELFRKQAEIRKQQHLEMANAMLSQGDTFIVENNPIDAWVRRAKETKKTKTGKNRCKKRYGKSVANHAPAMFVSILKNKVLSLGGEFREVNPGYAATQFDFTNGVFTKHEVSERSITLSNGRTHQRDMLSAFNLQHICNELKEYNITQMQADYPIFCRLETAEQERFKEARRNERDALYFVGAFAKELRHSPQEETVANRHNGGVGRKHSNTLCTENAKITGSGLVRESIERVSEAPEECYHFDCQPIILDGL

SEQ ID NO: 198 - protein, 614 aa, unidentified
  REGION 1..614: note = uncultivated organism
  REGION 1..614: note = MG91B-48 Effector
MAKENFVLTLPLKVEKWQADILDKRYELLRQVYNMVQQKLLRQYIYFSQQKAYKECKTFQQKREFFDSHPFYIKIDDKPCEIKFPYTKKNSNTAPNTISNYVSGLGSHRVGQDKTLSNLGINSIILEELGIHIMKAWGDRIYNSKSEHISFKKYGEVNSLVSRYKKKKQTGKVYFPGFTINLEERYITYNTNGRQGNQAKLIKLPIDFSREKKRYAAEALKDGIDSIRKLSVVRRCIRGKNKYYLQLVIDGEKPQKGRTLGRGNVGIDIGPSTIATSSLLGVHFDKLADKCDNIEHELYLIDRKLDRSRRANNPQNYNEDGTYKRGVRLVWKKDSEVKRYAKLKAARKELYRRQAEIRRRQHLEMANAMLQLGDTFIVENNPIDAWARRAKETKKTKSGRTRCKKRYGKSIAHHAPAMFVTMLENKVKSLGGEFYRVDAYGYAATQFDFTNKKFTKHAVNERSITLSNGHTHQRDMLSAFNLQHARLEGEKTKSRDKEKKVESYDIEQMQADYPIFCRLEAEEQKRFRVAWHNEKDALYFVGASGKQLRHQSQEKSAANRHNGGVDRNHCNTRLGKENAEITGSGFARESIARVSQVPDEYHQHGQQSIIIDGL

SEQ ID NO: 199 - protein, 446 aa, unidentified
  REGION 1..446: note = uncultivated organism
  REGION 1..446: note = MG91B-49 Effector
MQAVLYQEGETAVTETKESKNRCVLTLPLLTEPYQEHIIEKRFKILEHLQNSLIAMELRKLKNIQRTRAYRELEQSILQLPKEKRRSLYQQRRQMLEKAGFSRNAFKDDITPMQKHFVGQIATHVAHHVASDVWRAFDKHLYHGAKKVHFKHRGTLSSAANQTAGNAMSMKDDMFIWSGGQSKDKITLRIRVQKPDTDYERDMLQKKVKYFRVVRKWVKTRYKYYLQLTLVGEPAARHRPIGEGRVGIDIGTQSVAIVSAEQAHLWELADRVNANHEKMLHLQRKMDSSRRSMNPENYNEDGTIRRGIRLRWIQSNRYRKLAGQIRELQRKNADIRKYQHVCLANYILTLGSEIYVEDMDYRAMQRRAKKDRKEQTWPIQAKKTFWEVLGQQGTVHVSHHFGHQAAAGNRERASPREQVVFPRQSIRSSVSKVHPQNTRNKKLYAQ

SEQ ID NO: 200 - protein, 476 aa, unidentified
  REGION 1..476: note = uncultivated organism
  REGION 1..476: note = MG91B-50 Effector
MGVCSVLEMRLKTEPWQESLIDKKMECARAVYNATLSETMKNYNRIINTREWQDDQKKVRAFYSGRKAGEKIPQEIKDIFNKRQKQYQEALLTKFGIAKIAQKHARYYNKNVSSNMASVGIGAPVWSSIDKFLYGDGKKVKYKRPGELNSLASNGKSGILLKQDEKGYYLYLSNQKAKAKPLKVYLYDTRTDYDRDMISRKILIVRLVRKAYENGYKYYVQLTVDGAPYERTDEKGEPLCKRGEGKVGLVIDGNNVYAVSDKESRMFKLDENFKEYEKAMEEIETRIEYLRRLNNPNNYEENGTIKRGRLTWNKSCEYKRAAKEKKEMTRKRRETLAITQNVVIKEILEMGNDIYVEKKSYRTKKENYDPDKSDAEYSVKKIRRKKIQNFAPATFLEKLKTKLGDRGALTEISGFGEYFYYCCDSGKSDEERFKNNKVTLDGRVYNAFSYRAVMLKNMENKKLDVEGVKKDLSIIG

SEQ ID NO: 201 - protein, 546 aa, unidentified
  REGION 1..546: note = uncultivated organism
  REGION 1..546: note = MG91B-51 Effector
MANVVYITFGFFRLIMTGLLQPNKNDGIFFQYYQCEVRWAPLAKATTDTCCLTLPLKLEKWHADRLEKRLEIARQIYNTLLRFELKKLHRLEESTEYIDIQNLLKETTDNVERNKLYKQLNKLRQDAGFSEYTFKADIKDFYKHFNNNIGSSVAVHGIAPQVWTAFDRLFYGSGKVVHFKKRGELTSLRGYSVAGKSGGVEIIFRGTYIEWKGLKLPIKLDPKNEYENEMLRQRIKYCRIIKKPGRSKNHWYVQLMLEGKPVIKVNKNTGELLHPVGKGAVGIDIGPQTIAYVSEKEVALKELADQINNIEQEKRVLQRKLDRSRRAMNPGNYEADGTIKRGVKLTRNKSKRYIKTQCELAYLLRRQADIRKRQHNELANHLLSLGDRFYVEDMEWPALAHRAKKTEISEKTGKFKRKKRFGKSIGNKAPSTLIGLLDLKLKSRGLEGVMRVDTKVRASQYNHLTDDYKKKELSERWNNMPDGRRIQRDLYSAFLLQHMNETQTDFDKDALIRDYEQFTVFHDQCIEELRRAPKTLASMGLVRRAS

SEQ ID NO: 202 - protein, 511 aa, unidentified
  REGION 1..511: note = uncultivated organism
  REGION 1..511: note = MG91B-52 Effector
MAKKSATDTCCLTLPLLTEKWQSDRLEKRFEIARQIYNTLLNYELKKLRRLEQSAEYTAIQAAIQKSYEAGKTDKAELKSLFQEREKLRRAAGFSEYDFKTDIKTFYKHFNDNIGSSVAVHGIAPQVWQGFDQLFYGNGKAVHFKRRGDIKSLRGYSVAGKSGGVEIIFRGSYIEWKGLKLSLKLGKNNAYEAEMLQKRVKYCRIVKKAGKHKTHWYVQLMLEGTPAQKCDVNTGELRHPVGRGAVGIDIGPQTIAYAAEAEVALKELADGVKNIEQEKRLLQRKLDRSRRSTNPDNFAADGTIKRGVKLNWHKSKRYIRLQHELAVLQRQQAEVRKRQHTELANHLLSLGDRFYVEDMEWPALTHRAKETAISEKTGRYKRKKRFGKSVANKAPAMLIAILDQKLRSRGLDGVIKVKTSVRASQFNHMTGEYQKKSLAQRWNDMPDGRRIQRDLYSAFLLQHMNAAQDGFDVAALNRDYENFAVLHKRVIEELRTASKTLSSMGITRSVS

SEQ ID NO: 203 - protein, 505 aa, unidentified
  REGION 1..505: note = uncultivated organism
  REGION 1..505: note = MG91B-53 Effector
MGKEEDKEPRCVLTLPLYPEPWQVDIIEKRFHIMERIENSLTAIELRKLKNVERAKKYKDVVAGIKEEKDDKKRSKLWKERNKLLKDAGINQFDFIKDVSLLQKHFASHIAQKVAAAAARTHVWSSFDRVVFGNGRKIHFHKKGDLKSITSGYFGVSMTVKNGYFIWSGGTKDEKDRKVNPITISIRIKQPETYYEKEMLQKQIKYVTIVRKRIKNRYKYYLQFMLVGNPVAKPRTIANGKRVGIDIGTSSAAIVSGKEIKLVELADHIQNNYDKEIQLQRSMDRSRRAMNPQNFNEDGTIKRGIKLIWIESKHYKEMKGQVRELERKNADIRKYQHTCLANYVLSLGTEVYIEQMSFKGLQRKVKETKYDKNGKPKRKKRFGKSIANRAPATFVMILKNKLSGIAGEELHKVNTFTFKASQYDHTNNTYQKKLLGHRWAKLSNGDKIQRDLYSAFLLMNSDTDMQYTDQNKCKRNYKKFKIQHDELIKAMQESSKRYPKSFGIN

SEQ ID NO: 204 - protein, 534 aa, unidentified
  REGION 1..534: note = uncultivated organism
  REGION 1..534: note = MG91B-54 Effector
MGWKSKGRGGKRVKRSSTDTCCLTLPLKLEKWQSDRLEKRFEIGRQIYNTIVHAELKKLRKLERTQQYQEIEQKLENAKQEETKELWKKKRQLLDQNGLSENHFKADMKYYYPHFKDNIASSVAVHGIASQAWTAFEKVLLSKDGKMVHFKKKGELSSLRGYSQAGKSGGIEIIFRGSYVEWKGLKLPLQLSPNNEYETEMLSKRVKYVRILRKDGKTKTHWYAQLALEGKPAVKRDSISGAPKHPVGHGVVGIDIGTRTLAYSADSKVDLIELADRVQNIEQEKMLLQRKLDRSRRATNPENYKPDGSIKQGVKLTWNNSKRYKRIQHQYAMIQHHQADIRKQQHNELANYLLTLGDCFFVEDMDYRALTRKAKKTEISEKTGRYKRKKRFGKSIANKAPSMLIALLDQKCKSLNLEGVKKVDTAVKASQYNHQTDECKAKELKERWNIMPDGERIQRDLYSAFLLQHCRRDLPEYNRKKLQETFGNELYRVFDREALQRDYPKFVEYHRETIQRLSALPKTIASMGIRRSIS

SEQ ID NO: 205 - protein, 505 aa, unidentified
  REGION 1..505: note = uncultivated organism
  REGION 1..505: note = MG91B-55 Effector
MAKATTDTCCLTLPLKLEKWQADRLEKRLEIARQIYNTLLHFELKKLRRLEESKEYIEIQNLLKETTDHAARKGLFKQLNELRRAAGFSEYTFKSDIKDYYKHFKDNIGSNVAVHGIAPQVWAAFDRLFYGNGKSVHFKQRGELTSLRGYSVAGKSGGVEIIFRETLIEWKGLKLPIKLDSKNAYENEMLQQRVKYCRIVKRPGKTASRWYVQLMLEGRPVLKVDRRTGELRHPVGQGAVGIDIGPQTIAYVADGEVALKELADQLGNIEREKRVLQRRLDRSRRATNPNNYAPDGTIKRGTKLTRNKSKRYLKSQRELSFLLHRQAELRKRQHNELANHLLSLGDRFYVEDMEWPALTHRAKETEISEKTGKYKRKKRFGKSVGNKAPAMLLGLLDIKLKSRGLEGVVKVPTSVRASQYNHLTDDYRKKELGERWNTMPDGRRIQRDLYSAFLLQHMNEARSDFDKTALSADYERFVTLHDQCIEELRRAPKTLASMGLVRRAR

SEQ ID NO: 206 - protein, 396 aa, unidentified
  REGION 1..396: note = uncultivated organism
  REGION 1..396: note = MG91B-56 Effector
MQKHFAAHIATHVAHRAASEVWRAFDKFLFGNGKMVHYVRRGTLRSASNQKAGTTMTLENGYFKWNGGRDQNSIAINIRVAKPTTYYEKEMLQKKAKYFRIVRKWGKIGYKYYLQITLAGLPAVKPRLIANEKKVGIDIGPSSIAIVSDKKVELRALADKVQNNHIEKCSLQRSMDRSRRNLNPQNYNEDGTIRRGIRLNWTFSKHYIEMRGKVRMLERKNVDIRKYQHTCLANEVISLGTDVYIEQMSFKGLQRRAKETTYREDGRPKRKKRFGKSIANRAPAMFVEILGRKLTTIVGTELNKVDTFKFRASQYDHTNGSYKKKLLKERWAQLSNGDRIQRDLYSAFLLKNSNADLNSTNVEQCNATYDVFKIMHDDCITKLKSEKKSCLPSFGV

SEQ ID NO: 207 - protein, 534 aa, unidentified
  REGION 1..534: note = uncultivated organism
  REGION 1..534: note = MG91B-57 Effector
MVRKSKGRGGTLVKKSSTDTCCLTLPLKLEKWQSDRLEKRFEIGRQIYNTVVRAELKKLQQVERTPQYREIEQKLKEAKQEEAKELWKTRTQLLSKSGFSEYGFKSDMKYYYQHFKENIGSSVAVHGIASQVWTAFEKVLFSKDGKMVHFKKKGELSSLRGYSQAGKSGGIEIIFRGSYVEWKGLKLPLKLSPDNDYETEMLSKRVKYVRILRKEGKTKPHWYAQLVLEGKPAIKRDPASGAPKHPVGHGVVGIDIGTRTLAYSTDKEVNLLELADRVQNIEQEKRRLQRKMDRSRRAMNPENYNSDGTFKRGVKLTRNKSKRYRRIQHQLAMIQHHQADIRKQQHNELANYLLTLGDCFFVENMSYCDLVHRANKTEISEKTGRYKRKKRFGKSIANKAPAMLITMLKQKCQSRGLKGVKEVDTHVRASQYNHQTDTCIEKALENRWNVMLDGERIQRDLYSAFLLQHCKQGLPEYNRKELKKEFGEELYRVFDREALQRDYPQFVAYHHMTIQRLSELPHTIPSMGIRRIIS

SEQ ID NO: 208 - protein, 490 aa, unidentified
  REGION 1..490: note = uncultivated organism
  REGION 1..490: note = MG91B-58 Effector
MARENENSGCFVVTFPLKTEKWQEDRIDKILTLLTVRYREKQKILLDRYIHLSHSPEFKEAKNAGLKALRDFMNEHKFTKYDVEKVFSDSSLSKSVTTDIMLTHGINSSIMQELSHRAWSAWEKKLYGQGKFIKLDNEVNIFKSRLKGSTITGFEYSFTDMTLSMNATKPKKHTMFTIPFVVNRKSEYEAYAMDCEVRNIAIMRQQIRGKNKYYVQFSFAGTPYNKGRKIGTGIVGIDPGPSKIAIVSDSEIRITTLAPSVNRDERAICRMQRKLDRSRRATNPDNYNDDGTIKRGIKLTFEKSNSYKRTQTKLADTQRKLAAKRKIDHNILANEIIAMGNEFHVENNSFKSMQARSKETKKNDKGKNMSKKRFGKSLANRAPSELLTILKNKVNQYADGDYVDIPSGVACTQFDFTNGEFTKHELKERTITTSDGVVHDRDALAAFNMRFVHKKKIEGKKKIEKDKENFDILAMTEAYRQFCSLEKNIK

SEQ ID NO: 209 - protein, 515 aa, unidentified
  REGION 1..515: note = uncultivated organism
  REGION 1..515: note = MG91B-59 Effector
MELPLQVEKYQEDILNKRYEQLRQLYNYAQGKLLRQFRYFEQMKDYQLCKTFKEKKDFFKNHPFQINGILGSNKQPLDITFDEYGINGFVEKLVQKSAGPNITYADLGITTTILDHLSSSIWRAWDKMLYDYHAKRVSFKKYGELDSFSSRRKVVGGKVYFSGLELHLDTMKLSVKLNGKQGSNAKFITMPILYNPKHEDYELWALKGGLESVKVVKVIRRLVRSQYKYYVQLTIEGEKPQKGRTLGKGNVGIDLGPTTVAVSGENIASIDKLASKCDNIQKEITRLARKIDRSRRANNPENFNEDGTIKRGVRLVWNDSKRYKALRKEMAELQRRQAAIRKQQHIDRANALLKEGDTFIVENNQISAWVRKAKETKVNEKTGKIQKKKRFGKSVANHAPAMFVTILENKVKSLGGEVVKVDTQNAASQYDFTNDSFEKHELNERSVTLSNGDTHQRDMLAAFNLQHLTAEKKQYDREAMAQHYDRFCELEQTEIERYKNKEKKDDRSTIGAGGL

SEQ ID NO: 210 - protein, 552 aa, unidentified
  REGION 1..552: note = uncultivated organism
  REGION 1..552: note = MG91B-60 Effector
MDNNIYVLELPLKVEKWQEDIMIKRLNLLRVLYNNILHKYEIRYNQLIKNKKYREAKTKLYNLDAKKEIKEKIKEIEKDLGKKNAVNDERYISLVNELKTISCTLSESDKKIYKDIKTNLEKQFGFTEYGVYGKVNEGIGWNIAQYAKNCGINPTIIETMCKKNIWKSIEGLLFKGKKIHYKKFNEFNSIYGISASKRLTGIGFDGKYVYWRKSGIKNEKGMYSDIVLPINKTINEYEAQALSNEIAYVGIVRKIIRGKNKFYAQLTLKGILPSKIDKSTNLFRKPAVGRVGLDIGTSTLAIVSDSKVELLELADKAQPNEHKNKIIQRKIDRSIRKNNPNKFNEDGTINLSNRDKWIISKNCQKLKLKLRETYRKLSVVRKLQHEELANHILSLGNECYIETMRWKGLQKRSQNDDVNINGKPKKTKRFGKSIGNRAPSQFMTILSNKLNNYGDKLYLVDTIKCRASQYNHMTKEYNKKPLNERWNVMPNGDKIQRDLYSAFLIQNINNTLNGFNEELLNENYVKFKNKHDEEIKRLQSMSNKKLSSMGIK

SEQ ID NO: 211 - protein, 460 aa, unidentified
  REGION 1..460: note = uncultivated organism
  REGION 1..460: note = MG91B-61 Effector
MSELSVVTIPLKTEIWQEDLLAKRMEIYRSIYNAMLGQVLKRYKKMISDEDYISSKKVIEDTYKITDPKEKNSVKKSQKFKDALEMQRNLFREFGFSEFSFKNESLKYAKHFKDITNTAVVTNTVATQMWRAFDKMIFGDGKMVHFKRYGQLMSLTAPARTGIRIVDENKKTVYAMVPGQDYFCLMSTRKGKNILMPLKIDDRDKYIMQMIQRQIHQVRIVRRDSKGKKRYLVQLIVEGTPYIKLDKNGQEKHPVGAKAVGIYIDTTSITLATHDGIEKIDLTMQSDLMKKKDLQRYMDASRRAMNPDNYNEDGTIKKGRMVNGERVPLTWTYSNGYKKASIELKEIYRKEAEKRMIRANIIANKIIAIGSNIVINDYNFRAASRRKADPELDDKMNYISSKRGGSNIANCAPAGIVAKIDTKLKMRGYEGIKKIRINTDSMIENHREVYAKFLLDKAVK

SEQ ID NO: 212 - protein, 552 aa, unidentified
  REGION 1..552: note = uncultivated organism
  REGION 1..552: note = MG91B-62 Effector
MENSIYVLELPLKVEKWQEDIMVKRFNLLRILYNNILHKYELRYNQLIKIKEYREAKTKLYNLDAKKEIKAKIKAIEKELGKKNAINNENYNLLINELKTISCTLSENDKKTYKDIKSNLEKQFGFTEYGIYGKCNEGIGWNIAQYAKNCGINPTIIERMCKKNVWKSIEGFLFKGKKIHYKKYNEFNSLYGTNVSKVLTGISFDGKCVYWKKSGKKNNKGAYSDIVLPINKTVNEYEAQALSNEIAYVGIIRKIIRGKNKFYAQITLKGCLPSKIDKSTNLFRKPAIGRVGLDIGTSTLAIASNSKVELLELADKAQPNENKIKIIQRKIDRSIRKNNPNKFNENGTINISNKNKWVISKNCQKLKLKLKEMYRKLSVIRKLQHEELANHIISLGNECYIETMRWKGLQKRSQNDDINNNGKPKKKKRFGKSISNRAPSQLTTILSNKLNNYGYKLYLIDTTKCRASQYNHITKEYNKKSLNERWNIMPNGDKIQRDLYSAFLIQNINDTLDGFNEKLLNDNYDNFKVMHNKEIERLRLIPKKKLSSMGIK

SEQ ID NO: 213 - protein, 495 aa, unidentified
  REGION 1..495: note = uncultivated organism
  REGION 1..495: note = MG91B-63 Effector
MSKTDGDRCILTLPLHTEPWQEQIIETRFGIMEHLKNSLIAMEMRKLKNLRRTKTFRDIEKRITETPKEERKALNMQRTAMLRDAGFSEFDFINDITPMQKHFADHIAAQIAHRSASDIWRAFESYLYSSGRDVHFQKRGTLDSVACKKIGNGMNYRNDTFEWSGGRTPNARKLYIPVEKPNTEYEKQMLRKEIKNLRIVRKWMKTRFKYYLQLTLVGQPYQKERRIGNGRVGIDIGTRTIAIASDAAVRLLELADQVDGNHAKKCEIQRRMDASKRATNPENYNADGTIRRPVRGRKMTWHFSNNYMKMQSRVRELERKNADIRKYQHNCLANETLALGTDIFVEPMAYKGLQRRAKETKTDENGRYLKKKRFGKSLANKAPSMFLTILDNKLKCHGKQLLKVDIAQFRASQFDHTNGTYRKKLLSERFAELSNGDRIQRDMYSAFLLMNSNDDLTGPDPERCAATYERFKELHEKETVRIRNKQGRRIASFGI

SEQ ID NO: 214 - protein, 511 aa, unidentified
  REGION 1..511: note = uncultivated organism
  REGION 1..511: note = MG91B-64 Effector
MGKSKTETCCLTLPLKLEKWQEDRLAKRFEIARQIYNTLVHAELKKLNQIERSSEYQTIQDQITNLDRNDPERIKELPSLYKKRTKLLRDAGFTEYGFKSDVKYYYKYFHKNIGSSVAVHGIAPQVWAAFEKKLFKKQGKIVHYKRKGTLRSVKGGSATGKSGGTEIIFRGTYIQWKDLNLPLKQSPDNAYESEMLGYRVKYVRILRKPGKSKDRWYAQLVLEGKPVIKRNPMTGAALHPVGHGAVGLDIGPQTLAYSAADGVGLLELADQVQNIERQKALLQRKLDRSRRASNPDNYNEDGTIKRGVKLTRNQSKRYCKTQRDLAYLQHCQAETRKRQHIALANHLLTLGDRFYVEQMSWAALARRSKKTEVSEKTGKIKRKKRFGKSIANKAPAMLIEILRQKCISLGLPGVVDVPTSIKASQYNHQSKDYIKKNLSERWNDMPDGEKIQRDLYSAFLLQHCTPELNAFDQESLQQDYPQFVQYHHQVMQQLSEMPKTPSSMGIRRRTS

SEQ ID NO: 215 - protein, 490 aa, unidentified
  REGION 1..490: note = uncultivated organism
  REGION 1..490: note = MG91B-65 Effector
MAHKENQKAFVVTFPLRTEKWQEDRIDKIMRMLTTFYNDRQRKLVRRYIYLSHSKAYKEAKSKGVVAFKNYMKENGFSLYGLDAFFKADTKGALYQCGLNSMFLQYLSQCAWSAWDKKLFGNGDFVKTTKVVNIFCSRNLKGKFCGFDYDLSTFTIRIKSTCTKKIICTIPFVVNEKSYYELFALSQKICRIGIVRKLIRSKYKYYVQFTFDGIPYNKGRNIGTGIVGIDPGPSKIAIVGDNKVEILKLAPNIEEDERKTARLKRKLDRSRRATNPHMYKEDGTIIKGQRQTCFSKAYNETRKQLADAQRKLAAKRKIAHNELANTLLEYGDTFKVEANSYKSMQARAKATSVTKSGRIRSKKRYGKSIKNRAPSEFLVILKNKLLYYPKGKYYDVPASYACTQFDFTNESFTEHKISERKIVTSDGIQHNRDTLAAFNIKNSIVNDTSTKKKKITKSKEFFDIKKMKASYNEFCIMEHTSFQYKEIMEL

SEQ ID NO: 216 - protein, 537 aa, unidentified
  REGION 1..537: note = uncultivated organism
  REGION 1..537: note = MG91B-66 Effector
MISDEEYKKLLKQFHKLSDRHILVVETDMPYSDVLKVVILSDKIRKAGNELVSFMRKNYDQLIRTKRYRKLLKLYGSTKDKKKCKDLADQLNEMQKAYNVTWDYCRKSMIPIGKKYGIDAVFALTKAEDVWRGIEKCLYGNGKAVHFSKYGELPCIRAKQINRGIPISVKDDRLQFKLGKTAFGIQVNDRFQQDEVDAVLTYLAESEIVDNKAVNTLMEDAYCIDTYRPCYATLVPKMIRGRYRIYLHLTIEGKAKPKYDKYGNLRHKYGKGVIGADIGTQTVAYTSDTEVGLKNLSERGNSIQTSERKERLLYRAMDRSRRAANPQNYNEDGTVKKGRKTWKYSNHYKKLKAKHSELCRINAVNRQLAINEDANHLRSLGDTFVTEPKNASKLMKRAKETTVNSKGKFNRKKRFGKSIKNRCPSGFQTTVEKKFKTTGGTYIEVPNNYRASQYDHTVDDYIKKKLSDRLYKLQDGTEVQRDWYSSFLLYCYDYKIQKIDKNKCITQFDNCYKKEKALITWIKAHKIKVLNSGIKIA

SEQ ID NO: 217 - protein, 535 aa, unidentified
  REGION 1..535: note = uncultivated organism
  REGION 1..535: note = MG91B-67 Effector
MATENFVLELPLKVEKWQADILNKRYEYLRQIYNYVQRKLIRQYRYFEQMDEFRQCTTYKEKREFFKSHPFYFKDIVGRNGEPFAIKFPYSMSASSGSKMDGFSGYIAKLGKLPAGANKSYSDLGINSFMLERLASNMWAAWNKYLYDRDSKRVSFKKYRDLNTFGSRKANNTTFIGMEIHLDSMEVIIKTNGKLGKYAKYITLPIDYKHSMTEYEMFALKGGFDSIKILNVVRKEIRGREKYYVQLTIEGEKPQKGRMLGKGNVGIDLGPSTIAVSSRQKVSIDKLANKCDDIQRDLYVVSRKMDRSRKANNPQNFNEDGTIKPISRKNGERRYWNDSKRYERLRSERKELLRKQAAIRKLQHIETANALLELGDTFIVENNPVSGWTRKSKETKLNKKGRIQSKKRFGKSVANHAPAMFSTILENKVKSLGGQFKKVDVKNAASRFDFTNGEFTEHKLNERVITLSNGDKHQRDLMAAFNLQHLNLDGDLSKDYDVEMMQRDYPDFCRLEREEMDMYLQGIKKNERTTIGAFK

SEQ ID NO: 218 - protein, 496 aa, unidentified
  REGION 1..496: note = uncultivated organism
  REGION 1..496: note = MG91B-68 Effector
MAKNEQNCCVLTLPLLTEPWQEHIIETRFRIMEQLKNALIAKELGKLNNLKRTREWKRLMKSIQDAKDEKEKKQLFKKRTELLRNVGFSDFSFSNDVTNLKIHKHFAEHINSGIAQRAGKDVWRAFEKYLFSNGKTVHFHRRGTLDSVANRYAREGMMIDGDILTWKGGQSDHQIVLKIRIRRLKNAYEREMLQKTVRNLRIVRKWMKTRYKYYLQITLQGDPASKYRSVGKGRIGIDIGTQTIAWSSPTEVRLAELAAGIKRNHAEKTALQRKMDRSRRTANPQNYNPDGTIRRGVKLRWVKTKHYLRMEGRVRELERKNADIRQYEHICMANHILSLGNEVYIEDMSFKGLQGRAKETKLDKNGRFARKKRFGKSLANRAPSMFVTILENKLKASGGSLHKVNTRTFRASQYDHTADEFKKKTLGQRWNHLKNGDKIQRDLYSAFLLMNAAPSLEEPDKKLCGKTYPQFNRLHDAEMERLRAEEKPHLSSMGIH

SEQ ID NO: 219 - protein, 540 aa, unidentified
  REGION 1..540: note = uncultivated organism
  REGION 1..540: note = MG91B-69 Effector
MMYNTKYQAKRGDPVGEKNSNDTDTCVLTLPLRVEKWQEDCLLKRFEAARRIYNSLLNFELKKLRQLERTVEYRQIQSVISSAYLSNTKDSLECKNAFKRREKLISANGISENGFKSDVKFFYKHFDGLIGSSVAVHCIAVQLWAAFDKYLSGSGKEIHFKRLGDISSLKGYSVKGKSGGLEIIFRGQYVEWTRKKKKLLLPLKIDPDNEYEREMLSRRVKYVRIIKKPGKNRLRWYAQLTLEGKPALKYSAGTGKPIHSVGKGNVGIDIGPQTVAYASDKEVALLELADRVTNIEHQKKKLQRKLDRSRRATNPDNYKPDGTIKRGVKLTRNKSKRYLKLQSKLAYIQHRQAEIRKLQHTCLANHMLTLGDRFFVEKMNWPGLAHRAKETSISEKTGRYKRKKRFGKSLANKAPAMFISILKRKCETLGLPGVVEVDTYNLKASQYNHLSDTYEKKSLSERWNYMPISDGSDSSKTKRIQRDLYSAFLLQHTIPQPCSFDKEALRIDYPNFVTMHDAEIHRLESLPKTITSMGVRRSHS

SEQ ID NO: 220 - protein, 511 aa, unidentified
  REGION 1..511: note = uncultivated organism
  REGION 1..511: note = MG91B-70 Effector
MREDSQNTCCLTLPLKIEKWQADRLIKRFEIARQIYNTLVHAELKKLQKMEASAEYRQIQDRIRELNPSDVRAKKEWKILKDKQEKLWKAWKITEYGFKSDIKAYYKYFNENIGSAVATHDIAPRVWAAFEKKLNDRTGKVKIQYKKAGDIRSLKGFSVSGKSGGTEIIFHKTYIAWKGLTLPLKLSPDNAYEKEMLMRRIKYVRILRKPGKHKDRWYAQLSLEGNPVVKLDPVSGLPRHPIGKGAVGIDIGPQTLAYSAEGGADLVELADQVQCIEQEKSRLQRKMDRSRRASNPDNYAQDGTIKRGVKLTHNKSRRYCKLQKELAYMLYRQAETRKRQHRELINRLLTMGDCFYIEDMHWNSLAHRAKKTEISEKTGRYKRKKRFGKSIANKAPAMLVDMLEQKCKSLELDVVMVNTSLKASQYNHQTGKYEKKTLSQRWNYMPDGEQIQRDLYSAFLLQHVVPEKNAFDNEALCRDYPKFVQYHHEVIERLRSLPKTISSMGIRQSVS

SEQ ID NO: 221 - protein, 483 aa, unidentified
  REGION 1..483: note = uncultivated organism
  REGION 1..483: note = MG91B-71 Effector
MGDKCSVLELKINVRKEQYHRLDVKFEDARAVYNAVLNEALSNYEQITRTKEYRADQAVIKKYYEDKKNGTVKKMPDEVRAIFDKRKKQFQDAKLTKFGLINIAGYHKNHYAKNVSSVIANKIGEMAWTAMNQLIYGNGKLVKFKKPGEMTSIASDNKSGIRLMVDNGKYYLLLSNRAQKAKPMILSIKQPHNEYELDMITRPIKIVRIVRRKNGNRFNYFVQLTVEGTPPQKLKKDGSRVHQIREGKVGVYIKDDSVYAVADDGRIAHWHFCKNQQKYEEKKADYDRELSKLRQLNNPDNYDENGVIKKGSKKWHYSIKYKKILLQRSYLIHKNTESRNITRYVIANEILAMGNDIYILKKDYRTKKEKFDEDNRKSNEQYRQLKDRRKNIQNFAYADLISKIKSKIDSDNSGAVNEVTVSKDLYYYVHDEDKVNKSRFVDGIIAFKNRTCLEESYRALLIMNYDGKNYNRDMVEKGMSWIC

SEQ ID NO: 222 - protein, 508 aa, unidentified
  REGION 1..508: note = uncultivated organism
  REGION 1..508: note = MG91B-72 Effector
MADKECCVLTLPLQTENWQEHIIEKRFAIMEHLKNQLIAKELRRLKNLERTKEYRILIDEINNTPKEKRKALFSKRRKLLKDSGFNRNKFKDDMAVMQKHFEEHIATQIAQEAASDVWRSFDKYLFNNGKYIHFTRKGSLSSINCQKINNGMKYIDGYFIWTCGICSGKRDRRIILKIKVTPPKNEYEKEMLSKKIKYLRIVKKWVKTRYKYYLQFALEGKPVKKDRIVNKGNVGIDIGTQTVAISSADSVKLLELADKIDKNHTKKKILQRKMDRSRRYTNPQNYNEDGTIKKPPKGQRYCWYKSKKYIQLAGKVRELERKNADIRKYQHTCLANWILSLGDTVYVEQMNFSGLQRRAKETKIDKNGKYAKKKRYGKSLANKAPSMFLTILESKLNQYGGQLNKINTYEFKASQYDHTDDTFTKHNRSERWHILSNGDKNQRDLYSAFLIMNSDISLEHCNREKCNETYSNFKVLQDKEIERLYSDIRKGNCKNISSFGFQRNAKAM

SEQ ID NO: 223 - protein, 485 aa, unidentified
  REGION 1..485: note = uncultivated organism
  REGION 1..485: note = MG91B-73 Effector
MANFIVEFPLKTEKYQEDILNKRFEIGRKIYNSLVNITQKRYKEMIKTKKYRKLISSLTGNKKTDKEIWKQINDIRKQYGMSEYSFHEDVKQIQKHFKDNIDSFTAQKIATTLWKSYDKLFFKNGKKIYFKCYGELNSLEGKSNKTGIRLIGDMLIWNKLKIPVMIDYDNYYEYQAMQCDICYNRIVRKYVRNKYKFYVQVVFKGTPPIKADATGKTKHCIGEGDVGLDIGTSTIAVASQSDVKILELADRVQNIENQKQRLLRKIDRSRRFTNFNNYNADGTINKQGNKKLKWNKSSHYIKYQNELKELYRKQADIRKYQHECLANYIISLGNKVYVEKMNFAGLQKRAINTEKNDKGKFKRKKRFGKSLANKAPSMLLTIINRKLAYYGEKLIEIDTINAKASQFNHFDGTYTKKTLSQRWNDFNGIKIQRDMYSAFLIMNIADDLKSFDIDKCNKRFENFYRLHSLEVNRLVGKKNLSSIAI

SEQ ID NO: 224 - protein, 524 aa, unidentified
  REGION 1..524: note = uncultivated organism
  REGION 1..524: note = MG91B-74 Effector
MEEVFVLELPLQVEKYQADILNKRYEQLRQLYNYVQGKLLRQYRYFEQMTEFQACKKIKEKRDFFRSHPFQINETLKGNNTPFKVSFDEYSIKGFVEKLVQKSVGPNMTYADLGFTTYILDHLGTSVWSAWNKKLYDFKAKHISFKKHGELDSFSSRRKVVRGVVYFSGMELHLDTMELSIKLNGKVGKEAKFITMSILHNPKHAEYEMWALKGGLESVKVVKVVRRLVRGQYKYYAQLTIEGEKPQKGRTLGKGNVGIDIGPTTVAVSGENIASIDKLASKCDNIQEEITRLARKIDRSRRANNPENFNEDGTIKRGVRLVWNDSKHYKELRRELAELQRRQAAIRKQQHIDRANELLKEGDTFIVENNQISGWTRKAKETKVNEKTGKIQKKKRFGKSIANHAPAMFVTILENKVKSLGGEVVKVDTQNAASQYDFTNNSFEKHELKERSVILSNGDTHQRDMLAAFNLQHLKYDAPEKKQYDTEAMTQHYDRFCELEQAEIQRYKNKEKKDDRSTIGAGDL

SEQ ID NO: 225 - protein, 511 aa, unidentified
  REGION 1..511: note = uncultivated organism
  REGION 1..511: note = MG91B-75 Effector
MAQRHSTPTCCLTLPLQLEKWQEDRLIKRFEIAGKIYNALLNAELKKLHRLEQLPEYKEIQSQLKAYYQNNLPKTAAFKELNNRRSKLLSEHGFSEYAFKGDIKKFYKHFSSNIGSSVATHCIASQVWSAFEKRLFGGGKFVHFKKSGDIRSLKGFSSSRKSGGVEIMFRGDHIEWKGLLLRLKLDPDNQYESEMLSRRIKYVRIIRKPGKAKWRWYAQLSVEGLPAIKRNDETGEPVHPIGRGAVGIDIGPQTIAYVSQTEVALKELADRVQNIEHEKRLIQRKLDRSRRATNPANYADNGTIKRGVKLTRNKSKRYLKLQMELSFLQHKQAEIRKQQHINLANHLLSLGDCFYVEDMAWSSLARKAKETAVSEKTGKYKRKKRFGKSIANKAPASLISILDQKCTSLGLLGVIKVPTSVRASQYNHITKSYEKKTLSQRWNIMPDGRRIQRDIYSAFLLQHVTPQRDGFSDEALQQDYENFVSLHDSVIQQLRLMPKTIASMGIRRTYS

SEQ ID NO: 226 - protein, 536 aa, unidentified
  REGION 1..536: note = uncultivated organism
  REGION 1..536: note = MG91B-76 Effector
MNEKEYKKSLRQFHKHSDRHILVVETDMSFSDIQKVVALSDKIRKAGNELVGLMIKNYDQLIRTKRYRKVRKLYGATEEKKKRKVLASQLNEMQKQYHVTWDDCRTSMIPIGKKYGIDAVFALTKTEDVWRGIEKCLYANGKTLHFSKYGVLPCIRAKQMNRGIPISVKENQLQFKFGESTFGIQRKDRFQSDEIHAVLDYLAEPEIIDKKAIQTFVEKAYCINTYRPCYATLVPKLIRGKYRVYLHLTIEGKAKPKQDRFGNPRHKFGKGIIGADIGTQTVAYTSDTEVGLKNLSERGNSIQKSERLERLYYRAMDRSRRATNPQNYNEDGTIKKGRKSWKYSNHYKRLKAKHAELCRINARNRQLAINEDANSLRSLGDVLITEPKNASKLMKRAKETTKNKKGKFNKKKRFGKSIKSRCPGGFQSTIEKKFEVSGGRYIEVPGNYRASQYDHTADTYVKKKLSDRMYHLADGTLVQRDWYSSFLLYCYDDKIEEINKTKCRTEFEKHYKKEKALIEWIQANQIKILNSGIKTA

SEQ ID NO: 227 - protein, 535 aa, unidentified
  REGION 1..535: note = uncultivated organism
  REGION 1..535: note = MG91B-77 Effector
MAQETFVLELPLKVEKWQADILDKRYEYLRQIYNYVQKKLIRQYKYFEQMNEFRQCTTYREKREFFRSHPFYYNEVVGRNGEPFAIKFPYSMSESNGAKLDGFNGYISKLGKLPAGANKTYADLGINSFMLERLASNMWSAWNKFLYDRDAKRVAFKKYGELNTFGSRKKNNISFIGMTIHLDTMELVIKTNGRTGRFAEYITLPIGAGKPLTEYEMYALKGGFDSIKILNVVRKQIRGNNKYYLQMTIEGEKPQKGRTLGVGCVGIDLGPSTIAVSSLQKVSIDKLANKCDDIQRDLNRVSRKMDRSRRANNPQNFNEDGTIKPVSRKNGERRVWNDSKRYRKLREERRELLRKQAAIRKAQHIETANALLPLGDTFIVENNPVSSWTRKSKETKINKKGKIQSKKRFGKSVANHAPSMFATILQNKVESLGGEFRKVEVKNAASRFDFTNGEFTEHGLGERVIALSNGDKHQRDLLAAFNLQHLNADGDTTKDYDVATMQEDYPTFCRLEREEMDLYLQGKKKNERTTIGAFK

SEQ ID NO: 228 - protein, 521 aa, unidentified
  REGION 1..521: note = uncultivated organism
  REGION 1..521: note = MG91B-78 Effector
MAKEVFVIEFPLKVEKWQADILNKRYEHLRRLYNYVQSKLLRQYIYFTQMQDYKKCKTLKEKKNFFKEHVFHIKGILGRNKSPLDITFTEYGILGMVTKLSQLSMGGKSYSDIGIGFTILANLADNIWAAWNKRLYDFECERISFKKYGSLDTFGSGKINNKYFCGFDLHLDRMTLDIKINGRTGKQAKFISLPIRYNMKHVDYEMNALAGGFDSIKVLTVVRRMVRGQYKYYLQMTIEGVKPQKGRHLGKGRVGIDIGPTTIAVSSNSTVSIDKLAAKCDNIEKDIQRINRKLDRSRRTMNPDNFNEDGTIKRGVRLIWNNSENYKALRRQKAELQRKQAAVRKMQHIERANSLLSEGDTFIVENNPIKGWAAKAKETKVSEKTGKIQKKKRWGKSVANHSPAMFVTILENKVKSMGGTVIKADVKNAASRYDFTDGSFTKHDLDERGVTLSNGDTHQRDMLAAFNLQHLIYDAEKPKQYDQLRMKSHYPRFCELEQDEIERYRNKVKKDDRCTIAASRL

SEQ ID NO: 229 - protein, 505 aa, unidentified
  REGION 1..505: note = uncultivated organism
  REGION 1..505: note = MG91B-79 Effector
MARSTSDTCCLTLPLKIEKWQADRLEKRLEIARQIYNTLLRFELKKLKQMEAREDYLAIQEQLKETKNDAERKKLYKQIEKIRSETGFSEYSFKTDIKAFYKHFQENIGSSVAVHGIASQVWIAFDKLFHGSGKAVHFKKRGELTSVRGYSVAGKSGGIEIIFRNTFIEWKGLKLPIKLDPSNAYETEMLQKRVKYCRIIKKPGKNKNHWYVQLSLEGKPAVKYHQQTGEILHPVGHGNVGIDIGPQTIAYVADGEIELKELADQVNNIERKKRVLLRKMDRSRRATNPDNYNPDGTIKRGVKLTKNKSKRYIKCQRQLAHLMRQQAETRKRQHNELANHLLSLGDHFYIEDMEWPSLTHRSTKTEISEKTGKFKRKKRFGKSVGNKAPATLTTLLDLKLKSRGLEGVTKVNTMVRASQYNHITDDYQKKKLSDRWNNMPDGRKIQRDLYSAFLLQHMNETHDGFDKAALCRDYEQFTVLHDQCIKRLEQLKKTIASMGIVRKPS

SEQ ID NO: 230 - protein, 609 aa, unidentified
  REGION 1..609: note = uncultivated organism
  REGION 1..609: note = MG91B-80 Effector
MAKENFVLTLPLKVEKCHADILDKRYELLRQTYNMVQRKLLRQYIYFAQQKEYKACENFQQRRDFFASHPFHFKGINSKNGEPADIKFPFTYESRSSNSGKRPSGGISDYVSKLWAHRVGADKTLANLGINSYILEELGLHIMKAWEKRLYDPKSSRVSFKDYGGVNSFGSRYKKGNFAGFTINLEEGFITYNANGRQGRFADLIKLPIDFSREKKDYAAYALKGGIDSLRKLIILRKNIRGRNKYYLQLTISGEKPQKGRALGQGNVGIDIGPSTIATSSLQGVHFDKLADKCDDIEHELYLINRKMARSRYENNRQNYNADKTIKRGVRLVWKKDKDVKRYASLKLKRRELYRRQAEIRKLQHLEMANAMLSQGDTFIVENNPIDAWVRRAKETTKTKTGRNRCKKRYGKSVANHAPAMFVTILKNKVESLGGKFYEVDVRYAATQFDFTNGVKTKHEVSERSITLSNGHIHQRDMLSAFNLQHIKLESIGTDDKEYNIEQMKADYPIFCQLETAEQQRFKEARHNERDAQYFVGAQEKQLRHQPQDESVANRHNGGVGRKHCNTRLEGENAKITGSGLVRESIERVSQVPDEYHPIGDQPIILDGL

SEQ ID NO: 231 - protein, 527 aa, unidentified
  REGION 1..527: note = uncultivated organism
  REGION 1..527: note = MG91B-81 Effector
MNEVFVLELPLQVEKYQADILDKRYEQLRQLYNYVQGKLFRQYRYFEQMKEYQACEKINEKKDFFRTHPFLINETLKGIKTPFMVSFDEYSIKGFIEKLAQKQVGPNMTFADLGFTTYILDHLGASVWAAWDKKLYDFKAKRISFKKYGELDSFSSRRKVVRGVVYFSGMELHLDTMELAIKMNGKVGKEAKFITLKMLHNPKHADYEMWALKGGVEIVKVVKVVRRLVRSQYKYYVQLTIEGEKPQKGRTLGKGNVGIDLGPTTVAVCGENVVSIDKLASKCDNIQEKITRLSRKIDRSRRANNPQNFNEDGTIKRGIKLVWKKDKDLKRYSKLKRELVELRRKQTAIRKQQHIDRANALLKEGDTFIVENNQISSWTMRAKETKVNEKTGRIQKKKRFGKSVANHAPSMFINILENKVKSLGGKVVKVDTKNAASQYDFTDGSFTKHELKERNVTLSNGDTHQRDMLAAFNLQHLKYNEKETKQYDTEAMAADYERFCQLEQEEIQRYKNKEKKDDRGTIGAEKL

SEQ ID NO: 232 - protein, 609 aa, unidentified
  REGION 1..609: note = uncultivated organism
  REGION 1..609: note = MG91B-82 Effector
MAKENFVLTLPLKVEKWQADILDKRYELLRQVYNMVRQKLLRQYIYFSQQKAYKECGKDTKKKGKFFAEHPFFIKIDGKPCEVKFPTWKKSNQTASNSIAKYVSKLSSYKLGQDKTLSDFGINSYILEELGARIMKAWEDRLYKFKAKHISKREYGDVNSFSTRIKKSDNKISYPGFTINLEEGYITYNTNGRRGGFANLIKLPIDFSREKKGYAVEALKGGLESIREISVVRKYIRGKNKYYLQLTIDGEKPQKGRTLGRGNVGIDIGPSTIATSSLMGVHFDKLADKCDDIEHELYLVNRKLDRSRRATNPQNYNTDGTFKTIKNRERRVWNYSNRYNKLKLVRKELFRRQAEIRKQQHLAMANALLSQGDTFIVENNPIDAWVRRAKETSKNKKGNYRCKKRYGKSVAHHAPAMFVSILENKVKSLGGELHKVDVGYAATQFDFTNGEFTKHAVNERSITLSNGHTHQRDMLSAFNLQHARLKTDSRNIEKTVDNYDIEQMNADYPIFCRLETAEQERFKAIRHYERDALFFVGVSNKHLRCQPQEETVANRHNGGVGRKDCNISLGSENAEITGNGLIRESIARVSQVPDECHPFAGKPIVLDGL

SEQ ID NO: 233 - protein, 527 aa, unidentified
  REGION 1..527: note = uncultivated organism
  REGION 1..527: note = MG91B-83 Effector
MEEVFVLELPLQVEKYQADILNKRYEQLRQLYNYVQGKLLRQYKYFEQMKEYEVCKSIREKRDFFRNHPFHITGVLDKSGEPLDITFDEYGIQGFVEKIIHKSVGANTTYADLGFNTLILDYLSASIWSAWDKKLYDFKAKRISFKKYGELDSFSSRRKVVRGVVYFSGMELHLDTMELAIKMNGKVGKEAKFITLKMLHNPKHADYEMWALKGGVDSVKVVKVVRRLVRSQYKYYVQLTIEGEKPQKGRTLGKGNVGIDLGPTTVAISGKNVVSIDKLASKCDNIQEEITRLSRKIDRSRRANNPQNFNEDGTIKRGVKLVWNDSKRYKELRRELAELRRKQAAIRKQQHIDRANALLKEGDTFIVENNQISGWTTRAKETKVNEKTGKIQKKKRFGKSVANHAPSMFVSILENKVKSLGGEVVKVDTKNAASQYDFTDGSFTKHELKERNVTLSNGNTHQRDMLAAFNLQHLKYDEAEAKQYDTDAMAADYERFCQLEQEEIQRYKNKEKKDDRGTIGVEQLLKT

SEQ ID NO: 234 - protein, 525 aa, unidentified
  REGION 1..525: note = uncultivated organism
  REGION 1..525: note = MG91B-84 Effector
MTEVFVLELPLQVEKYQADILNKRYEQLRQLYNYVQGKLLKQYRYFEQTKKFRACKTIKEKRDFFKKHPFHINGILGSNKELLDITFDEYGISGFVTKLGMKNIGSNISYTDVGINSSILAKLSSNIWSSWIKFLYDSKAKRIAFKKYGELNSFGCGKKTNGSRKKIFSGMELNLERMELSIKLNGKVGKSAKFIILPILHNPKHEDYEMMALKGGFDSVKVITVVRRMVRGHYKYYLQMSIEGEKPQKGRTLGKGNVGIDLGPTTVAVSGENIASIDKLAPKCDNIEAAKVKLSRKIDRSRRANNPLNFNKDGTVKRGVRLVWNDSNRYKALRCEMAELHRKQAAIRKQQHIDRANALLKEGDTFIVENNQISGWTRRAKETKVDETTGKIRKKKRFGKSVANHAPSMFVTILENKVKSLGGQVVKVDTQNAASQYDFTNDSFEKHELKERSVTLSNGDVHQRDMLAAFNLQHLKYDETEKKKYNRKAMEQHYERFCELEKEEIRRYKEKEKADDRSTIGTSNF

SEQ ID NO: 235 - protein, 594 aa, unidentified
  REGION 1..594: note = uncultivated organism
  REGION 1..594: note = MG91B-85 Effector
MVDLKKQLCSSASNSENQGVRFQTVVLYTKFVHKSVQLSPESSVFAVALFVRMMYNTKYQAKRGDPVGEKKNNNNNTDTCVLTLPLRVEKWQEDCLLKRFEAARRIYNSLLNFELKKLRQLERTVEYRQIQSVISSAYRSNTKDSPECKAALKRREKLIDENGLTKNGFKNDVKFFYKHFDGLIGSSVAVHCIAVQLWAAFNKYLCGNGKEIHFKRLGDISSLKGYSVKGKSGGCEIIFRGQYVEWTRKKKKLLLPLKIDPDNEYEREMLSRRVKYVRIIKKPGKNRLRWYAQLTLEGKPALKYSAETGKPIHSVGKGDVGIDIGPQTVAYASDKEVALLELADRVTNIEHQKKKLQRKLDRSRRATNPDNYKPDGTIKRGVKLTRNKSKRYLKLQSKLAYIQHRQAEIRKLQHTCLANHMLTLGDRFFVEKMNWPGLAHRAKETSISEKTGRYKRKKRFGKSLANKAPAMFISILKQKCETLGLPGVVEVDTFRLKASQYNHLSDTCEKKSLSERWNYMPISDGSDSSKTKRIQRDLYSAFLLQHTIPQSCSFDKEALRIDYPNFVTMHDAEIHRLESLPKTITSMGVRRSHS

SEQ ID NO: 236 - protein, 527 aa, unidentified
  REGION 1..527: note = uncultivated organism
  REGION 1..527: note = MG91B-86 Effector
MPTENFVLELPLRVEKYQADILDKRYEQLRQLYNYVQGKLLRQYRYFEQMKAYQDCNSIKEKREFFSSHPFHINGIYGRNKDLLDITFSKFGIFGLVTKLGQKSVGSGYTYTDCGISSTILAHMSANIWSAWNKMLNDYHTKRISFKRHGELNSFGSGKIDNEYFPGLVLRLEKMELDIKLNGKTGKNAKFITLSILHNPKHADYEMMALKGGFDSVKVITVVRRMVRGRYKYYLQMSIEGEKPKKGRTLGKGTVGIDLGPSTIAVSGNNIASIDKLASKCDNIEKKIQKLNRKIDRSRRANNPDNYNEDGTYKRGVRLSRNESNRYKALRKEKAELQRRQAAIRKLQHIERANELLKEGDTFVVENNQFKGMAKRSKETKVDEKTGKMRSKKRSGKSIANHAPAMFVTILKNKVESLGGRFEIVNTNNAASQYDFTDGSFTKHELSERNITLSNGDTHQRDMLAAFNLEHLKYEAKDPKKYDSEAMHEHYERFCQLEREEIERYKNKEKKDDRSTISASTIVYDKK

SEQ ID NO: 237 - protein, 579 aa, unidentified
  REGION 1..579: note = uncultivated organism
  REGION 1..579: note = MG91B-87 Effector
MAFGNKNSTPSFVLTLPIKVTKQDEIFLSKKFRVGCTVYNQMVTKTTKMWHQLRKTREYKNLIKAIKAAPANSDERKALLVQRSNLIKQAGFSEGAFHKLVVPYQKAYNVNCDVAQKVASAVWKAWGDFFYGKGKTVHYKRLDDFVTLSGKKNTNGIVFRPANHTVSSLESAKRKAKSSVEKKYFDAYRKLNAKKDEEVVLPDEVKAQMDKEVAAAVAKIKPCIGDGNLRIIYGKHEFLVKLRNPDTQTGRYQQEALKCGVKYCRIVRSWVGTKWKYYAQLVLEGYPPIKCDSNGVAKHPVEQGRIGVDIGTQTIAFCGKDVCDLRVLAPSAVVEARNGLTKEIARVMRQMDRSRRAMNPQYFNENGTVKRLKCKNGHKQIRHWNYSKNYYRLLHKLRDLNRKLAAVRKTEHYILANELLTYGNEFVVEDMNYKALQKRSKKTKINPKTGRARSKKRFGKSIGRCAPAMFISILNQKANRYGGSVIKVSTFETKASQFDHTDDSYTKKKLSQRFAKLSDGTVVQRDMYSAFLLLHLRKNLQSYNKKTIKKDFPLFKKLHDETKERLQNSHEWLPASVGF

SEQ ID NO: 238 - protein, 525 aa, unidentified
  REGION 1..525: note = uncultivated organism
  REGION 1..525: note = MG91B-88 Effector
MNSDTFIMELPLIVEKFQSDELDKRFEILRRIYNMAQNKLERQFCYLSKFKEYQECTTKKAKGEFIKKHPFSFKDILDKSGNPLMITFTKYDIQGYLSKFAKLKIDSQYTYSDMGINTSMLMHMGMAIWSAWEKVIFDSKADKISYKKYGDLKSIGVDWEPKHPAGFILNLRKGELKLNLNNRQGQYARYITMKIRFGKKEHQVWYEGDALNAGEDSIRVLTIVRRKIRGRYNYYLQMTIRNNKPQKGRTLGEGRVAIDLGTSKIAVVAKDKVHLLPLSPSILSVSDELRIIQRQMDRSRRANNPHKYHEDGTVKKGNHDKWVNSKEYIRLRNMKAELERKQAAVRKQDHIALANYLLSLGNEFVIEDNDVQEWAKRKKEEKRRKSDGKNLSKAGLGKQIGNHAPSMFVTILKNKVESLGGTFVKVDNNNRATDYDFTNNTFNEHKLEERYITLGNGNRHQRDTLAAFNLYHLDTAPEKEKCYNLYEMRKHYAQFCEFEKEALLRWKGMTPKREKMKVREHERGL

SEQ ID NO: 239 - protein, 524 aa, unidentified
  REGION 1..524: note = uncultivated organism
  REGION 1..524: note = MG91B-89 Effector
MMKEVFVLELPLLVEKYQADILNKRYEQLRQLYNYVQGKLLRQYRYFEQMVEFQACKTIREKRDFFRSHPFHVNGRLAGDKELPEITFDEYGIKGFVEKLAQKSVGPNMTYADLGFTTYILDHLGANIWSAWDKKLYDYRAKRISFKKFGELDSFSSRRKVVGGKVYFSGMELRLDTMELAIKLNAKQGKNAKFVTLRILHNPKHADYEMWALNGGLDSIKVVKVVRRLVRGQYKYYVQLSIEGEKPQKGRTLGKGNVGIDIGPTTVAVSGENIASIDKLASKCDNIQEEITRLARKIDRSRRANNPENFNEDGTVKRGVRLVWKDSKRYKELRKEMAELQRRQAAIRKQQHIDRANALLKEGDTFIVENNPIKGWAARKKETKIDEKTGKYKSKKGRGKSIKNHAPAMFVTILENKVKSLGGKVERVDTQNAASRFDFTDGSFTDHGYDRKVTLSNGDTHQRDMLAAFNLQHLKYDEAEKKQYDTKAMAQHYDRFCELEQEEIQRYKNKEKKDDRSTIGAGEL

SEQ ID NO: 240 - protein, 512 aa, unidentified
  REGION 1..512: note = uncultivated organism
  REGION 1..512: note = MG91B-90 Effector
MNEVFVLELPLQVEKYQADILNKRYEQLRQLYNYVQGKLLRQYRYFEQMKEYQACKTIKEKRDFFKSHPFQVNESLGGDKSAFEISFNEFSIKGFIEKLVQKSVGANMTYADLGFTTNILDHLGASVWAAWDKKLYDFKAKRISFKKYGELDSFSSRRKVVGGKVYFSGMELHLDTMELAIKMNGKQGNNAKFITLKMQHNPKHAEYEMWALKGGVDSVKVVKVVRRLVRSQYKYYVQLTIEGEKPQKGRTLGMGNVGIDLGPTTVAVSGENVVSIDKLASKCDNIQEEITRLSRKIDRSRRANNPQNFNEDGTIKRGIKLVWNDSKRYKELRRELAELRRKQAAIRKQQHIDRANALLKEGDTFIVENNQISSWTTRAKETKVNEKTGKIQKKKRFGKSVANHAPSMFVSILENKVKSLGGEVVKVDTKNAASQYDFTDGSFTKHELKERNVTLSNGDTHQRDMLAAFNLQHLKYDEKDTKQYDTEAMTADYERFCQFEQEEILLYNNKLN

SEQ ID NO: 241 - protein, 511 aa, unidentified
  REGION 1..511: note = uncultivated organism
  REGION 1..511: note = MG91B-91 Effector
MSKRNTTDTCCLTLPLKLEKWQEDRLSKRFEIARQIYNTLVHAELKKLRQVEQSAEYRQIQVQLREMYQNNTTKTKEFRDIANHRRKLLDAAGFREYDFKSAIKDYYKYFKENIGSNVAIHGIAPLVWTAFENKLYKNGKKVHYKRPGDIYSLRGYSVTGKSGGSEIMFRGTYVEWKGLKLPIKRLPDNDYETAMLEYRVKYVRILRKPGKHKNYWYAQLSLEGKPVIKMNPVSNEPVHPIGSGAVGIDIGPQTIAYASQSEAGLLELASQVQNIERQKGLLQRKMERSRRDMNPENYMPDGTFRRGVKLTKNKSKHYCKLQRELAYLQHRQAETRKRQHIELANHLLSLGDCFYVEDMKWLSLTHRAKETEISEKTGRIKRKKRFGKSIANKAPATLIGILDTKCKSLGIPGVVKVPTIIKASQYNHQSDTYEKKPLGQRWNDMPSGERIQRDLYSAFLLQHLEPDLTGYNKKMLAQDYPQFVEYHNHVINDLSMKPKTLASMGIRRRVS

SEQ ID NO: 242 - protein, 534 aa, unidentified
  REGION 1..534: note = uncultivated organism
  REGION 1..534: note = MG91B-92 Effector
MGWKSKGRGRTRVKRSSTDTCCLTLPLKLEKWQSDRLEKRFEIGRQIYNTVVRAELKKLQQVERTPQYREIEQKLKGAKQEEAKELWKKRTQLLSQSGFSEYGFKSDMKYYYQHFKENIGSSVAVHGIASQVWTAFEKVLFSKDGKMVHFKKKGELSSLRGYSQAGKSGGVEIIFRGSYVEWKGLKLPLKLSPDNDYETEMLSKRVKYVRILRQDGKTKPHWYAQLVLEGKPAIKRDPASGAPKHPIGHGVVGIDIGTRTLAYSADSKVDLIELADRVQNIEQEKMLLQRKLDRSRRATNPGNYNPDGTIKCGVKLTRNKSKRYRKIQHQLAMIQHHQADIRKQQHNELANYLLTLGDCFFVENMSYCDLVHRANKTEISEKTGRYKRKKRFGKSIANKAPAMLITMLKQKCQSRGLKGVKEVDTHVRASQYNHQTDTCIEKALENRWNVMPDGERIQRDLYSAFLLQHCKQGLPEYNRKELKKEFGEELYRVFDREALQRDYPQFVAYHHMTIQRLSELPHTIPSMGIRRIIS

SEQ ID NO: 243 - protein, 499 aa, unidentified
  REGION 1..499: note = uncultivated organism
  REGION 1..499: note = MG91B-93 Effector
MSDLSEKKLGNTVLTLPLYPEKWQIDIIEKRFRIMELLKNALIAKELRRLRNLQRTRVWRRLEDEIRSTDKTERKKLYARRTEMLRTAGFSKYGFYEDIELMQKRYIVHIGAQVAQRASSDVWQAFEKMLYGNGDMVHFHKAGSLYSIANKKSGNCMIFRNGCLEWNGGQSRDAVKLILPSKARLTDYERQMLEKPIKYYRVIRKWMKTRWKYYVQITLAGETVHKDRPVGAGRVGIDIGTRTIAVAAEKAVFLRELADGVDENHRRKCALQRKMDNSRRCTNPENYNPDGTVRRPGKGHRPHWVKSRRYLKMQAMVRNLERKNADIRKYQHTCLANEIIRLGTDVYVEQMSYSGLQRRAKETTYDDKGRTKKKKRFGKSLANKAPASFLLILNQKLSYLEPTGLKRVDTKVFRASQLDHTDQSYHSKKLGERWTVLSDGTKVQRDMYSAFLLMNSNAEMNGVDLEQCNQTFEHFKYMHDRAIETIRNDGKRHPASFGV

SEQ ID NO: 244 - protein, 607 aa, unidentified
  REGION 1..607: note = uncultivated organism
  REGION 1..607: note = MG91B-94 Effector
MAKEEFVLTLPLKVEKWQADILDKRYELLRQVYNMVQQKLVRQYIYFSQQKAYRDCKSFEQKREFFENHPFHIKGVKGRNGELCDVKFPYTYRSRGGPSGKRAPNGISDFVSKLGSRKIGEDKTLADFGINSIILEELGLHIMRAWETRLYKPKSSRVSYREYGDINSFGSRKINTNFTGFTINIEDRYIAFNTNGRRGQFADFIQLPIDFSREKNGYAREALNGGIESLKKLLIVRKCIRGGNKYYLQLTISGEKPQKGRTLGKGNVGIDIGPSTIATSSLKGVHFDKLADKCDNIEHELYLINRKLDRSRRANNPQNYNEDGTIKRGIRLVWKKDKDAKRYATLKQQRKELYRKQAEIRKQQHLEMANAMLSQGDTFIVENNPIDAWVRRAKETTKTKTGKNRCKKRYGKSVANHAPSMLVSILKNKVLSLGGNFYEIDPRNAATQFDFTNGEFTKHEVSERSITLSNGCSHQRDMLSAFNLQHIVLQSKELKEYDIEQMEADYPIFCRLESAEQERFKDARHKERDALYYVGASKKQMRRNPQEKSVANRHNGGVGRKHCNTSLGNENAEITGSGLARESIERVSQVPDEYHPFGCDPIVLDGL

SEQ ID NO: 245 - protein, 524 aa, unidentified
  REGION 1..524: note = uncultivated organism
  REGION 1..524: note = MG91B-95 Effector
MKEVFVLELPLRVEKFQEDILNKRYEQLRQLYNYVQGKLLRQYRYFEQMKEFQACTTLKDKRDFIRTHPFHIYGVYGKNGDLFDITFDEYGINGFVAKLGKKLVGTNTTYADLGITSTILDRLSLAIWASWEKMLYSFHTKRLSFKKYGELDSLSNRRIVVRGKVCFPGMELHLEKMELAIKLNSKTGKNAKFITLPILHNPKHADYEMWALKGGIDSIKVITVVRRLVRGQYKYYLQMSIEGEKPQKGRTLGKGNVGIDIGPTTVAVSGENIASIDKLASKCDNIQEEITRLARKIDRSRRANNPENFNEDGTIKRGVRLVWNDSKRYKALRKEMAELQRRQAAIRKQQHIDRANELLKEGDTFIVENNQISGWTRKAKETKVNEKTGKIQKKKRFGKSVANHAPSMFVTILENKVKSLGGEVIKVDTQNAASQYDFTNNSFEKHELKERSVTLSNGDTHQRDMLAAFNLQHLKYDAQEKKQYDTEAMAQHYERFCELEQAEIMRYKNKEKKDDRSTIGAGDL

SEQ ID NO: 246 - protein, 466 aa, unidentified
  REGION 1..466: note = uncultivated organism
  REGION 1..466: note = MG91B-96 Effector
MGTMSVITIPLKTRVYEEDIINKRMELCRKVYNNMLHDRLKALRKMEHDKDYIDSKNTIFTAYKEQDKKVQKEIKKSQIYKDAQEMQKLKLKEYGFSEYDFKSEILDYIRPFNANITAISAGYTIAAPMWSAFDKYLFSNGQMIHYKRYNTVNSIASDGKAGLKIVDSNKKSTRHPESPFGLSLMYGSNRAGKVICMPLIVDKKDLYMQEMICQNIKIVRLVRRKIKGNYKYYVQLTVDCPPAIKFDRDTGEIKNPINKGNVGLFIDTRKITICSNEGVKQIDLIDKKQLEYASVIADISRYLDTSRRISNPENFNEDGTIKKGIVVEGRRTKLQWTNSNNYIKAKNKKADIERRLAEQRKIRAGEICNLILSLGDNIICNKYNFKAAQMRKKETEVNEKGQYLPKSKAGKAISYSAPATLLTMLDNKLVARGYNPINRVDVDKDMQVTDQSRINMAETLFLQLEN

SEQ ID NO: 247 - protein, 509 aa, unidentified
  REGION 1..509: note = uncultivated organism
  REGION 1..509: note = MG91B-97 Effector
MRKKTTDENFIMELPLTVDKFQSDELYKRFELMRRIYNSAQNKLKRQYEYLSSFKAYKECKTKKEKGDFLKAHPFSFRGVLDKDGEPLKITFSEYDLKSYLSKFAKQKVDSQYTYSDFGINTSMLMHMAMAIWSAWKKVLYDPKAQEVSFKKHGELQSIGVDWEPKHPAGLILDLQKGELKVNLNNRQGQYARYITMKIKFGHKEHQIWYEGDALNAGEDSIRGLTIVRRKIRGKYNYFLQMTIRNNKPQKGRTLGTGRVAIDLGTCKIAVVTKNSVRLIPLAPSISSYSDELRIIQRQMDRSRRANNPQKYHEDGTIKKGNHDKWISSKEYIRLRDIKAELERKQAAVRKQDHIALANYLLSLGNEFVIEDNDVQEWAKRKKEEKKREKDGKNLSKAGLGKQIGNHAPAMFVTILKNKVESLGGSFIKVDTDNGATDYDFTNDEFTHHELEERYITLSDGKRHQRDTLAAFNLYHINTVQNRVYDRLAMQRHYAWFCEKEAESVEQYK

SEQ ID NO: 248 - protein, 491 aa, unidentified
  REGION 1..491: note = uncultivated organism
  REGION 1..491: note = MG91B-98 Effector
MQFTVVIPFKPSNELKNDIDKGLKLLTTRYNTEAKKLKKRYDFWKNTKAYKIAEKEGIDSVSKFWEERSFNGEYGIDKLFKINNFKTHTKPKKNIYKNTIVNASMLQILRANLWRSVDKLIKGKGKNIRYVTMQTIPFKSLRGIKWNQKDSVFSFTHKRGHVITQHIDVKTPYELHAFNNCDMRMVTIKKEIIRGKEKYFFHICFEGTPYNKGRALGTASVGIDPSLENMYACFSNGDMEKLSLIQRIDEEFDIYHKVHVNKIDLLRQKLDRQRRANNPQFFNENGTINREALKQANYTWNDSNGYKKTRNALKDIQRKIALRRKQCHFALANYILSKANKIIVEHNKFAAFAKKSQKDTYKPNGQQYSKRRFGRSINHGAPSYFITILKNKALCWGEKASFKLLSEFNGCTKFDHTNQEFNNEIKRNDKVVTLSNGDKVDRDLHAAMNILFCENGIEQNARRRVEKSADHFNIAIMEEFYNQHKDKMIAI

SEQ ID NO: 249 - protein, 483 aa, unidentified
  REGION 1..483: note = uncultivated organism
  REGION 1..483: note = MG91B-99 Effector
MAHKENQKAFVVTFPLRTERWQEDRIDKMMKMLTTYYNDRQKKLVGRYIYLSHSKAFKEAKGKGVVAFKKYMQENGFSKYDLDAFFKANTKGAIYQIGLNSNNIQYLSQCAWSAWERKLFGNGKFIKTDIDVNIFKSRRKNDSFAGFAYDIGSFTITIKSTYARNNICTIPFIVNRKSEYELFAMSQEIRNIAIVRKVIRGVNKYYVQFTVEGAPYNKGRHLGTGTVGIDPGPSKMAIVADKVVEINKLAPNIEEDEKVTARLKRKLDRSRRATNPHMYNEDGTSKKGQRQTIFSKSYDKTRKQLAEAQRKLTAKRKIAHNELANKILEYGDTFKVETNSFKSMQARSKTTSTTKNGRIRSKKRYGKSIKNRAPSEFLLILKNKLLQYSSGKYYDVPASYACTQFDFTDESFTEHKVSERSIITSDGIQHNRDTLAAFNIKNAIVSDTTSKKRKIVKSKEFFDINVMKETYRAFCEMESKLLA

SEQ ID NO: 250 - protein, 311 aa, unidentified
  REGION 1..311: note = uncultivated organism
  REGION 1..311: note = MG91B-100 Effector
MILLAKNNNNTDTCVLTLPLRVEKWQEDCLLKRFEAARRIYNSLLNFELKKLRQLERTVEYRQIQSVISSAYRSNTKDSPECKDAFKRREKLISANGISEYGFKSDVKFFYKHFDGLIGSNVAVHCIAVQLWAAFDKYLRGSGKEIHFKRLGDISSLKGYSVKGKSGGCEIIFRGQYIEWTRKKKKLLLPLKIDPDNEYEREMLSRRVKYVRIIKKPGKNRLRWYAQLTLEGKPALKYSAETGKPIHSVGKGNVGIDIGPRPLHTLPIKKLLCLNLLTESPISSIKKRSFSASWIAAAAQRIRTTTNRTAQ

SEQ ID NO: 251 - protein, 512 aa, unidentified
  REGION 1..512: note = uncultivated organism
  REGION 1..512: note = MG91B-101 Effector
MKKCTTDTCCLTLPLKLEKWQEDRLAKRFEIARQIYNTLVRAEFKKLNELERTPEYKKILEQIHSLDWKDSGSRQRLNDLYRERNQILKNAKFTEYGFKSDMKFYYKHFSDNIGSCVAVHGIAPQVWAAFEKYLFKPEGKKVHFKRFGDVHSLRGYSKAGESGGAEITFCGTHIRWKNLELPIKLSPDNAYETEMLSRRVKYVRILRKPGKSKDRWYVQLSLEGKPAIKYDRNAGEIKHPVAHGAVGLDIGPQTLAYSAETTADLVELADQVQNIEQEKRRLQRKMDRSRRSTNPGNYNTDGTIRRGVKLTRNKSKAYCKLQRQLAYLQHTQAETRKRQHTELANYLLSLGDCFYVEEMKWSSLAHRAKETAVSEKTGRFKQKKRFGKSIANKAPSTLIGILQQKCESLGLPGIIYVPLSAKASQYNHLTGEYTPKPLSQRWNTMPDAQRVQRDLYSAFLLQHFDSQAGTYNLQSLQRDYPQFLQYHDQSIRRLSEMPKTLSSMGIRKSIRA

SEQ ID NO: 252 - protein, 612 aa, unidentified
  REGION 1..612: note = uncultivated organism
  REGION 1..612: note = MG91B-102 Effector
MTENFVLTLPLKVEKWQADILDRRYELLRQTYNMVQRKLVRQYIYFSQQKAYKACKGDFSQLREFFASHPFHFKGITSKNGELSDIKFPYTYESRSSGSGKRAPNSISDYVSKLWTHRIGQDKTLADLGINGSILEELGLHIMSAWGKRLYDPKASRISFKDYGDVNSFGSRYHKGNFAGFTINLEEGFITYNANGRRGRYADIIKLPIDFSREKNDYAAYALKGGIESFRKLLIVRRCIRGRNKYYLQLNINGEKPQKGRTLGKGNVGIDIGPSTIATSSLQGVHFDKLADKCDNLEHELYLINRKLDRSRRANNPHNYNEDGTIKRGVRLMWKQDKDVKRYASLKQKRRELYRRQAEIRKLQHLEMANAMLSQGDTFIVENNPIDAWVRRAKETTKTKTGRNRCKKRYGKSVANHAPAMFVTILKNKVQSLGGQFLEVDVRYAATQFDFTNGIKTKHEVSERSITLSNGNTHQRDMLSAFNLQHIRQESIGTEDKDYDIEQMQADYPIFCKLETAEQERFKVARHKERDALYFLGASKKQLRRQPQDESVANRHNGGVGRKHCNTRLGSNNQENAKIRGSGHVRESIERVSQVPDGYHLVGNHPIILDGL

SEQ ID NO: 253 - protein, 525 aa, unidentified
  REGION 1..525: note = uncultivated organism
  REGION 1..525: note = MG91B-103 Effector
MKEVFVLELPLQVEKYQEDILNKRYEQLRLLYNYVQRKLLRQYRYFEQMDDFQACKTFKEKRDFFKSHPFHINGIIGSNKELLDITFDDYGINGFVTKLGSKSISDENTYSDMGINSAILAKLSDNIWSSWDKMLYDHKAKRISFKRYGDLNSFGCGKKTNGSGKKVFSGLELHLETMTLAIKLNSKVGKSAKFITLPILHNPKHADYEMMALKGGIDSVKVVKVVRRMVRGRYKYYLQMTIEGEKPKKGRLLGKGNVGIDLGPTTVAVSGESIASIDKLASKCDNIQEEITRLARKIDRSRRANNPENYNEDGTIKCGVHLAWNNSKRYKALRREMAELRRKQAAIRKHQHIDRANALLKEGDTFIVENNQISGWTRKAKETKVNEKTGRIQKKKRFGKSVANHAPAMFVTILENKVKSLGGEVVKVDTQNAASQYDFTNDSFEKHELKERNVTLSNGDTHQRDMLAAFNLQHLKYDEAEKKQYDREAMAQHYERFCELEQEEIMRYKNKVKKDDRSTIGTGNL

SEQ ID NO: 254 - protein, 521 aa, unidentified
  REGION 1..521: note = uncultivated organism
  REGION 1..521: note = MG91B-104 Effector
MNEVFVLELPLQVEKYQADILNKRYEQLRQLYNYVQGKLLRQYKYFEQMKEYQACKAFKEKKDFFRSHPFQINETLKGNETALKVSFDEYSIKGYIEKLVQKSVGQNMNYADLGFTTNILDHLGASIWAAWDKKLYDFKAKRISFKKYGELDSFSSRRKVVGGKVYFSGMELYLDTMELAIKMNGKVGKEAKFITLKMLHNPKHADYEMWALKGGVESVKVVKVVRRLVRSQYKYYVQLTIEGEKPQKGRTLGKGNVGIDLGPTTVAVSGENIVSIDKLASKCDNIQEEITRLSRKIDRSRRANNPQNFNEDGTIKRGIKLVWNDSKRYKELRRELAELRRKQAAIRKQQHIDRANALLKEGDTFIVENNQISGWTTRAKETKVSEKTGKIQKKKRFGKSVANHAPSMFVSILENKVNSLGGQVVKVDTQNAASQYDFTDGSFTKHELKERNVTLSNGDVHQRDMLAAFNLQHLTGEKRQYDVEAMKRDYERFCQLEQEEIQRYKNKEKKDDRGTIGAEKL

SEQ ID NO: 255 - protein, 509 aa, unidentified
  REGION 1..509: note = uncultivated organism
  REGION 1..509: note = MG91B-105 Effector
MARCTTPTCVLTLPLRLEKWQEDRLAKRFEIARKIYNTFLGVKLKELRRLEQTPEYRAIKKELAALYSEKEKNADRIRALYEERNALLKNFTEFKFKKDIKFYYKHFNSNIASKVATHCIASQVWSAFEKYLFGNGKRIHFKRPGEIYSLRGFSATNARSGGSEIIYRGDHIEWNKLRLPLKLDPGNQYEEEMLTNRVKYVRILRRPGKNRDHWYAQLTLEGLPSIKRDDETGDPVHPIGNGPVGIDIGPQTIAYSSAKEAGLLELADRVQNIEDKKCRIQRKMDRSRRATNPGNYNPDGTIKRGVPLTRNKSKRYLRLQKELAWIQHQQAAIRKEQHVALANYLLTLGDRFYIEDMNWQGLARRAKGTAVPEEEKPHKRKKRFGKSIANKAPAALVTILKNKCTSLGLPGVIPINTKVRASQYNHLTDSYQKKELSQRWNEMPDGRKIQRDLYSAFLLQHMNSQLDGFDRAALQRDYESFVSLHDQAILRAAAAPKLISSMGVKRTAN

SEQ ID NO: 256 - protein, 551 aa, unidentified
  REGION 1..551: note = uncultivated organism
  REGION 1..551: note = MG91B-106 Effector
MSNLLFLLENEEKMRYNGVEKQREGEGTRVKRSSTDTCCLTLPLKLEKWQSDRLEKRFEIGRQIYNTIVHAELKKLRKLERTQQYQEIEQKLENAKQEETKELWKKKRQLLDQNGLSENHFKADMKYYYPHFKDNIASSVAVHGIASQAWTAFEKVLLSKDGKMVHFKKKGELSSLRGYSQAGKSGGIEIIFRGSYVEWKGLKLPLQLSPNNEYETEMLSKRVKYVRILRKDGKTKTHWYAQLALEGKPAVKRDSISGAPKHPVGHGVVGIDIGTRTLAYSTDSEVNLLELADRVQNIEQEKRRLQRKMDRSRRAMNPGNYKPDGSIKQGVKLTWNNSKRYKRIQHQYAMIQHHQADIRKQQHNELANYLLTLGDCFFVENMSYCDLVHRANKTEISEKTGRYKRKKRFGKSIANKAPAMLITMLKQKCQSRGLKGVKEVDTHVRASQYNHQTDTCIEKALENRWNVMPDGERIQRDLYSAFLLQHCRRDLPEYNRKELQKEFGEELYRVFDREALQRDYPQFVAYHHMTIQRLSELPHTIPSMGIRRIIS

SEQ ID NO: 257 - protein, 615 aa, unidentified
  REGION 1..615: note = uncultivated organism
  REGION 1..615: note = MG91B-107 Effector
MAKENFVLTLPLRVNKCQADILDKRYELLRQMYNMVQQKLVRQYIYFSQQKAYKACENFEQKREFFNAHPFHFKGIKGKNGELCDIKFPYTYESRSSSSGKRAPSGISDYVSKLGSHKIGQDKTLADFGINSYIMEELGLHIMKAWEDRLYKFKSKRVSFKDYGEINSFGSRYKNGNFAGFTINLKEGFITYNTNGRRGQYADLITLPIDFSREKRGYAAEALKGGVDSLRKLVIVRRCIRGRNKYYLQLTISGEKPQKGRTLGKGNVGIDIGPSTIATSSLYGVHFDELADKCDNIEHELYLLNRKLDRSRRANNPDNYNPDGTIKRGVRLVWKKDKDVKRYASLKQLRKELFRKQAETRKQQHLEMANAMLSQGDTFIVENNPIDAWVRRAKETTKTKTGKNRSKKRYGKSVANHAPAMFVTILENKVKSLGGQFHRIDPGYAATQFDFTNGVFTKHEVSKRSITLSNGRTHQRDMLSAFNLQHARLEAEKAKSRDKEKKVESYDIVQMQADYPIFCRLETAEQERFKDARRNERDALYFVGAFAKKLRHEPQEETVANRHNGGVGRKHCNTLCTENAKITGSGLVRESIERVSEAPEECYHAGDQSIILDGL

SEQ ID NO: 258 - protein, 528 aa, unidentified
  REGION 1..528: note = uncultivated organism
  REGION 1..528: note = MG91B-108 Effector
MKEVFVLELPLRVEKYQADILNKRYELLRQLYNYVQGKLLRQYRYFEQMKDYQICKTFKDKREFIKAHSFHINGVLGRNKELLDISFDEYGIMGLVTKLGQKLIGSGKTYTDMGISSTILANLSDNIWSSWNKKLYDYHADRITFKKYGSLNTFGSGKIVQKKDNEVKEYFPGMVLHLERMEIEIKTNGGLGKKATFITLPILHNPKHADYEMMALAGGSSSIKVLTIVRRLVRGQYKYYLQMSIEGEKPQKGRMLGKGNVGIDLGPTTVAVSGENIACIDKLASKCDNIQAEITKLARKIDRSRRANNPQNYNEDGTIKRGIKLVWYDSKRYKELRRELAELRRKQAAIRKQQHIDRANALLKEGDTFIVENNQISGWTTRAKETKVNEKTGKIQKKKRFGKSVANHAPSMFVTILENKVKSLGGQVVKVDTQNAASQYDFTNDSFTKHELKERNVTLSNGDTHQRDMLAAFNLQHLKYDEKEKKQYDKEAMKAHYEHFCELEQEEIQRYKNKEKKDDRSTIGAEKL

SEQ ID NO: 259 - protein, 609 aa, synthetic construct
  REGION 1..609: note = uncultivated organism
  REGION 1..609: note = MG91B-109 Effector
MAKETFVLSLPLIVEKWQADILDKRYELLRQTYNMVQQKLLRQYIYFAQQKTFKACENFQQKRDFFASHPFHFKGIKGRNGELSDIKFPYTYESRSSSSGKRPSGGISDYVSKLWAHRISQDKTLADFGINGSILEELGLHIMKAWETRLYEPKASRVSFKDYGTVNSFGCRYHKGNFAGFTINLEEGYITYNTNGRRGSLANIIKLPIDYSREKKGYASEALKGGVDSLRKLVIVRKIIRGRNKYYLQLTISGEKPQKGRILGHGNVGIDIGPSTIATSSLQGVHFDKLADKCDNIEHELYLVNRKLDRSRRANNPQNYNEDGTIKRGIRLVWKKDKDAKRYASLKLLRKELYRRQAEIRKLQHLEMANAMLSQGDTFIVENNPIDAWVRRTKETAKTKTGRNRCKKRYGKSVANHAPAMFVSILKNKVLSLGGQFHEVDVRYAATQFDFTNGVKTKHEVGERSITLSNGRTHQRDMLSAFNLQHITLESIGTEDKDYNIEQMYADYPIFCKLESAEQERFKVARKNERDALYFVGVSEKNLRHQHHKETVVNRHNGDVGRKHCNNSLEKENAKITGNGLIRESIERVSEVPDKYHLVGNHPIVLDGL

SEQ ID NO: 260 - protein, 326 aa, unidentified
  REGION 1..326: note = uncultivated organism
  REGION 1..326: note = MG91B-110 Effector
MNEVFVLELPLMVEKYQADILNKRYEQLRQLYNYVQGKLLRQYRYFEQMADYQVCKTIREKRDFLKNHPFNINGVLDRRGKLLDITFDEYGIQGFVEKMIHKSVGNNISYADLGLNTLILDYLAASIWRAWDKKLYDLNAKRISFKKHGELDSFSSRRKVVNGTARFFGLELYLDKMELAVKVNGKQGKNAKFITLKIQHNPKHVDYEMWALKGGVDSVKVVKVVRRLVRSQYKYYVQLTIEGEKPQKGRTLGKGNVGIDLGPTTVAVSGENIVCIDKLASKCDNIQDEITRLSRKIDRSRRANNPQNFNENATIKRGIRLVWNDS

SEQ ID NO: 261 - protein, 502 aa, unidentified
  REGION 1..502: note = uncultivated organism
  REGION 1..502: note = MG91B-111 Effector
MAQEFTLVIPFKPSNELKHDIEQGFKLLTIRYNTELKKLTKRYDYLIKTPEYEIAKREKHEKTFWINKGFTKNGFHKFFRINYFKKTEKSSSSIYDNTIVSSKMLEALKDNLWASFEKLLDGQGKDVHIKNISSMTFKGLCQIKYNPQTQSFDFTHRGRRNIISQKLDIKTDYELHAFANTMRQVTIKKQIIRGKEKYFFHICFQGTPYNKGRELGTNSVGIDPSMLKMFACYSNGEMIEHSLLDKIDEKSGEICDSKYNSDNYEKLEKQVGALQTQINKQRRIKNPQFFDENGKINEEALNKAKLEHPNSSPWEDSKTTIRRKNKLAQIKRNLALHRKECHFRLANYILSKANTINVENNSYKSFQARAKETTYNTNGRPRSKKRFGKSIQKGAPSAFITILKNKAASWGDRVSFSEVGASEACTQFDHTSSEFTKHELKERVVELSNGDRVHRDFHAAMNILFFKNTVTKSKKGTKKDSDHFDIEGMREFYNQHRENMIT

SEQ ID NO: 262 - protein, 534 aa, unidentified
  REGION 1..534: note = uncultivated organism
  REGION 1..534: note = MG91B-112 Effector
MGWKSKGRGGTLVKKSSTDTCCLTLPLKLEKWQSDRLEKRFEIGRQIYNTIVHAELKKLRKLERTQQYQEIEQKLENAKQEETKELWKKKRQLLDQNGLSENHFKADMKYYYPHFKDNIASSVAVHGIASQAWTAFEKVLLSKDGKMVHFKKKGELSSLRGYSQAGKSGGIEIIFRGSYVEWKGLKLPLQLSPNNEYETEMLSKRVKYVRILRKDGKTKTHWYAQLALEGKPAVKRDSISGAPKHPVGHGVVGIDIGTRTLAYSADSKVDLIELADRVQNIEQEKMLLQRKLDRSRRATNPENYKPDGSIKQGVKLTWNNSKRYKRIQHQYAMIQHHQADIRKQQHNELANYLLTLGDCFFVEDMDYRALTRKAKKTEISEKTGRYKRKKRFGKSIANKAPSMLIALLDQKCKSLNLEGVKKVDTAVKASQYNHQTDECKAKELKERWNIMPDGERIQRDLYSAFLLQHCRRDLPEYNRKKLQETFGNELYRVFDREALQRDYPKFVEYHRETIQRLSALPKTIASMGIRRIIS

SEQ ID NO: 263 - protein, 578 aa, unidentified
  REGION 1..578: note = uncultivated organism
  REGION 1..578: note = MG91B-113 Effector
MAFGSKNSTPSFVLTLPLDINLNEQDYLYKEFKKCGIIYNQLVSVTIKMWHQLRKTRKYRELMAAIAKSASDSDEQKTLYKQREKILKEHHFSEDSFHKLVVPYAKHYAIHSHVAQAIATDVWKAWKSFFFSKGKEVHYKRLDDVSSISGKNNATGIVLRPAKLTTSNLASAKRKVQKTVEQRYYKAYGQSDPKDDKKVILPDSVIPQMEAEIAVAVAKVKPAVGNGSLRIVYGKHEFPVVVRNSNTQTGKYQQEALKCGVKFCRITRKWVKIKWKYYAQLVLEGYPPTKCDSNGVIKHPIKQGCVGLDIGTQTLAISGSDICDLRLLAPSARAQAKGLVNEIARTQRAMDRSRRATNPDCYNPDGTIKRLKCQHGHKQKRDWKYSKRYYRLRARLRDLNRRLADIRKMEHNILANELLEHGNEFIVEDMNYKALQKRSKETKVNPKTGRTHTKKRFGKSLSRCAPAIFISILGKKASRYGSSVIKVSTFETKASQFDHTDESYTKKKLSERMAHLSSGEVVQRDLYSAFLLEHIDTESLEYNTEALKSAFPAFLKMHENTKKHLQKEENNLPASVGF

SEQ ID NO: 264 - protein, 534 aa, unidentified
  REGION 1..534: note = uncultivated organism
  REGION 1..534: note = MG91B-114 Effector
MGWKSKGRGGKRVKRSSTDTCCLTLPLKLEKWQSDRLEKRFEIGRQIYNTIVHAELKKLRKLERTQQYQEIEQKLENAKQEETKELWKKKRQLLDQNGLSENHFKADMKYYYPHFKDNIASSVAVHGIASQAWTAFEKVLLSKDGKMVHFKKKGELSSLRGYSQAGKSGGIEIIFRGSYVEWKGLKLPLQLSPNNEYETEMLSKRVKYVRILRKDGKTKTHWYAQLALEGKPAVKRDSISGAPKHPVGHGVVGIDIGTRTLAYSADSKVDLIELADRVQNIEQEKMLLQRKLDRSRRATNPENYKPDGSIKQGVKLTWNNSKRYKRIQHQYAMIQHHQADIRKQQHNELANYLLTLGDCFFVEDMDYRALTRKAKKTEISEKTGRYKRKKRFGKSIANKAPSMLIALLDQKCKSLNLEGVKKVDTAVKASQYNHQTDECKAKELKERWNIMPDGERIQRDLYSAFLLQHCRRDLPEYNRKKLQEAFGNELYRVFDREALQRDYPKFVEYHRKTIQRLSALPKTIASMGIRRIIS

SEQ ID NO: 265 - protein, 567 aa, unidentified
  REGION 1..567: note = uncultivated organism
  REGION 1..567: note = MG91B-115 Effector
MSNLLFLLENVRKMRYNGVEKHWKGEGTRVNNEEKEKEGATCCLTLPLKLEKWQSDHLEKRFEIARQLYNTLVHAELKKKCQVERMPEYQEIMKMIDTASNQKEKWALEKKRKAFLEENGFSRNAFKADMEKYYKHFNDNIGANVALHGITAQVWIAFDKVFVDNYKQKGKVHFKKPGELCSVQGYSQKKSGGKEIMFREDRIIWNQQGNRDRKKVKREFLLKLSPNNPYEAEMLKRHVKFVRILRKDGKTKPHWYAQLVLEGKPAVKRDPISSAPKHPVGHGVVGIDIGTRTLAYSTDKEVNLLELADRVQNIEQEKRRLQRKMDRSRRAMNPENYNPDGTFKRGVKLTRNKSKRYRRIQHQLAMIQHHQADIRKQQHNELANYLLTLGDCFFVENMSYCDLVHRANKTEISEKTGRYKRKKRFGKSIANKAPAMLITMLKQKCQSRGLKGVKEVDTHVRASQYNHQTDTCIEKALENRWNVMPDGERIQRDLYSAFLLQHCKQGLPEYNRKELKKEFGEELYRVFDREALQRDYPQFVAYHHMTIQRLSELPHTIPSMGIRRIIS

SEQ ID NO: 266 - protein, 524 aa, unidentified
  REGION 1..524: note = uncultivated organism
  REGION 1..524: note = MG91B-116 Effector
MTKERTLFMKKMSVLELPLDVQPWQAERLNKRYKIAAMVYNAFRDYEWNKIICIRETKQWREELSDSILRDLYDELKNAKEKESASEEKRINKQIKDIYDRRGKLYKDNGITRFSFLEDIKRFCSPYSTIIPSNMAQLTIAAPLYTAVERCLYGNGDFPRYKRIINSNILKTNGKSGIRLMNDDRGYFLLVSNRRQGAKPMTLPVKVSDSNYNMDMINRDIKIVAIVRKRINGKYKYYTQLTVEGAPFQKVNKDGEYIHQIGNGKVGLNIYKNKVSVASDSGVFEFDLTPDMDAYQSELNELTRCQEAIRRRLNPQNYNDNGTVKKGIYNPETRKTERLHWNSSIKYDEISNKRRELQRVYIERRTIHVNNMIHKILACGDQIIVPDYCFNKDKRKNTKGDEVPINELKKRKDRRIEIQQGAPASFLARLNKVLEQYDREPVSRIRLSEDNFFYCHMSDVDNKDFFKRDHIELPNGTRCSQGLYKAFVLLHYNSDTGEFNHDSICEDFNKFLNMVEAYQSKSTQ

SEQ ID NO: 267 - protein, 510 aa, unidentified
  REGION 1..510: note = uncultivated organism
  REGION 1..510: note = MG91B-117 Effector
MAKATTDTCCLTLPLVLEKWQSDRLAKRFELARQIYNTLLNYELKKLRRLEKTEEYAAGQAKLKELNDRQERDEAARKECHRQLNELRKAAGFSEYDFKTDVKPFYKHFGDNVGSSVAVHGIASQVWQAFERLFYGNGKAVHFKKRGEIRSLRGYSVSGKSGGVEIMFRGSYIEWKGLRLPIKLDADNAYEAEMLQKRVKYCRIVRKEGRRKTRWYVQLMLEGKPALKCDPDTGALRHPTGSGPVGIDIGPQTVAYAAETEAVLQELADGVKDIEREKRTVQRKLDRSRRATNPECFAADGTIKRGVKLNWRKSKRYLALQRELASLQRQQAEIRKRQHTELANHLLSLGDRFYVEDMAWPALTHRAKETAISEKTGRYKRKKRFGKSVGNKAPAMLIGILDQKLRSRGMGGVVKVPTTVRASQFNHVTGEYQKKPLSQRWNEMPDGRRIQRDLYSAFLLQHVNEAQDGFDVAALIRDYENFVTLHDRMIETLKTAPKTLTSMGIWRTAS

SEQ ID NO: 268 - protein, 566 aa, unidentified
  REGION 1..566: note = uncultivated organism
  REGION 1..566: note = MG91B-118 Effector
MGWKSKGRGGTLVDNEEKKEDGATCCLTLPLKLEKWQSDHLEKRFEIARQLYNTLVHAELKRIHRIERMPEYREIERLIEATTDKKEKWALKNQKKQFLRKNGINRNTFTTDMKRLYKHFNDNIGSNVAQHGIVSQVLTAFEKVYIENQSGEVHFKKPGDLRSVQGYSQKESGGKEIMFRNGKIIWTQQENDDRKKSPKYQQRKKRQNYKGVKGIAREFPVVFSKNNPYEAEMLTKHVKFVRILRKDGKTKPHWYAQLVLEGKPAIKRDPASGAPKHPVGHGVVGIDIGTRTLAYSADSKVDLIELADRVQNIEQEKLLLQRKLDRSRRATNPENYKPDGTIKKGVKLTRNKSKRYRKIQHQLAMLQHRQADIRKRQHNELANYLLTLGDCFFVEDMDYRALTRKAKKTEISEKTGRYKRKKRFGKSIANKAPSMLIALLDQKCKSLNLEGVKKVDTAVKASQYNHQTDECKAKELKERWNIMPDGERIQRDLYSAFLLQHCKRGLPEYNRKKLQETFGNELYRVFDREALQRDYPKFVEYHRKTIQRLSALPKTIASMGIRRIIS

SEQ ID NO: 269 - protein, 521 aa, unidentified
  REGION 1..521: note = uncultivated organism
  REGION 1..521: note = MG91B-119 Effector
MAKSKQNKDENKNFVLTLPIKMNVCDEHILDKRYEYLRQIYNIAQRRLLNQFMYFAVTKEYKSIKTIKEKKDFFKTHPFKIKGYDKKITFTEFSIKSYLRNFAKLHVSKDKTYKDFGINTKILDGIFNNIWRAWNKRLYELKVKKIHFKEHDELNNITFYKSNNYFVGLNVDLSNMVIKMNVNGCRMSKAKFITFNLNKKLNDYETFALKGGCDSIKVISIVRKEIRGKKKYYCQFCIEGEKPMKNRILGKGQVGIDLGTSTIAVSSLNGVSIDILAEKCNKIDRETYIIDRKMDRSRRATNPQNYTEDGQIVRNKKGETRKWNYSKHYIKLRSKRKELYRKNAAIRKIEHEMTANKLLSLGNVFVVENNPIKGWQTRAKETTINKKGKYNSKKRYGKSIANHAPSEFITILENKVVSLGGKFEKVDIKNSASRFDFTNETFNEHNVGVRKITLSNGNIHQRDMLASFNLQHLVIPNENLKEYDVEQMKLDYPIYCKLEKQEIALHKNGNKKQYKSVIGKW

SEQ ID NO: 270 - protein, 511 aa, unidentified
  REGION 1..511: note = uncultivated organism
  REGION 1..511: note = MG91B-120 Effector
MKRSGTDTCCLTLPLRLEKWQEDRLAKRFEIARQIYNTMVRAELKKLRKMELSQPYRSIQEQIKALDWKNQNDKQQLRSLYNERDQLLKKNGFTEYGFKSDIKYYYKYFNDNIGSSVAVHGIAPQVWAAFEKMLFRKEGKKVHYKKKDDIHSLRGYSVTGKSGGVEIIFHETYIEWKGLKLPLKLSPHNAYETEMLSRRVKYVRLLRKPGKRKDRWYAQLSLEGKPAIKHNPASGELRHPVGQGAVGLDIGPQTLAYSACCAADLVELADRVQNIEQVKRLLLRKMDRSRRATNPDNYTDDGTIRRGVKLTHNKSKRYQRTQRELRYLQHCQAEMRKRQHIELANHLLTLGDRFYVEDMAWPSLTRRAKETAISEKTGKIKRKKRFGKSIANKAPAALIGILEQKCYSLGLPGVVKVPTSLKASQYNHQSGEYTPKPLSRRWNNMPDGERIQRDLYSAFLLQHYDPQTGNFDPVLLQQDYPQFVQYHHCAIQRLSAMPRTLSSMGIQRSLR

SEQ ID NO: 271 - protein, 511 aa, unidentified
  REGION 1..511: note = uncultivated organism
  REGION 1..511: note = MG91B-121 Effector
MARCTTPTCVLTLPSHLEKWQEDRLAKRFEIAQKIYNALLGAELKKLRRLEQTPKYRAIKKDLAALYGDKEKNADRIRALYKKRNKLLKDNSFTEYGFTVDAKLYYKPFRTNIGSCVAVHCIAPQVWSAFEKYLFGNGKRIHFKRPGEIYSLRGYSSKTSSGVEIIYRGDHIEWNKLRLPLKLDLGNQYEEEMLTNRVKYVRILRRPGKNRDHWYAQLTLEGLPAIKRDDETGDPVHSIGNGPVGIDIGPQTIAYSSSKEAGLLELADRVQNIENKKCRIQRKMDRSRRAANPGNYNPDGTIKRGVPLTRNKSKRYLRLQNELAWIQHQQAAIRKEQHVALANYLLTLGDCFYIEDMNWQGLARRAKETAIPEPEKERKCRRKKRFGKSIANKAPATLVTILKNKCTSLGLPGVIPISTKVRASQYNHLTDSYQKKELSQRWNEMPDGRKIQRDLYSAFLLQHMNSQLDGFDRASLLRDYESFVSLHDQAILRAAAVPKLISSMGIKRTAN

SEQ ID NO: 272 - protein, 528 aa, unidentified
  REGION 1..528: note = uncultivated organism
  REGION 1..528: note = MG91B-122 Effector
MNEVFVLELPLQVENYQADILNKRYEQLRQLYNYVQGKLLRQYRYFEQTKEFQECKTFKAKREFIKAHPFQIKGVLGRNKKLLDIFFDEYGILGLVAKLGQKPIGSRKTYTDMGISSTILANLSDNIWSSWNKKLYDYHANRVSFKKFGELNTFGSGKIIQKKDNEVKEYFPGMVLHLDKMEIDIKTNGALGKKATFITLPILHNPKHADYEMMALAGGSRSIKVLNVVRRLVRGQYKYYLQMTIEGEKPQKGRTLGNGNVGIDLGPTTVAVSGENVVSIDKLASKCDNIQEEITRLSRKIDRSRRANNPQNFNEDGTIKRGIKLVWNDSKRYKELRRELAELRRKQAAIRKQQHIDRANALLKEGDTFIVENNQISGWTTRAKETKVNEKTGKIQKKKRFGKSVANHAPSMFVSILENKVKSLGGEVVKVDTKNAASQYDFTNGSFTKHELKERNVTLSNGDTHQRDMLAAFNLQHLKYDEKDTKQYDTEAMTADYERFCQLEQEEIQRYKNKEKKDDRSTIGAEKL

SEQ ID NO: 273 - protein, 525 aa, unidentified
  REGION 1..525: note = uncultivated organism
  REGION 1..525: note = MG91B-123 Effector
MNEVFVLELPLQVEKYQADLLNKRYEQLRQLYNYVQGKLLRQYRYFEQMKEFQACKTFKEKKDFFRRHPFHIDGILGSNKELYDITFDEYGINGFVGKLGKKQVSAGVTYADLGITSTLLDRLSLAVWAAWDKMLYDYHAQRVSFRKYGELNIFGSRKKTNGSGKKVFSGMELHLEKMEVAIKLNGKVGKAAKFITLPILHNPKHIDYEMWALKGGFDSVTVLTIVRRLVRSQYKYYVQLTIEGEKPQKGRKLGKGNVGIDLGPTTVAVSGVNVVSIDKLASKCDDIQEEITRLSRRIDRSRRANNPQNFNEDGTIKRGIKLVWNDSNRYKELRRELAELRRKQAAIRKQQHIERANALLKEGDTFIVENNPISSWTTRAKETKVNEKTGKIQKKKRFGKSVANHAPSMFVSILENKVKSLGGDLVKVDTKNAASQYDFTNNSFTKHELKERNVTLSNGDTHQRDMLAAFNLQHLQYDEKDDKQYDTEAMTADYERFCQLEQEEILRYKNKVKKDDRSTIGAEKL

SEQ ID NO: 274 - protein, 511 aa, unidentified
  REGION 1..511: note = uncultivated organism
  REGION 1..511: note = MG91B-124 Effector
MEKEFFVIELPLKIEKWEADLINKRYELLRSIYNYAQNKLLRQFKYFEQMTEWKNCKTKKNKSDFFKKHPFYFKGISSKITFSEYDITAFVEKLIKKQVSSNKTYKDFGINSSIAESIGKHIWSAWEKKIYNKGEKISFKKKGKLNSISVRKKSGYFGGIDVDLENMLLRLNINGKIGKKSNKITLPIVFDKPTEYEMYSFKNGIDSINVVTIVRKLIRGNYKYYVQFTIEGKKPQKGRVLGKGKVGVDVGPSTVAVSSINGVQIITLAEKCDGIEHKCKLLERKMDRSRRATNPQNYNDNGTIKKIKNEHLKWVYSKRYSKIRNKKCELQRKQAAIRKLQHNINTNELLKFGDSFIVENNPISAWQHKAKETKINKNGKIQSKKRFGKSIANHAPAMFITILENKVKSLGGELIKVDIKNAASRFDFTNSDFVEHKLNERKITLSNGNTHQRDLLAAFNLQHLNSDDESLKSYNIEEMKKDYPIFCKLEETEIDKFVNGQKKSHKYTVGY

SEQ ID NO: 275 - protein, 524 aa, unidentified
  REGION 1..524: note = uncultivated organism
  REGION 1..524: note = MG91B-125 Effector
MKEVFVLDLPLRVEKYQSDILNKRFEQLRQLYNYVQGKLLRQYRYFEQMKEYQACKTFKEKKDFFRNHPFHINGILGSNKELLDITFDEYGIKGFVEKLVQKGVGTDMTYADLGFTTYILDHLSAGIWSAWDKMLYDYKARRISFKKYGELDSFSSRRKVVQGKVYFSGMELHLDSMMLSVKMNGKVGKAAKFITLPILHNPKHADYEMWALKGGLDSIKVVKVVRRMVRGRYKYYLQMSIEGEKPLKGRKLGKGNVGIDLGPTTVAVSGENIASIDKLASKCDSIETEKAKLGRKIDRSRRANNPQNFNEDGTIKRGIRLEWNDSKRYKELRVEMAELQRRQAAIRKQQHIDRANELLKEGDTFIVENNQISGWTRKAKETKVNENTGKIRKKKRFGKSVANHAPAMFMTILENKVKSLGGKVVKVDTQNAASQYDFTNNSFTKHELNERSVTLSNGDTHQRDMLAAFNLQHLKYDEQEKKQYDTEAMTKHYGRFCELEQAEILRYKNKEKVDDRSTIGAKEL

SEQ ID NO: 276 - protein, 514 aa, unidentified
  REGION 1..514: note = uncultivated organism
  REGION 1..514: note = MG91B-126 Effector
MIVEKYQADILNKRFEQLRQLYNYAQGKLLRQYRYFEQMKEFQACTTFREKKDFLRSHPFHVNGILDRSGDVLNITFDEYGIKGFLEKLIHKPVGSNMTYADLGITTYILDHLSAGIWRAWDKMLYDYHAKRVSFKKYGELDSFSSRRKTSGNKVYFAGMELHLDTMELSVKLNGKQGKDAKFVTMPILHNPKHADYEMWALKGGEDSVKVVKVVRRLVRGQYKYYVQLSIEGEKPQKGRTLGKGTVGIDLGPTTVAVSGENVVSIDKLAAKCDNIQSEITRLARKIDRSRRANNPQNFNEDGTIKRGVRLVWNDSKRYKALRREMAELQRRQAAIRKQQHIDRANELLKEGDTFIVENNQISAWTRKAKETKVNEKTGKIQKKKRFGKSIANHAPSMFVTILENKVNSLGGKVVKVDTQNAASQFDFTDGSFEKHELKERSVTLSNGDTHQRDMLAAFNLQHLKYDDAKKKQYDTDAMGQHYERFCELEREEIERYKKKEKKDDRGTIGAKGL

SEQ ID NO: 277 - protein, 533 aa, unidentified
  REGION 1..533: note = uncultivated organism
  REGION 1..533: note = MG91B-127 Effector
MNEVFVLELPLRVEKYQADILNKRYEQLRQLYNYVQGKLLRQYRYFEQMREFQDCKTFKAKREFIKAHPFQISGISGSNKQPLDITFDEYGIRGFVEKLAQKSAGPNVTYADLGFTTYILDHLSVSIWSAWDKMLYDYHAKRVSFKKYGELDSFSSRRKIVGGKVYFSGMELHLDTMELSVKLNGKQGANAKFITLPILHNPKHADYEMWALKGGLVSVKVVKVIRRMVRSQYKYYVQLTIEGEKPQKGRTLGKGNVGIDLGPTTVAVSGDNIVSIDKLASKCDNIQKEITRLARKIDRSRRANNPQNFNEDGTVKRGIKLVWNDSKRYKELRREMAELQRRQAAIRKQQHIDRANELLKEGDTFIVENNQISGWTRKAKETKVNEKTGKIQKKKRFGKSVANHAPAMFVNILENKVKSLGGEVVKVDTQNAASQYDFTNDSFEKHELKERSITLSNGDTHQRDMLAAFNLQHLKYDDKEKKQYDTEAMKEHYERFCELEQEEIQRYKDKEKKDDRSTIGAEKLHMNDGTVKK

SEQ ID NO: 278 - protein, 497 aa, unidentified
  REGION 1..497: note = uncultivated organism
  REGION 1..497: note = MG91B-128 Effector
MAKNEQNCCVLTLPLLTEPWQEHIIETRFDIVEKLKNTLIGFELRKLKQLERTRAWKRLMKSIRECSDEKQKKSLFKERNTMLKDAGFTEFGFSGDVTKLAVHKHFVPHIGANIAQRSGQDVWRSFEKYLFSNGKAVHFHKRGSLDSIACKKPRGDGMTIDSDVFTWKGGRCDDCVILKIRIRRPKNAYEREMIQKEIRNMRVVRKWIKTRYKYYLQITYQGAPVSKHRPVGKNRVGIDIGTQTIAWSSPTEVRLAELAAGIRRNHAEKTALQRKMDRSRRTANPQNYNPDGTIKRGIKLRWVKTKHYLRMEGRVRELERKNADIRKNEHICLANHILSLGKEVYIEDMNFKGLQGRAKETKVDKNGRFARKKRFGKSLANRAPSMFVTILENKLKSAGGNLYKVNTRTFKASQYDHTADEYRKKTLGQRWNHLKNGDKIQRDLYSAFLLMNAAPTLEEPDKKLCKKTYPQFALLHDAEMERLNAEEKPHLSSMGIR

SEQ ID NO: 279 - protein, 523 aa, unidentified
  REGION 1..523: note = uncultivated organism
  REGION 1..523: note = MG91B-129 Effector
MEKELFILELPLKVEKWQEDLLNKRYEYLRQIYNYAQGKLLKEYNYLSQMADFKNCKTKKAKSEFFNTHPFIIKGFKKPVIFSKMKSKEIEADVYGITDFIEKIVQKKVDNKKTYKDFGINTTMINALANHIWAAWEKVIYGNGKHISFKKYGDINTISIRKKGNFNGLDVDINNMFLAFNINGRKRSNSKKIKLLIDYKKLTDYEIYSLKNGYDSIRVVTIARKLIRGKYKYYVQFTIEGEKPQKGRTLGKGNVGIDVGPSTIAVSSLKGVSIDTLSEKCNDIEHDLYIINRKIDRSRRATNPQNFKEDGQIKPINRREGQKRHWNKSKHYVNLSNKKRELLRKQAAIRKLQHNIKANELLTLGDTFIVENNPISAWVKKSKETKINKKGKFQSKKRFGKSVANHAPAMLITILENKIKSLGGKFIKVDIKNAASRYDFTNNEFKEHGLNERKITLSNGNTHQRDLLAAFNLQHIDLTTEEFKKYDVNEMVKDYPIFCNLEKEEIQRFLDGLKKRHNSTIGI

SEQ ID NO: 280 - protein, 449 aa, unidentified
  REGION 1..449: note = uncultivated organism
  REGION 1..449: note = MG91B-130 Effector
MSDVKGRYILTLPLKTEVYQEHILEKRFRLGEHLYNNFLNFEKKKFYEMTKRKDYRDAQNLIKELTIQINSLDEFKESKQLIQELRKQRKEQYDIIYNIKKMFDWSEFGFTNDMKKYRKYYKANLNSTICDNLSGRCFKAFEKMESELIKDQYRNDEDKVNPCVHYKRKDSLRCLTAKKNSTGIRFSLDEGNRIGTVKWQGLVFSIDLSKCTLYEWQALQSDICYCAVKREKIKGKWKYYIQITLKGHVPDKFDKQTGELKRQLGKGNVGLYFTSTSLTVSTEDGVKTYPLEIKNHEDKKSDLLQKMDASRRATNPDNYNEDGTSKEREEIKGWHFSKAYKKYRNELYEIYRKECEEKKLLQEILANEVIASGDVFNCNKMDFKFLQRNLGRKIQSASPAMLKSTIERKLSYHNVSINEISYTKLNQVFEERNVSKKNLEEVAALIRQF

SEQ ID NO: 281 - protein, 607 aa, unidentified
  REGION 1..607: note = uncultivated organism
  REGION 1..607: note = MG91B-131 Effector
MAKEEFVLTLPLKVEKWQADILDKRYELLRQVYNMVQQKLVRQYIYFSQQKAYRDCKNFEQRRGFFEAHPFHVKSVKGKNGELCDIKFPYTYKSRGGSSGKRAPNGISDYVSKLWSHKIGQNKTLADFGINSYIMEELGLHIMKAWEDRLYKPKSKRVSFKDYGEINSFGSRNKNGNFAGFTINLDNGIITYKTNGRQGQFADLIQLPIDFSREKNGYASEALKGGIESLKKLLIVRRCIRGENKYYLQLTISGEKPQKGRTLGKGNVGIDIGPSTIATSSLNGVYFDKLADKCDNIEHELYLINRKLDRSRRANNPQNYNEDGQIKRGVRLVWKKDKDVKRYATLKLQRKELYRKQAEIRKLQHLEMANAMLSEGDTFIVENNPIDAWVRRTKETTKTKAGKNRCKKRYGKSVANHAPAMFVSILKNKVLSLGGKFYEIDPRNAATQFDFSNGEFTKHEVSERSITLSNGRTHQRDMLSAFNLQHIVLQNKELKEYDIEQMEADYPIFCRLENTELERFKEARHKERDALYFAGASEKQMRRQPQEESVANRHNGGVGRKHCNTSLGGGNAKITGNGLARESIERVSQVPDERHLSGCVPISLDGL

SEQ ID NO: 282 - protein, 528 aa, unidentified
  REGION 1..528: note = uncultivated organism
  REGION 1..528: note = MG91B-132 Effector
MARKKVGEEARCVLTLPLYPEPYQLDVMEKRFRIFEELKRSLFYVELRKLKNLERTRAYRELQQQIRDEYKKQDSQSAKKLKALNARRNEMLKEAGLYQYRFQEDIKPMRAHFETHVYSSMAIAMADDVWKAFEKYLHGSGRQVHGVRRGSLSSVSGKGQWHEDKQSQALLGLDYRDGKLIWSGGGKKYRPDKDGQPGKRKDTREKKHTLEIRVAEPRTEYEREMLKKRICYCRIVRKWMKTRYKYYVQFVLDGNAYAKERVPVPGRVGIDIGPQSAAIVSENRAMLVELAERVDANDKKLHECQRRMDASRRSMNPENFRADGTVKSRRFRWKNSRHYLKLAGQARELQRKNADIRRYAHYCLANEVLRMGSEVYVENMDFRALQKRSKKTEKNEKGRFKRKKRFGKSLANKAPAMFLTILDNKLKSLCGTELHRVDTWSYRASQYDHLSRQYTKKALGERKRVLANGDRLQRDLYSAFLIMNADEERKSVDQQRCDKSYERFKELHDREIERIQNDGKKHLSSFGI

SEQ ID NO: 283 - protein, 521 aa, unidentified
  REGION 1..521: note = uncultivated organism
  REGION 1..521: note = MG91B-133 Effector
MMVTWKEGIPVGKSSTDTCCLTLPLKLEKWQEDRLAKRFEIARQIYNTMVRAELKKLERVKQLPEYRKNQKEIQTLDGKNSADRARLKELYGNQRKILQKQGFTEYGFKADSKDYYKHFIENIGSSVATHGIAPQVWNAFDKMLFQKDGKVVHFKRYGDVHSLKGGSVAHKSGGAEIMFRKTYIEWKGLKLPLKLSPNNAYEAEMLSYRVKYVRILRTRGKTKDRWYAQLSLEGKPAVKYDSSTGKPVHPVGRGAVGLDIGPQTLAYAAANEANLVELADQVQNIEQEKRRLQRKLDRSRRATNPDNYNENGTIRRGVKLTHNKSKRYCKLQKELSWIQHCQAETRKRQHIALANHLLSLGDCFYVEPMAWPSLTHRAKKTEVSEKTGRIKRKKRFGKSVANKAPASLIGILRLKCISLGLTGVVEVPMLLKASQYNHQSKDCAKKSLSQRWNDMPDGNRIQRDLYSAFLLQHYDSQSESFDQETLERDYPQFVRLHNQTIQRLSALPKTPSSMGIRRSVS

SEQ ID NO: 284 - protein, 621 aa, unidentified
  REGION 1..621: note = uncultivated organism
  REGION 1..621: note = MG91B-134 Effector
MRLFDYICIILLMIQSMIEEFVLTLPLKVEPWQADILNNRYEHLRRIYNMVQRKLLRHYIYLSQQNKYRQCKDNDEKRTFWETHHVTIKDFCDKKGNPAILRFPHTYYTRSKASGKRTSHGIPDLLPKLKNRNIGSGLTYASLGINAAILENLGLRIEQAWKKRLYDHKSKHISFKEEGDLNTFGCREKDGFSGFVLDLQKMKLSFNTNGRQGKSAKFIHFPINAGKELTEYEMYALKGGIGSVRKIIVAREYVRGRYKYYVQLTIKGEKPQKNRTLGKGTVGIDVGPSTVAVSSLHGVSLDKLADKCDNIEHDLWRINRKMDRSKRDTNPDFFNEDKTYKKPEKGERRKWTFSNRYKRLKLKRRELYRKQAAIRKRQHIEMANALLSQGDEFIVENNPIDSWTKRAKETKRGKNGKIRCKRRFGKSVAHHAPAMFVSILQNKVRSLGGTFHKVEIKNAATQFDFTNGEFTKHELNERTVTLSNGRTHQRDMLSAFNLQHLRTNSEELKDYDIEQMEADYPVFCKLEEKGIAQFKEKRQQERDVAYFTGVSHKTLKFSSANRHNGGVGRGNCNYHAGSKRNVAKTGNKSSSKENCESMETVSLVPNEYHHSNHQPFRLDGL

SEQ ID NO: 285 - protein, 495 aa, unidentified
  REGION 1..495: note = uncultivated organism
  REGION 1..495: note = MG91B-135 Effector
MPNTDSNRCILTLPLMTEPWQEHLIETRFDIMEHLKNSLIALELRKLKNLQRTRAYKELEKKIFEKPKEERKPLYKERSAMLRTAGFSEFDFINDMTPMQKHFADHIAAQISHRSASDVWRAFESYLFSSGREVHFQKRGTLDSVACKKAGNGMNYRNDTFEWSGGRTPNAKRLCIRVENPQTEYEKHMLEKEVKNMRIIRKWMKTRYKYYLQLTLVGDPYQKPRKIGEGRVGIDIGTRTIAIASETTVRLLELADRIDGNHKRKCELQRKMDASKRATNPDNFNSDGTIRRRSKGKKMTWHFSKHYLRMRDQVRELERKNADIRTYQHNCLANEVLTLGTDVFVEPMSYKGLQHRAKETKVDEKGRYLKKKRFGKSLANKAPSKFLMILENKLACKGKALNYVDINRFRASQFDHTNGTYRKKQLSERFATLSTGEHVQRDMYSAFLLMNSNDSLNAPDIERCGQTFERFKELHAIEVERIRQSPYRRIQSFGI

SEQ ID NO: 286 - protein, 524 aa, unidentified
  REGION 1..524: note = uncultivated organism
  REGION 1..524: note = MG91B-136 Effector
MEEVFVLELPLQVEKYQADILNKRYEQLRQLYNYVQGKLLRQYRYFEQMSEFQGCKTIREKRDFFRSHPFHIKGKLAGNKELPEITFDEYGINGFVEKLAQKSAGSNTTYADLGFTTYILDHLSTSIWAAWEKMLYDYRAKRISFKKYGELDSFSSRRKVVGGKVYFSGMELHLDTMELAIKLNAKQGKNAKFVTLRILHNPKHADYEMWALKGGLESIKVVKVVRRLVRGQYKYYLQMSIEGEKPKKGRTLGKGNVGIDLGPTTVAVSGENIASIDKLASKCDNIQEEITRLARKIDRSRRANNPQNFNEDGTIKRGVRLVWNDSKRYKALRREMAELQRRQAAIRKQQHIERANELLKEGDTFIVENNQISGWTRKAKETKVNEKTGKIQKKKRFGKSVANHAPSMFVTILENKVKSLGGEVIKVDTQNAASQYDFTNNSFEKHELKERSVTLSNGDTHQRDMLAAFNLQHLKYDKQEKKQYDTEAMAQHYERFCELEQEEIMRYKNKEKKDDRSTIGAGEL

SEQ ID NO: 287 - protein, 495 aa, unidentified
  REGION 1..495: note = uncultivated organism
  REGION 1..495: note = MG91B-137 Effector
MPFKPSNELQHDIEQGFKLLTIRYNTELKKLKKRYDYLIKTQEYEIAKREKHEKTFWEDNNFTKYDFYEFFFRINSFKRAQHPQQSIYDNTIVSSTMLRALGDHLLSSFDKLLYQNGKDVHPKKISSMTFRGLVQIRYNSQTQSFEFTHRGRRNIISQKVDIKTDYEIYAFNNPIKEVTIKKQIIRGKEKYFFHICFRGTPYNKGRELGTNSVGIDPSMLKMFACYSNGEMIEHSLLDKIDEKSGEICDSKYNSDKYEKLEKKIGVLQTQINRRRRIENPKFFKENGEINEEALNQARLEHPNSDPWKYSKATIKMRNKVAQIKRNLALHRKECHFRLANYILSKANTIKVENNSYKSFQARAKETTYNANGRPRSKKRFGKSIQKGAPSAFITILKNKAASWGDRVSFSEVGASEACTQFDHTNDEFTKHELKERVVELSNGDRVHRDFHAAMNILFFKNTVTKSKKGTKKDSDHFDIEGMREFYNQHRENMIT

SEQ ID NO: 288 - protein, 605 aa, unidentified
  REGION 1..605: note = uncultivated organism
  REGION 1..605: note = MG91B-138 Effector
MVEEFVLTLPLKVEPWQADILNNRYEHLRRMYNMVQRKLLRHYIYLSQQKAYRQCKDNDEKRTFWENQPITFKDLCDKNGNPAIVRFPHTYYTRSKASGKRTSNGIPDLLPKFKNRNIGSGLTYASLGINAAILENLGLRIEQAWKKRLYDPKSKHISFKDEGDLNTFGCREKDGFSGFVLDLPRMMLSFNINGKQKSQAKFIHLPINAGKELTEYEMYALKGGLDSIRKIIIAREYVRGRYKYYVQLTIKGEKPQKNRTLGNGSVGIDVGPSTVAISSLQGVTLDKLADKCDDIEHDLWRINRKMDRSKRKTNPDFFNDDKTYKKRKRGERRIWHFSKRYKRLKLERRELYRKQAAIRKQQHIDMANALLSQGDEFIVENNPIDSWAKCAKETKKNKNGKNRCKRRFGKSIAHHAPAMFISILHNKVRSLGGSFSKVEIKNAATQFDFTNGEFTKHELNERIVTLSDGRTHQRDMLSAFNLQHLRPESEEIKDYDIEQMEVDYPIFCKLEEQGIAIFKEKRPQDRDVAYFVGTSHKTLKISSANRHNGGVGRKNCNHHAGKRKNVAKAVTKSSSKKNCESLESVSVVPNEYHHSDNQHLRLDRL

SEQ ID NO: 289 - protein, 495 aa, unidentified
  REGION 1..495: note = uncultivated organism
  REGION 1..495: note = MG91B-139 Effector
MAGKSDKARCVLTLPLLTEPWQEHIIEKRFRIMEHLKNSLIALELRKLKNIQRTKQYRQLKAEISNAPKENRKTLDKKRKELLINAGFSERAFKDDMTNMQKHFVEHISTHAAHKSASDVWRSFDKVLFHTGRKIHFIRRGSLSSIACQTDGNGMRFRNGMLHWNGGRCKNKVKLELRVACPKTPYECAMLEKKIKFERIIRKWVKTRYKYYVQFTLVGNAVPKPRKTGTGRVGIDIGPSSIAIASSQEVRLMELADKINQNHLRKVALQQKMDRSRRATNPDNFNENKTIRRGVKLSWHYSKHYQKLRGQVRELERKNADIRKYQHTCLANYILSLGTEVYIEQMNFKGLQARAKETTYNKNGRANRKKRFGKSIANRAPAMLVTILETKLKSYAGTELHKVNTWSYRASQYDHITDTYTKKLLRQRWAELGNGDRIQRDLYSAFLLMNSQPSLQSTDRERCSKTYSQFKSLHDQEIYRLQQTPERHLSSFGIA

SEQ ID NO: 290 - protein, 528 aa, unidentified
  REGION 1..528: note = uncultivated organism
  REGION 1..528: note = MG91B-140 Effector
MNEVFVLELPLQIEKYQADALNKRYEQLRQLYNYVQGKLLRQYRYFEQMKEYQACKKIKEKRFFFRSHPFQINETLKGNKIPFKVSFDEYSIKGYIEKLVQKSVGPNMTYADLGFTTNILDHLGASVWAAWDKKLYDLKAKRISFKKYGELDSFSSRRKVVRGVVYFSGMELHLDSMELAIKMNGKVGKDAKFITLKMLHNPKHADYELWALKGGVDSVKVVKVVRRLVRSQYKYYVQLTIEGEKPQKGRTLGKGNVGIDLGPTTVAVSGVNVVSIDKLASKCDNIQEEITQLSRKIDRSRRANNPQNFNEDGTIKRGIKLVWSDSKRYKELRRELAELRRKQAVIRKQQHIERANALLKEGDTFIVENNPISSWTTRAKETKVNEKTGKIQKKKRFGKSVANHAPSMFVSILENKVKSLGGRFERVSTQNAASQYDFTTDTFKTGEEKHKLNERAITLSNGDTHQRDMLAAFNLQHLKYDEKDAKQYDTEAMAADYERFCQLEQEEILRYKNKVKKDDRGTIGAEKL

SEQ ID NO: 291 - protein, 495 aa, unidentified
  REGION 1..495: note = uncultivated organism
  REGION 1..495: note = MG91B-141 Effector
MQKENKNGCCILQLRLKPEPWQVDVIETRFRILEHLKNSLTAYELRKLKNLERTREYRAILEALKIADEKTKKNLYKKRQKMLTDAGFSEFRFKDDITPMQKHFVEHISTHVAHRAASEVWRAFDKFLFGSGKTVHFVRRGTLKSAANQKAGTSMAMKNGYFEWNGGRSSNTISLKVRIAKPETYYEKEMLQKKAKYFRIVRKWGKTGYKYYLQITLEGQPVLKPRAIANGKKVGIDIGPSSIAIVSDKKVELRQLADHVQNNYEEKCLLQRSMDRSRRNLNTQNYNEDGTIRRGIRLEWKLSKHYMEMRGKVRMLERKNADIRKYQHTCLANEILSLGTNVYIEQMSFKGLQRKAKETTYREDGRPKRKKRFGKSIANRAPAMFVEILNKKLETIVGVELNKVDTFQFKASQYDHTNGSYKKKLLKERWAQLSNGDRIQRDLYSAFLLMNSNNDMNSADVDKCNATYIEFKKLHDKCIDKLKTDGGIRLPSFGI

SEQ ID NO: 292 - protein, 523 aa, unidentified
  REGION 1..523: note = uncultivated organism
  REGION 1..523: note = MG91C-1 Effector
MEKELFILELPLKVEKWQEDLLNKRYEYLRQIYNYAQGKLLKEYNYLSQMADFKNCKTKKAKSEFFNTHPFIIKGFKKPVIFSKMKSKEIEADVYGITDFIEKIVQKKVDNQKTYKDFGINTTMINALANHIWAAWEKVIYGNGKHISFKKYGDINTISIRKKGNFNGLDVDINNMFLAFNINGRKRSNSKKIKLLIDYKKLTDYEIYSLKNGYDSIRVVTIARKLIRGKYKYYVQFTIEGEKPQKGRTLGKGNVGIDVGPSTIAVSSLKGVSIDTLSEKCNDIEHDLYIINRKIDRSRRATNPQNFKEDGQIKPINRREGQKRHWNKSKHYVNLSNKKRELLRKQAAIRKLQHNIKANELLTLGDTFIVENNPISAWVKKSKETKINKKGKFQSKKRFGKSVANHAPAMLITILENKIKSLGGKFIKVDIKNAASRYDFTNNEFKEHGLNERKITLSNGNTHQRDLLAAFNLQHIDLTTEEFKKYDVNEMVKDYPIFCNLEKEEIQRFLDGLKKRHNSTIGI

SEQ ID NO: 293 - protein, 544 aa, unidentified
  REGION 1..544: note = uncultivated organism
  REGION 1..544: note = MG91C-2 Effector
ISLIVFNIKYLVVSKFWFTFANEIRKKNRCMEKEMFVVELPLKVEKWQSDVLNKRYELIAKIYNYAQKKLLRQYIYLVQMDEYKKCKTKKEKSIFFKNHPFYFKDITSKITFSEYDVTAFVEKIIKKNVSSDKTYKDFGINTSITECIGKHIWSAWEKKIYGNGKKVSLKNKGELNTISIRKKSEYFGGLDIDIEHMILSFNINGKTKSKAKKIILPINVKNLTEYEMYALKDGYDSINVVTIIRKMIRGNYKYYVQFTIEGEKPQKGRTLGNGNVGIDIGPSTVAISSLNGVTIDTLAEKCDNIESKISNIERKMDRSRRATNPQNFTEDGQIKRIDRKKGEKRVWKYSKRYVKLRNKKRELLRKQTVIRKLQHNIKANEILSLGDTFIVENNPISAWAKRANGVRKNKNGKIQTNKRLGKSIGNHAPAMFVTILQNKVESLKGKFVQVDLKNAASRFDFTNGDFTEHNLDERKITLSNGNTHQRDMLAAYNLQHIDLTNDYLKKYNVDEMIEDYPIFCKLEKEEIKRFIDKMKISNKSNIGL

SEQ ID NO: 294 - protein, 523 aa, unidentified; identical to SEQ ID NO: 279
  REGION 1..523: note = uncultivated organism
  REGION 1..523: note = MG91C-3 Effector

SEQ ID NO: 295 - protein, 509 aa, unidentified
  REGION 1..509: note = uncultivated organism
  REGION 1..509: note = MG91C-4 Effector
MNKRIKEDGKENFILCLPLQVTKQQADILNKRLSICCGIYNNFQKKMLRKFNYVKQMKAYKECKTLSERRKFLNGYTIQWKNTKGKLYDLKPFTEFGFKSYIAVFNETFSNNGINSKLLQYIASNAWSAWDKVLYGKGKRISFKKRDEFNSYTIGISSNNTFNGIDISKINENKIGVNLNGGRGKNAKVIYIPYKINTDYERECFLNDIRAIWLKRVFIRGKWKYFITFSFEGKKPVKNRKLGKGKVGIDIGPSTIAISSDDVVYMDVLAKNVNSIEDKKRLLQRKIDRSKRKTNPLQFKEDGSIRRYKKGERPKWIISNHCKDYYVKLKELYRKQSVYRKNSHIELSNMLLNLGNDFIVENNPVEAWMRKSKEITFNKKGKINSRKRFGKSIANHAPSMFITILENKVKSLGGSLTKADIKNAASQFDFTNETFTKHELKERRINLSNGNTHCRDSIAAFNLQHLIGDKEYDVSTMKEKYNKFCRMEQEEIKRHQMFDEKISSNFGIN

SEQ ID NO: 296 - protein, 523 aa, unidentified
  REGION 1..523: note = uncultivated organism
  REGION 1..523: note = MG91C-5 Effector
MKSEKKNRCMEKEMFVVELPLKVEKWQSDVLNKRYELIAKIYNYAQKKLLRQYIYLVQMDEYKKCKTKKEKSIFFKNHPFYFKDITSKITFSEYDVTAFVEKIIKKNVSSDKTYKDFGINTSITECIGKHIWSAWEKKIYGNGKKVSLKNKGELNTISIRKKSEYFGGLDIDIEHMILSFNINGKTKSKAKKIILPINVKNLTEYEMYALKDGYDSINVVTIIRKMIRGNYKYYVQFTIEGEKPQKGRTLGNGNVGIDIGPSTVAISSLNGVTIDTLAEKCDNIESKISNIERKMDRSRRATNPQNFTEYGQIKRIDRKKGEKRVWKYSKRYVKLRNKKRELLRKQTVIRKLQHNIKANEILSLGDTFIVENNPISAWAKRANGVRKNKNGKIQTNKRFGKSIGNHAPAMFVTILQNKVESLKGKFVQVDLKNAASRFDFTNGDFTEHNLDERKITLSNGNTHQRDMLAAFNLQHIDLTNDDLKKYNVDEMIEDYPIFCKLEKEEIKRFIDKMKISNKSNIGL

SEQ ID NO: 297 - protein, 509 aa, unidentified
  REGION 1..509: note = uncultivated organism
  REGION 1..509: note = MG91C-6 Effector
MNKRIKEDGKENFILCLPLQVTKQQADILNKRLSICCGIYNNFQKKMLRKFNYVKQMKAYKECKTLSERRKFLNGYTIQWKNTKGKLYDLKPFTEFGFKSYIAVFNETFSNNGINSKLLQYIASNAWSAWDKVLYGKGKRISFKKRDEFNSYTIGISSNNTFNGIDISKINENKIGVNLNGGRGKNAKVIYIPYKINTDYERECFLNDIRAIGLKRVFIRGKWKYFITFSFEGKKPVKNRKLGKGKVGIDIGPSTIAISSDDVVYMDVLAKNVNSIEDKKRLLQRKIDRSKRKTNPLQFKEDGSIRRYKKGERPKWIISNHCKDYYVKLKELYRKQSVYRKNSHIELSNMLLNLGNDFIVENNPVEAWMRKSKEITFNKKGKINSRKRFGKSIANHAPSMFITILENKVKSLGGSLTKADIKNAASQFDFTNETFTKHELKERRINLSNGNTHCRDSIAAFNLQHLIGDKEYDVSTMKEKYNKFCRMEQEEIKRHQMFDEKISSNFGIN

SEQ ID NO: 298 - protein, 521 aa, unidentified; identical to SEQ ID NO: 228
  REGION 1..521: note = uncultivated organism
  REGION 1..521: note = MG91C-7 Effector

SEQ ID NO: 299 - protein, 511 aa, unidentified; identical to SEQ ID NO: 274
  REGION 1..511: note = uncultivated organism
  REGION 1..511: note = MG91C-8 Effector

SEQ ID NO: 300 - protein, 601 aa, unidentified
  REGION 1..601: note = uncultivated organism
  REGION 1..601: note = MG91C-9 Effector
MIEEFVLTLPLKVEKWQADILNNRYEHLRRIYNMVQRKLLRQYIYYSQQSEYRKCKSNDEKRDFWENHPFTIKGIYDQKGNSSIVKFPYTYSSRSKNSGKRASNGISDLLPKLKHYNIGGGKTYGSLGLNASILENLGLSVQQAWEKRIYNSKSKHISFKKEDELNTFGCREKDGFSGFVLDLSKMELSFNTNGLHGKKAEFIHLPINPNKKFTQYEVEALKGGPESVRKIVISREYIRGAYKYYVQLTIRGEKPQKNRTLGKGTVGIDVGPSTVAVSSLYGVALNKLADKCDGIEHDLWLVNRKMDRSRRATNPDFFAEDGTYIKRKKGERRIWNFSNRYKNLKSVRRELYRRQAVVRKQQHIAMANEMLSQGDTFIVENNPVDSWTRRAKETKKNKKGKMRCKRRFGKSVAHHAPAMFVSILQNKVRSLGGEFHKVDIKIAATQFDFTNGEYKKHELKERTVTLADGKTHQRDMMAAFNLQHFRADSSEDYDIEQMILDYPVFCKLEEQGIANYIESKGEEKDTAYFAGKSSKELKRSSNRHNGGVERKHCNIHERKGDAAESGKKTLFGGISESLESVPDVPNECHHLGHQHLRLDEP

SEQ ID NO: 301 - protein, 621 aa, unidentified; identical to SEQ ID NO: 284
  REGION 1..621: note = MG91C-10 Effector
  REGION 1..621: note = uncultivated organism

SEQ ID NO: 302 - protein, 509 aa, unidentified
  REGION 1..509: note = MG91C-11 Effector
  REGION 1..509: note = uncultivated organism
MNKRIKEDGKENFILCLPLQVTKQQADILNKRLSICCGIYNNFQKKMLRKFNYVKQMKAYKECKTLSERRKFLNTYTIQWKNTKGKLYDLKPFTEFGFKSYIAVFNETFSNNGINSKLLQYIASNAWSAWDKVLYGKGKRISFKKKDEFNSYTIGISSNNTFNGIDISKINENKIGVNLNGGRGKNAKVIYIPYKINTDYERECFLNDIRAIGLKRVFIRGKWKYFITFSFEGKKPVKNRKLGKGKVGIDIGPSTIAISSDDVVYMDVLAKNVNSIEDKKRLLQRKIDRSRRKTNPLQFKEDGSIRRYKKGERPKWIISNHCKDYYVKLKELYRKQSVYRKNSHIELSNMLLNLGNDFIVENNPVEAWMRKSKEITFNKKGKINSRKRFGKSIANHAPSMFITILENKVKSLGGSLTKADIKNAASQFDFTNETFTKHELKERRINLSNGNTHCRDSIAAFNLQHLIGDKEYDVSTMKEKYNKFCRMEQEEIKRHQMFDEKISSNFGIN

SEQ ID NO: 303 - protein, 523 aa, unidentified
  REGION 1..523: note = MG91C-12 Effector
  REGION 1..523: note = uncultivated organism
MKSEKKNRCMEKEMFVVELPLKVEKWQSDVLNKRYELIAKIYNYAQKKLLRQYIYLVQMDEYKKCKTKKEKSIFFKNHPFYFKDITSKITFSEYDVTAFVEKIIKKNVSSDKTYKDFGINTSITECIGKHIWSAWEKKIYGNGKKVSLKNKGELNTISIRKKSEYFGGLDIDIEHMILSFNINGKTKSKAKKIILPINVKNLTEYEMYALKDGYDSINVVTIIRKMIRGNYKYYVQFTIEGEKPQKGRTLGNGNVGIDIGPSTVAISSLNGVTIDTLAEKCDNIESKISNIERKMDRSRRATNPQNFTEDGQIKRIDRKKGEKRVWKYSKRYVKLRNKKRELLRKQTVIRKLQHNIKANEILSLGDTFIVENNPISAWAKRANGVRKNKNGKIQTNKRFGKSIGNHAPAMFVTILQNKVESLKGKFVQVDLKNAASRFDFTNGDFTEHNLDERKITLSNGNTHQRDMLAAFNLQHIDLTNDDLKKYNVDEMIEDYPIFCKLEKEEIKRFIDKMKISNKSNIGL

SEQ ID NO: 304 - protein, 509 aa, unidentified
  REGION 1..509: note = MG91C-13 Effector; note = uncultivated organism
MNKRIKEEGKENFILCLPLQVTKQQADILNKRLSICCGIYNNFQKKMLRKFNYVKQMKAYKECKTLSERRKFLNTYTIQWKNTKGKLYDLKPFTEFGFKSYIAVFNETFSNNGINSKLLQYIASNAWSAWDKVLYGKGKRISFKKKDEFNSYTIGISSNNTFNGIDISKINENKIGVNLNGGRGKNAKVIYIPYKINTDYERECFLNDIRAIGLKRVFIRGKWKYFITFSFEGKKPVKNRKLGKGKVGIDIGPSTIAISSDDVVYMDVLAKNVNSIEDKKRLLQRKIDRSKRKTNPLQFKEDGSIRRYKKGERPKWIISNHCKDYYVKLKELYRKQSVYRKNSHIELSNMLLNLGNDFIVENNPVEAWMRKSKEITFNKKGKINSRKRFGKSIANHAPSMFITILENKVKSLGGSLTKADIKNAASQFDFTNETFTKHELKERRINLSNGNTHCRDSIAAFNLQHLIGDKEYDVSTMKEKYNKFCRMEQEEIKRHQMFDEKISSNFGIN

SEQ ID NO: 305 - protein, 521 aa, unidentified; identical to SEQ ID NO: 166
  REGION 1..521: note = MG91C-14 Effector; note = uncultivated organism

SEQ ID NO: 306 - protein, 605 aa, unidentified
  REGION 1..605: note = uncultivated organism; note = MG91C-15 Effector
MIEEFVLTLPLKVESWQADILNNRYEHLRRIYNMVQRKLLRHYIYLSQQNEYRQCKDNDEKRTFWENHPITFKDFCDKNGNPAIVKFPYTYSHRSKDSGKKASNGISDFVPKFKNRNIGGGLTYGSLGINASILENLGLRIEQAWKKRLYDPKSKHISFKEEGDLNTLGCREKNGFSGFVLDLQKMTLSFNTNGRQGKSAKFIHFPINAGKELTEYEMYALKGGIGSIRKIVVSREYVRGRYKYYVQLTIKGEKPQKNRTLGKGTVGIDVGPSTVAVSSLHGVSLDKLADKCDNIEHDLWRINRKMDRSKHETNPDFFNEDKTYKKPEKGERRKWTFSNRYKRLKLERRELYRKQAAISKQQHIDMANALLSQGDEFIVENNPIDSWAKRAKETKRGKNGKIRCKRRFGKSVAHHAPAMFVSILQNKVRSLGGTFHKVEIKNAATQFDFTNGEFTKHELNERTVTLSNGRTHQRDMLSAFNLQHLRPDSEETKDYNIEQMEADYPVFCKLEEKGIAQFKEKRQQERDVAYFTGVSHKTLKFSSANRHNGGVGRKNCNHHAGSKRNVAETGNKSSSKEYCESMETVSLVPNEYHHSNHQHLRLDGL

SEQ ID NO: 307 - protein, 461 aa, unidentified
  REGION 1..461: note = uncultivated organism; note = MG91C-16 Effector
MEKEMFVVELPLKVEKWQSDVLNKRYELLAKIYNYAQKKLLRQYIYLTQMDEYKKCKTKKDKSVFFKNHPFYFKGITSKITFSEYDVTAFVEKIIKKNVSCDKTYKDFGINSSITEYLGKHIWSAWEKKIYDNGKKVSFKNKGELNTISIRKKSEYFGGLDADIEHMILSFNINGKKNSKAKKIILPINVKNLTEYEMYALKDGYDSINVVTIIRKMIRGKYKYYVQFTIKGEKPQKGRTLGKGNVGIDIGPSTVAISSLNGVTIDTLAEKCNNIEDKISDIERKMDRSRRATNPQNFTEDGQIKRINRKKGEIREWKYSKRYVKLRSKKQELLRKQAAIRKLQHNIKANEILSLGDTFIVENNPISAWAKRAKGVRKNKNGKIQTNKRFGKSIGNRAPSMFVTILQNKVKSLKGKFVPVNPKNAASRFDFTNGDFTEHNLDERKITLSNGNTHQRDMLAA

SEQ ID NO: 308 - protein, 518 aa, unidentified
  REGION 1..518: note = MG91C-17 Effector; note = uncultivated organism
MNNKKEDDIFVLELPLSVEKWQSDLLDRRYEYLRQIYNYAQRKLIRQYIYLVQTEEYKKCRTKKDKSSFFGNHEFWFNGIKCPITFTEFGIKKYVTKLCKLKVSSGMTYKDFGLNSSHLETLGANMWAAWDKKIYGKGERINIKRYGCLNTFSVRCKSGYFGGLKVILDGNEIYLFLDMNGKKGEKEKKEIKLTVKIGKMTEYEMQALRNGIENIRMVTIARKLVRGKYKYYVQFAIKDEKPKKGRKLGKGSVGIDVGPSTVAVSSLHGVEINTLAEKCDDIHRKVRLIQRKMDRSSRASNPQNYNEDGTIKKVRGQKLKWVRSKNYVRLRNEKRELMRKQAAIRKLSHNIDTNKLLELGDTFVVEDNPISSWTKRASGIRKGKNGKNLSNKRYGKSVANHAPAAYVEILKNKVESLGGKFIKVDTKNGASGYDFTTGEYTKHGVEKRKVTLSNGNVHQRDMLAAFNLQHLNYEDGELKSYNVEEMEKDYPIFCKLEKEEIKRYTSGQKTSHKSTVGY

SEQ ID NO: 309 - protein, 630 aa, unidentified
  REGION 1..630: note = uncultivated organism; note = MG91C-18 Effector
MILVHFSLHHEICEFKFYFVNLQEIFSTPMIEEFVLTLPLKVELWQADILNKRYEHLRRIYNMVQRKLLRNYIYLSQQDAYRQCKDNNEKRDFWENHPVTIKEFCDKNGNPAIVKFPYTLSRRSKNSGKKASNGISDFVPKFKNHNIGSGLTYGALGINASILEELGLRIQQAWEKRLYNPKSKHISFKEEGDINTFGCREKRGFSGFVLDLSHMMLLVNINGRQHQLAQYLNLPINPNKELTEYEMYALKGGLSSVRKIIISREYIRGRYKYYVQLTIKGEKPQKNRTLGKGTVGIDVGPSTVAVSSLQGVSLDKLADKCDDIERDLWRINRKMDRSKRATNPDFFNKDKTYSKRKKDERRKWTFSNRYKRLKLERRELYRKQAAIRKQQHIEMANALLSQGDEFIVENNPIDSWAKRAKETKKGKNGKNRCKRRFGKSIAHHAPAMFVSILQNKVRSLGGSFREVEIKNAATQFDFTNGEFTKHELNERTVTLSNGRTHQRDMLAAFNLQHLKEEVKDYDIEQMDVDYPLFCKLEEKGIAQFKENRHLDRDVAYFVGASHKTLKFSSANRHNGGVGRKNCNHHSGGRDVAKTGNKSSIMKNCESLEPVSVLPNEYRHSNHQHLKIDGL

SEQ ID NO: 310 - protein, 549 aa, unidentified
  REGION 1..549: note = MG91C-19 Effector; note = uncultivated organism
MLCNTIIMVLTGCALKNLATEGVLCNAITMALTSFTHDIQFFPYITRYKTLSNMEEIFILEMPLHVEQWQADLLNKRYEYLRKLYNYVQSKLLKQYEYFIQMNEYKECTGSCKTPLFEFFKTHSFHFRGINNSQGLPLPITFSKMGICAYVCQLAKKNIGDNTTYKDVGINTCVLKHMGEHIWAAWDKMIFNKKTNRISFKKYGEMNILGAIKSGGYTCGFDIDLENFILTFNLNGKQGKNGKYIHLPILPNVKHADYETEALKGGYNSIRVLTIVRKLIRGKYKYYLQITIKGKKPQKNRTLGKGNVGIDIGPSTIAISCDSKVDVSQLADKCENIQHKIAVINRKLDRSRRATNPDNFNENGTYKPINRKNGERRTWYYSNRYKKLKAIRQELQRKQAAIRKLQHTEYVNTLLPLGNTFIVEKNPYCGWAKRSEGIRYDKNGKICSNKRYGHSIGIHAPSTLITLLENKVKSLGGKMFKVDIRNAATGYDFTNGEYTKHDISDRKITLSNGDVHHRDIMAAFNLKHLNYDSKELKDYNIPEMYQDYD

SEQ ID NO: 311 - protein, 630 aa, unidentified
  REGION 1..630: note = MG91C-20 Effector; note = uncultivated organism
MILVHFSLHHEICEFKFYFVNLQEIFSTPMIEEFVLTLPLKVELWQADILNKRYEHLRRIYNMVQRKLLRNYIYLSQQDAYRQCKDNNEKRDFWENHPVTIKEFCDKNGNPAIVKFPYTLSRRSKNSGKKASNGISDFVPKFKNHNIGSGLTYGALGINASILEELGLRIQQAWEKRLYNPKSKHISFKEEGDINTFGCREKRGFSGFVLDLSHMMLLVNINGRQHQLAQYLNLPINPNKELTEYEMYALKGGLSSVRKIIISREYIRGRYKYYVQLTIKGEKPQKNRTLGKGTVGIDVGPSTVAVSSLQGVSLDKLADKCDDIERDLWRINRKMDRSKRATNPDFFNKDKTYSKRKKDERRKWTFSNRYKRLKLERRELYRKQAAIRKQQHIEMANALLSQGDEFIVENNPIDSWAKRAKETKKGKNGKNRCKRRFGKSIAHHAPAMFVSILQNKVRSLGGSFREVEIKNAATQFDFTNGEFTKHELNERTVTLSNGRTHQRDMLAAFNLQHLKEEVKDYDIEQMDVDYPLFCKLEEKGIAQFKENRHLDRDVAYFVGASHKTLKFSSANRHNGGVGRKNCNHHSGGRDVAKTGNKSSIMKNCESLEPVSVLPNEYHHSNHQHLKIDGL

SEQ ID NO: 312 - protein, 516 aa, unidentified
  REGION 1..516: note = MG91C-21 Effector; note = uncultivated organism
MEKKKEMFVVELPLKVEKWQSDVLNKRYELITKIYNYAQKKLLRQYIYLTQMDDYKKCKTKKDKSVFFKNHPFYFKGITSKITFSEYDVTAFFEKIIKKNVSSDKTYKDFGINTSITGYLGKHAWSAWEKKIYGTGKKISFKNKGELSTISIRKKSEYFGGLDVDIEHMILSFNINGKTKSKAKKIILPINTKNLTEYEMYALKGGYDSIKVVTIVSKMIRGKYKYYVQFTIEGEKPQKGRTLGKGNVGIDIGPSTVAISSLNGVTIDTLAEKCDNIEDKISDIERKLDRSRRATNPQNFTEDGQIKRIDRKKGEKRKWEYSKRYVKLNNKKRELLRKQAAIRKLQHNIKANEVLSLGDTFIVENNPTSAWAKRAKGVRKNKNGKIQTNKRFGKSIGNHAPAMFVTILQNKVESLKGKFVKVDIKNAASRFDFTNGDFTEHNLDERKITLSNGNTHQRDMLAAFNLQHIDLTNDDLKKYNVEEMMEDYPIFCKLEKEEIKRFTNGMKIPNKSNIGI

SEQ ID NO: 313 - protein, 510 aa, unidentified; identical to SEQ ID NO: 177
  REGION 1..510: note = uncultivated organism; note = MG91C-22 Effector

SEQ ID NO: 314 - protein, 466 aa, unidentified
  REGION 1..466: note = MG91C-23 Effector; note = uncultivated organism
QMDEYKKCKTKKDKSAFFKNHPFYFKGITSKITFSEYDVTAFVEKIIKKNVSSDKTYKDFGINTSITECLGKHIWCAWEKKIYGKGKKVSFKNKGELNTISIRKKSEYFGGLDVDIEHMILSFNINGRTKTKAKKIILPINVKNLTEYEMYALKGGYDSINVVTIIRKMIRGKYKYYVQFTIEGEKPQKGRTLGKGNVGIDIGPSTIAISSLNGVTIDTLAEKCDNIEGKISDVERKMDRSRRVTNPQNFTEDGQIRRIDRKKGEKRVWKYSKRYIKLRNKKRELLRKQTVTRKLQHNIKANEILSLGDTFIVENNPMSAWAKRAKGVRKNKNGKIQTNKRFGKSIGNHAPAMFVTILQNKVESLKGKFVKVDPKNAASRFDFTNGDFTEHNLDERKITLSNGNTHQRDMLAAFNLQHIDLTNDDLKKYNVDEMIEDYPIFCKLEKEEIKRFINGMKKSNKSNIGI

SEQ ID NO: 315 - protein, 607 aa, unidentified
  REGION 1..607: note = MG91C-24 Effector; note = uncultivated organism
MIEEFVLTLPLKVETWQADILNKRYEHLRCIYNMVQRKLLRHYIYLSQQNEYRQCKDNDEKRTFWENHPITFKDFCDKNGNPAIVKFPYTYSHRSKDSGKKASNGISDFVPKFKNRNIGGGLTYGSLGINASILENLGLRIEQAWKKRLYDPKSKHISFKEEGDLNTLGCREKDGFSGFVLDLQKMMLSFNTNGRRHGSAEYIHLPIKPSKLTKYEKFVIDALKDKLSSVRKIVVAREYVRGKYKYYVQLTIKGEKPQKNRTLGKGTVGIDVGPSTVAVSSLQGVSLDKLADKCDNIEHDLWHINRKMDRSKRDTNPDFFNEDKTYKKPEKGERRKWTFSNRYKRLKLERRELYRKQAAIRKQQHIEMANALLSQGDEFIVENNPIDSWAKRAKETKRGKNGKIRCKRRFGKSVAHHAPAMFVSILQNKVRSLGGSFHKVEIKNAATQFDFTNGEFTKHELNERTVTLSNGRTHQRDMLSAFNLQHLRPDSEETKNYNIEQMEADYPVFCKLEEKGIAQFKEKRQQERDVAYFTGVSHKTLKFSSANRHNGGVGRGNCNYHAGSKRNVAKTGNKSSSKENCESMETVSLVPNEYHHSNHQHLRLDGL

SEQ ID NO: 316 - protein, 589 aa, unidentified
  REGION 1..589: note = MG91C-25 Effector; note = uncultivated organism
MSKSYEKCLWTAFLLGSDALLLKYYDYLCTLKLCSPIMIEEFVLTLPLKVESWQADILNNRYEHLRHIYNMVQRKLLRHYIYLSQQNEYRQCKDNDEKRMFWETHPVIIKDFCDKKGNPTIVKFPHTYYTRSKASGKRTSNGIPDLLPKLKNRNIGSGLTYASLGINAAILENLGLRIEQAWKKRLYDLKSKHISFKEEGDLNTFGCREKDGFSGFVLDLQKMKLSFNTNGRQGKSAKFIHFPINVGKELTEYEMYALKGGIGSVRKIVVAREYVRGRYKYYVQLTIKGEKPQKNRTLGKGTVGIDVGPSTVAVSSLQGVSLDKLADKCDNIEHDLWRINRKMDRSKHETNPDFFNEDKTYKKPEKGERRKWTFSNRYKRLKLERRELYRKQAAIRKQQHIEMANTLLSQGDEFIVENNPIDSWAKRAKETKRGKNGKIRCKRRFGKSVAHHAPAMFVSILQNKVRSLRGTFHKVEIKNAATQFDFTNGEFTKHELNERTVTLSNGRTHQRDMLSAFNLQHLRPDSEETKDYNIEQMEADYPVFCKLEEKGIAQFKEKRQQERDVAYFTGVSHKTLKFSSANRHNGGVG

SEQ ID NO: 317 - protein, 510 aa, unidentified
  REGION 1..510: note = uncultivated organism; note = MG91C-26 Effector
MFVVELPLKVEKWQSDVLNKRYELLAKIYNYAQTKLLRQYIYLTQMDEYKKCKTKQDKSVFFKNHPFYFKGITSKITFSEYDVTNFVNKIIKKNVSSDKTYKDFGINTSITEYLGKHAWSAWKKKIYGNGKKVSFKNKDELNTISIRKKSEYFGGLDVDIEHMILSFNINGRTKTKAKKIILPINVKNLTEYEMYALKDGYDSINVVTIMRKMIRGNYKYYVQFTIEGEKPQKGRTLGKGNVGIDIGPSTVAISSLNGVTIDTLAEKCDNIEDKISDIERKMDRSRRATNPQNFTEDGQIKRIDRKKGEKREWKYSKRYVKLHNKKRELLRKQAAIRKLRHNIKANEILSLGDTFIVENNPTSAWAKRAKGVRKNKNGKIQTNKRFGKSIGNHAPAMFVTILQNKVESLKGKFVKVDPKNAASRFDFTNSDFTEHNLDERKITLSNGNTHQRDMLAAFNLQHIDLTNDDLKKYNVNEMIEDYPIFCKLEKEEIKRFINGIKISNKSNIGI

SEQ ID NO: 318 - protein, 402 aa, unidentified
  REGION 1..402: note = uncultivated organism; note = MG91C-27 Effector
SITECLGKHIWSVWEKKIYDNGKKVSFKNKGEVNTISIRKKNEYFGGLDVDIEHMILSFNINGKIKAKAKKIILPINVKNLTEYEMYALQGGYDSINVVTIIRKMIRGKYKYYVQFTIEGEKPQKGRTLGKGNVGIDIGPSTVAISSLNGVTIDTLAEKCDNIESKISDIGRKMDRSRRATNPQNFTEDGQIKRIDRKKGEKRVWKCSKRYIKLRNKKRELLRKQTVIRKLQHNIKANEILSLGDTFIVENNPISAWAKRAKGVRKNKNGKIQTNKRFGKSIGNHAPSMFVTILQNKVKSLKGKFVTVNPKNAASRFDFTNGDFTEHNLDERKITLSNGNTHQRDMLAAFNLQHIDLTNEDLKKYNVHEMIEDYPIFCKLEKEEIKRFINGMKMSNKSNIGI

SEQ ID NO: 319 - protein, 723 aa, unidentified
  REGION 1..723: note = uncultivated organism; note = MG126-3 Effector
MAEEKEMRSYNIRINPDADAIKVIERDMKLMEQLYGFIIYKIESHIQQLRDETEYGEYDSNYKEYCRLMAEAEQKKRQCEVLRDAKKRKNIEDPELDSQYKELSAEIIKLDKHRKEWCRKCCEFFKNNKKALCHFVFESRRKVIRLKSDMVTPTENSGFILHHGVNGELYYIYDKTYYMMVEDSIRKANKGDGYSALVCECGVVTKRVSLNEDKTFKDKVYDGVAVLTDDGEIIMPYEYLRIDKLNKNNAHSFSILFVGEYVYSLNKNPKTKKSQKTSMAYKDFGFTSTDFFENIGNRLTLSYADFPKLLSQKHRKNDFMTIMCGYQSKLMDIMCDESGGYFVRMVTHSYGRGKKKYVDIPVLVSHRNKNRIGTERDMDTKVLEKKLKLLETGKTNVIGISKDVIREKSRYYVNIFLEHTGSLGLEYNENSLNQELGVGSVGVDPNMHQIAVYSDSLKKSTIFSLETIRGGIFKQKIYDCYSKLNSLQTKMSRCMDENNSGAKDENGNFIKGKKLFKSKRYLAMKKEVGELHRRIAELKETANIFIAHELLSYGNVFYIEDPRSAEKKRRMQGVVYDVKGRVVKNRKFSRSIQKFSISGMIYKALDVIESRGGKVYWINNKSAATAYVPKGDNVSHGMGERVFNWLGYKVQRDMKSGYVLSGCKELEQTKETYLNKPIYKVVSYDDERLLNDVDVFAESVKEEMERCRDLNVEDKTIGVEKYF

SEQ ID NO: 320 - protein, 572 aa, unidentified
  REGION 1..572: note = uncultivated organism; note = MG126-3 Effector
MPANVYQYGSQLKTVLTGEAEKQIRLQARFWNDLVEIEHEFSQRYRDVMNNADEKISSLNEEVGRRREEIEALRGEIKERRKKARSGRVDDSEIKKKIAELLAENKHLIQELKDYRAEVKIMVKPRLHELESERRARVKELRQQYATEGLYWGNYNAVLTSYQTARSRSMQSGGELRFRRFDGTGRLTCQIQGGMSVEEAFSGANNLFQIDPVPETAWNIPSRGESRKLCRTKAKIRITSDEEKNPVWIEIPIVMHRPIPQNARIQRVSISTKKVADRIRWFLNVTVIEDEPEKDDFAIGQVVALDVGWRKKEDGSIRVAYWIDSNGDTGEVELDVSFIHTDQRIRTLQKKRDDNFNVAKEKLSKWVSARKEHEGVPEWFTERTDTLAQWRAQSRLAALILFWRDNRFTGDGEVFSFLESWRRQDRHLWIWQANLRDKMSGRRLQQYRVFAAKAAKQYDAVIFEEFDLRSIKKKKRPEQGVDSDSSLRNFMKIAGVSILRSEVRRAFEATGKLFVKEQTSGQGACPFCAETAEREPGVGMFVSCTGCGKVYDRDWAKAKDLLSRWFVATKKL

SEQ ID NO: 321 - protein, 552 aa, unidentified
  REGION 1..552: note = MG126-4 Effector; note = uncultivated organism
MFGNKSLPSRIFSYGANAPIEGLDLVNEQMKLAHRYRNSLVELEHSRRKRVNEALARLSPRLVEVEELLIVADQKVEDAADAIRKASGAARKKVRPPEMVAEMKQCKEVLKALRADRKSLRTLLFASKAWEQEQDTLNAWVTEQQKALRAASGLYWGTYLHVEQSMAGTKSGPPPRFYRYDGTGHLAVQLQKGLSPKEAQENDPRIRIEVVPEGVYRPGKNRPKKLGNAIVHFRVGSDKSGDPIFARIPFILHRPLPEDSRIKWVHLISDRVGTHSKWLVQFVLSKESWPKADRATSGHVGVDVGWRMKEDGSLRVAVWKGSDGQDGELTLPLNWLPAMGKTRHIRSVRDKNFDTIRPELVAWLKERQDLPEWMKEPLTTMHMWKAQARLASFMIRWRTERFPGDEQMFQKAEDWRKRDRHLYDYESNLRDQLLAQRSDLYRNFAAKLRRLYKTAALEDLDLRDFHVLPPVEDPMMDPALKEHVRDACLSSLLQYLKESMGGFIEVPAANTTKKCHSCGSIQEWDHKILRHTCTKCGKDWDQDYNAAINIGA

SEQ ID NO: 322 - protein, 612 aa, unidentified
  REGION 1..612: note = MG126-5 Effector; note = uncultivated organism
MTARIYEYGLLAPSQEGERVSDQIWRAHRYRNLLCEIERRRREAVAAVLAAHPTIEPLSAAVDLLATEAEAADLAIRAARKASRSRSDTAAGRSALRDLRVRLKRARAEMRDAKRALRDDAPIQAQIAAVNEVDHCARLKARAECGVHWGSYLLIENAAIQAAKTAIKERTMPQFSAWDGNGAVGVQIQGGISVAEVHAGTDTRLQIDAVPEAAWTGTRGDRRRLGRTTLRMRVGSEGRAPIWAVWPMVMHRPLPAGAVLKGARVIRRRHGWRSRGSHDTWHLQITVVLPDEYRTEPCGTGMVAVHPGWRQVAGGLRIGYVLGDDGHERELLLDDSVAPRIASADAIHGHRDDAIEALRPALSAWLAGRDLPEWLAEQSRYLPMWRATSRFARLEARWRTERFAGDDEGYAMLDSWRRRDAHLYAYEDGRRDRALGHRREVYRRLAAELARRYEVLVLSDTDYRQLARRPRTEDERAAWDAANGQRHLAAVSTLTEACRQAFRARGGRIVTVASLDLTRTCHVCGAIVSWDQAAYVYATCACGATWDQDANAAHNSLARGREQLIADLAAEAARAKDPVVVESRWAKVKRLGREKREAARAADDNSIDVQRD

SEQ ID NO: 323 - protein, 612 aa, unidentified
  REGION 1..612: note = MG126-6 Effector; note = uncultivated organism
MTARIYEYGLRAPTQEGARVSDQIWLAHRYRNLLCEIERRRREAVAAVLAAHPTIEPLSAAVDLLATEAEAADLAIRAARKTSRSRSDTVADRSALRDLRVRLKRARAEMRDAKRALRDDAPIQAQIAAVNEVDHCARLKARAECGVHWGSYLLIENAAIQAAKTAIKERTMPQFSAWDGNGAVGVQIQGGISVAEVHAGTDTRLQIDAVPEAAWTGTRGDRRRLGRTTLRMRVGSEGRAPIWAVWPMVMHRPLPAGAVLKGARVIRRRHGWRSRGSHDTWHLQITVVLPDEYRTEPCGTGMVAVHPGWRQVAGGLRIGYVLGDDGHERELLLDDSVAPRIASADAIHGHRDDAIEALRPALSAWLAGRDLPEWLAEQSRYLPMWRATSRFARLEARWRTERFAGDDEGYAMLDSWRRRDAHLYAYEDGRRDRALGHRREVYRRLAAELARRYEVLVLSDTDYRQLARRPRTEDERAAWDAANGQRHLAAVSTLTEACRQAFRARGGRIVTVASLDLTRTCHACGAIVSWDQAAYVYATCACGATWDQDANAAHNSLARGREQLIADLAAEAARAKDPVVVESRWAKVKRLGREKREAARAADDNSIDVQRD

SEQ ID NO: 324 - protein, 678 aa, unidentified
  REGION 1..678: note = MG126-7 Effector; note = uncultivated organism
VKRKTSETPTRIYSYGCLLPIAGAELLEDQLRLANRYYNKLIEIERRRREAVDKITLSWSDDLGALDTAAQQALRLEEQYAIQKKDYGDKAPESAAMREIMRVARAELLCQAYELILSRRSEQSVSNLEALDKRVKSINEMLAVARAEVKALRAGKEKKVKSVNTKAAAARVKQLIQDQKAAKTDLQRERERLRTPALRCAQRAIGDIAGAEIRSARAACGLYSATYLKTEEAAGQAAKTSYPGLPHFQRYDGSGKIAVQLVGGISVKELFSGEDTQIRIKPVPEDTFYRRRHHRRLASRTVVKIRAGSNTDRTPKWVDLPIILHRPLPDDAVIKWAWIFRRRIGCRHEYRLQLVFESNTFRTGVQKAGRGQATIAIDLGWRKLEDGEIRVAYWLDDQGNKGQFLVPRYKPRTGDAVVFGHEKPEDLRSIRDKLLDAEKTTLRTWLAATSLPDWLKERTRYLHTWRAPGKLGALLTTWKKQRFDGDEEIVRKLEAWSKQDRHLLDWESSQRDRAQNHRREEYRLIANKIARVYSKIIIEVFDLRTFAKAKQPEDGLPSDGRRQRSNRMIASPSELRNVIRQAAVKTGAEVIEESAEYTTLTCALCGFTEKWDAAPAIIHTCGRCGEEWDQDENACRNLLGASGKAITSDGEALENGKGADPRGKLRRSEFRQETVVAE

SEQ ID NO: 325 - protein, 585 aa, unidentified; identical to SEQ ID NO: 19
  REGION 1..585: note = MG126-8 Effector; note = uncultivated organism

SEQ ID NO: 326 - RNA, 163 nt, unidentified
  source 1..163: mol_type = other RNA; note = uncultivated organism; MG119-3_IG1
attcttttattatgcgacttcgaacttgcaaagttaggaaaagttttcgtaaggaataactgaaaaaagttattatagtagccttttactcaaataattttaagcatagcaaactgttgatttacaaaataaatttaattacttttacactaaagtatttttt

SEQ ID NO: 327 - RNA, 163 nt, unidentified; identical to SEQ ID NO: 326
  source 1..163: mol_type = other RNA; note = uncultivated organism; MG119-3_IG2

SEQ ID NO: 328 - RNA, 205 nt, unidentified
  source 1..205: mol_type = other RNA; note = uncultivated organism; MG119-3_IG3
gctacctttgccacatacagagttctttaaaattttagatatgatttttatattccctttatgggaagggtgtgttggaacgccttagtttgaggtttgaatataaaaagtgaaacaaatggagatttgtaacaccttcaaacactcaaaaaattcatttattttttgatataaatgttggatttcaacgacttttactacatat

SEQ ID NO: 329 - RNA, 137 nt, unidentified
  source 1..137: mol_type = other RNA; note = uncultivated organism; MG119-4_IG1
aacttttgttttatacattaatatgggcaaaaatactaaattttacgaaatacacttaactttcattcgtctctaattacaatagcagactatttttcctttgaaaataactacctttgtcaatctttataaaaata

SEQ ID NO: 330 - RNA, 162 nt, unidentified
  source 1..162: mol_type = other RNA; note = uncultivated organism; MG119-4_IG2
atttctcgaaatagaatttacttgtggaatccctcatgcggttgcatgggggattttttatgggcgattttgagaatttgtgaaattctgaaaattatttgagttggatttgagacttacaattataattacaatatttgcggtggtaaaattaaaaagagt

SEQ ID NO: 331 - RNA, 107 nt, unidentified
  source 1..107: mol_type = other RNA; note = uncultivated organism; MG119-4_IG3
aaaagaaattttctaattggggctgctggaagtccttaacctgagataggaaactatactcgggaagtacgtcaaaacattgaaattaataaacttagaaactaacg

SEQ ID NO: 332 - RNA, 202 nt, unidentified
  source 1..202: mol_type = other RNA; note = uncultivated organism; MG119-4_IG4
taacactctgatatcattaaatataagcctatttccttctcaaatcaattaatagggggctgatgatgattgagaggaaataggcctattatgtatttaaaagagaagtaatatagaggtgaatatagtgtattttacatttaaaggcctcacaataaatgattaaaaaatagattttgtaatcactacatttaagaagatt

SEQ ID NO: 333 - RNA, 108 nt, unidentified
  source 1..108: mol_type = other RNA; note = uncultivated organism; MG120-1_IG1
aaacaaatggttaggtcaagggtttaattaatatttagagaaaaatcatagaaacacaaccagcatctcaattgttatcaacaacccaattgtgtttcgttttttgct

SEQ ID NO: 334 - RNA, 206 nt, unidentified
  source 1..206: mol_type = other RNA; note = uncultivated organism; MG120-1_IG2
gaacttgaaataggcatcaaaccatttgttgaggttaatgcgagcggccagttggctttaaaaacaggctggcctctcgcaacctttaccgcaaaggacttacgacaaaaaatcttttgttttggaaagtgtttacaggttgggaaacatagcctctcgcaacttttatcctaaacattgataataattagacttataacaatgcc

SEQ ID NO: 335 - RNA, 219 nt, unidentified
  source 1..219: mol_type = other RNA; note = uncultivated organism; MG120-1_IG3
acgatcttcgtctttatgataacggaacccgccaatcgcacctttcgaaatttatgaataaaatcgggggcggacttgactgggctgactttttctgataaaattcagccaacaaatggcagggtcgagttttgaatctcaggtaccaattatagattgtgtctataaattggatagttttagacaaatggctagatcgagtttaggaggtttgtgttt

SEQ ID NO: 336 - RNA, 489 nt, unidentified
  source 1..489: mol_type = other RNA; note = uncultivated organism; MG119-1_IG1
attcatcgttgtcggacgatcaaagattgttcgaattttcggagaaaatatgccgcggtgggtttaatgaaacttaacttttataaccattcaacttaatcataatctaatcaaatgatcaaattgattaagaaaatgattagattgagtcggttagataacggaggtgaagaaagtgtggatcaaacgtgagacttatgaacatcttcggatctgcgaggaaatgaaaacgaagcttgaagatgaagtcaagcggttatccgaattgatttcttcttagaatccggattgcaaagtaggcccatggtgtaaagattgcgcacatattggaagagattcatctgtggtgcgttattatacccctagcattgagtttggttgtgcaaagacagtagctggagaagttatttactgcaaaaaacacatccacgatatgtgcccggaatttgaaaagtcctcttcagattcatatttttcgttattgtacaa

SEQ ID NO: 337 - RNA, 1259 nt, unidentified
  source 1..1259: mol_type = other RNA; note = uncultivated organism; MG119-1_IG2
ttacattacgattgcggtggcggaatagacgggctgcgattgattcgcagttcggtaaaaccagacgggaagccaaggtggactgaatggtaacagtagacgctagacgggtagcaagatggaaacgaccgtcccgtagaccgatggtctcgccaaaatccacggggttcttcttgtatgtgaggtgcaaatcctcacccgtaatttgatttaggtgattcgtatgagaaaattgatttggtatttaagatcttgcttttgtaggcatgattgggttgtagaagagcaaccattcagagaatactttgaagggacgcttactgagcaaaccactgtagtgtctatgagatgcacaaaatgtggttggcatcagtcatataagaagtttaagttttgatttcatgcggcagtagctcagatggtagaggacgccaacaacggtgatcggtcgccggttcgagtccggcctgccgaatcaacataatgaggtaaaactatggagaaatacggagaaatatatttaccaaaacatcatagagccaaaaaaaatggatatgtagatgagcatattgtaatcgcagaacaaaaaatagggagatagttaaaaaaggatgaggtggttcatcataaagacaaagataaatccaataataaccctgaaaatctgatggtttttacaacaatggctgcacatacggcatttcatagaggaggaaaattgataagccttcccgatgggacttatgattgcgaacggctcccaataagatattgtgaaatatgtgggaagcaacttaaaacaaaacgagcaaaaagatgtcaaaaatgcgaccatataagacaaagaaaatgcgattggccaacaaaagagtagctaataaaagatttgaatgactttaaaaccaatgttgctctcgcagaaaaatatggggtaagtgataaaaccattgcaaaatggaagaaaaaaatactatcacaatctgaatctgaaaaataagctataagatacattcataatctatattcccggtaactgtttccagttacctataatatgctatcgtagctcagctggcagagcaatcgttttgtaaacgataggtcgtgggttcgagtcccaccgatagctccatccgaaaaagggtcagtagtagggagtgcaaaccagtgaccctgagacccgcaaggggcaacacccaagcgtgtaaggcgtagtacgctgttaggtattcaatcaagatgagggagtgaggcaagtc

SEQ ID NO: 338 - RNA, 173 nt, unidentified
  source 1..173: mol_type = other RNA; note = uncultivated organism; MG119-1_IG3
gtaggttcgcaccaaatatagggcgacttggcgtccttaaaccgagaagtgcatcagaaggtttacatagatgtaacactcggtaaggtaaatccgtaaggataatccaccccggcgaaatgccggccactaaaatgcataaagcgcaagttaatattttgtacggttttgcc

SEQ ID NO: 339 - RNA, 728 nt, unidentified
  source 1..728: mol_type = other RNA; note = uncultivated organism; MG119-1_IG4
aaataggagagagcatctaaattttccaaaacagaaaaataactgcctccttagctcaatcggcagagcgccagactcataatctgtgaggttccgggttcgatcccccgagggggcaccagtgataccaaaggacggaacaggcagccgtcgcccaatgttaggagatgcgggagtgtcaccccgcttggtatctcaacaactgaatagcccagacgggcaaaacatttccgagtgatccgtcatcgctcggagacatcgaggtgtagcgcagtggcagcgctcctgctttgggagcaggatgccgcgggttcgaatcccgccacctcgaccaaacgccgccgtggtggaatggcagacacaagcgacttaaaatctcttgctctcagagcgtatgggttcgagtcccatcggcggcaccatttgcttagttggcggaatcggcagacgcaccgcactcaaaatgcggcggagaaatccatgagggttcaaatccctcactaagcaccagtaaccctgttcgctatgctcacgtttgcgcagtttggttcgcgaccgtacttcaatctctgtgagaagtcgaggcggcggccgacgctgttcttcggacgcagggtctgtaataggggtgtagccaagcggcaaggcaaaggactttgactccttgattcgccggttcgatcccggccacccctgccaatttcattttaaagttcaggagggttttt

SEQ ID NO: 340 - RNA, 1074 nt, unidentified
  source 1..1074: mol_type = other RNA; note = uncultivated organism; MG119-1_IG5
aaattctatcgctgccattcgaaaatctgaaacagtgcttcatctatgttcgcgtaccacatcgatgttaaaagtaaaaaaggcacgcaccaatcggctaaattggattttaattctaattcgacgcgaaaatcgaataagaatctataaaaaattgctgtaaagctaaaagatcagaagctgaaagctcaaaactcaatgctcaaagtttatttgatcaaagctcaaagatctaaaagtcaaagagtacagctttacaaaatccaaggtctcaggtttcgggcaagcactttgctatcgggttctcctcttggctgaatggtagcgatttgaatataacttcacgtacacaagaaaaggagaaatcctgtttttgattcacaactgcgtgaagccacgcggctttgccgcgtatatcatctaatccagctgcatgaggcggacagatgaatgtcaacaaacgtaggtgataaaatgtacggcgcattaacctcagaagaagaaattgaagctcgcatgttgtttcgaggagctaaaaatccgctaatgcagattgagatcatctgcgatctatactaggttgatctgaacacagtctgcaaagctctcaaattagagtgcgaccccgagcttctgaaaaagaatttaagggcaactcattatgatgaaaatccgacagtatatgcaaagattgtattaaactgggggacaatcagacaattcgctttacaaacggggatttgtgaacaatccgtccgtaaagcattgcgaagacgtaatatggcagactctaaaattgggaaaagaattttagaagaactgcaaaattaggaggatacaacccatgccaaaagcgccggtttgcagtcgctgcggtaagcaatctgcgaaaactcactttgaaaacaacgataaaagtagggttttatcatatgcggcgtaaaacccctgtcttcagacatggggagtatgtcaatgtgatattccaagacctttttgtgaggtgttccaagtaaagggttataaaaatctttatcgatatcttatcgatatggtagataagaggtaatatct

SEQ ID NO: 341 - RNA, 259 nt, unidentified
  source 1..259: mol_type = other RNA; note = uncultivated organism; MG119-2_IG1
ttcgtcttcttttttctatttattaaaaagattagaatttatgttataaattacattgaactgtttatactaatattgttgcaaagttgatttgcacaaagtatactatgcttagttacacatcattagatgtgagggcaattttatgtcctaatattgagggtattaggtaacaacactttgttttaatagaacagtgtctaataaccactcgataagaccatataaaaggtataaatagtgaatttgaggttttata

SEQ ID NO: 342 - RNA, 464 nt, unidentified
  source 1..464: mol_type = other RNA; note = uncultivated organism; MG119-2_IG2
tatacatattttataatatgtaagggcgattttacgtcctaatggtgaggatgttgacgtaaaaacattttgtttgaaatatatcaatgtgttaatgtcactcgctaaaaccatacaaatgtgtaaaataataatttgaggttttatacctacttaattttgtatggttataaaaccaggaatatcaccttttaagaagtcttcttgttttacacctactagaatttgcatggttgtaaaacgataaaaaaataataagtcaagttcaagcggttttacacctaccaaaatttgtatggttgtaaaactgaatatttaactctcgacttatatacaagttgtacacatactatataccgattaaaatacacaatttagaaataaaaatcatcaaaatatagataaaaagaggtcaaaaagacctctttttttattttagaatataaatttaagtcttcagtata

SEQ ID NO: 343 - RNA, 332 nt, unidentified
  source 1..332: mol_type = other RNA; note = uncultivated organism; MG119-5_IG1
aaaaatataataaaatattgtttacaaacaattaaaaataaattataataatattagattttgaaagagaggtgaaacgaaataaaatttcaaggggaggcttagatcggtgaatccgagtcctacgtgcgaatcgcatatggatgccccataacctgagatagtcagcgatgaaattgttgatacataaggatttacgtaaggtgcgaatctataaaaaatacgctgatgaaactcggtcaaggtattttccgaaaggaaaagtcctaactaatgttaggtgaaaaactcgatcacaaagcccgatcgagccacttacaactgaatacgga

SEQ ID NO: 344 - RNA, 169 nt, unidentified
  source 1..169: mol_type = other RNA; note = uncultivated organism; MG119-5_IG2
agaggtggtggcatattgtttggatttggtttttatagcaatgttgtaaaaggatttttacaactaattggattaatgtttgtttcatttattattggatttgttttaggtgcatggattttttgataaaagaatggtaattgatacataaattaggagggatattcaa

SEQ ID NO: 345 - RNA, 129 nt, unidentified
  source 1..129: mol_type = other RNA; note = uncultivated organism; MG119-5_IG3
taatccacctaaatatgatgaatattcaagtaatggtgtttatatttggggtactgaaaattctataaaaaaatacttagaaacttataaatgaaataaattaaaacaatattaataaggagcggttta

SEQ ID NO: 346 - RNA, 208 nt, unidentified
  source 1..208: mol_type = other RNA; note = uncultivated organism; MG90-3_IG1
ggcgatggaacctctgaggttgtgtggaagtgtgttggtctggttgcatgatctgtatctgtccaggctaccagagagttgtaagataaagctatctcttcctggtggatggtctaccgggtgcagtacctttggcgtggggttagagcacgtcactggtcagtctgaccccagccgcggggctaggtagcggcaaaggagccttacc

SEQ ID NO: 347 - RNA, 167 nt, unidentified
  source 1..167: mol_type = other RNA; note = uncultivated organism; MG90-3_IG2
ggaggttgcacggtacgaatcgtccctgctcagcctgcgaggaggcgtcgaagtcctcgacagcagctagcttgccgatatctgccgatgcgtccaaaatacctccaggctaagctatactggaggcattctggccgctcgtgatcgaaatttaaggagtagaggtt

SEQ ID NO: 348 - RNA, 206 nt, unidentified
  source 1..206: mol_type = other RNA; note = uncultivated organism; MG119-3_IG1_adapters
gaagtgccattccgcctgacctattcttttattatgcgacttcgaacttgcaaagttaggaaaagttttcgtaaggaataactgaaaaaagttattatagtagccttttactcaaataattttaagcatagcaaactgttgatttacaaaataaatttaattacttttacactaaagtattttttaggctaggtggaggctcagtg

SEQ ID NO: 349 - RNA, 248 nt, unidentified
  source 1..248: mol_type = other RNA; note = uncultivated organism; MG119-3_IG2_adapters
gaagtgccattccgcctgacctgctacctttgccacatacagagttctttaaaattttagatatgatttttatattccctttatgggaagggtgtgttggaacgccttagtttgaggtttgaatataaaaagtgaaacaaatggagatttgtaacaccttcaaacactcaaaaaattcatttattttttgatataaatgttggatttcaacgacttttactacatataggctaggtggaggctcagtg

SEQ ID NO: 350 - RNA, 180 nt, unidentified
  source 1..180: mol_type = other RNA; note = uncultivated organism; MG119-3_IG3_adapters
gaagtgccattccgcctgacctaacttttgttttatacattaatatgggcaaaaatactaaattttacgaaatacacttaactttcattcgtctctaattacaatagcagactatttttcctttgaaaataactacctttgtcaatctttataaaaataaggctaggtggaggctcagtg

SEQ ID NO: 351 - RNA, 205 nt, unidentified
  source 1..205: mol_type = other RNA; note = uncultivated organism; MG119-4_IG1_adapters
gaagtgccattccgcctgacctatttctcgaaatagaatttacttgtggaatccctcatgcggttgcatgggggattttttatgggcgattttgagaatttgtgaaattctgaaaattatttgagttggatttgagacttacaattataattacaatatttgcggtggtaaaattaaaaagagtaggctaggtggaggctcagtg

SEQ ID NO: 352 - RNA, 150 nt, unidentified
  source 1..150: mol_type = other RNA; note = uncultivated organism; MG119-4_IG2_adapters
gaagtgccattccgcctgacctaaaagaaattttctaattggggctgctggaagtccttaacctgagataggaaactatactcgggaagtacgtcaaaacattgaaattaataaacttagaaactaacgaggctaggtggaggctcagtg

SEQ ID NO: 353 - RNA, 307 nt, unidentified
  source 1..307: mol_type = other RNA; note = uncultivated organism; MG119-4_IG3_adapters
gaagtgccattccgcctgaccttataaagtcgtctgcgatgatgtgcgcatgggtgttaataatcgtgtctttttttacttattttagttataaggtaatatacttataagggttattattgctaacgcatgtggttgcttatcacatacgttagcaatcaatcattacatacgttagtaataatcaacgactaacgtatgtaataatctgcaagaaaactttgccatttcaaataaaattgtaattttgctattacagtccccattaataaaaaaggaggtcagtaggctaggtggaggctcagtg

SEQ ID NO: 354 - RNA, 245 nt, unidentified
  source 1..245: mol_type = other RNA; note = uncultivated organism; MG119-4_IG4_adapters
gaagtgccattccgcctgaccttaacactctgatatcattaaatataagcctatttccttctcaaatcaattaatagggggctgatgatgattgagaggaaataggcctattatgtatttaaaagagaagtaatatagaggtgaatatagtgtattttacatttaaaggcctcacaataaatgattaaaaaatagattttgtaatcactacatttaagaagattaggctaggtggaggctcagtg

SEQ ID NO: 355 - RNA, 151 nt, unidentified
  source 1..151: mol_type = other RNA; note = uncultivated organism; MG120-1_IG1_adapters
gaagtgccattccgcctgacctaaacaaatggttaggtcaagggtttaattaatatttagagaaaaatcatagaaacacaaccagcatctcaattgttatcaacaacccaattgtgtttcgttttttgctaggctaggtggaggctcagtg

SEQ ID NO: 356 - RNA, 249 nt, unidentified
  source 1..249: mol_type = other RNA; note = uncultivated organism; MG120-1_IG2_adapters
gaagtgccattccgcctgacctgaacttgaaataggcatcaaaccatttgttgaggttaatgcgagcggccagttggctttaaaaacaggctggcctctcgcaacctttaccgcaaaggacttacgacaaaaaatcttttgttttggaaagtgtttacaggttgggaaacatagcctctcgcaacttttatcctaaacattgataataattagacttataacaatgccaggctaggtggaggctcagtg

SEQ ID NO: 357 - RNA, 262 nt, unidentified
  source 1..262: mol_type = other RNA; note = uncultivated organism; MG120-1_IG3_adapters
gaagtgccattccgcctgacctacgatcttcgtctttatgataacggaacccgccaatcgcacctttcgaaatttatgaataaaatcgggggcggacttgactgggctgactttttctgataaaattcagccaacaaatggcagggtcgagttttgaatctcaggtaccaattatagattgtgtctataaattggatagttttagacaaatggctagatcgagtttaggaggtttgtgtttaggctaggtggaggctcagtg

SEQ ID NO: 358 - RNA, 532 nt, unidentified
  source 1..532: mol_type = other RNA; note = uncultivated organism; MG119-1_IG1_adapters
gaagtgccattccgcctgacctattcatcgttgtcggacgatcaaagattgttcgaattttcggagaaaatatgccgcggtgggtttaatgaaacttaacttttataaccattcaacttaatcataatctaatcaaatgatcaaattgattaagaaaatgattagattgagtcggttagataacggaggtgaagaaagtgtggatcaaacgtgagacttatgaacatcttcggatctgcgaggaaatgaaaacgaagcttgaagatgaagtcaagcggttatccgaattgatttcttcttagaatccggattgcaaagtaggcccatggtgtaaagattgcgcacatattggaagagattcatctgtggtgcgttattatacccctagcattgagtttggttgtgcaaagacagtagctggagaagttatttactgcaaaaaacacatccacgatatgtgcccggaatttgaaaagtcctcttcagattcatatttttcgttattgtacaaaggctaggtggaggctcagtg

SEQ ID NO: 359 - RNA, 1302 nt, unidentified
  source 1..1302: mol_type = other RNA; note = uncultivated organism; MG119-1_IG2_adapters
gaagtgccattccgcctgacctttacattacgattgcggtggcggaatagacgggctgcgattgattcgcagttcggtaaaaccagacgggaagccaaggtggactgaatggtaacagtagacgctagacgggtagcaagatggaaacgaccgtcccgtagaccgatggtctcgccaaaatccacggggttcttcttgtatgtgaggtgcaaatcctcacccgtaatttgatttaggtgattcgtatgagaaaattgatttggtatttaagatcttgcttttgtaggcatgattgggttgtagaagagcaaccattcagagaatactttgaagggacgcttactgagcaaaccactgtagtgtctatgagatgcacaaaatgtggttggcatcagtcatataagaagtttaagttttgatttcatgcggcagtagctcagatggtagaggacgccaacaacggtgatcggtcgccggttcgagtccggcctgccgaatcaacataatgaggtaaaactatggagaaatacggagaaatatatttaccaaaacatcatagagccaaaaaaaatggatatgtagatgagcatattgtaatcgcagaacaaaaaatagggagatagttaaaaaaggatgaggtggttcatcataaagacaaagataaatccaataataaccctgaaaatctgatggtttttacaacaatggctgcacatacggcatttcatagaggaggaaaattgataagccttcccgatgggacttatgattgcgaacggctcccaataagatattgtgaaatatgtgggaagcaacttaaaacaaaacgagcaaaaagatgtcaaaaatgcgaccatataagacaaagaaaatgcgattggccaacaaaagagtagctaataaaagatttgaatgactttaaaaccaatgttgctctcgcagaaaaatatggggtaagtgataaaaccattgcaaaatggaagaaaaaaatactatcacaatctgaatctgaaaaataagctataagatacattcataatctatattcccggtaactgtttccagttacctataatatgctatcgtagctcagctggcagagcaatcgttttgtaaacgataggtcgtgggttcgagtcccaccgatagctccatccgaaaaagggtcagtagtagggagtgcaaaccagtgaccctgagacccgcaaggggcaacacccaagcgtgtaaggcgtagtacgctgttaggtattcaatcaagatgagggagtgaggcaagtcaggctaggtggaggctcagtg

SEQ ID NO: 360 - RNA, 216 nt, unidentified
  source 1..216: mol_type = other RNA; note = uncultivated organism; MG119-1_IG3_adapters
gaagtgccattccgcctgacctgtaggttcgcaccaaatatagggcgacttggcgtccttaaaccgagaagtgcatcagaaggtttacatagatgtaacactcggtaaggtaaatccgtaaggataatccaccccggcgaaatgccggccactaaaatgcataaagcgcaagttaatattttgtacggttttgccaggctaggtggaggctcagtg

SEQ ID NO: 361 - RNA, 771 nt, unidentified
  source 1..771: mol_type = other RNA; note = uncultivated organism; MG119-1_IG4_adapters
gaagtgccattccgcctgacctaaataggagagagcatctaaattttccaaaacagaaaaataactgcctccttagctcaatcggcagagcgccagactcataatctgtgaggttccgggttcgatcccccgagggggcaccagtgataccaaaggacggaacaggcagccgtcgcccaatgttaggagatgcgggagtgtcaccccgcttggtatctcaacaactgaatagcccagacgggcaaaacatttccgagtgatccgtcatcgctcggagacatcgaggtgtagcgcagtggcagcgctcctgctttgggagcaggatgccgcgggttcgaatcccgccacctcgaccaaacgccgccgtggtggaatggcagacacaagcgacttaaaatctcttgctctcagagcgtatgggttcgagtcccatcggcggcaccatttgcttagttggcggaatcggcagacgcaccgcactcaaaatgcggcggagaaatccatgagggttcaaatccctcactaagcaccagtaaccctgttcgctatgctcacgtttgcgcagtttggttcgcgaccgtacttcaatctctgtgagaagtcgaggcggcggccgacgctgttcttcggacgcagggtctgtaataggggtgtagccaagcggcaaggcaaaggactttgactccttgattcgccggttcgatcccggccacccctgccaatttcattttaaagttcaggagggtttttaggctaggtggaggctcagtg

SEQ ID NO: 362 - RNA, 1117 nt, unidentified
  source 1..1117: mol_type = other RNA; note = uncultivated organism; MG119-1_IG5_adapters
gaagtgccattccgcctgacctaaattctatcgctgccattcgaaaatctgaaacagtgcttcatctatgttcgcgtaccacatcgatgttaaaagtaaaaaaggcacgcaccaatcggctaaattggattttaattctaattcgacgcgaaaatcgaataagaatctataaaaaattgctgtaaagctaaaagatcagaagctgaaagctcaaaactcaatgctcaaagtttatttgatcaaagctcaaagatctaaaagtcaaagagtacagctttacaaaatccaaggtctcaggtttcgggcaagcactttgctatcgggttctcctcttggctgaatggtagcgatttgaatataacttcacgtacacaagaaaaggagaaatcctgtttttgattcacaactgcgtgaagccacgcggctttgccgcgtatatcatctaatccagctgcatgaggcggacagatgaatgtcaacaaacgtaggtgataaaatgtacggcgcattaacctcagaagaagaaattgaagctcgcatgttgtttcgaggagctaaaaatccgctaatgcagattgagatcatctgcgatctatactaggttgatctgaacacagtctgcaaagctctcaaattagagtgcgaccccgagcttctgaaaaagaatttaagggcaactcattatgatgaaaatccgacagtatatgcaaagattgtattaaactgggggacaatcagacaattcgctttacaaacggggatttgtgaacaatccgtccgtaaagcattgcgaagacgtaatatggcagactctaaaattgggaaaagaattttagaagaactgcaaaattaggaggatacaacccatgccaaaagcgccggtttgcagtcgctgcggtaagcaatctgcgaaaactcactttgaaaacaacgataaaagtagggttttatcatatgcggcgtaaaacccctgtcttcagacatggggagtatgtcaatgtgatattccaagacctttttgtgaggtgttccaagtaaagggttataaaaatctttatcgatatcttatcgatatggtagataagaggtaatatctaggctaggtggaggctcagtg

SEQ ID NO: 363 - RNA, 302 nt, unidentified
  source 1..302: mol_type = other RNA; note = uncultivated organism; MG119-2_IG1_adapters
gaagtgccattccgcctgacctttcgtcttcttttttctatttattaaaaagattagaatttatgttataaattacattgaactgtttatactaatattgttgcaaagttgatttgcacaaagtatactatgcttagttacacatcattagatgtgagggcaattttatgtcctaatattgagggtattaggtaacaacactttgttttaatagaacagtgtctaataaccactcgataagaccatataaaaggtataaatagtgaatttgaggttttataaggctaggtggaggctcagtg

SEQ ID NO: 364 - RNA, 507 nt, unidentified
  source 1..507: mol_type = other RNA; note = uncultivated organism; MG119-2_IG2_adapters
gaagtgccattccgcctgaccttatacatattttataatatgtaagggcgattttacgtcctaatggtgaggatgttgacgtaaaaacattttgtttgaaatatatcaatgtgttaatgtcactcgctaaaaccatacaaatgtgtaaaataataatttgaggttttatacctacttaattttgtatggttataaaaccaggaatatcaccttttaagaagtcttcttgttttacacctactagaatttgcatggttgtaaaacgataaaaaaataataagtcaagttcaagcggttttacacctaccaaaatttgtatggttgtaaaactgaatatttaactctcgacttatatacaagttgtacacatactatataccgattaaaatacacaatttagaaataaaaatcatcaaaatatagataaaaagaggtcaaaaagacctctttttttattttagaatataaatttaagtcttcagtataaggctaggtggaggctcagtg

SEQ ID NO: 365 - RNA, 375 nt, unidentified
  source 1..375: mol_type = other RNA; note = uncultivated organism; MG119-5_IG1_adapters
gaagtgccattccgcctgacctaaaaatataataaaatattgtttacaaacaattaaaaataaattataataatattagattttgaaagagaggtgaaacgaaataaaatttcaaggggaggcttagatcggtgaatccgagtcctacgtgcgaatcgcatatggatgccccataacctgagatagtcagcgatgaaattgttgatacataaggatttacgtaaggtgcgaatctataaaaaatacgctgatgaaactcggtcaaggtattttccgaaaggaaaagtcctaactaatgttaggtgaaaaactcgatcacaaagcccgatcgagccacttacaactgaatacggaaggctaggtggaggctcagtg

SEQ ID NO: 366 - RNA, 212 nt, unidentified
  source 1..212: mol_type = other RNA; note = uncultivated organism; MG119-5_IG2_adapters
gaagtgccattccgcctgacctagaggtggtggcatattgtttggatttggtttttatagcaatgttgtaaaaggatttttacaactaattggattaatgtttgtttcatttattattggatttgttttaggtgcatggattttttgataaaagaatggtaattgatacataaattaggagggatattcaaaggctaggtggaggctcagtg

SEQ ID NO: 367 - RNA, 172 nt, unidentified
  source 1..172: mol_type = other RNA; note = uncultivated organism; MG119-5_IG3_adapters
gaagtgccattccgcctgaccttaatccacctaaatatgatgaatattcaagtaatggtgtttatatttggggtactgaaaattctataaaaaaatacttagaaacttataaatgaaataaattaaaacaatattaataaggagcggtttaaggctaggtggaggctcagtg

SEQ ID NO: 368 - RNA, 251 nt, unidentified
  source 1..251: mol_type = other RNA; note = uncultivated organism; MG90-3_IG1_adapters
gaagtgccattccgcctgacctggcgatggaacctctgaggttgtgtggaagtgtgttggtctggttgcatgatctgtatctgtccaggctaccagagagttgtaagataaagctatctcttcctggtggatggtctaccgggtgcagtacctttggcgtggggttagagcacgtcactggtcagtctgaccccagccgcggggctaggtagcggcaaaggagccttaccaggctaggtggaggctcagtg

SEQ ID NO: 369 - RNA, 210 nt, unidentified
  source 1..210: mol_type = other RNA; note = uncultivated organism; MG90-3_IG2_adapters
gaagtgccattccgcctgacctggaggttgcacggtacgaatcgtccctgctcagcctgcgaggaggcgtcgaagtcctcgacagcagctagcttgccgatatctgccgatgcgtccaaaatacctccaggctaagctatactggaggcattctggccgctcgtgatcgaaatttaaggagtagaggttaggctaggtggaggctcagtg

SEQ ID NO: 370 - DNA, 121 bp, unidentified
  source 1..121: mol_type = other DNA; note = uncultivated organism; 119-3_5U40_31_F
taatacgactcactataggttgtaaaagcattaatttttaaatgaatatccaactggagatatcttgaaccttgcatccccggaagttgtaaaagcattaatttttaaatgaatatccaac

SEQ ID NO: 371 - DNA, 121 bp, unidentified
  source 1..121: mol_type = other DNA; note = uncultivated organism; 119-3_5U40_31_R
taatacgactcactataggttggatattcatttaaaaattaatgcttttacaactggagatatcttgaaccttgcatccccggaagttggatattcatttaaaaattaatgcttttacaac

SEQ ID NO: 372 - DNA, 121 bp, unidentified
  source 1..121: mol_type = other DNA; note = uncultivated organism; 119-4_5U40_31_F
taatacgactcactataggttggttttatcattgttttgaatggtatatccaactggagatatcttgaaccttgcatccccggaagttggttttatcattgttttgaatggtatatccaac

SEQ ID NO: 373 - DNA, 121 bp, unidentified
  source 1..121: mol_type = other DNA; note = uncultivated organism; 119-4_5U40_31_R
taatacgactcactataggttggatataccattcaaaacaatgataaaaccaactggagatatcttgaaccttgcatccccggaagttggatataccattcaaaacaatgataaaaccaac

SEQ ID NO: 374 - DNA, 127 bp, unidentified
  source 1..127: mol_type = other DNA; note = uncultivated organism; 120-1_5U40_37_F
taatacgactcactatagatagcaacgaatgaaaatacaaatggttagatcgagtggagatatcttgaaccttgcatccccggaagagagtatagcaacgaatgaaaatacaaatggttagatcgag

SEQ ID NO: 375 - DNA, 127 bp, unidentified
  source 1..127: mol_type = other DNA; note = uncultivated organism; 120-1_5U40_37_R
taatacgactcactatagctcgatctaaccatttgtattttcattcgttgctattggagatatcttgaaccttgcatccccggaagagagtctcgatctaaccatttgtattttcattcgttgctat

SEQ ID NO: 376 - DNA, 128 bp, unidentified
  source 1..128: mol_type = other DNA; note = uncultivated organism; 118-1_5U40_38_R
taatacgactcactataggtaacgactgataaaaattgctcactacggtgagactggagatatcttgaaccttgcatccccggaagagagtcgtaacgactgataaaaattgctcactacggtgagac

SEQ ID NO: 377 - DNA, 114 bp, unidentified
  source 1..114: mol_type = other DNA; note = uncultivated organism; 119-1_5U67_32_F
taatacgactcactataggacgcaacttgcgcgtatgcacaagtggaagtgtcgaggcttgcgacgtggtggctcacggaaggacgcaacttgcgcgtatgcacaagtggaagt

SEQ ID NO: 378 - DNA, 114 bp, unidentified
  source 1..114: mol_type = other DNA; note = uncultivated organism; 119-1_5U67_32_R
taatacgactcactatagacttccacttgtgcatacgcgcaagttgcgtcgtcgaggcttgcgacgtggtggctcacggaagacttccacttgtgcatacgcgcaagttgcgtc

SEQ ID NO: 379 - DNA, 102 bp, unidentified
  source 1..102: mol_type = other DNA; note = uncultivated organism; 119-2_5U40_28_F
taatacgactcactatagcctacttaattttgtatggttataaaactggagatatcttgaaccttgcatccccgcctacttaattttgtatggttataaaac

SEQ ID NO: 380 - DNA, 102 bp, unidentified
  source 1..102: mol_type = other DNA; note = uncultivated organism; 119-2_5U40_28_R
taatacgactcactataggttttataaccatacaaaattaagtaggtggagatatcttgaaccttgcatccccggttttataaccatacaaaattaagtagg

SEQ ID NO: 381 - DNA, 111 bp, unidentified
  source 1..111: mol_type = other DNA; note = uncultivated organism; 119-5_5U40_31_F
taatacgactcactataggttgcaatacgtatgcgtatgtaagtggtaatggagatatcttgaaccttgcatccccggaagttgcaatacgtatgcgtatgtaagtggtaa

SEQ ID NO: 382 - DNA, 111 bp, unidentified
  source 1..111: mol_type = other DNA; note = uncultivated organism; 119-5_5U40_31_R
taatacgactcactatagttaccacttacatacgcatacgtattgcaactggagatatcttgaaccttgcatccccggaattaccacttacatacgcatacgtattgcaac

SEQ ID NO: 383 - DNA, 129 bp, unidentified
  source 1..129: mol_type = other DNA; note = uncultivated organism; 90-3_5U67_37_F
taatacgactcactatagggtgcagtacctttggcgtggggttagagcacgtcacgtcgaggcttgcgacgtggtggctcacggaagacgaaggtgcagtacctttggcgtggggttagagcacgtcac

SEQ ID NO: 384 - DNA, 129 bp, unidentified
  source 1..129: mol_type = other DNA; note = uncultivated organism; 90-3_5U67_37_R
taatacgactcactataggtgacgtgctctaaccccacgccaaaggtactgcaccgtcgaggcttgcgacgtggtggctcacggaagacgaagtgacgtgctctaaccccacgccaaaggtactgcacc

SEQ ID NO: 385 - DNA, 164 bp, unidentified
  source 1..164: mol_type = other DNA; note = uncultivated organism; 119-3_5U40_31_F_adapters
gaagtgccattccgcctgaccttaatacgactcactataggttgtaaaagcattaatttttaaatgaatatccaactggagatatcttgaaccttgcatccccggaagttgtaaaagcattaatttttaaatgaatatccaacaggctaggtggaggctcagtg

SEQ ID NO: 386 - DNA, 164 bp, unidentified
  source 1..164: mol_type = other DNA; note = uncultivated organism; 119-3_5U40_31_R_adapters
gaagtgccattccgcctgaccttaatacgactcactataggttggatattcatttaaaaattaatgcttttacaactggagatatcttgaaccttgcatccccggaagttggatattcatttaaaaattaatgcttttacaacaggctaggtggaggctcagtg

SEQ ID NO: 387 - DNA, 164 bp, unidentified
  source 1..164: mol_type = other DNA; note = uncultivated organism; 119-4_5U40_31_F_adapters
gaagtgccattccgcctgaccttaatacgactcactataggttggttttatcattgttttgaatggtatatccaactggagatatcttgaaccttgcatccccggaagttggttttatcattgttttgaatggtatatccaacaggctaggtggaggctcagtg

SEQ ID NO: 388 - DNA, 164 bp, unidentified
  source 1..164: mol_type = other DNA; note = uncultivated organism; 119-4_5U40_31_R_adapters
gaagtgccattccgcctgaccttaatacgactcactataggttggatataccattcaaaacaatgataaaaccaactggagatatcttgaaccttgcatccccggaagttggatataccattcaaaacaatgataaaaccaacaggctaggtggaggctcagtg

SEQ ID NO: 389 - DNA, 170 bp, unidentified
  source 1..170: mol_type = other DNA; note = uncultivated organism; 120-1_5U40_37_F_adapters
gaagtgccattccgcctgaccttaatacgactcactatagatagcaacgaatgaaaatacaaatggttagatcgagtggagatatcttgaaccttgcatccccggaagagagtatagcaacgaatgaaaatacaaatggttagatcgagaggctaggtggaggctcagtg

SEQ ID NO: 390 - DNA, 170 bp, unidentified
  source 1..170: mol_type = other DNA; note = uncultivated organism; 120-1_5U40_37_R_adapters
gaagtgccattccgcctgaccttaatacgactcactatagctcgatctaaccatttgtattttcattcgttgctattggagatatcttgaaccttgcatccccggaagagagtctcgatctaaccatttgtattttcattcgttgctataggctaggtggaggctcagtg

SEQ ID NO: 391 - DNA, 171 bp, unidentified
  source 1..171: mol_type = other DNA; note = uncultivated organism; 118-1_5U40_38_R_adapters
gaagtgccattccgcctgaccttaatacgactcactataggtaacgactgataaaaattgctcactacggtgagactggagatatcttgaaccttgcatccccggaagagagtcgtaacgactgataaaaattgctcactacggtgagacaggctaggtggaggctcagtg

SEQ ID NO: 392 - DNA, 157 bp, unidentified
  source 1..157: mol_type = other DNA; note = uncultivated organism; 119-1_5U67_32_F_adapters
gaagtgccattccgcctgaccttaatacgactcactataggacgcaacttgcgcgtatgcacaagtggaagtgtcgaggcttgcgacgtggtggctcacggaaggacgcaacttgcgcgtatgcacaagtggaagtaggctaggtggaggctcagtg

SEQ ID NO: 393 - DNA, 157 bp, unidentified
  source 1..157: mol_type = other DNA; note = uncultivated organism; 119-1_5U67_32_R_adapters
gaagtgccattccgcctgaccttaatacgactcactatagacttccacttgtgcatacgcgcaagttgcgtcgtcgaggcttgcgacgtggtggctcacggaagacttccacttgtgcatacgcgcaagttgcgtcaggctaggtggaggctcagtg

SEQ ID NO: 394 - DNA, 145 bp, unidentified
  source 1..145: mol_type = other DNA; note = uncultivated organism; 119-2_5U40_28_F_adapters
gaagtgccattccgcctgaccttaatacgactcactatagcctacttaattttgtatggttataaaactggagatatcttgaaccttgcatccccgcctacttaattttgtatggttataaaacaggctaggtggaggctcagtg

SEQ ID NO: 395 - DNA, 145 bp, unidentified
  source 1..145: mol_type = other DNA; note = uncultivated organism; 119-2_5U40_28_R_adapters
gaagtgccattccgcctgaccttaatacgactcactataggttttataaccatacaaaattaagtaggtggagatatcttgaaccttgcatccccggttttataaccatacaaaattaagtaggaggctaggtggaggctcagtg

SEQ ID NO: 396 - DNA, 154 bp, unidentified
  source 1..154: mol_type = other DNA; note = uncultivated organism; 119-5_5U40_31_F_adapters
gaagtgccattccgcctgaccttaatacgactcactataggttgcaatacgtatgcgtatgtaagtggtaatggagatatcttgaaccttgcatccccggaagttgcaatacgtatgcgtatgtaagtggtaaaggctaggtggaggctcagtg

SEQ ID NO: 397 - DNA, 154 bp, unidentified
  source 1..154: mol_type = other DNA; note = uncultivated organism; 119-5_5U40_31_R_adapters
gaagtgccattccgcctgaccttaatacgactcactatagttaccacttacatacgcatacgtattgcaactggagatatcttgaaccttgcatccccggaattaccacttacatacgcatacgtattgcaacaggctaggtggaggctcagtg

SEQ ID NO: 398 - DNA, 172 bp, unidentified
  source 1..172: mol_type = other DNA; note = uncultivated organism; 90-3_5U67_37_F_adapters
gaagtgccattccgcctgaccttaatacgactcactatagggtgcagtacctttggcgtggggttagagcacgtcacgtcgaggcttgcgacgtggtggctcacggaagacgaaggtgcagtacctttggcgtggggttagagcacgtcacaggctaggtggaggctcagtg

SEQ ID NO: 399 - DNA, 172 bp, unidentified
  source 1..172: mol_type = other DNA; note = uncultivated organism; 90-3_5U67_37_R_adapters
gaagtgccattccgcctgaccttaatacgactcactataggtgacgtgctctaaccccacgccaaaggtactgcaccgtcgaggcttgcgacgtggtggctcacggaagacgaagtgacgtgctctaaccccacgccaaaggtactgcaccaggctaggtggaggctcagtg

SEQ ID NO: 400 - RNA, 61 nt, unidentified
  source 1..61: mol_type = other RNA; note = uncultivated organism; MG118-1_U40_18nt_target
taatacgactcactatagtaaaaattgctcactacggtgagactggagatatcttgaacct

SEQ ID NO: 401 - RNA, 61 nt, unidentified
  source 1..61: mol_type = other RNA; note = uncultivated organism; MG118-1_U67_18nt_target
taatacgactcactatagtaaaaattgctcactacggtgagacgtcgaggcttgcgacgtg

SEQ ID NO: 402 - RNA, 169 nt, unidentified
  source 1..169: mol_type = other RNA; note = uncultivated organism; 90-3_sgRNA_10bp_RAR_U40_24_target
taatacgactcactatagaggttgtgtggaagtgtgttggtctggttgcatgatctgtatctgtccaggctaccagagagttgtaagataaagctatctcttcctggtggatggtctaccgggtgcagtacgaaagtactgcacctggagatatcttgaaccttgcatc

SEQ ID NO: 403 - RNA, 181 nt, unidentified
  source 1..181: mol_type = other RNA; note = uncultivated organism; 90-3_sgRNA_16bp_RAR_U40_24_target
taatacgactcactatagaggttgtgtggaagtgtgttggtctggttgcatgatctgtatctgtccaggctaccagagagttgtaagataaagctatctcttcctggtggatggtctaccgggtgcagtacctttgggaaaccaaaggtactgcacctggagatatcttgaaccttgcatc

SEQ ID NO: 404 - RNA, 141 nt, unidentified
  source 1..141: mol_type = other RNA; note = uncultivated organism; 119-1_sgRNA_10bp_RAR_U40_24_target
taatacgactcactatagtttacatagatgtaacactcggtaaggtaaatccgtaaggataatccaccccggcgaaatgccggccactaaaatgcatgaaaatgcacaagtggaagttggagatatcttgaaccttgcatc

SEQ ID NO: 405 - RNA, 156 nt, unidentified
  source 1..156: mol_type = other RNA; note = uncultivated organism; 119-1_sgRNA_15bp_RAR_U40_24_target
taatacgactcactatagtttacatagatgtaacactcggtaaggtaaatccgtaaggataatccaccccggcgaaatgccggccactaaaatgcataaagcgcgaaattgcgcgtatgcacaagtggaagttggagatatcttgaaccttgcatc

SEQ ID NO: 406 - RNA, 160 nt, unidentified
  source 1..160: mol_type = other RNA; note = uncultivated organism; 119-2_sgRNA_10bp_RAR_U40_24_target
taatacgactcactatagtgacgtaaaaacattttgtttgaaatatatcaatgtgttaatgtcactcgctaaaaccatacaaatgtgtaaaataataatttgaggttttatacctacttaatgaaaattaagtaggtggagatatcttgaaccttgcatc

SEQ ID NO: 407 - RNA, 172 nt, unidentified
  source 1..172: mol_type = other RNA; note = uncultivated organism; 119-2_sgRNA_16bp_RAR_U40_24_target
taatacgactcactatagtgacgtaaaaacattttgtttgaaatatatcaatgtgttaatgtcactcgctaaaaccatacaaatgtgtaaaataataatttgaggttttatacctacttaattttgtagaaatacaaaattaagtaggtggagatatcttgaaccttgcatc

SEQ ID NO: 408 - RNA, 131 nt, unidentified
  source 1..131: mol_type = other RNA; note = uncultivated organism; 119-5_sgRNA_9bp_RAR_U40_24_target
taatacgactcactatagttttccgaaaggaaaagtcctaactaatgttaggtgaaaaactcgatcacaaagcccgatcgagccacttacagaaatgtaagtggtaatggagatatcttgaaccttgcatc

SEQ ID NO: 409 - RNA, 151 nt, unidentified
  source 1..151: mol_type = other RNA; note = uncultivated organism; 119-5_sgRNA_16bp_RAR_U40_24_target
taatacgactcactatagttttccgaaaggaaaagtcctaactaatgttaggtgaaaaactcgatcacaaagcccgatcgagccacttacaactgaatacggaaacgtatgcgtatgtaagtggtaatggagatatcttgaaccttgcatc

SEQ ID NO: 410 - RNA, 43 nt, unidentified
  source 1..43: mol_type = other RNA; note = uncultivated organism; MG118-1_U40_18nt
taaaaattgctcactacggtgagactggagatatcttgaacct

SEQ ID NO: 411 - RNA, 43 nt, unidentified
  source 1..43: mol_type = other RNA; note = uncultivated organism; MG118-1_U67_18nt
taaaaattgctcactacggtgagacgtcgaggcttgcgacgtg

SEQ ID NO: 412 - RNA, 151 nt, unidentified
  source 1..151: mol_type = other RNA; note = uncultivated organism, 90-3_sgRNA_10bp_RAR_U40_24
aggttgtgtggaagtgtgttggtctggttgcatgatctgtatctgtccaggctaccagagagttgtaagataaagctatctcttcctggtggatggtctaccgggtgcagtacgaaagtactgcacctggagatatcttgaaccttgcatc

SEQ ID NO: 413 - RNA, 163 nt, unidentified
  source 1..163: mol_type = other RNA; note = uncultivated organism; 90-3_sgRNA_16bp_RAR_U40_24
aggttgtgtggaagtgtgttggtctggttgcatgatctgtatctgtccaggctaccagagagttgtaagataaagctatctcttcctggtggatggtctaccgggtgcagtacctttgggaaaccaaaggtactgcacctggagatatcttgaaccttgcatc

SEQ ID NO: 414 - RNA, 123 nt, unidentified
  source 1..123: mol_type = other RNA; note = uncultivated organism; 119-1_sgRNA_10bp_RAR_U40_24
tttacatagatgtaacactcggtaaggtaaatccgtaaggataatccaccccggcgaaatgccggccactaaaatgcatgaaaatgcacaagtggaagttggagatatcttgaaccttgcatc

SEQ ID NO: 415 - RNA, 138 nt, unidentified
  source 1..138: mol_type = other RNA; note = uncultivated organism; 119-1_sgRNA_15bp_RAR_U40_24
tttacatagatgtaacactcggtaaggtaaatccgtaaggataatccaccccggcgaaatgccggccactaaaatgcataaagcgcgaaattgcgcgtatgcacaagtggaagttggagatatcttgaaccttgcatc

SEQ ID NO: 416 - RNA, 142 nt, unidentified
  source 1..142: mol_type = other RNA; note = uncultivated organism; 119-2_sgRNA_10bp_RAR_U40_24
tgacgtaaaaacattttgtttgaaatatatcaatgtgttaatgtcactcgctaaaaccatacaaatgtgtaaaataataatttgaggttttatacctacttaatgaaaattaagtaggtggagatatcttgaaccttgcatc

SEQ ID NO: 417 - RNA, 154 nt, unidentified
  source 1..154: mol_type = other RNA; note = uncultivated organism; 119-2_sgRNA_16bp_RAR_U40_24
tgacgtaaaaacattttgtttgaaatatatcaatgtgttaatgtcactcgctaaaaccatacaaatgtgtaaaataataatttgaggttttatacctacttaattttgtagaaatacaaaattaagtaggtggagatatcttgaaccttgcatc

SEQ ID NO: 418 - RNA, 113 nt, unidentified
  source 1..113: mol_type = other RNA; note = uncultivated organism; 119-5_sgRNA_9bp_RAR_U40_24
ttttccgaaaggaaaagtcctaactaatgttaggtgaaaaactcgatcacaaagcccgatcgagccacttacagaaatgtaagtggtaatggagatatcttgaaccttgcatc

SEQ ID NO: 419 - RNA, 133 nt, unidentified
  source 1..133: mol_type = other RNA; note = uncultivated organism; 119-5_sgRNA_16bp_RAR_U40_24
ttttccgaaaggaaaagtcctaactaatgttaggtgaaaaactcgatcacaaagcccgatcgagccacttacaactgaatacggaaacgtatgcgtatgtaagtggtaatggagatatcttgaaccttgcatc

SEQ ID NO: 420 - protein, 464 aa, unidentified
  source 1..464: note = uncultivated organism; MG119-124 Effector
MKTTRKLKLTIIGDEETRKEQYKIIREEQYQQYKALNLCMTLLNTHNILNSYNTGAENKLNAQIDSIDKKIEQAKKELEKKGLKESKVSKLKETIEFLENDREKLKDEYLNSSKFRSDIDEKMKEMYIKDMYTVVQNQVNFRARDMMSLVTQRARKDFKNSLKNGMAKGERSLTNYKRDFPLMTRGERWLKFEYDKDSDDILINWIHGIKFKVLLGYKKNENSIELRHTLHKVINKEYKICDSSMQFDRNNNLILNLTLDIPDKQNNNYIEKRTLGVDLGIKYPAYVCLNDDTYIRSHIGESLELLKQREQFKDRRKRLQQQLKNVKGGKGRNKKLSALNKLSDNERNFARTYNHMISKRIVEFAKKNRCEFINLEKLTKDGFDNNILSNWSYYELQNMIEYKAKREGIKVRYIDPAYTSQKCSRCGYIDKENRQTQEKFKCLKCEFEINADHNAAINIARALD

SEQ ID NO: 421 - protein, 403 aa, unidentified
  source 1..403: note = uncultivated organism; MG119-125 Effector
MSITVRKLKLIIVNSDDKARNNQYKFIRDSQYAQYQALNLGMGYMMAGYLACNRDIKSEEFKEWQKGLKNSNPVFQGIEYGKGIDTLSAVTQKIKKDFSTSLKNGLEKGERSSINYKRNFPLMTRGRNLNFYEEDGDIFIKWVNKILFKVVFNKRKENTIELQHTLNKVINGEYKVSQSSLEFDRNNNLILNLTMDIPFKKQEEFVEGRVLGVDLGIAVPAYVCLSDDTYKRKPIGSYEDFFKVRKQFKERRERLQRQLKMVKGGKGRNKKLQALNSFREKERNYAKTYNHNISKMIIDFAKQHKCKQINIENLSGNSFDEKILSSWSYYELQQMITYKAERIGINVKKVEASYTSQTCSRCGHIDKENRQTQDRFKCIKCDFELNADHNAAINIARSNKYVK

SEQ ID NO: 422 - protein, 403 aa, unidentified
  source 1..403: note = uncultivated organism; MG119-126 Effector
MAITVRKLKLTIVNGDENVRREQYEFIRNSQYAQYQALNLGMGYIMTGYLACNRDIKSEDFKEWQKGLKNSNPIFKGIEFGKGVDTLSAVTQKVKKDFSTSLKNGMAKGERASTNYKRSFPLMTRGRNLTFYEENGEIYIKWVNKIVFKVVFNARKENTLELQHTLNKVITGEYKLSQSSLEFDRNNNLILNLTMDIPFKQQEEFVDGRVLGVDLGVAVPAYICLNDDTYKRQGLGSYEDFFKVRQQMKARRERLQKQLKLVKGGKGRNKKLKALNQFKEKERNYAKTYNHNLSRAIVDFAKQHKCGQINIEHLTGESFDNKLLGSWSYYELQQMIAYKAERIGIEVKKVNPAYTSQTCSRCGNVDKENRQTQEKFKCTKCEFELNADHNAAINISRSKKYVK

SEQ ID NO: 423 - protein, 530 aa, unidentified
  source 1..530: note = uncultivated organism; MG119-127 Effector
MSKKGKKDKKNKKIVLCRKLKLTILGDEQTRKEQFKLIRDDQYQQYKALNLCMDLYQANHALRKYEFKLEVDSRKELNDSIPKKEKLIEKILTLEKQIISLEEKGKLNEKDEEKLEQKKSSLNKKNDELSALEEKILKLKNNIKNSIEYQASLSEKMQEKYIDDIHTVVQNQVNLNYKDTLSLVTQKVKEDFKIALNNGLKEGKRKSTSYKRDFPLMTKGRELKFKYRDENSDDIIIKWVKGITFKVILSNKKNNDKELRHTLHKIINNEVKICDSSLQFDNKNNLILNLAMEMKVERGFTPIEGRVMGVDLGMKIPAIASFNNMNGFKKFGSIDDFLRVRKQIQARRRKLQIALTTVSGGHGRDKKIQALDRFSKSEKNFVNTYNHTISKKIIEYAKVNKCGVINLEFLSLADQEKDLFLTMSESKIKQSRFIKRNWSYFDLQTKIIDKANREGIKVLLVDPYHTSQTCSKCGHYETGQRTSQAHFKCKSCGYEANADFNASQNIAKSTQYIDKKEDSQFFKNKKSKAS

SEQ ID NO: 424 - protein, 493 aa, unidentified
  source 1..493: note = uncultivated organism; MG119-128 Effector
MGKMDIKEEKIFRASNKVKITIVEDDPDERNRKYKLIHDEMYKQYLALNMCMSLLATQNTLRMYNSGAENRLLSQIAKLDKKISEKQQIADKDVNTEKEQTRKQKAIEDIAKLEEEKNKLREEFDKKATNRTDIDVKFHEQYTSDLYQIIRNEFDFASSITAGLVARRAKDDFKNSFSMGYAKGERSLISYKRSFPLMIRGNEDACYDSNKRKWLDFYYDGDDIFIRWLDGFIFKAILGNNPNRNMEIKHTLHKIITGEYRVSQSSIEFDDKGHMYMNVSYRFKKNSKTEIIPGRTLGVDLGIATPAYVCLSDTTYVRSGIGSANDFFRIRQQMKSRRRSLYKSLSLVKGGKGRKKKLQAINSLRDKERNFAKTYNHQLSNKIVKFAKDNGCEFINLEHLTKNGFEDAILSSWSYYELQNMIEYKAEREGIKVRYVDPAYTSQTCSKCGHVDKENRPTQAKFKCINCGLELNADHNASINIARSDNFVNKKKK

SEQ ID NO: 425 - protein, 416 aa, unidentified
  source 1..416: note = uncultivated organism; MG119-129 Effector
MINVRKLKLVIRETDKELRSQKYKFIRDSQYAQYRALNVAMGYISSAYLKANKDIKSEEYKEAIKLLTNSNPIFNGIEVGTGIDTLSAVTRKARKDFSASLKNGLARGERNLTSYRRTYPLITRGRDLKFRYEGDDIVIKWVNKIEFTVITGSNNMKENTIELKHTLHKIINNEYKIKESSLMFDKNNNLILNLTFDIPDKEIHDIVPGRTLGVDLGIATPAYVCLSDETYIRSEFGSANDFFRVRQQMKDRRKKLYASLTVVKGGKGRNKKLKAADRLRDKERNFVKTYNHQLSHKIVKFAKDNKCEFINLEYLTKDGFENRILSSWSYYELQNMIEYKAEREGIKVRYVDPAYTSQTCSKCGHVDKENRPTQAKFKCVNCGLELNADHNASINIARSDNFVNKKKKKKEDKQEE

SEQ ID NO: 426 - protein, 403 aa, unidentified
  source 1..403: note = uncultivated organism; MG119-130 Effector
MAITVRKLKLTIVNEDENVRREQYEFIRNSQYAQYQALNLGMGYIMTGYLACNRDIKSEDFKEWQKGLKNSNPIFKGIEFGKGVDTLSAVTQKVKKDFSTSLKNGMAKGERASTNYKRSYPLMTRGRNLTFYEENGDIYIKWVNKIVFKVVFNARKENTLELQHTLNKVITGEYKLSQSSLEFDRNNNLILNLTMDIPFKQQEEFVEGRVLGVDLGVAVPAYICLNDDTYKRQGLGSYEDFFKVRQQMKARRERLQKQLKLVKGGKGRNKKLKALNQFKEKERNYAKTYNHNLSRAIVDFAKQHKCGQINIEHLTGESFDNKLLGSWSYYELQQMITYKAERIGIEVKKVNPAYTSQTCSRCGNVDKENRQTQDKFKCTKCEFELNADHNAAINISRSKKYVK

SEQ ID NO: 427 - protein, 403 aa, unidentified
  source 1..403: note = uncultivated organism; MG119-131 Effector
MITVRKIKLTVIGTDEERKQGYKFIRDSQYAQYQGLNLAMGILSSAYLESNRDIKSDIFKNAQKSLTNSNPLFEGITFGTGIDTKSSITQRVKKDFKTSLKNGLAKGERTITNYKRNYPLMTRGRDLKFMYEGDDILIKWVNKIIFKVVLGATKRKENSIELQHTLHKVIDKEYKVMQSSLCFDKNNNLILNLTLDIPYTPKKNFVEGRVLGVDLGIKYPAYVCLNDDTYKREHLGSIDDFMKVREQMQQRARNLQQALKLTKGGKGRTKKTQALNRLKDKERNWAKTYNHQLSKKIVDFAIKHQCEFINMEKLTKDGFGDKLLRTWSYYELQNMIEYKAERVGITVRYIDPAYTSQTCCICGHIDKDNRQTQEKFKCTECGFELNADHNASINIARSEKFIK

SEQ ID NO: 428 - protein, 403 aa, unidentified
  source 1..403: note = uncultivated organism; MG119-132 Effector
MITVRKLKLTVIGTDEERKQGYKFIRDSQYAQYQGLNLAMGILSSAYLESNRDIKSDTFKNAQKSLTNSNPLFEGIAFGTGIDTKSSITQRVKKDFKTSLKNGLAKGERTITNYKRNYPLMTRGRDLKFMYEGDDILIKWVNKIIFKVVLGATKRKENSIELQHTLHKVIDKEYKVMQSSLCFDKNNNLILNLTLDIPYTPKKNFVEGRVLGVDLGIKYPAYVCLNDDTYKREHLGSIDDFMKVREQMQQRARNLQQALKLTKGGKGRTKKTQALNRLKDKERNWAKTYNHQLSKKIVDFAIKHQCEFINMEKLTKDGFGDKLLRTWSYYELQNMIEYKAERVGITVRYIDPAYTSQTCCICGHIDKDNRQTQEKFKCTECGFELNADHNASINIARSEKFIK

SEQ ID NO: 429 - protein, 403 aa, unidentified
  source 1..403: note = uncultivated organism; MG119-133 Effector
MAITVRKLKLTIVNEDENVRREQYEFIRNSQYAQYQALNLGMGYIMTGYLACNRDIKSEDFKEWQKGLKNSNPIFKGIEFGKGVDTLSAVTQKVKKDFSTSLKNGMAKGERASTNYKRSFPLMTRGRSLTFYEENGEIYIKWVNKIVFKVVFNARKENTLELQHTLNKVITGEYKLSQSSLEFDRNNNLILNLTMDIPFKQQEEFVEGRVLGVDLGVAVPAYICLNDDTYKRQGLGSYEDFFKVRQQMKARRERLQKQLKLVKGGKGRNKKLKALNQFKEKERNYAKTYNHNLSRAIVDFAKQHKCGQINIEHLTGESFDNKLLGSWSYYELQQMITYKAERIGIEVKKVNPAYTSQTCSRCGNVDKENRQTQEKFKCTKCNFELNADHNAAINISRSKKYVK

SEQ ID NO: 430 - protein, 467 aa, unidentified
  source 1..467: note = uncultivated organism; MG119-134 Effector
MITVRKLKLTIVGDEETRNQQYKIIRDEQYQQYRALNLCMSLLNTHNILNTCNSGAENKLNSQIEKLDKKIEKNKNDLKKDNLKDNKIKKINESIQTLTKEKEKLQQEYLKSSKYRSNIDKKVKEMYIDDLYTVVQSQVNFKSKDMMSLVTQRAKKDFATALKNGMAKGERSLTNYKRDFPLMTRGERWLKFEYDEDSDDIYINWLHGIRFKVVLGYKKNENSIELRHTLHKVINKEYKICDSSMQFDRNNNLILNLTLDIPLNIKNEHIEGRILGVDLGIKYPAYVCLSDDTYKRKSIGCAEDFIKFREQIRARRYRLQKQLSMVKGGKGRNKKLQALDRIKDKERNFVKTYNHMISKNVVEFAKNHKCESINLEKLTKDGFPNMILSKWSYYELQNMIEYKAEREGISVKYVDPAYTSQTCSKCGHVDKENRTSQEKFKCIECGFELNADHNAAINIARSNNYVK

SEQ ID NO: 431 - protein, 403 aa, unidentified
  source 1..403: note = uncultivated organism; MG119-135 Effector
MSITVRKLKLIIVNDNEEERKAQYKFIRDAQYSQYQALNLGMGYLMTGYLAYNRDIKSEEFKAWRKELKNSNPIFKDVKCGKGIDTLSAVTQKVKKDFSTALKNGMAKGERTSTNYKRTFPLMTRGRDLKFYAEGSDIYIKWVNKITFKVVFNARKENTIELQHTLHKIINEEYKLSQSSIEFDRNNNLMLNLTIDMPFNECDNLIKDRVLGVDLGIKVPALVCLNDDTYKRQSIGSYEDFFKVRQQFKSRREKLQRQLKMTKGGKGRKQKLKAFNKFKEKERNYVKTYNHNISKCIIEFAKKHKCSQINIENLTGENFDSKVLGSWSYYELQQMITYKAERVGIIVKKVDPAYTSQTCSVCGHIDKENRQTQEKFKCTMCGFELNADHNAAINIARSNKYVK

SEQ ID NO: 432 - protein, 145 aa, unidentified
  source 1..145: note = Uncultivated organism; MG119-1_sgRNA1
AUAGGGCGACUUGGCGUCCUUAAACCGAGAAGUGCAUCAGAAGGUUUACAUAGAUGUAACACUCGGUAAGGUAAAUCCGUAAGGAUAAUCCACCCCGGCGAAAUGCCGGCCACUAAAAUGCAUAGAAAUAUGCACAAGUGGAAGU

SEQ ID NO: 433 - skipped

SEQ ID NO: 434 - RNA, 129 nt, unidentified
  source 1..129: mol_type = other RNA; note = Uncultivated organism; MG119-2_sgRNA1_Mutant1
tacacatcattagatgtgagggcaatttaatgtcctaatattgagggtattaggtaacaacactttgttctaatagaacagtgtctaataaccactcgataagaccatatgaaagtatggttataaaac

SEQ ID NO: 435 - skipped

SEQ ID NO: 436 - RNA, 156 nt, unidentified
  source 1..156: mol_type = other RNA; note = Uncultivated organism; MG119-3_sgRNA1_Mutant1
gttaaaattctagatatgattattatattccctttatgggaagggtgtgttggaacgccttagtttgaggtttgaatataaaaagtgaaacaaatggagatttgtaacaccttcaaacactcaaaaaattcatttagaaataaatgaatatccaac

SEQ ID NO: 437 - skipped

SEQ ID NO: 438 - RNA, 117 nt, unidentified
  source 1..117: mol_type = other RNA; note = Uncultivated organism, MG119-4_sgRNA1
ttagaaaagaaattttctaattggggctgctggaagtccttaacctgagataggaaactatactcgggaagtacgtcaaaacattgagaaatcattgttttgaatggtatatccaac

SEQ ID NO: 439 - skipped

SEQ ID NO: 440 - RNA, 143 nt, unidentified
  source 1..143: mol_type = other RNA; note = Uncultivated organism; MG119-10_sgRNA1
tattgcgatgagtaaaaaaatcgtagaaagataattagagaagaaattttctaattggggctgctggaagtccttaacctgagataggaaactatactcgggaagtacgtcaaaacagaaatgttttgaatggtatatccaac

SEQ ID NO: 441 - skipped

SEQ ID NO: 442 - RNA, 132 nt, unidentified
  source 1..132: mol_type = other RNA; note = Uncultivated organism; MG119-19_sgRNA1
caagtagatgcgaaagataaagtatgtaagttttgaataacaatattattcaaattggggcagttggatgcccttatgctgagggattattccactcggcaagtaccaatagaaatatgaggtacatccaac

SEQ ID NO: 443 - skipped

SEQ ID NO: 444 - RNA, 157 nt, unidentified
  source 1..157: mol_type = other RNA; note = Uncultivated organism; MG119-27_sgRNA2_Mutant2
ttgatataacgtatatgaatcttctattcccctgtgggggaagggtgtgttggaacgccttggtttgaggtttgaaatagaaaaaagaaatacgggaatgtattatcgatttcaggcactcaaaaaattcacttaagaaattaagtgaatatccaac

SEQ ID NO: 445 - skipped

SEQ ID NO: 446 - RNA, 152 nt, unidentified
  source 1..152: mol_type = other RNA; note = Uncultivated organism; MG119-28_sgRNA2
ttgaaataaaatgaatttcaaaccccttcgggggagggcgcgttggagcgccttagtttgaggtgcagaatcaaaaaaactgcgacgatggaggtcgtttcagtctctgtacactcaaaaaattcacttgagaaatcaagtgaatatccaac

SEQ ID NO: 447 - skipped

SEQ ID NO: 448 - RNA, 140 nt, unidentified
  source 1..140: mol_type = other RNA; note = Uncultivated organism; MG119-32_sgRNA1
atatagtcccattagggattgggcgtgttggaacgccttagtttgaggtttgaaaccaaaagcggtgtagatggaggtctgtaccaccttcaagcactcaaaaaatccacttatatgaaaatataaggggatatccaacg

SEQ ID NO: 449 - skipped

SEQ ID NO: 450 - RNA, 147 nt, unidentified
  source 1..147: mol_type = other RNA; note = Uncultivated organism; MG119-54_sgRNA1
aacagatatacgacacgaagtatatttacatggggctgttggaggcgccttagtttgaggttcagacataaaaaatcgttatgacagtcggttataacgatgtctgcgcactcgaaaaattcatctcgaaagggatgaatatccaac

SEQ ID NO: 451 - skipped

SEQ ID NO: 452 - RNA, 112 nt, unidentified
  source 1..112: mol_type = other RNA; note = Uncultivated organism; MG119-64_sgRNA2
ggggcagttggatgcccttagcctgaggaatttatttcacttgggaagtaccattcttattgaatgacagctagttatgttactgaaaagtaaaatgtttggtatatccaac

SEQ ID NO: 453 - skipped

SEQ ID NO: 454 - RNA, 133 nt, unidentified
  source 1..133: mol_type = other RNA; note = Uncultivated organism; MG119-72_sgRNA1
aaatatttcattaagtaccgaatttaaaaaataggattgcagaaagtgcaattaggctggttgtgcagccttaatctgagggattaatccactcggaaagtacctttattgaaaaatgaaaggtattcacaac

SEQ ID NO: 455 - skipped

SEQ ID NO: 456 - RNA, 104 nt, unidentified
  source 1..104: mol_type = other RNA; note = Uncultivated organism; MG119-83_sgRNA1
aaggaataggtcataaggcctatggggctgttggaagcccttagcctgaggactttagtcactcgggaattaccttaatttgaaaaaatgaaggcaaatccaac

SEQ ID NO: 457 - skipped

SEQ ID NO: 458 - RNA, 156 nt, unidentified
  source 1..156: mol_type = other RNA; note = Uncultivated organism; MG119-97_sgRNA1_Mutant1
tcatggggtgatttggcatcctacaatcgagatagtgtatataagactatattaatatagcaataaaaacactcgataaggctctaagaattctccaaccgaattcttaaccaccttgtccgaaaaggacaacaatatatgaaaatatattgaaag

SEQ ID NO: 459 - skipped

SEQ ID NO: 460 - RNA, 155 nt, unidentified
  source 1..155: mol_type = other RNA; note = Uncultivated organism; MG119-109_sgRNA1
attcagggcggctcgacgtcctaaaatcgagaaagtatgtataagtctttataatatggagcagcagatactctcggtaaggtataaattccacaatgtaaatcctgtggagaccctggttattgtgtagaaatgcgagagtgtgccgggtacga

SEQ ID NO: 461 - skipped

SEQ ID NO: 462 - RNA, 170 nt, unidentified
  source 1..170: mol_type = other RNA; note = Uncultivated organism; MG119-118_sgRNA1_Mutant2
cggcagggcgacttagcgtccaaaagtcgagtgatttgacgataatatgtcaaactttaatagtaatagtttactctcgataaggtaacaaattataagatttgttgaaactgcatatataaaatccatatgcacatctcgtatttgaaaagatatgaaatgtaagttga

SEQ ID NO: 463 - skipped

SEQ ID NO: 464 - RNA, 141 nt, unidentified
  source 1..141: mol_type = other RNA; note = Uncultivated organism; MG119-121_sgRNA1_Mutant1
attgagggcgactgtgcgtcctaagtgtgagggaatttaccaagtgaatatccactcataaagtcttgaatatattgatattacaagacatcaccctgcaagtagcaggtataatactctagaaatagagtattataagta

SEQ ID NO: 465 - skipped

SEQ ID NO: 466 - RNA, 144 nt, unidentified
  source 1..144: mol_type = other RNA; note = Uncultivated organism; MG119-125_sgRNA1
taaacaattgaataaaaaatattatatacgggcgattcaccgtccgaaggttgaggatacgtaagaatataagtctaggctttggccaacagatttccgtatcactcgataaatcacatagtgaaaactatgtggtatgtgaat

SEQ ID NO: 467 - skipped

SEQ ID NO: 468 - RNA, 135 nt, unidentified
  source 1..135: mol_type = other RNA; note = Uncultivated organism; MG119-128_sgRNA2_Mutant1
ttagttacacatcattagatgtgagggcaatattatgtcctaatattgagggtattaggtaacaacactttgttataatagaacagtgtctaataaccactcgataagaccatatgaaagtatggttataaaact

SEQ ID NO: 469 - skipped

SEQ ID NO: 470 - RNA, 133 nt, unidentified
  source 1..133: mol_type = other RNA; note = Uncultivated organism; MG119-129_sgRNA1_Mutant1
tacacatcattagatgtgagggcaattatatgtcctaatggtgaggatgcagattaaagcattaggttcatgaaccaacaatgtactgtaatcactcgctaaatcgtgtaagaaattacatggtactaaaaca

SEQ ID NO: 471 - skipped

SEQ ID NO: 472 - RNA, 144 nt, unidentified
  source 1..144: mol_type = other RNA; note = Uncultivated organism; MG119-133_sgRNA1_Mutant1
taaataaattaaaaatacatattatatacgggtgatttaccatccgaaggtcgagggtgtgggtttaatatctgggtttagaccaaaaggattctcacatcactcgataaatcatatggttgaaaaactatgtgatatgtaaat

SEQ ID NO: 473 - skipped

SEQ ID NO: 474 - RNA, 127 nt, unidentified
  source 1..127: mol_type = other RNA; note = Uncultivated organism; MG119-136_sgRNA1_Mutant2
attataagttatattataattgggctagttgcatagcctttcgtgaggttaacgaattaaatatattaattcgttgaaaaacactcataaactactgtaaattgaaaaattgtatggtagatgcaac

SEQ ID NO: 475 - skipped

SEQ ID NO: 476 - protein, 464 aa, unidentified
  source 1..464: note = Uncultivated organism; MG119-136 effector
MAVKAAKIELEVCDVNKRNSFIESVGKHINNTFKSQYILTLKENKKDIYDDLLKLQKSNFRLHNFIVNKYQNFINSDDKKFKLENNEKNEINHLATKYSKKLCEEYQLNNINSTIPNGILNLIDKNVKKTIKDIKNGKRIWQSYREQVPIPLSQKDAIVKINGKHFTKIDEMNKNYYIVFWGILFKTNLGRDKSNNASIIEKTISGEYKFCDSNVVVEKNKMFLLLSYDAPQKQIELDDDKVLGIDLGIKIPAVGCIYSKTLGKPITYQHNGKDVIESFDETYVKSKYVNKYEKKEIKPSILTIRNAIKIEHEKRKKDAKYICTNGHGRNRKMKFADKISNKEKNTANLYYHNLSRDIINFALKNKCKQINIENLKGFKGDKVLINGWGYFTLQNQIEYKANEYGIKVKKINPKNTSRTCSSCGHVSEKNRIDQEHFKCEHCGLEMNADYNASINIAKSENFVK

SEQ ID NO: 477 - protein, 446 aa, unidentified
  source 1..446: note = Uncultivated organism; MG119-139 effector
MGVSCKKLQIIPVGDDNEIKRVYEYIRNGIYSQYRILNTYMSQVGTLYYKYDRDYKSPGFKDEYNAIFRNTNTAIHDIEQAKGLGMAGNCGMRVKSDFSTALKNGLARGERQLPFYKRDFPLLVPGRFLVFSEKEEDYYDDNGELKTVVNYYIKFVNGIVFKVVFGSKGRRDYRLTSLLSGIVNDVDHYKVCGSSIQISGKKIILNLTTNVEKETVPFIPIKDKKMGIVLGYDQPLVAALSNDDKEYYIGTDIVESIVERRTAIQAYRHRLQAMQTTAKSGHGRKRRMRRVDQQGHYERNVMRNYNHILSSRVVSFAVKNKVETIFIDDISKDTLVDEPILLRNWSYYQLVEFITYKAKEVGITVITVDFKKNKKEDGEDDTDASNKHDFFDRCCVCGEQIDGLAELYDDISVYKDESSFTCPHCHKKVKVRYNKAKNLTYYKNKK

SEQ ID NO: 478 - protein, 525 aa, unidentified
  source 1..525: note = Uncultivated organism; MG119-140 effector
MAEKMTMITKKCVLIPERSDSSLWERRVYDFTVKDYEQRISFYESQLESKNKEVKRNAKTRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVAIGLVQPAVCALNNDQYVRLPIGDERDLLDNKLQIQERRRRLQSALRETSGGHGRKKKMQALNRIAKTESNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKSMDEAAEYYGISEKYKA

SEQ ID NO: 479 - protein, 525 aa, unidentified
  source 1..525: note = Uncultivated organism; MG119-141 effector
MAEKMTMITKKCVLIPERSDSSLWERRVYDFTVKDYEQRISFYESQLESKNKEVKRNAKTRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTNYKKGFTADFNHARNVAMSTLFMENGSFSKKSMDEAAEYYGISEKYKA

SEQ ID NO: 480 - protein, 535 aa, unidentified
  source 1..535: note = Uncultivated organism; MG119-142 effector
MAAKDNTMVITRKIALIPVVSERKEWKKRINAFLEKDFPRKIEAKKKQIKNTSKPENKDGYKLQLAELEKQYEDFKENRIEEYTQKMVNDYTYGLVRDAMESEARRKNYILSYIYTKMVQDGVGYMPTLKDKYTWIKENINVAYRRKGSNKGSIFDDVDIDNPLGGYGTCFSQILTSKIKDLVKQGLLDGKVSIPSFKIDSPFTIAKAYFDLYHEYEDIAELKKRIYDSDCKLYCGLGNKGIPTIANFKVNIGHKGNRDELISTLLRIFTGEYEICGSTMQISKTKIILNLSIRIPKTQLELDEDTVVGVDLGVAIPAVCSLNNNKYKREYIGSSDDFIRMRTKLQHEKQQLQKALKMSKGGHGRKRKLLALERCKKKEHNFVETYCHKVSKKVVDFAVKNKAKYINVENLSGYDSSKFILRNWSFYKLQQYIEYKAAKYGIEVRKINPSFTSQVCSFCGHWEDGQRKDQATFVCGNPNCESHKLHMVNADYNASRNIAMSTLFTSDKFQFCKKTMQNAAEYYGIQLDNEDQLAA

SEQ ID NO: 481 - protein, 552 aa, unidentified
  source 1..552: note = Uncultivated organism; MG119-143 effector
MINKDKNENTVTITRKYALIPVSSDKTQWYKKVYDFTIEDLTRKIEYYKEKLKKEKDKEKKEQIKTKIKNFEEGLESIYNGGDFTQKMINDYTYNLVRTAMEEEARRKNYILSWIFSEMRLNRVDQMESLKDKFKFISDTLNYAYRKKGSNKGSLFDDTEINSILNAYGIAWNQELTKEIKDLVKNGALEGKVSLTNYKLDSPFTIAKAHFSFDHDYDSFEELCEHIDDSDCKMYMNYGGDNKKDGTNPASLARFRINLGQGKNRNELKSTLLKVYSGEYQYCGSSIQISKNKIILNLSMKIPKIETELDENTVCGVDLGIAVPAMCALNNDMYKRLAIGSADDFLRVRTKHQEQRRRLQKSLKNSNGGHGRKKKLKPLERMNKAEAHFVETYCHMVSKRVVEFAVKNRAKYINIENLNGYDSSEFILRNWSFYKLQQYITYKAARYGIVVRKINPCYTSQICSVCGNWEPDQRKTQAKFECANEECASYDKYKYGFNADFNAARNIAMSTLFMETGEVTEKKKEEARKYYGIEEKYQKYQASLKEKDDKVA

SEQ ID NO: 482 - protein, 579 aa, unidentified
  source 1..579: note = Uncultivated organism; MG119-144 effector
MKKNNKESNTYIKAMKIKIIPICDIKEENLRDKYYKELYQYLRDSIWAQNAALNYGLAMTRDAYVLHKNEDKIKDIYFKLAHQTPNPSMASEARLKEVYEAAPITQEYIDNKVEEFKKSNSKKKKPLTDEGVKKKTDSINNMYKKFIGLSKEDIQELIDKLDDYCAYPEEIYEKFANGFSTPAYVTQQIKDYWNTEGVMTKVIYSMSDKLRSVKNSNPLTIPPNLFYNKKNDLIGITHSYNTYLEFVEALMNAYDANLFLELPYKKGYDKMKFKLILGNPCKSHEVRISLQKIFEEYYKIRGSKIGFVRNKKTGKNTDLVLYLSVEIPKDTSIELDENTVVGVDLGLAVPAVCALNNNPYPRKYIGKKLELLKKRTQFKNRRKKLQSELRDARGGHGRRRKMKAMDRLSELEKNFADTYCHKVAKKVVMFALKNKAKYINLEMLKGYRADEKVLQNWSYYRIQMYIKILAERYGIIVRFINPCYTSQVCSVCGNWHPGNRPKGDKGQKYFDCHNDKCESHTKNKGKGYTLNADYNAARNIAMCDLFMDNKSEITEEDKKIAREKYGIPEPVKKEEKIAA

SEQ ID NO: 483 - protein, 535 aa, unidentified
  source 1..535: note = Uncultivated organism; MG119-145 effector
MTAKDNTMVITRKIALIPVASERKEWKKRINSFLEKDFPRKIEAKKKQIKNTSKPEKKDGYKQQLTELEKQYEDFKENGIEEYTQKMVNDYTYGLVRDAMESEARRKNYILSYIYTKMVQDGVGYMPTLKDKYTWIKENINVAYRRKGSNKGSIFDDVDIDNPLGGYGTCFSQILTSKIKDLVKQGLLDGKVSIPNFKIDSPFTIAKAYFDLYHEYEDITELKKNIQNSDCKLYCGLGNKGIPTIANFKVNIGHKGNRDELISTLLRIFTGEYEICGSTMQISKTKIILNLSIRIPKTKLELDEDTVVGVDLGVAIPAVCSLNNNKYKREYIGSSDDFIRMRTKLQHEKQQLQKALKMSKGGHGRKRKLLALERCKKKEYNFVETYCHKVSKKVVDFAVKNKAKYINVENLSGYDSSKFILRNWSFYKLQQYIEYKAAKYGIEVRKVNPSFTSQVCSFCGHWEDGQRKDQATFVCGNPNCESHKLHMVNADYNASRNIAMSTLFTSDKFQFCKKTMQDAAEYYGIQLNNEDQLAA

SEQ ID NO: 484 - protein, 522 aa, unidentified
  source 1..522: note = Uncultivated organism; MG119-146 effector
MENNITITRKYALIPEFSDRKEWKKRVYDFMINDLEQKIDYRNKKKQDTSELESQLEYIKNGGDFTRSMVNNYTYSLVRKAMEEETRRKNYILSWIFSEMRANRIDQMESLKDKFKFVSDTINYAYRKAGSNKGSLFDETEIHCILKSYGIAFSQELTKEIKELVKNGVLEGKVVIPTYKLDSPFTIAKSHFSFEHDYDSFEELCEHINDSDCKMYMNYGGDNRKDGINPASIARFRISLGHGKNKDELKSTLLKVYSGEYQYCGSSIQITKNKIILNLTMKIPKIETKLDENTVVGVDLGIAVPAMCALNNNMYERLAIGSADDFLRTRTKLQSQRRRLQKSLKNSNGGHGRNKKLKVLERLGKSETHFAETYCHMVSKRIVDFALKNNAKYINIENLNGYNTSSFILRNWSFYKLQQYITYKAERYGIVVRKINPCYTSQVCSVCGNWEDGQRKTQASFECANPKCESHKKYKYGFNADFNAARNIAMSTLFMEDGEVTEKKKEEAREYYGIKKENSEAV

SEQ ID NO: 485 - protein, 521 aa, unidentified
  source 1..521: note = Uncultivated organism; MG119-147 effector
MAKSSENVLTRKVQLIPEISDRKEYTKRLTKYINDSYSEKIEKKRNKKFNRKDYDSLEKFKEAKEKNESEIQNLQDALADLATGTITKKMVDDYTYGLVREAMESESSRKNYILSYVYSQLITSDYANMDKEERKQFFKDIWNPCCRKKGSNLGSIFDNTDIDNILGGYGTGFSQDLKSKINNAIKDGLLEGKVSLPMYKKDSPFSIASKFFNFYHTYESFDELEKNIEKPNCQLFMAFGGNGNPTIANFRVNLGCGSGAKKRKDNSICQTMLKVLAGEYKICGSSIQISKGKIMLNLSMDIPIKEHKLDPDICLGVDVGYVIPAMCGLNKGKLAFRDARFGDGTDLNGRRKRIREQKKRMQVGIATNKGGHGRAKKIKALDRFREKERNFARTYNHKVSHDIVNLALKNNAMYINLENLKGITSDEKKKRELGDWSYFQLQDFITYKAKQHGIIVRKVNPYRTSQTCSCCGHWEEGQRVDRDTFICKNSECEQFGKKIHADLNAAINIAKSTDFSVKKDA

SEQ ID NO: 486 - protein, 576 aa, unidentified
  source 1..576: note = Uncultivated organism; MG119-148 effector
MNTINNTYIRTLKFNLNLTPKFETEEDNKKYINDVYSYLRDAMWAQNRAMNIVLDRTKEAYTLGRGMNRVKEIYYSYSHQKPISNDKKESFLESLLQYAPIDDQFVKNEVKKLRKFYESKKKPPKEETVSKNCEALKNKYIKYVGKSKDDIKRELDLLENYCAYPEDIYEKFANGLSTPAYIKQKVESYWKQDGIKTKVIYSMDENLRRIKDAPLFIPPNIFYNKKDELIGLVYDYTDYISFLEDLENKRNVNIYLSIPYKKGEDKLKFKLVLGNPHKSRDSRLSIKRIFEEEYRIKGSSIGFTKNKETGKNTNLTLYLTVEVPQNKDNTLDENVVVGVDVGIAIPCVCALNNDKYTRENIGSYDTLFAKRTQFKMQRSRLNSQLKLSKGGHGRKRKLKKLELLSGKEKNYADTECRKYASDVIKFCLKNHAKYINLEHLKGYRENPKVLAGWSFYKIQTYIEQAAEKHGIIVRKINPCYTSQICSVCGNWHPENRPKGKLGQAYFNCHNIDCKTHNTDLYKYGINADFNAARNIAMSTLFITDSDEITKKHWKEAREYYGIDESDDKEEKLNKVA

SEQ ID NO: 487 - protein, 675 aa, unidentified
  source 1..675: note = Uncultivated organism; MG119-149 effector
MSKKNTIVEITFTNGVKKEVVLPPKASTIPSKKIRMIEQEKNYGIRKVVIPEGITKITSRSFKDILSLEEVVLPDSLTVVGNSAFQNCISLKIIKIPNSVTAIGNSAFQNCAELTDIDIPDSVTVIGHSAFQNCIGLISVTIGSNVTEIGDYAFSGCSGLTGIDIPHSVNEIGSYAFNDCTGLVSITIPNSVLRIGNGAFEGCTGLTGTENSSNRTNGNVVIISRKIQIVPIGDTKNCAPIHEYISEAMDAQYRAKNLLMGHIGSMYYKNEQKMTKDLKSGVLYSGNPVLSSISFPIGLDLNSSIRNQIRQEIDSALKNGVAKGERSLSSYKRNNPVPTNGRNVHFNFEFDEKDIENYKDDPTNPLNEKNLLMHMRVTINWVNNKGFIVNPGSSMDIRRILFRLYTGEYKHGQAKLQILKDTNDKKNLEKIMMILSLVVPNTSIELDENTVVGVDIGAKNPAVCALNTDENKRLYCGDNFAMLKAHTEFQGRKKEIAHTSLFNCSQHGYRKRNKRLNQAKKHEQNKTKNISHEIAKQVVEFAIENNAKYINVENLEGLFETPGNSADRKVWSYYQIQTLIMQKAARYGIEMRKVESRNTSRICSFCGSDAEDQLRDRDTFICKNPDCVSHKLHHGFVESDFNAARNIAKSTEWSKGKAKKSSKEKDKNKDESKKQ

SEQ ID NO: 488 - protein, 512 aa, unidentified
  source 1..512: note = Uncultivated organism; MG119-150 effector
MDTMIITRKFVLVPVASCKKEWVKKATAYIVSDTEDKLDFFKEKLKKVEKEMKKAKDSEQLSKEYNRLCSKKEEYESLLKDAEDGVFNQKAINNYTYHLVREAMESEARRKNYILSWAFSEMIANGVPYMETYKEKCKFINEMIKPAYRTKGSKKGSLFDDSEIQNILGGYGVSFKQELTQKLKDCVKDGLLEGKVVLPNYKLDSPFTVEKTHVSLSHDFETYEELCEHIGKKDGKIYLNYGGHGESTIAKFAIDTGSNKNKEELNTTLLRLISGAYEVCGSSIGLCKDGKKIVMNLSMKIPKTELELDEKTVVGVDLGIAVPAYCALNNNIYSKKAIGSAAEFLRIRTQLQSQYRRLQKQVAANNGGHGRNKKMKPLDRFREKERNFVKTYNHYVSKEVVEFAVKNKAKYINMEDLSGFKGNDDRILRNWSYYELQQFIEYKAAKYGIEVRYINPYYTSQVCSCCGHWEEGQRMDQAHFVCKKCGTELNADYNAARNISMSTEFKKQKLAA

SEQ ID NO: 489 - protein, 525 aa, unidentified
  source 1..525: note = Uncultivated organism; MG119-151 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTENNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTNYKKGFTADFNHARNVAMSTLFMENGSFSKKSMDEAAEYYGISEKYKA

SEQ ID NO: 490 - protein, 587 aa, unidentified
  source 1..587: note = Uncultivated organism; MG119-152 effector
MLDKKTNATSSKIITITESQKLELYPLPCIEVKQWNGRVNENFDYQKDLLEKSIKRKKGWINNYEKRLNEAERKLKDYISNNHIDNEDKDKNIKALNKQINECNKKIRKYEKECKEHMEKINAYQKEQDRFNEIINHDNLTKDDTEFINKTAKNYTYSLIRRACKSEAARKNATLSYTYSECIRNNVADMEDFGDICSAIDEILKHCNRTRNGLSDDLNIDNPLGGYGISYNQNLRSSFINMVKKGLFDGRVSLVNYKSDSPVSIAGAHYNFFPSFDLIGFEDFYDLKRVMQKENGVIYLNVGSNGLPSIARFRVVIGSKNHKSDSIYAFLYNVLKNNYPTGGSSIQIENNKIMLNLSYKKSSQEIDTLKDDVVVGVDVGISIPAVCALNNSKGYRWDIGDKKELLRIRTKLQEQKKRITNKKDLCSSGKEKKKINKASFRLTKHIKHWKRTYNHMLSRRIIKFAIKNNARYINLENISKFDKKEKERRFLKEWSYYELQKYIEYKAKEYGIIVRYVKAENTSTTCSCCGNTDEEQRNKSIFVCKNHECKQYGKKVNSDFNAARNIALSTDFFSKKELNEESLENED

SEQ ID NO: 491 - protein, 543 aa, unidentified
  source 1..543: note = Uncultivated organism; MG119-153 effector
MDSNNTQIVTRKLVIRPTFSERKEWKKKVTEFTLKDLEEQISKYKEWIENLKSNKKIDKEEKERRKQKYTDLLTNAENSLNQFKDREEITQKMVNRYTETLVKESMESEARRKNYILTWAYFSMRANGVQHMQPKEQKKFIREMLKPAYRVKNSSKGSIFDDVEIDNILGGYGMAFNQELTNKIIDSVEKGLFEGGKDSLPSYKYDSPFTVAKSTMGFSHDYDSFEELCEHIKEKDCNLYFDFGGNSDATIARFIIDVGTNRNKDELLITLCRVYSGEYEYCGSNIQFDKTGKKIILNLTIKIPQKNCDLNENTVVGVDLGIKIPAVCALNTNPYAREFIGSVDDYLRVRTQIQNQRRRLSIQLRNTKGGHGRKKKLKALDRYTSRERNFVKNYNHMVSSKIIKFALKYNAKYINLECLKGYDADKKALRNWSYYELQSMIEYKANKYGIVVRKINPCYTSQVCSECGHYEEGQRVSQEKFVCGNDDCRFHNTKSGYINADFNAARNIAKSELFLDSEVTKKRIEEAARYYNIPYESKNTDEV

SEQ ID NO: 492 - protein, 525 aa, unidentified
  source 1..525: note = Uncultivated organism; MG119-154 effector
MSEKMTMITKKCVLIPERSDSSLWERRVYDFTVKDYEQRISFYESQLKSKNKEVKRNAKTRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTENNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTNYKKGFTADFNHARNVAMSTLFMENGSFSKKTMDRAAEYYGISEKYKA

SEQ ID NO: 493 - protein, 534 aa, unidentified
  source 1..534: note = Uncultivated organism; MG119-155 effector
MSEKMTMITKKCVLIPERSDSSLWERRVYDFTVKDYEQRISFYESQLESKKKEVRENAKKRLEALNKELDAFLENPEITQNVINNYTYGLVHTAMSEESARKNYIIEYVKSILIAEHAYAMDFKDRNKRITQILQYAYRKKGSNKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFRIDLGHGRNRDKLMATLLKVYSGEYAYCGSSIQISKNKIVLNLSLQIPKEEKDELDEDTVVGVALGLVQPAVCALNNDQYVRLPIGDERDLLDNKLQIQERRRRLQSALRETSGGHGRKKKMQALNRMAKTEKNFTETYCHMVSKRIVEFALQNHAKYINMENLSGYNTDKFILWNSYASKIIMYTTYKAGHYGIEVRLVNPCFNAQVCSICGNWTPEQKISRTEFVCADPDCKSHTKYKKGFTADFNHARNVAMSTLFMENGSFSKKSMDEAADYYGISEKYKAHIGNEPKVE

SEQ ID NO: 494 - protein, 551 aa, unidentified
  source 1..551: note = Uncultivated organism; MG119-156 effector
MPKKKSNTKTKKDNKTKTPKVTSVVFCKKLKLKPVPVLDNKKWAKRVLAYFAYHINYQSELIEKYELWMQDATKTKKEAYKKQQAIAEKKIEKLKADAKRFENLFTNGLETQNDEDFIIKIAKDYTYDFIRKACKSEAQRKNAALSFTFSECLNRNIKDIEDIKERSRAISDILNFCNRVKSKSVSEQPNSLADMIDIENPLGGYGYDYKQAIRSNMLDMIKDGLLEGKIALKCYRMDGPVTIGADHFSITPDFDLIGTKDYTDLQRIIKKENGRLYVNIGGLGNPTIAQFEIILGSKQKARDDLFSLFYDILKKNCTFGGSSVQITDRHEIILNLTIKKDKELKNKLKDDVIVGVDVGINTPAMCALNKKKDFYLVLGSKDDFLRMRGQLKAQRKRLDCARRLCTTAEEKKRLLAKSERLTKHTDKWAQTYNHMISKRIITFALRNNAGIIRLENLSKFSDEQKQNNLLEDWSYFQLQNYIEYKAKAEGIKVEYVNPENTSTTCACCGETENTERRKNSIFICHNPKCKNCDKEVNADLNAAKNIASTEP

SEQ ID NO: 495 - protein, 533 aa, unidentified
  source 1..533: note = Uncultivated organism; MG119-157 effector
MDDDKVIITRKYTLIPEFSDRKEWDKRVRSFIKTDSENKIEYLIKKRTTVKGKAEKTKIDTRISTYKERIEIIDSGGEITQKMVNDYTYNLIRTAMEEEARRKNYILSWIFSEMRLHRVDCMETLKEKFKFISDTINYAYRKAGSSKGSLFDDTDITSILKSYGIAFSQQLTAEIKDLVKAGLLEGKVTLTNYKLDSPFTIARSHFSFDHDYDSFEELCEHVNDSDCKMYVNYGGDNTKTGKNPASIARFRINLGHGKNRDELKSILLKVYSGEYDYRGSSIQISGNKIILNLSMAIPKQTMELDEETIVGVDMGIAVPAMCALNNNLYEREAIGNADDFLRMRTKMQKQRRRLQKSLRNTSGGHGRKKKLRSLDRVGKSEAHFVETYCHMVSKRVVDFAVRHHAKYINVENLTGYDTSEFILRNWSYYKLQQYITYKAAKYGIVVRKVNPCYTSQMCSICGSYEPGQRKSQSVFECANENCNSHTKYKHGFNADFNAARNIAMTTLWMETGQVTEKSKQEAREYYGIAEEVA

SEQ ID NO: 496 - protein, 541 aa, unidentified
  source 1..541: note = Uncultivated organism; MG119-158 effector
MAKKQDDNTMTIARKITLIPVASDNNKWKKRVDNFLEKDFPRKIEAKKKQIKNTSKPERKDGYKQQLVELEKQYEEFKENGIKEYTHKMASDYTYDIVRKAMDSEARRKNYILSYIYTKMIQEEVANLPTLTEKNKWVSANVKECYRKAGNKNGSIFTNVDIDNPLAGYGSDFKQALTGKIKNLIKDGVLEGKVSVPNYKFDSPFTLTNQNFGIFTDCKDITELKKNIGKSTCPVYVCLGKNGSPTIAKFKINFGYKQNKNKAELISTIIKIFTGEYSVGGSTFGIDDDKIEMNLSITMQKQKMDLDENTVVGVDLGLAVPAVCALNNNEYDKQYIGSGNDLVSRRTKFQNEYTQLQKALRLAKGGHGRKRKLLALERLKEKENNFVDTYCHRVSKKVVDYAIKNRAKYINIENLKGYDSSEFALRNWSFYKLQQYITYKAEQCGIIVRKINPSFTSQVCSFCGHWEKGQRKDQATFKCKNPNCKSHKLYTVNADYNAARNIAMSTQFTDDKFKCSKETIQAAADYYGIVLEDDNNNDKAA

SEQ ID NO: 497 - protein, 525 aa, unidentified
  source 1..525: note = Uncultivated organism; MG119-159 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKNTVVGVALGLVQPAVCALNNDQYVRLPIGDERDLLDNKLQIQERSRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKTMDRAAEYYGISEKYKA

SEQ ID NO: 498 - protein, 552 aa, unidentified
  source 1..552: note = Uncultivated organism; MG119-160 effector
MKKNDDNKVIIARKYNIIPTCSEKKEWKKKVYDFTIEDLNKKIIDFEKKIKKQKDKKKKENYIQKLNTYKESLASLSVENPEFTQTMINNYTYNLIRSAMEEEARKKNYILTWIFSEMIANGVQYMPTLQEKNKFVSDTINYARRVKGSSKGSLFDNTEITNTLGAYGLVFTKELKNKIQRDIKDGLLDGKVSLSNYKLDSPCTIAKDHMKFTSDYDTFEELCEHIDDNDLKMYFNYGSKGEPTIAKFKINLGHGKNRAELKSTLLKVYSGEYEYCGSSIQIAKTKIILNLTMKIPKKELDVLNEDTVVGVDLGIKIPAMCALNNDAYSRQAIGSSDDFLVKREQFQKQRERLQKALKNTKGGHGRKKKLQALNRVKNAEAHFVETYNHMVSKRVVDFALKYHAKYINLENLTGYDTSDKILRNWSYYKLQTYITYKAKKYGIIVRKINPCYTSQICSVCGNWHPENRPKGDKGQGYFNCHNEECKSHDKEKYKYGLNADFNAARNIAMSTLWMDEGKVTEKKKEEAREYYGIEEEYQKYLEEKQKNKKKAA

SEQ ID NO: 499 - protein, 520 aa, unidentified
  source 1..520: note = Uncultivated organism; MG119-161 effector
MIPVASERKEWKKRINAFLEKDFPRKIEAKKKQIKNTTKPENKDGYKQQLAELEKQYEDFKENRIEEYTQKMVNDYTYGLVRDAMESEARRKNYILSYIYTKMVQDGVGYMPTLKDKYTWIKENINVAYRRKGSNKGSIFDDVDIENPLGGYGTCFSQILASKIKDLVKQGLLDGKVSIPNFKIDSPFTIAKAYFDLYHEYEDITELKKNIQNSDCKLYCGLGNKGIPTIANFKVNIGHKGNRDELISTLLRIFAGEYEICGSTMQISKTKIILNLSIRIPKTQLELDEDTVVGVDLGVAIPAVCSLNNNNYKREYIGSSDDFIRMRTKLQHEKQQLQKALKMSKGGHGRKRKLLALERCKKKEHNFVETYCHKVSKKVVDFAVKNKAKYINVENLSGYDSSKFILRNWSFYKLQQYIEYKAAKYGIEVRKINPSFTSQLCSFCGHWEDGQRKDQATFVCGNPNCESHKLHMVNADYNASRNIAMSTLFTSDKFQFCKKTMQDAAEYYGIQLDNEDQLAA

SEQ ID NO: 500 - protein, 525 aa, unidentified
  source 1..525: note = Uncultivated organism; MG119-162 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVAIGLVQPAVCALNNDQYVRLPIGDERDLLDNKLQIQERRRRLQSALRETSGGHGRKKKMQALNRIAKTESNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTKYKKGFTADFNHARNVAMSTLFMENSSFSKKSMDEAAEYYGISEKYKA

SEQ ID NO: 501 - protein, 525 aa, unidentified
  source 1..525: note = Uncultivated organism; MG119-163 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKNTVVGVALGLVQPAVCALNNDQYVRLPIGDERDLLDNKLQIQERSRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKSMDEAAEYYGISEKYKA

SEQ ID NO: 502 - protein, 539 aa, unidentified
  source 1..539: note = Uncultivated organism; MG119-164 effector
MNSNNTQIVTRKIVIRPTFSERKEWKKKVTEFTLKDLEEQISKYEEWIENLKSNKKIDKEEKERRKQKYTDLLTNAENSLNQFKDNEEITQKMVNRYTETLVRESMESEARRKNYILTWAYFSMIANGVPDMQLKEQKKFIREMLKPAYRVKKSSKGSIFDDVNIDNILGGYGISFNQELTNKIIDSVEKGLFEGGKDSLPSYKYDSPFTVAKSAMGFSHDYDSFEELCEHIKKKDCNLYFDFGSNSDATIARFIIDVGVNRNKDELLTTLCRVYSGEYEYCGSSIQFDKTGKKIILNLTVKMPQKNCDLNENTVVGVDLGIKIPAVCALNTNPYAREFIGSVDDYLRVRTQIQNQRRRLSIQLRNTKGGHGRKKKLKALDRYTSRERNFVKNYNHMVSSRIIKFALKYNAKYINLECLKGYDTDKKVLRNWSYYELQSMIEYKANKYGIVVRKINPCYTSQVCSECGHYEEGQRVSQEKFICGNDGCRFHNAKNGYINADFNAARNIAKSELFLDSEVTKKRIEEAAKYYNIHINDEV

SEQ ID NO: 503 - protein, 533 aa, unidentified
  source 1..533: note = Uncultivated organism; MG119-165 effector
MNKNNDRKTQCIITRKFTLIPEVSDRKEWVKRVLNYVIEECDKKIEYFEKKQSGTSQNVNGKIKEKIQVLQLKKELAKSGKLEIDKKLAVDYTYDTVRRSMKSEAARKNYILSWAFSEMKGNGVSYMEPKERKKFIEELLKPAYRKKGSKLGSLFDDTEIENILKSYGVAFSKSFTKQLRKDVANGLLDGEISLRTYKSNSPFTIAKEHISLTHNYETYEELRENIGKKDCKLYFNFGGNGIPATLRFLINTGTAKNKDELNTTLLKLYSGEYELSGSSIQLDKTGKKIILNLSMIIPMKYHELDENTVVGVDLGVAIPAVCALNNDMYKREVMGSADDFLRVRMKIQAQRRRLQSSLKYNSGGHGRKKKLKALERLSKSEIHFVETYNHMVSRRVVDFAIKNNAKYINIENLKGYNTSQYILRNWSFYQLQQYITYKAEQHGIEVRKINPCYTSQVCSVCGHWEEGQRKSQAVFECGNPDCESHSKYKNGFNADFNAARNIAKSTLFMGDGEVTEKSKQEAREYCGIYREED

SEQ ID NO: 504 - protein, 525 aa, unidentified
  source 1..525: note = Uncultivated organism; MG119-166 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKNTVVGVALGLVQPAVCALNNDQYVRLPIGDERDLLDNKLQIQERSRRLQSALRETSGGHGRKKKMQALNRIAKTENNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTNYKKGFTADFNHARNVAMSTLFMENGSFSKKSMDEAAEYYGISEKYKA

SEQ ID NO: 505 - protein, 565 aa, unidentified
  source 1..565: note = Uncultivated organism; MG119-167 effector
MKTNKDETTITITRKYAIIPTSSETKEWNKRIYAFTADKLNKKITSLTEDIEEINDKPEKKKTDEDRKHLEELNKSLSDANSTLEEITKTKEYTQKMINDYTYSLVRTAMEEEARRKNYILSWMFSEMTKHGVQYMETLKEKQKFVKDTINYAYRKKGSKAGSLFDNTEITNILNGYGTAFKQAFTGKINDAVKDGLLEGKVSLPSYKIDSPFTLEKTTMGFTHDYENYEELCEHVNDKDLKLYFDYGGNQNPTIARFKINLGHGKNKDELKATLLKLYSGEYQYCGSSIQISKNKIILNLSMKIPKKEMDLDENVVVGVDLGMEVPAMCALNNSIYEKQPIGSADDFLAIRTRLQAQRRKLQIALKSTAGGHGRKKKLKALNRLRDREKHFVESYCHFVSKQVVDFALKHRAKYINIENLTGYDTSKFILRNWSYYKLQQYITYKAARYGIIVRKINPCYTSQVCSYCGHWEPKQRKSQDTFICGSEDCISNNKKKVKYTVNADFNAARNIAMSTLFMETGEVTEKTKEKAREYYGFEEEYQKYKAKNEEAKENKEEELYDLAA

SEQ ID NO: 506 - protein, 539 aa, unidentified
  source 1..539: note = Uncultivated organism; MG119-168 effector
MDSNNTQIVTRKLVIRPTFSERKEWKKKVTEFTLKDLEEQISKYKEWIENLKSNKKIDKEEKERRKQKYTDLLTNAENSLNQFKDREEITQKMVNRYTETLVKESMESEARRKNYILTWAYFSMRANGVQHMQPKEQKKFIREMLKPAYRVKNSSKGSIFDDVEIDNILGGYGMAFNQELTNKIIDSVEKGLFEGGKDSLPSYKYDSPFTVAKSTMGFSHDYDSFEELCEHIKEKDCNLYFDFGGNSDATIARFIIDVGTNRNKDELLTTLCRVYSGEYEYCGSNIQFDKTGKKIILNLTIKIPQKNCDLNENTVVGVDLGIKIPAVCALNTNPYAREFIGSVDEYLRVRTQIQNQRRRLSIQLRNTKGGHGRKKKLKALDRYTSRERNFVKNYNHMVSSRIIKFALKYNAKYINLECLKGYDADKKALRNWSYYELQSMIEYKANKYGIVVRKINPCYTSQVCSECGHYEEGQRVSQEKFICGNDDCRFHNTKSGYINADFNAARNIAKSELFLDSEVTKKRIEEAAKYYNIPTKDEV

SEQ ID NO: 507 - protein, 538 aa, unidentified
  source 1..538: note = Uncultivated organism; MG119-169 effector
MIKKQDNEIETIIITRKLNIIPIKSENNTCIKKVYKFHIDKINKKIEALVECESKEKDKKKEEKITDLSTDEKNKLEYFLNGEELTRPMVNDYTYTLVRDAMESEARRKNYILSYAFAKMTEARVDLMDIKERNKFIGQLLNPAYRAKGSKKGSIFDETEIDNILGGYGTGFSQELTSKIKDCVKDGLLDGKVSLPSYKLDSPFTIVKDHMGFTHGYDSYEELCEHINDKNCELYFNYGRHHEPTIAKFRINVGHKGNREELLTTLRRLYSGEYQHCGSSIQFDKTGRKIILNLSMKIPKIHMELDENTVVGVDLGIAIPAMCALNNNMYKREAIGSAEDFLRIRTKIQKQRRHVQTSIKNAKGGHGRKKKLKALTRYRQYEKHFVETYNHMVSSRVVKFALSNRAKYINIENLTGYDTSKFILRNWSFYQLQQYITYKAAKYGIEVRKVNPCYTSQVCSVCGNWEEGQRVTQSKFECGNKDCKSHDFKHGFNADFNAARNIAKSTLFMKDGQVTEKSKAEARKYYSIPENIEENKED

SEQ ID NO: 508 - protein, 552 aa, unidentified
  source 1..552: note = Uncultivated organism; MG119-170 effector
MINKDKNENTVTITRKYALIPVSSDKTQWYKKVYDFTIEDLTRKIEYYKEKLKKEKDKEKKEQIKTKIKNFEEGLESIYNGGDFTQKMINDYTYNLVRTAMEEEARRKNYILSWIFSEMRLNRVDQMESLKDKFKFISDTINYAYRKQGSNKGSLFDDTEISSILKSYGIAWSQELTKEIKNLVKNGALEGKVSLTNYKLDSPFTIAKAHFSFDHDYDSFEELCEHIDDSDCKMYMNYGGDNKKDGTNPASLARFRINLGQGKNRNELKATLLKVYSGEYQYCGSSIMISKNKIILNLSMKIPKIETELDENTTVGVDLGIAVPAMCALNNDMYKRLAIGSADDFIRVRTKHQEQRRRLQKSLKNSNGGHGRKKKLKPLERMNKAEAHFVETYCHMVSKRVVEFAVKNRAKYINIENLNGYDSSEFILRNWSFYKLQQYITYKAERYGIVVRKINPCYTSQICSVCGNWEPDQRKTQAKFECANEECASHDKYKYGFNADFNAARNIAMSTLFMETGEVTEKKKEEARKYYGIEEKYQRYQASLKEKDDKVA

SEQ ID NO: 509 - protein, 568 aa, unidentified
  source 1..568: note = Uncultivated organism; MG119-171 effector
MGTELQNSTIMKLNIKNKVDRESTKMELKNTQIVTRKLVIKPTFSYRKEWKKKVSDFTIKDLEDRILKYKEWIENLKVNKKLKKDEKERRKQKYTDLLRDAEKTLEEFKSGGEITQKMVNRYTESLVKDSMESEARRKNYILTWAFYNMLANGVQYMEFPEQKKFIQEMLKPAYRKKNSSKGSIFDDVEIDNILNGYGIAFSQELTSIIVEGVKNGLFSGSKETPPRYKYDSPFTVAKSTMGFSHDYDSYEELCEHINDKDCKLYFDFGGNQNPTIARFIIDLGTNGNKCELTTTLLRVYSGEYKYCGSSIQFDKTGKKIVLNLSLEIPQKECSLNENIVVGVDLGIKVPAVCALNVNPYAREYIGSVDDFLKIRTQIQNQRRNLSKQLKNTTGGHGRTKKLKALDRYTKRERNFVKNYNHMVSSRIIKFALKNNAKYINIECLKGYDTDKKILRNWSYYELQQMITYKANKYGIVVRKINPCFTSQVCSECGNYEEGQRISQEKFMCKSEKCRFHNLKKGYINADFNAARNIAKSELFLDSEVTKKKIEEAARYYNIPYEFKNTDEV

SEQ ID NO: 510 - protein, 535 aa, unidentified
  source 1..535: note = Uncultivated organism; MG119-172 effector
MAAKDNTMVITRKIALIPVVSERKEWKKRINAFLEKDFPRKIEAKKKQIKNTSKPENKDGYKLQLAELEKQYEDFKENRIEEYTQKMVNDYTYGLVRDAMESEARRKNYILSYIYTKMVQDGVGYMPTLKDKYTWIKENINVAYRRKGSNKGSIFDDVDIDNPLGGYGTCFSQILTSKIKDLVKQGLLDGKVSIPSFKIDSPFTIAKAYFDLYHEYEDIAELKKRIYDSDCKLYCGLGNKGIPTIANFKVNIGHKGNRDELISTLLRIFTGEYEICGSTMQISKTKIILNLSIRIPKTQLELDEDTVVGVDLVVAIPAVCSLNNNKYKREYIGSSDDFIRMRTKLQHEKQQLQKALKMSKGGHGRKRKLLALERCKKKEHNFVETYCHKVSKKVVDFAVKNKAKYINVENLSGYDSSKFILRNWSFYKLQQYIEYKAAKYGIEVRKINPSFTSQVCSFCGHWEDGQRKDQATFVCGNPNCESHKLHMVNADYNASRNIAMSTLFTSDKFQFCKKTMQNAAEYYGIQLDNEDQLAA

SEQ ID NO: 511 - protein, 521 aa, unidentified
  source 1..521: note = Uncultivated organism; MG119-173 effector
MAKSSENVLTRKVQLIPEISDRKKYTKRLTKYINDSYSEKIDKKRNKKFNRKDYDSLEKFKEAKEKNESEIQNLQDALANLAAGTITKKMVDDYTYGLVREAMESESSRKNYILSHVYSQMVTSDYAHMDKEEREQFFKDIWNPCFRKKGSNLGSIFDNTDIDNILGGYGTGFSQELKGKINNAIKSGLLEGKVSLPMYKKDSPFSIESQYLKFYHAYESFEELENNIEKPNCQLFMGFGKNGNSTIANFKVNLGCGSGAKKRKDNSICQTMLKVLAGEYKICGSSIQISKGKIMLNLSIDIPIKEHKLDPNICLGVDVGYVIPAMCGLNKGKLTFRDARFGDGTDLNGRRKRIREQKKRMQVGIATNKGGHGRAKKIKALDRFREKERNFARTYNHKVSHDIVNLALKNNAMYINLENLKGITSDEKKKRELGDWSYFQLQEFITYKAKQHGIIVRKVNPYRTSQTCSCCGHWEEGQRVDRDTFICKNPECKWFGKKIHADLNAAINIAKSTDFSVKKDA

SEQ ID NO: 512 - protein, 532 aa, unidentified
  source 1..532: note = Uncultivated organism; MG119-174 effector
MANKSSKKPLIITRKYALIPIESPSPIWTKSVISFLNKDYISKIEYKKQLIEIENKKGKKKDNNKIEKWEKEIANIESKIELVDNGDFTSGIVNDYTYNLIRKCCEEQSIIKNHTMFAMGAAYKDCIIKGIDEKERTEIVTDVFNSWSRVPNSSKGSMLDKMNIDLSIGSYELAYVNALKNKFWECVKDGFAYARQTLPYYKSDCPMDIASKEMSFTHDYESFEELCEHINEKPNLYFNYGGNGKPHIFRFKINTGHGKNNDELMATLMKVYAKEYKVCGSSIQIQKSGNDKKDKIILNLSLEIPKVKRELDKNICVGVDLGIAIPAMCALNTNDYVRQSIGSKDDFLRVRTKISNQRSRLQASLKMSNGGHGRKKKMKPMDRFEDYEANWVQNYNHFVSKQVVDFAIKNKAKYINIENLEGFDANNYLLAKWSYYQLQQYISYKAKINGIEVRKINPYHTSQRCSCCGYEDKGNRPKGKKKQAYFKCLKCGKEMNADFNAARNIAMSTEWSDGKTTKEQKKKQHEEYIKKE

SEQ ID NO: 513 - protein, 525 aa, unidentified
  source 1..525: note = Uncultivated organism; MG119-175 effector
MAEKMTMITKKCVLIPERSDSSLWERRVYDFTVKDYEQRISFYESQLESKNKEVKRNAKTRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTKYKKGFTADFNHARNVAMSTLFMENGSFSKKSMDEAAEYYGISEKYKA

SEQ ID NO: 514 - protein, 525 aa, unidentified
  source 1..525: note = Uncultivated organism; MG119-176 effector
MAEKMTMITKKCVLIPERSDSSLWERRVYDFTVKDYEQRISFYESQLESKNKEVKRNAKTRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKSMDEAAEYYGISEKYKA

SEQ ID NO: 515 - protein, 579 aa, unidentified
  source 1..579: note = Uncultivated organism; MG119-177 effector
MKKNNKESNTYIKAMKIRIVPICDIEEENLRDKYYKELYQYLRDSIWAQNAALNYGLAMTRDAYVLHKNEDKIKDIYFKLAHQTPNPSMASEARLKEVYEAAPITQEYIDNKVEEFKKSNSKKKKPLTDEGVKKKTDSINNMYKKFIGLSKEDIQELIDKLDDYCAYPEEIYEKFANGFSTPAYVTQQIKDYWNTEGVMTKVIYSMSDKLRSVKNSNPLTIPPNLFYNKKNDLIGITHSYNTYLEFVEALMNVYDANLFLELPYKKGYDKMKFKLILGNPCKSHEVRISLQRIFEEYYKIRGSKIGFVRNKKTGKNTDLVLYLSVEIPKDTSIELDENTVVGVDLGLAVPAVCALNNNPYPRKYIGKKLELLKKRTQFKNRRKKLQSELRDARGGHGRRRKMKAMDRLSELEKNFADTYCHKVAKKVVMFALKNKAKYINLEMLKGYRADEKVLQNWSYYRIQMYIKILAERYGIIVRFINPCYTSQVCSVCGNWHPGNRPKGDKGQKYFDCHNDKCESHTKNKGEGYTLNADYNAARNIAMCDLFMDNKSEITEEDKRIAREKYGIPEPIKKKEKLAA

SEQ ID NO: 516 - protein, 571 aa, unidentified
  source 1..571: note = Uncultivated organism; MG119-178 effector
MVTSNTTANVRKMCKTVKIKLIPMGTKKGTAKQERERVIQYLKDSEKAQQIALNHIIGQLTAEYYACGSNLNSPEFKERRKEILNYWNPVFHDVVYLPPSSELKSAITWKLNKEFSSIIKNGLAKGERTVTNYSLHCPIPTAKNRINFEHNCSSDIEFLEKMYTKEVDVQMRWINRFRFRVDLGNLSKSKKLRKTFAQLFLGNIEKCDSSIQLCKNKEIFLNLCIMVSETLVPLNEETVVGVDLNLNHPAVCALNNDKTKMLFVGNIAFRGTRETLKAARLSKQSSLMTANGSHGRGKKLAALKSIKRRERNFAKTYNHTVARRVVNFALENNAKYINIENLQGATTQIQKDSSMEFVSSNWSYYELQQYIKYKAEAVGIEVREVNSYNTSKMCSYCGSENDEQLVSFHEFVCSNPNCESHTLFPKAMDADFNAARNIALSTEWSSGKKKFADSKKQHEDAKSEKEKSEEEKETKEKSNTAAKKAKSTDPYDFETLKANGILSARTANALTTAGYKNVSDLHGKSVKDIKKIRGFGDSAINQLFKYLNSLDFVSVPANPSEKLSVTDAPRG

SEQ ID NO: 517 - protein, 525 aa, unidentified
  source 1..525: note = Uncultivated organism; MG119-179 effector
MSEKMTMITKKCVLIPERSDSSLWERRVYDFTVKDYEQRISFYESQLKSKNKEVKRNAKTRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTKYKKGFTADFNHARNVAMSTLFMENGSFSKKTMDRAAEYYGISEKYKA

SEQ ID NO: 518 - protein, 525 aa, unidentified
  source 1..525: note = Uncultivated organism; MG119-180 effector
MAEKMTMITKKCVLIPERSDSLLWERRVYDFTVKDYEQRISFYESQLESKNKEVKRNAKIRLEILNMELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNQTLHIPKEEKDELDKNTVVGVALGLVQPAVCALNNDQYVRLPIGDERDLLDNKLQIQERSRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKTMDRAAEYYGISEKYKA

SEQ ID NO: 519 - protein, 525 aa, unidentified
  source 1..525: note = Uncultivated organism; MG119-181 effector
MAEKMTIITKKCVLIPERSDSSLWERRVYDFTVKDYEQRISFYESQLKSKNKEVKRNAKTRLEILNKELDDFLDNPETTQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKNTVVGVALGLVQPAVCALNNDQYVRLPIGDERDLLDNKLQIQERSRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKSMDEAAEYYGISEKYKA

SEQ ID NO: 520 - protein, 525 aa, unidentified
  source 1..525: note = Uncultivated organism; MG119-182 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKNHTNYKKGFTADFNHARNVAMSTLFMENGSFSKKSMDEAAEYYGISEKYKA

SEQ ID NO: 521 - protein, 549 aa, unidentified
  source 1..549: note = Uncultivated organism; MG119-183 effector
MINKDKNENTVTITRKYALIPVSSDKTQWYKKVYDFTIEDLTRKIEYYKEKLKKEKDKEKKEQIKTKIKNFEEGLESIYNGGDFTQKMINDYTYNLVRTAMEEEARRKNYILSWIFSEMRLNRVDQMESLKDKFKFISDTLNYAYRKKGSNKGSLFDDTEINSILNAYGIAWNQELTKEIKDLVKNGALEGKVSLTNYKLDSPFTIAKAHFSFDHDYDSFEELCEHIDDSDCKMYMNYGGDNKKDGTNPASLARFRINLGQGKNRNELKSTLLKVYSGEYQYCGSSIQISKNKIILNLSMKIPKIETELDENTVCGVDLGIAVPAMCALNNDMYKRLAIGSADDFLRVRTKHQEQRRRLQKSLKNSNGGHGRKKKLKPLERMNKAEAHFVETYCHMVSKRVVEFAVKNRAKYINIENLNGYDSSEFILRNWSFYKLQQYITYKAARYGIVVRKINPCYTSQICSVCGNWEPDQRKTQAKFECANEECASYDKYKYGFNADFNAARNIAMSTLFMETGEVTEKKKEEARKYYGIEEKYQASLKEKDDKVA

SEQ ID NO: 522 - protein, 452 aa, unidentified
  source 1..452: note = Uncultivated organism; MG119-184 effector
MIITKKLKVIPDGDKEEVKRVYKYIRDAQYNQYRYMNRLMSNVCMIYYKYDMDITNPNLKEEMKELYRVSNAAWIKEDNPIGSGMQATATSRVKQDFSTALKNGLAKGERRLPQYKRDFPFLMNGTAIKPFIKNVTYTDTNSGEDKIRTEYHLKWVNKISFRIWLPTNAYKDLNIQTIIAALNAVIENKEGYKICGSSIRVEDKDIFIFLTLDVPSKKTSYTPVTGRTMGVALGYDSPLVCCITENDKDSKFFAVGENLDFVENRKKRHEKYRQIQSATKECHGGNGRKRKMERLNAAKSNESNYRKDINHKLSKEVIDLATKNKVANIIIESTTADELKTVPDMLRNWSYYDLVAKITYKGNMYGILTEEMKKTEKKSTKKIKRADCCPACGEVFASGNANALTPEDYWNTSTEFVCPSCGTVEEYGKVRAFSSACKAMLPEEKVKNNKTE

SEQ ID NO: 523 - protein, 358 aa, unidentified
  source 1..358: note = Uncultivated organism; MG119-185 effector
MLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQILKNKIVLNLSLQIPKEEKDELDKDTVVGVALGLVQPAVCALNNDQYVRLPIGDERDLLDNKLQIQERRRRLQSALRETSGGHGRKKKMQALNRIAKTENNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTNYKKGFTADFNHARNVAMSTLFMENGSFSKKTMDRAAEYYGISEKYKA

SEQ ID NO: 524 - protein, 448 aa, unidentified
  source 1..448: note = Uncultivated organism; MG119-186 effector
MTNTYIIARKIQLIPVGKKEEVNRVYTYIKDGIKSQNMAMNQYMSALYTSTIMEMTKDDRKELSRLFGRISTSKLGSAYDKTIEFAKGLPMGGAIQQKVKKDFDNAMKKGLKYGKLSLPSYRDSNPLLVHVDYVRLRSTNPHLDNGLYHNYSSHEEFLNHLYNEKLELFIKFANKITFKLVLGNPRKSAEIRSVFKNIFEDYYHIKGSSIELDGTKIILNMSIEIPKKKVVLDENVVVGVDLGIAIPAVCALNTNDYVRKSIGSRDDFLRVRTQIKSQRDRLKRKLKYTRGGHGRAKKMQAMDRFTDYEKNWVQTYNHKVSKDVVDFALKNKAKYINLECLDGFSKDDKKKNYLLANWSYYQLQQYIKYKAEKYGIVVRFVNPAYTSQTCNCCGNLEEGQRVKQSEFICKNPECKNFGKKINADFNAARNISMSTDFVEKEEEKERIT

SEQ ID NO: 525 - protein, 474 aa, unidentified
  source 1..474: note = Uncultivated organism; MG119-187 effector
MANKYIISRKITLIPAAPLNNQWQKRFDKYVNLSFERVQEQIIEAQAELDDEETKKTRKDSLSKWIDSLYKKADMYSDYISEGEITKALLNDYVYTFVRNACKAESRHKNLVISKVIDTMRINGFYDLKSDEARELYRSTFNAAVRLNNGSFSDIINNPCGGYGVSWSNALNASLKREIINGCISCRNGQVKNFKEMSPFSIAKSAMGFSHGYDSDEIMLANMDEKNAPLYFDFGGNGTPSLLQFRMNLGANPKNKNELVSTIKRVYTGEYQYCGSSIQISGNKIILNLTLEIPKPADMLESLSKNTVLGLSLGTNTAIIGALNDSKSYFTFGTGHELEYYRRYIQESYRNLAKAVKYTNGGHGREKKLKKLEVFKVRERNFVKSFNHKASRTAVDFAVKHNAGLIRMPDLSEFSSKVNDDESKYLLRNWSYYQLQQMIEDKAARVGITIEYVPIPQVEDGVADIDIARKIANN

SEQ ID NO: 526 - protein, 433 aa, unidentified
  source 1..433: note = Uncultivated organism; MG119-188 effector
MILTRKIQLLIVGNNDEVNRVFNYIRKGMISQNKAMNQYMSALYLAELNKVSKEDRKELNQLYSRISNSKKGSAYSQDIVFPKGLSVASDLSMKVKQDFKQSCKNGLMYGKVSLPTYRSNNPLLIHVDYVRLRSSNPHRDSGLYHNYKNHAEFLEHLDNKDLEVFIKFANNITFKLIFGNVKKSASLRHEIQMVFEEYYKVCSSSIEIDGRKIILNLSMDVPEQKIELDENVIVGVDVGIAIPAVCGLNTNDYSRKYIGSVDDFMRVKTKIQHQKSRLQTNLKMAKGGHGKKRKLKAMDKFTDYEKNWVQSYNHYVSKQIIDFALKNKAKYINIEDLSGITKGKNVNKFLKGWSYYQLQSFIIYKANKYGIEVRKIDPHYTSQTCSCCGYVDEKNRPKNEKGQSYFRCLKCGHEENADFNAAKNIAKSVNFVK

SEQ ID NO: 527 - protein, 470 aa, unidentified
  source 1..470: note = Uncultivated organism; MG119-189 effector
MIMVKKIAIIPAASDNAMWLKKFNEYVEFSRRDAEEKYKAVSEKKNLKDKDNILKNLRNKMDRADKFLADGEITKPLLNDYVFNFCRDASREEAKHKNYVISKVIDTLRIAGYEDLSSDEAKKIYKDTFNAALRTKNSTSAAFINNPLKGYGLSWSQTLNSSLKKDIFNGAISCKNGQARNFKPDSPFTVAKSMMGFSHTYDSDGEMYAHISDTNANIYFDLGAKGKPTILRFKLNLGANPKNKNELISVIKKVYSGEYEYCASSIKIEGAKILLYLCLAIPEDKEELKSGNKVGVSLGLNTAVIAVTNANKKDVLKFGDEHTLEYYRVYIQNRLWELKKDLKYINGGHGRKEKLEKLNIYKDRERNFVKDFNHKASFAIIKFALKYQASVIVMPDLTEFAQRLTKEIKEARKNHETYSHKYMLRNWSYYELTSMVEYKAARKGITVIYKTLSEDIDGVADIDKARMMLE

SEQ ID NO: 528 - protein, 458 aa, unidentified
  source 1..458: note = Uncultivated organism; MG119-190 effector
MEKNTQIITRKYNIVPCKADTKQWYEKCYEYTISDINAKIDRAEYVLKKKDGIENRDRLQEKIGQYRKMLDEMEKHKEDFDFTNQMIDTYTKDLVREAMESEARRRNYILSWAFATMTENGLQHETDFKARNKFISEMLKPAYRVKGSNKGSLFDDTEIENILGGYGTSFNQELTRKIKDSVNKGLLEGRVSLPSYKMDSPCSIAKAHMGFSHDYSSYEELMAHIEDRELKLYFDFGSPDKDAGMAKPTIARFRFNLGGCRNRQELLTTLLRVYSGEYQYCGSSIQFNKQGNKVMLNLSMEIPVRENILDPDTVVDIDLGVDIPAICTVENSGSTLDIGSKEDLLRIRTQLQIQRGNTQRNLVIAKGGHGRKKKLRKLETLKAREANFANTYLHMVSRKAVDFAVENHAGCIRLKNIEEYDSQDYLLKNMSYYKLCQQIEYKAAIQGISVVYAEKERK

SEQ ID NO: 529 - protein, 390 aa, unidentified
  source 1..390: note = Uncultivated organism; MG119-191 effector
MSEKRMTITKKMQLLVDGDKDEVKRVYDFIRNGQYAQYRAMNRYMGEIASIYYKHNMDFKSDGFQTEYKEIRTNKNEIFQDLEFSTGVDTISQARRTVESDFMASVKNGLAKGERTITNYKREYPLLTYGRSLKFYHDYADDYEMYDNLDNKDFLIYLGWVNKIRFKVRFGNPYKSHELRTSVARICDGTYKIQGSKITIIGKKIFLFASVSIPILEKEMLEDNVCGVKFGKCVTATCVTNIDKKKYVECGNADVFLMQRAKMKAMRERTMAQLKYSKGGHGRKKKLKPLERFRKREHNFAKTYNHTISKEIVNFAFDNRCSKIVLEDLSIYENDDEFVTKNWSYFDLEEIIKYKAKRYGITVETKKLDIEKVKDENWSNVDLAFELTLD

SEQ ID NO: 530 - protein, 357 aa, unidentified
  source 1..357: note = Uncultivated organism; MG119-192 effector
MAKKQNKNTMQINRKITLIPVASDNDSWKKKMNTYLEKFYFDKKIKAKERQIKNTSKPERKDEYKQQLAEYKKQQEQFLNGELEDYTKQMVMSYTYDLVRDCMESEARQKNFIMSYMFSEMIREKVCYLKNKKEKEKWVNDNINVAFRVKGSPKGSIFDDVEIYSPLRALSIQGYTQELKGRVKKFATDGGLDGKLAVDNYKLDSPFHMSKQGFDIVHEYEDLTELKKNIGKSSCEIYVNLGNGGVPTFARFQLDFGHKGNREELISTITKVFTGEYKVCGSSIQINKKNKIILNLGLEIPKRLTELDENTVVGVDLGLAVPAVCSLNNNQYKKEYIGDGEAFVKQRGKIQKEKQRL

SEQ ID NO: 531 - protein, 419 aa, unidentified
  source 1..419: note = Uncultivated organism; MG119-193 effector
MANVLTRKIQLVPVGDKEEINRVYNYIREGQYSQYLGLNRLMSELVVTFYKNDRKLDEKFNEERKNILSNSNTILNDILFPKGCDTKSHIVQKVKSDFSKALKNGLARGEVAVTNYKRTNPLLVRGRDLKFSYDYATLNEFEQNLLNTDLKVYINFVDKIKFKIVFGNPHKSQALRLEIREILLDNYKVKGSSLQIDGKKIILNLSIAVPTKKIELDKKVTVGVDLGLAIPAVCALNTNDYIRKSIGSADDFLRQRTKIQKQKKAIQKNLVQVKGEHGRKKKLNHLEKVRKNEEHWVRTYNHFVSKQVVDFAIKNKAKYINVEDLTGFDNSKFILRNWSYYQLQEFITYKANNVGIQVRKINPYHTSQTCSCCGHWEEGQRISQSKFICKNSNCKNYGKEINADFNAARNIAQCSDFIK

SEQ ID NO: 532 - protein, 419 aa, unidentified
  source 1..419: note = Uncultivated organism; MG119-194 effector
MANVLTRKIQLVPVGDKEEINRVYNYIREGQYSQYLGLNRLMSELVVTFYKNDRKLDEKFNEERKNILSNSNTILNDILFPKGCDTKSHIVQKVKSDFSKALKNGLARGEVAVTNYKRTSPLLVRGRDLKFSYDYATLNEFEQNLLNTDLKVYINFVDKIKFKIVFGNPHKSQALRLEIREILLDNYKVKGSSLQIDGKKIILNLSIAVPTKKIELDKKVTVGVDLGLAIPAVCALNTNDYIRKSIGSADDFLRQRTKIQKQKKAIQKNLVQVKGGHGRKKKLNHLEKVRKNEEHWVRTYNHFVSKQVVDFAIKNKAKYINVEDLTGFDNSKFILRNWSYYQLQEFITYKANNVGIQVRKINPYHTSQTCSCCGHWEEGQRISQSKFICKNSNCKNYGKEINADFNAARNIAQCSDFIK

SEQ ID NO: 533 - protein, 460 aa, unidentified
  source 1..460: note = Uncultivated organism; MG119-195 effector
MKTEETIDWIDSCIVCAGRQERRKEGMLPGMFGSLILTRKIQLIPCTEGMTTDEAKVEVNRIYQLLRDGIYAQNKAYNIFISRMYTATLLGASKEELKEIRLKGERTPKESDSNYSLYDFDKIKFIKGLPQASALGEQALKALKKQQKDGLYKGKVSLACRKLDAPMWIKTKFSFFHKYNDYQEFLDHLYCDDLKIYMKFVQGIVFETVLGNPHKSETIRAEFQQIFEGHYKLCSSSLQIKDKKIMLNLAIDIPEKEIELNDETVVGVDIGIAIPAVCALNSREYIHKSIGSAEELLRIRTQLQSQKRRLQKNLKNTTGGHGRSHKLAPLDKLAKRERNFVQTYNHMISKTVVEFAVKNKAKYINLEDLSNYKDNGSEFILRNWSYFELQSQIEYKAKMHGIIVRKINPYHTSQICSKCGHWEEGQRISQSKFKCKSCGYESNADFNAARNIALSTDFIE

SEQ ID NO: 534 - protein, 457 aa, unidentified
  source 1..457: note = Uncultivated organism; MG119-196 effector
MTICRKIKLFPVGDKDEINRVYDFIRNGQYAQYQACNLLMGQLMSEYYKYNRDIKNEEFKARQKEIMTNSNIILKDIDFATGVDTPSAVTQKVKQDFSTALKNGLAKGERTVTNYKRTNPLITRGRNLTFYHEYETYHDFLDKINDSDLAVYVKWVNKIVFKVVFGNPHRSLELRSVIQNILEENYKVQGSSIEIDGKSIILNLSISIPKQLRELDENIVVGVDLGIAVPAMCALNNNLYERLAIGNADDFLRIRTKMQAQRKRLQKSLRNTSGGHGRAKKLKALERLQKAEVHFVETYCHMISKRVVDFALKHNAKYINIENLTGYDTSDFILRNWSYYKLQDYITYKAAKYGIEVRKINPCYTSQICSVCGNWEFGQRKSQSVFECANENCDSHKKYEKTGFNADFNAARNIAMSTLWMGSGQVTEKSKQEAREYYDISEKYEQSKNGSENNKVA

SEQ ID NO: 535 - protein, 426 aa, unidentified
  source 1..426: note = Uncultivated organism; MG119-197 effector
MILTRKIQLIPCTEGMTTDETKVEVNRIYHLLRDGIYAQNKAYNIFISRMYTATILGASKEELKEIRLKGERTPKESDSDYSLYDFDKIKFIKGLPQASALGEQALKALKKQQKDGLYKGKVSLACRKLDAPMWIKTKFSFFHKYNDYQEFLDHLYCDDLKIYMKFVQGIVFETVLGNPHKSETIRAEFQQIFEGHYKLCSSSLQIKDKKIMLNLAIDIPEKEIELNDETVVGVDIGIAIPAVCALNSREYIHKSIGSAEELLRIRTQLQSQKRRLQKNLKNTTGGHGRSHKLAPLDKLAKRERNFVQTYNHMISKTVVEFAVKNKAKYINLEDLSNYKDNGSEFILRNWSYFELQSQIEYKAKMHGIIVRKINPYHTSQICSKCGHWEEGQRISQSKFKCKSCGYESNADFNAARNIALSTDFIE

SEQ ID NO: 536 - protein, 412 aa, unidentified
  source 1..412: note = Uncultivated organism; MG119-198 effector
MGTFCKKIRLYPIGDQEEVNRVYEYIRNGMYVQYTLMNKLMSQIMTLYYSDEVDRDVKSDAFKNGRREILRISNPVFHDEPMPKGLGMQSTVTRKVSQDFSTALKNGLASGDRSAPYYRRSCGLMVPGVRFQFYMSDDTYCIKFCNGIHFKVITGRGKRDYYLTDLLDALSSGNEDYHIGTSQLTIDGKYIYLILTVSKEDVEVSYEPDKDRVMGIAFGYESPTVCCIRNLTKPQVSYIEIGDSTPLVKDRMRIQENMRMLTTGLKNCTGGRGRKHKLQALDRYTKRERNFVRTYNHKLSKDIVSAALDAQVSDIVMEKVEKQDVSAVMLRNWSWYELQSMVSYKAAKYGITVSVDDRKIGLVCSQCGCVQSEQADSEFNTSGTFFCTECGHRMPAGQNKALILVSDTKEKA

SEQ ID NO: 537 - protein, 419 aa, unidentified
  source 1..419: note = Uncultivated organism; MG119-199 effector
MANVLTRKIQLVPVGDKEEINRVYNYIREGQYSQYLGLNRLMSELVVTFYKNDRKLDEKFNEERKNILSNSNTILNDILFPKGCDTKSHIAQKVKSDFSKALKNGLARGEVAVTNYKRTSPLLVRGRDLKFSYDYATLNEFEQNLLNTDLKVYINFVDKIKFKIVFGNPHKSQALRLEIREILLDNYKVKGSSLQIDGKKIILNLSIAVPTKKIELDKKVTVGVDLGLAIPAVCALNTNDYIRKSIGSADDFLRQRTKIQKQKKAIQKNLVQVKGGHGRKKKLNHLEKVRKNEEHWVRTYNHFVSKQVVDFAIKNKAKYINVEDLTGFDNSKFILRNWSYYQLQEFITYKANNVGIQVRKINPYHTSQTCSCCGHWEEGQRISQSKFICKNSNCKNYGKEINADFNAARNIAQCSDFIK

SEQ ID NO: 538 - protein, 474 aa, unidentified
  source 1..474: note = Uncultivated organism; MG119-200 effector
MENKMIMVKKIAIIPAASDNAMWLKKFNEYVEFSRRDAEEKYKAVSEKKNLKDKDNILKNLRNKMDRADKFLADGEITKPLLNDYVFNFCRDASREEAKHKNYVISKVIDTLRIAGYEDLSSDEAKKIYKDTFNAALRTKNSTSAAFINNPLKGYGLSWSQTLNSSLKKDIFNGAISCKNGQARNFKPDSPFTVAKSMMGFSHTYDSDGEMYAHISDTNANIYFDLGAKGKPTILRFKLNLGANPKNKNELISVIKKVYSGEYEYCASSIKIEGAKILLYLCLAIPEDKEELKSENKVGVSLGLNTAVIAVTNANKKDVLKFGDEHTLEYYRVYIQNRLWRLKKDLKYINGGHGRKEKLEKLNIYKDRERNFVKDFNHKASFAIIKFALKYQASVIVMPDLTEFAQRLTKEIKEARKNHETYSHKYMLRNWSYYELTSMVEYKAARKGITVIYKPLSESIDGVADIDKARMMLE

SEQ ID NO: 539 - protein, 471 aa, unidentified
  source 1..471: note = Uncultivated organism; MG119-201 effector
MILTRKIQLLPLGDKEELNRIYTYIRDGMFNQNKGMNQFISALYVSFINEFAKEDRKELVRLYGRNPESSKGSAYSADMSFAKGLGSASSLTRKCQQSFDKAVKDGLLYGRVSLPTFRMDNPLLVPGRFIDVKGNRIKNDGHTETRGIYHLYASHEEFLDHLHNNDLEIYLDFVNGIHFKFVLGNPHKSRFLREEIKQIFEGYYKICDSSIGIDGKKIMLNLCMDVPKKEIVLDENKVIGVDMGLAVPAVCVCYNRSTNKYEGTPLYIGEIDELLTVRTRLQAERRRLQKALADCTSGGHGRKKKLAKLEDLKKRERHFVQTYNHMVSRRIVKFAEEHGAKYINVEDLSDLDSKLKGKLKDKFDEESGQFVLRNFSYFERQNYTEYKAARYEMVVRKINPDKTSQTCSCCGNYEPGQRISQSAFKCKKCGYEENADVNAAKNIAFSENFVEKVKTPKKTKKNEQLDGIEND

SEQ ID NO: 540 - protein, 459 aa, unidentified
  source 1..459: note = Uncultivated organism; MG119-202 effector
MEIKANRMIITKKLKVIPDGDKEEVKRVYKYIRDAQYNQYRYMNRLMSNVCMIYYKYDMDITNPNLKEEMKELYRVSNAAWIKEDNPIGSGMQATATSRVKQDFSTALKNGLAKGERRLPQYKRDFPFLMNGTAIKPFIKNVTYTDTNSGEDKIRTEYHLKWVNKISFRIWLPTNAYKDLNIQTIIAALNAVIENKEGYKICGSSIRVEDKDIFIFLTLDVPSKKTSYTPVTGRTMGVALGYDSPLVCCITENDKDSKFFAVGENLDFVENRKKRHEKYRQIQSATKECHGGNGRKRKMECLNAAKSNESNYRKDINHKLSKEVIDLATKNKVANIIIESTTADELKTVPDMLRNWSYYDLVAKITYKGNMYGILTEEMKKTEKKSTKKIKRADCCPACGEVFASGNANALTPEDYWNTSTEFVCPSCGTVEEYGKVRAFSSACKAMLPEEKVKNNKTE

SEQ ID NO: 541 - protein, 401 aa, unidentified
  source 1..401: note = Uncultivated organism; MG119-203 effector
MYLVRKIRLIVIGENKEDVNEKYKFIRDAQYAQYRGLNYMMGQVGALYYSCDRNISSDEFKTQYMEIFRASNQVVDGIEWGTGIDTRSLVGTRIKQDFSTALKRGLAKGEISLPNYKRSYPLLTKGRDIRFYTDYTEGELVHDMVLEPDFNLYIKWVHKIHFKVVLGNPYRSRFLREELWKVLCGEYKIAGSSLGFDKNGKLILNLSMDIPVNKEKKENMNNVLGVRLAYAKPVVAVVNSTGRCFMIGDGERLIGYRKKMRNNIRRVQQDVTLAAGGSGRAKKLAALNRFKDREANFAKTYNHQISRRIVEIAKNNGCSTIVLEDIGNIYDVDNIVLSNWGYYQLHQQIEYKAQMQDINVVKSQLVAEKQMQTMLETIELDKRDEFIAKSLSQIDIKQKKK

SEQ ID NO: 542 - protein, 459 aa, unidentified
  source 1..459: note = Uncultivated organism; MG119-204 effector
MEIKANRMIITKKLKVIPDGDKEEVKRVYKYIRDAQYNQYRYMNRLMSNVCMIYYKYDMDITNPNLKEEMKELYRVSNAAWIKEDNPIGSGMQATATSRVKQDFSTALKNGLAKGERRLPQYKRDFPFLMNGAAIKPFIKNVTYTDTNSGEDKIRTEYHLKWVNKISFRIWLPTNAYKDLNIQTIIAALNAVIENKEGYKICGSSIRVEDKDIFIFLTLDVPSKKTSYTPVTGRTMGVALGYDSPLVCCITENDKDSKFFAVGENLDFVENRKKRHEKYRQIQSATKECHGGNGRKRKMERLNAAKSNESNYRKDINHKLSKEVIDLATKNKVANIIIESTTADELKTVPDMLRNWSYYDLVAKITYKGNMYGILTEEMKKTEKKSTKKIKRADCCPACGEVFASGNANALTPEDYWNTSTEFVCPSCGTVEEYGKVRAFSSACKAMLPEEKVKNNKTE

SEQ ID NO: 543 - protein, 426 aa, unidentified
  source 1..426: note = Uncultivated organism; MG119-205 effector
MILTRKIQLIPCTEGMTTDEAKVEVNRIYQLLRDGIYAQNKAYNIFISRMYTAILLGASKEELKEIRLKGERTPKESDSDYSLYDFDKIKFIKGLPQASALGEQALKALKKQRKDGLYKGKVSLACRKLDAPMWIKTKFSFFHKYNDYQEFLDHLYCDDLKIYMKFVQGIVFEVVLGNPHKSETIRTEFQQIFEGHYKLCSSSLQIKDKKIMLNLAIDIPEKGIELNDETVVGVDIGIAIPAVCALNNREYVHKSIGSAEELLRIRTQLQSQKRRLQKNLKNTTGGHGRSHKLAPLDKLAKRERNFVQTYNHMISKTVVEFAVKNKAKYINLEDLSNYKDNGSEFILRNWSYFELQSQIEYKAKMHGIIVRKINPYHTSQICSKCGHWEEGQRISQSKFKCKSCGYESNADFNAARNIALSTDFIE

SEQ ID NO: 544 - protein, 426 aa, unidentified
  source 1..426: note = Uncultivated organism; MG119-206 effector
MILTRKIQLIPCTEGMTTDEAKVEVNRIYHLLRDGIYAQNKAYNIFISRMYTATILGASKEELKEIRLKGERTPKESDSDYSLYDFDKIKFIKGLPQASALGEQALKALKKQQKDGLYKGKVSLACRKLDAPMWIKTKFSFFHKYNDYQEFLDHLYCDDLKIYMKFVQGIVFETVLGNPHKSETIRAEFQQIFEGHYKLCSSSLQIKDKKIMLNLAIDIPEKEIELNDETVVGVDIGIAIPAVCALNSREYIHKSIGSAEELLRIRTQLQSQKRRLQKNLKNTTGGHGRSHKLAPLDKLAKRERNFVQTYNHMISKTVVEFAVKNKAKYINLEDLSNYKDNGSEFILRNWSYFELQSQIEYKAKMHGIIVRKINPYHTSQICSKCGHWEEGQRISQSKFKCKSCGYESNADFNAARNIALSTDFIE

SEQ ID NO: 545 - protein, 452 aa, unidentified
  source 1..452: note = Uncultivated organism; MG119-207 effector
MAKDRVTICKTIKLYPVGDKSEVDRVYKFIRDGQYAQYRASNRLAGELYSEYYRFNMDIKNEEFKTRQKEIMRNSNPLFDDIEFAIGVDTPSAVTQKVKSDFSIALKNGLARGERNTTNYKRTNPLITRGRSLKFVHEYENYQEFLDHINSSDFAVFIKWVNKIKFKIVFGNPHRSQELRGIVKNIFEDNYKVQGSSIQVDGNEIKLNLSLSIPKEIKELDENTVVGVDLGIAIPAMCALNNNLYERESIGKVDDFTRVRQKYQAQVKRLQKSHKSATGSHGRSKKMRALNRVKKSEAHFVETYCHYVSKKVVDFALKHNAKYINMENLKGYDTSRFILRNWSYYKLQQYITYKADRYGIVVRKINPCYTSQVCSVCGHWEPDQRKTQASFECANPGCESHKKYKYGFNADFNAARNIAMSTLFMQDEEVTEKKKEEACEYYGIKKENSEAV

SEQ ID NO: 546 - protein, 462 aa, unidentified
  source 1..462: note = Uncultivated organism; MG119-208 effector
MGNDRMTICRKIKLFPVGDKEEINRVYNFIRNGQYAQYQACNLLMGQLMSEYYKYNRDIKNEEFKARQKEIMTNSNIILKDIDFATGVDTPSAVTQKVKQDFNTALKNGLARGERTVTNYKRTNPLITRGRNLTFYHEYETYQDFLDKINDSDLTVYVKWVNKIVFKVVFGNPHRSLELRSVIQNILEENYKVQGSSIEIDGKSIILNLSISIPKQIRELDENTVVGVDLGVAVPAMCALNNNLYERLAIGDADDFLRIRTKMQAQRRRLQKLLRNTSGGHGRTKKLKALDRLQKSEAHFVETYCHMISKRVVDFALKHNAKYINIENLTGYNTSDFILRNWSYYKLQDYITYKAAKYGIEVRKINPCYTSQICSVCGNWEFGQRKSQSVFECANEDCDSHKKYEKTGFNADFNAARNIAMSTLWMENGQVTEKSKQEAREYYGISEKYEQSKNDSENNKVA

SEQ ID NO: 547 - protein, 474 aa, unidentified
  source 1..474: note = Uncultivated organism; MG119-209 effector
MENKMIMVKKIAIIPAASDNAMWLKKFNKYVEFSRRDAEEKYKAVSEKKNLKDKDNILKNLRNKMDRADKFLADGEITKPLLNDYVFNFCRDASREEAKHKNYVISKVIDTLRIAGYEDLSSDEAKKIYKDTFNAALRTKNSTSAAFINNPLKGYGLSWSQTLNSSLKKDIFNGAISCKNGQARNFKPDSPFTVAKSMMGFSHTYDSDGEMYAHISDTNANIYFDLGAKGKPTILRFKLNLGANPKNKNELISVIKKVYSGEYEYCASSIKIEGAKILLYLCLAIPEDKEELKSENKVGVSLGLNTAVIAVTNANKKDVLKFGDEHTLEYYRVYIQNRLWRLKKDLKYINGGHGRKEKLEKLNIYKDRERNFVKDFNHKASFAIIKFALKYQASVIVMPDLTEFAQRLTKEIKEARKNHETYSHKYMLRNWSYYELTSMVEYKAARKGITVIYKPLSESIDGVADIDKARMMLE

SEQ ID NO: 548 - protein, 466 aa, unidentified
  source 1..466: note = Uncultivated organism; MG119-210 effector
MILKSHVNGLQIPINTRAANILRENTRMSKRTTMIQRVSLIPIGEKEQTDKAYKYISDSIYAFYKTNNQIMGEIYSAFYNNGCNLNLEEYKAEHSELFSYDNPAISKIEYPGGIDILELSIKKVESDFKKALLNGLAQGKITAKNYKRTEPLPVSKETLNFDIKFENLKNGTPNIRSLSATLNWVNGISFEVYFEKSGYGFNTQNSFYKIHTGEYTIEDSKIIIEKNKRGIRGINLYLSLNVPGKVHTLNEDTVIGVNLGLSRTIVCCLNTDNYKRLYLEPDLLIKSTEQRIYSAEKDVKKLSLSTRTEHKRQAKEQFVNRLKHCKESLIKRINDKATTNILLFAQKNNAKYINIENMAWYGAPQKNSLLVRNGNYADFEEQLIRKSHKFGMIVRKINPYSTMHTCSFCGCQEDGQLISDTRFLCKNPDCVSHTFKGKGEANKKYIEPDFNAARNIAKSTEFKKSH

SEQ ID NO: 549 - protein, 466 aa, unidentified
  source 1..466: note = Uncultivated organism; MG119-211 effector
MILKSHVNGLQIPINTRAANILRENTRMSKRTTMIQRVSLIPIGEKEQTDKAYKYISDSIYAFYKTNNQIMGEIYSAFYNNGCNLNLEEYKAEHSELFSYDNPAISKIEYPGGIDILELSIKKVESDFKKALLNGLAQGKITAKNYKRTEPLPVSKETLNFDIKFENLKNGSPNIRSLSATLNWVNGISFEVYFEKSGYGFNTQNSFYKIHTGEYTIEDSKIIIEKNKRGIRGINLYLSLNVPGKVHTLNEDTVIGVNLGLSRTIVCCLNTDNYKRLYLEPDLLIKSTEQRIYSAEKDVKKLSLSTRTEHKRQAKEQFVNRLKHCKESLIKRINDKATTNILLFAQKNNAKYINIENMAWYGAPQKNSLLVRNGNYADFEEQLIRKSHKFGMIVRKINPYSTMHTCSFCGCQEDGQLISDTRFLCKNPDCVSHTFKGKGEANKKYIEPDFNAARNIAKSTEFKKSH

SEQ ID NO: 550 - protein, 442 aa, unidentified
  source 1..442: note = Uncultivated organism; MG119-212 effector
MPNITRKYQLKVIGDKEEIDRVYKYIREGTEAQNKALNEAMSALYAANLLDMSKDDKKELSKLFSRVTNGKNESGFTNDICFATGLGTTANIKQKVKQDFNNACKKGLMYGRVSLPSYKTDNPLLVSKSYVQLLSEGDKNFGIYSAYETPMDLVDALEKETNPEVYLKFANNILFKFVFGNPWKGREQRKVFERIFSGEYKICGSSIGIDGKKIILNLCMDIPKQEHNLDENIVVGVDLGLAIPAMCALNNDDYKRLSIGSIDDLLRVRIQLQNERKRIQGNLKNSKGGHGRQKKLKALENLKDRERNFVQTYNHMVSKRVVDFAVKNNARYINIEDLSGFGKTRYGKSKSDDEKVLRNWSYYELQNYITYKAQLHGITVKKVRAEYTSQTCSYCGSKGIRKEQKKFVCVNPDCKCHKIYDGYINADFNAARNIAMSNDFVE

SEQ ID NO: 551 - protein, 474 aa, unidentified
  source 1..474: note = Uncultivated organism; MG119-213 effector
MAITTKKIQLKIVGDNNEKNRVYTYLRDGIYNQHLVLNTYMSQVGTLYYSVNGDIKSVEWKEKYNEIFRNTNEQVKVIPYPKGLNMQANAGRKVQADFSTALKNGLASGERQLPYYKKDVPLIVPSRFLHFYSEEETYEKDGIKDTRNAYYIKFVNGIVFKVVIGPSAIKDFYFLPLLDGICNNPLVYDVRGSSIQIAKGNKIILNLTVAVNKHTEDYTPIKGRTMGVAFGYNRSLTVALSDDDNTYYIGDKVFGKNINEEIISKRIELQDYRTRLQKSVRGAKGGHGRTRKIEALDKFRRSEKNMVKHYNHILSKEVVNFAKNHNVEAIIFEDVDKDDLKDNPVMLRNWSYYELCNFIKYKAKSICKNEDNEIKVLMANANGIVEEKKKKKKKDKNASKEKPIEVKTEKNVKEDDTDKIRTKCHICGFAIRDKDRQKVSKVDEWISEQYFTCPNCDNLIEYSYNKAKNMSLLG

SEQ ID NO: 552 - protein, 456 aa, unidentified
  source 1..456: note = Uncultivated organism; MG119-214 effector
MPNLTRKYELQVVGDKEEINRVYTWLRQGIEAQNKAMNEYMSHLFMAKSCEMSLDDRKELNNLFQRVTNSKKGSGYTKDIEFPVGLPIAASVSRKVKQDFDNACKKGLMYGRISLPTYRSNNPLIIHVRFVALRGTKKVDNGIYHNYNSPADLFSALENDTKPEVFIKFCNNITFKFRFGNPYKAKEQREVFKKIFTEEYKVGGSTIEIKGTKIIINLTLEVPQIKKCSDENITVGVDLGQAIPAMCALNNDYYKRLAIGNVDDFLRIRTQMQEERRRLQCHLRNANGGHGRKKKLRALERLKQREKNFVQTYSHMVSRKVVDFAVKHNAKYINIEDLSGFGRDKYGHVKDDEQAKMVLRNWSYYDLQEKIKYKANIYGIEVRKIDPKFTSQICSVCGEKGIRQTQKEFICINPECSCHKMYQNQKYINADFNAARNIAMSQKFINEDKETTKKNE

SEQ ID NO: 553 - protein, 451 aa, unidentified
  source 1..451: note = Uncultivated organism; MG119-215 effector
MVTTKKIQLIPVGDPEEKKRVYEYLHDGIYNQCCILNTYMSQVATIYYKYNKDFKNADFKNEYNAIFRNTNTAIKDFQQAKGLGMAGNCGMRVKQDFSTALKNGLARGERSIPYYKRDFPLLCPSRFLTFYTGEDSFLNDKDEEVSVDAFFIKFVNGIHFKCVLGSKNKRRADLYLPELLSSIVEDPEHYHVCGSSIQFHGTKIILNLAVKIEKDAETYNPVEGRTMGLAMGYNKCLVAALSDSDRQYNIGDSNNPEYDLSRQIVTKRVEIQEHRTSLQKSLKGAAGGHGRSKKLKALDTQKKHENNVIRHFNHLMSKSVVDFAKKNKVEVIIMESIEKKDLDDVFLRNWSYYQLQSYITYKAEREGIRVIMSNATSASDKKKKGKKKDTTEETVMKTIHNTCCMCGKEIKREDILPEKIVWANAVSFTCPHCGNHLDYSFNKAKNMTTMG

SEQ ID NO: 554 - protein, 417 aa, unidentified
  source 1..417: note = Uncultivated organism; MG119-216 effector
MKNTFVTRYRVLPVGEKEERDRIYQYYRDAQYNQYLALNQAMSMAGIIYYNCHRNIKSEDFKTQYKDMFKRNSTVWDGVDFPKGGHLQGNVSRKVQSDFSTALKKGLAKGERQLPRYRRDFPYMIHGAEIKPYAQEEEYEGKTRTAYYIKSVNKIHLKFVFGSAERKQYRKVDMIKKLAEEDPAYKICQSNLFFKDNKMFLNLVVTKDVETKKYDPVKGRIMGVSYGFNCPAQTAFSDSENTYSLGDGDVFVEKRIQLQKNYNSLQKALRNAGGGKGRKKKLRALERYKAKEKNFAKQYNHKLSRQIVDMAKEKKVEKIILQQIDTKKLKKNPVLLRNWSYSQIYEMIKYKAEYGGIETDIRPAEDICPCCGCFIELPEFDNEMYEKEFVELCTECGEKINVSQNRAQYLAKQIGEN

SEQ ID NO: 555 - protein, 470 aa, unidentified
  source 1..470: note = Uncultivated organism; MG119-217 effector
MPTLTRKVELYVVGDKEEVSRVYDYIRLAMNATYKCFNECMTALYIAQVKEDTKEDRKELNYLYSRQTYTKKETAFTNDIVFPTGLVRASYINHEANLKFKTSMKNGLMYGCVSLPTYKKDCAVPLRCEFVSFAENKDKGLGFYHEYADVNELVNALEYDNNPKVFLKFPNNIRFEVVFGNPYRGREQRSVFSKIFLGEYKTCGSSIQINSKGRIILNLTMEVPKKEMEQIGGRVVGVDVGLAIPAMCAINDDNYARSAIGDINDFLRVRTQIQNQRRRLQKSLKNTSSGHGRAKKLKPLERISERERNFVNTYNHMVSKRVVDFAVKNGASQINIEDLSGFAKDKNGKSVEDANMKKILSNWSYFELQQQIRYKAGQYDIKVRTVNPAYTSQTCSYCGQIGKRETQSKFVCTNPDCSCHKMYKKDWFNADFNAARNIALSTDYTDDEDGKKTKKKKSAKKKLEKKTEEA

SEQ ID NO: 556 - protein, 453 aa, unidentified
  source 1..453: note = Uncultivated organism; MG119-218 effector
MLEAQTITRKIQIIPVGDKDEVNRVYTYLREGIKSQNMAMNQYMSALYSAMVADISKDDRKELNKLFSRISTSKLGSAYDETIEFAKGLPMGGAIQQKVKRDFDNAMKKGLKYGKLSLPTYRDNNPLLVHVDYVRLRSTNPHTDNGLFHNYESHEDFLDKLYSKDLELFIKFANKITFQIILGKPHKSAALRSEIKNIFEDYYHVKGSSIQIDGTKIILNMSLEIPKQKGVELDENIVVGVDLGIAIPAMCALNTNNYVRLRIGSRDDFLRVRTQIKNQRKRLYSNLKFANGGHGRNKKMQAANRFTDYEKNWVQTYNHMVSKRVVDFALKHKAKYINLECLDGFSQKDLKDNYLLANWSYYQLQQYIKYKAEKYGIVVRFVNPYHTSQNCSCCGHWEEGQRIKQAEFICKNPECKNFGKKINADFNAARNIAMSTYFIDKEEKKKKKTKKAA

SEQ ID NO: 557 - protein, 442 aa, unidentified
  source 1..442: note = Uncultivated organism; MG119-219 effector
MPNLTRKYKLIPVGDKEEVNRVYTCLRKDMENQNKAMNQYMTALYVAQIREASKEDRKELNNIYGRIATSKKGSAYTNDIVFTKGLDILSMAKRKVSEDFGNAMKKGLMYGRISLPTYRQDNPLLIRPDFVRLRSLSLKDYGIYHKYDSPMDLINGLEKDTNPEVYIKFPNGITFKIIFGNPHKGREQRKVFENIFSETYKVCGSSMGIDKNRKIILNLCLEVPKQEHKLDENVIVGVDLGQAIPAMCGLNNNYQIREKIGDKENLLHVRNQMRAKRERLQENLKFSKGGHGRKEKLKALDRLKDRERHFVQTYNHKVSKRVVEFALRHNASQINIEDLSGFGKDKNGNVEELKKKILSEWSYYELQNYITYKAQLHGIVVKKVNPAYTSQTCSCCGQMGIRLSQSEFICQNPDCKCHTLYGKYINADFNAARNIAMSTDYR

SEQ ID NO: 558 - protein, 446 aa, unidentified
  source 1..446: note = Uncultivated organism; MG119-220 effector
MGVSCKKLQIVPVGDDNEVKRVYEYIRNGIYSQYRILNTYMSQVGTLYYKYDRDYKDPGFKDEYNAIFRNTNTAIHDIEQAKGLGMAGNCGMRVKSDFSSALKNGLARGERQLPFYKRDFPLLVPGRFLVFSEKEEDYYDSNGELKTVVNYYIKFVNGIIFKVVFGSKGRSDYRLTSLLSGIVNDVDHYKVCGSSIQISGKKIILNLTTNVEKETVPFIPIKDKKMGIVLGYEQPLVAALSNDDKEYYIGTDIAESIVERRTAIQAYRHRLQVMQTTAKSGHGRKRRMRRVDQQGHYEKNVMRNYNHILSSQVVSFAVKNKVETIFIDDISKDTLIDEPILLRNWSYYQLVEFITYKAKEVGITVITVDFKKNKKEDGEDEADASNKHDFFDRCCVCGEQIDGLAELYDDISVYKDESSFTCPHCRKKVKVRYNKAKNLTYYKNKK

SEQ ID NO: 559 - protein, 452 aa, unidentified
  source 1..452: note = Uncultivated organism; MG119-221 effector
MTETQTITRKIQLIPVGDKEEINRVYTYLRDGIKSQNMAMNQYMSALYSAMVVDVSKDDRKELNHLFSRISTSKLGSAYDETIKFAKGLPSASSVAQKVKQDFDNAMKKGLKYGKLSLPTYRDNNPLLLHVDYVRLRATNPHLDNGLYHNYESHEDFLDNLYSKKLELFIKFANKITFQIVLGNPHKSAEIRSVFKNIFEDYYHIKGSSIEIDGTKIILNMSLEIPKQKVELDEGVVVGVDLGIAIPAMCALNTNDYVKLRIGSKDDFLRVRTQIKNQRRRLYSNMKFANGGHGRTKKMQAADRFADYEKNWVQTYNHMVSKKVVDFALKHKAKYINLECLDGIGQNDLRNNYLLANWSYYQLQQYIKYKAEKYGITVRFINPYHTSQNCSCCGHWEEGQRIKQAEFICKNPECKKFGKKVNADFNAARNIAMSTDFVEKEEKKKKKTKKAV

SEQ ID NO: 560 - protein, 436 aa, unidentified
  source 1..436: note = Uncultivated organism; MG119-222 effector
MITRKIQITPVGDKKEVDRVYAYLRNAIEIQNKAMNEYMSTLYIAEQSELTKDDRKELHNLYGRISTSKKGSAYDKSMEFPTGLPLGVITQAVEADFKKAKKDGVMYGRTSLPTYRADNPLNIHVDYCRLRKLSSHDCGLYHNYKTHLEFLDHLYKKDIEVFLRFANGITFKLNLGNNLQKSYALRVEIQKIFEEEFDVRGSKIQLKKRKTGKGDDILLLLSIETPKKEYKLDENIVVGVDLGVATPAVCALNNKWQPRKYIGDGQTFIHQKQRHKEQRQAIQRNLALCSGGHGRKKKLQKLDKLKQHEGNFTTNYSHKISKEIVDFALKHNAKYINIEDLTGIDTQHNKLLGKWKYFEVQEFTKYKAAQYGIEVRKIVPKYTSQTCSVCGHWEEGQRNGRNFECKNCGTKLNADYNAARNIAMSENFSEEKKKKK

SEQ ID NO: 561 - protein, 465 aa, unidentified
  source 1..465: note = Uncultivated organism; MG119-223 effector
MPNLTRKYELQVVGDKEEINRVYTWLRQGIEAQNKAMNEYMSHLFMAKSCEMSLDDRKELNNLFQRVTNSKKGSGYTKDIEFPVGLPIAASASRKVKQDFDNACKKGLMYGRISLPTYRSNNPLIIHVRFVALRGTKKVDNGIYHNYNSPADLFSALENDAKPEVFIKFCNNITFKFRFGNPYKAKEQREVFKKIFTEEYKVGGSTIEIKGTKIIINLTLEVPQIKMCLDENVTVGVDLGQAIPAMCALNNDYYKRLAIGNVDDFLRIRTQMQEERKRLQCHLRNANGGHGRKKKLRALERLKQREKNFVQTYSHMVSRKVVDFAVKHNAKYINIEDLSGFGRDKYGHVKDDEQAKMVLRNWSYYNLQEKIKYKANIYGIEVRKIDPKFTSQICSVCGEKGIRQTQKEFICINPECSCHKMYQNQKYINADFNAARNIAMSQKFINEDKETAKKNEQKKENSSSN

SEQ ID NO: 562 - protein, 428 aa, unidentified
  source 1..428: note = Uncultivated organism; MG119-224 effector
MSVTTKKIQLLPFTDSKEERERIYNYLRDGIYNQHHILNTYMSQMGVVYYKYNKDIKNPDYKEEVKAIFRNTNMSIKDFTQAKGLAMAANCGMQVKSDFSIAIRNGLAKGERALPFYKRDFPLLVPSRFLNFYSTTESYKDDFGEIKTRDIYAIKFVNGIHFKVVLGSRGRNDFYINSLLDSILNDPDNYHVCGSSIQFSKGGKIILNLTVKISKQAEEYEPVKGRVMGLAMGYDKCLVAAISDKDDAYSIGDYIQESIVEERIKIQEYTQRLQRSMKSAQGGRGRKRKMRSFDKKGSHEKNVVRHFNHILSKTVVEFAKKHKVETIVIEDVNEKELKDHPTLLRNWSFYQLITFISYKAKAYGISVEKSSSKDKPRMQCCFCGEALDKEDIIPKEIEWQNELYFTCPKCGKRIEYSFNKAKNMTVMG

SEQ ID NO: 563 - protein, 453 aa, unidentified
  source 1..453: note = Uncultivated organism; MG119-225 effector
MTSKASQNNTLVVFRKVRLIATGEKSDVNETYKYIRDKMYAQYRVRNLIAGHLYRAYYNAGCNDESPEFNAAADQILSNTSPILDVLDVEYDAKMVEFACQKVSEDFLSAVKNRLEIGTCRFINYKRTQPLQIDTEQVTFHHNYSNAEEFGANKLSEDAEVTVELAQKYKLKAQLGLPYRSGEIRSVFYNLFAGEYKLLSTSIAIDDKAIILTMSIETPYVSRGKKSYQGDADTSVGVILLKYGQAVCAVNNNNYARCYIGDKNHREYVGNFLHRKEKYQARIAECSEKIALYNQMEFIGKTNKIRALKREIEKYEKNIQNLDHAKQRYQSRTYDGIARSVVNFATKNHAKTIRVGLHSEASAPFYIREVIKRLKEKAADYKIELVVHECDCFEQKCSYCGEPLSLLMEDSRLLHCNNPDCFVNKVPARSKTSSGAIEKNFNAARIVAKLSNN

SEQ ID NO: 564 - protein, 428 aa, unidentified
  source 1..428: note = Uncultivated organism; MG119-226 effector
MSVTTKKIQLLPFTDSKEERERIYNYLRDGIYNQHHILNTYMSQMGVVYYKYNKDIKNPDYKEEVKAIFRNTNIAIKDFTQAKGLAMAANCGMQVKSDFSIAIRNGLAKGERSLPFYKRDFPLLVPSRFLNFYSTTESYKDDFGEIKTRDIYAIKFVNGIHFKVVLGSRGRNDFYINSLLDSILNDPDNYHVCGSSIQFSKGGKIILNLTVKISKQAEEYEPVKGRVMGLAMGYDKCLVAAISDKDDAYSIGDYIQESIVEERIKIQEYTQRLQRSMKSAQGGRGRKRKMRSFDKKGSHEKNVVRHFNHILSKNVVEFAKKHKVETIVIEDVNEKELKDHPTLLRNWSFYQLITFISYKAKAYGISVEKSSSKDKPRMQCCFCGEVLDKEDIIPKEIEWQNELYFTCPKCGKRIEYSFNKAKNMTVMG

SEQ ID NO: 565 - protein, 473 aa, unidentified
  source 1..473: note = Uncultivated organism; MG119-227 effector
MSVSCKKLQLIPLGDKEERNRVYTYLKNGIYSQYRILNTYMSQVGTLYYSCNRNVESEEFTSGFDQIFKDTEAAYRNLGFMAFGDYLRKMEEDYKKHMKDLKDKEYKESYNAIFRNTNEAIWDIEQAKGLGMAGNAGMRVKQDFSTALKNGLARGERQLPFYKRDFPLLVPSRFLTFYSEDEVYTNEQTGEECERTNYCIKFVNGIRFKVVIGSQGKRDLYLLPLLDSILHDPENYKVCGSSIQITKQGKIILNLTTNINKREKEYEPVKGRVMGLSMGYDKCLVAALSDKDKMYEIGTYDKDGYIERRIRLQNERQLLQMKKKGPEGSGHGRKRKMRDFDNMRKRESNIVRYYNHRLSKQVVEFAKKNKVEAIVIEEINASDLNEHPTMLRNWSYYQLQDFIKYKAGKGIKVMVSSKEENRKVCCNCGKPFEKKDVLPKEIEWTGTIHFTCPNCKKQIEYSFNKAKNMALNG

SEQ ID NO: 566 - protein, 446 aa, unidentified
  source 1..446: note = Uncultivated organism; MG119-228 effector
MGVSCKKLQIVPVGDDNEVKRVYEYIRNGIYSQYRILNTYMSQVGTLYYKYDRDYKDPGFKDEYNAIFRNTNTAIHDIEQAKGLGMAGNCGMRVKSDFSTALKNGLARGERQLPFYKRDFPLLVPSRFLVFSEKEEDYYDDNGELKTVVNYYIKFVNGIVFKVVFGSKGRRDYRLTSLISGIVNDVDHYKVCGSSIQISGKKIILNLTTKVEKETVPFIPIKDKKMGIVLGYDQPLVAALSNDDKEYYIGTDIAESIVERRTAIQAYRHRLQVMQTTAKSGHGRKRRMRRVDQQGHYERNVMRNYNHILSSRVVSFAVKNKVETIFIDDISKDTLIDAPILLRNWSYYQLVEFITYKAKEVGITVITVDFKKNKKDDGEDEADASNKHDFFDRCCVCGEQIDGLAELYDDISVYKDESSFTCPHCRKKVKVRYNKAKNLTYYKNKK

SEQ ID NO: 567 - protein, 452 aa, unidentified
  source 1..452: note = Uncultivated organism; MG119-229 effector
MTETQIITRKIQLIPVGDKEEINRVYTYLQDGIKSQNMAMNQYMSALYSSMVVEISKDDRKELNKLFGRISTSNLGSAYDKTIEFAKGLPMGGAIQQKVKRDFDNAMKKGLKYGKLSLPTYRENNPLLVHVDYVRLRTTNPHSDNGLYHNYESHEDFLNSLYSKDLELFIKFANKITFQIVLGNPHKSAEIRSVFKNIFEDYYHIKGSSIQIDGKKIILNMSIEIPKQKVELDENVVVGVDLGLAIPAMCALNTNDYVRLRIGSRDDFLRVRTQIKSQRRRLYSNLKFAHGGHGRSKKMQATDRFTNYEKNWVQTYNHMVSKRVVDFALKHKAKYINLECLDGFSKKQLRDNYLLANWSYYQLQQYIKYKAEKYGIVVRFVNPYHTSQNCSCCGHWEEGQRVKQAEFICKNPECKNYGKKINADFNAARNIAMSTDFVEKEEKKKKKTKKAA

SEQ ID NO: 568 - protein, 465 aa, unidentified
  source 1..465: note = Uncultivated organism; MG119-230 effector
MPNLVRKYELQVVGDKEEINRVYTWLRKGIEAQNKAMNEYMSHLFMAKSCEMSLEDRKELNNLFQRVTNSKKGSGYTKDIEFPIGLPIAASASRKVKQDFDNACKKGLMYGRMSLPSYRNTNPLIVHVNFVALRGTKKPDNGIYHNYENPAKLFEALENDMKPEVFIKFCNNITFKFRFGNPYRGKEQREVFKRIFTEEYKVGGSTIEIKGTKIIINLTLEVPKVEAVVNEDVVLGVDLGQAVPAMCALNNDYYKRLAIGSADEFLRIRTQIQNERRRLQHALKDSKGGHGRQKKLRALDRLKEREKNFVQTYSHMVSRRVVDFAIKNGAKYINIEDLSGFGRDKYGHVKDDEHSKMILRNWSYYDLQEKIKYKAAIHGIEVRKIDPKYTSQTCSVCGEKGLRQTQKEFICTNPECVCHKIYSNQKYINADFNAARNIAMSQNFIEEDEGSKKKSEQKKENSNGN

SEQ ID NO: 569 - protein, 429 aa, unidentified
  source 1..429: note = Uncultivated organism; MG119-231 effector
MVITRKIALTVVGNKEEKDRVYTYIRDGIKNQNLAMNQYMSALYVANMQDISKDDRKELNHLYTRISTSKKGSAYSTDIQFPKGLPCTSSLGQEVRAKFKKACKDGLMYGRVSLPTYRANNPLLIHVDYVRLRSTNPHNDTGLYHNYESHTEFLEHLYKNDCEVFIKFANNITFQLFFGQPHKSHELRSVIQKVFEEYYSVCGSSIEISKKGKIMLNMCIEIPVEKKELDENIVVGVDLGISTPAMCGLNCNDYVREGIGSKDTLLSKRTQLQRQYRELQGRMKMTNGGHGRGKKLKKMDDYRNHERHFVQTYNHQVSKKIVDFALKYKAKYINVEDLSGFGNRDTNQWVLRNWSYYELQQYITYKAQKYGIEVRKVKPYLTSQTCSHCGHYEPGQRLDQAHFECKNCGLKINADFNASRNIAMSTEFV

SEQ ID NO: 570 - protein, 451 aa, unidentified
  source 1..451: note = Uncultivated organism; MG119-232 effector
MSNTQILTRKFQIFPVGDKEEINRVYQYIRDCQYVQNKAYNILISNVYSAIISGKSKEEINLIYKKGGRKPKEDNPDYSLYNYNDFKVPVGLTIIGALPQTVKNDLKTAQKAGLFKGNSALPNRRRTASLNIQPVYVRLRDINLRNPKITFKTGIYHTYSSYNDFLDNLYSKRELDIYLAFANNITFKFVLGNPYKSYQDRNILQHIFEEDYLVKGSSIFFDKTNKKIMLNLSMEIPKKEIELKEDVVVGVDVGLAIPAVCGLNINSYAREYIGSKDDFLRVRTQISNERKRVQKNLKMVKGGHGRKKKMKSLDRFTLKEKNFVQTYNHMVSKRVVDFAVKNHAKYINLENLKGYDANIFILRNWSYYQLQQYIKYKAEKYGIETRMIDPYHTSQICSCCGHWEEGQRINQSTFICKNPECKNFGVKINADFNASRNIALSTNFISADSAE

SEQ ID NO: 571 - protein, 477 aa, unidentified
  source 1..477: note = Uncultivated organism; MG119-233 effector
MAVLTRTLEMMPVGDKAERDRIYTWIRSFMDAQSRMMNQYMSALYIAAIQEISKEDRQELNKLYNRIPTSSKGSAFDKEIELPKGLGAAYGQTVRADFDTSCENGLLHGRVALPTYKKDYPIIIPPLYVQLRKVNQETKGKNCGFYEEYGSYNDLYDSLTQGEPEIFWSFVDKMIFKIRFGNPYKSAFLRSEILHFLEGEYKAMGSSLSINNKGKIILSLTMEIPEKKVKLDENTVCGVSLGFMHPIVCSVNNNFYKKEFIGDYDVFVDTRVRLQDQRRELQKKLKYASSSHGRVKKLRKLAILRKREANFAKNMNHKMSSDIVKFAVKNEAKYINMQDLTSFGKDSRDNIKSGYEFALRNWSYFQLQQMVTYKAASYGIIVRKISNDNLSNTCSICGAEGIIEDGAFICSDPNCLSHDKYEKARKNGKGNYYFNSDFNISRNVSISENFTTKKRKSKAEKKEERKAKKEKEKLDKS

SEQ ID NO: 572 - protein, 468 aa, unidentified
  source 1..468: note = Uncultivated organism; MG119-234 effector
MEEKKTHVISRKVRLYVVPHEGTPEEQEAEINRVYTELRSGMEAQNKAYNILVSAIYSATMSGASMDEIKMICKRGSRNPKEDDPDYSLYDFNEIKFMKGLPSASKVNRYAEADMKKAWKDGLKYGVVSLQTKKMDAPLWVIPENLKYRHNCPSYTLFLELLMNGKTDIFIDFVQGITFGVVLGNPYKGHRNRLMWKRIFEGEYKIGTSSIQIAKEGKIILNLALTVPVEEHKELNKDIVVGVDLGIKIPAVCALNNNEYEREMIGSINDFLRVRTSMQAERKRIKKALKTGASGGHGRKKKLKNLDNIERREHNFATTYNHMVSKRVVDFALKNNAGVIHIENLEGFGKNEKTQKVLRNWSYYQLQQDIIYKAEANGIEVKKINPYHTSQICSICGSEEPGQRTSQTTFICANPKCRSHKIYFNDRHGKEEFNADFNAARNIAKSTEYQTGKKSKRQKKEQHENING

SEQ ID NO: 573 - protein, 447 aa, unidentified
  source 1..447: note = Uncultivated organism; MG119-235 effector
MSTTTKKIQLIPVGDKEEKDRVYDYLRDGIYNQHKILNTYMSQVGTIYYKYNKDFNNPDFKEELKAIFRNKNEAIYEFTQAKGLGMAGSCGRRVNQDFSTALRNGLARGERALPFYKRDFPLLIQGRFLTFYTGEDSYQDEEGNEKTVNAYFIKFVNGIHFKCVLGSRGRKRPDLYLPRLLQSIVEDPENYRICGSTIQFSKDNKIILNLTVDINKTVEDYKPVKGRVMGLTMGYDKCLVAALSDSDEMYTIGDYIKDSVIEKRIKIQKDTHRLQMSMREDAKGGHGRKRKLEKLEQHKKYEKNVIQDFNHRLSKSVIDFAKKHNVEIIVLEGISKEELEKYPTLLRNWSYYQLQTYIKYKAKAEGIDVRVIEDKQADTDDKTQKGRKKKKALDEIVRKTCCACGCSLDRDDIIPKQIEWCHELSFTCPECGKTIDYSYNKAKNMIS

SEQ ID NO: 574 - protein, 403 aa, unidentified
  source 1..403: note = Uncultivated organism; MG119-236 effector
MNEIKMICKRGSRNPKEDDPDYSLYDFNEIKFMKGLPSASKVNRYAEADMKKAWKDGLKYGVVSLQTKKKDAPLWVIPENLKYRHNCPSYTLFLELLMNGKTDIFIDFVQGITFGVVLGNPYKGHRNRLMWKRIFEGEYKIGTSSIQIAKEGKIILNLALTVPVEEHKELNKDIVVGVDLGIKIPAVCALNNNEYEREMIGSINDFLRVRTSMQAERKRIQKALKTGASGGHGRKKKLKNLDNIERREHNFATTYNHMVSKRVVDFALKNNAGVIHIENLEGFGKNEKTQKVLRNWSYYQLQQDIIYKAEANGIEVKKINPYHTSQICSICGSEEPGQRTSQATFICANPKCRSHKIYFNDRHGKEEFNADFNAARNIAKSTEYQTGKKSKRQKKEQHENING

SEQ ID NO: 575 - protein, 448 aa, unidentified
  source 1..448: note = Uncultivated organism; MG119-237 effector
MITRKIQLYPIGDEKEVDRVYTYLRDSIKSQNKAMNQYMSNLYVEELLNASKEDRKELNALYTRIPTSKKGSAYSLDLEFQKGQMLGYITRAIDSDFKKSLKDGLMYGKVSLPVYSNSNPLLVHVNYCNLRSKVQKQTGTNTGIYHNYESHQEFLSHLYKTDIEIYLQFCNNILFKLNLGDSLRKSREIRSVIQKIFEEEYIIHGSQIQLKKRKTGKGEDIFLLLSIETPVVKHELDENIVVGVDLGVANPAVCAVNINGNYKFLGDGETFLHYKQKSKAERRRLQRALKYTNGGHGRTKKLKALDRLKERESNFTTTYSHKLSKDVVDFAIEQHAKYINLEDLTGIDDKDGKKLLDCWKYYELQQFIIYKATHHGIVVRFINPKYTSQTCSKCGNHAEGQRDGHSFTCKDCGEELDADLNAARNIAMSTNFVERGKKKKKKNKTDNK

SEQ ID NO: 576 - protein, 439 aa, unidentified
  source 1..439: note = Uncultivated organism; MG119-238 effector
MGTTTKKLQLYPIGEKEEVDRVFTYLRDGIYNQHCILNTYMSQVGCLYYKYNKDFKNVGFKEEFNAIFRNTNEAIKDFTQAKGLGMAGNCGMRVKQDFSTALKNGLARGERNLPFYKRDFPLLVPSRFLTFYTIDSVDKKTGEERTVYCIKFVNGINFKIVIGSQGKKDFYLLPLLDSIINDPDNYHVCGSSIQITKKGKIILNLCVKVSKKAEEYHPKQGRVMGLALGYDKCLVAALSDSDKMFTIGDDIKDSVVEKRIKIQAYNQALQKALKDAKGGHGRARKLAKFNERDRYEKNVVRHFNHVLSKKVVEFAKKQKAETIVIENIDKADLESYPVLLRNWSYYQLCEYIKYKSGDDINVILAGEGKKSKKKAEGDTDTATSAELRKICCKCGCKLDESVILPEKIEWTNELYFTCPECKEKIEFSYNKAKVMSMKG

SEQ ID NO: 577 - protein, 467 aa, unidentified
  source 1..467: note = Uncultivated organism; MG119-239 effector
METILTRKIMLKVVGDNAEKNRVYSYLRNGQYINALMKNQYISALYAAYMTGASKEEIKELNTLYSRVPGSGKGSAYSFDTKLYPTGLPLASSVQHLGKDLFSKACKDGLLHGKVSLPTYKADGPMPIPKYLVSRRGTVHRTSGNKEGFMDAGFYHEYASDAEFFSELQSKRVPDVYIKFTNNIVFKVRFGNKNRTDQERALISDIFAGRFPVCDSSIGVEDKDIILNLSMKIPECKNELDKDTIVGVKLGIATPAVCALNNDYKKREYIGTYEEFTGHRNKMAARKRILTRDLAQCKGGHGRSKKLAKLEKNSAREADYAKTYNHRISRTIIDFALKNKASVIQIEKLSSLNKKKKKYFDDTKEKKEERNLLGNWSYYQLQQQIEYKAKKEGIKIRYVNPAYTMQTCSCCGMLGERVGNDFRCISSGCKFNGKIRNADFNAARNISMSTDYIDNEKETAEESEETA

SEQ ID NO: 578 - protein, 442 aa, unidentified
  source 1..442: note = Uncultivated organism; MG119-240 effector
MKYINVVRKIQAVPCGDRNEANRVYQYLRSGTEAQNKAMNQMMNGLVMAHVSEMQKDDKKEFVKYFNRTGTTKLESAYVDVEFATGIPISAFVQKVKADFDTALKNGAAYGRVRFPEYKSDNPLLVHIDWVRLRKTNPHNDFGIYHCYPTDEEFEKHLYSKDLEIFWKFANGITFKLVFGNLKRSCELRSVFKNIFNETYEIRGSSIQINSKNQIILNMTIKMPVYEASLDENTVVGVDIGMAIPAMCALNNNPVCRQRIGSFDDFMRVKTQIKKERSRIQSSIRFGSGGHGREKKLKSMQKFRSRESNWAMTYNHTVSRKVVDFALKNHAKYINLEDLSGIASQEKSNKLLRDWTYFQLQQFITYKAEKVGIVVRKIDPYHTSQICSCCGHWEEGQRISQDKFICKNPECKNFGKIVNADFNAARNIAMSTAFVDEKKLSA

SEQ ID NO: 579 - protein, 465 aa, unidentified
  source 1..465: note = Uncultivated organism; MG119-241 effector
MPNLTRKYELQVVGDKEEINRVYTWLRQGIEAQNKAMNEYISHLFMVKSCEMSLDDRKELNNLFQRVTNSKKGSGYTKDIEFPVGLPIAASASRKVKQDFDNACKKGLMYGCISLPTYRSNNPLIVHVRFVALRGTKKVDNGIYHNYNSPADLFSALENDTKPEVFIKFCNNITFKFRFGNPYKAKEQREVFKKIFTEEYKVGGSTIEIKGTKIIINLTLEVPQIKKCLDDNITVGVDLGQAIPAMCALNNDYYKRLAIGNVDDFLRIRTQMQEERKRLQCYLRNANGGHGRKKKLRALERLKQREKNFVQTYSHMVSRKVVDFAVKHNAKYINIEDLSGFGRDKYGHVKDDEQAKMVLRNWSYYDLQEKIKYKANIYGIEVRKIDPKFTSQICSVCGEKGIRQTQKEFICINPECSCHKMYQNQKYINADFNAARNIAMSQKFINGDKETTKKNEQKKENSSSN

SEQ ID NO: 580 - protein, 450 aa, unidentified
  source 1..450: note = Uncultivated organism; MG119-242 effector
MGQNITRKIQLVPIGDKKEISRVYNYLSKHIEAQNKAMNQYISSLYAAHLLDISEEDRKELRNLYSRIPTSKKGSGYDTSIEFQKGIPLSNITRSIESDFKKSLKDGLMYGKVSLPTYKKNNPMLVHVNYCDLRDKVKERTGKNTGIFHNYKNHEEFISHLYKTDLEVFLQFTNNILFKFHFGESVKKSSEIRSVMRKIFEREYTIHGSQIQLKERKTGDGDDIFLLLCIEVPEVTHELSEDTVVGVDLGVANPAVCALNTKRYPYKYIGDGSIFIHHKQKYKNERTRLQRNLKYTSGGHGRKKKLRALNRLRDSESNFATTFSHKISKDIVDFAISNNAKYINIEDLKGIDTKNTKLLECWKYFELQQFITYKAARHGITVRKINPEYTSQTCSICGNREDGQRDGRNFHCKACGAELDADYNAARVIAMSTNFVEDKKKKKKKKEESE

SEQ ID NO: 581 - protein, 469 aa, unidentified
  source 1..469: note = Uncultivated organism; MG119-243 effector
MPTLTRKVKLHVVGDKEEVSRVYDYIRLAMNATYKCFNECMTALYIAQVKEDTTDDRNELNCLYSRQTYTKKETAFTNDIVFPTGLALASYINHEANLKFKTSIKNGLMHGCVSLPTYKKDCAVPLRCEFVSFAENKDKGLGFYHEYADVNELINALEYDNNPKVFLKFPNDIRFEVVFGNPYRGREQRSVFSKIFLGEYKTRGSSIQINSKSRIILNLTMEVPKKEMEQIEGRVVGVDVGLAIPAMCAINDDNYARSAIGDINDFLRVRTQIKSQRRRLQKSLKNTSSGHGRIKKLKPLEKIAEKERNFVNTYNHMVSKRVVDFAVKNGASQINIEDLSGFAKDKNGKSVEDANMKRILSNWSYFELQQQIRYKAGQYDIKVRTVNPAYTSQTCSYCGQIGKRETQSKFVCTNPDCGCHKIYKDGWFNADFNAARNIALSTDYTDDEDGKKSKKKPAKAKVKKWNEKD

SEQ ID NO: 582 - protein, 444 aa, unidentified
  source 1..444: note = Uncultivated organism; MG119-244 effector
MNNIVTRKIGLFVVGDKTESDRVYKYLRDGMASQNRAMNQYITALYFADQNEATKDDRKELNRLFSRISSSKKGSAYDTSIEFAKGLPSGSSVTQKVEADFKNAMKKGLKHGIVSLPTYKKDNPLIIHRDYVRVRFTNPHHDNGLFHEYESHEDFLKHLYSNDLDLFIKFANGITFKLYLGKPKKSAELRSVLKNIFEDYYQIQGSTIEITNGKIILNLSVSIPQKEIELDENTVVGVDVGMACPAVCAVNKNDVYKQFIGSADDFIRVRTQIQNEYRRLKQSVAFNSSGHGRSKKAKPTDRFHEYESNWVKTYNHKVSKKVVDFALKNKAKYINIENLSGIAQDEKNKAFVLRNWSYYQLQQFITYKAAKYGIIVRKVDPAYTSQRCSCCGHLEKGQRVSQSDFICKNPKCKNFGKIVNADFNAARNISMSTKFVKDDVEVGT

SEQ ID NO: 583 - protein, 451 aa, unidentified
  source 1..451: note = Uncultivated organism; MG119-245 effector
MSNTQILTRKFQIFPVGDKEEINRVYQYIRDCQYVQNKAYNILISNVYSAIISGKSKEEINLIYKKGGRKPKEDNPDYSLYNYNDFKVPVGLTIIGALPQTVKNDLKTAQKAGLFKGNSALPNKRRTASLNIQPVYVRLRDINLRNPKITFKTGIYHTYSSYNDFLDNLYSKRELDIYLAFANNITFKFVLGNPYKSYQDRNILQHIFEEDYLIKGSSISFDKTNKKIMLNLSMEIPKKEIELKEDVVVGVDVGLAIPAVCGLNINSYAREYIGSKDDFLRVRTQISNERKRVQKNLKMIKGGHGRKKKMKSLDRFTLKEKNFVQTYNHMVSKRVVDFAVKNHAKYINLENLKGYDANIFILRNWSYYQLQQYIKYKAEKYGIETRMIDPYHTSQICSCCGHWEEGQRINQSTFICKNPECKNFGVKINADFNASRNIALSTNFISADSAE

SEQ ID NO: 584 - protein, 458 aa, unidentified
  source 1..458: note = Uncultivated organism; MG119-246 effector
MSKEKTLIMVRKVKLHPVGDKEEVSRVYKYIRDCSYVTKIVKNKSMSFLYSAFVTGMDNEKRKEEYRKYTRKPNPNKEGYSAYEGIELPDIKGLPVFGDASHAAQDEFKNDLKNGLASGKISLRNTKQGVPVNINKCYLQKRDDPDYPGQKHGGFYHEYPDDREFVEHLDKDKVELYFQFPNNIYFRVDLGNPYNGKEIRSCFAKFWSGEYKTVGSKIMISKRNEIILSLAFEVPVENNDLNSDIVLGVDVGMAYPAYAALNNDNYARRSFGSFDELAKRKESFNAQRKRMQHGLSFAKGGHGRKRKLVKLDDLKTKEKNYTDTYIQQISSEIIRYALSKRAGVIQMEDLSFVREKSDDSVFVMRNWCYADLQSKIEYKAAKSGIEVRYINPAYTSKTCSVCGKAGQRLSQKEFICSDPDCKSHKMYRDNGKYFGADFNAARNIALSTDFSDKKKSRV

SEQ ID NO: 585 - protein, 470 aa, unidentified
  source 1..470: note = Uncultivated organism; MG119-247 effector
MATLTRKIQLYVVGDKKEVSRVYDYIRGATDATYKAFNECMTALYVAEKSDETKEGKKWLNEYYSRQTYTKYKSGFSDNIVFPEGLALASYINNLAKDKFSIAVRNGLMYGCVSLPTFKKDCAVPLHVKYVAVKGTKSDSHGLYHEYADVNDLINELENGDKPKVFLKFPSKITFGIVFGNPYDGREQRSVFSKIFLGEYQVCGSSIQINSKNKIILNLTLKVPDKKIEQVEGRVVGVDLGIAIPAMCAMNDDDYSRSSIGKINDFLRVRTQIRNQRMRLQKGLKNTSSGHGRTKKLEPLERFKEKERNFVQTYNHMVSKRVVDYAIKNGAAQINIEDLSGFGKDKNGKSKEDEKTKKILSNWSYFESQQYIRYKAEMHGIKVRTVNPAYTSQTCSYCGQRGKREEQSKFVCVNPDCKCHKMYKKGYLDADFNAARNIALSTDYTDDENGKKKKKKSSKKKDVNKSGKEE

SEQ ID NO: 586 - protein, 451 aa, unidentified
  source 1..451: note = Uncultivated organism; MG119-248 effector
MADTQILTRKFQIFPVGDNDEINRAYQYIRDCQYVQNKAYNILISNVYSAIISGKSREEIDLIYKKGGRKPKEDNPDYSLYNYNDFKVPVGLPIIGGLPQTVKSDLKIAQKAGLFKGNSALPNKRRTASLNVQPIYIRLRDINLRNPKINSKTGIYHTYSSYNNFLDNLYSKRELDVYLAFANNITFKFVLGNPHKSYQDRNILQHIFEEDYLVKGSSICFDKTNKKIMLNLSMEIPKTKIELKEDVVVGVDIGLAIPAVCGLNTNSYARESIGSKDDFLRVRTQISNEYKRVQKGIKMNKGGHGRKKKMKALDRFTLKEKNFVQTYNHMVSKRVVDFAIKNHAKYINLENLEGYDASKFILRNWSFYQLQQYIKYKAEKYGIETRMINPYHTSQICSCCGHWEEGQRIDQATFICKNPKCKNFGIKINADFNASRNIALSTNFISADSVE

SEQ ID NO: 587 - protein, 525 aa, unidentified
  source 1..525: note = Uncultivated organism; MG119-249 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKSMDEAAEYYGISEKYKA

SEQ ID NO: 588 - protein, 579 aa, unidentified
  source 1..579: note = Uncultivated organism; MG119-250 effector
MKKNNKESNTYIKAMKIRIVPICDIEEENLRDKYYKELYQYLRDSIWAQNAALNYGLAMTRDAYVLHKNEDKIKDIYFKLAHQTPNPSMASEARLKEVYEAAPITQEYIDNKVKEFKKSNNKKKKPLSDEGVKKKTDSINNMYKKFIGLSKEDIQGLIDKLDDYCAYPEEIYEKFANGFSTPAYITQQIKDYWNTEGVMTKVIYSMSDKLRSVKNSNPLTIPPNLFYNKKNDLIGITHSYNTYLEFVEALMNVYDANLFLELPYKKGYDKMKFKLILGNPCKSHEVRISLQRIFEEYYKIRGSKIGFVRNKKTGKNTDLVLYLSVEIPKDTSIELDENTVVGVDLGLAVPAVCALNNNPYPRKYIGKKLELLKKRTQFKNRRKKLQSELRDARGGHGRRRKMKAMDRLSELEKNFADTYCHKVAKKVVMFALKNKAKYINLEMLKGYRADEKVLQNWSYYRIQMYIKILAERYGIIVRFINPCYTSQVCSVCGNWHPGNRPKGDKGQKYFDCHNDKCESHTKNKGEGYTLNADYNAARNIAMCDLFMDNKSEITEEDKRIAREKYGIPEPIKKKEKLAA

SEQ ID NO: 589 - protein, 525 aa, unidentified
  source 1..525: note = Uncultivated organism; MG119-251 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTNYKKGFTADFNHARNVAMSTLFMENGSFSKKSMDEAAEYYGISEKYKA

SEQ ID NO: 590 - protein, 426 aa, unidentified
  source 1..426: note = Uncultivated organism; MG119-252 effector
MILTRKIQLYPVGDKEEVNRVYQYIRDGIFNQNKAMNQYMTALYVSTIQDISKEDRQELNRLYTRIATSKKGSAYDLDVEFAKGLPTTASLGQKVRQDFSKACKDGLLYGKVSLPTYRKDNPLLVHVDYVRLRETNPHMDNGIYHNYESYDEFISKLYDKDLEIFIRFANGITFKMILGQPHKSIYLREEIKKIFDGEYNVCGSSIQFNDKKIILNMSMDIPKISRELDENTVVGVDLGIAVPAVCGLNNSNVKKQFIGSKEDFLRIRTQLQAQRKRLQKNVKITNGGHGRNKKLKALDKLSNKERNFVKTYNHYVSKQVIDFALKNNAKYINMEDLSGFNTNKFILRNWSFYELQQFITYKAQRYGIEVRKVNPYHTSQICSKCGHWEEGQRDTQDHFKCKACGYEDNADFNASKNIAKSIDFRK

SEQ ID NO: 591 - protein, 414 aa, unidentified
  source 1..414: note = Uncultivated organism; MG119-253 effector
MANVLTRKIQLVPVGDKEEINRVYNYIREGQYSQYLGLNRLMSELVVTFYKNDRKLDEKFNEERKNILSNSNTILNDILFPKGCDTKSHIVQKVKSDFSKALKNGLARGEVAVTNYKRTSPLLVRGRDLKFSYDYATLNEFEQNLLNTDLKVYINFVDKIKFKIVFGNPHKSQALRLEIREILLDNYKVKGSSLQIDGKKIILNLSIAVPTKKIELDKKVTVGVDLGLAIPAVCALNTNDYIRKSIGSADDFLRQRTKIQKQKKAIQKNLVQVKGGHGRKKKLNHLEKVRKNEEHWVRTYNHFVSKQVVDFAIKNKAKYINVEDLTGFDNSKFILRNWSYYQLQEFITYKANNVGIQVRKINPYHTSQTCSCCGHWEEGQRISQSKFICKNYGKEINADFNAARNIAQCSDFIK

SEQ ID NO: 592 - protein, 462 aa, unidentified
  source 1..462: note = Uncultivated organism; MG119-254 effector
MGNDRMTICRKIKLFPVGDKEEINRVYDFIRNGQYAQYQACNLLMGQLMSEYYKYNRDIKNEEFKARQKEIMTNSNIILKDIDFATGVDTPSAVTQKVKQDFSTALKNGLARGERTVTNYKRTNPLITRGRNLTFYHEYETYHDFLDKINDSDLAVYVKWVNKIVFKVVFGNPHRSLELRSVVQNILEENYKVQGSSIEIDGKSIILNLSISIPKQLRELDENTVVGVDLGIAVPAMCALNNNLYERLAIGNAEDFLRIRTKMQAQRRRLQKSLRNISGGHGRAKKLKALERLQKAETHFVETYCHIISKRVVDFALKHNAKYINIENLTGYDTSDFILRNWSYYKLQDYITYKAAKYGIEVRKINPCYTSQICSVCGNWEFGQRKSQSVFECANENCDSHKKYEKIGFNADFNAARNIAMSTLWMESGQVTEKSKQEAREYYDISKKYEQSKSDSENNKVA

SEQ ID NO: 593 - protein, 441 aa, unidentified
  source 1..441: note = Uncultivated organism; MG119-255 effector
MVLTRKIQLIPVGNKEEINRVYKYLQDGIYSQNKAMNQYISALYVSAIQDISKEDRSELNKLYGRISTSKKGSAYDKNIIFPKGLPIGALTRKVRQDFSKSCKDGLLYGKVSLPMYKKDNPLLIHVDYVRLRKTNPHQDSGIYYEYDSYSDFLEHLHKNDLEVFIKFANQITFKMIFGSPHKSYTLREEIKQIFEENYKVCSSTIQIDGKKIILNLSMEVPKNDVELDENIVVGVDLGIAIPAMCALNNNEYIRKSIGSKDDFLRVRTQLQSQRRRLQKTLKISNGGHGRNKKLQALNKLKNRERNFVRTYNHYVSKQVIDFAVKNKAKYINVEDLSGFDSSKFILRNWSFYELQQFITYKAAKYGIEVRKVNPYHTSQICCKCGHWEEGQRIDQAHFVCKKCGNETNADFNAARNIAMSVDFIDKKKEVETTCNNMQLTA

SEQ ID NO: 594 - protein, 418 aa, unidentified
  source 1..418: note = Uncultivated organism; MG119-256 effector
MGDTQTITRKIQLFPVGDDEEISRVYKYIRDGMYAQYQASNLLMGQLASEYFKCNRDIKSDSFKNAQKEIFKADNPLLKEIEFPKGNDTLSAVTQKVKQDFSTALKNGLARGERTITNYKRTNPLLTRNRSLKFCYEYESYHEFLDKLFTNELEVFVKWVNKIKFKVVFGNPHKSHELRCVIQNIFEEVYKVQGSSIQFDNTGKKIILNLSLAIPRKRMELDENTVVGVDLGVAVPAVCALNNDEFAREFIGSKDDFLRVRTRIQAQKRRLQKSLKFSSGGHGRNKKLKPLKQFEDYEKNFVQTYSHMVSKHVVDFAVKHNAKYINIEDLEGYRSNDFILRNWSYYKLQEYITYKAGIYGIEVRKVNPYHTSQICSCCGHWEEDQRIDQLHFKCKLCGAELNADFNAAKNIAKSTDFR

SEQ ID NO: 595 - protein, 401 aa, unidentified
  source 1..401: note = Uncultivated organism; MG119-257 effector
MYLVRKIRLIVIGENKEDVNEKYKFIRDAQYAQYRGLNYMMGQVGALYYSCDRNISSDEFKTQYMEIFRASNQVVDGIEWGTGIDTRSLVGMRIKQDFSTALKRGLAKGEISLPNYKRSYPLLTKGRDIRFYTDYTEGELVHDMVLEPDFNLYIKWVHKIHFKVVLGNPYRSRFLREELWKVLCGEYKIAGSSLGFDKNGKLILNLSMDIPVNKEKKENMNNVLGVRLAYAKPVVAVVNSTGRCFMIGDGERLIGYRKKMRNNIRRVQQDVTLAAGGSGRAKKLAALNRFKDREANFAKTYNHQISRRIVEIAKNNGCSTIVLEDIGNIYDVDNIVLSNWGYYQLHQQIEYKAQMQDINVVKSQLVAEKQMQTMLETIELDKRDEFIAKSLSQIDIKQKKK

SEQ ID NO: 596 - protein, 426 aa, unidentified
  source 1..426: note = Uncultivated organism; MG119-258 effector
MILTRKIQLIPCTEGMTTDEAKVEVNRIYQLLRDGIYAQNKAYNIFISRMYTATLLGASKEELKEIRLKGERTPKESDSNYSLYDFDKIKFIKGLPQASALGEQALKALKKQQKDGLYKGKVSLACRKLDAPMWIKTKFSFFHKYNDYQEFLDHLYCDDLKIYMKFVQGIVFETVLGNPHKSETIRAEFQQIFEGHYKLCSSSLQIKDKKIMLNLAIDIPEKEIELNDETVVGVDIGIAIPAVCALNSREYIHKSIGSAEELLRIRTQLQSQKRRLQKNLKNTTGGHGRSHKLAPLDKLAKRERNFVQTYNHMISKTVVEFAVKNKAKYINLEDLSNYKDNGSEFILRNWSYFELQSQIEYKAKMHGIIVRKINPYHTSQICSKCGHWEEGQRISQSKFKCKSCGYESNADFNAARNIALSTDFIE

SEQ ID NO: 597 - protein, 457 aa, unidentified
  source 1..457: note = Uncultivated organism; MG119-259 effector
MTICRKIKLFPVGDKDEINRVYDFIRNGQYAQYQACNLLMGQLMSEYYKYNRDIKNEEFKARQKEIMTNSNIILKDIDFATGVDTPSAVTQKVKQDFSTALKNGLAKGERTVTNYKRTNPLITRGRNLTFYHEYETYQNFLDKINDSDLAVYIKWVNKILFKVVFGNPHRSLELRSVVQNILEENYKVQGSSIEIDGKSIILNLSISIPKQLRELDENTVVGVDLGIAVPAMCALNNNLYERLAIGNAEDFLRIRTKMQAQRRRLQKSLRNTSGGHGRAKKLKALERLQKAEAHFVETYCHMISKRVVDFALKHNAKYINIENLTGYDTSDFILRNWSYYKLQDYITYKAAKYGIEVRKINPCYTSQICSVCGNWEFGQRKSQSVFECANENCDSHKKYEKIGFNADFNAARNIAMSTLWMESGQVTEKSKQEAREYYGISKKYEQNKNDSENNKVA

SEQ ID NO: 598 - protein, 457 aa, unidentified
  source 1..457: note = Uncultivated organism; MG119-260 effector
MTICRKIKLFPVGDKEEINRVYDFIRNGQYAQYQACNLLMGQLMSEYYKYNRDIKNKEFKARQKEIMTNSNIILKDIDFATGVDTPSAVTQKVKQDFSTALKNGLAKGERTVTNYKRTNPLITRGRNLTFYHEYETYQNFLDKINDSDLAVYIKWVNKILFKVVFGNPHRSLELRSVVQNILEENYKVQGSSIEIDGKSIILNLSISIPKQLRELDENTVVGVDLGIAVPAMCALNNNLYERLAIGNAEDFLRIRTKMQAQRRRLQKSLRNTSGGHGRAKKLKALERLQKAEAHFVETYCHMISKRVVDFALKHNAKYINIENLTGYDTSDFILRNWSYYKLQDYITYKAAKYGIEVRKINPCYTSQICSVCGNWEFGQRKSQSVFECANENCDSYKKYEKIGFNADFNAARNIAMSTLWMETGQVTEKSKQEAREYYGISKKYEQNKNDSENNKVA

SEQ ID NO: 599 - protein, 401 aa, unidentified
  source 1..401: note = Uncultivated organism; MG119-261 effector
MYLVRKIRLIVIGENKEDINEKYKFIRDAQYAQYRGLNYMMGQVGALYYSCDRNISSDEFKTQYMEIFRASNQVVDGIEWGTGIDTRSLIGMRIKQDFSTALKRGLAKGEISLPNYKRSYPLLTKGRDIRFYTDYTEGELVHDMVLEPDFNLYIKWVHKIHFKVVLGNPYRSRFLREELWKVLCGEYKIAGSSLGFDKNGKLILNLSMDIPVNKEKKENMNNVLGVRLAYAKPVVAVVNSTGRCFMIGDGERLIGYRKKMRNNIRRVQQDVTLAAGGSGRAKKLAALNRFKDREANFAKTYNHQISRRIVEIAKNNGCSTIVLEDIGNIYDVDNIVLSNWGYYQLHQQIEYKAQMQDINVVKSQLVAEKQMQTMLETIELDKRDEFIAKSLSQIDIKQKKK

SEQ ID NO: 600 - protein, 479 aa, unidentified
  source 1..479: note = Uncultivated organism; MG119-262 effector
MYITRKIELWVKEDDKNLRNEIWRTIRDYEHMVFKSANLIVTNQLFNQTFTDRIVLTDEELSVKREKIERDIDKLNEKLKDEKDDTKKDKLKENRNKLYRQINQLSTEARKMAEEFYTTSEKNTTYQLIRKEFPTLPSHISASLNDMVTKNYSNEIFDVRLGKRSLRTYRKGMPIPFMKLSLKLKKVDDEICLKWVNNIEFTLHFGRDKSNNQIIVERILKGDYKVGDSSIQLKKGKIFILLVVDIPKEENQLNDEVSVGVDLGLSIPAVCSLNVGKERLFIGSYNDFIRVRTQLQSRKRRLQRSLTLTKGGKGRGKKLKAFDRLKTKERNFVKTYNHTLTNRIVKFAKDNLAATIKLEFLEGYGEDETNSFVLRNWSYYELQTQLQYKAEREGIETVFIDPYHTSQICSFCNHYEQGQRLKQSEFLCKNVDCSNKDKKGENKIINADWNASVNIAKSVKFVTKKSDCEYFKRKKDEKT

SEQ ID NO: 601 - protein, 486 aa, unidentified
  source 1..486: note = Uncultivated organism; MG119-263 effector
MPTEYTCITRKIEVHLHKNGDDAESIALLKEQYAIWNAINDNLYKAANRIVSHCFFNDAYEFRLRLHSPEFDKIAKALKDAEKNKLSKKEIDALKKERKKLFDGFKKQRKLFLSGGVDDGKNKEQNSTYKVIREEFSDVIPSKVANCLNQKVVATYKAFTKDVERGTRTIPNYKKGLPVPFQMTLDKELQLKQRSDGSYYVKFPKQLEWDLYFGRDRSNNLEIVKRILSGEYLVGDSSIQQTKRGKIFLLLVVKIPKKEIALDKNIVVGVDLGINVPLYAALNNNVYGGMGVGSRDQFLKVRMQMYARKREMQRCLRYGTNGGHGRKQKLQALERLEGKERNWVHLQNHIFSKALIDYALKNNAGVIQMEKLSGFGKDKDGEVDDGYKFILRNWSYFELQNMIEYKANAVGIEVRYVDPYHTSQTCSFCGHYEPGQRISQSQFECRNPDCQKGKGKKRSNGEYEGINADWNAARNIALSDKIVSEK

SEQ ID NO: 602 - protein, 440 aa, unidentified
  source 1..440: note = Uncultivated organism; MG119-264 effector
MNITKKIKIEMCAPQDFESLDKEFNYRCQKSQSISLGKTRRDVENAFVEMNSVLWRVYNKVSFKCGTYIYLDKDWGEVSGMNNELYVFTNEVMNEVKKECPSINSVPSSIILRLINDVASKIGSGESKKEIKRGDRKTPEFNKGVPMGIGNKQLRFYKKHNGNEYYVRIYGIWFKTILSKDHQGNRMIINHIMNGDEGYKASDSTLSVKEKGKNIDFILNLSVKIPESKELYDRGIICGIDLGMKNLAVCTICDKSTGDVITNEYTKKELIEKIDDVSIIEVRNKYKAERERIRKKCELTGISGHGRKAKMNRYYDFGHKEKNVARNIYHNVSRKIIKFCKGNNVGVIHMENLKGYGKKECMAASLWGYYELQQMIEYKAKKDGIDVVYVNPKNTSKTCSECGHVNENLALKDRDWICPHCGKHHDRDINAAINIARMLK

SEQ ID NO: 603 - protein, 464 aa, unidentified; identical to SEQ ID NO: 476
  source 1..464: note = Uncultivated organism; MG119-265 effector

SEQ ID NO: 604 - protein, 464 aa, unidentified
  source 1..464: note = Uncultivated organism; MG119-266 effector
MAVKAAKIELEVCDVNKRNSFIESVGKHINNTFKSQYILTLKENKKDIYDDLLKLQKSNFRLHNFIVNKYQNFINSDDKKFKLENNEKNEINHLATKYSKKLCEEYQLNNINSTIPNGILNLIDKNVKKTIKDIKNGKRIWQSYREQVPIPLSQKDAIVKINGKHFTKIDEMNKNYYIVFWGILFKTNLGKDKSNNASIIEKTISGEYKFCDSNVVVEKNIMFLLLSYDAPQKQIELDDDKVLGIDLGIKIPAVGCIYSKTLGKPITYQHNGKDVIESFDETYVKSKYVNKYEKKEIKPSILTIRNAIKIEHEKRKKDAKYICTNGHGRNRKMKFADKISNKEKNTANLYYHNLSRDIINFALKNKCKQINIENLKGFKGDKVLINGWGYFTLQNQIEYKANEYGIKVKKINPKNTSRTCSSCGHVSEKNRIDQEHFKCEHCGLEMNADYNASINIAKSENFVK

SEQ ID NO: 605 - protein, 488 aa, unidentified
  source 1..488: note = Uncultivated organism; MG119-267 effector
MATEYTCITRKIEVHLHKHGDSEEAAQRFKDEYRIWDDINNNLYKAANRIISHCFFNDTYEYRLKLHSPRLQEIEKLLSNPKRNKLSDEDVKQLKAERKHLFADFKKQRQVFLRGGVEDGANPEQNSTYKVVSNEFIDFIPSEVLTNLNQNISSTYREYSLDVERGVRTIPNYKKGIPVPFSIKQKGEIVLKKREDGSIYVRFPKGLEWDLSFGRDRSNNREIVERVLSGQYDVGNSSIQETKNKKRFLLLVVKVPKQVASFDPNRIVGVDLGINVPLYAALNDNEYGGMSIGSRDQFLKVRMRMAAQKRELQHNLRHTTNGGHGRPQKLQALNRLEGKERNWVHLQNHIFSKSIIEYAVRNNAGVIQMERLTGFGRDKNDEVGEDFKFLLRYWSFFELQSMIEYKAKAVGVEVRYINPYHTSQTCSFCGHYEKGQRINQATFICKNPECTKGKGKQRTDGTFEGINADWNAARNIAFSTEFVDKKKK

SEQ ID NO: 606 - protein, 488 aa, unidentified
  source 1..488: note = Uncultivated organism; MG119-268 effector
MANEFTCITRKIEVHLHKHGDSDEAIQRYKEEYRMWDDINNNLYKAANRIVSHCFFNDAYEYRLKLHSPRFQKIEKLLSNPKRNKLSNDDIKELKAERKLLFSDFKSQRQTFLRGGIETGTNQEQNSTYIVIKNEFIACIPSDVLTNLNQNISSTYRKYTLDVERGIRTIPNFKKGIPVPFSIKPNGEIALKKRDDGTIYVRFPKGLEWDLNFGRDRSNNREIVERVLSGQYGVGNSSIQESKNKKQFLLLVVKIPKENRVLDKERIVGVDLGVNTPLYAALNDNEYGGMGIGSREQFLKVRERMNAQKRELQRSLRHSTNGGHGRSQKLQALDRLEGKERNWVHLQNHIFSKSIIEYALKNDAGVIQMERLTGFGRDNNEEVQNEYKYILRYWSYFELQTMIEYKAKAAGIEVRYINPYHTSQTCSFCGHYEKGQRINQSTFICKNPDCTKGKGKQKSDGAYEGINADWNAARNIARSNEFVGKKKK

SEQ ID NO: 607 - protein, 464 aa, unidentified
  source 1..464: note = Uncultivated organism; MG119-269 effector
MAVKAAKIELEVCDVNKRNSFIESVGKHINNTFKSQYILTLKENKKDIYDDLLKLQKSNFRLHNFIVNKYQNFINSDDKKFKLENNEKNEINHLATKYSKKLCEEYQLNNINSTIPNGILNLIDKNVKKTIKDIKNGKRIWQSYREQVPIPLSQKDAIVKINGKHFTKIDEMNKNYYIVFWGILFKTNLGKDKSNNASIIEKTISGEYKFCDSNVVVEKNKMFLLLSYDAPQKQIELDDDKVLGIDLGIKIPAVGCIYSKTLGKPITYQHNGKDVIESFDETYVKSKYVNKYEKKEIKPSILTIRNAIKIEHEKRKKDAKYICTNGHGRNRKMKFADKISNKEKNTANLYYHNLSRDIINFALKNKCKQINIENLKGFKGDKVLINGWGYFTLQNQIEYKANEYGIKVKKINPKNTSRTCSSCGHVSEKNRIDQEHFKCEHCGLEMNADYNASINIAKSENFVK

SEQ ID NO: 608 - protein, 481 aa, unidentified
  source 1..481: note = Uncultivated organism; MG119-270 effector
MATEYTCITRKIEVHLHRHGDSEEAAQRLKEEFRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSDVLTCLNQNISSTYREYALDVESGRRTIPNFKKGIPVPFAIKVHRELALRKREDGSIYIRFPKGLEWDLNFGRDRSNNREIVERVLSGQYDVGNSSIQETKNRKRFLLLVVKIPKESRALIPDRVVGVDLGVNIPLYAALNDNTYGGLSIGSRDQFLKVRMHMAAQKRELQRNLRVATNGGHGRKQKLQALDRLEGKERNWVHLQNHIFSKSIIEYALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 609 - protein, 481 aa, unidentified
  source 1..481: note = Uncultivated organism; MG119-271 effector
MATEYTCITRKIEVHLHRHGDSEEAKQRLAEEYQMWDEINNNLYKAANRIISHCFFNDAYEYRLKLHSPRFKEIEDLLKNAKRKKLTKEDIDELKAERKRLFAEFKKQRQEFLGGSEQNSTYKVATQEFLDKIPSNVLTCLNQNIAGVYREFSLEVERGQRTIPNYKKGIPVPFPLKVHGELMLMRRSDNSVYVRFPKGLEWDLSFGRDRSNNREIVERVLDGRYDVGNSSIQESKNGKRFLLLVVKIPKENRALDANRVVGVDLGVAVPLSAALNDNEYGGMSIGSYDQFTKMRMRMSAQKRELQRNLRHTTNGGHGRAQKLQALERLEGKERNWVHLQNHIFSKSVIEFAIQNGAGAIQMERLAGFGRDKNNEVDEGYKFVLRYWSFFELQSMIEYKAKAAGIEVRYVDPYHTSQTCSFCGHYEKGQRISQSKFVCKCPECTKGKGKQRADGTFEGINADWNAARNIALSTKIVDRKKK

SEQ ID NO: 610 - protein, 481 aa, unidentified
  source 1..481: note = Uncultivated organism; MG119-272 effector
MATEYTCITRKIEVHLHRHGDSEEAAQRLKEEFRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSDVLTCLNQNISSTYREYALDVESGRRTIPNFKKGIPVPFAIKVHRELALRKREDGSIYIRFPKGLEWDLNFGRDRSNNREIVERVLSGQYDVGNSSIQETKNRKRFLLLVVKIPKESRALNPDRVVGVDLGVNIPLYAALNDNTYGGLSIGSRDQFLKVRMHMAAQKRELQRNLRVATNGGHGRKQKLQALDRLEGKERNWVHLQNHIFSKSIIEYALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 611 - protein, 481 aa, unidentified
  source 1..481: note = Uncultivated organism; MG119-273 effector
MANEFVCITRKIEVYLHKHGDDDESKLRYDAEREKWRAINDNLYKAANYITTHCFFNDAFEERLRVQSPEYNRIEKLLRRAKKDNLSYAEIRVLKEERKKLQIQFKQQKKEFLQTSEQNSTYRMVSEEFGDIISSEILTCLNQNISSTYSCYRKEIEYGKRTIPNYKRGIPVPFSIKKKQQLNIKRRADGSIFVRLPFGMEWDLFFGRDRSNNREIVDRVLEQKYDVGNSSLQETQKGKTFLLLVVKIPKQIQELNFNRVVGVDLGINVPLYAALNDNEYGGLSIGSREQFLNVRMRLTKQKRELQYALRNSTNGGHGRKQKLQALERVEKKERNWVHLQNHIFSKSIIEYALKHSAGVIQMENLTGFGRDVDNEVEEDYKFILRYWSFYELQQLIEYKANAAGITVRYINPYHTSQRCSFCGHYEQGQRINQSTFICKNNECKKGKGKTKSDGTYLGINADWNAARNIALSVDFVDKRKK

SEQ ID NO: 612 - protein, 489 aa, unidentified
  source 1..489: note = Uncultivated organism; MG119-274 effector
MANTEFTCITRKIEVHLHRHGDSDEAALRLKNEYHFWDEINDNLYKAANRIISHCFFNDTYEYRLKLHSPRFQEIEKLLKNAKRNKLTDEEIKELKAERKLLFSDFKKQRYTFLRGGIEDGANPEQNSTYRVVSNEFIDTIPSEVLTNLNQNISSTYKNYTIDVERGLRTIPNYKRGMPVPFSIKKHGVLALRKREDGSIYVAFPKGLEWDLSFGRDRSNNREIVERVLSGLYDVGNSSIQEAKNGKRFLLLVVKIPKEVKILDTNRVVGVDLGVAVPLYAAISDNEYGGMSIGSYDQFIKVRMRMNAQKREMQHNLRHTTNGGHGRTQKLQALERLEGKERNWVHLQNHIFSKSIIEYALQNNAGAIQMERLTGFGHDKNNEVDEGYKFILRYWSFFELQTMIEYKAKAAGIEVRYIDPYHTSQTCSFCGHYEKGQRLNQSKFVCKNPDCVKGKGKQMSDGSFEGINADWNAARNIALSTKIVDRKKK

SEQ ID NO: 613 - protein, 488 aa, unidentified
  source 1..488: note = Uncultivated organism; MG119-275 effector
MATEYTCITRKIEVHLHRHGEDEEAKQRLIDDYRVWDTINDNLYKAANRIVSHCFFNDAFEYRLKIHSPRFQEIEKLLKYSKRNKLTDEDIKQLKAERKQMFADFKKQRHTFLRGGVAEGANPEQNSTYKVISNEFLEVIPSEILTNLNQNISSTYKNYSLDVERGIRTIPNYKRGIPVPFSIKQRGELMLKRRDDGSIFIRFPMGLEWDLSFGRDRSNNREIVERVLSGQYDVGNSSIQESKNRKRFLLLVVKIPKESRNLNPDRIVGVDLGINIPLYAALNDNDYGGMGIGSREQFLNMRMRMAAKKRELQRNLRQSTNGGHGRKQKLQALERLEGKERNWVHLQNHIFSKSIIEYALKNNAGAIQMERLTGFGRDKNDEVDSNFKFILRYWSFFELQTMIEYKANAAGIEVRYVDPYHTSQTCSFCGHYEKGQRLNQSTFVCKNPDCEKGKGKKLSDGTYQGINADWNAARNIALSDKIVDRKKK

SEQ ID NO: 614 - protein, 488 aa, unidentified
  source 1..488: note = Uncultivated organism; MG119-276 effector
MATEYTCITRKIEVHLHKHGEGEEATKRLQEEYRIWDDINNNLYKAANRIVSHCFFNDTYEYRLKLHSPRFQEIEKKLSNPKRNKLSDEEVKQLKAERKLLFADFKKQRQVFLRGGIEEGANPEQNSTYKVVSKEFIDTIPSEVLTNLNQNISSTYREYALDVERGVRTIPNYKKGIPVPFSIKQKGDIVLKKRDDGTIYVRFPKGLEWDLSFGRDRSNNREIVERVLSGEYDVGNSTIQETKNRKRFLLLVVKIPKQTVVFNPNRIVGVDLGINVPLYAALNDNEYGGMGIGSRDQFLKVRMRMVAQKRELQRNLRHTTNGGHGRTQKLQALDRLEGKERNWVHLQNHIFSKSIIEYAVKNNAGIIQMERLTGFGRDDKEEVQDEYKFILRYWSFFELQTMIEYKAKAAGIEVRYINPYHTSQTCSFCGHYEKGQRINQGTFICKNPECVKGKGKQKSDGSYEGINADWNAARNIAKSTEVVDKKRK

SEQ ID NO: 615 - protein, 486 aa, unidentified
  source 1..486: note = Uncultivated organism; MG119-277 effector
MAAEFTCITRKIEVHLHRHGEGEEAEMRLKEEYRIWDEINDNLYKAANRVVSHCFFNDSYEYRLKLHSPRFQEIEKLLKNAKRNKLSNDDIRELKEERSLLFSDFKKQRLAFLQGNSGKGSEQNSTYKVISNEFVDVIPSEVLTNLNQDISSTYKNYALEVERGARTIPNYKKGIPVPFSIKQSGELRLRKREDGSVYITFPRGLEWDLNFGRDRSNNREIVERVLSGLYDVGNSSIQETKSRKRFLLLVVKIPKEASKLDKNKVVGVDLGVAVPLYAALNDNQYGGMSIGSYEQFTKVRQRMFAQKKELQRNLRHTTNGGHGRKQKLQALERMEGKERNWVHLQNHIFSKSLIEYALGNKAGVVQMERLTGLGRDVNDELGEGYKFVLRYWSFFELQTMIEYKAKAAGIEVRYINPYHTSQTCSFCGHYEKGQRINQSTFICKNPDCIKGKGERKSDGSYKGINADWNAARNIALSKEYVEKKKK

SEQ ID NO: 616 - protein, 466 aa, unidentified
  source 1..466: note = Uncultivated organism; MG119-278 effector
MDWDKLSLHSKEEYMILTRKLALKARKKEDLGYLYTLNNSVFKAYNTVVNTLYFSYLFKDKITLTDAELAKRREKISRELVKIRGKISLIDKDDKTKLDNLKKRRKGFYNKLNTLTREARDRAEEYYTTSEKNYGYQIISKEFPDIPSYIRACINVEVYANFTKDINRIRNGEISVRTYRKGTPIPFMASAGKFIKDEDNIIFKWLKGIEFDIQFGRDKSARKSEVEKIISGDLKMSDSKIQIKNRKIFLLLAIDQPVEKKDLNMNITVGVDVGIKYPAYVSTNSGIQHKHIGSIDDLFKLRMQIQKRRQRLQKQIKSAKGGKGRGKKLKALNRMREKERNYVRTYNHYISREVVNFALENRAGTIIMEDLSGFGVNYKKKNTILRNWSYFELQSMIDYKAKLMGIKVEKIKPEYTSQTCALCGMKGKREEREIFICLNSECKNFEKPINADYNAAMNISRRGIKK

SEQ ID NO: 617 - protein, 453 aa, unidentified
  source 1..453: note = Uncultivated organism; MG119-279 effector
MILTRKLKLELIDRDNLEYLYNLNYLTFKAYNTAVNALYFSSLFKDIIFDSDKEIVEKRKEIIQQLKSIDEEIFQAKDEDKENKDELKRQKTYLQSMYKSLFRDVQKTAEEFYGGSERNLAYKIINKDYSELPSYIKAAIGADVYANYKSDIGQIRRGEISLRTYRKGIPIPFMKSAGKFIKEDDNIFFQWVKGIKFKIIFGRDKSARQVEVERILACKLKISDSKIQFKNKKIFLLLAINQSIDKKELDKNIAVGVDLGIKYPAYVSTNSGIQHKHIGSYDDLFKLRLQIQERKKRLQKNLKYASGGKGRKKKLKALNRIETKERNYIRTYNHSISKEIVNFTLQNKAANIIIEDLSGFGSRDDNKKRVLRNWSYFELQSMIKYKAELKGIKVIEISPKYTSQTCAICGERGDRDTQNSFVCSNPECENFNQSVNADYNASFYIAKTGLEKK

SEQ ID NO: 618 - protein, 453 aa, unidentified
  source 1..453: note = Uncultivated organism; MG119-280 effector
MILTRKLKLELIDRDNLEYLYNLNYLTFKAYNTAVNALYFSSLFKDIIFDSDKEIVEKRKEIIQQLKSIDEEIFQAKDEDKENKDELKRQKTYLQSMYKSLFRDVQKTAEEFYGGSERNLAYKIINKDYSELPSYIKAAIGADVYANYKSVIGQIRRGEISLRTYRKGIPIPFMKSAGKFIKEDDNIFFQWVKGIKFKIIFGRDKSARQVEVERILACKLKISDSKIQFKNKKIFLLLAINQSIDKKELDKNIAVGVDLGIKYPAYVSTNSGIQHKHIGSYDDLFKLRLQIQERKKRLQKNLKYASGGKGRKKKLKALNRIETKERNYIRTYNHSISKEIVNFTLQNKAANIIIEDLSGFGSRDDNKKRVLRNWSYFELQSMIKYKAELKGIKVIEISPKYTSQTCAICGERGDRDTQNSFVCSNPECENFNQSVNADYNASFYIAKTGLEKK

SEQ ID NO: 619 - protein, 453 aa, unidentified
  source 1..453: note = Uncultivated organism; MG119-281 effector
MILTRKLQLKIIDVKNWLFLNELNNNVFKAYNDVVNTHYFSTLFKDKIALTDEEINSKTIKINKKLNKNKEIIKEAKDDKKKIETLKKEQQKLYNTLRILNREARDKAEVFYTTGDKNFGYQMLSRKFPEIPSTIRTCINADVYANFTKEINDIRSGKRAVRTYRKGVPIPFQLDKSKEIFVQKNDKIIFRWIKDIDFEIIFGRDKSARQSEVEKIISGDYKMSDSKLQIKNKKIFLLLALNQPEEKLQLDKSVTIGVDLGIVFPAYVSTNSGIQHQRIGSSDDLFKLRMQIQKRRRRLQKDIKSAHGGKGRKKKLKALERLRQKERNYVRTYNHFISREIINFAIQQKAATIFIEDLSGFGKNKEKGNIILRNWSYFELQSMIEYKAKLKGVEVKKVDPKYTSQICSICGEKGKRESQKDFLCLNKKCENFGKKILADYNAAWNIAKGGIKI

SEQ ID NO: 620 - protein, 458 aa, unidentified
  source 1..458: note = Uncultivated organism; MG119-282 effector
MNIARKIKMEIISPSRSEEILKKYNINRKEKMMISSGQSIREIEQSLLDMNEILPRACNMTMVDFHKIIHSNLSDDEKCTLIKNRGIIGKKPGEKVNNDPNDIRVHLKDNLNLLASEYGSINNVPSSIYVRGYTDMTNGIIEKSGKIKREILRYDRVLPEFKKGMPMGLSSQRIRIYPNEDGEFYFVIWGVLFKTILRKDKQKNRDILYEIINNNDYKLSDSTLSVFKNGKNTEIYFNMTVKIPESKELYDRGIICGLDLGMKTLVTCAVCDKATGNVITDEHSKKEIIGKIGDGSTIIESRNKYKIEGERIRKKCEMMGVNSHGRNAKMEEYYKYIHNKERNAAKNIYHTVSRKVIKLCKEYNAGVIQMENLEGYGKKECVAASLWGYYELQKMIEYKANKEGIEVKYVNPKNTSKTCSECHGVNESLTLKDREWVCPHCGKHHDRDVNAAINIARM

SEQ ID NO: 621 - protein, 458 aa, unidentified
  source 1..458: note = Uncultivated organism; MG119-283 effector
MNIVRKIKMEIISPSRSEEILKKYNINRKEKMMISTGQSIREIEQSLLDMNEILPRACNMTMVDFHKIIHSNLSDDEKCALIKNRGIIGKKPGEKVNNDPNDIRVHLKDNLNLLASEYGSINNVPSSIYVRGYTDMTNGIIEKSGKIKREILRYDRVLPEFKKGMPMGLSSQQIRIYPNEDGEFYFVIWGVLFKTILRKDKQKNRDILYGIINNNDYKLSDSTLSVFKNGKNTEIYFNMTVKIPESKELYDRGIICGLDLGMKTLVTYAICDKATGNVITDEHSKKEIIGKIGDGSTIIEFRNKYKIEGERIRKKCEMMGVNSHGRNAKMEEYYKYIHNKERNAAKNIYHTVSRKVIKLCKENNAGVIQMENLEGYGKKECIAASLWGYYELQKMIEYKAKKEGIEVKYVNPKNTSKTCSECHGVNESLTLKDREWVCPHCGKHHDRDVNAAINIARM

SEQ ID NO: 622 - protein, 458 aa, unidentified
  source 1..458: note = Uncultivated organism; MG119-284 effector
MNIVRKIKMEIISPSRSEEILKKYNINRKEKMMISTGQSIREIEQSLLDMNEILPRACNMTMVDFHKIIHSNLSDDEKCALIKNRGIIGKKPGEKVNNDPNDIRVHLKDNLNLLASEYGSINNVPSSIYVRGYTDMTNGIIEKSGKIKREILRYDRVLPEFKKGMPMGLSSQQIRIYPNEDGEFYFVIWGVLFKTILRKDKQKNRDILYEIINNNDYKLSDSTLSVFKNGKNTEIYFNMTVKIPESKELYDRGIICGLDLGMKTLVTCAVCDKATGNVITDEHSKKEIIGKIGDGSTIIESRNKYKIEGERIRKKCEMMGVNSHGRNAKMEEYYKYIHNKERNAAKNIYHTVSRKVIKLCKENNAGVIQMENLEGYGKKECVAASLWGYYELQKMIEYKAKKEGIEVKYVNPKNTSKTCSECHGVNESLTLKDREWVCPHCGKHHDRDVNAAINIARM

SEQ ID NO: 623 - protein, 486 aa, unidentified
  source 1..486: note = Uncultivated organism; MG119-285 effector
MADKQKEVIITRKIEVHLHHDPVADPDKVEYNRHWEVWHTINNNLYKAANRIVSHKFFNDEYEHRLQLHSPEFQEINRKLARAKKEQLSAEEIAALRSRIKELYAQFANEKRTFLRGGVAVGPNPEQNSTDKVVRDEFSDVVPTEILTCLNQSVSSTYSEYKKQIARGERAIPNYKKGMPVPIRMKANGQLKLRRRPDNSIYVLTPRGLEWDLSFGRDRSNNREIVERVLSGQYDVGGSSIQEKGRKIFLLLVVKLPASQNELNPERVVGVDLGLNIPLYAALNDNEYGGLSIGSRDKFMKERERMSARRRELQRSLRISTQGGHGRSHKLKALDQFEEKERNWVHTQNHIFSRQVVEYAKSNGAGVIQMESLKGSGKDKEEHIEQGFKYVVRYWSYFELQTLIQQKAEREGIEVRKVNPYHTSQTCSFCGHYEKGQRVSQSTFICKNPACAHGKGKQLKDGTFAGINADWNAARNIALSTEWVKE

SEQ ID NO: 624 - protein, 481 aa, unidentified
  source 1..481: note = Uncultivated organism; MG119-286 effector
MATEYTCITRKIEVHLHRHGDSEEALQRYKEEFRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSHVLTCLNQNISSTYREYALDVEHGRRTIPNFKKGIPVPFPIKATGELLLRKREDGSIYIRFPKGLEWDLNFGRDRSNNREIVERVLSGQYDVGNSSIQETKNRKRFLLLVVKIPKESRTLNPDRVVGVDLGVNIPLYAALNDNTYGGLSIGSRDQFLKVRMRMAAQKRELHRNLRVATNGGHGRKQKLQALDRLEGKERNWVHLQNHIFSKSIIEYALRNEAGAIQMERLTGFGHDRNDEVDEGFKFILRYWSFFELQSMIEYKAKAAGIEVRYVDPYHTSQTCSFCGHYEKGQRVNQATFICKNPDCTKGKGKEHSDGTFEGINADWNAARNIALSDKIVERKKK

SEQ ID NO: 625 - RNA, 172 nt, synthetic construct
  source 1..172: mol_type = other RNA; note = 119-28 sgRNA1_Mouse_Alb
  modified_base 1: mod_base = um
  modified_base 2: mod_base = um
  modified_base 3: mod_base = gm
  modified_base 1^2: mod_base = OTHER; note = Phosphorothioate bond
  modified_base 2^3: mod_base = OTHER; note = Phosphorothioate bond
  modified_base 3^4: mod_base = OTHER; note = Phosphorothioate bond
  modified_base 170: mod_base = cm
  modified_base 171: mod_base = um
  modified_base 172: mod_base = OTHER; note = 2'-O-Methyladenosine
  modified_base 169^170: mod_base = OTHER; note = Phosphorothioate bond
  modified_base 170^171: mod_base = OTHER; note = Phosphorothioate bond
  modified_base 171^172: mod_base = OTHER; note = Phosphorothioate bond
ttgaaataaaatgaatttcaaaccccttcgggggagggcgcgttggagcgccttagtttgaggtgcagaatcaaaaaaactgcgacgatggaggtcgtttcagtctctgtacactcaaaaaattcacttgagaaatcaagtgaatatccaacaagattgatgaagacaacta

SEQ ID NO: 626 - RNA, 172 nt, synthetic construct
  source 1..172: mol_type = other RNA; note = 119-28 sgRNA2_Mouse_Alb
  modified_base 1: mod_base = um
  modified_base 2: mod_base = um
  modified_base 3: mod_base = gm
  modified_base 170: mod_base = OTHER; note = 2'-O-Methyladenosine
  modified_base 171: mod_base = cm
  modified_base 172: mod_base = OTHER; note = 2'-O-Methyladenosine
  modified_base 1^2: mod_base = OTHER; note = Phosphorothioate bond
  modified_base 2^3: mod_base = OTHER; note = Phosphorothioate bond
  modified_base 3^4: mod_base = OTHER; note = Phosphorothioate bond
  modified_base 169^170: mod_base = OTHER; note = Phosphorothioate bond
  modified_base 170^171: mod_base = OTHER; note = Phosphorothioate bond
  modified_base 171^172: mod_base = OTHER; note = Phosphorothioate bond
ttgaaataaaatgaatttcaaaccccttcgggggagggcgcgttggagcgccttagtttgaggtgcagaatcaaaaaaactgcgacgatggaggtcgtttcagtctctgtacactcaaaaaattcacttgagaaatcaagtgaatatccaacggtcagtgaagagaagaaca

SEQ ID NO: 627 - RNA, 172 nt, synthetic construct
  source 1..172: mol_type = other RNA; note = 119-28 sgRNA3_Mouse_Alb
  modified_base 1: mod_base = um
  modified_base 2: mod_base = um
  modified_base 3: mod_base = um
  modified_base 170: mod_base = cm
  modified_base 171: mod_base = cm
  modified_base 172: mod_base = OTHER; note = 2'-O-Methyladenosine
  modified_base 1^2: mod_base = OTHER; note = Phosphorothioate bond
  modified_base 2^3: mod_base = OTHER; note = Phosphorothioate bond
  modified_base 3^4: mod_base = OTHER; note = Phosphorothioate bond
  modified_base 169^170: mod_base = OTHER; note = Phosphorothioate bond
  modified_base 170^171: mod_base = OTHER; note = Phosphorothioate bond
  modified_base 171^172: mod_base = OTHER; note = Phosphorothioate bond
ttgaaataaaatgaatttcaaaccccttcgggggagggcgcgttggagcgccttagtttgaggtgcagaatcaaaaaaactgcgacgatggaggtcgtttcagtctctgtacactcaaaaaattcacttgagaaatcaagtgaatatccaacagtgtagcagagaggaacca

SEQ ID NO: 628 - RNA, 172 nt, synthetic construct
  source 1..172: mol_type = other RNA; note = 119-28 sgRNA4_Mouse_Alb
  modified_base 1: mod_base = um
  modified_base 2: mod_base = um
  modified_base 3: mod_base = gm
  modified_base 170: mod_base = OTHER; note = 2'-O-Methyladenosine
  modified_base 171: mod_base = gm
  modified_base 172: mod_base = OTHER; note = 2'-O-Methyladenosine
  modified_base 1^2: mod_base = OTHER; note = Phosphorothioate bond
  modified_base 2^3: mod_base = OTHER; note = Phosphorothioate bond
  modified_base 3^4: mod_base = OTHER; note = Phosphorothioate bond
  modified_base 169^170: mod_base = OTHER; note = Phosphorothioate bond
  modified_base 170^171: mod_base = OTHER; note = Phosphorothioate bond
  modified_base 171^172: mod_base = OTHER; note = Phosphorothioate bond
ttgaaataaaatgaatttcaaaccccttcgggggagggcgcgttggagcgccttagtttgaggtgcagaatcaaaaaaactgcgacgatggaggtcgtttcagtctctgtacactcaaaaaattcacttgagaaatcaagtgaatatccaactctgtggaaacagggagaga